(19) United States
(12) Patent Application Publication
(10) Pub. No.: US 2020/0268897 A1
(43) Pub. Date: Aug. 27, 2020

(54) Phosphatase Binding Compounds and Methods of Using Same

(71) Applicants: Yale University, New Haven, CT (US); Halda Therapeutics OpCo, Inc., Branford, CT (US)

(72) Inventors: Craig Crews, New Haven, CT (US); Samuel W. Gerritz, Guilford, CT (US); Kyle J. Eastman, Killingworth, CT (US); Katherine J. Kayser-Bricker, Branford, CT (US); Jinshan M. Chen, Madison, CT (US); David E. Puleo, Norwalk, CT (US)

(21) Appl. No.: 16/737,148

(22) Filed: Jan. 8, 2020

Related U.S. Application Data
(60) Provisional application No. 62/789,885, filed on Jan. 8, 2019.
(60) Provisional application No. 62/925,064, filed on Oct. 23, 2019.

(51) Int. Cl. A61K 47/64; A61K 47/60; A61K 47/54

(52) U.S. Cl. CPC A61K 47/64; A61K 47/545; A61K 47/60

(57) ABSTRACT

The present invention provides bifunctional compounds that efficiently dephosphorylate certain phospho-activated target proteins. Such target proteins can be any protein involved in the pathway of a disease or disorder, such as but not limited to cancer, neurodegeneration, metabolic disease, diabetes, insulin resistance, and so forth.

### CROSS-REFERENCE TO RELATED APPLICATIONS

The present application claims priority under 35 U.S.C. § 119(e) to U.S. Provisional Patent Applications No. 62/925,064 filed Oct. 23, 2019, and No. 62/789,885 filed Jan. 8, 2019, all of which are hereby incorporated by reference in their entireties herein.

### STATEMENT REGARDING FEDERALLY SPONSORED RESEARCH OR DEVELOPMENT

This invention was made with government support under CA197589 awarded by National Institutes of Health. The government has certain rights in the invention.

### SEQUENCE LISTING

The instant application contains a Sequence Listing which has been submitted in ASCII format via EFS-Web and is hereby incorporated by reference in its entirety. Said ASCII copy, created on Jan. 8, 2020, is named 047162-7197US1_SequenceListing.txt and is 2.53 kilobytes in size.

### BACKGROUND OF THE INVENTION

Kinase inhibitors have transformed medicine, as those compounds can be used to block or interfere with key disease pathways, especially in the context of abnormal cellular proliferation, such as cancer. Several kinase inhibitors have been approved for treatment of cancer, for example: imatinib (chronic myeloid leukemia, or CML), erlotinib (non-small cell lung cancer, or NSCLC), lapatinib (breast cancer), and vemurafenib (melanoma).

However, kinase inhibition is not the definitive therapeutic solution to cancer treatment. Cellular proliferation pathways tend to be redundant, and thus inhibition of a particular kinase in a given pathway may not be sufficient to prevent activation of a downstream target in that given pathway. Further, even if inhibition of a kinase blocks signaling in a certain pathway, this blockage may trigger a negative feedback mechanism in the pathway, which acts to restate signaling through the pathway.

There is thus a need in the art to identify compounds that selectively block activation of phospho-activated target proteins. These compounds should also maintain the negative feedback regulation associated with these target proteins, thus avoiding the restatement of pathway signaling. These compounds would be useful to treat and/or prevent diseases or disorders, such as cancer, metabolic disease, and/or neurodegenerative disease. The present invention addresses this need.

### BRIEF SUMMARY OF THE INVENTION

The present invention provides a compound of formula (I), or a salt, solvate, prodrug, isotopically labelled derivative, stereoisomer, tautomer, or geometric isomer thereof, and any mixtures thereof:

(protein phosphatase ligand)-LINKER-(target protein ligand) (I);

wherein the protein phosphatase ligand binds to a protein phosphatase, such that the protein phosphatase ligand does not significantly inhibit the phosphatase activity of the protein phosphatase; wherein the target protein ligand binds to a target protein; wherein the LINKER is selected such that it allows for the compound to bind simultaneously to the protein phosphatase and the target protein; wherein, when the compound is simultaneously bound to the protein phosphatase and the target protein, the protein phosphatase is capable of dephosphorylating the target protein. The present invention further provides certain compounds, or a pharmaceutically acceptable salt thereof, as defined and/or disclosed elsewhere herein.

The present invention further provides pharmaceutical compositions comprising at least one compound as defined and/or disclosed elsewhere herein and at least one pharmaceutically acceptable carrier.

The present invention further provides method of treating or preventing a disease associated with and/or caused by overphosphorylation, undesirable phosphorylation, and/or uncontrolled phosphorylation of a target protein in a subject, the method comprising administering to the subject a therapeutically effective amount of at least one compound as defined and/or disclosed elsewhere herein. The present invention further provides methods of dephosphorylating a target protein having a phosphate group, comprising exposing or contacting the target protein to a compound as defined and/or disclosed elsewhere herein, to thereby dephosphorylate the target protein

### BRIEF DESCRIPTION OF THE DRAWINGS

The following detailed description of specific embodiments of the invention will be better understood when read in conjunction with the appended drawings. For the purpose of illustrating the invention, specific embodiments are shown in the drawings. It should be understood, however, that the invention is not limited to the precise arrangements and instrumentalities of the embodiments shown in the drawings.

FIG. 1 illustrates a non-limiting synthesis of compounds of the invention.

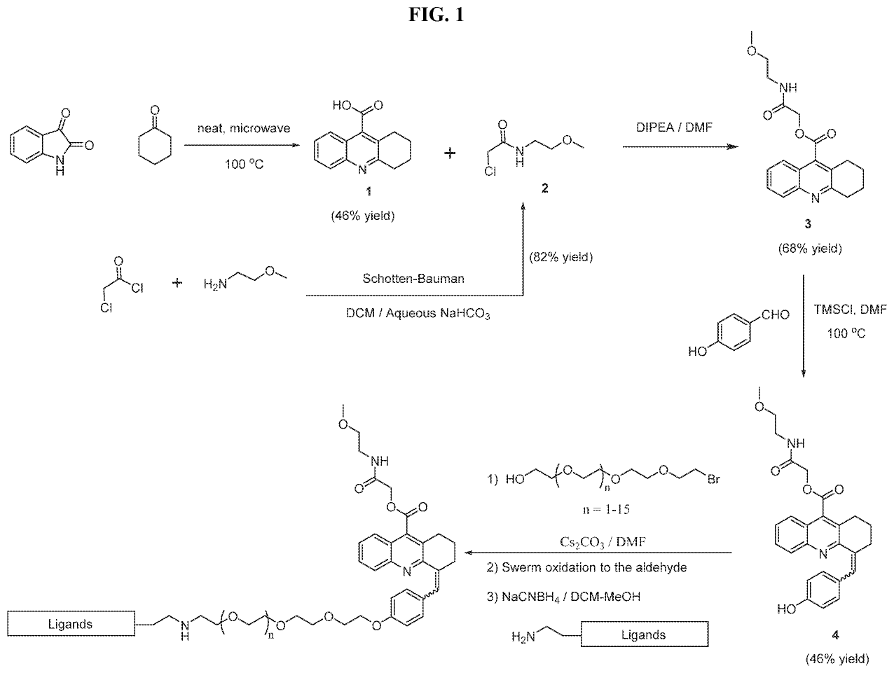

FIG. 2 illustrates a non-limiting synthesis of compounds of the invention.

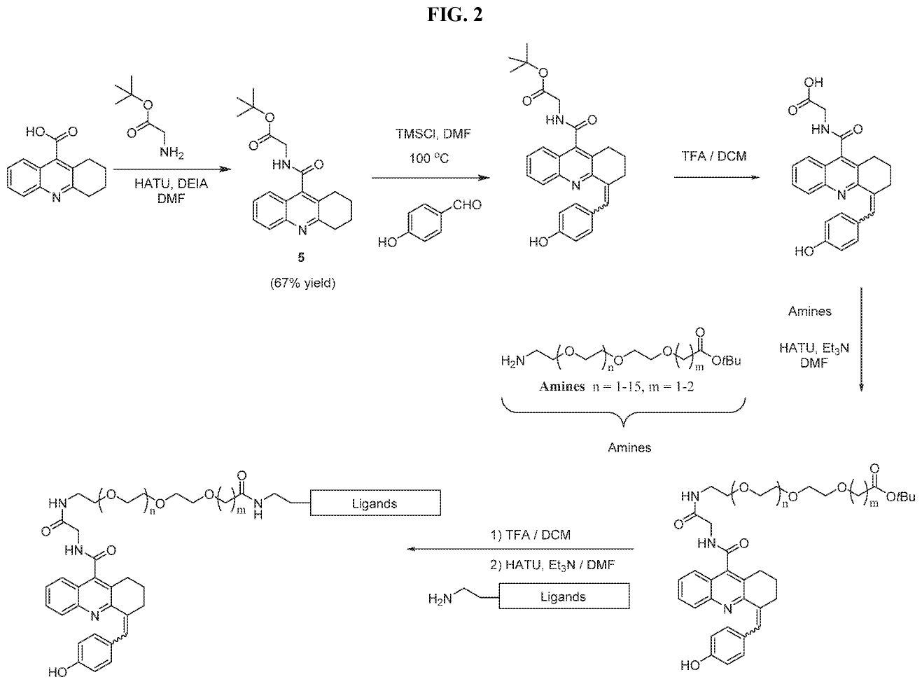

FIG. 3 illustrates a non-limiting synthesis of compounds of the invention.

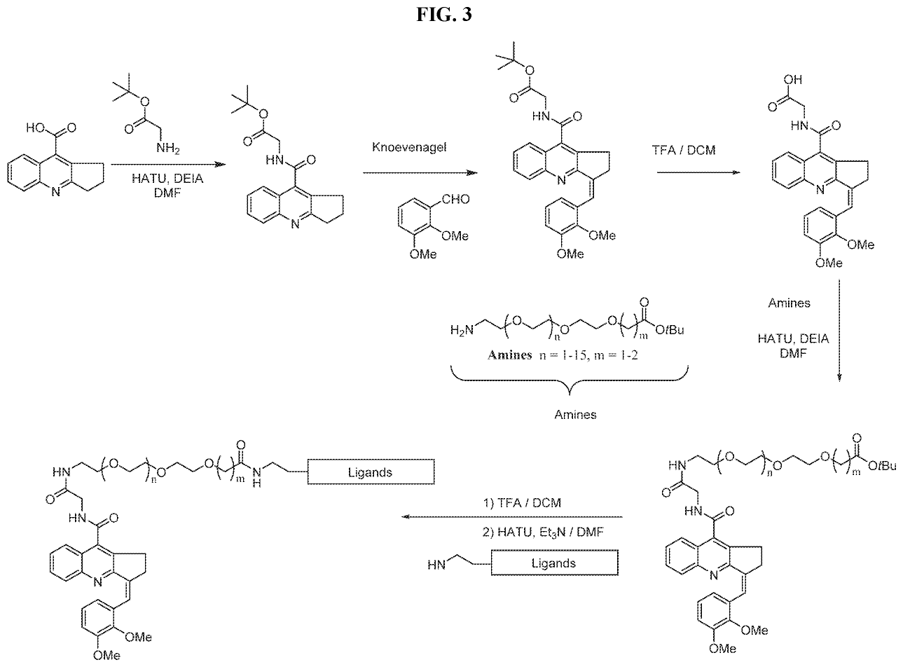

FIG. 4 illustrates a non-limiting synthesis of compounds of the invention.

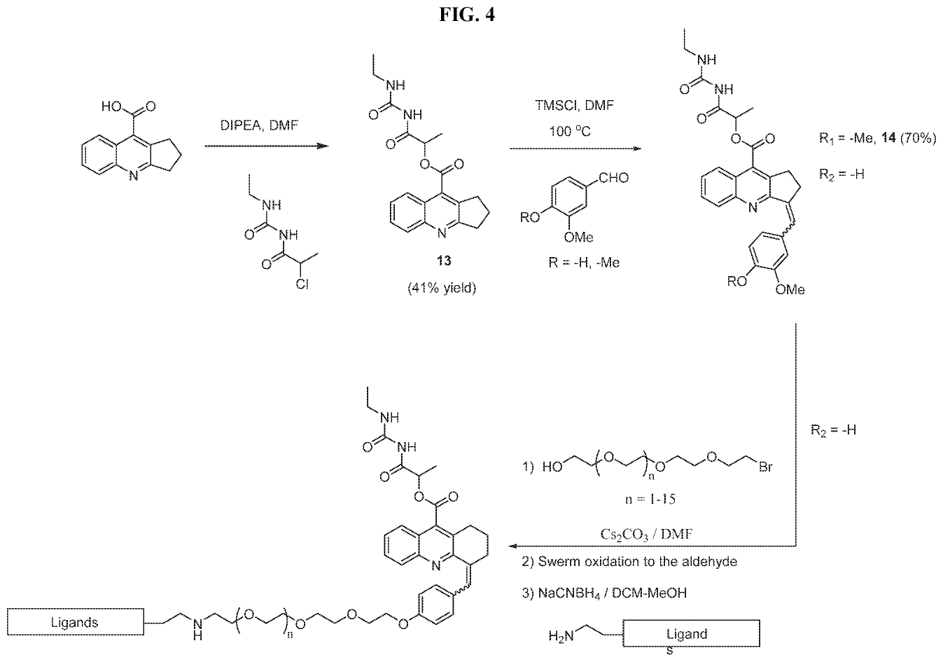

FIG. 5 illustrates a non-limiting synthesis of compounds of the invention.

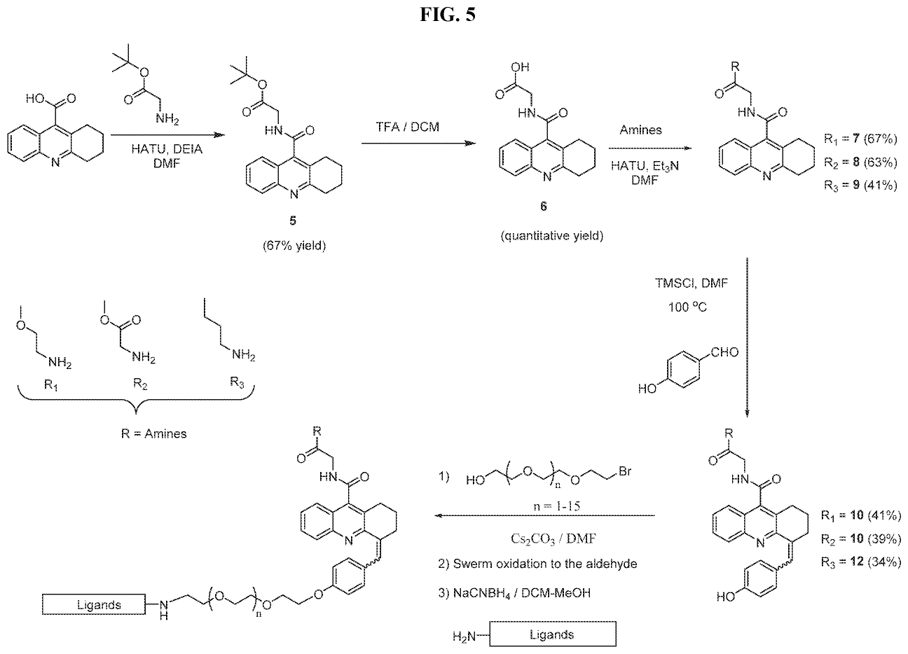

FIG. 6 illustrates a non-limiting synthesis of compounds of the invention.

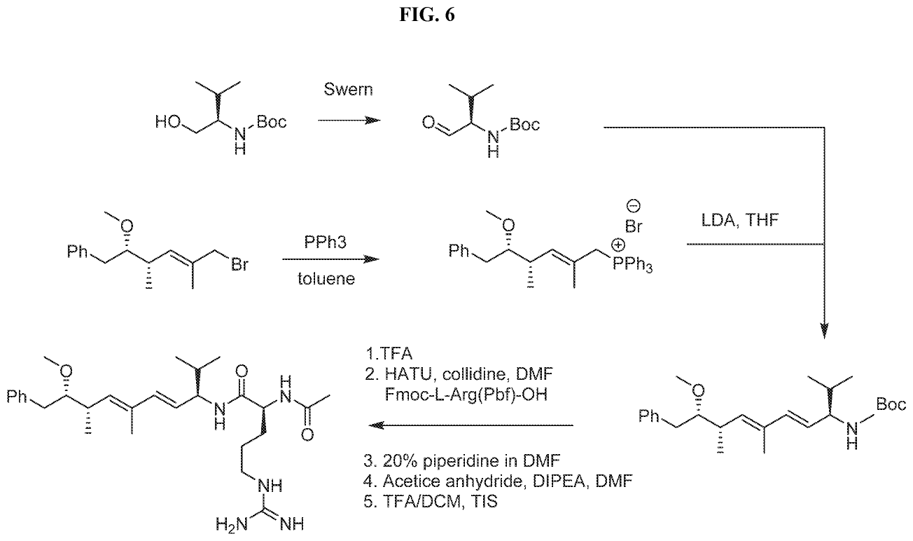

FIG. 7 illustrates a non-limiting LINKER-(protein phosphatase ligand) group contemplated within the invention. The peptide incorporated in the top four molecules is a protein phosphatase 1 (PP1 binder), comprising the peptide of amino acid sequence of SEQ ID NO:2. The peptide incorporated in the bottom molecule is a negative control (comprising the peptide of amino acid sequence of SEQ ID NO:6) and does not bind to PP1. In a non-limiting embodiment, each peptide is amidated at the C-terminus.

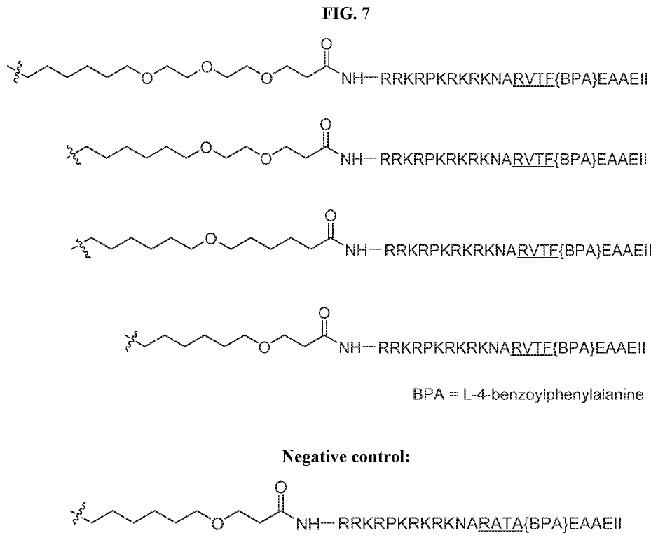

FIG. 8 illustrates a non-limiting LINKER-(protein phosphatase ligand) group contemplated within the invention. The peptide incorporated in the top four molecules is a protein phosphatase 2A (PP2A binder), comprising the peptide of amino acid sequence of SEQ ID NO:5. The peptide incorporated in the bottom molecule is a negative control (comprising the peptide of amino acid sequence of SEQ ID NO:7) and does not bind to PP2A. In a non-limiting embodiment, each peptide is amidated at the C-terminus.

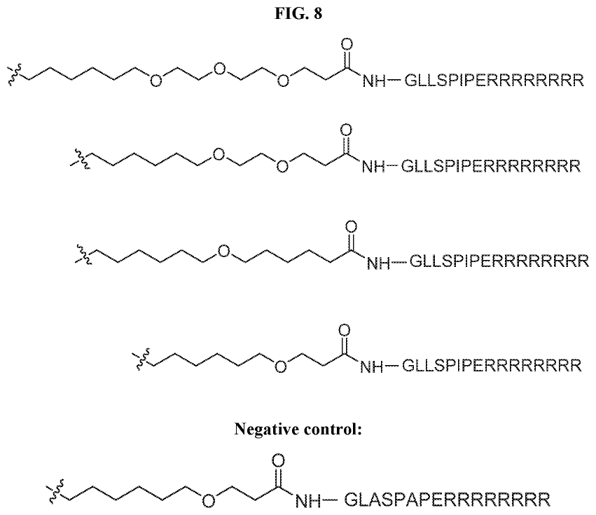

### DETAILED DESCRIPTION OF THE INVENTION

The present invention relates to the discovery of bifunctional compounds that efficiently dephosphorylate certain phospho-activated target proteins. Such target proteins can be any protein involved in the pathway of a disease or disorder, such as but not limited to cancer, neurodegeneration, metabolic disease, diabetes, insulin resistance, and so forth.

In one aspect, the compounds of the invention comprise a ligand that binds to a protein phosphatase (“protein phosphatase ligand”) that is linked through a chemical linker to a ligand that binds to a target protein (“target protein ligand”). This bifunctional compound of the invention can thus simultaneously bind to the target protein (through the target protein ligand) and to a protein phosphatase (through the protein phosphatase ligand). Such simultaneous binding brings the protein phosphatase in close spatial proximity to target protein, allowing for the protein phosphatase to dephosphorylate the target protein. Without wishing to be limited by any theory, this binding-followed-by-dephosphorylation process represents an event-driven pharmacology approach. Instead of counting on high concentrations of drug to drive target saturation and inactivation (as expected in an occupancy-driven pharmacology observed for kinase inhibitors), the present invention provides for transient interaction between the target protein and the phosphatase through simultaneous binding to a compound of the invention, wherein even sub-stoichiometric levels of the compound allow for dephosphorylation of the target protein.

In certain embodiments, the protein phosphatase ligand useful within the invention does not bind to the active site of the protein phosphatase, and thus does not inhibit the phosphatase activity of the enzyme.

In certain embodiments, any known linker is contemplated within the invention, as long as the compound of formula (I) can simultaneously bind to the target protein (through the target protein ligand) and to the protein phosphatase (through the protein phosphatase ligand).

In certain embodiments, the compounds of the invention can be used to treat diseases associated with overphosphorylation, uncontrolled or unregulated phosphorylation, and/or abnormal phosphorylation of a target protein. In other embodiments, the compounds of the invention can be used to treat a cancer that is associated with overphosphorylation, uncontrolled or unregulated phosphorylation, and/or abnormal phosphorylation of a target protein.

The present description provides compounds comprising a ligand, e.g., a small molecule ligand and/or a peptide ligand, which is capable of binding to a protein phosphatase. As described elsewhere herein, the compound of the invention further comprises a target protein ligand, such that the target protein is placed in proximity to the protein phosphatase to effect dephosphorylation of the target protein.

In certain embodiments, “small molecule” means that the molecule is non-peptidyl, i.e., it is not generally considered a peptide, e.g., comprises fewer than 4, 3, or 2 amino acids. Further, in other embodiments, a small molecule has a molecular weight that is lower than about 2,500 Da, 2,000 Da, 1,500 Da, 1,000 Da, 750 Da, or 500 Da.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, specific methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of +20% or +10%, more preferably +5%, even more preferably +1%, and still more preferably +0.10% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, and so forth) from those organisms, tissues, cells or components thereof that display the “normal” (expected) respective characteristic. Characteristics that are normal or expected for one cell or tissue type might be abnormal for a different cell or tissue type.

A disease or disorder is “alleviated” if the severity of a symptom of the disease or disorder, the frequency with which such a symptom is experienced by a patient, or both, is reduced.

As used herein, the term “amino acid” refers to any natural or non-natural compound having a carboxyl group and an amino group in a molecule. An “amino acid” as used herein is meant to include both natural and synthetic amino acids, and both D- and L-amino acids. Amino acids contained within the peptides, and particularly at the carboxy- or amino-terminus, can be modified by methylation, amidation, acetylation or substitution with other chemical groups which can change a peptide's circulating half-life without adversely affecting activity of the peptide. Additionally, a disulfide linkage may be present or absent in the peptides.

In certain embodiments, the peptides of the invention are further modified, by using methods such as but not limited to: methylation of one or more NH groups in the peptide backbone; amidation and/or esterification of the C-terminus carboxyl group and/or any side chain carboxyl group; alkylation, acylation, carbamoylation and/or sulfonylation of the N-terminus amino group and/or any side chain amino group; and any other peptide modification known in the art.

As used herein, the term “specifically bind” or “specifically binds,” as used herein, is meant that a first molecule (e.g., a target protein or a phosphatase) preferentially binds to a second molecule (e.g., a target protein ligand or a phosphatase ligand, respectively), but does not necessarily bind only to that second molecule. In certain embodiments, the binding is reversible. In other embodiments, the binding is irreversible (or non-reversible).

The term “cancer” refers to the physiological condition in a subject typically characterized by unregulated cell growth. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small cell lung cancer, non-small cell lung cancer (“NSCLC”), vulval cancer, thyroid cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, hepatic carcinoma, anal carcinoma, penile carcinoma, as well as head and neck cancer. In yet other embodiments, the cancer is at least one selected from the group consisting of ALL, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL, Philadelphia chromosome positive CML, lymphoma, leukemia, multiple myeloma myeloproliferative diseases, large B cell lymphoma, and B cell Lymphoma.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary, and topical administration.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “effective amount,” “pharmaceutically effective amount,” and “therapeutically effective amount” refer to a nontoxic but sufficient amount of an agent to provide the desired biological result. That result may be reduction and/or alleviation of the signs, symptoms, or causes of a disease, or any other desired alteration of a biological system.

An appropriate therapeutic amount in any individual case may be determined by one of ordinary skill in the art using routine experimentation.

As used herein, the term “efficacy” refers to the maximal effect (Eₘₐₓ) achieved within an assay.

As used herein, the term “L” or “LINKER” refers to the linker.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, trifluoroacetic acid, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds of the invention include, for example, ammonium salts, metallic salts including alkali metal, alkaline earth metal and transition metal salts such as, for example, calcium, magnesium, potassium, sodium, and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine), and procaine. All of these salts may be prepared from the corresponding compound by reacting, for example, the appropriate acid or base with the compound.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

The terms “patient,” “subject,” or “individual” are used interchangeably herein, and refer to any animal, or cells thereof whether in vitro or in situ, amenable to the methods described herein. In a non-limiting embodiment, the patient, subject, or individual is a human.

As used herein, the term “potency” refers to the dose needed to produce half the maximal response (ED₅₀).

A “therapeutic” treatment is a treatment administered to a subject who exhibits signs of pathology, for the purpose of diminishing or eliminating those signs.

As used herein, the term “treatment” or “treating” is defined as the application or administration of a therapeutic agent, i.e., a compound of the invention (alone or in combination with another pharmaceutical agent), to a patient, or application or administration of a therapeutic agent to an isolated tissue or cell line from a patient (e.g., for diagnosis or ex vivo applications), who has a condition contemplated herein, a symptom of a condition contemplated herein or the potential to develop a condition contemplated herein, with the purpose to cure, heal, alleviate, relieve, alter, remedy, ameliorate, improve or affect a condition contemplated herein, the symptoms of a condition contemplated herein or the potential to develop a condition contemplated herein. Such treatments may be specifically tailored or modified, based on knowledge obtained from the field of pharmacogenomics.

As used herein, the term “alkyl,” by itself or as part of another substituent means, unless otherwise stated, a straight or branched chain hydrocarbon having the number of carbon atoms designated (i.e. C₁₋₆ means one to six carbon atoms) and including straight, branched chain, or cyclic substituent groups. Examples include methyl, ethyl, propyl, isopropyl, butyl, isobutyl, tert-butyl, pentyl, neopentyl, hexyl, and cyclopropylmethyl. Most preferred is (C₁-C₆)alkyl, particularly ethyl, methyl, isopropyl, isobutyl, n-pentyl, n-hexyl and cyclopropylmethyl.

As used herein, the term “substituted alkyl” means alkyl as defined above, substituted by one, two or three substituents selected from the group consisting of halogen, —OH, alkoxy, —NH₂, —N(CH₃)₂, —C(═O)OH, trifluoromethyl, —C≡N, —C(═O)O(C₁-C₄)alkyl, —C(═O)NH₂, —SO₂NH₂, —C(═NH)NH₂, and —NO₂, preferably containing one or two substituents selected from halogen, —OH, alkoxy, —NH₂, trifluoromethyl, —N(CH₃)₂, and —C(═O)OH, more preferably selected from halogen, alkoxy and —OH. Examples of substituted alkyls include, but are not limited to, 2,2-difluoropropyl, 2-carboxycyclopentyl and 3-chloropropyl.

The term “alkylene” refers to a diradical of an alkyl group. Exemplary alkylene groups include —CH₂—, —CH₂CH₂—, and —CH₂C(H)(CH₃)CH₂—. The term “—(C₀ alkylene)-” refers to a bond. Accordingly, the term “—(C₀₋₃ alkylene)-” encompasses a bond (i.e., C₀) and a —(C₁₋₃ alkylene) group.

As used herein, the term “haloalkyl” means alkyl as defined above, substituted by one, two or three substituents selected from the group consisting of F, Cl, Br, and I.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized or substituted. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, —NH—(CH₂)ₘ—OH (m=1-6), —N(CH₃)—(CH₂)ₘ—OH (m=1-6), —NH—(CH₂)ₘ—OCH₃ (m=1-6), and —CH₂CH₂—S(═O)—CH₃. Up to two heteroatoms may be consecutive, such as, for example, —CH₂—NH—OCH₃, or —CH₂—CH₂—S—S—CH₃.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred are (C₁-C₃) alkoxy, particularly ethoxy and methoxy. In certain embodiments, the heteroalkyl contains from 2 to 10 atoms selected from the group consisting of carbon and a heteroatom (e.g., O, N, or S). In certain embodiments, the heteroalkyl contains from 2 to 4, 2 to 6, 2 to 8, or 3 to 6 atoms selected from the group consisting of carbon and a heteroatom (e.g., O, N, or S).

As used herein, the term “cycloalkyl” refers to a mono cyclic or polycyclic non-aromatic radical, wherein each of the atoms forming the ring (i.e. skeletal atoms) is a carbon atom. In certain embodiments, the cycloalkyl group is saturated or partially unsaturated. In other embodiments, the cycloalkyl group is fused with an aromatic ring. Cycloalkyl groups include groups having from 3 to 10 ring atoms. Illustrative examples of cycloalkyl groups include, but are not limited to, the following moieties:

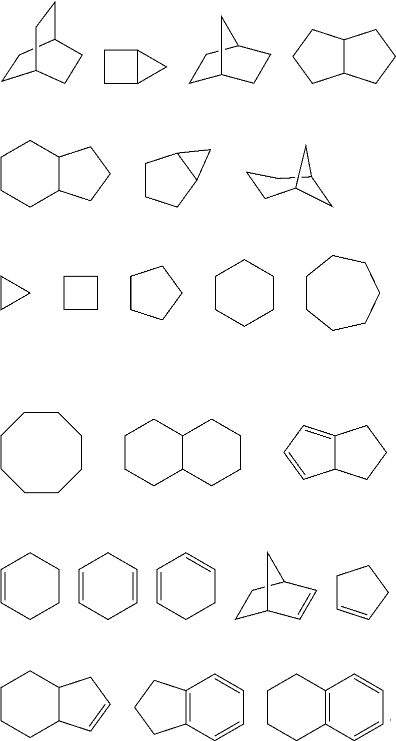

Monocyclic cycloalkyls include, but are not limited to, cyclopropyl, cyclobutyl, cyclopentyl, cyclohexyl, cycloheptyl, and cyclooctyl. Dicyclic cycloalkyls include, but are not limited to, tetrahydronaphthyl, indanyl, and tetrahydropentalene. Polycyclic cycloalkyls include adamantine and norbornane. The term cycloalkyl includes “unsaturated nonaromatic carbocyclyl” or “nonaromatic unsaturated carbocyclyl” groups, both of which refer to a nonaromatic carbocycle as defined herein, which contains at least one carbon carbon double bond or one carbon carbon triple bond.

As used herein, the term “aromatic” refers to a carbocycle or heterocycle with one or more polyunsaturated rings and having aromatic character, i.e. having (4n+2) delocalized it (pi) electrons, where n is an integer.

As used herein, the term “aryl,” employed alone or in combination with other terms, means, unless otherwise stated, a carbocyclic aromatic system containing one or more rings (typically one, two or three rings), wherein such rings may be attached together in a pendent manner, such as a biphenyl, or may be fused, such as naphthalene. Examples of aryl groups include phenyl, anthracyl, and naphthyl. Preferred examples are phenyl and naphthyl, most preferred is phenyl.

As used herein, the term “aryl-(C₁-C₃)alkyl” means a functional group wherein a one- to three-carbon alkylene chain is attached to an aryl group, e.g., —CH₂CH₂-phenyl. Preferred is aryl-CH₂— and aryl-CH(CH₃)—. The term “substituted aryl-(C₁-C₃)alkyl” means an aryl-(C₁-C₃)alkyl functional group in which the aryl group is substituted. Preferred is substituted aryl(CH₂)—. Similarly, the term “heteroaryl-(C₁-C₃)alkyl” means a functional group wherein a one to three carbon alkylene chain is attached to a heteroaryl group, e.g., —CH₂CH₂-pyridyl. Preferred is heteroaryl-(CH₂)—. The term “substituted heteroaryl-(C₁-C₃)alkyl” means a heteroaryl-(C₁-C₃)alkyl functional group in which the heteroaryl group is substituted. Preferred is substituted heteroaryl-(CH₂)—.

The term “carbocyclyl” refers to a saturated or unsaturated carbocyclic ring system containing one or more rings (typically one, two or three rings). In certain embodiments, the carbocyclyl is a 3-12 membered carbocyclic ring, a 3-8 membered carbocyclic ring, or a 3-6 membered carbocyclic ring.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

The term “heteroalkylene” refers to an alkylene group in which one or more carbon atoms has been replaced by a heteroatom (e.g., N, O, or S). Exemplary heteroalkylene groups include —CH₂O—, —CH₂OCH₂—, and —CH₂CH₂O—. The heteroalkylene group may contain, for example, from 2-4, 2-6, or 2-8 atoms selected from the group consisting of carbon and a heteroatom (e.g., N, O, or S).

As used herein, the term “heterocycloalkyl” or “heterocyclyl” refers to a heteroalicyclic group containing one to four ring heteroatoms each selected from O, S and N. In certain embodiments, each heterocycloalkyl group has from 4 to 10 atoms in its ring system, with the proviso that the ring of said group does not contain two adjacent O or S atoms. In other embodiments, the heterocycloalkyl group is fused with an aromatic ring. In certain embodiments, the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In certain embodiments, the heterocycle is a heteroaryl.

An example of a 3-membered heterocycloalkyl group includes, and is not limited to, aziridine. Examples of 4-membered heterocycloalkyl groups include, and are not limited to, azetidine and a beta lactam. Examples of 5-membered heterocycloalkyl groups include, and are not limited to, pyrrolidine, oxazolidine and thiazolidinedione. Examples of 6-membered heterocycloalkyl groups include, and are not limited to, piperidine, morpholine and piperazine. Other non-limiting examples of heterocycloalkyl groups are:

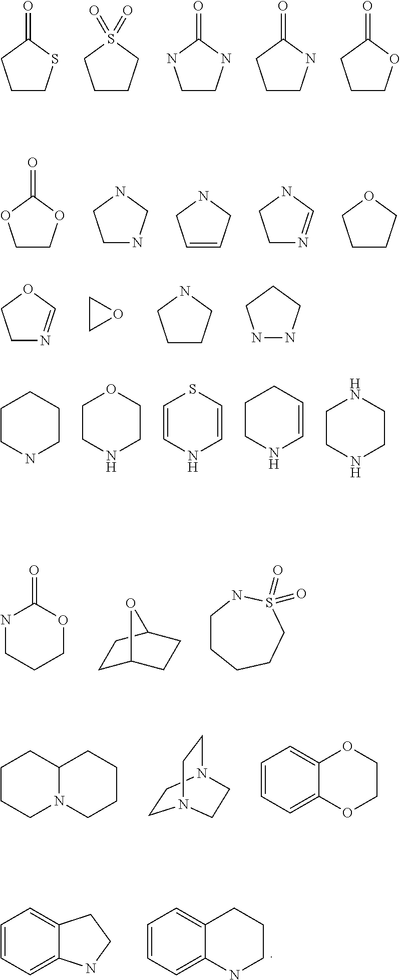

Examples of non-aromatic heterocycles include monocyclic groups such as aziridine, Examples of non-aromatic heterocycles include monocyclic groups such as aziridine, oxirane, thiirane, azetidine, oxetane, thietane, pyrrolidine, pyrroline, pyrazolidine, imidazoline, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin, and hexamethyleneoxide.

As used herein, the term “heteroaryl” or “heteroaromatic” refers to a heterocycle having aromatic character. A polycyclic heteroaryl may include one or more rings that are partially saturated. Examples include the following moieties:

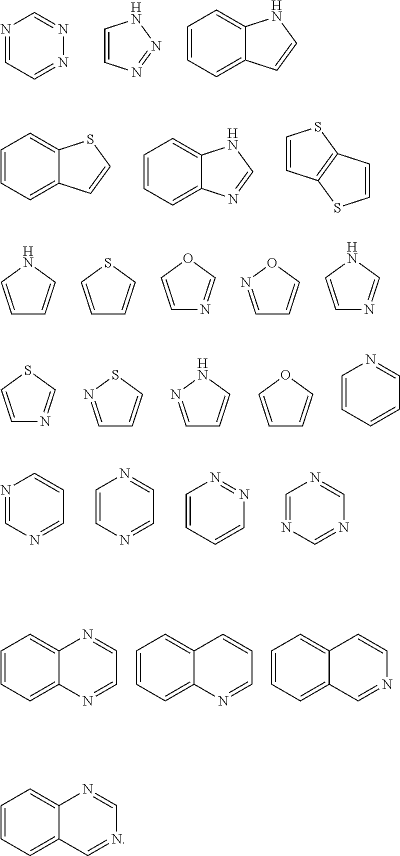

Examples of heteroaryl groups also include pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles and heteroaryls include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benzotriazolyl, thioxanthinyl, carbazolyl, carbolinyl, acridinyl, pyrrolizidinyl, and quinolizidinyl.

The term “heteroarylene” refers to a multi-valent (e.g., di-valent or trivalent) aromatic group that comprises at least one ring heteroatom. An exemplary “heteroarylene” is pyridinylene, which is a multi-valent radical of pyridine. For example, a divalent radical of pyridine is illustrated by the formula

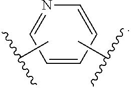

In certain embodiments, the “heteroarylene” is a divalent, 5-6 membered heteroaromatic group containing 1, 2, or 3 ring heteroatoms (e.g., O, N, or S).

The term “phenylene” refers to a multivalent radical (e.g., a divalent or trivalent radical) of benzene. To illustrate, a divalent radical of benzene is illustrated by the formula

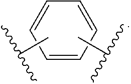

As used herein, the term “substituted” means that an atom or group of atoms has replaced hydrogen as the substituent attached to another group. The term “substituted” further refers to any level of substitution, namely mono-, di-, tri-, tetra-, or penta-substitution, where such substitution is permitted. The substituents are independently selected, and substitution may be at any chemically accessible position. In certain embodiments, the substituents vary in number between one and four. In other embodiments, the substituents vary in number between one and three. In yet other embodiments, the substituents vary in number between one and two.

As used herein, the term “optionally substituted” means that the referenced group may be substituted or unsubstituted. In certain embodiments, the referenced group is optionally substituted with zero substituents, i.e., the referenced group is unsubstituted. In other embodiments, the referenced group is optionally substituted with one or more additional group(s) individually and independently selected from groups described herein.

In certain embodiments, the substituents are independently selected from the group consisting of oxo, halogen, —CN, —NH₂, —OH, —NH(CH₃), —N(CH₃)₂, alkyl (including straight chain, branched and/or unsaturated alkyl), substituted or unsubstituted cycloalkyl, substituted or unsubstituted heterocycloalkyl, fluoro alkyl, substituted or unsubstituted heteroalkyl, substituted or unsubstituted alkoxy, fluoroalkoxy, —S-alkyl, S(═O)₂ alkyl, —C(═O)NH[substituted or unsubstituted alkyl, or substituted or unsubstituted phenyl], —C(═O)N[H or alkyl]₂, —OC(═O)N[substituted or unsubstituted alkyl]₂, —NHC(═O)NH [substituted or unsubstituted alkyl, or substituted or unsubstituted phenyl], —NHC(═O)alkyl, —N[substituted or unsubstituted alkyl]C(═O)[substituted or unsubstituted alkyl], —NHC(═O)[substituted or unsubstituted alkyl], —C(OH)[substituted or unsubstituted alkyl]₂, and —C(NH₂)[substituted or unsubstituted alkyl]₂. In other embodiments, by way of example, an optional substituent is selected from oxo, fluorine, chlorine, bromine, iodine, —CN, —NH₂, —OH, —NH(CH₃), —N(CH₃)₂, —CH₃, —CH₂CH₃, —CH(CH₃)₂, —CF₃, —CH₂CF₃, —OCH₃, —OCH₂CH₃, —OCH(CH₃)₂, —OCF₃, —OCH₂CF₃, —S(═O)₂—CH₃, —C(═O)NH₂, —C(═O)—NHCH₃, —NHC(═O)NHCH₃, —C(═O)CH₃, and —C(═O)OH. In yet one embodiment, the substituents are independently selected from the group consisting of C₁₋₆ alkyl, —OH, C₁₋₆ alkoxy, halo, amino, acetamido, oxo and nitro. In yet other embodiments, the substituents are independently selected from the group consisting of C₁₋₆ alkyl, C₁₋₆ alkoxy, halo, acetamido, and nitro. As used herein, where a substituent is an alkyl or alkoxy group, the carbon chain may be branched, straight or cyclic, with straight being preferred.

In certain embodiments, an optional substituent is selected from the group consisting of C₁-C₆ alkyl, C₃-C₈ cycloalkyl, phenyl, C₁-C₆ hydroxyalkyl, (C₁-C₆ alkoxy)-C₁-C₆ alkyl, C₁-C₆ haloalkyl, C₁-C₆ haloalkoxy, halogen, —CN, —OR^{b}, —N(R^{b})(R^{b}), —NO₂, —C(═O)N(R^{b})(R^{b}), —S(═O)₂N(R^{b})(R^{b}), acyl, and C₁-C₆ alkoxycarbonyl, wherein each occurrence of R^{b} is independently H, C₁-C₆ alkyl, or C₃-C₈ cycloalkyl, wherein in R^{b} the alkyl or cycloalkyl is optionally substituted with at least one selected from the group consisting of halogen, —OH, C₁-C₆ alkoxy, and heteroaryl; or substituents on two adjacent carbon atoms combine to form —O(CH₂)₁₋₃O—.

In certain embodiments, an optional substituent is selected from the group consisting of C₁-C₆ alkyl, C₃-C₈ cycloalkyl, phenyl, C₁-C₆ hydroxyalkyl, (C₁-C₆ alkoxy)-C₁-C₆ alkyl, C₁-C₆ haloalkyl, C₁-C₆ haloalkoxy, halogen, —OR^{b}, and —C(═O)N(R^{b})(R^{b}), wherein each occurrence of R^{b} is independently H, C₁-C₆ alkyl, or C₃-C₈ cycloalkyl, wherein in R^{b} the alkyl or cycloalkyl is optionally substituted with at least one selected from the group consisting of halogen, —OH, C₁-C₆ alkoxy, and heteroaryl; or substituents on two adjacent carbon atoms combine to form —O(CH₂)₁₋₃O—.

In certain embodiments, an optional substituent is selected from the group consisting of C₁-C₆ alkyl, —OH, C₁-C₃ haloalkyl, C₁-C₆ alkoxy, C₃-C₈ cycloalkyl, C₃-C₈ cycloalkoxy, halo, and —CN.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6, and so forth, as well as individual numbers within that range, for example, 1, 2, 2.7, 3, 4, 5, 5.3, and 6. This applies regardless of the breadth of the range.

Throughout this disclosure, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

### Compounds

In certain embodiments, the compound of the invention comprises, and/or has, the formula:

(protein phosphatase ligand)-LINKER-(target protein ligand) (I);

wherein the protein phosphatase ligand binds to a protein phosphatase, such that the protein phosphatase ligand does not significantly inhibit the phosphatase activity of the protein phosphatase;

wherein the target protein ligand binds to a target protein;

wherein the LINKER is selected such that it allows for the compound to bind simultaneously to the protein phosphatase and the target protein;

wherein, when the compound is simultaneously bound to the protein phosphatase and the target protein, the protein phosphatase is capable of dephosphorylating the target protein;

or a salt, solvate, prodrug, isotopically labelled derivative, stereoisomer, tautomer, or geometric isomer thereof, and any mixtures thereof.

A non-limiting embodiment of a compound of the invention (depicted therein as

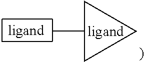

is illustrated in Scheme I:

The protein phosphatase ligand, LINKER, and target protein ligand are described in more detail herein.

In certain embodiments, a compound of the invention is represented by Formula (I-A), or a salt or solvate thereof:

(protein phosphatase ligand)-LINKER-(target protein ligand) (I-A);

wherein:

the protein phosphatase ligand binds to a protein phosphatase;

the target protein ligand binds to a target protein; and

the LINKER is a bond or a group that allows the compound to bind to the protein phosphatase and the target protein.

In certain embodiments, in connection with the compound of Formula (I-A), the protein phosphatase ligand does not significantly inhibit phosphatase activity of the protein phosphatase.

Protein Phosphatase Ligand

In one aspect, the compounds of the invention comprise a ligand that binds to a protein phosphatase (“protein phosphatase ligand”). Any known protein phosphatase ligand is useful within the invention, as long as the protein phosphatase ligand does not bind to the active site of the protein phosphatase and/or does not inhibit its phosphatase activity.

In certain embodiments, the phosphatases contemplated within the invention include, but are not limited to, protein phosphatase 1 (PP1), protein phosphatase 2 (PP2), protein phosphatase 2A (PP2A), protein phosphatase 2B (PP2B), protein phosphatase 2C (PP2C), any of PTPRA through PTPRZ, and any of dual specific phosphatases DUSP1 through DUSP27.

In certain embodiments, the phosphatases contemplated within the invention include, but are not limited to, protein phosphatase 1 (PP1), protein phosphatase 2A (PP2A), protein phosphatase 2B (PP2B), protein phosphatase 2C (PP2C), any of PTPRA through PTPRZ, and any of dual specific phosphatases DUSP1 through DUSP27.

In certain embodiments, the phosphatases contemplated within the invention include, but are not limited to, CDC25A, CDC25B, CDC25C, ACP1, and Eya1 through Eya4.

In certain embodiments, the protein phosphatase is protein phosphatase 1 (PP1). A non-limiting example of a protein phosphatase ligand contemplated within the invention includes, but is not limited to, a peptide comprising the sequence Arg Val Xaa Phe (also known as RVXaaF; wherein Xaa is any natural or non-natural amino acid; SEQ ID NO: 1). In other embodiments, the protein phosphatase ligand comprises the sequence RRKRPKRKRKNARVTF(Xaa¹⁷)EAAEII (SEQ ID NO:2), wherein (Xaa¹⁷) is L-4-benzoylphenylalanine. In yet other embodiments, the protein phosphatase ligand comprises the sequence RRKRPKRKRKNARVTFFEAAEII (SEQ ID NO:3).

In certain embodiments, the protein phosphatase is protein phosphatase 2A (PP2A). A non-limiting example of protein phosphatase ligands contemplated within the invention includes, but is not limited to, a peptide comprising the sequence Leu Ser Pro Ile Xaa Glu (also known as LSPIXaaE; wherein Xaa is any natural or non-natural amino acid; SEQ ID NO:4). In other embodiments, the protein phosphatase ligand comprises the sequence GLLSPIPERRRRRRRR (SEQ ID NO:5).

In certain embodiments, the protein phosphatase ligand binds to protein phosphatase 2A (PP2A), protein phosphatase 2B (PP2B), or protein phosphatase 2C (PP2C).

In certain embodiments, the protein phosphatase ligand binds to protein phosphatase 1 (PP1). In certain embodiments, the protein phosphatase ligand binds to protein phosphatase 2A (PP2A). In certain embodiments, the protein phosphatase ligand binds to protein phosphatase 2B (PP2B). In certain embodiments, the protein phosphatase ligand binds to protein phosphatase 2C (PP2C). In certain embodiments, the protein phosphatase ligand binds to one of PTPRA through PTPRZ. In certain embodiments, the protein phosphatase ligand binds to one of dual specific phosphatases DUSP1 through DUSP27.

In certain embodiments, the protein phosphatase ligand binds to CDC25A. In certain embodiments, the protein phosphatase ligand binds to CDC25B. In certain embodiments, the protein phosphatase ligand binds to CDC25C. In certain embodiments, the protein phosphatase ligand binds to ACP1. In certain embodiments, the protein phosphatase ligand binds to one of Eya1 through Eya4.

In certain embodiments, the protein phosphatase ligand is a small organic molecule, such as having a molecular weight of less than 1500 Da, 1200 Da, 1000 Da, 800 Da, 600 Da, 400 Da, 300 Da, 200 Da, 150 Da, or 100 Da.

In certain embodiments, the protein phosphatase ligand component of Formula (I) has the following formula:

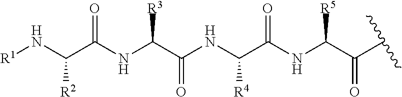

wherein:
R¹ is hydrogen or an optionally substituted group selected from —C(═O)(C₁-C₈ alkyl), —C(═O)(C₃-C₈ cycloalkyl), —C(═O)(C₀-C₃ alkylene)-aryl, and C₁-C₈ alkyl;
R² is optionally substituted —(C₁-C₈ alkylene)-N(H)—C(═NH)NH₂;
R³ is optionally substituted C₁-C₈ alkyl;
R⁴ is optionally substituted C₁-C₈ hydroxyalkyl; and
R⁵ is optionally substituted —(C₀-C₃ alkylene)-aryl or optionally substituted —(C₀-C₃ alkylene)-heteroaryl.

In certain embodiments, the protein phosphatase ligand component of Formula (I) has the following formula:

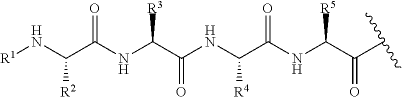

wherein:
R¹ is hydrogen, —C(═O)(C₁-C₈ alkyl), —C(═O)(C₃-C₈ cycloalkyl), or —C(═O)(C₀-C₃ alkylene)-aryl;
R² is —(C₁-C₆ alkylene)-N(H)—C(═NH)NH₂;
R³ is C₁-C₆ alkyl;
R⁴ is C₁-C₆ hydroxyalkyl; and
R⁵ is —(C₀-C₃ alkylene)-aryl.

In certain embodiments, the protein phosphatase ligand component of Formula (I) has the following formula:

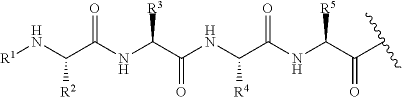

wherein:
R¹ is hydrogen or —C(═O)(C₁-C₈ alkyl);
R² is —(C₁-C₈ alkylene)-N(H)—C(═NH)NH₂;
R³ is C₁-C₈ alkyl;
R⁴ is C₁-C₈ hydroxyalkyl; and
R⁵ is —(C₀-C₃ alkylene)-aryl.

In certain embodiments, the protein phosphatase ligand component of Formula (I) has the following formula:

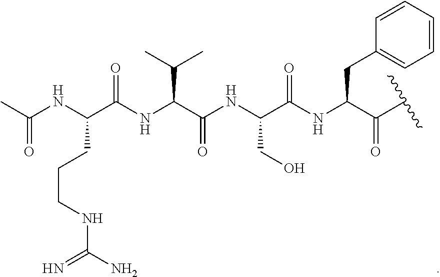 [SMILES: *C(=O)[C@H](Cc1ccccc1)NC(=O)[C@H](CO)NC(=O)[C@@H](NC(=O)[C@H](CCCNC(=N)N)NC(C)=O)C(C)C]

In certain embodiments, the protein phosphatase ligand component of Formula (I) has the following formula:

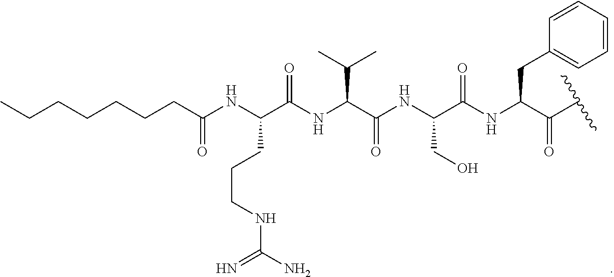 [SMILES: *C(=O)[C@H](Cc1ccccc1)NC(=O)[C@H](CO)NC(=O)[C@@H](NC(=O)[C@H](CCCNC(=N)N)NC(=O)CCCCCCC)C(C)C]

In certain embodiments, the protein phosphatase ligand component of Formula (I) has the following formula:

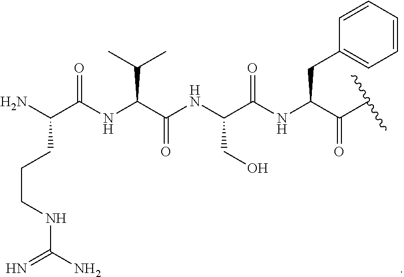 [SMILES: *C(=O)[C@H](Cc1ccccc1)NC(=O)[C@H](CO)NC(=O)[C@@H](NC(=O)[C@@H](N)CCCNC(=N)N)C(C)C]

In certain embodiments, the protein phosphatase ligand component of Formula (I) has the following formula:

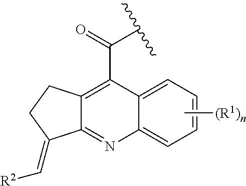

wherein:
R¹ represents independently for each occurrence halogen, C₁-C₆ alkyl, C₁-C₆ haloalkyl, C₃-C₆ cycloalkyl, hydroxyl, C₁-C₆ alkoxy, or cyano;
R² is optionally substituted —(C₀-C₃ alkylene)-aryl or optionally substituted —(C₀-C₃ alkylene)-heteroaryl; and
n is 0, 1, 2, or 3.

In certain embodiments, the protein phosphatase ligand component of Formula (I) has the following formula:

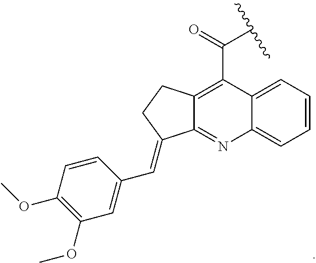 [SMILES: *C(=O)c1c2c(nc3ccccc13)/C(=C/c1ccc(OC)c(OC)c1)CC2]

In certain embodiments, the protein phosphatase ligand component of Formula (I) has the following formula:

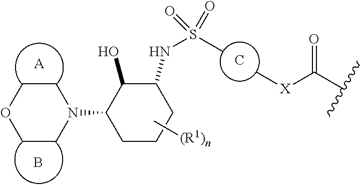

wherein:
each of A and B is independently an optionally substituted 6-membered carbocyclic aromatic ring or an optionally substituted 5-6 membered heteroaromatic ring;
C is an optionally substituted phenylene or an optionally substituted 5-6 membered heteroarylene;
X is a bond, —O—, —N(R²)—, or optionally substituted 2-5 membered heteroalkylene;
R¹ represents independently for each occurrence halogen, C₁-C₆ alkyl, C₁-C₆ haloalkyl, C₃-C₆ cycloalkyl, hydroxyl, C₁-C₆ alkoxy, or cyano;
R² is hydrogen or optionally substituted C₁-C₆ alkyl; and
n is 0, 1, 2, or 3.

In certain embodiments, the protein phosphatase ligand component of Formula (I) has the following formula:

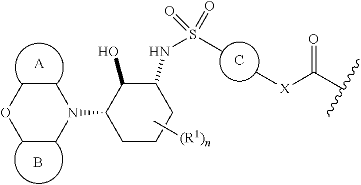

wherein:
each of A and B is a 6-membered carbocyclic aromatic ring;
C is phenylene;
X is —N(R²)—;
R¹ represents independently for each occurrence halogen, C₁-C₆ alkyl, or C₁-C₆ haloalkyl;
R² is hydrogen or C₁-C₆ alkyl; and
n is 0, 1, or 2.

In certain embodiments, the protein phosphatase ligand component of Formula (I) has the following formula:

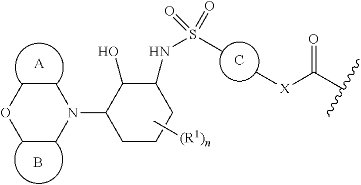

wherein:
C is an optionally substituted phenylene or an optionally substituted pyridinylene;
X is a bond, —O—, —N(R²)—, or 2-5 membered heteroalkylene optionally substituted with 1-3 fluoro;
R¹ represents independently for each occurrence halogen, C₁-C₆ alkyl, C₁-C₆ haloalkyl, C₃-C₆ cycloalkyl, hydroxyl, C₁-C₆ alkoxy, or cyano;
R² is hydrogen or optionally substituted C₁-C₆ alkyl; and
n is 0, 1, or 2.

In certain embodiments, the protein phosphatase ligand component of Formula (I) has the following formula:

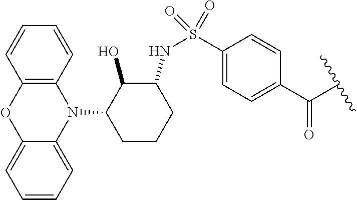 [SMILES: *C(=O)c1ccc(S(=O)(=O)N[C@@H]2CCC[C@H](N3c4ccccc4Oc4ccccc43)[C@H]2O)cc1]

In certain embodiments, the protein phosphatase ligand component of Formula (I) has the following formula:

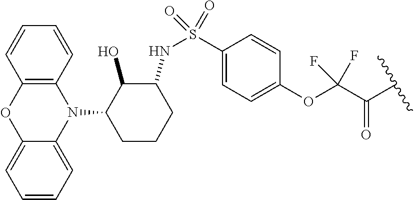 [SMILES: *C(=O)C(F)(F)Oc1ccc(S(=O)(=O)N[C@@H]2CCC[C@H](N3c4ccccc4Oc4ccccc43)[C@H]2O)cc1]

In certain embodiments, the protein phosphatase ligand component of Formula (I) has the following formula:

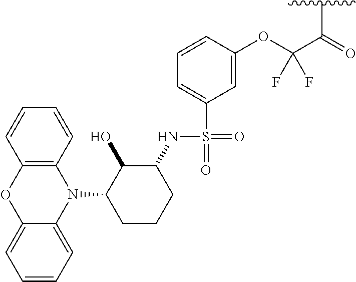 [SMILES: *C(=O)C(F)(F)Oc1cccc(S(=O)(=O)N[C@@H]2CCC[C@H](N3c4ccccc4Oc4ccccc43)[C@H]2O)c1]

In certain embodiments, the protein phosphatase ligand component of Formula (I) has the following formula:

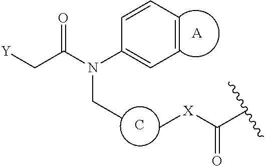

wherein:
A is an optionally substituted 5-7 membered partially unsaturated or aromatic heterocyclic ring;
C is an optionally substituted phenylene or an optionally substituted 5-6 membered heteroarylene;
X is a bond, —O—, —N(R)—, or 2-5 membered heteroalkylene;
Y is chloro or bromo; and
R is hydrogen or optionally substituted C₁-C₆ alkyl.

In certain embodiments, the protein phosphatase ligand component of Formula (I) has the following formula:

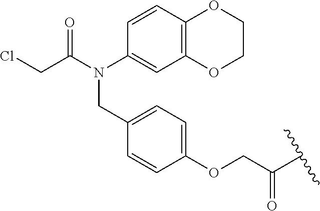 [SMILES: *C(=O)COc1ccc(CN(C(=O)CCl)c2ccc3c(c2)OCCO3)cc1]

In certain embodiments, the protein phosphatase ligand component of Formula (I) has the following formula:

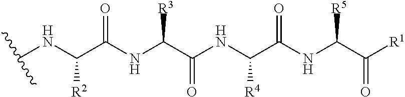

wherein:
R¹ is —N(H)R⁶, —N(R⁶)(R⁷), —NH₂, —OH, or —OR⁶;
R² is optionally substituted —(C₁-C₈ alkylene)-N(H)—C(═NH)NH₂;
R³ is optionally substituted C₁-C₈ alkyl;
R⁴ is optionally substituted C₁-C₈ hydroxyalkyl;
R⁵ is optionally substituted —(C₀-C₃ alkylene)-aryl or optionally substituted —(C₀-C₃ alkylene)-heteroaryl; and
R⁶ and R⁷ are independently optionally substituted C₁-C₈ alkyl, or R⁶ and R⁷ are taken together with the nitrogen atom to which they are attached to form an optionally substituted 4-8 membered heterocyclic ring.

In certain embodiments, the protein phosphatase ligand component of Formula (I) has the following formula:

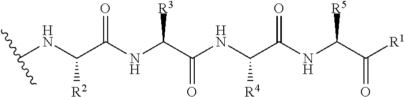

wherein:
R¹ is —N(H)R⁶, —N(R⁶)(R⁷), —NH₂, —OH, or —OR⁶;
R² is —(C₁-C₆ alkylene)-N(H)—C(═NH)NH₂;
R³ is C₁-C₆ alkyl;
R⁴ is C₁-C₆ hydroxyalkyl;
R⁵ is —(C₀-C₃ alkylene)-aryl; and
R⁶ and R⁷ are independently C₁-C₈ alkyl, or R⁶ and R⁷ are taken together with the nitrogen atom to which they are attached to form a 4-8 membered heterocyclic ring.

In certain embodiments, the protein phosphatase ligand component of Formula (I) has the following formula:

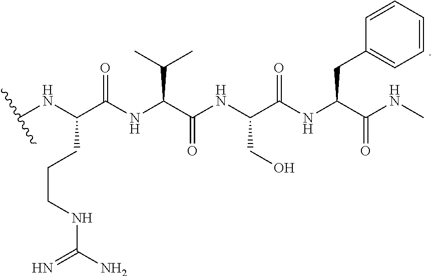 [SMILES: *N[C@@H](CCCNC(=N)N)C(=O)N[C@H](C(=O)N[C@@H](CO)C(=O)N[C@@H](Cc1ccccc1)C(=O)NC)C(C)C]

In certain embodiments, the protein phosphatase ligand component of Formula (I) has the following formula:

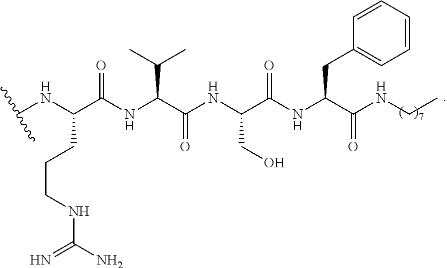

In certain embodiments, the protein phosphatase ligand component of Formula (I) has the following formula:

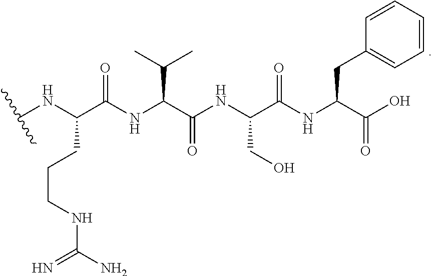 [SMILES: *N[C@@H](CCCNC(=N)N)C(=O)N[C@H](C(=O)N[C@@H](CO)C(=O)N[C@@H](Cc1ccccc1)C(=O)O)C(C)C]

In certain embodiments, the protein phosphatase ligand component of Formula (I) has the following formula:

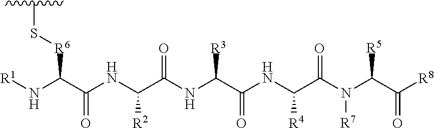

wherein:
R¹ is an optionally substituted group selected from —C(═O)(C₁-C₈ alkyl), —C(═O)(C₃-C₈
cycloalkyl), —C(═O)(C₀-C₃ alkylene)-aryl, and C₁-C₈ alkyl;
R² is optionally substituted —(C₁-C₈ alkylene)-N(H)—C(═NH)NH₂;
R³ is optionally substituted C₁-C₈ alkyl;
R⁴ is optionally substituted C₁-C₈ hydroxyalkyl;
R⁵ is an optionally substituted group selected from C₁-C₈ alkyl, —(C₀-C₃ alkylene)-aryl, and —(C₀-C₃ alkylene)-heteroaryl;
R⁶ is optionally substituted C₁-C₈ alkylene;
R⁷ is hydrogen or optionally substituted C₁-C₈ alkyl;
R⁸ is —N(H)R⁹, —N(R⁹)(R¹⁰), —NH₂, —OH, or —OR⁹; and
R⁹ and R¹⁰ are independently optionally substituted C₁-C₈ alkyl, or R⁹ and R¹⁰ are taken together with the nitrogen atom to which they are attached to form an optionally substituted 4-8 membered heterocyclic ring.

In certain embodiments, the protein phosphatase ligand component of Formula (I) has the following formula:

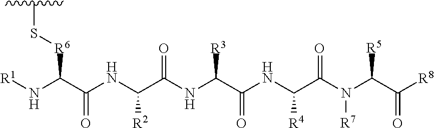

wherein:
R¹ is —C(═O)(C₁-C₈ alkyl), —C(═O)(C₃-C₈ cycloalkyl), or —C(═O)(C₀-C₃ alkylene)-aryl;
R² is —(C₁₋C₆ alkylene)-N(H)—C(═NH)NH₂;
R³ is C₁-C₆ alkyl;
R⁴ is C₁-C₆ hydroxyalkyl or —(C₀-C₃ alkylene)-heteroaryl;
R⁵ is C₁-C₆ alkyl or —(C₀-C₃ alkylene)-aryl;
R⁶ is C₁-C₆ alkylene;
R⁷ is hydrogen or C₁-C₆ alkyl;
R⁸ is —N(H)R⁹, —N(R⁹)(R¹⁰), —NH₂, —OH, or —OR⁹; and
R⁹ and R¹⁰ are independently C₁-C₈ alkyl, or R⁹ and R¹⁰ are taken together with the nitrogen atom to which they are attached to form a 4-8 membered heterocyclic ring.

In certain embodiments, the protein phosphatase ligand component of Formula (I) has the following formula:

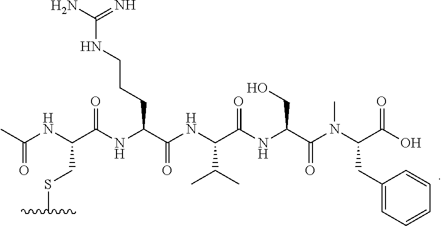 [SMILES: *SC[C@H](NC(C)=O)C(=O)N[C@@H](CCCNC(=N)N)C(=O)N[C@H](C(=O)N[C@@H](CO)C(=O)N(C)[C@@H](Cc1ccccc1)C(=O)O)C(C)C]

In certain embodiments, the protein phosphatase ligand component of Formula (I) has the following formula:

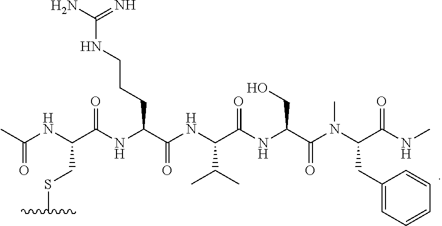 [SMILES: *SC[C@H](NC(C)=O)C(=O)N[C@@H](CCCNC(=N)N)C(=O)N[C@H](C(=O)N[C@@H](CO)C(=O)N(C)[C@@H](Cc1ccccc1)C(=O)NC)C(C)C]

In certain embodiments, the protein phosphatase ligand component of Formula (I) has the following formula:

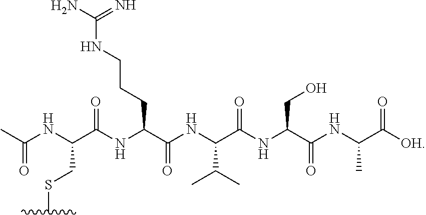 [SMILES: *SC[C@H](NC(C)=O)C(=O)N[C@@H](CCCNC(=N)N)C(=O)N[C@H](C(=O)N[C@@H](CO)C(=O)N[C@@H](C)C(=O)O)C(C)C]

In certain embodiments, the protein phosphatase ligand component of Formula (I) has the following formula:

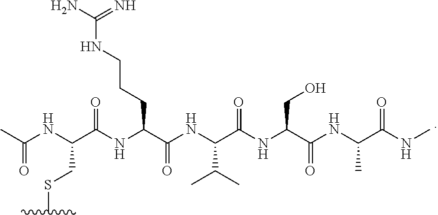 [SMILES: *SC[C@H](NC(C)=O)C(=O)N[C@@H](CCCNC(=N)N)C(=O)N[C@H](C(=O)N[C@@H](CO)C(=O)N[C@@H](C)C(=O)NC)C(C)C]

In certain embodiments, the protein phosphatase ligand component of Formula (I) has the following formula:

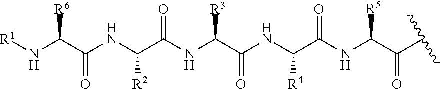

wherein:
R¹ is hydrogen or an optionally substituted group selected from —C(═O)(C₁-C₈ alkyl), —C(═O)(C₃-C₈ cycloalkyl), —C(═O)(C₀-C₃ alkylene)-aryl, and C₁-C₈ alkyl;
R² is optionally substituted —(C₁-C₈ alkylene)-N(H)—C(═NH)NH₂;
R³ is optionally substituted C₁-C₈ alkyl;
R⁴ is optionally substituted C₁-C₈ hydroxyalkyl or optionally substituted —(C₀-C₃ alkylene)-heteroaryl;
R⁵ is optionally substituted —(C₀-C₃ alkylene)-aryl or optionally substituted —(C₀-C₃ alkylene)-heteroaryl; and
R⁶ is optionally substituted —(C₁-C₈ alkylene)-NH₂.

In certain embodiments, the protein phosphatase ligand component of Formula (I) has the following formula:

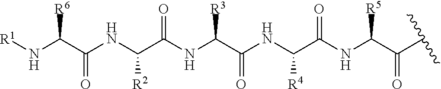

wherein:
R¹ is hydrogen, —C(═O)(C₁-C₈ alkyl), —C(═O)(C₃-C₈ cycloalkyl), or —C(═O)(C₀-C₃ alkylene)-aryl;
R² is —(C₁-C₆ alkylene)-N(H)—C(═NH)NH₂;
R³ is C₁-C₆ alkyl;
R⁴ is C₁-C₆ hydroxyalkyl or —(C₀-C₃ alkylene)-heteroaryl;
R⁵ is —(C₀-C₃ alkylene)-aryl; and
R⁶ is —(C₁-C₆ alkylene)-NH₂.

In certain embodiments, the protein phosphatase ligand component of Formula (I) has the following formula:

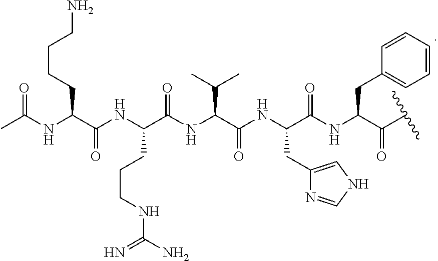 [SMILES: *C(=O)[C@H](Cc1ccccc1)NC(=O)[C@H](Cc1c[nH]cn1)NC(=O)[C@@H](NC(=O)[C@H](CCCNC(=N)N)NC(=O)[C@H](CCCCN)NC(C)=O)C(C)C]

In certain embodiments, the protein phosphatase ligand component of Formula (I) has the following formula:

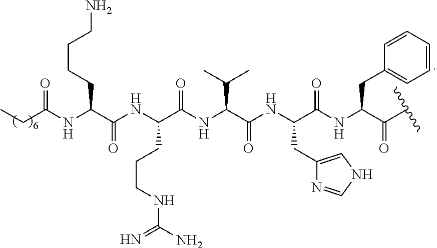

In certain embodiments, the protein phosphatase ligand component of Formula (I) has the following formula:

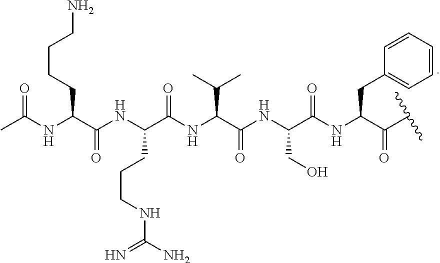 [SMILES: *C(=O)[C@H](Cc1ccccc1)NC(=O)[C@H](CO)NC(=O)[C@@H](NC(=O)[C@H](CCCNC(=N)N)NC(=O)[C@H](CCCCN)NC(C)=O)C(C)C]

In certain embodiments, the protein phosphatase ligand component of Formula (I) has the following formula:

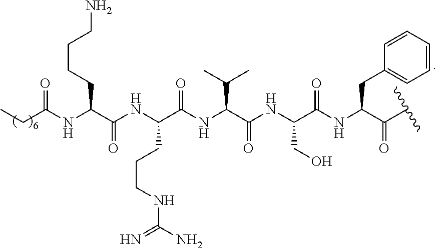

In certain embodiments, the protein phosphatase ligand component of Formula (I) has the following formula:

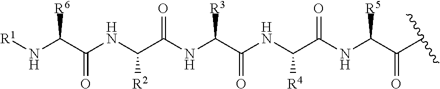

wherein:
R¹ is hydrogen or an optionally substituted group selected from —C(═O)(C₁-C₈ alkyl), —C(═O)(C₃-C₈ cycloalkyl), —C(═O)(C₀-C₃ alkylene)-aryl, and C₁-C₈ alkyl;
R² is optionally substituted C₁-C₈ hydroxyalkyl;
R³ is optionally substituted C₁-C₈ alkyl;
R⁴ is optionally substituted C₁-C₈ hydroxyalkyl;
R⁵ is optionally substituted —(C₀-C₃ alkylene)-aryl or optionally substituted —(C₀-C₃ alkylene)-heteroaryl; and
R⁶ is optionally substituted —(C₁-C₈ alkylene)-NH₂.

In certain embodiments, the protein phosphatase ligand component of Formula (I) has the following formula:

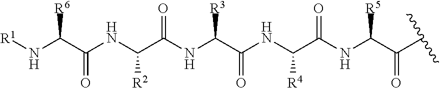

wherein:
R¹ is hydrogen, —C(═O)(C₁-C₈ alkyl), —C(═O)(C₃-C₈ cycloalkyl), or —C(═O)(C₀-C₃ alkylene)-aryl;
R² is C₁-C₆ hydroxyalkyl;
R³ is C₁-C₆ alkyl;
R⁴ is C₁-C₆ hydroxyalkyl;
R⁵ is —(C₀-C₃ alkylene)-heteroaryl; and
R⁶ is —(C₁-C₆ alkylene)-NH₂.

In certain embodiments, the protein phosphatase ligand component of Formula (I) has the following formula:

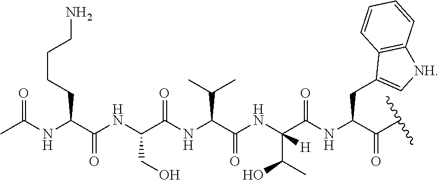 [SMILES: *C(=O)[C@H](Cc1c[nH]c2ccccc12)NC(=O)[C@@H](NC(=O)[C@@H](NC(=O)[C@H](CO)NC(=O)[C@H](CCCCN)NC(C)=O)C(C)C)[C@@H](C)O]

In certain embodiments, the protein phosphatase ligand component of Formula (I) has the following formula:

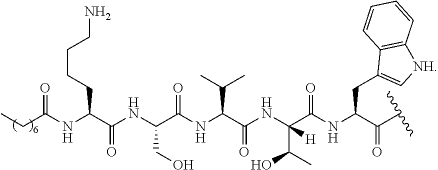

In certain embodiments, the protein phosphatase ligand component of Formula (I) has the following formula:

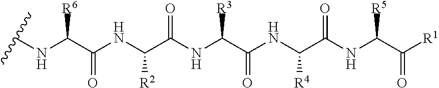

wherein:
R¹ is —N(H)R⁷, —N(R⁷)(R⁸), —NH₂, —OH, or —OR⁷;
R² is optionally substituted —(C₁-C₈ alkylene)-N(H)—C(═NH)NH₂;
R³ is optionally substituted C₁-C₈ alkyl;
R⁴ is optionally substituted C₁-C₈ hydroxyalkyl or optionally substituted —(C₀-C₃ alkylene)-heteroaryl;
R⁵ is optionally substituted —(C₀-C₃ alkylene)-aryl or optionally substituted —(C₀-C₃ alkylene)-heteroaryl;
R⁶ is optionally substituted —(C₁-C₈ alkylene)-NH₂; and
R⁷ and R⁸ are independently optionally substituted C₁-C₈ alkyl, or R⁷ and R⁸ are taken together with the nitrogen atom to which they are attached to form an optionally substituted 4-8 membered heterocyclic ring.

In certain embodiments, the protein phosphatase ligand component of Formula (I) has the following formula:

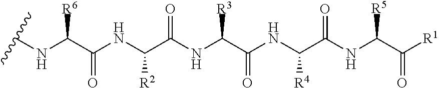

wherein:
R¹ is —N(H)R⁷, —N(R⁷)(R⁸), —NH₂, —OH, or —OR⁷;
R² is —(C₁-C₆ alkylene)-N(H)—C(═NH)NH₂;
R³ is C₁-C₆ alkyl;
R⁴ is C₁-C₆ hydroxyalkyl or —(C₀-C₃ alkylene)-heteroaryl;
R⁵ is —(C₀-C₃ alkylene)-aryl;
R⁶ is —(C₁-C₆ alkylene)-NH₂; and
R⁷ and R⁸ are independently C₁-C₈ alkyl, or R⁷ and R⁸ are taken together with the nitrogen atom to which they are attached to form a 4-8 membered heterocyclic ring.

In certain embodiments, the protein phosphatase ligand component of Formula (I) has the following formula:

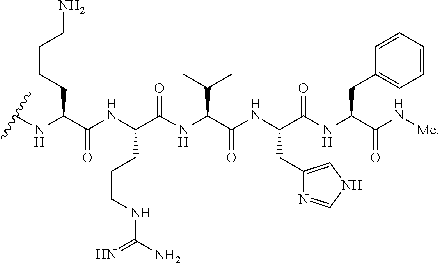 [SMILES: *N[C@@H](CCCCN)C(=O)N[C@@H](CCCNC(=N)N)C(=O)N[C@H](C(=O)N[C@@H](Cc1c[nH]cn1)C(=O)N[C@@H](Cc1ccccc1)C(=O)NC)C(C)C]

In certain embodiments, the protein phosphatase ligand component of Formula (I) has the following formula:

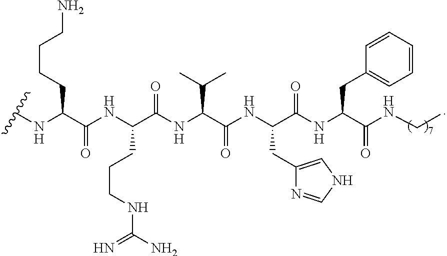

In certain embodiments, the protein phosphatase ligand component of Formula (I) has the following formula:

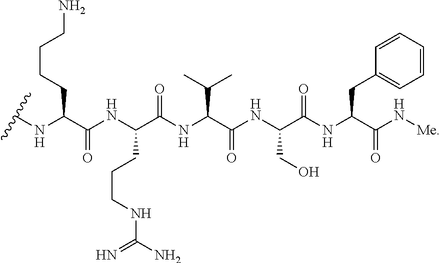 [SMILES: *N[C@@H](CCCCN)C(=O)N[C@@H](CCCNC(=N)N)C(=O)N[C@H](C(=O)N[C@@H](CO)C(=O)N[C@@H](Cc1ccccc1)C(=O)NC)C(C)C]

In certain embodiments, the protein phosphatase ligand component of Formula (I) has the following formula:

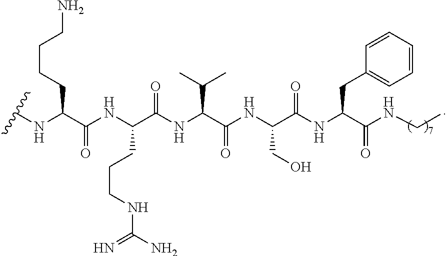

In certain embodiments, the protein phosphatase ligand component of Formula (I) has the following formula:

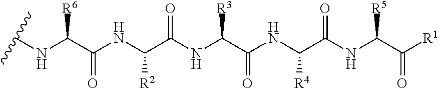

wherein:
R¹ is —N(H)R⁷, —N(R⁷)(R⁸), —NH₂, —OH, or —OR⁷;
R² is optionally substituted C₁-C₈ hydroxyalkyl;
R³ is optionally substituted C₁-C₈ alkyl;
R⁴ is optionally substituted C₁-C₈ hydroxyalkyl;
R⁵ is optionally substituted —(C₀-C₃ alkylene)-aryl or optionally substituted —(C₀-C₃ alkylene)-heteroaryl;
R⁶ is optionally substituted —(C₁-C₈ alkylene)-NH₂; and
R⁷ and R⁸ are independently optionally substituted C₁-C₈ alkyl, or R⁷ and R⁸ are taken together with the nitrogen atom to which they are attached to form an optionally substituted 4-8 membered heterocyclic ring.

In certain embodiments, the protein phosphatase ligand component of Formula (I) has the following formula:

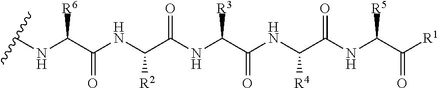

wherein:
R¹ is —N(H)R⁷, —N(R⁷)(R⁸), —NH₂, —OH, or —OR⁷;
R² is C₁-C₆ hydroxyalkyl;
R³ is C₁-C₆ alkyl;
R⁴ is C₁-C₆ hydroxyalkyl;
R⁵ is —(C₀-C₃ alkylene)-heteroaryl;
R⁶ is —(C₁-C₆ alkylene)-NH₂; and
R⁷ and R⁸ are independently C₁-C₈ alkyl, or R⁷ and R⁸ are taken together with the nitrogen atom to which they are attached to form a 4-8 membered heterocyclic ring.

In certain embodiments, the protein phosphatase ligand component of Formula (I) has the following formula:

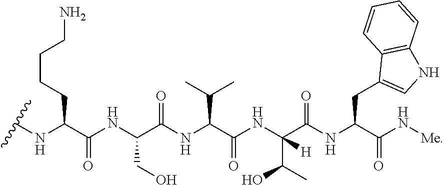 [SMILES: *N[C@@H](CCCCN)C(=O)N[C@@H](CO)C(=O)N[C@H](C(=O)N[C@H](C(=O)N[C@@H](Cc1c[nH]c2ccccc12)C(=O)NC)[C@@H](C)O)C(C)C]

In certain embodiments, the protein phosphatase ligand component of Formula (I) has the following formula:

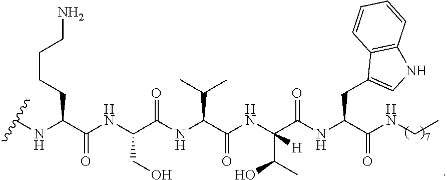

In certain embodiments, the protein phosphatase ligand component of Formula (I) is one of the following:

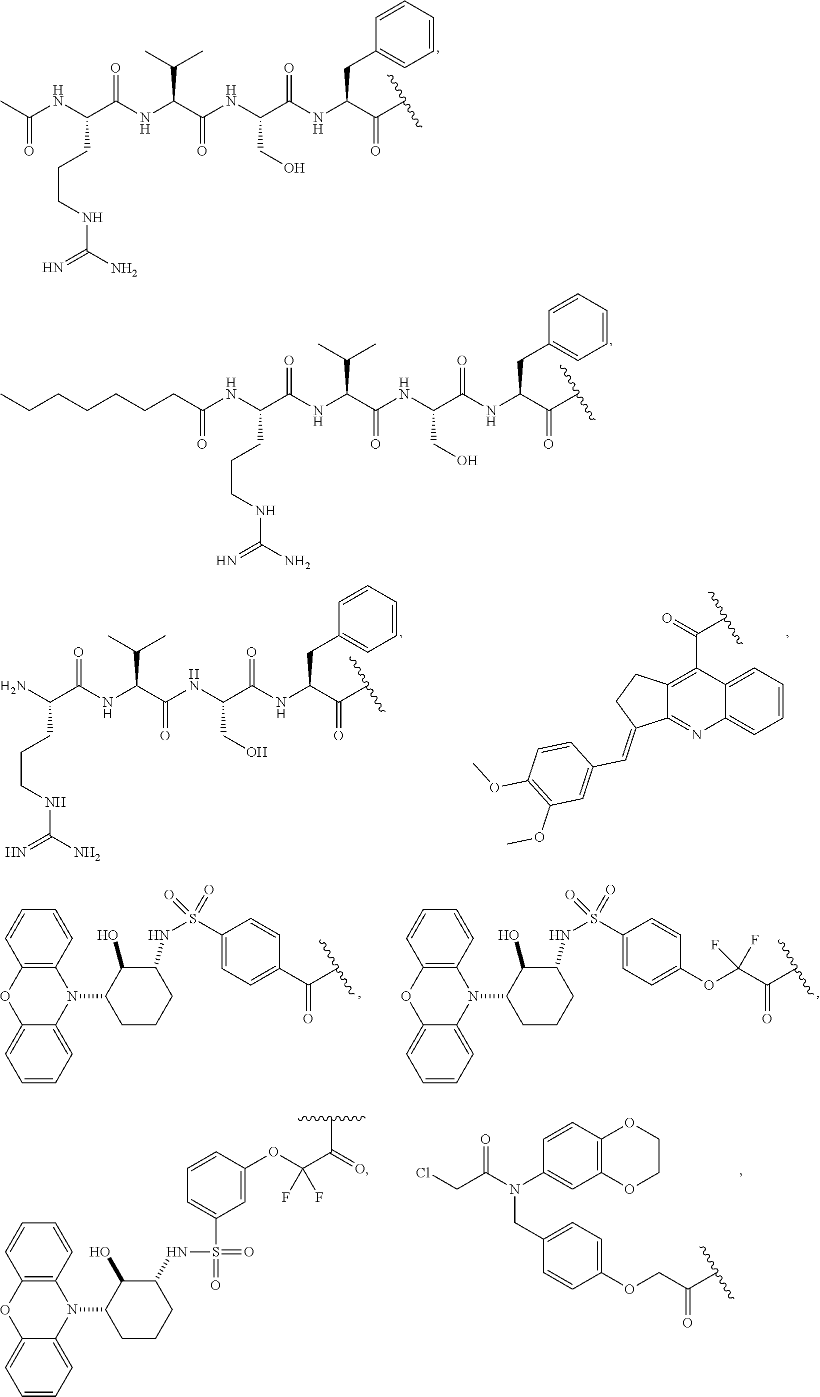

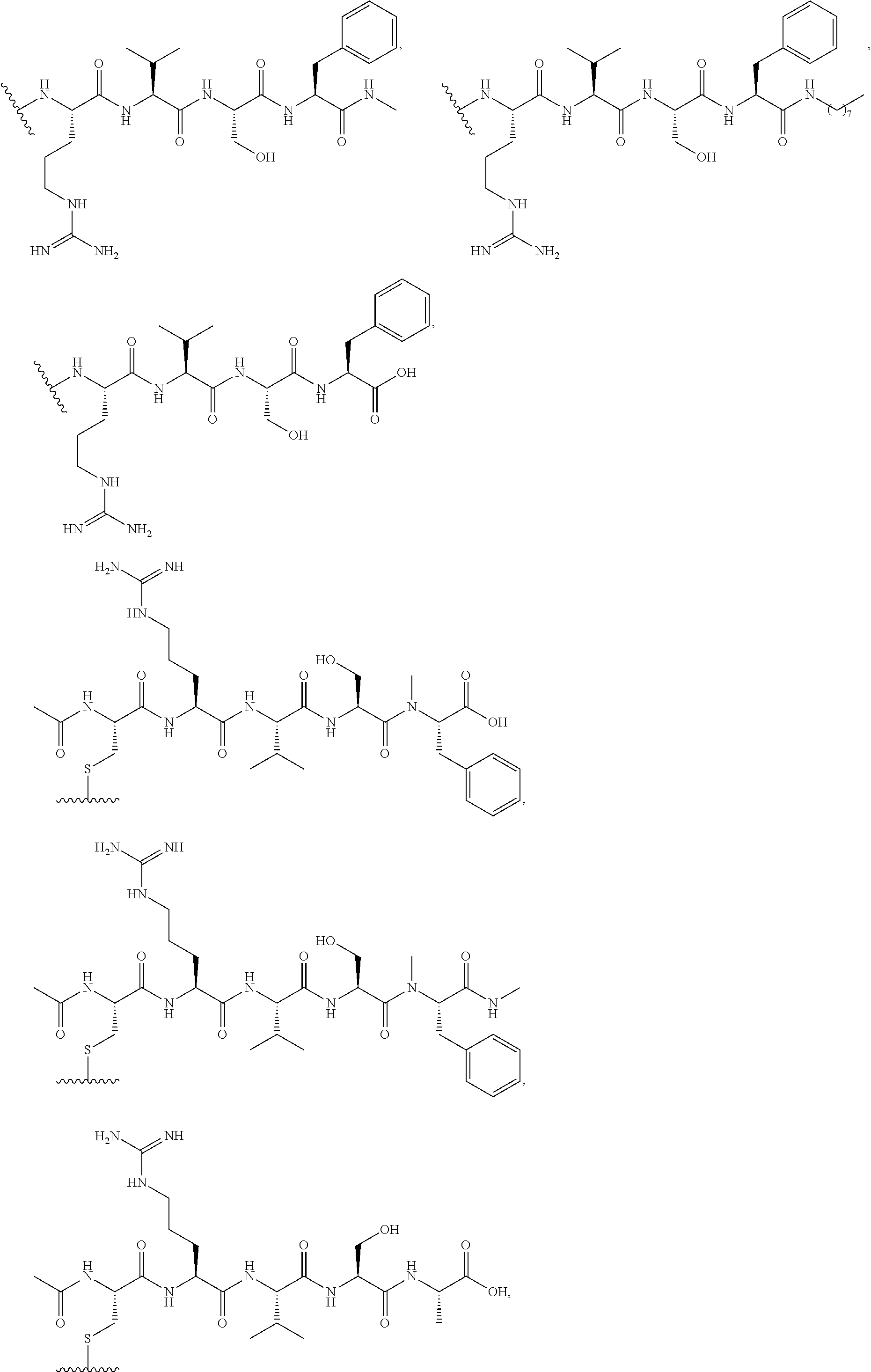

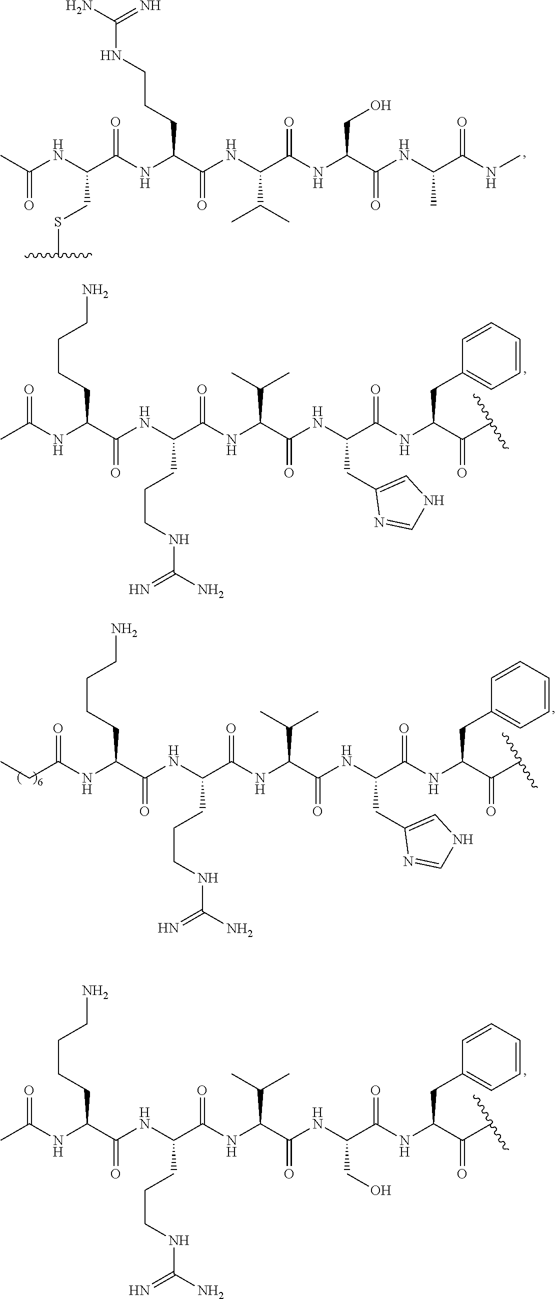

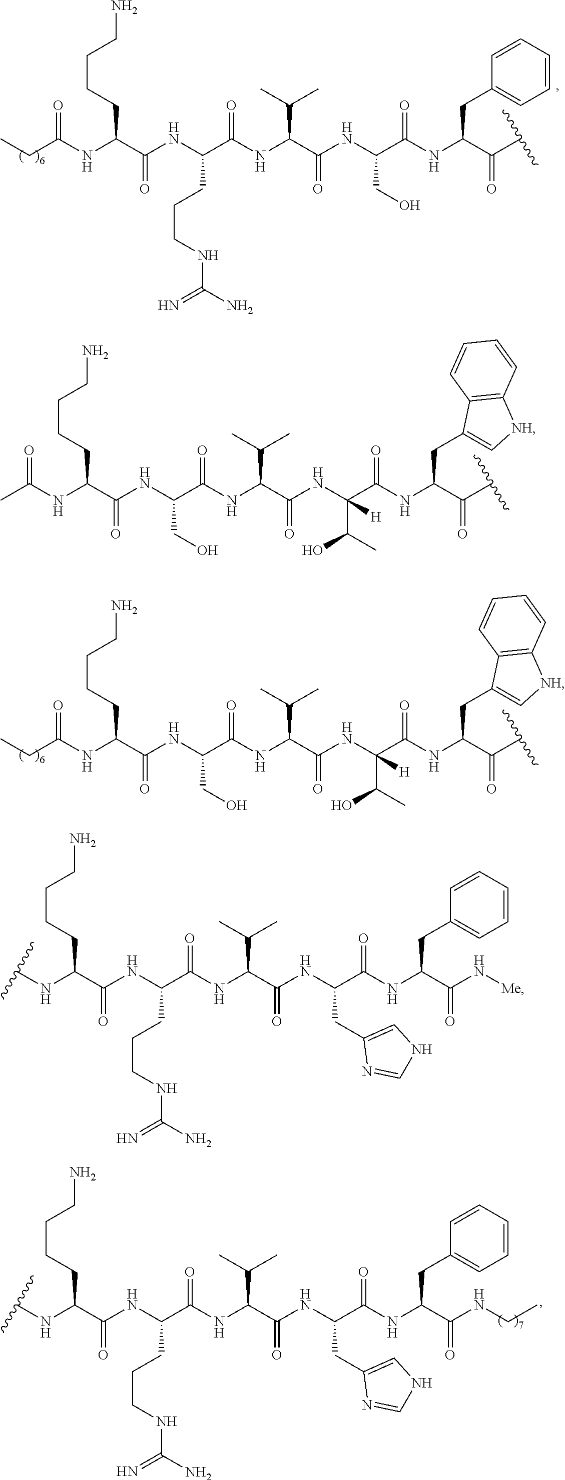

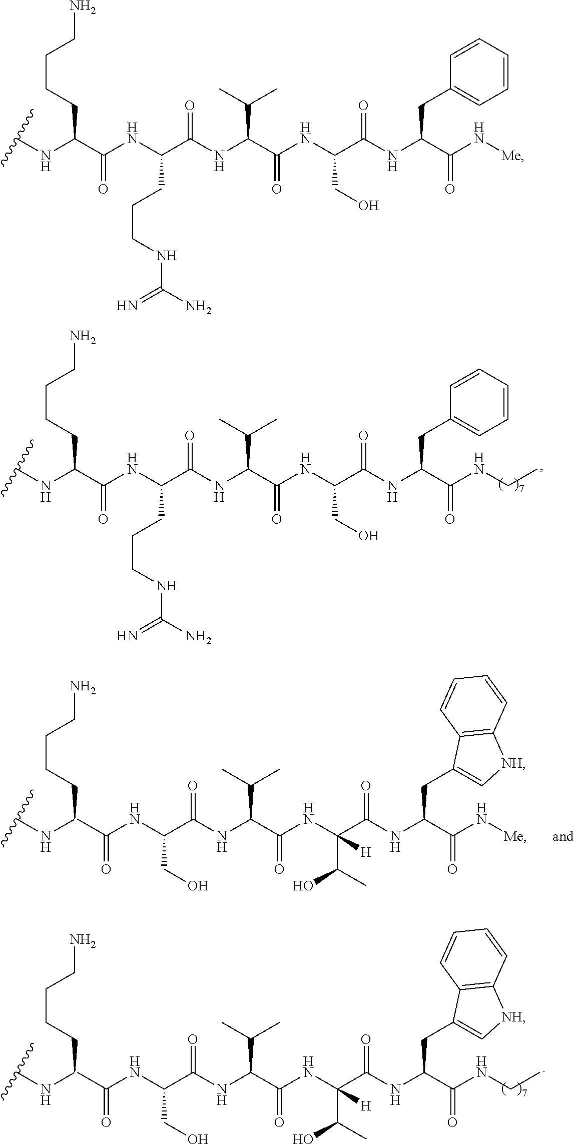

In certain embodiments, the protein phosphatase ligand component of Formula (I) is one

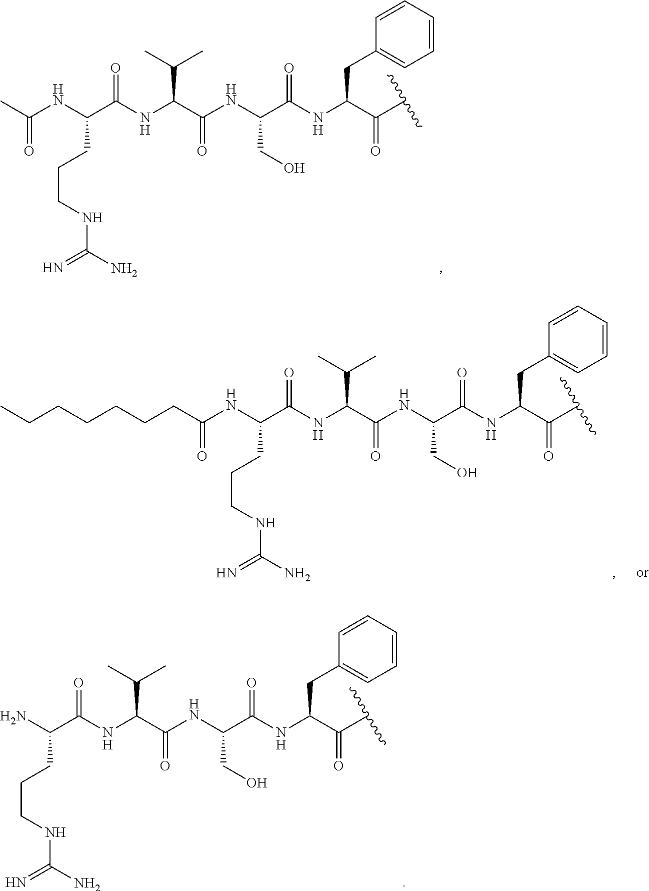 [SMILES: *C(=O)[C@H](Cc1ccccc1)NC(=O)[C@H](CO)NC(=O)[C@@H](NC(=O)[C@H](CCCNC(=N)N)NC(C)=O)C(C)C ; *C(=O)[C@H](Cc1ccccc1)NC(=O)[C@H](CO)NC(=O)[C@@H](NC(=O)[C@H](CCCNC(=N)N)NC(=O)CCCCCCC)C(C)C ; *C(=O)[C@H](Cc1ccccc1)NC(=O)[C@H](CO)NC(=O)[C@@H](NC(=O)[C@@H](N)CCCNC(=N)N)C(C)C]

Embodiments described for the compound of Formula (I) are reiterated for the compound of Formula (I-A).

In certain embodiments, the protein phosphatase ligand is the protein phosphatase ligand component in one of the compounds set forth in Tables 1, 3, 4, 6, 7, 9, 11, 12, 14-16, or 18-20. In certain embodiments, the protein phosphatase ligand is the protein phosphatase ligand component in one of the compounds set forth in Tables 1, 3, 4, 6, 7, 9, 11, 12, 14-16, 18-20, or 22. In certain embodiments, the protein phosphatase ligand is the protein phosphatase ligand component in one of the compounds set forth in Tables 1, 3, 4, 6, 7, 9, 11, 12, 14A, 16A, or 22.

In certain embodiments, the compound is a compound of Formula (I) or a salt thereof.

A non-limiting example of a compound of the invention comprising a protein phosphatase ligand includes, but is not limited to, a compound comprising Formula (II), or a salt, solvate, prodrug, isotopically labelled derivative, stereoisomer (such as, in a non-limiting example, an enantiomer or diastereoisomer, and/or any mixtures thereof, such as, in a non-limiting example, mixtures in any proportions of enantiomers and/or diastereoisomers thereof), tautomer and any mixtures thereof, and/or geometric isomer and any mixtures thereof:

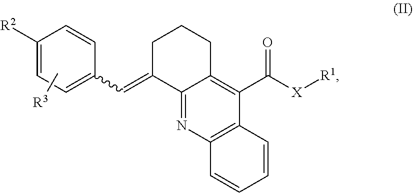

wherein:

X is selected from the group consisting of a bond, —O—, —NH—, and —N(C₁-C₆ alkyl)-;

one selected from the group consisting of R¹, R², and R³ is -LINKER-(target protein ligand); and

the other two are independently selected from the group consisting of optionally substituted C₁-C₆ alkyl, —OH, optionally substituted C₁-C₆ alkoxy, —NH₂, —NH(optionally substituted C₁-C₆ alkyl), and —N(optionally substituted C₁-C₆ alkyl)(optionally substituted C₁-C₆ alkyl).

A non-limiting example of a compound of the invention comprising a protein phosphatase ligand includes, but is not limited to, a compound comprising formula (III), or a salt, solvate, prodrug, isotopically labelled derivative, stereoisomer (such as, in a non-limiting example, an enantiomer or diastereoisomer, and/or any mixtures thereof, such as, in a non-limiting example, mixtures in any proportions of enantiomers and/or diastereoisomers thereof), tautomer and any mixtures thereof, and/or geometric isomer and any mixtures thereof:

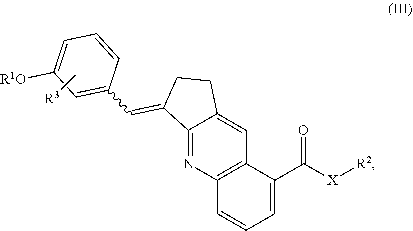

wherein:

X is selected from the group consisting of a bond, —O—, —NH—, and —N(C₁-C₆ alkyl)-;

one of the following applies:

(i) R³ is -LINKER-(target protein ligand), and R¹ and R² are independently selected from the group consisting of H, optionally substituted C₁-C₆ alkyl, and optionally substituted C₃-C₈ cycloalkyl;

(ii) one selected from the group consisting of R¹ and R² is -LINKER-(target protein ligand), and the other is selected from the group consisting of H, optionally substituted C₁-C₆ alkyl, and optionally substituted C₃-C₈ cycloalkyl; and R³ is selected from the group consisting of optionally substituted C₁-C₆ alkyl, —OH, optionally substituted C₁-C₆ alkoxy, —NH₂, —NH(optionally substituted C₁-C₆ alkyl), and —N(optionally substituted C₁-C₆ alkyl)(optionally substituted C₁-C₆ alkyl).

A non-limiting example of a compound of the invention comprising a protein phosphatase ligand includes, but is not limited to, a compound comprising formula (IV), or a salt, solvate, prodrug, isotopically labelled derivative, stereoisomer (such as, in a non-limiting example, an enantiomer or diastereoisomer, and/or any mixtures thereof, such as, in a non-limiting example, mixtures in any proportions of enantiomers and/or diastereoisomers thereof), tautomer and any mixtures thereof, and/or geometric isomer and any mixtures thereof:

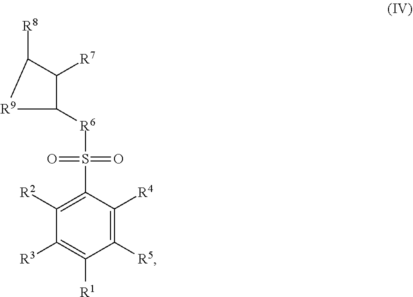

wherein:

R¹ is selected from the group consisting of H, C₁-C₆ haloalkyl, C₁-C₆ haloalkoxy, and -LINKER-(target protein ligand);

each one of R², R³, R⁴, and R⁵ is independently selected from the group consisting of H and -LINKER-(target protein ligand);

R⁶ is selected from the group consisting of —CH₂—, —CH(LINKER-target protein ligand)-, —NH—, and —N(LINKER-target protein ligand)-;

R⁷ is selected from the group consisting of H and OH;

R⁸ is selected from the group consisting of

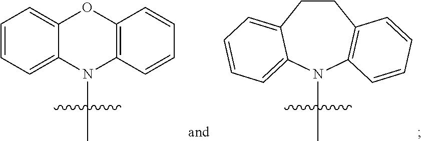 [SMILES: *N1c2ccccc2Oc2ccccc21 ; *N1c2ccccc2CCc2ccccc21]

R⁹ is selected from the group consisting of null (absent), —CH₂—, —CH₂CH₂—, and —CH₂CH₂CH₂—;

with the proviso that only one of R¹-R⁶ comprises -LINKER-(target protein ligand).

A non-limiting example of a compound of the invention comprising a protein phosphatase ligand includes, but is not limited to, a compound comprising formula (V), or a salt, solvate, prodrug, isotopically labelled derivative, stereoisomer (such as, in a non-limiting example, an enantiomer or diastereoisomer, and/or any mixtures thereof, such as, in a non-limiting example, mixtures in any proportions of enantiomers and/or diastereoisomers thereof), tautomer and any mixtures thereof, and/or geometric isomer and any mixtures thereof:

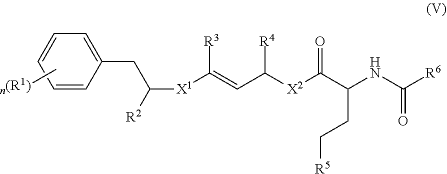

wherein:

each occurrence of R¹, R², R³, R⁴, R⁵, and R⁶ is independently selected from the group consisting of H, -LINKER-(target protein ligand), halogen, NO₂, C₁-C₆ alkyl, C₁-C₆ haloalkyl, C₃-C₈ cycloalkyl, C₂-C₆ alkenyl, C₂-C₆ alkynyl, C₁-C₆ alkoxy, optionally substituted aryl, and optionally substituted heteroaryl, wherein the alkyl, cycloalkyl, alkenyl, or alkynyl is optionally independently substituted with at least one selected from the group consisting of hydroxyl-OR′, NR′R′, amide, —C(═O)OR′, guanidino, —SR′, halogen, C₁-C₆ alkyl, C₃-C₈ cycloalkyl, C₂-C₆ alkenyl, C₁-C₆ alkyl, alkoxy, and heteroaryl, wherein each occurrence of R′ is independently H or C₁-C₆ alkyl;

n is 0, 1, 2, 3, 4, or 5;

X¹ and X² are independently selected from group consisting of —NH—, —O—, C₁-C₆ alkylene, C₃-C₈ cycloalkyene, C₂-C₆ alkenylene, C₂-C₆ alkynylene, C₁-C₆ alkoxydiyl, optionally substituted arylene, and optionally substituted heteroarylene, wherein the alkylene, cycloalkylene, alkenylene, or alkynylene is optionally independently substituted with at least one selected from the group consisting of hydroxyl-OR′, NR′R′, amide, —C(═O)OR′, guanidino, —SR′, halogen, C₁-C₆ alkyl, C₃-C₈ cycloalkyl, C₂-C₆ alkenyl, C₁-C₆ alkyl, alkoxy, and heteroaryl, wherein each occurrence of R′ is independently H or C₁-C₆ alkyl;

with the proviso that only one of R¹-R⁶ comprises -LINKER-(target protein ligand).

In certain embodiments, the compound is a compound in any one of Tables 1, 3, 4, 6, 7, 9, 11, 12, 14-16, or 18-20 herein, or a pharmaceutically acceptable salt thereof. In certain embodiments, the compound is a compound in any one of Tables 1, 3, 4, 6, 7, 9, 11, 12, 14-16, 18-20, or 22 herein, or a pharmaceutically acceptable salt thereof. In certain embodiments, the compound is a compound in any one of Tables 1, 3, 4, 6, 7, 9, 11, 12, 14A, 16A, or 22 herein, or a pharmaceutically acceptable salt thereof. In certain embodiments, the compound is a compound in any one of Tables 24, 25, or 26 herein, or a pharmaceutically acceptable salt thereof.

Target Protein Ligand

In one aspect, the compounds of the invention comprise a ligand of a target protein (“target protein ligand”). Any known target protein ligand is useful within the compositions and methods of the invention.

In certain embodiments, the target protein ligand is a ligand that binds to a target protein listed in Table I-1.

TABLE I-1

| No. | Target Protein |
|---|---|
| 1 | RAS |
| 2 | RAF |
| 3 | MEK (e.g., a MEK1 or MEK2) |
| 4 | ERK |
| 5 | PI3K |
| 6 | AKT (e.g., ATK1, AKT2, or |
| | AKT3) |
| 7 | A-RAF |
| 8 | B-RAF |
| 9 | C-RAF |
| 10 | ERK1 |
| 11 | ERK2 |
| 12 | RSK1 |
| 13 | RSK2 |
| 14 | RSK3 |
| 15 | RSK4 |
| 16 | PIM1 |
| 17 | PKA |
| 18 | PKCI |
| 19 | PKCE |
| 20 | PRKD1 |
| 21 | PKC |
| 22 | p38 |
| 23 | BIM |
| 24 | NOXA |
| 25 | PUMA |
| 26 | BAD |
| 27 | BAK |
| 28 | BOK |
| 29 | TAU |
| 30 | CDK5 |
| 31 | AMPK |
| 32 | GSK3beta |
| 33 | CK1 |
| 34 | MARKs |
| 36 | Dyrk-1A |
| 37 | FYN |
| 38 | ABL |
| 39 | SYK |
| 40 | insulin receptor (IR) |
| 41 | IRS1 |
| 42 | mTOR |
| 43 | FoxO1 |
| 44 | INK |
| 45 | c-JUN |
| 46 | IKKβ |
| 47 | NFkB |
| 48 | SOS1 |
| 49 | Pyruvate Kinase (PKM) |
| 50 | Alpha-synuclein |
| 51 | STAT3 |
| 52 | YAP |
| 53 | EGFR |
| 54 | PDK1 |
| 55 | KRAS |
| 56 | GYS1 |
| 57 | GYS2 |
| 58 | HER2 |
| 59 | Huntingtin |
| 60 | VHL |
| 61 | ITK |
| 62 | FGFR1 |
| 63 | FGFR2 |
| 64 | FGFR3 |
| 65 | FGFR4 |
| 66 | Pyruvate Kinase PKLR |
| 67 | Brd4 |
| 68 | MDM2 |
| 69 | TBK1 |

In certain embodiments, the target protein ligand is a ligand that binds to RAS. In certain embodiments, the target protein ligand is a ligand that binds to RAF. In certain embodiments, the target protein ligand is a ligand that binds to MEK (e.g., a MEK1 or MEK2). In certain embodiments, the target protein ligand is a ligand that binds to ERK. In certain embodiments, the target protein ligand is a ligand that binds to PI3K. In certain embodiments, the target protein ligand is a ligand that binds to AKT (e.g., ATK1, AKT2, or AKT3). In certain embodiments, the target protein ligand is a ligand that binds to A-RAF. In certain embodiments, the target protein ligand is a ligand that binds to B-RAF. In certain embodiments, the target protein ligand is a ligand that binds to C-RAF. In certain embodiments, the target protein ligand is a ligand that binds to ERK1. In certain embodiments, the target protein ligand is a ligand that binds to ERK2. In certain embodiments, the target protein ligand is a ligand that binds to RSK1. In certain embodiments, the target protein ligand is a ligand that binds to RSK2. In certain embodiments, the target protein ligand is a ligand that binds to RSK3. In certain embodiments, the target protein ligand is a ligand that binds to RSK4. In certain embodiments, the target protein ligand is a ligand that binds to PIM1. In certain embodiments, the target protein ligand is a ligand that binds to PKA. In certain embodiments, the target protein ligand is a ligand that binds to PKCI. In certain embodiments, the target protein ligand is a ligand that binds to PKCE. In certain embodiments, the target protein ligand is a ligand that binds to PRKD1. In certain embodiments, the target protein ligand is a ligand that binds to PKC. In certain embodiments, the target protein ligand is a ligand that binds to p38. In certain embodiments, the target protein ligand is a ligand that binds to BIM. In certain embodiments, the target protein ligand is a ligand that binds to NOXA. In certain embodiments, the target protein ligand is a ligand that binds to PUMA. In certain embodiments, the target protein ligand is a ligand that binds to BAD. In certain embodiments, the target protein ligand is a ligand that binds to BAK. In certain embodiments, the target protein ligand is a ligand that binds to BOK. In certain embodiments, the target protein ligand is a ligand that binds to TAU. In certain embodiments, the target protein ligand is a ligand that binds to CDK5. In certain embodiments, the target protein ligand is a ligand that binds to AMPK. In certain embodiments, the target protein ligand is a ligand that binds to GSK3. In certain embodiments, the target protein ligand is a ligand that binds to CK1. In certain embodiments, the target protein ligand is a ligand that binds to MARKs. In certain embodiments, the target protein ligand is a ligand that binds to Dyrk-1A. In certain embodiments, the target protein ligand is a ligand that binds to FYN. In certain embodiments, the target protein ligand is a ligand that binds to ABL. In certain embodiments, the target protein ligand is a ligand that binds to SYK. In certain embodiments, the target protein ligand is a ligand that binds to insulin receptor (IR). In certain embodiments, the target protein ligand is a ligand that binds to IRS1. In certain embodiments, the target protein ligand is a ligand that binds to PI3K. In certain embodiments, the target protein ligand is a ligand that binds to AKT. In certain embodiments, the target protein ligand is a ligand that binds to mTOR. In certain embodiments, the target protein ligand is a ligand that binds to FoxO1. In certain embodiments, the target protein ligand is a ligand that binds to JNK. In certain embodiments, the target protein ligand is a ligand that binds to c-JUN. In certain embodiments, the target protein ligand is a ligand that binds to IKKβ. In certain embodiments, the target protein ligand is a ligand that binds to NFkB. In certain embodiments, the target protein ligand is a ligand that binds to SOS1. In certain embodiments, the target protein ligand is a ligand that binds to Pyruvate Kinase (PKM). In certain embodiments, the target protein ligand is a ligand that binds to Alpha-synuclein. In certain embodiments, the target protein ligand is a ligand that binds to STAT3. In certain embodiments, the target protein ligand is a ligand that binds to YAP. In certain embodiments, the target protein ligand is a ligand that binds to EGFR. In certain embodiments, the target protein ligand is a ligand that binds to PDK1. In certain embodiments, the target protein ligand is a ligand that binds to KRAS. In certain embodiments, the target protein ligand is a ligand that binds to GYS1. In certain embodiments, the target protein ligand is a ligand that binds to GYS2. In certain embodiments, the target protein ligand is a ligand that binds to HER2. In certain embodiments, the target protein ligand is a ligand that binds to Huntingtin. In certain embodiments, the target protein ligand is a ligand that binds to VHL. In certain embodiments, the target protein ligand is a ligand that binds to ITK. In certain embodiments, the target protein ligand is a ligand that binds to FGFR1. In certain embodiments, the target protein ligand is a ligand that binds to FGFR2. In certain embodiments, the target protein ligand is a ligand that binds to FGFR3. In certain embodiments, the target protein ligand is a ligand that binds to FGFR4. In certain embodiments, the target protein ligand is a ligand that binds to Pyruvate Kinase PKLR. In certain embodiments, the target protein ligand is a ligand that binds to Brd4. In certain embodiments, the target protein ligand is a ligand that binds to GSK-3beta. In certain embodiments, the target protein ligand is a ligand that binds to MDM2. In certain embodiments, the target protein ligand is a ligand that binds to TBK1.

In certain embodiments, the target protein is a protein involved in cell proliferation, inflammation, and/or survival, such as but not limited to RAS, RAF, MEK, ERK, PI3K, Akt, A-RAF, B-RAF, C-RAF, ERK1, ERK2, RSK1, RSK2, PIM1, PKA, PKCI, PKCE, PRKD1, PKC, p38, BIM, NOXA, PUMA, BAD, BAK, and/or BOK.

In certain embodiments, the target protein is involved in the Tau aggregation pathway, such as but not limited to TAU, CDK5, AMPK, GSK3, CK1, MARKs, PKA, Dyrk-1A, FYN, ABL, and/or SYK.

In certain embodiments, the target protein is involved in the insulin signaling pathway, such as but not limited to insulin receptor (IR), IRS1, PI3K, AKT, mTOR, FoxO1, GSK3, JNK, c-JUN, IKKβ, and/or NFkB.

In certain embodiments, the target protein ligand is a kinase inhibitor including, but not limited to, Erlotinib, Sunitanib, Sorafenib, Desatinib, Lapatinib, U09-CX-5279, Afatinib, Fostamatinib, Gefitinib, Lenvatinib, Vandetanib, Vemurafenib, Imatinib, Pazopanib, AT-9283, TAE684, Nilotinib, NVP-BSK805, Crizotinib, JNJ FMS, Foretinib, Lestaurtinib, KW-2449, Tamatinib, SU-14813, TG-101348, BIBF-1120, AST-487, PP-242, Bosutinib, JNJ-28312141, Dovitinib, Tozasertib, PD-173955, Crizotinib, PHA-665752, A-674563, and any kinase inhibitors identified in:
Millan, et al., 2011, J. Med. Chem. 54:7797, including the kinase inhibitors Y1W and Y1X;
kinase inhibitors identified in Schenkel, et al., 2011, J. Med. Chem. 54(24):8440-8450, including the compounds 6TP and 0TP;
kinase inhibitors identified in Van Eis, et al., 2011, Biorg. Med. Chem. Lett. 21(24):7367-72, including the kinase inhibitor 07U;
kinase inhibitors identified in Lountos, et al., 2011, J. Struct. Biol. 176:292, including the kinase inhibitors YCF, XK9 and NXP;
inhibitors of BRAF (BRAF^{V600E})/MEK:

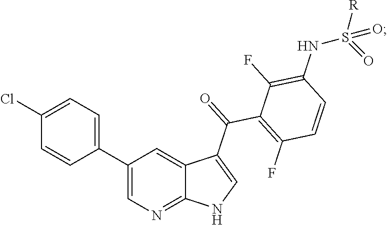

inhibitors of Tyrosine Kinase ABL:

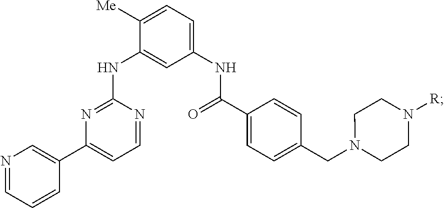

inhibitors of the insulin receptor and the insulin-like growth factor 1 receptor (IGF-1R), including but not limited to:

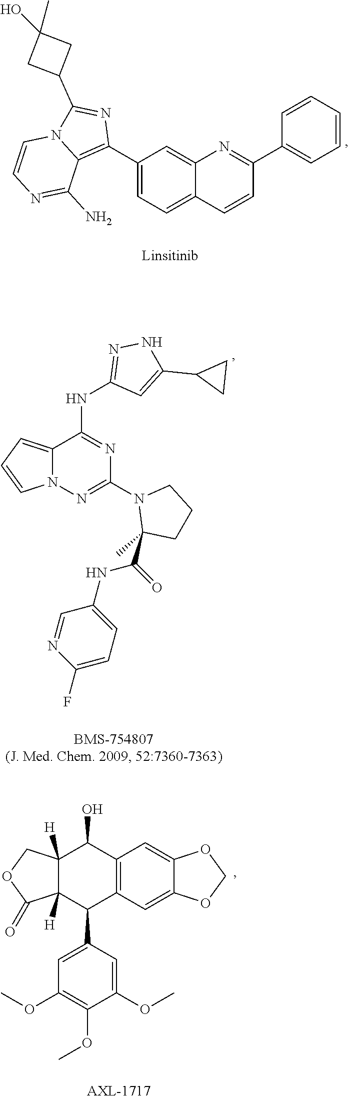 [SMILES: CC1(O)CC(c2nc(-c3ccc4ccc(-c5ccccc5)nc4c3)c3c(N)nccn23)C1 ; C[C@@]1(C(=O)Nc2ccc(F)nc2)CCCN1c1nc(Nc2cc(C3CC3)[nH]n2)c2cccn2n1 ; COc1cc([C@@H]2c3cc4c(cc3[C@H](O)[C@H]3COC(=O)[C@@H]23)OCO4)cc(OC)c1OC]

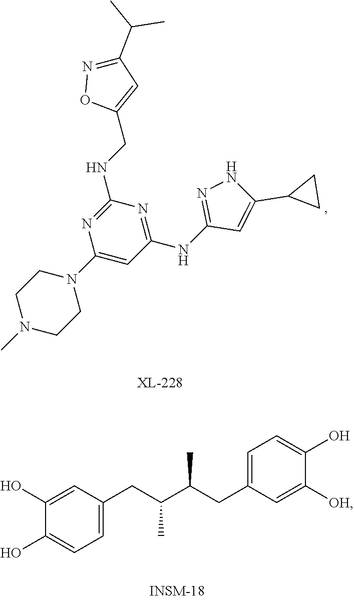 [SMILES: CC(C)c1cc(CNc2nc(Nc3cc(C4CC4)[nH]n3)cc(N3CCN(C)CC3)n2)on1 ; C[C@H](Cc1ccc(O)c(O)c1)[C@@H](C)Cc1ccc(O)c(O)c1]

and any IR/IGF-1R kinase inhibitors identified in:
   Anastassiadis, et al., 2013, J. Biol. Chem. 288(39):28068;
   Miller, et al., 2009, Bioorg. Med. Chem. Lett. 19:62-66:

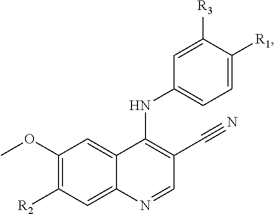

wherein R₁ is 1-Et-4,5-di-Me-2-thio-imidazolyl, 1-Me-2-thio-imidazolyl, 5-di-Me-2-thio-imidazolyl, or 1,4,5-tri-Me-2-thio-imidazolyl; R₂ is —O(CH₂)₃-(4-Et-piperazin-1-yl), —O(CH₂)₃NMe(CH₂)₂OH, —O(CH₂)₃-pyrrolidin-1-yl, —O(CH₂)₃-(4-Et-OH-piperazin-1-yl), —NH(CH₂)₃-(4-Me-piperazin-1-yl), —NH(CH₂)₃NMe₂, —O(CH₂)₃—NHSO₂Me, or —O(CH₂)₃N(Me)CH₂CH(OH)CH₂OH; and R³ is H, Cl, Br, I, or CN.
Mayer, et al., 2008, Bioorg. Med. Chem. Lett. 18:3641-3645:

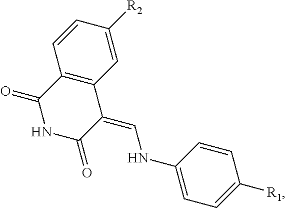

wherein R₁ is H, —CH₂-pyrrolidin-1-yl, —CH₂-piperidin-1-yl, —CH₂NMe₂, —N-Me-piperazin-1-yl, 3,5-di-Me-piperazin-1-yl, or —CH₂NEt₂; R₂ is Br, aniline, o-Cl-aniline, m-Cl-aniline, p-Cl-aniline, m-MeO-aniline, o-Me-aniline, m-Me-aniline, p-Me-aniline, o-CN-aniline, m-CN-aniline, p-CN-aniline, m-Ac-aniline, p-Ac-aniline, m-CF₃-aniline, or Ph-SO₂NH.
Velaparthi, et al., 2007, Bioorg. Med. Chem. Lett. 17:2317-2321:

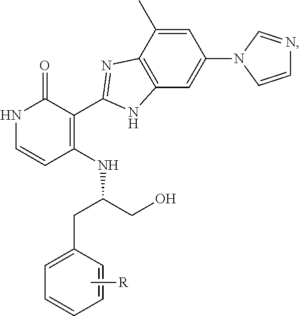

wherein R is H, Br, Cl, F, cyano, methoxy, tert-butoxy, trifluoromethyl, nitro, trifluoromethoxy, or methyl.
Sampognaro, et al., 2010, Bioorg. Med. (hem. Lett. 20:5027-5030:

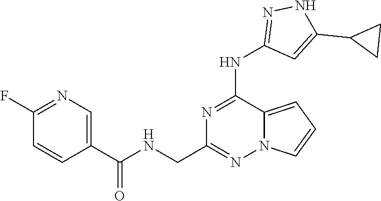 [SMILES: O=C(NCc1nc(Nc2cc(C3CC3)[nH]n2)c2cccn2n1)c1ccc(F)nc1]

Chamberlain, et al., 2009, Bioorg. Med. (hem. Lett. 19:469-473:

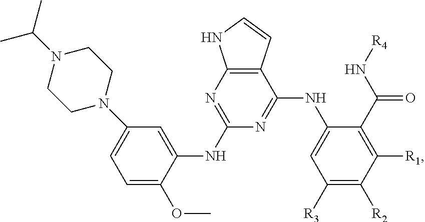

wherein R₁ is H, Cl, or F; R₂ is H, Cl, or F; R₃ is H, Cl, or F; R₄ is H, Me, OH, 2-hydroxyethyl, 2-methoxyethyl, or 2,3-hydroxypropyl.
Nemecek, et al., 2010, Chem. Biol. Drug. Des. 76:100-106:

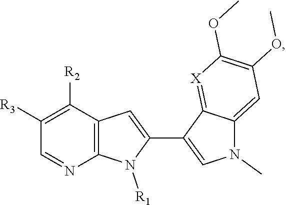

wherein X is C or N; R₁ is methyl, —CH₂CH₂(morpholin-4-yl), —CH₂CH₂(N⁴-methyl-piperazin-1-yl); R₂ is H or Cl; R₃ is H or F.
Lesuisse, et al., 2011, Bioorg. Med. Chem. Lett. 21:2224-2228:

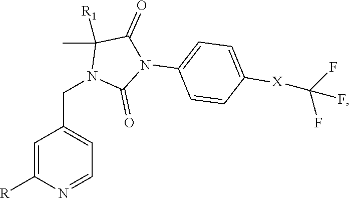

wherein X is S or SO₂; R is H, ethyl, F, Cl, CN, —NH₂, piperidin-1-yl, morpholin-4-yl, piperazin-1-yl, —C(═O)NHMe, —NHMe, -NHEt, —NHAc, or NHiPr; and R₁ is H, CF₃, or Me.
Heinrich, et al., 2010, ACS Med. Chem. Lett. 1:199-203:

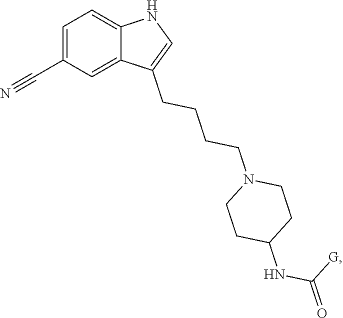

wherein G is

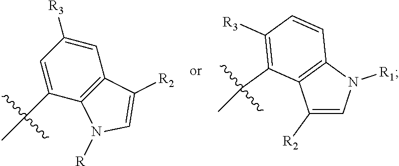

R₁ is H, methyl, or ethyl; R₂ is H, cyano, —CHO, —Ac, —COCF₃, or —C(═O)NH₂; R₃ is H or F.

In certain embodiments, the target protein ligand has the following formula:

-(optionally substituted 3-10 membered heteroalkylene)-(optionally substituted C₁-C₁₀ alkylene)-Cl.

In certain embodiments, the target protein ligand has the following formula:

-(3-10 membered heteroalkylene)-(C₁-C₁₀ alkylene)-Cl,

wherein each of the 3-10 membered heteroalkylene and C₁-C₁₀ alkylene is optionally substituted with 1, 2, or 3 substituents independently selected from halogen, C₁-C₆ alkyl, C₁-C₆ haloalkyl, C₃-C₆ cycloalkyl, hydroxyl, C₁-C₆ alkoxy, and cyano.

In certain embodiments, the target protein ligand has the following formula:

-(3-10 membered heteroalkylene)-(C₁-C₁₀ alkylene)-Cl

In certain embodiments, the target protein ligand has the following formula:

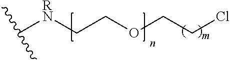

wherein:
R is hydrogen or C₁-C₆ alkyl;
m is 1, 2, 3, 4, 5, 6, 7, 8, 9, or 10; and
n is 0, 1, 2, 3, or 4.

In certain embodiments, the target protein ligand has the following formula:

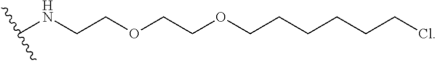 [SMILES: *NCCOCCOCCCCCCCl]

In certain embodiments, the target protein ligand has the following formula:

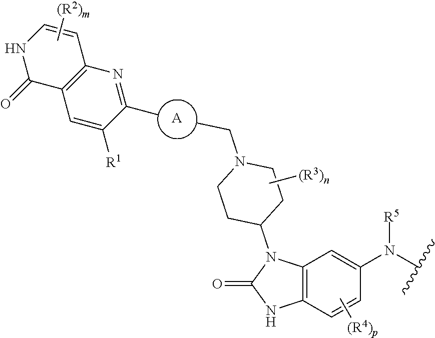

wherein:
A is an optionally substituted phenylene or an optionally substituted 5-6 membered heteroarylene;
R¹ is aryl, heteroaryl, or C₃-C₅₈ cycloalkyl, each of which is optionally substituted;
R², R³, and R⁴ each represent independently for each occurrence halogen, C₁-C₆ alkyl, C₁-C₆ haloalkyl, C₃-C₆ cycloalkyl, hydroxyl, C₁-C₆ alkoxy, or cyano;
R⁵ is hydrogen or optionally substituted C₁-C₆ alkyl; and
m, n, and p are independently 0, 1, or 2.

In certain embodiments, the target protein ligand has the following formula:

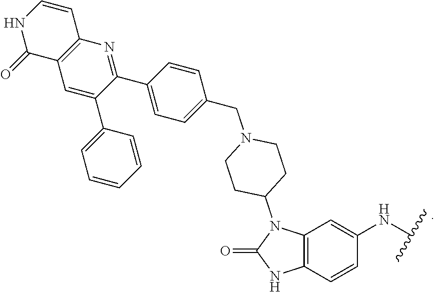 [SMILES: *Nc1ccc2[nH]c(=O)n(C3CCN(Cc4ccc(-c5nc6cc[nH]c(=O)c6cc5-c5ccccc5)cc4)CC3)c2c1]

In certain embodiments, the target protein ligand has the following formula:

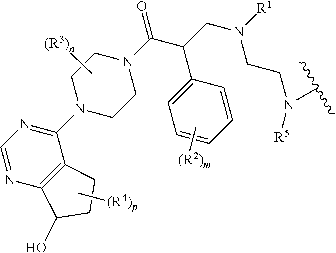

wherein:
R¹ and R⁵ are independently hydrogen or optionally substituted C₁-C₆ alkyl;
R², R³, and R⁴ each represent independently for each occurrence halogen, C₁-C₆ alkyl, C₁-C₆ haloalkyl, C₃-C₆ cycloalkyl, hydroxyl, C₁-C₆ alkoxy, or cyano; and
m, n, and p are independently 0, 1, 2, or 3.

In certain embodiments, the target protein ligand has the following formula:

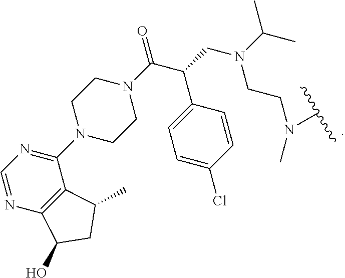 [SMILES: *N(C)CCN(C[C@@H](C(=O)N1CCN(c2ncnc3c2[C@H](C)C[C@H]3O)CC1)c1ccc(Cl)cc1)C(C)C]

In certain embodiments, the target protein ligand has the following formula:

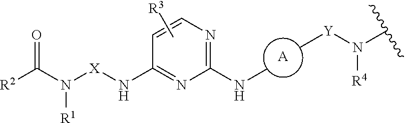

wherein:
A is an optionally substituted phenylene or an optionally substituted 5-6 membered heteroarylene;
R¹ and R⁴ are independently hydrogen or optionally substituted C₁-C₆ alkyl;
R² is C₃-C₈ cycloalkyl, phenyl, or 5-6 membered heteroaryl, each of which is optionally substituted;
R³ is halogen, C₁-C₆ alkyl, C₁-C₆ haloalkyl, C₃-C₆ cycloalkyl, hydroxyl, C₁-C₆ alkoxy, or cyano;
X is optionally substituted C₂-C₆ alkylene; and
Y is optionally substituted 3-6 membered heteroalkylene.

In certain embodiments, the target protein ligand component of Formula (I) is one of the following

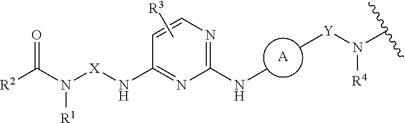

wherein:

A is phenylene;

R¹ and R⁴ are independently hydrogen or C₁-C₆ alkyl;

R² is C₃-C₈ cycloalkyl;

R³ is halogen, C₁-C₆ alkyl, C₁-C₆ haloalkyl, or cyano;

X is C₂-C₆ alkylene; and

Y is 3-6 membered heteroalkylene.

In certain embodiments, the target protein ligand has the following formula:

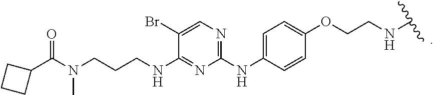 [SMILES: *NCCOc1ccc(Nc2ncc(Br)c(NCCCN(C)C(=O)C3CCC3)n2)cc1]

In certain embodiments, the target protein ligand has the following formula:

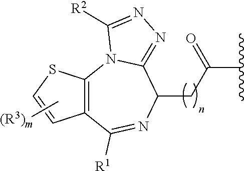

wherein:
R¹ is phenyl, 5-6 membered heteroaryl, or C₃-C₈ cycloalkyl, each of which is optionally substituted;
R² and each R³ represent independently for each occurrence halogen, C₁-C₆ alkyl, C₁-C₆ haloalkyl, C₃-C₆ cycloalkyl, hydroxyl, C₁-C₆ alkoxy, or cyano;
m is 0, 1, or 2; and
n is 0, 1, 2, 3, or 4.

In certain embodiments, the target protein ligand has the following formula:

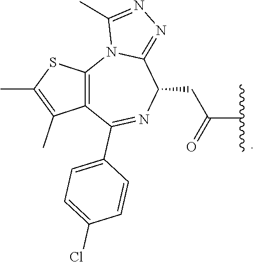 [SMILES: *C(=O)C[C@@H]1N=C(c2ccc(Cl)cc2)c2c(sc(C)c2C)-n2c(C)nnc21]

In certain embodiments, the target protein ligand is one of the following:

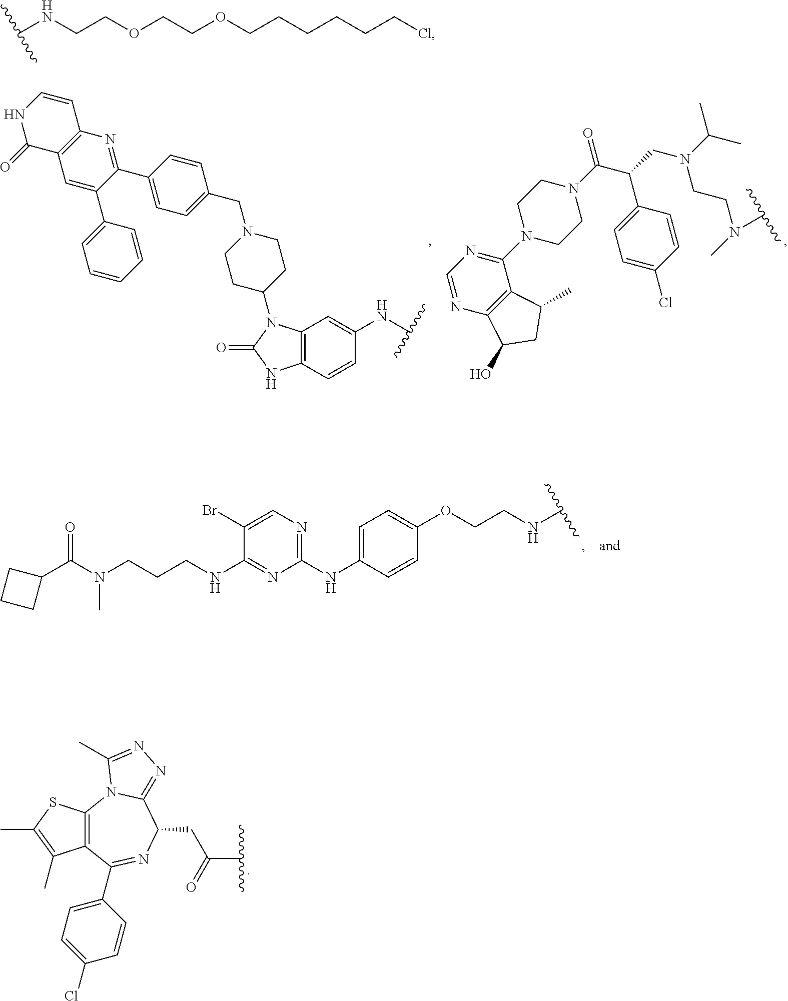 [SMILES: *NCCOCCOCCCCCCCl ; *Nc1ccc2[nH]c(=O)n(C3CCN(Cc4ccc(-c5nc6cc[nH]c(=O)c6cc5-c5ccccc5)cc4)CC3)c2c1 ; *N(C)CCN(C[C@@H](C(=O)N1CCN(c2ncnc3c2[C@H](C)C[C@H]3O)CC1)c1ccc(Cl)cc1)C(C)C ; *NCCOc1ccc(Nc2ncc(Br)c(NCCCN(C)C(=O)C3CCC3)n2)cc1 ; *C(=O)C[C@@H]1N=C(c2ccc(Cl)cc2)c2c(sc(C)c2C)-n2c(C)nnc21]

In certain embodiments, the target protein ligand is an inhibitor and/or binder of Son of Sevenless Homolog 1 (SOS-1). Inhibitors and/or binders of SOS-1 are reported in the literature. Exemplary inhibitors and/or binders of SOS-1 include the following compounds:

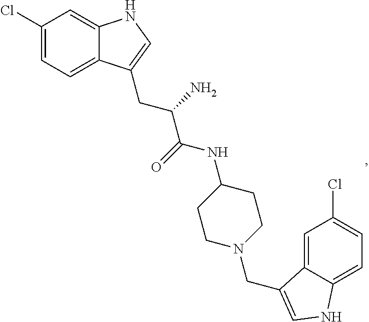 [SMILES: N[C@@H](Cc1c[nH]c2cc(Cl)ccc12)C(=O)NC1CCN(Cc2c[nH]c3ccc(Cl)cc23)CC1]

as described in Akan, D. T. et al., ACS Chem. Biol. 2019, vol. 14(3), page 325;

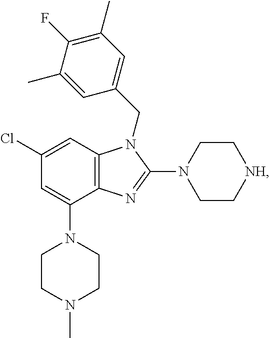 [SMILES: Cc1cc(Cn2c(N3CCNCC3)nc3c(N4CCN(C)CC4)cc(Cl)cc32)cc(C)c1F]

as described in Hodges, T. R. et al., J. Med. Chem. 2018, vol. 61(19), page 8875;

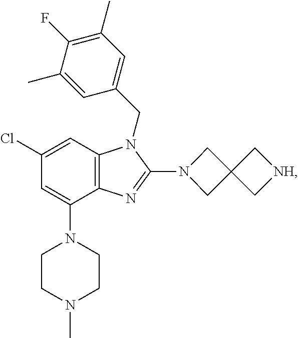 [SMILES: Cc1cc(Cn2c(N3CC4(CNC4)C3)nc3c(N4CCN(C)CC4)cc(Cl)cc32)cc(C)c1F]

as described in Akan, D. T. et al., ACS Chem. Biol. 2019, vol. 14(3), page 325;

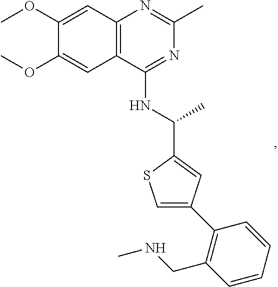 [SMILES: CNCc1ccccc1-c1csc([C@@H](C)Nc2nc(C)nc3cc(OC)c(OC)cc23)c1]

as described in Hillig, R. C. et al., Proc. Nat. Acad. Sci., 2019, vol. 116(7), page 2551;

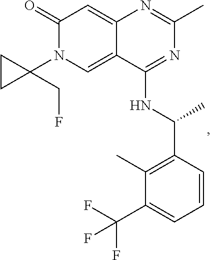 [SMILES: Cc1nc(N[C@H](C)c2cccc(C(F)(F)F)c2C)c2cn(C3(CF)CC3)c(=O)cc2n1]

as described in Ramharter, J. et al., WO 2019/122129;

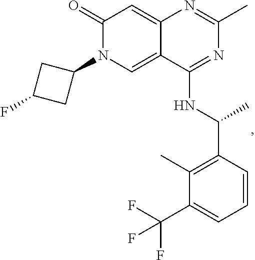 [SMILES: Cc1nc(N[C@H](C)c2cccc(C(F)(F)F)c2C)c2cn([C@H]3C[C@H](F)C3)c(=O)cc2n1]

as described in Ramharter, J. et al, WO 2019/122129;

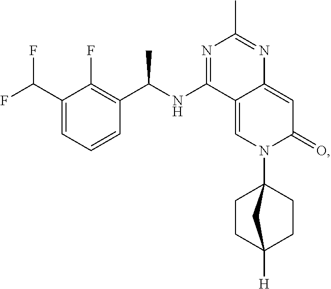 [SMILES: Cc1nc(N[C@H](C)c2cccc(C(F)F)c2F)c2cn([C@]34CC[C@H](CC3)C4)c(=O)cc2n1]

as described in Ramharter, J. et al, WO 2019/122129.

In certain embodiments, the target protein ligand is an inhibitor of Yes-associated protein 1 (YAP1). Inhibitors of YAP1 are reported in the literature. Exemplary inhibitors of YAP1 include the following compounds:

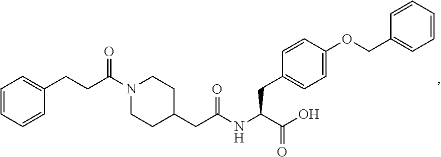 [SMILES: O=C(CC1CCN(C(=O)CCc2ccccc2)CC1)N[C@@H](Cc1ccc(OCc2ccccc2)cc1)C(=O)O]

as described in Chellapan, S. et al., WO 2018/053446;

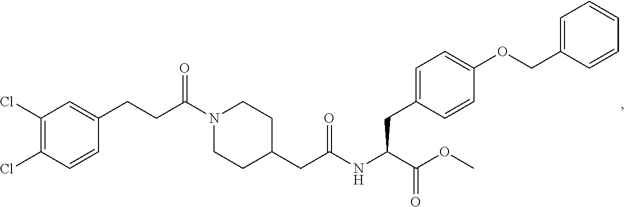 [SMILES: COC(=O)[C@H](Cc1ccc(OCc2ccccc2)cc1)NC(=O)CC1CCN(C(=O)CCc2ccc(Cl)c(Cl)c2)CC1]

as described in Chellapan, S. et al., WO 2018/053446;

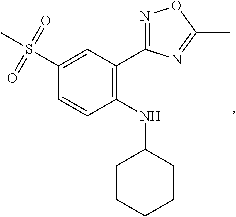

as described in Konradi, A. W. et al., WO 2019/040380;

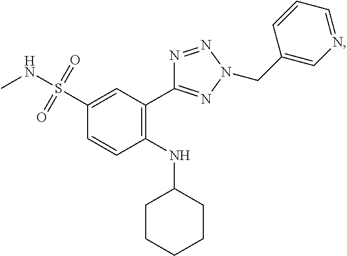 [SMILES: CNS(=O)(=O)c1ccc(NC2CCCCC2)c(-c2nnn(Cc3cccnc3)n2)c1]

as described in Konradi, A. W. et al., WO 2019/040380;

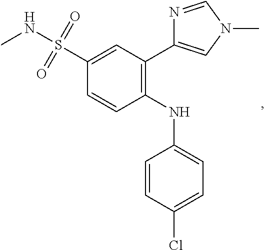

as described in Konradi, A. W. et al., WO 2019/040380;

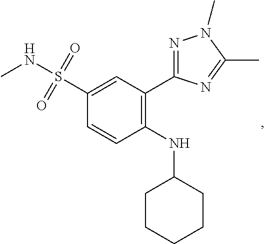

as described in Konradi, A. W. et al., WO 2019/040380;

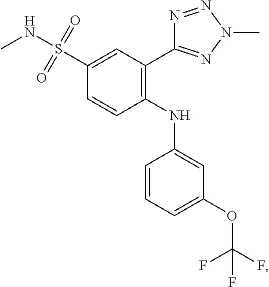 [SMILES: CNS(=O)(=O)c1ccc(Nc2cccc(OC(F)(F)F)c2)c(-c2nnn(C)n2)c1]

as described in Konradi, A. W. et al., WO 2019/040380;

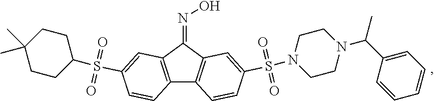 [SMILES: CC(c1ccccc1)N1CCN(S(=O)(=O)c2ccc3c(c2)/C(=N\O)c2cc(S(=O)(=O)C4CCC(C)(C)CC4)ccc2-3)CC1]

as described in Lin, T. T.-L. T. et al., WO 2017/058716;

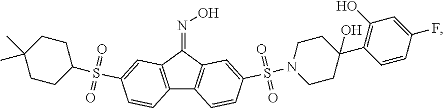 [SMILES: CC1(C)CCC(S(=O)(=O)c2ccc3c(c2)/C(=N/O)c2cc(S(=O)(=O)N4CCC(O)(c5ccc(F)cc5O)CC4)ccc2-3)CC1]

as described in Lin, T. T.-L. T. et al., WO 2017/058716;

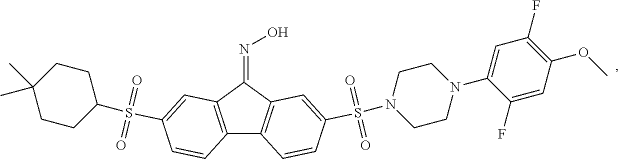

as described in Lin, T. T.-L. T. et al., WO 2017/058716;

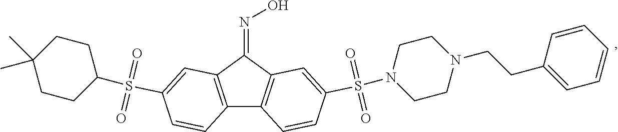 [SMILES: CC1(C)CCC(S(=O)(=O)c2ccc3c(c2)/C(=N/O)c2cc(S(=O)(=O)N4CCN(CCc5ccccc5)CC4)ccc2-3)CC1]

as described in Lin, T. T.-L. T. et al., WO 2017/058716;

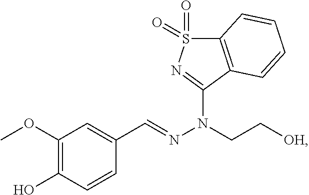 [SMILES: COc1cc(/C=N/N(CCO)C2=NS(=O)(=O)c3ccccc32)ccc1O]

as described in Barth, M. et al., WO 2017/064277;

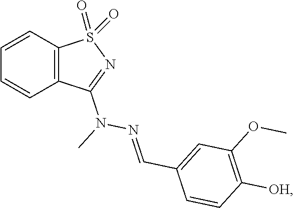 [SMILES: COc1cc(/C=N/N(C)C2=NS(=O)(=O)c3ccccc32)ccc1O]

as described in Barth, M. et al., WO 2017/064277.

In certain embodiments, the target protein ligand is an inhibitor of ribosomal protein S6 kinase alpha-1 (RSK1). Inhibitors of RSK1 are reported in the literature. Exemplary inhibitors of RSK1 include the following compounds:

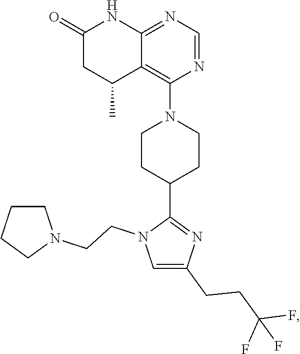 [SMILES: C[C@@H]1CC(=O)Nc2ncnc(N3CCC(c4nc(CCC(F)(F)F)cn4CCN4CCCC4)CC3)c21]

as described in Parthasarathy, S. et al., Bioorg. Med. Chem. Lett. 2018, vol. 28(10), page 1887;

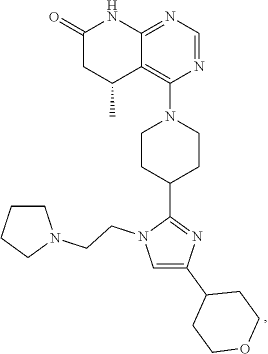 [SMILES: C[C@@H]1CC(=O)Nc2ncnc(N3CCC(c4nc(C5CCOCC5)cn4CCN4CCCC4)CC3)c21]

as described in Parthasarathy, S. et al., Bioorg. Med. Chem. Lett. 2018, vol. 28(10), page 1887;

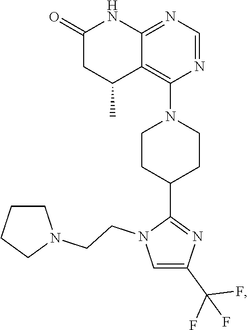 [SMILES: C[C@@H]1CC(=O)Nc2ncnc(N3CCC(c4nc(C(F)(F)F)cn4CCN4CCCC4)CC3)c21]

as described in Parthasarathy, S. et al., Bioorg. Med. Chem. Lett. 2018, vol. 28(10), page 1887;

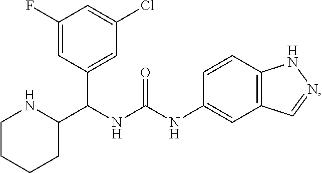 [SMILES: O=C(Nc1ccc2[nH]ncc2c1)NC(c1cc(F)cc(Cl)c1)C1CCCCN1]

as described in Aletru, M. et al., WO 2009/010660;

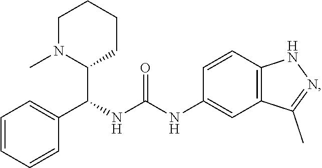 [SMILES: Cc1n[nH]c2ccc(NC(=O)N[C@H](c3ccccc3)[C@H]3CCCCN3C)cc12]

as described in Aletru, M. et al., WO 2009/010660;

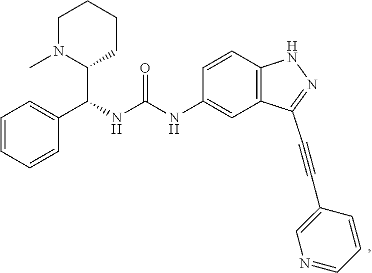 [SMILES: CN1CCCC[C@@H]1[C@H](NC(=O)Nc1ccc2[nH]nc(C#Cc3cccnc3)c2c1)c1ccccc1]

as described in Aletru, M. et al., WO 2009/010660;

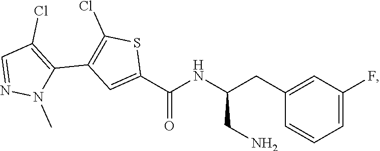 [SMILES: Cn1ncc(Cl)c1-c1cc(C(=O)N[C@H](CN)Cc2cccc(F)c2)sc1Cl]

as described in Dumble, M. et al., PLoS ONE 2014, vol. 9(6), page e100880;

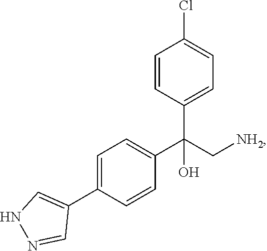 [SMILES: NCC(O)(c1ccc(Cl)cc1)c1ccc(-c2cn[nH]c2)cc1]

as described in Yap, T. A. et al., Clin Cancer Res 2012, vol. 18(14), page 3912;

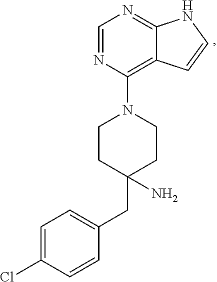 [SMILES: NC1(Cc2ccc(Cl)cc2)CCN(c2ncnc3[nH]ccc23)CC1]

as described in Yap, T. A. et al., Mol. Cancer Ther. 2011, vol. 10(2), page 360.

In certain embodiments, the target protein ligand binds to Bcl2-Associated Agonist of Cell Death (BAD). Binders of BAD are reported in the literature. An exemplary binder of BAD is the following compound:

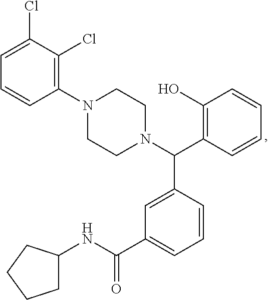 [SMILES: O=C(NC1CCCC1)c1cccc(C(c2ccccc2O)N2CCN(c3cccc(Cl)c3Cl)CC2)c1]

as described in Lobie, P. E. et al., WO 2018/194520.

In certain embodiments, the target protein ligand inhibits and/or binds to Insulin Receptor Substrate 1 (IRS-1). IRS-1 inhibitors and/or binders are reported in the literature. Exemplary inhibitors and/or binders of IRS1 include the following compounds:

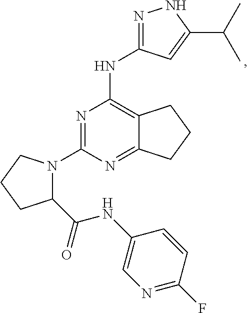 [SMILES: CC(C)c1cc(Nc2nc(N3CCCC3C(=O)Nc3ccc(F)nc3)nc3c2CCC3)n[nH]1]

as described in Chakravarty, S. et al., WO 2014/105958.

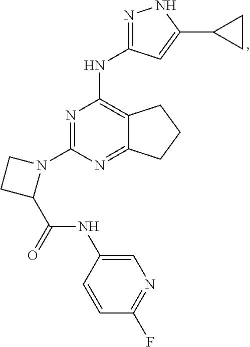 [SMILES: O=C(Nc1ccc(F)nc1)C1CCN1c1nc2c(c(Nc3cc(C4CC4)[nH]n3)n1)CCC2]

as described in Chakravarty, S. et al., WO 2014/105958.

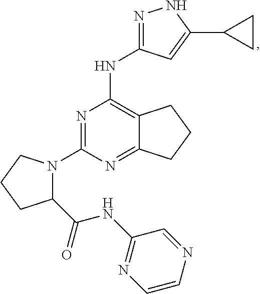 [SMILES: O=C(Nc1cnccn1)C1CCCN1c1nc2c(c(Nc3cc(C4CC4)[nH]n3)n1)CCC2]

as described in Chakravarty, S. et al., WO 2014/105958.

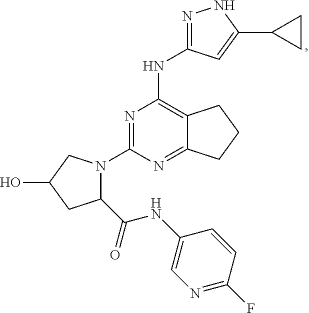 [SMILES: O=C(Nc1ccc(F)nc1)C1CC(O)CN1c1nc2c(c(Nc3cc(C4CC4)[nH]n3)n1)CCC2]

as described in Chakravarty, S. et al., WO 2014/105958.

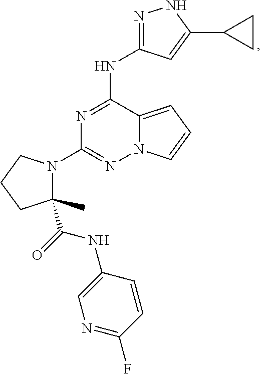 [SMILES: C[C@@]1(C(=O)Nc2ccc(F)nc2)CCCN1c1nc(Nc2cc(C3CC3)[nH]n2)c2cccn2n1]

as described in Chakravarty, S. et al., WO 2014/105958.

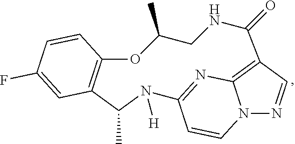 [SMILES: C[C@H]1CNC(=O)c2cnn3ccc(nc23)N[C@H](C)c2cc(F)ccc2O1]

as described in Cui, J. J. et al., WO 2017/015367.

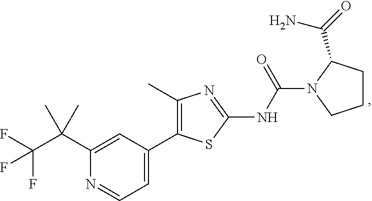 [SMILES: Cc1nc(NC(=O)N2CCC[C@H]2C(N)=O)sc1-c1ccnc(C(C)(C)C(F)(F)F)c1]

as described in Fritsch, C. et al., Mol. Cancer Ther. 2014, vol. 13(5), page 1117.

In certain embodiments, the target protein ligand binds to mutated Kirsten rat sarcoma 2 viral oncogene homolog (K-Ras). Binders of K-Ras are reported in the literature. Exemplary binders of K-Ras include the following compounds:

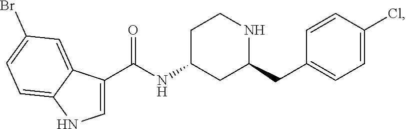 [SMILES: O=C(N[C@@H]1CCN[C@@H](Cc2ccc(Cl)cc2)C1)c1c[nH]c2ccc(Br)cc12]

as described in Jansen, J. M. et al., 24th Int Symp Med Chem (August 28-September 1, Manchester) 2016, Abstract LE007;

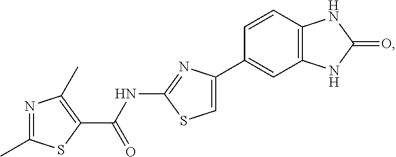 [SMILES: Cc1nc(C)c(C(=O)Nc2nc(-c3ccc4[nH]c(=O)[nH]c4c3)cs2)s1]

as described in Rabizadeh, S. et al., WO 2016/161361;

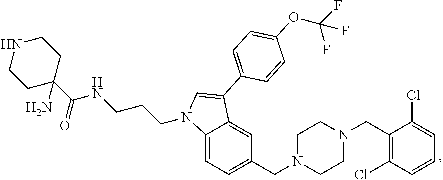 [SMILES: NC1(C(=O)NCCCn2cc(-c3ccc(OC(F)(F)F)cc3)c3cc(CN4CCN(Cc5c(Cl)cccc5Cl)CC4)ccc32)CCNCC1]

as described in Welsch, M. E. et al., Cell 2017, vol. 168(5), page 878;

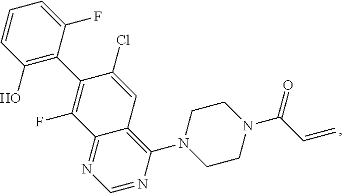 [SMILES: C=CC(=O)N1CCN(c2ncnc3c(F)c(-c4c(O)cccc4F)c(Cl)cc23)CC1]

as described in Wijeratne, A. et al., ACS Med Chem Lett 2018, vol. 9(6), page 557.

In certain embodiments, the target protein ligand binds to Ribosomal protein S6 kinase alpha-6 (RSK4). Binders of RSK4 are reported in the literature. Exemplary binders of RSK4 include the following compounds:

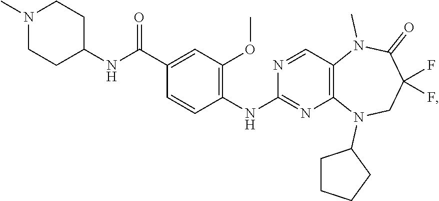 [SMILES: COc1cc(C(=O)NC2CCN(C)CC2)ccc1Nc1ncc2c(n1)N(C1CCCC1)CC(F)(F)C(=O)N2C]

as described in Cheung, A. K. L. et al., Mol Cancer Ther 2013, vol. 12(8), page 1393;

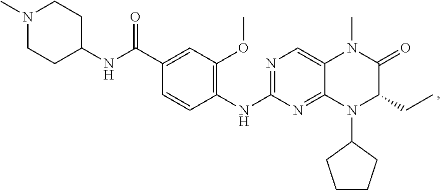 [SMILES: CC[C@H]1C(=O)N(C)c2cnc(Nc3ccc(C(=O)NC4CCN(C)CC4)cc3OC)nc2N1C1CCCC1]

as described in Ciceri, P. et al., Nat Chem Biol 2014, vol. 10(4), page 305;

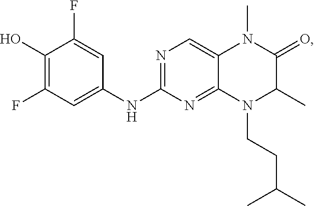 [SMILES: CC(C)CCN1c2nc(Nc3cc(F)c(O)c(F)c3)ncc2N(C)C(=O)C1C]

as described in Ciceri, P. et al., Nat Chem Biol 2014, vol. 10(4), page 305;

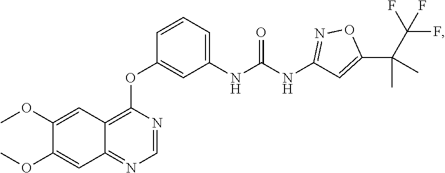 [SMILES: COc1cc2ncnc(Oc3cccc(NC(=O)Nc4cc(C(C)(C)C(F)(F)F)on4)c3)c2cc1OC]

as described in James, J. et al., Mol Cancer Ther 2012, vol. 11(4), page 930;

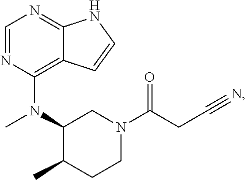 [SMILES: C[C@@H]1CCN(C(=O)CC#N)C[C@@H]1N(C)c1ncnc2[nH]ccc12]

as described in Jiang, J. K. et al., J Med Chem 2008, vol. 51(24), page 8012;

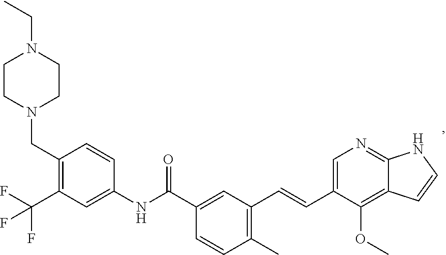 [SMILES: CCN1CCN(Cc2ccc(NC(=O)c3ccc(C)c(/C=C/c4cnc5[nH]ccc5c4OC)c3)cc2C(F)(F)F)CC1]

as described in Tan, L. et al., J Med Chem 2015, vol. 58(1), page 183;

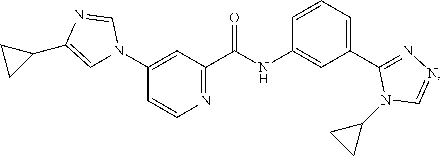 [SMILES: O=C(Nc1cccc(-c2nncn2C2CC2)c1)c1cc(-n2cnc(C3CC3)c2)ccn1]

as described in Liles, J. T. et al., J Med Chem 2008, vol. 51(24), page 8012;

In certain embodiments, the target protein ligand inhibits Ribosomal protein S6 kinase alpha-6 (RSK4). Inhibitors of RSK4 are reported in the literature. Exemplary inhibitors of RSK4 include the following compounds:

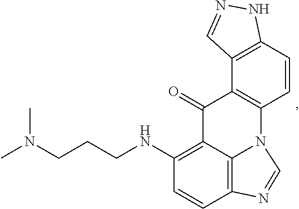 [SMILES: CN(C)CCCNc1ccc2ncn3c4ccc5[nH]ncc5c4c(=O)c1c23]

as described in Ajami, A. M. et al., WO 2009/017795;

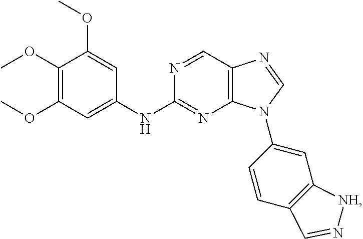 [SMILES: COc1cc(Nc2ncc3ncn(-c4ccc5cn[nH]c5c4)c3n2)cc(OC)c1OC]

as described in Ajami, A. M. et al., WO 2011/106168;

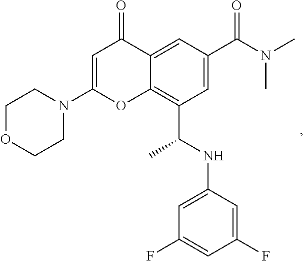 [SMILES: C[C@@H](Nc1cc(F)cc(F)c1)c1cc(C(=O)N(C)C)cc2c(=O)cc(N3CCOCC3)oc12]

as described in Barlaam, B. et al., J Med Chem 2015, vol 58(2), page 943;

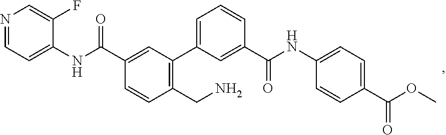 [SMILES: COC(=O)c1ccc(NC(=O)c2cccc(-c3cc(C(=O)Nc4ccncc4F)ccc3CN)c2)cc1]

as described in Boland, S. et al., J Med Chem 2015, vol 58(10), page 4309;

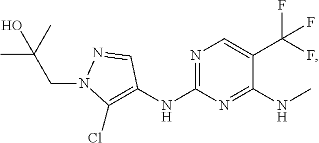 [SMILES: CNc1nc(Nc2cnn(CC(C)(C)O)c2Cl)ncc1C(F)(F)F]

as described in Chan, B. K. et al., ACS Med Chem Lett 2013, vol. 4(1), page 85;

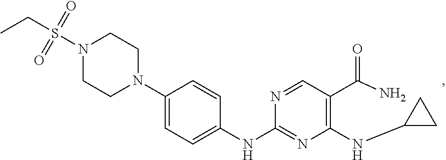 [SMILES: CCS(=O)(=O)N1CCN(c2ccc(Nc3ncc(C(N)=O)c(NC4CC4)n3)cc2)CC1]

as described in Coffey, G. et al., J Pharmacol Exp Ther 2014, vol. 351(3), page 538;

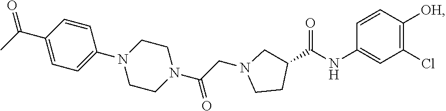 [SMILES: CC(=O)c1ccc(N2CCN(C(=O)CN3CC[C@@H](C(=O)Nc4ccc(O)c(Cl)c4)C3)CC2)cc1]

as described in Deng, Y. et al., J Med Chem 2014, vol 57(21, page 8817;

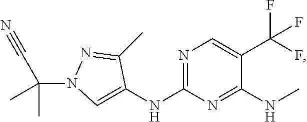 [SMILES: CNc1nc(Nc2cn(C(C)(C)C#N)nc2C)ncc1C(F)(F)F]

as described in Estrada, A. A. et al., J Med Chem 2014, vol 57(3), page 921;

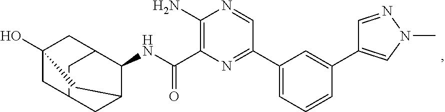 [SMILES: Cn1cc(-c2cccc(-c3cnc(N)c(C(=O)N[C@H]4C5CC6CC4C[C@](O)(C6)C5)n3)c2)cn1]

as described in Forns, P. et al., Bioorg Med Chem Lett 2012, vol. 22(8), page 2784;

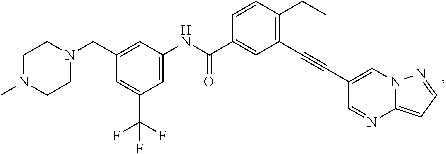 [SMILES: CCc1ccc(C(=O)Nc2cc(CN3CCN(C)CC3)cc(C(F)(F)F)c2)cc1C#Cc1cnc2ccnn2c1]

as described in Gao, M. et al., J Med Chem 2013, vol 56(8), page 3281;

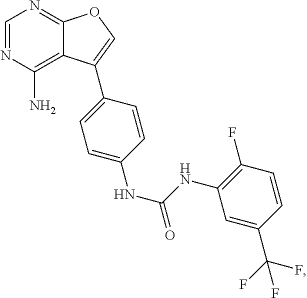 [SMILES: Nc1ncnc2occ(-c3ccc(NC(=O)Nc4cc(C(F)(F)F)ccc4F)cc3)c12]

as described in Harris, P. A. et al., ACS Med Chem Lett 2013, vol. 4(12), page 1238;

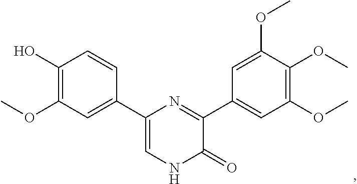

as described in Horbert, R. et al., J Med Chem 2015, vol 58(1), page 170;

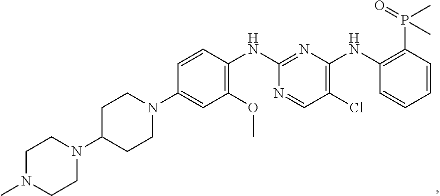

as described in Katayama, R. et al., Proc Natl Acad Sci 2011, vol. 108(18), page 7535;

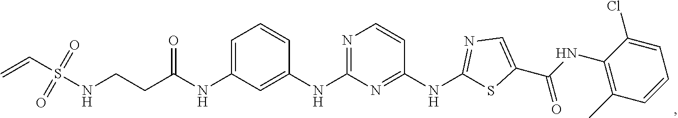 [SMILES: C=CS(=O)(=O)NCCC(=O)Nc1cccc(Nc2nccc(Nc3ncc(C(=O)Nc4c(C)cccc4Cl)s3)n2)c1]

as described in Kwarcinski, F. E. et al., ACS Chem. Biol. 2012, vol. 7(11), page 1910;

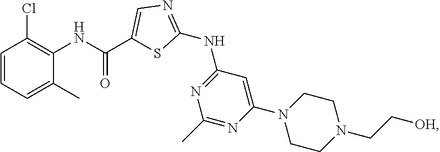 [SMILES: Cc1nc(Nc2ncc(C(=O)Nc3c(C)cccc3Cl)s2)cc(N2CCN(CCO)CC2)n1]

as described in Kwarcinski, F. E. et al., ACS Chem. Biol. 2012, vol. 7(11), page 1910;

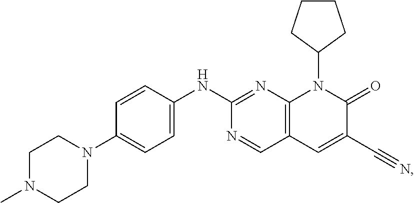 [SMILES: CN1CCN(c2ccc(Nc3ncc4cc(C#N)c(=O)n(C5CCCC5)c4n3)cc2)CC1]

as described in Reddy, M. V. et al., J Med Chem 2014, vol 57(3), page 578.

In certain embodiments, the target protein ligand is an inhibitor and/or binder of Glycogen synthase kinase 3 beta (GSK3b). Inhibitors and/or binders of GSK3b are reported in the literature. Exemplary inhibitors and/or binders of GSK3b include the following compounds:

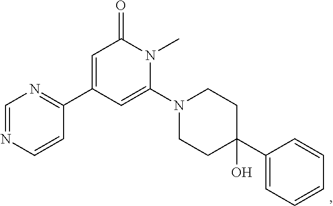 [SMILES: Cn1c(N2CCC(O)(c3ccccc3)CC2)cc(-c2ccncn2)cc1=O]

as described in Coffman, K. et al., Bioorg Med Chem Lett 2011, vol 21(5), page 1429;

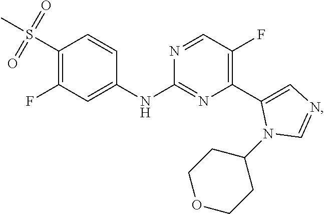 [SMILES: CS(=O)(=O)c1ccc(Nc2ncc(F)c(-c3cncn3C3CCOCC3)n2)cc1F]

as described in Gilmour, P. S. et al., Toxicol Appl Pharmacol 2013, vol 272(2), page 399;

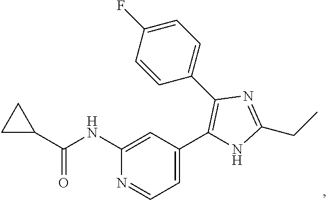 [SMILES: CCc1nc(-c2ccc(F)cc2)c(-c2ccnc(NC(=O)C3CC3)c2)[nH]1]

as described in Heider, F. et al., Eur J Med Chem 2019, vol 175, page 309;

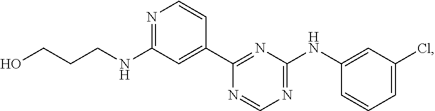 [SMILES: OCCCNc1cc(-c2ncnc(Nc3cccc(Cl)c3)n2)ccn1]

as described in Kuo, G.-H. et al., J Med Chem 2005, vol 48(14), page 4535;

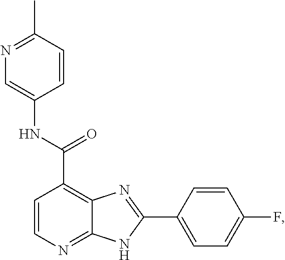 [SMILES: Cc1ccc(NC(=O)c2ccnc3[nH]c(-c4ccc(F)cc4)nc23)cn1]

as described in Lee, S.-C. et al., Bioorg Med Chem Lett 2012, vol 22(13), page 4221;

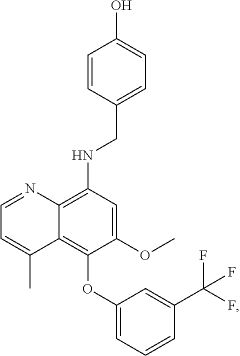 [SMILES: COc1cc(NCc2ccc(O)cc2)c2nccc(C)c2c1Oc1cccc(C(F)(F)F)c1]

as described in Lu, J. et al., Bioorg Med Chem Lett 2014, vol 24(15), page 3392;

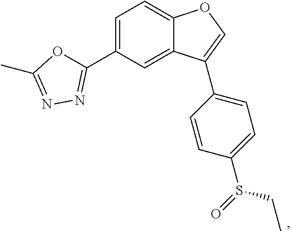 [SMILES: CC[S@](=O)c1ccc(-c2coc3ccc(-c4nnc(C)o4)cc23)cc1]

as described in Luo, G. et al., J Med Chem 2009, vol 52(20), page 6270;

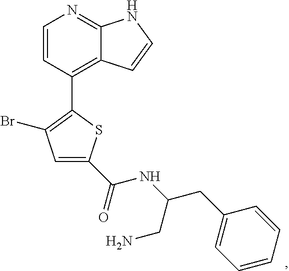 [SMILES: NCC(Cc1ccccc1)NC(=O)c1cc(Br)c(-c2ccnc3[nH]ccc23)s1]

as described in Seefeld, M. A. et al., Bioorg Med Chem Lett 2009, vol 19(8), page 2244;

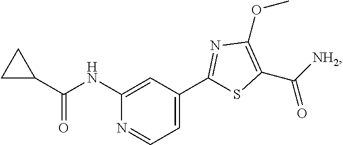 [SMILES: COc1nc(-c2ccnc(NC(=O)C3CC3)c2)sc1C(N)=O]

as described in Sivaprakasam, P. et al., Bioorg Med Chem Lett 2015, vol 25(9), page 1856;

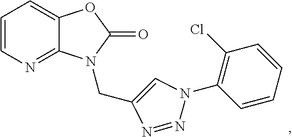 [SMILES: O=c1oc2cccnc2n1Cc1cn(-c2ccccc2Cl)nn1]

as described in Tantray, M. A. et al., Chem Biol Drug Des 2016, vol 87(6), page 918;

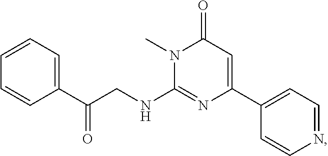 [SMILES: Cn1c(NCC(=O)c2ccccc2)nc(-c2ccncc2)cc1=O]

as described in Uehara, F. et al., Bioorg Med Chem Lett 2013, vol 23(24), page 6928;

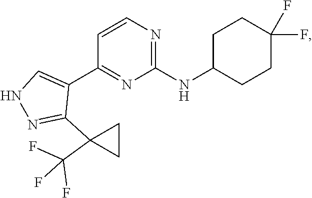 [SMILES: FC1(F)CCC(Nc2nccc(-c3c[nH]nc3C3(C(F)(F)F)CC3)n2)CC1]

as described in Wityak, J. et al., J Med Chem 2015, vol 58(7), page 2967.

In certain embodiments, the target protein ligand is an inhibitor and/or binder of Mouse double minute 2 homolog (MDM2). Inhibitors and/or binders of MDM2 are reported in the literature. Exemplary inhibitors and/or binders of MDM2 include the following compounds:

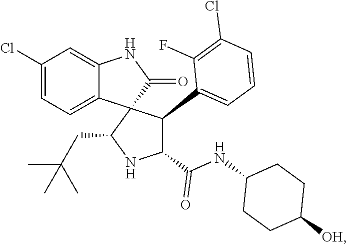 [SMILES: CC(C)(C)C[C@H]1N[C@@H](C(=O)N[C@H]2CC[C@H](O)CC2)[C@H](c2cccc(Cl)c2F)[C@@]12C(=O)Nc1cc(Cl)ccc12]

as described in Wang, S. et al., Cancer Res 2014, vol 74(20), page 5855;

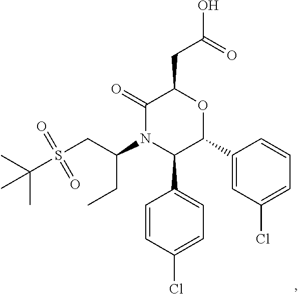 [SMILES: CC[C@@H](CS(=O)(=O)C(C)(C)C)N1C(=O)[C@@H](CC(=O)O)O[C@H](c2cccc(Cl)c2)[C@H]1c1ccc(Cl)cc1]

as described in Gonzalez, A. Z. et al., J Med Chem 2014, vol 57(6), page 2472;

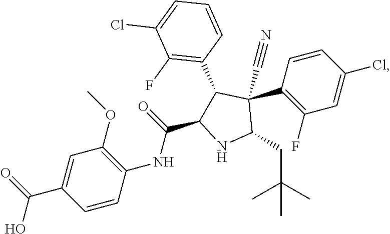 [SMILES: COc1cc(C(=O)O)ccc1NC(=O)[C@@H]1N[C@@H](CC(C)(C)C)[C@](C#N)(c2ccc(Cl)cc2F)[C@H]1c1cccc(Cl)c1F]

as described in Canon, J. et al., Mol Cancer Ther 2015, vol 14(3), page 649;

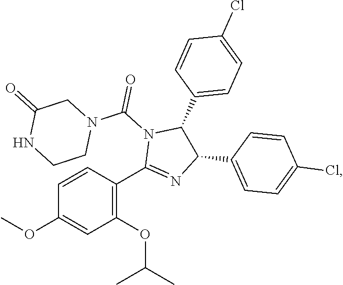 [SMILES: COc1ccc(C2=N[C@@H](c3ccc(Cl)cc3)[C@@H](c3ccc(Cl)cc3)N2C(=O)N2CCNC(=O)C2)c(OC(C)C)c1]

as described in Stachyra-Valat, T. et al., 107th Annu Meet Am Assoc Cancer Res (AACR) (April 16-20, New Orleans) 2016, Abstract 1239;

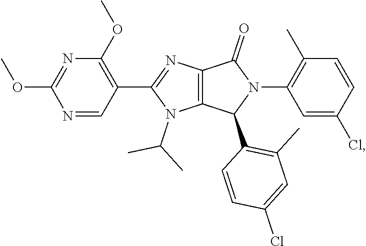 [SMILES: COc1ncc(-c2nc3c(n2C(C)C)[C@H](c2ccc(Cl)cc2C)N(c2cc(Cl)ccc2C)C3=O)c(OC)n1]

as described in Vaupel, A. et al., Bioorg Med Chem Lett 2018, vol 28(20), page 3404;

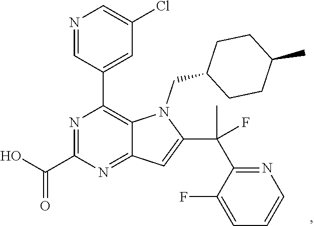 [SMILES: CC(F)(c1ncccc1F)c1cc2nc(C(=O)O)nc(-c3cncc(Cl)c3)c2n1C[C@H]1CC[C@H](C)CC1]

as described in Dinsmore, C. et al., WO 2014/100071;

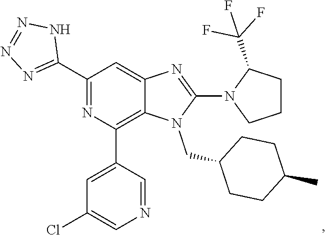 [SMILES: C[C@H]1CC[C@H](Cn2c(N3CCC[C@H]3C(F)(F)F)nc3cc(-c4nnn[nH]4)nc(-c4cncc(Cl)c4)c32)CC1]

as described in Christopher, M. P. et al., US 2014/357618;

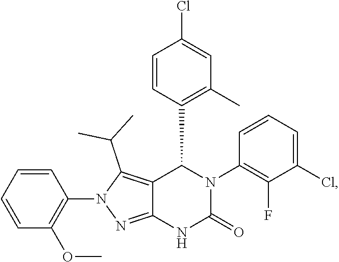 [SMILES: COc1ccccc1-n1nc2c(c1C(C)C)[C@H](c1ccc(Cl)cc1C)N(c1cccc(Cl)c1F)C(=O)N2]

as described in Furet, P. et al., US 2015/353563.

In certain embodiments, the target protein ligand is an inhibitor and/or binder of Signal transducer and activator of transcription 3 (STAT3). Inhibitors and/or binders of STAT3 are reported in the literature. Exemplary inhibitors and/or binders of STAT3 include the following compounds:

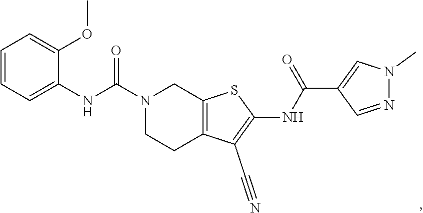 [SMILES: COc1ccccc1NC(=O)N1CCc2c(sc(NC(=O)c3cnn(C)c3)c2C#N)C1]

as described in Chen, Y. et al., CN 108558848;

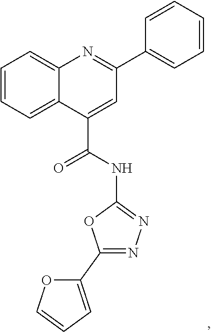 [SMILES: O=C(Nc1nnc(-c2ccco2)o1)c1cc(-c2ccccc2)nc2ccccc12]

as described in Asai, A. et al., WO 2010/004761;

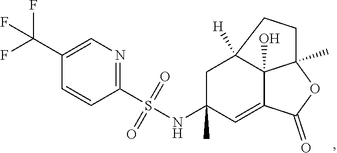

as described in Johansson, M. et al., WO 2018/104295;

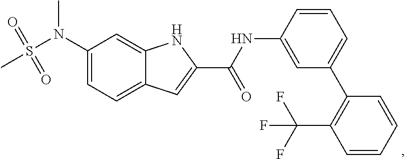 [SMILES: CN(c1ccc2cc(C(=O)Nc3cccc(-c4ccccc4C(F)(F)F)c3)[nH]c2c1)S(C)(=O)=O]

as described in Park, C. H. et al., WO 2014/196793;

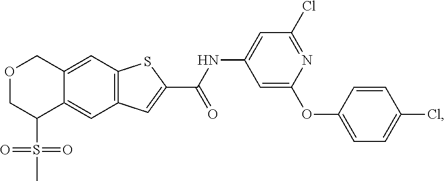 [SMILES: CS(=O)(=O)C1COCc2cc3sc(C(=O)Nc4cc(Cl)nc(Oc5ccc(Cl)cc5)c4)cc3cc21]

as described in Park, C. H. et al., WO 2016/089062;

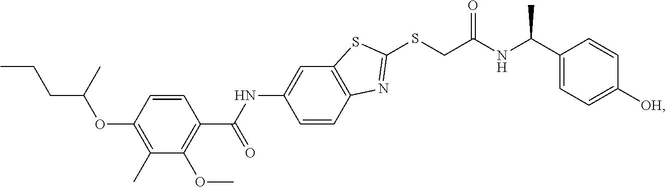 [SMILES: CCCC(C)Oc1ccc(C(=O)Nc2ccc3nc(SCC(=O)N[C@@H](C)c4ccc(O)cc4)sc3c2)c(OC)c1C]

as described in Li, Y. et al., CN

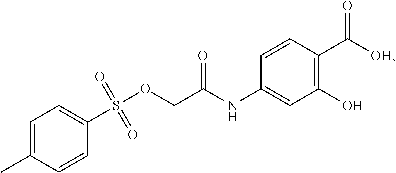 [SMILES: Cc1ccc(S(=O)(=O)OCC(=O)Nc2ccc(C(=O)O)c(O)c2)cc1]

as described in Mallery, S. R. et al., WO 2017/147169;

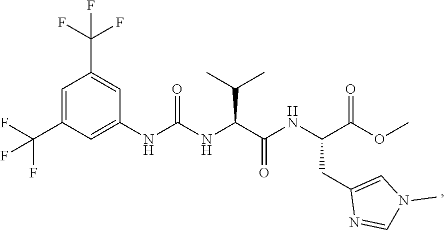 [SMILES: COC(=O)[C@H](Cc1cn(C)cn1)NC(=O)[C@@H](NC(=O)Nc1cc(C(F)(F)F)cc(C(F)(F)F)c1)C(C)C]

as described in Mallery, S. R. et al., WO 2017/147169;

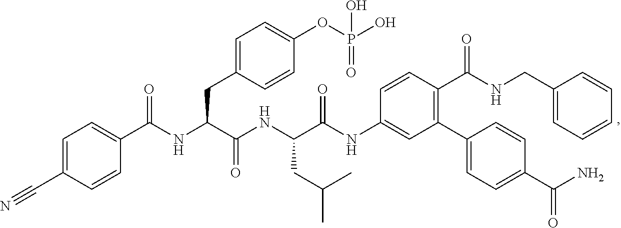 [SMILES: CC(C)C[C@H](NC(=O)[C@H](Cc1ccc(OP(=O)(O)O)cc1)NC(=O)c1ccc(C#N)cc1)C(=O)Nc1ccc(C(=O)NCc2ccccc2)c(-c2ccc(C(N)=O)cc2)c1]

as described in Shahani, V. M. et al., Bioorg Med Chem 2011, vol. 19(5), page 1823;

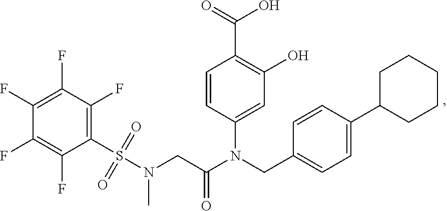 [SMILES: CN(CC(=O)N(Cc1ccc(C2CCCCC2)cc1)c1ccc(C(=O)O)c(O)c1)S(=O)(=O)c1c(F)c(F)c(F)c(F)c1F]

as described in Zhang, X. et al., Proc Natl Acad Sci USA (PNAS) 2012, vol. 109(24), page 9623;

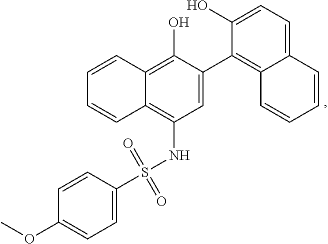 [SMILES: COc1ccc(S(=O)(=O)Nc2cc(-c3c(O)ccc4ccccc34)c(O)c3ccccc23)cc1]

as described in Tweardy. D. J. et al., WO 2015-010107;

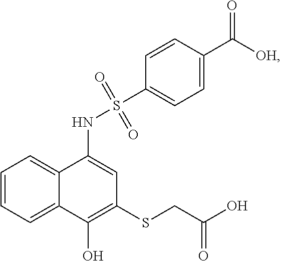 [SMILES: O=C(O)CSc1cc(NS(=O)(=O)c2ccc(C(=O)O)cc2)c2ccccc2c1O]

as described in Bharadwaj, U. et al., Oncotarget 2016, vol. 7(18),

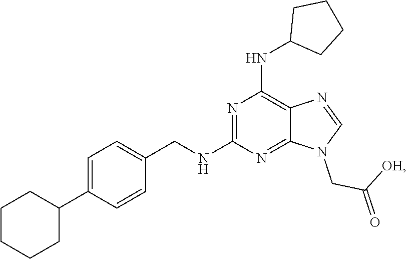 [SMILES: O=C(O)Cn1cnc2c(NC3CCCC3)nc(NCc3ccc(C4CCCCC4)cc3)nc21]

as described in Turkson, J. et al., WO 2011/163424;

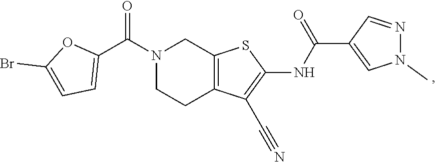 [SMILES: Cn1cc(C(=O)Nc2sc3c(c2C#N)CCN(C(=O)c2ccc(Br)o2)C3)cn1]

as described in Yi, Z. et al., CN108309975.

In certain embodiments, the target protein ligand is an inhibitor and/or binder of Bromodomain-containing protein 4 (BRD4). Inhibitors and/or binders of BRD4 are reported in the literature. Exemplary inhibitors and/or binders of BRD4 include the following compounds:

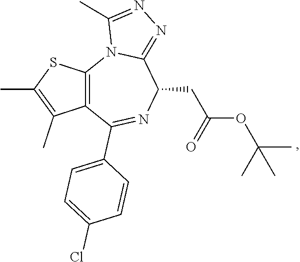 [SMILES: Cc1sc2c(c1C)C(c1ccc(Cl)cc1)=N[C@@H](CC(=O)OC(C)(C)C)c1nnc(C)n1-2]

as described in Bradner, J. E., et al., WO 2011/143669;

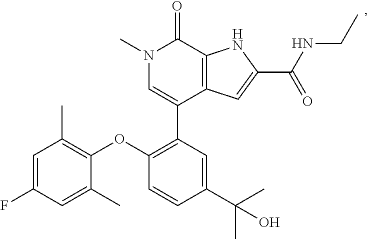 [SMILES: CCNC(=O)c1cc2c(-c3cc(C(C)(C)O)ccc3Oc3c(C)cc(F)cc3C)cn(C)c(=O)c2[nH]1]

as described in Fidanze, S. D., et al., WO 2017/177955;

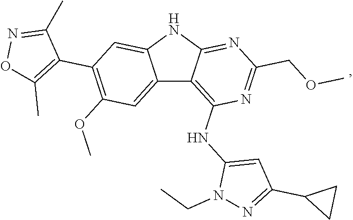 [SMILES: CCn1nc(C2CC2)cc1Nc1nc(COC)nc2[nH]c3cc(-c4c(C)noc4C)c(OC)cc3c12]

as described in Wang, S., et al., WO 2016/138332;

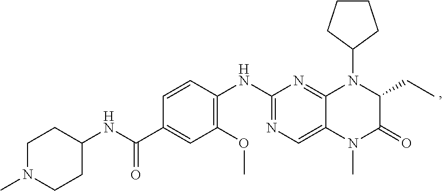 [SMILES: CC[C@@H]1C(=O)N(C)c2cnc(Nc3ccc(C(=O)NC4CCN(C)CC4)cc3OC)nc2N1C1CCCC1]

as described in Chen, L., et al., ACS Med Chem Lett 2015, vol. 6(7), page 764;

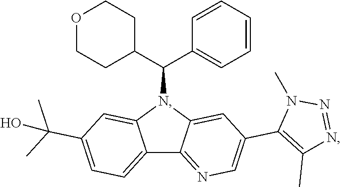 [SMILES: Cc1nnn(C)c1-c1cnc2c3ccc(C(C)(C)O)cc3n([C@H](c3ccccc3)C3CCOCC3)c2c1]

as described in Norris, D. J., et al., WO 2015/100282;

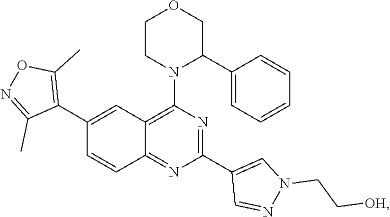 [SMILES: Cc1noc(C)c1-c1ccc2nc(-c3cnn(CCO)c3)nc(N3CCOCC3c3ccccc3)c2c1]

as described in Yang, S. M., et al., Bioorg Med Chem Lett 2018, vol. 28(21), page 3483;

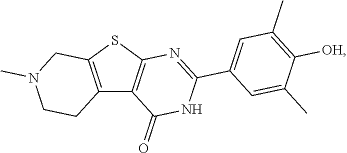 [SMILES: Cc1cc(-c2nc3sc4c(c3c(=O)[nH]2)CCN(C)C4)cc(C)c1O]

as described in Ouyang, L., et al., J Med Chem 2017, vol. 60(24), page 9990;

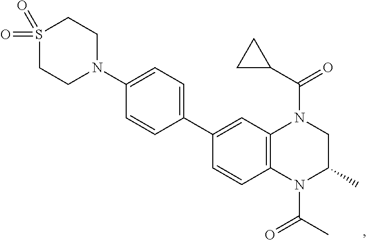 [SMILES: CC(=O)N1c2ccc(-c3ccc(N4CCS(=O)(=O)CC4)cc3)cc2N(C(=O)C2CC2)C[C@@H]1C]

as described in Millan, D. S., et al., ACS Med Chem Lett 2017, vol. 8(8), page 847;

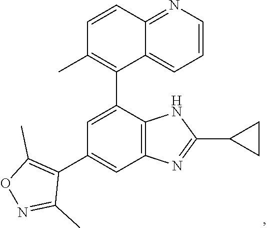 [SMILES: Cc1ccc2ncccc2c1-c1cc(-c2c(C)noc2C)cc2nc(C3CC3)[nH]c12]

as described in Aktoudianakis, E, et al., WO 2014/182929;

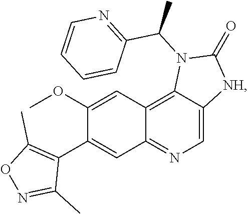 [SMILES: COc1cc2c(cc1-c1c(C)noc1C)ncc1[nH]c(=O)n([C@H](C)c3ccccn3)c12]

as described in Chekler, E. L. P., et al., WO 2017/037567;

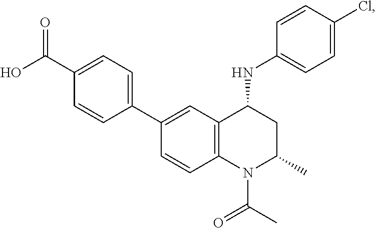 [SMILES: CC(=O)N1c2ccc(-c3ccc(C(=O)O)cc3)cc2[C@H](Nc2ccc(Cl)cc2)C[C@@H]1C]

as described in Demont, E. H., et al., WO 2011/054848;

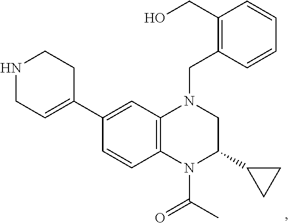 [SMILES: CC(=O)N1c2ccc(C3=CCNCC3)cc2N(Cc2ccccc2CO)C[C@@H]1C1CC1]

as described in Law, R. P. et al., J Med Chem 2018, vol. 61(10), page 4317;

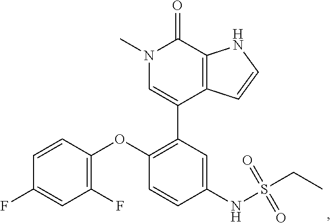 [SMILES: CCS(=O)(=O)Nc1ccc(Oc2ccc(F)cc2F)c(-c2cn(C)c(=O)c3[nH]ccc23)c1]

as described in Hu, Y. et al., WO 2018/086605;

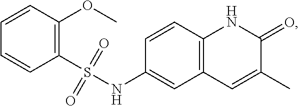 [SMILES: COc1ccccc1S(=O)(=O)Nc1ccc2[nH]c(=O)c(C)cc2c1]

as described in Fish, P. V., et al., WO 2013/027168;

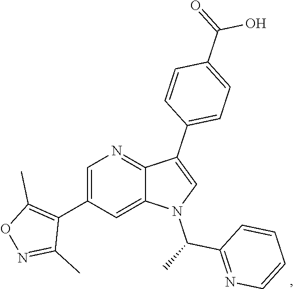 [SMILES: Cc1noc(C)c1-c1cnc2c(-c3ccc(C(=O)O)cc3)cn([C@@H](C)c3ccccn3)c2c1]

as described in Ozer, H. G., et al., Cancer Discov 2018, vol. 8(4), page 458;

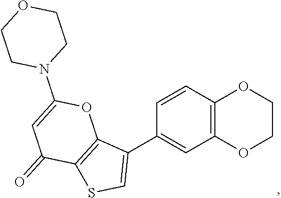 [SMILES: O=c1cc(N2CCOCC2)oc2c(-c3ccc4c(c3)OCCO4)csc12]

as described in Andrews, F. H. et al., Proc Natl Acad Sci USA (PNAS) 2017, vol. 114(7), page E1072;

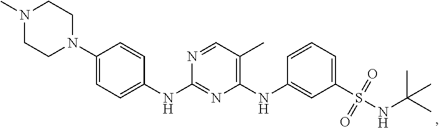 [SMILES: Cc1cnc(Nc2ccc(N3CCN(C)CC3)cc2)nc1Nc1cccc(S(=O)(=O)NC(C)(C)C)c1]

as described in Embe, et al., ACS Chem Biol 2014, vol. 9(5), page 1160;

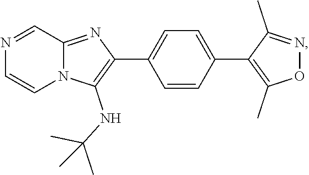 [SMILES: Cc1noc(C)c1-c1ccc(-c2nc3cnccn3c2NC(C)(C)C)cc1]

as described in Marineau, J. J., et al., WO 2015/013635;

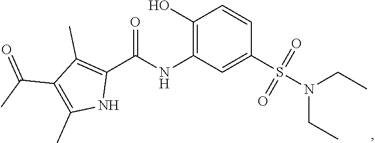 [SMILES: CCN(CC)S(=O)(=O)c1ccc(O)c(NC(=O)c2[nH]c(C)c(C(C)=O)c2C)c1]

as described in Huegle, M., et al., J Med Chem 2016, vol. 59(4), page 1518;

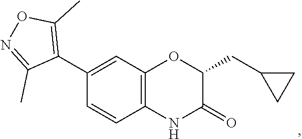 [SMILES: Cc1noc(C)c1-c1ccc2c(c1)O[C@H](CC1CC1)C(=O)N2]

as described in Xue, X. Q., et al., Eur J Med Chem 2018, vol. 152, page 542;

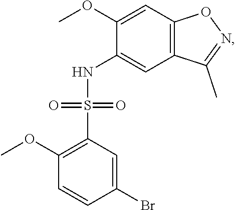 [SMILES: COc1cc2onc(C)c2cc1NS(=O)(=O)c1cc(Br)ccc1OC]

as described in Zhang, M. et al., J Med Chem 2018, vol. 61(7), page 3037;

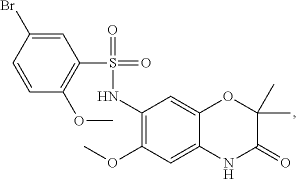 [SMILES: COc1cc2c(cc1NS(=O)(=O)c1cc(Br)ccc1OC)OC(C)(C)C(=O)N2]

as described in Xiang. Q., et al., ACS Med Chem Lett 2018, vol. 9(3), page 262;

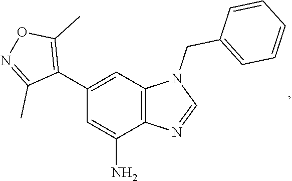 [SMILES: Cc1noc(C)c1-c1cc(N)c2ncn(Cc3ccccc3)c2c1]

as described in Kharenko, O. A., et al., J Med Chem 2018, vol. 61(18), page 8202.

In certain embodiments, the target protein ligand is an inhibitor and/or binder of serine/threonine-protein kinase B-Raf (B-Raf). Inhibitors and/or binders of B-Raf are reported in the literature. Exemplary inhibitors and/or binders of B-Raf include the following compounds:

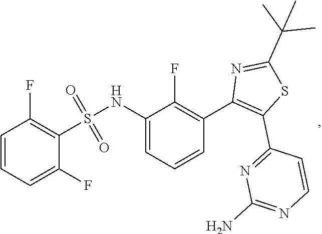 [SMILES: CC(C)(C)c1nc(-c2cccc(NS(=O)(=O)c3c(F)cccc3F)c2F)c(-c2ccnc(N)n2)s1]

as described in Cooke, V. et al., WO 2018/203219;

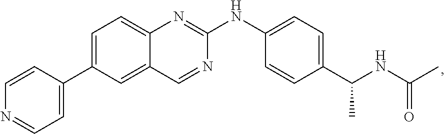 [SMILES: CC(=O)N[C@H](C)c1ccc(Nc2ncc3cc(-c4ccncc4)ccc3n2)cc1]

as described in Aquila, B. et al., WO 2008/020203;

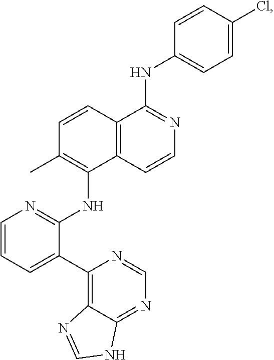 [SMILES: Cc1ccc2c(Nc3ccc(Cl)cc3)nccc2c1Nc1ncccc1-c1ncnc2[nH]cnc12]

as described in Smith, A. L. et al., WO 2008/153947;

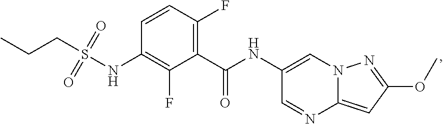 [SMILES: CCCS(=O)(=O)Nc1ccc(F)c(C(=O)Nc2cnc3cc(OC)nn3c2)c1F]

as described in Gradl, S. et al., WO 2011/025951;

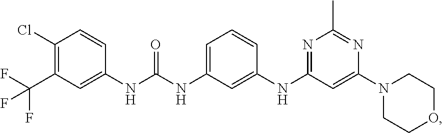 [SMILES: Cc1nc(Nc2cccc(NC(=O)Nc3ccc(Cl)c(C(F)(F)F)c3)c2)cc(N2CCOCC2)n1]

as described in Zhang, Q. et al., WO 2012/139499;

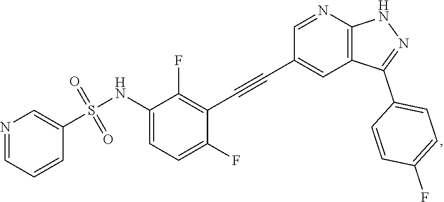 [SMILES: O=S(=O)(Nc1ccc(F)c(C#Cc2cnc3[nH]nc(-c4ccc(F)cc4)c3c2)c1F)c1cccnc1]

as described in Cheng, H. et al., CN105732614;

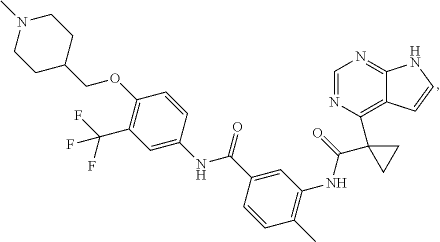 [SMILES: Cc1ccc(C(=O)Nc2ccc(OCC3CCN(C)CC3)c(C(F)(F)F)c2)cc1NC(=O)C1(c2ncnc3[nH]ccc23)CC1]

as described in Chen, Y. et al., CN105801584;

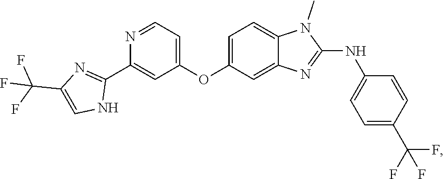 [SMILES: Cn1c(Nc2ccc(C(F)(F)F)cc2)nc2cc(Oc3ccnc(-c4nc(C(F)(F)F)c[nH]4)c3)ccc21]

as described in Sim, T. B. et al., US2013/079343;

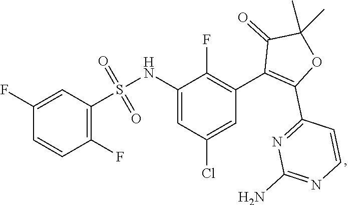 [SMILES: CC1(C)OC(c2ccnc(N)n2)=C(c2cc(Cl)cc(NS(=O)(=O)c3cc(F)ccc3F)c2F)C1=O]

as described in Liu, D. et al., WO 2014/182873;

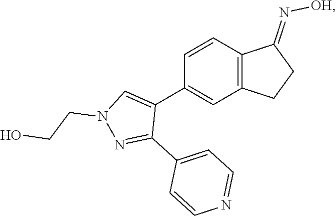 [SMILES: OCCn1cc(-c2ccc3c(c2)CC/C3=N\O)c(-c2ccncc2)n1]

as described in Feng, J. et al., CN103520162;

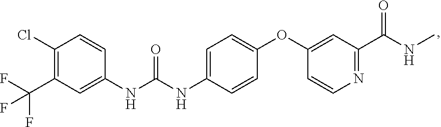 [SMILES: CNC(=O)c1cc(Oc2ccc(NC(=O)Nc3ccc(Cl)c(C(F)(F)F)c3)cc2)ccn1]

as described in Weiss, R. H. et al., WO 2014/007998;

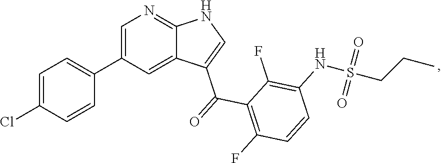 [SMILES: CCCS(=O)(=O)Nc1ccc(F)c(C(=O)c2c[nH]c3ncc(-c4ccc(Cl)cc4)cc23)c1F]

as described in Zhou, C. et al., WO 2014/206343;

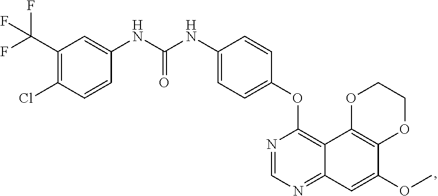 [SMILES: COc1cc2ncnc(Oc3ccc(NC(=O)Nc4ccc(Cl)c(C(F)(F)F)c4)cc3)c2c2c1OCCO2]

as described in Zhang, Q. et al., WO 2018/157730;

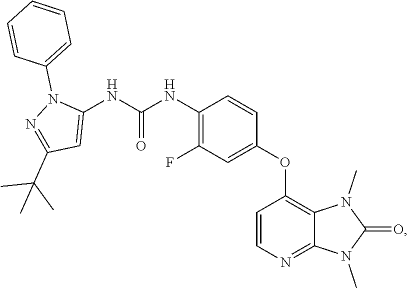 [SMILES: Cn1c(=O)n(C)c2c(Oc3ccc(NC(=O)Nc4cc(C(C)(C)C)nn4-c4ccccc4)c(F)c3)ccnc21]

as described in Springer, C. et al., WO 2011/092469;

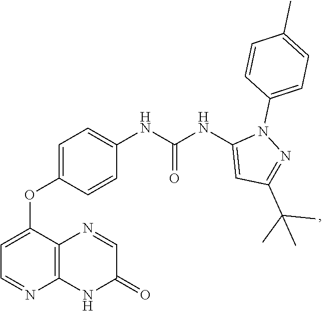 [SMILES: Cc1ccc(-n2nc(C(C)(C)C)cc2NC(=O)Nc2ccc(Oc3ccnc4[nH]c(=O)cnc34)cc2)cc1]

as described in Springer, C. et al., WO/2009/077766;

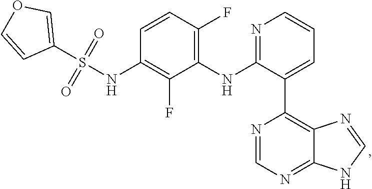 [SMILES: O=S(=O)(Nc1ccc(F)c(Nc2ncccc2-c2ncnc3[nH]cnc23)c1F)c1ccoc1]

as described in Shim, E. K. et al., WO 2012/074249;

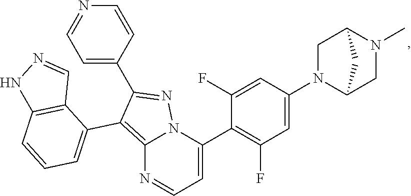 [SMILES: CN1C[C@@H]2C[C@H]1CN2c1cc(F)c(-c2ccnc3c(-c4cccc5[nH]ncc45)c(-c4ccncc4)nn23)c(F)c1]

as described in Levin, J. I. et al., WO 2009/108838.

In certain embodiments, the target protein ligand is an inhibitor and/or binder of serine/threonine-protein kinase C-Raf (C-Raf). Inhibitors and/or binders of C-Raf are reported in the literature. Exemplary inhibitors and/or binders of C-Raf include the following compounds:

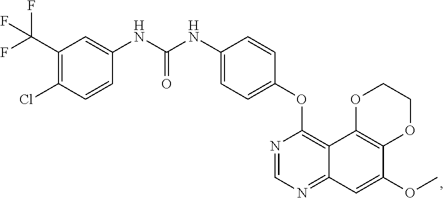 [SMILES: COc1cc2ncnc(Oc3ccc(NC(=O)Nc4ccc(Cl)c(C(F)(F)F)c4)cc3)c2c2c1OCCO2]

as described in Zhang, Q. et al., WO 2018/157730;

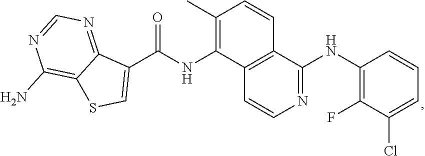 [SMILES: Cc1ccc2c(Nc3cccc(Cl)c3F)nccc2c1NC(=O)c1csc2c(N)ncnc12]

as described in Bae, I. H., et al., WO 2013/100632;

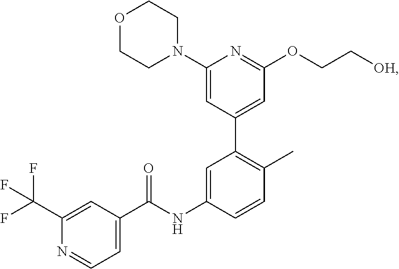 [SMILES: Cc1ccc(NC(=O)c2ccnc(C(F)(F)F)c2)cc1-c1cc(OCCO)nc(N2CCOCC2)c1]

as described in Aversa, R. J., et al., U.S. Pat. No. 9,694,016;

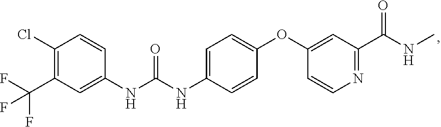 [SMILES: CNC(=O)c1cc(Oc2ccc(NC(=O)Nc3ccc(Cl)c(C(F)(F)F)c3)cc2)ccn1]

as described in Hammock, B. D. et al., WO 2013/100632; 2012/112570;

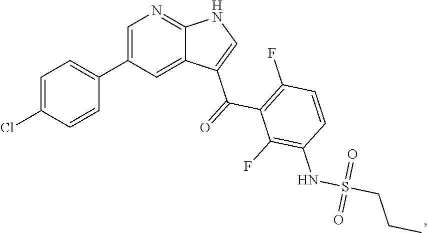 [SMILES: CCCS(=O)(=O)Nc1ccc(F)c(C(=O)c2c[nH]c3ncc(-c4ccc(Cl)cc4)cc23)c1F]

as described in Zhou, C., et al., WO 2014/206343;

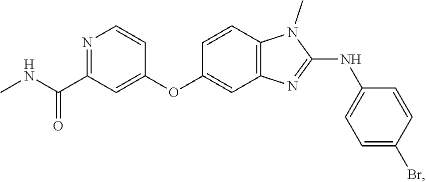 [SMILES: CNC(=O)c1cc(Oc2ccc3c(c2)nc(Nc2ccc(Br)cc2)n3C)ccn1]

as described in Ramurthy, S., et al., J Med Chem 2008, vol. 51(22), page 7049;

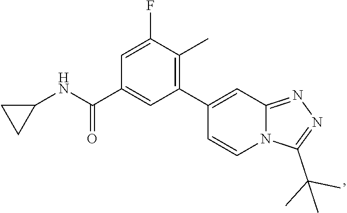 [SMILES: Cc1c(F)cc(C(=O)NC2CC2)cc1-c1ccn2c(C(C)(C)C)nnc2c1]

as described in Aiguade, J., et al., Bioorg Med Chem Lett 2012, vol. 22(10), page 3431;

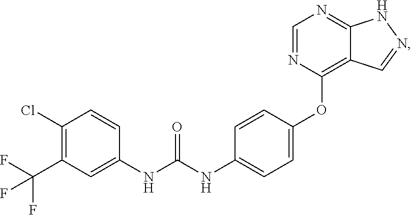 [SMILES: O=C(Nc1ccc(Oc2ncnc3[nH]ncc23)cc1)Nc1ccc(Cl)c(C(F)(F)F)c1]

as described in Yang, L. L. et al., J Med Chem 2013, vol. 56(4), page 1641;

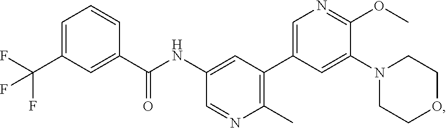 [SMILES: COc1ncc(-c2cc(NC(=O)c3cccc(C(F)(F)F)c3)cnc2C)cc1N1CCOCC1]

as described in Nishiguchi, G. A., et al., J Med Chem 2017, vol. 60(12), page 4869;

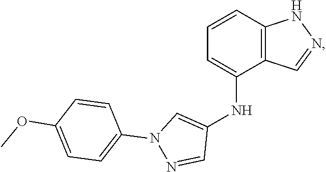 [SMILES: COc1ccc(-n2cc(Nc3cccc4[nH]ncc34)cn2)cc1]

as descrbied by Wang, P. F., et al., Biochem Pharmacol 2017, vol. 132, page 63.

In certain embodiments, the target protein ligand is an inhibitor of Pyruvate Kinase (PKM). Inhibitors of PKM are reported in the literature. Exemplary inhibitors of PKM include the following compounds:

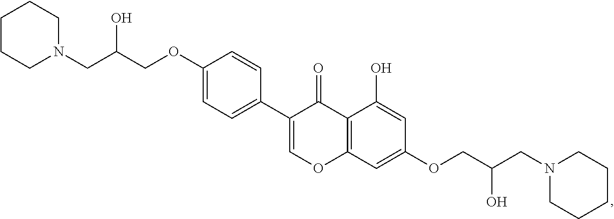

as described in Tao, L., et al., Biochem Pharmacol 2017, vol. 125, page 12;

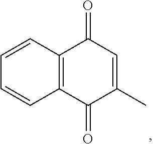

as described in Chen, J., et al., Cancer Lett 2012, vol. 316(2), page 204;

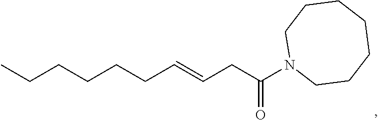 [SMILES: CCCCCC/C=C/CC(=O)N1CCCCCCC1]

as described in Shinohara, H., et al., Cancer Lett 2015, vol. 360(1), page 28;

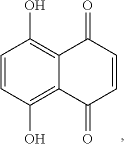 [SMILES: O=C1C=CC(=O)c2c(O)ccc(O)c21]

as described in Hussain, A., et al., Cancer Lett 2016, vol. 374(2), page 250;

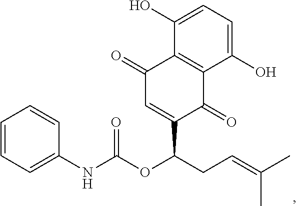 [SMILES: CC(C)=CC[C@@H](OC(=O)Nc1ccccc1)C1=CC(=O)c2c(O)ccc(O)c2C1=O]

as described in Chen, J., et al., CN102526018;

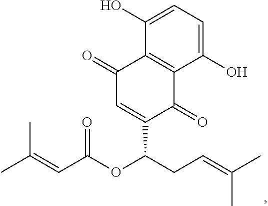 [SMILES: CC(C)=CC[C@H](OC(=O)C=C(C)C)C1=CC(=O)c2c(O)ccc(O)c2C1=O]

as described in Chen, J., et al., CN102552227;

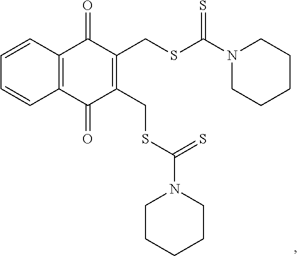 [SMILES: O=C1C(CSC(=S)N2CCCCC2)=C(CSC(=S)N2CCCCC2)C(=O)c2ccccc21]

as described in Yin, Y., et al., CN107226789;

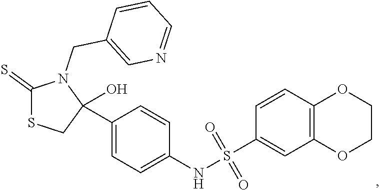 [SMILES: O=S(=O)(Nc1ccc(C2(O)CSC(=S)N2Cc2cccnc2)cc1)c1ccc2c(c1)OCCO2]

as described in Li, R., et al., Eur J Med Chem 2018, vol. 143, page 48;

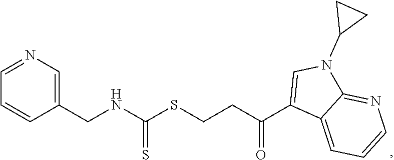 [SMILES: O=C(CCSC(=S)NCc1cccnc1)c1cn(C2CC2)c2ncccc12]

as described in Liu, B., et al., Eur J Med Chem 2019, vol. 170, page 1;

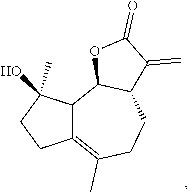

as described in Li, J., et al., J Med Chem 2018, vol. 61(9), page 4155;

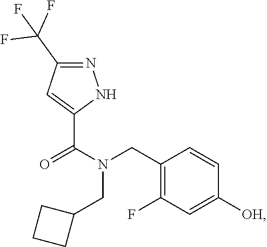 [SMILES: O=C(c1cc(C(F)(F)F)n[nH]1)N(Cc1ccc(O)cc1F)CC1CCC1]

as described in Parnell, K. M., et al., Mol Cancer Ther 2013, vol. 12(8), page 1453;

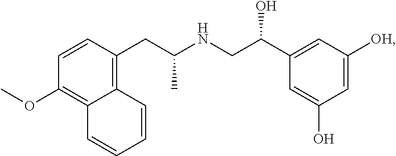 [SMILES: COc1ccc(C[C@@H](C)NC[C@H](O)c2cc(O)cc(O)c2)c2ccccc12]

as described in Singh, N. S., et al., Pharmacol Res 2016, vol. 111, page 757.

In certain embodiments, the target protein ligand is an inducer or inhibitor of Pyruvate Kinase PKLR (PKLR). Inducers or inhibitors of PKLR are reported in the literature. Exemplary inducers or inhibitors of PKLR include the following compounds:

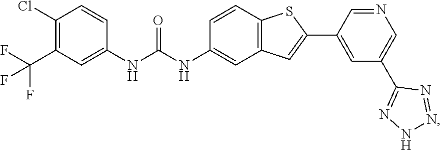 [SMILES: O=C(Nc1ccc(Cl)c(C(F)(F)F)c1)Nc1ccc2sc(-c3cncc(-c4nn[nH]n4)c3)cc2c1]

as described in Boral, S., et al., US 2017/0226094;

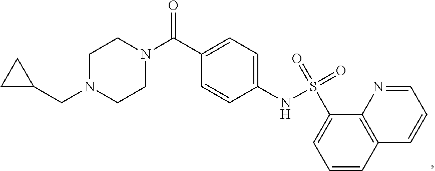 [SMILES: O=C(c1ccc(NS(=O)(=O)c2cccc3cccnc23)cc1)N1CCN(CC2CC2)CC1]

as described in Kung, C., et al., Blood 2017, vol. 130(11), page 1347;

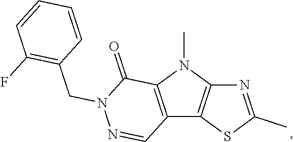 [SMILES: Cc1nc2c(s1)c1cnn(Cc3ccccc3F)c(=O)c1n2C]

as described in Cianchetta, G., et al., WO 2019/035863;

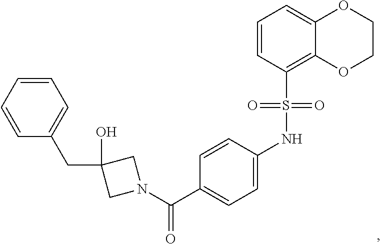 [SMILES: O=C(c1ccc(NS(=O)(=O)c2cccc3c2OCCO3)cc1)N1CC(O)(Cc2ccccc2)C1]

as described in Popovici-Muller, J., et al., WO 2014/074848;

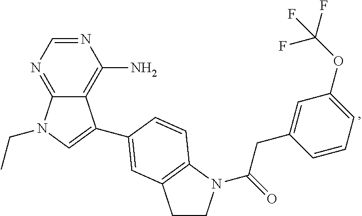 [SMILES: CCn1cc(-c2ccc3c(c2)CCN3C(=O)Cc2cccc(OC(F)(F)F)c2)c2c(N)ncnc21]

as described in Li, Y. S., et al., J Med Chem 2018, vol. 61(24), page 11398;

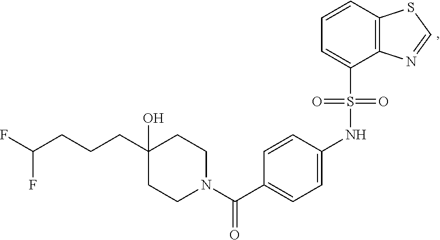 [SMILES: O=C(c1ccc(NS(=O)(=O)c2cccc3scnc23)cc1)N1CCC(O)(CCCC(F)F)CC1]

as described in Cianchetta, G., et al., WO 2014/139144;

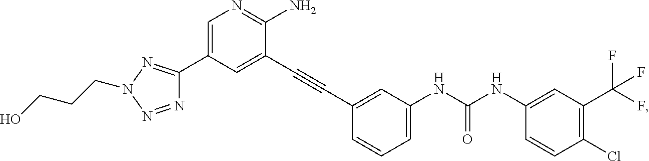 [SMILES: Nc1ncc(-c2nnn(CCCO)n2)cc1C#Cc1cccc(NC(=O)Nc2ccc(Cl)c(C(F)(F)F)c2)c1]

as described in Boral, S., et al., US 2017/0096419;

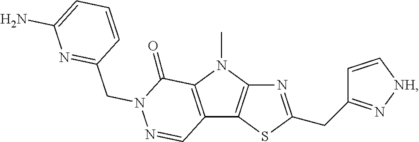 [SMILES: Cn1c2nc(Cc3cc[nH]n3)sc2c2cnn(Cc3cccc(N)n3)c(=O)c21]

as described in Cianchetta, G., et al., WO 2019/035864.

In certain embodiments, the target protein ligand is an inhibitor of TANK Binding Kinase 1 (TBK1). Inhibitors of TBK1 are reported in the literature. Exemplary inhibitors of TBK1 include the following compounds:

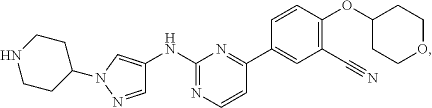 [SMILES: N#Cc1cc(-c2ccnc(Nc3cnn(C4CCNCC4)c3)n2)ccc1OC1CCOCC1]

as described in Hoelzemann, G. et al., WO 2013/034238;

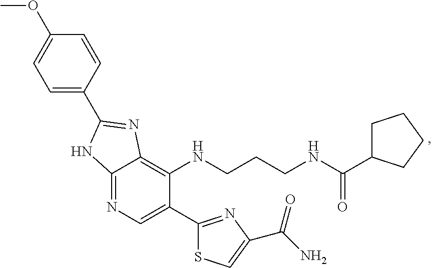 [SMILES: COc1ccc(-c2nc3c(NCCCNC(=O)C4CCCC4)c(-c4nc(C(N)=O)cs4)cnc3[nH]2)cc1]

as described in Johannes, J. W. et al., Bioorg Med Chem Lett 2014, vol 24(4), page 1138;

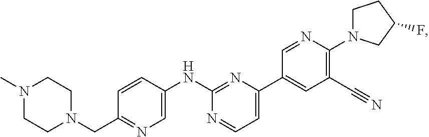 [SMILES: CN1CCN(Cc2ccc(Nc3nccc(-c4cnc(N5CC[C@H](F)C5)c(C#N)c4)n3)cn2)CC1]

as described in Newton, G. K. et al., WO 2018/154315;

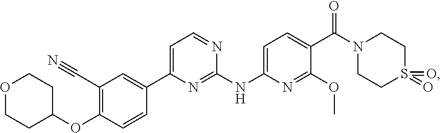 [SMILES: COc1nc(Nc2nccc(-c3ccc(OC4CCOCC4)c(C#N)c3)n2)ccc1C(=O)N1CCS(=O)(=O)CC1]

as described in Karra, S. R. et al., WO 2019/079373;

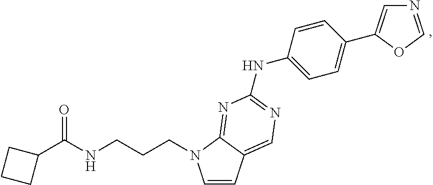 [SMILES: O=C(NCCCn1ccc2cnc(Nc3ccc(-c4cnco4)cc3)nc21)C1CCC1]

as described in McIver, E. G. et al., WO 2010/100431;

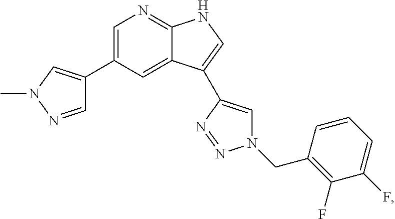 [SMILES: Cn1cc(-c2cnc3[nH]cc(-c4cn(Cc5cccc(F)c5F)nn4)c3c2)cn1]

as described in Dorsch, D. et al., WO 2010/127754;

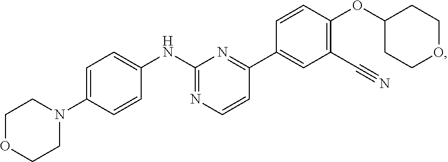 [SMILES: N#Cc1cc(-c2ccnc(Nc3ccc(N4CCOCC4)cc3)n2)ccc1OC1CCOCC1]

as described in Holcomb, R. et al., WO 2011/046970;

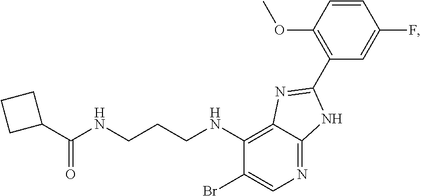 [SMILES: COc1ccc(F)cc1-c1nc2c(NCCCNC(=O)C3CCC3)c(Br)cnc2[nH]1]

as described in Wang, T. et al., Bioorg Med Chem Lett 2012, vol 22(5), page 2063;

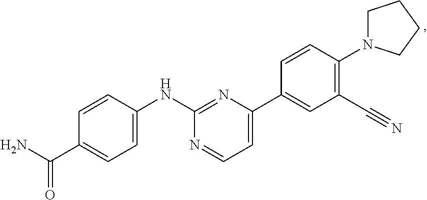 [SMILES: N#Cc1cc(-c2ccnc(Nc3ccc(C(N)=O)cc3)n2)ccc1N1CCCC1]

as described in Perrior, T. R. et al., WO 2012/010826;

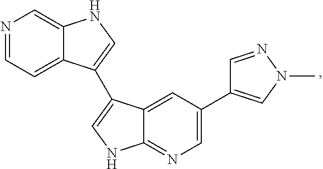 [SMILES: Cn1cc(-c2cnc3[nH]cc(-c4c[nH]c5cnccc45)c3c2)cn1]

as described in Dorsch, D. et al., WO 2012/104007;

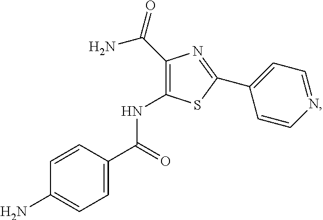 [SMILES: NC(=O)c1nc(-c2ccncc2)sc1NC(=O)c1ccc(N)cc1]

as described in Karra, S. R. et al., WO 2012/161879;

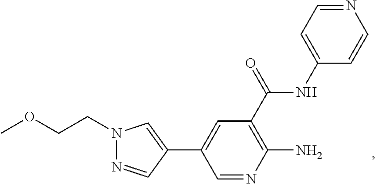 [SMILES: COCCn1cc(-c2cnc(N)c(C(=O)Nc3ccncc3)c2)cn1]

as described in Hoelzemann, G. et al., WO 2012/161877;

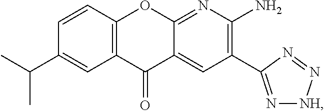 [SMILES: CC(C)c1ccc2oc3nc(N)c(-c4nn[nH]n4)cc3c(=O)c2c1]

as described in Beyett, T. S. et al., Mol Pharmacol 2018, vol. 94(4), page 1210;

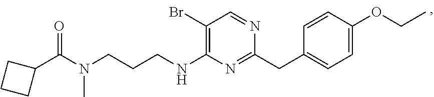 [SMILES: CCOc1ccc(Cc2ncc(Br)c(NCCCN(C)C(=O)C3CCC3)n2)cc1]

as described in Crew, A. P. et al., J Med Chem 2018, volume 61, page 583;

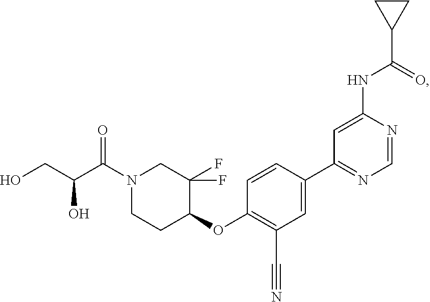 [SMILES: N#Cc1cc(-c2cc(NC(=O)C3CC3)ncn2)ccc1O[C@H]1CCN(C(=O)[C@@H](O)CO)CC1(F)F]

as described in Du, Z. et al., WO 2017/106556;

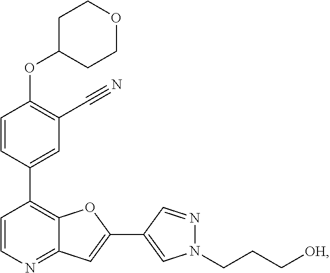 [SMILES: N#Cc1cc(-c2ccnc3cc(-c4cnn(CCCO)c4)oc23)ccc1OC1CCOCC1]

as described in Eggenweiler, H. et al., WO 2013/117285;

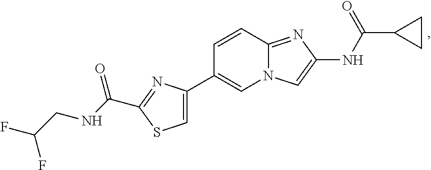 [SMILES: O=C(NCC(F)F)c1nc(-c2ccc3nc(NC(=O)C4CC4)cn3c2)cs1]

as described in Keung, W. et al., WO 2015/134171;

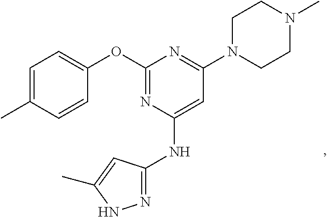

as described in Richters, A. et al., ACS Chem Biol 2015, vol. 10(1), page 289.

In certain embodiments, the target protein ligand is a binder of Microtubule-Associated Protein Tau (MAPT; Tau). Binders of Tau are reported in the literature. Exemplary binders of Tau include the following compounds:

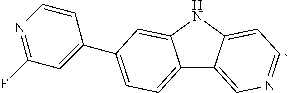 [SMILES: Fc1cc(-c2ccc3c(c2)[nH]c2ccncc23)ccn1]

as described in Berndt, M et al., WO 2019/145291;

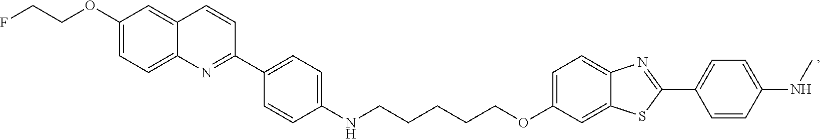 [SMILES: CNc1ccc(-c2nc3ccc(OCCCCCNc4ccc(-c5ccc6cc(OCCF)ccc6n5)cc4)cc3s2)cc1]

as described in Kong, Y. et al., 63rd Annu Meet Soc Nucl Med Mol Imaging (SNMMI) (June 11-15, San Diego) 2016, Abstract 1030;

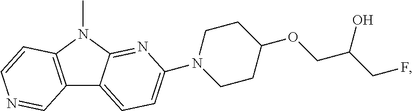 [SMILES: Cn1c2ccncc2c2ccc(N3CCC(OCC(O)CF)CC3)nc21]

as described in EP3118202;

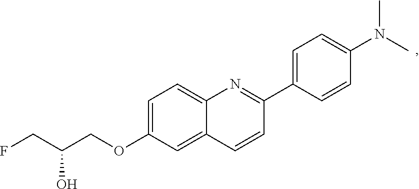 [SMILES: CN(C)c1ccc(-c2ccc3cc(OC[C@H](O)CF)ccc3n2)cc1]

as described in Kudo, Y. et al., WO 2015/060365;

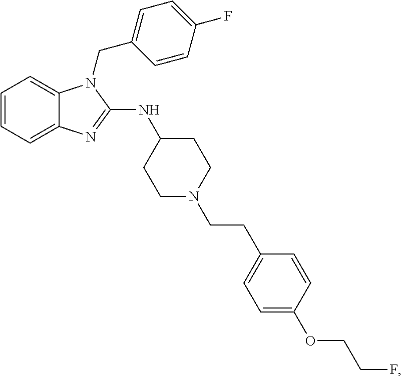 [SMILES: FCCOc1ccc(CCN2CCC(Nc3nc4ccccc4n3Cc3ccc(F)cc3)CC2)cc1]

as described in Riss, P. J et al., Med Chem Commun 2013, vol. 4(5), page 852.

In certain embodiments, the target protein ligand inhibits Protein Kinase B alpha (Akt1). Inhibitors of AKT1 are reported in the literature. Exemplary inhibitors of AKT1 include the following compounds:

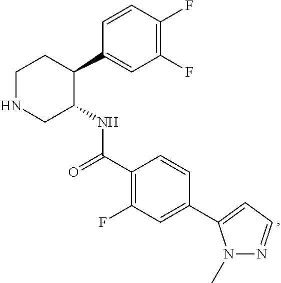 [SMILES: Cn1nccc1-c1ccc(C(=O)N[C@@H]2CNCC[C@H]2c2ccc(F)c(F)c2)c(F)c1]

as described in Dong, X et al., WO 2018/137555;

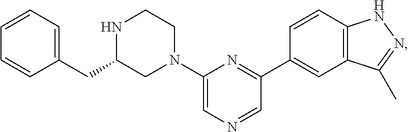 [SMILES: Cc1n[nH]c2ccc(-c3cncc(N4CCN[C@@H](Cc5ccccc5)C4)n3)cc12]

as described by Chan, T. et al., WO 2007/126964;

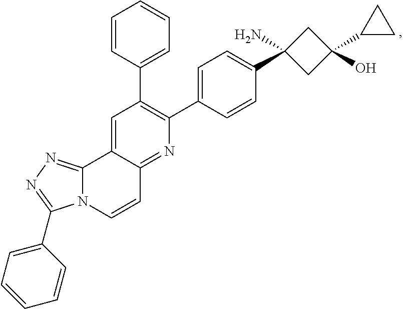 [SMILES: N[C@]1(c2ccc(-c3nc4ccn5c(-c6ccccc6)nnc5c4cc3-c3ccccc3)cc2)C[C@](O)(C2CC2)C1]

as described in Furuyama, H. et al., WO 2018/137555;

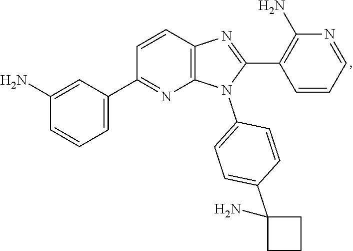 [SMILES: Nc1cccc(-c2ccc3nc(-c4cccnc4N)n(-c4ccc(C5(N)CCC5)cc4)c3n2)c1]

as described in Ashwell, M. A. et al., WO 2011/172203;

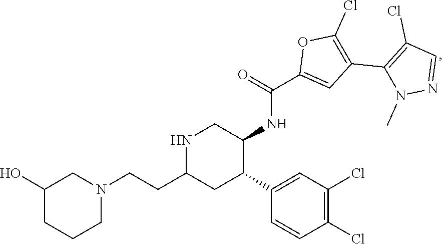 [SMILES: Cn1ncc(Cl)c1-c1cc(C(=O)N[C@@H]2CNC(CCN3CCCC(O)C3)C[C@H]2c2ccc(Cl)c(Cl)c2)oc1Cl]

as described in Dong, X. et al., WO 2015/144021;

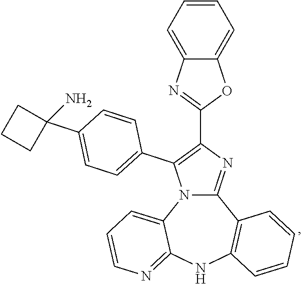 [SMILES: NC1(c2ccc(-c3c(-c4nc5ccccc5o4)nc4n3-c3cccnc3Nc3ccccc3-4)cc2)CCC1]

as described in Ashwell, M. A. et al., WO 2012/061342;

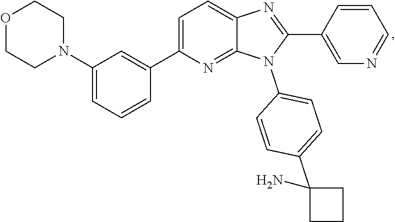 [SMILES: NC1(c2ccc(-n3c(-c4cccnc4)nc4ccc(-c5cccc(N6CCOCC6)c5)nc43)cc2)CCC1]

as described in Ashwell, M. A. et al., WO 2012/177852;

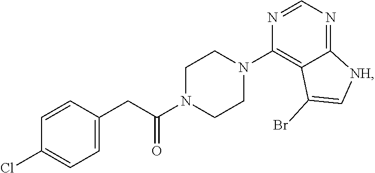 [SMILES: O=C(Cc1ccc(Cl)cc1)N1CCN(c2ncnc3[nH]cc(Br)c23)CC1]

as described in Ai, W. et al., CN 104876933;

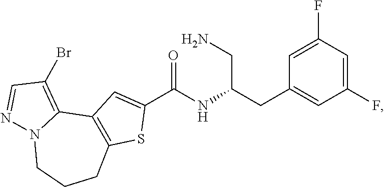 [SMILES: NC[C@H](Cc1cc(F)cc(F)c1)NC(=O)c1cc2c(s1)CCCn1ncc(Br)c1-2]

as described in Lu, L. et al., WO 2017/215588;

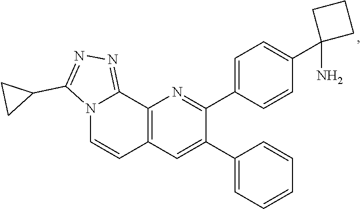 [SMILES: NC1(c2ccc(-c3nc4c(ccn5c(C6CC6)nnc45)cc3-c3ccccc3)cc2)CCC1]

as described in Fan, W. et al., WO 2010/104933;

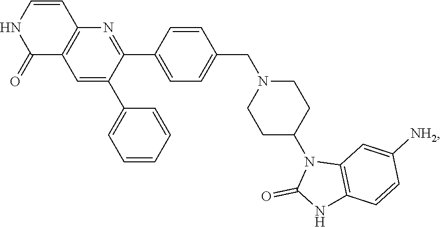 [SMILES: Nc1ccc2[nH]c(=O)n(C3CCN(Cc4ccc(-c5nc6cc[nH]c(=O)c6cc5-c5ccccc5)cc4)CC3)c2c1]

as described in Weisner, J., et al., Angew. Chem. Int. Ed. 2015, 54, 10313.

In certain embodiments, the target protein ligand inhibits Protein Kinase B gamma (Akt3). Inhibitors of AKT3 are reported in the literature. Exemplary inhibitors of AKT3 include the following compounds:

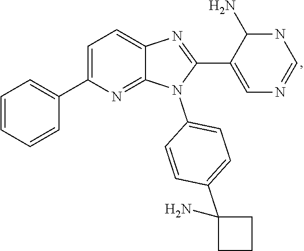 [SMILES: NC1NC=NC=C1c1nc2ccc(-c3ccccc3)nc2n1-c1ccc(C2(N)CCC2)cc1]

as described in Lapierre, J. M. et al., J Med Chem 2016, vol. 59(13), page 6455;

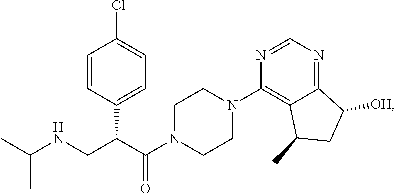 [SMILES: CC(C)NC[C@@H](C(=O)N1CCN(c2ncnc3c2[C@H](C)C[C@H]3O)CC1)c1ccc(Cl)cc1]

as described in Blake, J. F. et al., J Med Chem 2012, vol. 55(18), page 8110;

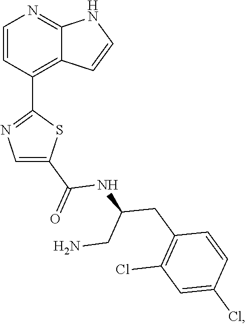 [SMILES: NC[C@H](Cc1ccc(Cl)cc1Cl)NC(=O)c1cnc(-c2ccnc3[nH]ccc23)s1]

as described in Chang, S. et al., Bioorg Med Chem Lett 2012, vol. 22(2), page 1208;

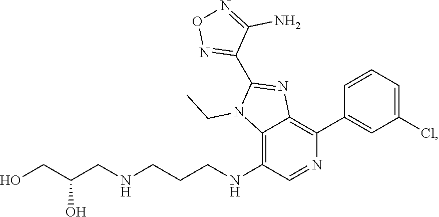 [SMILES: CCn1c(-c2nonc2N)nc2c(-c3cccc(Cl)c3)ncc(NCCCNC[C@H](O)CO)c21]

as described in Clark, J. T. et al., WO 2005/011700;

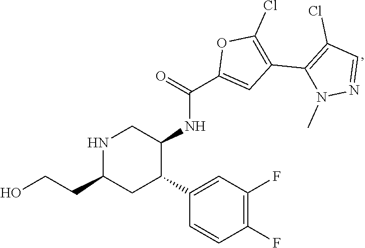 [SMILES: Cn1ncc(Cl)c1-c1cc(C(=O)N[C@@H]2CN[C@H](CCO)C[C@H]2c2ccc(F)c(F)c2)oc1Cl]

as described in Dong, X. et al., J Med Chem 2019, vol. 62(15), page 7264;

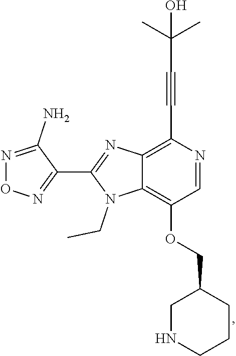 [SMILES: CCn1c(-c2nonc2N)nc2c(C#CC(C)(C)O)ncc(OC[C@H]3CCCNC3)c21]

as described in Heerding, D. A. et al., J Med Chem 2008, vol. 51(18), page 5663;

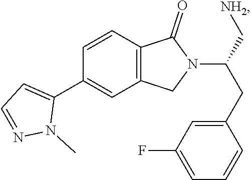 [SMILES: Cn1nccc1-c1ccc2c(c1)CN([C@H](CN)Cc1cccc(F)c1)C2=O]

as described in Huang, T. et al., WO 2013/056015;

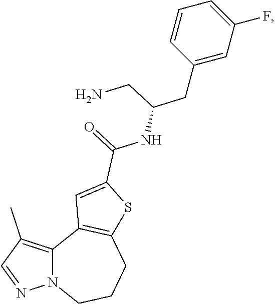 [SMILES: Cc1cnn2c1-c1cc(C(=O)N[C@H](CN)Cc3cccc(F)c3)sc1CCC2]

as described in Li, G. et al., WO 2019/114741;

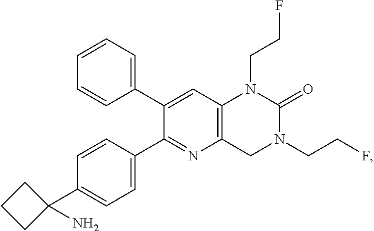 [SMILES: NC1(c2ccc(-c3nc4c(cc3-c3ccccc3)N(CCF)C(=O)N(CCF)C4)cc2)CCC1]

as described in Zhang, L. et al., WO 2011/077098;

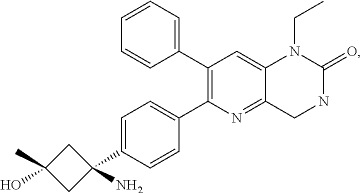 [SMILES: CCN1C(=O)NCc2nc(-c3ccc([C@]4(N)C[C@](C)(O)C4)cc3)c(-c3ccccc3)cc21]

as described in Zhang, L. et al., WO 2011/077098.

In certain embodiments, the target protein ligand is an inhibitor of ribosomal protein S6 kinase alpha-3 (RSK2). Inhibitors of RSK2 are reported in the literature. Exemplary inhibitors of RSK2 include the following compounds:

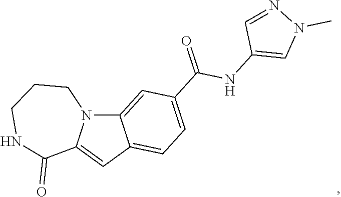 [SMILES: Cn1cc(NC(=O)c2ccc3cc4n(c3c2)CCCNC4=O)cn1]

as described in Boyer, S. J. et al., Bioorg. Med. Chem. Lett. 2012, vol. 22(1), page 733;

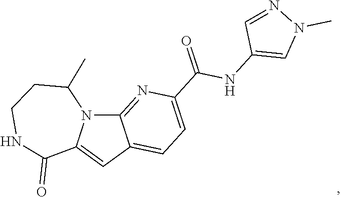 [SMILES: CC1CCNC(=O)c2cc3ccc(C(=O)Nc4cnn(C)c4)nc3n21]

as described in Boyer, S. J. et al., WO 2011/071725;

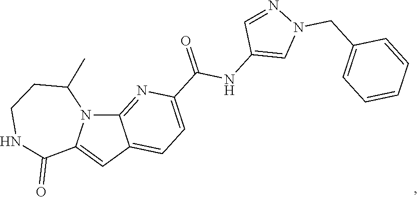 [SMILES: CC1CCNC(=O)c2cc3ccc(C(=O)Nc4cnn(Cc5ccccc5)c4)nc3n21]

as described in Boyer, S. J. et al., WO 2011/071725;

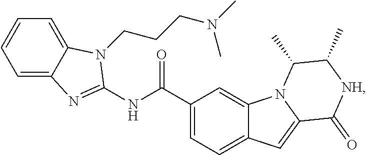 [SMILES: C[C@@H]1NC(=O)c2cc3ccc(C(=O)Nc4nc5ccccc5n4CCCN(C)C)cc3n2[C@@H]1C]

as described in Boyer, S. J. et al., WO 2011/071716;

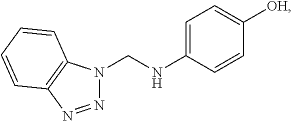 [SMILES: Oc1ccc(NCn2nnc3ccccc32)cc1]

as described in Cao, X. et al., CN 102688233;

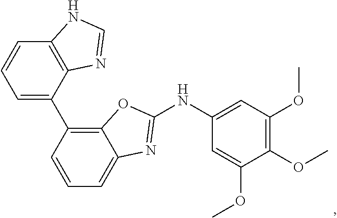 [SMILES: COc1cc(Nc2nc3cccc(-c4cccc5[nH]cnc45)c3o2)cc(OC)c1OC]

as described in Costales, A. et al., Bioorg. Med. Chem. Lett. 2014, vol. 24(6), page 1592;

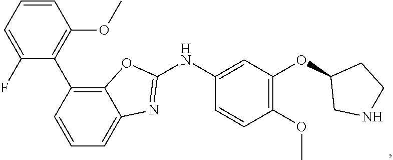 [SMILES: COc1ccc(Nc2nc3cccc(-c4c(F)cccc4OC)c3o2)cc1O[C@H]1CCNC1]

as described in Costales, A. et al., Bioorg. Med. Chem. Lett. 2014, vol. 24(6), page 1592;

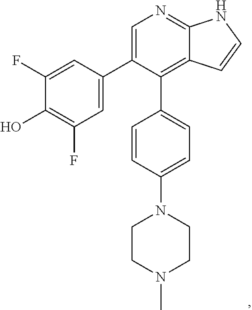 [SMILES: CN1CCN(c2ccc(-c3c(-c4cc(F)c(O)c(F)c4)cnc4[nH]ccc34)cc2)CC1]

as described in Jain, R. et al., Bioorg. Med. Chem. Lett. 2018, vol. 28(19)page 319

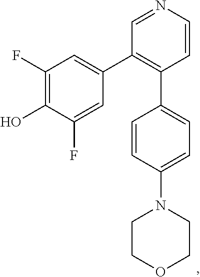 [SMILES: Oc1c(F)cc(-c2cnccc2-c2ccc(N3CCOCC3)cc2)cc1F]

as described in Jain, R. et al., J Med Chem 2015, vol. 58(17). page 6766.

In certain embodiments, the target protein ligand is an inhibitor of ribosomal protein S6 kinase alpha-2 (RSK3). Inhibitors of RSK3 are reported in the literature. Exemplary inhibitors of RSK3 include the following compounds:

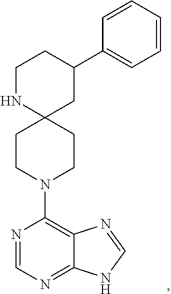 [SMILES: c1ccc(C2CCNC3(CCN(c4ncnc5[nH]cnc45)CC3)C2)cc1]

as described in Allen, C. E. et al., Bioorg. Med. Chem. 2013, vol. 21(18), page 5707;

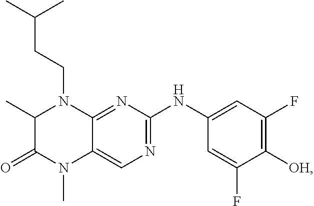 [SMILES: CC(C)CCN1c2nc(Nc3cc(F)c(O)c(F)c3)ncc2N(C)C(=O)C1C]

as described in Ciceri, P. et al., Nat Chem Biol 2014, vol. 10(4), page 305;

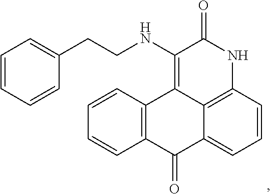 [SMILES: O=C1c2ccccc2-c2c(NCCc3ccccc3)c(=O)[nH]c3cccc1c23]

as described in Fomina-Yadlin, D. et al., Proc Natl Acad Sci USA (PNAS) 2010, vol. 107(34), page 15099.

In certain embodiments, the target protein ligand is a binder and/or activator of glycogen synthase (GYS1/2). Binders and/or activators of GYS are reported in the literature. Exemplary binders and/or activators of GYS include the following compounds:

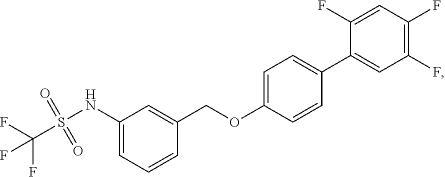 [SMILES: O=S(=O)(Nc1cccc(COc2ccc(-c3cc(F)c(F)cc3F)cc2)c1)C(F)(F)F]

as described in Bolin, D. R. et al., WO 2011/067174;

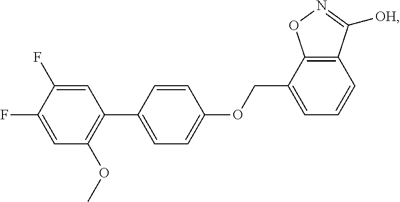 [SMILES: COc1cc(F)c(F)cc1-c1ccc(OCc2cccc3c(O)noc23)cc1]

as described in Bolin, D. R. et al., WO 2011/057956;

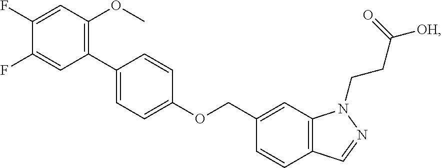 [SMILES: COc1cc(F)c(F)cc1-c1ccc(OCc2ccc3cnn(CCC(=O)O)c3c2)cc1]

as described in Bolin, D. R. et al., WO 2011/057959;

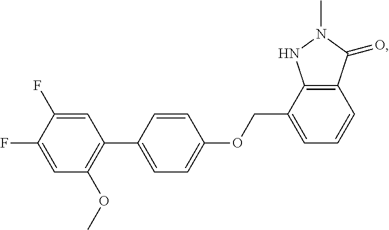 [SMILES: COc1cc(F)c(F)cc1-c1ccc(OCc2cccc3c(=O)n(C)[nH]c23)cc1]

as described in Bolin, D. R. et al., WO 2011/057993;

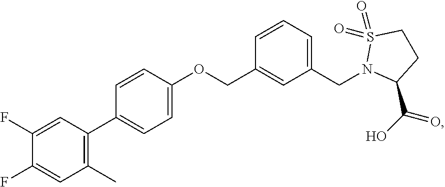 [SMILES: Cc1cc(F)c(F)cc1-c1ccc(OCc2cccc(CN3[C@H](C(=O)O)CCS3(=O)=O)c2)cc1]

as described in Bolin, D. R. et al., WO 2011/067266;

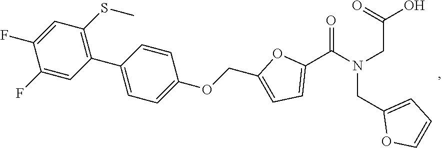 [SMILES: CSc1cc(F)c(F)cc1-c1ccc(OCc2ccc(C(=O)N(CC(=O)O)Cc3ccco3)o2)cc1]

as described in Miyanaga, W. et al., WO 2016/002853;

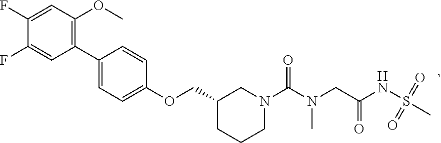 [SMILES: COc1cc(F)c(F)cc1-c1ccc(OC[C@H]2CCCN(C(=O)N(C)CC(=O)NS(C)(=O)=O)C2)cc1]

as described in Yun, W., WO 2011/058122.

In certain embodiments, the target protein ligand is an inhibitor and/or binder of Huntingtin (HTT). Inhibitors and/or binders of HTT are reported in the literature. Exemplary inhibitors and/or binders of HTT include the following compounds:

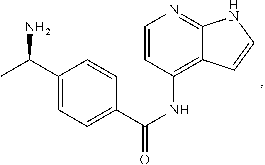 [SMILES: C[C@@H](N)c1ccc(C(=O)Nc2ccnc3[nH]ccc23)cc1]

as described in Bard, J. et al., J Biomol Screen 2014, vol. 19(2), page 191;

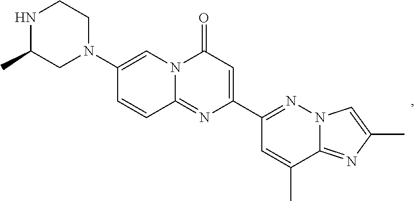

as described in Bhattacharyya, A. et al., WO 2019/005993;

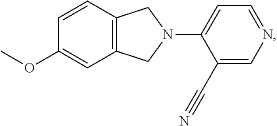 [SMILES: COc1ccc2c(c1)CN(c1ccncc1C#N)C2]

as described in Dominguez, C. et al., WO 2016/033436.

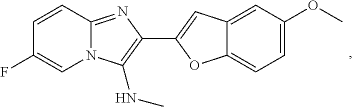 [SMILES: CNc1c(-c2cc3cc(OC)ccc3o2)nc2ccc(F)cn12]

as described in Dominguez, C. et al., WO 2016/033440;

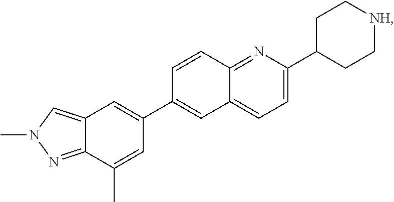 [SMILES: Cc1cc(-c2ccc3nc(C4CCNCC4)ccc3c2)cc2cn(C)nc12]

as described in Woll, M. G. et al., WO 2018/226622.

In certain embodiments, the target protein ligand is an inhibitor and/or binder of Mammalian Target of Rapamycin (mTOR). Inhibitors and/or binders of mTOR are reported in the literature. Exemplary inhibitors and/or binders of mTOR include the following compounds:

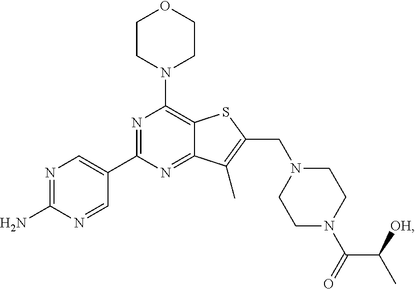 [SMILES: Cc1c(CN2CCN(C(=O)[C@H](C)O)CC2)sc2c(N3CCOCC3)nc(-c3cnc(N)nc3)nc12]

as described in Derynck, M. K. et al., WO 2012/164060;

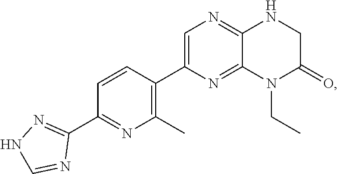 [SMILES: CCN1C(=O)CNc2ncc(-c3ccc(-c4nc[nH]n4)nc3C)nc21]

as described in Xu, S. et al., WO 2013/138557, Raymon, H. et al., WO 2014/172424 and WO 2014/172425;

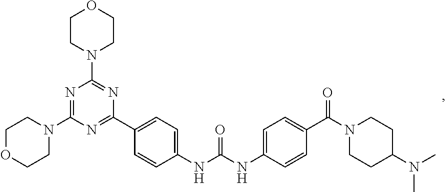 [SMILES: CN(C)C1CCN(C(=O)c2ccc(NC(=O)Nc3ccc(-c4nc(N5CCOCC5)nc(N5CCOCC5)n4)cc3)cc2)CC1]

as described in Venkatesan, A. M. et al., US2017/224696;

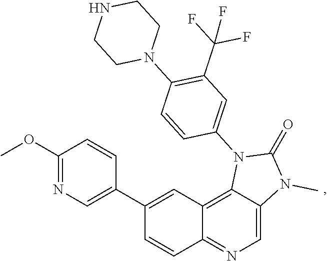

as described in Wu, F. et al., WO 2013/071698;

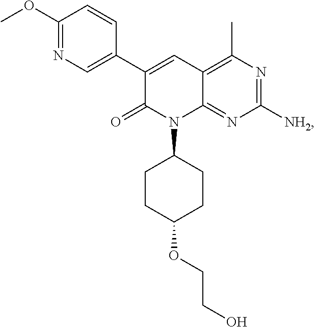 [SMILES: COc1ccc(-c2cc3c(C)nc(N)nc3n([C@H]3CC[C@H](OCCO)CC3)c2=O)cn1]

as described in Yu, C. et al., US2014/038991;

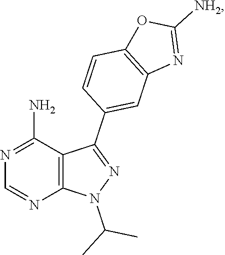 [SMILES: CC(C)n1nc(-c2ccc3oc(N)nc3c2)c2c(N)ncnc21]

as described in Conejo-Garcia, J. R. et al., WO 2017/062426;

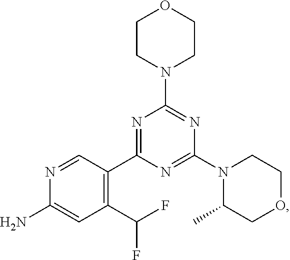 [SMILES: C[C@H]1COCCN1c1nc(-c2cnc(N)cc2C(F)F)nc(N2CCOCC2)n1]

as described in Rageot, D. et al., US2019/284178;

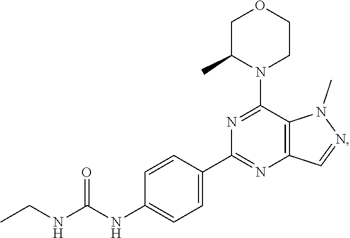 [SMILES: CCNC(=O)Nc1ccc(-c2nc(N3CCOC[C@@H]3C)c3c(cnn3C)n2)cc1]

as described in Pei, Z. et al., WO 2011/058025;

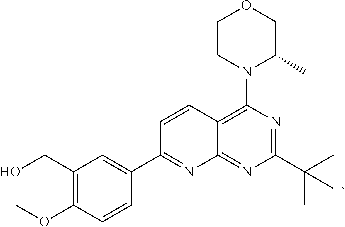 [SMILES: COc1ccc(-c2ccc3c(N4CCOC[C@@H]4C)nc(C(C)(C)C)nc3n2)cc1CO]

as described in Li, X. et al., WO 2013/016999;

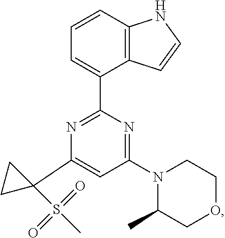 [SMILES: C[C@@H]1COCCN1c1cc(C2(S(C)(=O)=O)CC2)nc(-c2cccc3[nH]ccc23)n1]

as described in Foote, K. M. et al., WO 2010/073034;

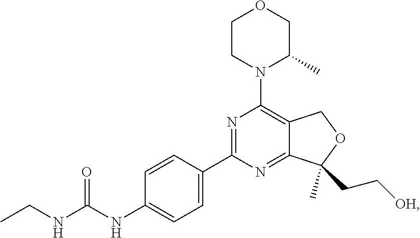 [SMILES: CCNC(=O)Nc1ccc(-c2nc(N3CCOC[C@@H]3C)c3c(n2)[C@](C)(CCO)OC3)cc1]

as described in Bergeron, P. et al., WO 2010/151601;

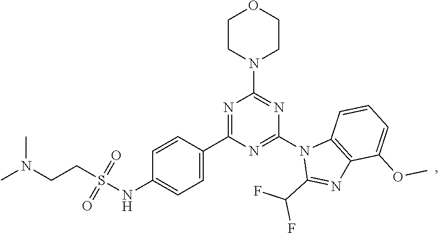

as described in Chen, L. et al., CN106806948.

In certain embodiments, the target protein ligand is an inhibitor and/or binder of alpha-synuclein. Inhibitors and/or binders of alpha-synuclein are reported in the literature. Exemplary inhibitors and/or binders of alpha-synuclein include the following compounds:

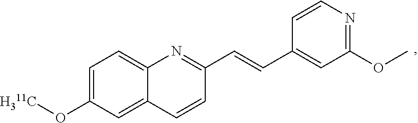

as described in Tu, Z. et al., US2017/189566;

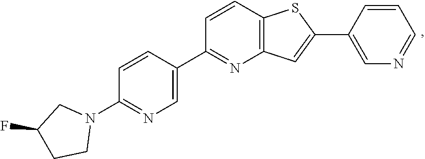 [SMILES: F[C@@H]1CCN(c2ccc(-c3ccc4sc(-c5cccnc5)cc4n3)cn2)C1]

as described in Molette, J. et al., WO 2017/153601;

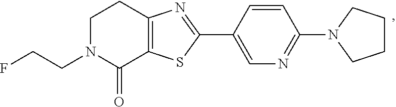 [SMILES: O=C1c2sc(-c3ccc(N4CCCC4)nc3)nc2CCN1CCF]

as described in Molette, J. et al., WO 2017/153601;

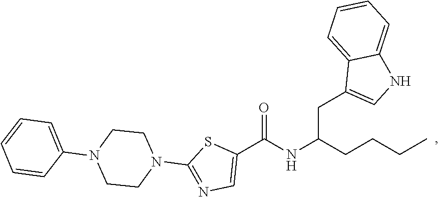 [SMILES: CCCCC(Cc1c[nH]c2ccccc12)NC(=O)c1cnc(N2CCN(c3ccccc3)CC2)s1]

as described in Hall, A. et al., WO 2018/138086.

In certain embodiments, the target protein ligand is an inhibitor and/or binder of human epidermal growth factor receptor 2 (HER2). Inhibitors and/or binders of HER2 are reported in the literature. Exemplary inhibitors and/or binders of HER2 include the following compounds:

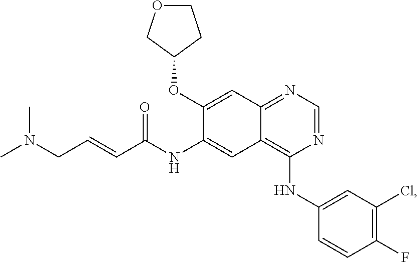 [SMILES: CN(C)C/C=C/C(=O)Nc1cc2c(Nc3ccc(F)c(Cl)c3)ncnc2cc1O[C@H]1CCOC1]

as described in Chen, J. et al., WO 2015/023703;

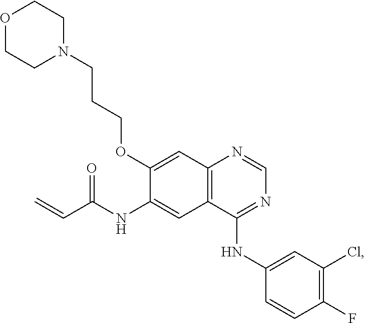 [SMILES: C=CC(=O)Nc1cc2c(Nc3ccc(F)c(Cl)c3)ncnc2cc1OCCCN1CCOCC1]

as described in Huang, Z. et al., WO 2012/027960;

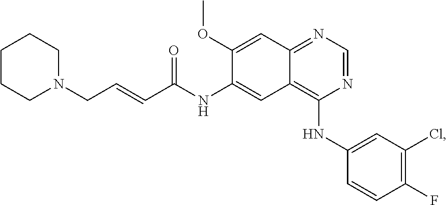 [SMILES: COc1cc2ncnc(Nc3ccc(F)c(Cl)c3)c2cc1NC(=O)/C=C/CN1CCCCC1]

as described in Wu, F. et al., WO 2012/159457;

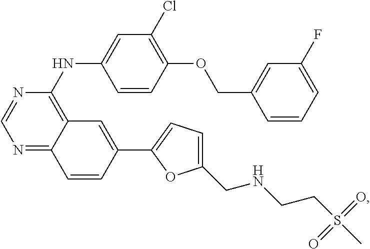 [SMILES: CS(=O)(=O)CCNCc1ccc(-c2ccc3ncnc(Nc4ccc(OCc5cccc(F)c5)c(Cl)c4)c3c2)o1]

as described in Wu, F. et al., WO 2012/159457;

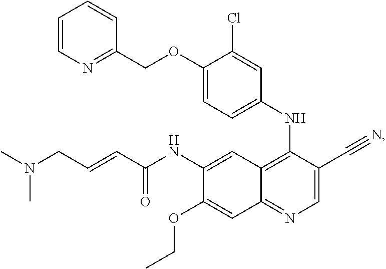 [SMILES: CCOc1cc2ncc(C#N)c(Nc3ccc(OCc4ccccn4)c(Cl)c3)c2cc1NC(=O)/C=C/CN(C)C]

as described in Wissner, A., et al., WO 2005/034955;

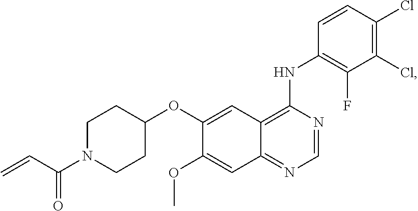 [SMILES: C=CC(=O)N1CCC(Oc2cc3c(Nc4ccc(Cl)c(Cl)c4F)ncnc3cc2OC)CC1]

as described in Li, Z. et al., WO 2019/149164;

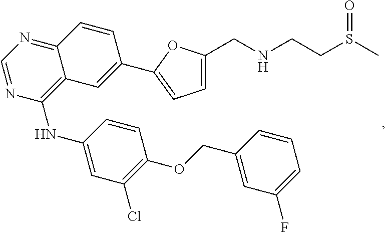 [SMILES: CS(=O)CCNCc1ccc(-c2ccc3ncnc(Nc4ccc(OCc5cccc(F)c5)c(Cl)c4)c3c2)o1]

as described in Wang, J. et al., WO 2011/035540;

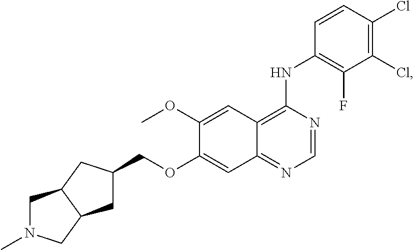 [SMILES: COc1cc2c(Nc3ccc(Cl)c(Cl)c3F)ncnc2cc1OC[C@@H]1C[C@@H]2CN(C)C[C@@H]2C1]

as described in Frost, P. et al., WO 2012/027537;

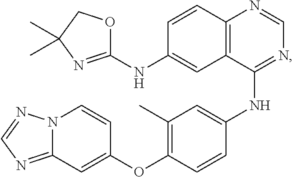 [SMILES: Cc1cc(Nc2ncnc3ccc(NC4=NC(C)(C)CO4)cc23)ccc1Oc1ccn2ncnc2c1]

as described in Xia, G. et al., WO 2017/148391;

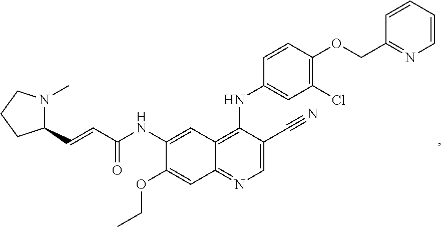 [SMILES: CCOc1cc2ncc(C#N)c(Nc3ccc(OCc4ccccn4)c(Cl)c3)c2cc1NC(=O)/C=C/[C@H]1CCCN1C]

as described in Li, X. et al., WO 2012/122865.

In certain embodiments, the target protein ligand is an inhibitor and/or binder of von Hippel-Lindau Disease Tumor Suppressor (VHL). Inhibitors and/or binders of VHL are reported in the literature. Exemplary inhibitors and/or binders of VHL include the following compounds:

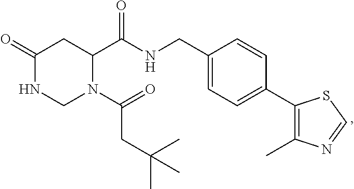 [SMILES: Cc1ncsc1-c1ccc(CNC(=O)C2CC(=O)NCN2C(=O)CC(C)(C)C)cc1]

as described in Xue, X., et al., CN109678852;

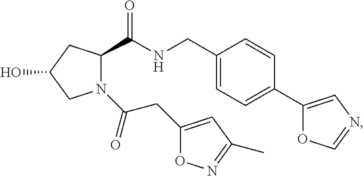 [SMILES: Cc1cc(CC(=O)N2C[C@H](O)C[C@H]2C(=O)NCc2ccc(-c3cnco3)cc2)on1]

as described in Crews, C. M. et al., WO 2013/106646;

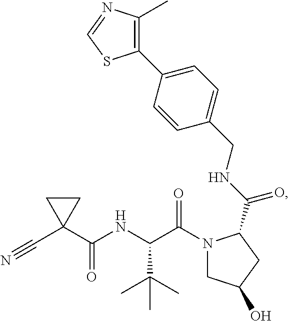 [SMILES: Cc1ncsc1-c1ccc(CNC(=O)[C@@H]2C[C@@H](O)CN2C(=O)[C@@H](NC(=O)C2(C#N)CC2)C(C)(C)C)cc1]

as described in Soares, P. et al., J Med Chem 2018, vol. 61(2), page 599.

In certain embodiments, the target protein ligand is an inhibitor and/or binder of Tyrosine-protein kinase ITK/TSK also known as interleukin-2-inducible T-cell kinase (ITK). Inhibitors and/or binders of ITK are reported in the literature. Exemplary inhibitors and/or binders of ITK include the following compounds:

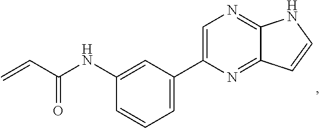 [SMILES: C=CC(=O)Nc1cccc(-c2cnc3[nH]ccc3n2)c1]

as described in Vankayalapati, H. et al., WO 2014/172513;

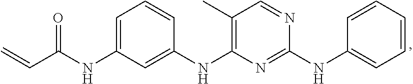 [SMILES: C=CC(=O)Nc1cccc(Nc2nc(Nc3ccccc3)ncc2C)c1]

as described in Kluge, A. F., et al., WO 2009/158571;

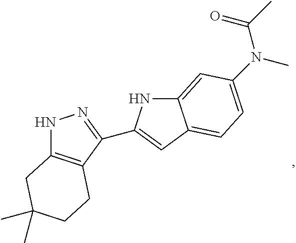

as described in Inoue, T., et al., WO 2011/065402;

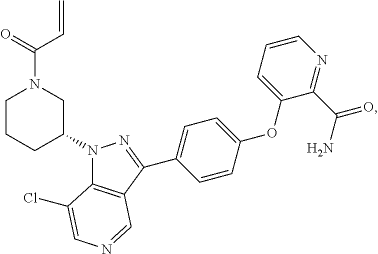 [SMILES: C=CC(=O)N1CCC[C@@H](n2nc(-c3ccc(Oc4cccnc4C(N)=O)cc3)c3cncc(Cl)c32)C1]

as described in Gavrilov, A. S., et al., WO 2018/092047;

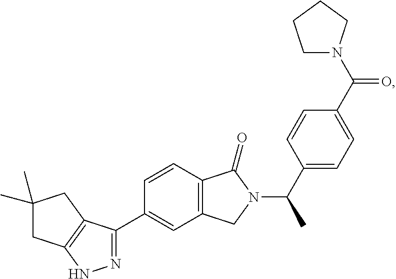 [SMILES: C[C@H](c1ccc(C(=O)N2CCCC2)cc1)N1Cc2cc(-c3n[nH]c4c3CC(C)(C)C4)ccc2C1=O]

as described in Tachibana, Y., et al., WO 2016/010108;

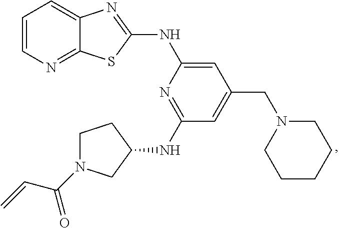 [SMILES: C=CC(=O)N1CC[C@H](Nc2cc(CN3CCCCC3)cc(Nc3nc4cccnc4s3)n2)C1]

as described in Barton, N. P., et al., WO 2012/035055;

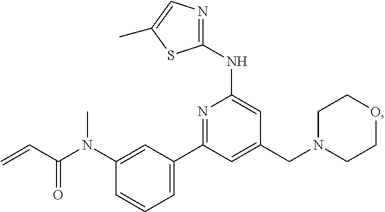 [SMILES: C=CC(=O)N(C)c1cccc(-c2cc(CN3CCOCC3)cc(Nc3ncc(C)s3)n2)c1]

as described in Kim, W. Y., et al., WO 2019/074275;

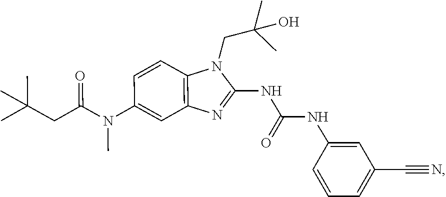 [SMILES: CN(C(=O)CC(C)(C)C)c1ccc2c(c1)nc(NC(=O)Nc1cccc(C#N)c1)n2CC(C)(C)O]

as described in Chaudhari, S. S. et. al., WO 2013/024427;

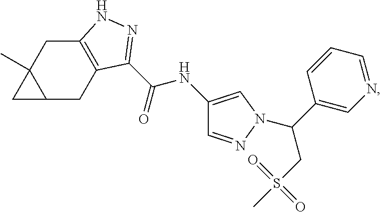 [SMILES: CC12Cc3[nH]nc(C(=O)Nc4cnn(C(CS(C)(=O)=O)c5cccnc5)c4)c3CC1C2]

as described in Brookfield, F., et al., 2014/023258;

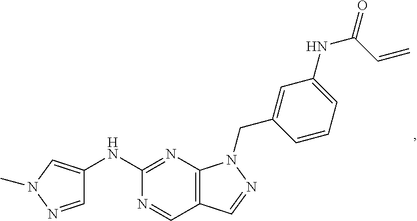 [SMILES: C=CC(=O)Nc1cccc(Cn2ncc3cnc(Nc4cnn(C)c4)nc32)c1]

as described in Ramsden, N., et al., WO 2013/041605;

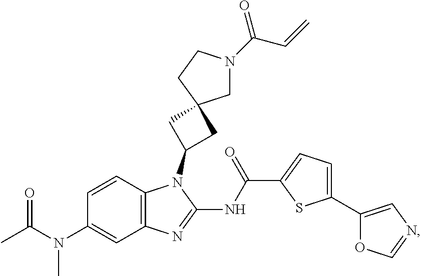 [SMILES: C=CC(=O)N1CC[C@]2(C1)C[C@H](n1c(NC(=O)c3ccc(-c4cnco4)s3)nc3cc(N(C)C(C)=O)ccc31)C2]

as described in Laurent, A., et al. WO 2018/032104;

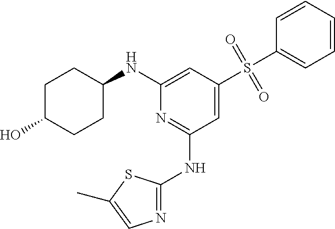 [SMILES: Cc1cnc(Nc2cc(S(=O)(=O)c3ccccc3)cc(N[C@H]3CC[C@H](O)CC3)n2)s1]

as described in Bromidge, S., et al. WO 2016/001341;

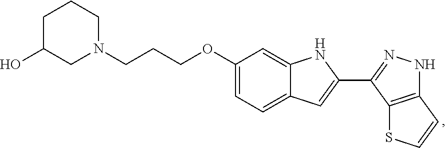 [SMILES: OC1CCCN(CCCOc2ccc3cc(-c4n[nH]c5ccsc45)[nH]c3c2)C1]

as described in Jurcak, J. G., et al., WO 2005/026175;

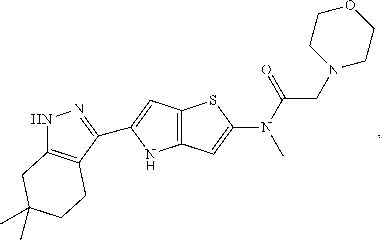 [SMILES: CN(C(=O)CN1CCOCC1)c1cc2[nH]c(-c3n[nH]c4c3CCC(C)(C)C4)cc2s1]

as described in Kumar, S., et al., WO 2014/041518;

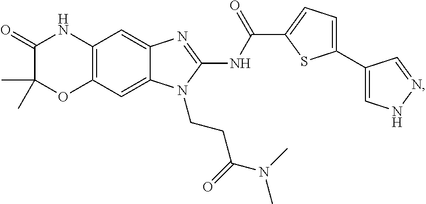 [SMILES: CN(C)C(=O)CCn1c(NC(=O)c2ccc(-c3cn[nH]c3)s2)nc2cc3c(cc21)OC(C)(C)C(=O)N3]

as described in Chaudhari, S. S., et al., WO 2013/153539.

In certain embodiments, the target protein ligand is an inhibitor and/or binder of phosphoinositide-dependent protein kinase-1 (PDK1). Inhibitors and/or binders of PDK1 are reported in the literature. Exemplary inhibitors and/or binders of PDK1 include the following compounds:

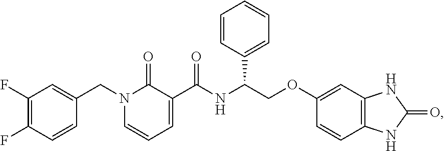 [SMILES: O=C(N[C@@H](COc1ccc2[nH]c(=O)[nH]c2c1)c1ccccc1)c1cccn(Cc2ccc(F)c(F)c2)c1=O]

as described in Erlanson, D. A. et al., Bioorg Med Chem Lett 2011, vol 21(10), page 3078;

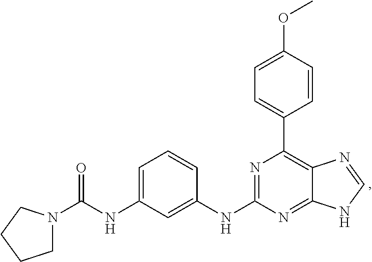 [SMILES: COc1ccc(-c2nc(Nc3cccc(NC(=O)N4CCCC4)c3)nc3[nH]cnc23)cc1]

as described in Blanchard, S. et al., Bioorg Med Chem Lett 2012, vol 22(8), page 2880;

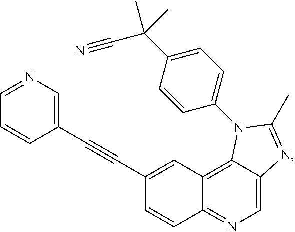 [SMILES: Cc1nc2cnc3ccc(C#Cc4cccnc4)cc3c2n1-c1ccc(C(C)(C)C#N)cc1]

as described in Stauffer, F. et al., Bioorg Med Chem Lett 2008, vol 18(3), page 1027;

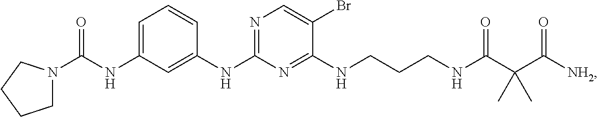 [SMILES: CC(C)(C(N)=O)C(=O)NCCCNc1nc(Nc2cccc(NC(=O)N3CCCC3)c2)ncc1Br]

as described in Feldman, R. I. et al., J Biol Chem 2005, vol 280(20), page 19867;

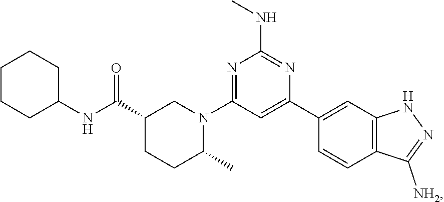 [SMILES: CNc1nc(-c2ccc3c(N)n[nH]c3c2)cc(N2C[C@@H](C(=O)NC3CCCCC3)CC[C@H]2C)n1]

as described in Axten, J. M. et al., WO 2010/059658;

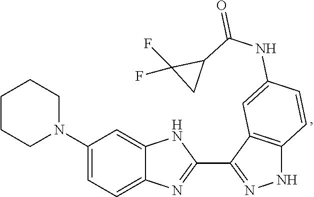 [SMILES: O=C(Nc1ccc2[nH]nc(-c3nc4ccc(N5CCCCC5)cc4[nH]3)c2c1)C1CC1(F)F]

as described in Chen, T. et al., W Bioorg Med Chem Lett 2017, vol 27(24), page 5473.

In certain embodiments, the target protein ligand is an inhibitor of epidermal growth factor receptor (EGFR). Inhibitors of EGFR are reported in the literature. Exemplary inhibitors of EGFR include the following compounds:

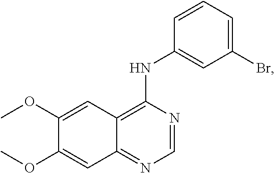 [SMILES: COc1cc2ncnc(Nc3cccc(Br)c3)c2cc1OC]

as described in Gangjee, A. et al., WO 2012/106522;

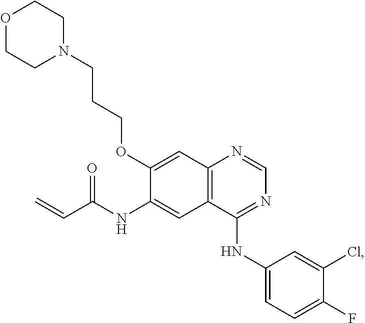 [SMILES: C=CC(=O)Nc1cc2c(Nc3ccc(F)c(Cl)c3)ncnc2cc1OCCCN1CCOCC1]

as described in Huang, Z. et al., WO 2012/027960;

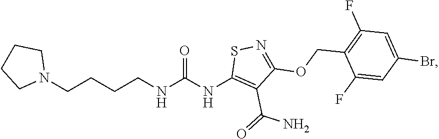 [SMILES: NC(=O)c1c(OCc2c(F)cc(Br)cc2F)nsc1NC(=O)NCCCCN1CCCC1]

as described in Bingaman, D. P. et al., WO 2014/152661;

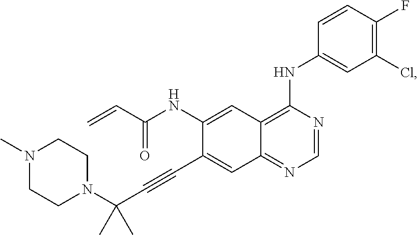 [SMILES: C=CC(=O)Nc1cc2c(Nc3ccc(F)c(Cl)c3)ncnc2cc1C#CC(C)(C)N1CCN(C)CC1]

as described in Kitano, Y. et al., WO 2002/066445;

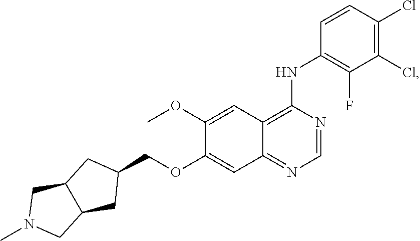 [SMILES: COc1cc2c(Nc3ccc(Cl)c(Cl)c3F)ncnc2cc1OC[C@@H]1C[C@@H]2CN(C)C[C@@H]2C1]

as described in Frost, P. et al., WO 2012/027537;

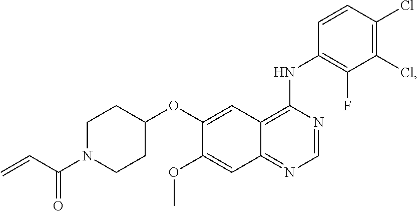 [SMILES: C=CC(=O)N1CCC(Oc2cc3c(Nc4ccc(Cl)c(Cl)c4F)ncnc3cc2OC)CC1]

as described in Lee, K.-O. et al., WO 2008/150118;

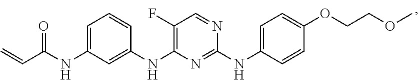 [SMILES: C=CC(=O)Nc1cccc(Nc2nc(Nc3ccc(OCCOC)cc3)ncc2F)c1]

as described in Kluge, A. F. et al., WO 2009/158571;

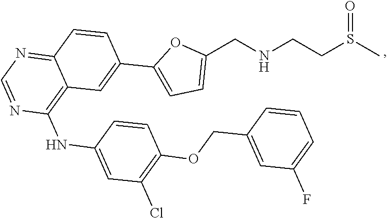 [SMILES: CS(=O)CCNCc1ccc(-c2ccc3ncnc(Nc4ccc(OCc5cccc(F)c5)c(Cl)c4)c3c2)o1]

as described in Wang, J. et al., WO 2011/035540;

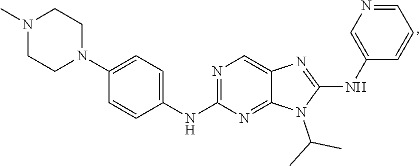 [SMILES: CC(C)n1c(Nc2cccnc2)nc2cnc(Nc3ccc(N4CCN(C)CC4)cc3)nc21]

as described in Yang, S. et al., WO 2011/147066;

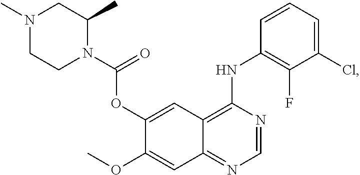 [SMILES: COc1cc2ncnc(Nc3cccc(Cl)c3F)c2cc1OC(=O)N1CCN(C)C[C@H]1C]

as described in Li, D. Y. et al., WO 2014/135876;

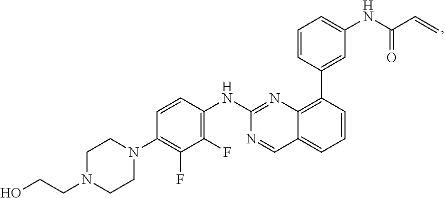 [SMILES: C=CC(=O)Nc1cccc(-c2cccc3cnc(Nc4ccc(N5CCN(CCO)CC5)c(F)c4F)nc23)c1]

as described in Qian, X. et. al., WO 2015/027222;

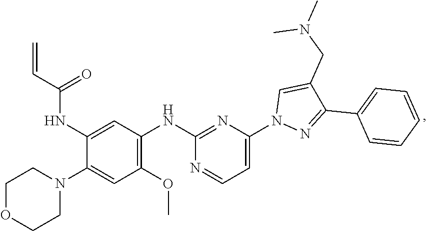 [SMILES: C=CC(=O)Nc1cc(Nc2nccc(-n3cc(CN(C)C)c(-c4ccccc4)n3)n2)c(OC)cc1N1CCOCC1]

as described in Suh, B.-C. et al., WO 2016/060443;

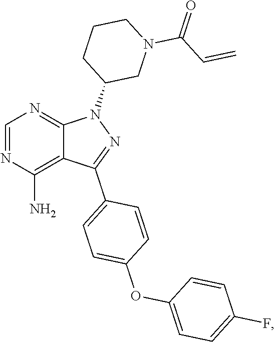 [SMILES: C=CC(=O)N1CCC[C@@H](n2nc(-c3ccc(Oc4ccc(F)cc4)cc3)c3c(N)ncnc32)C1]

as described in Zhang, D. et al., WO 2014/187319;

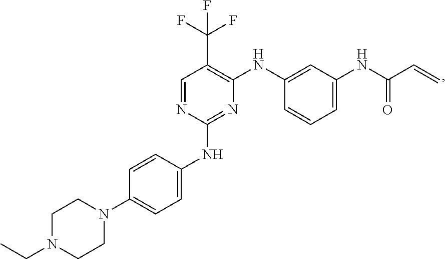 [SMILES: C=CC(=O)Nc1cccc(Nc2nc(Nc3ccc(N4CCN(CC)CC4)cc3)ncc2C(F)(F)F)c1]

as described in Zhang, D. et al., WO 2015/117547;

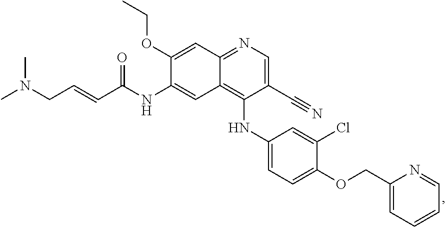 [SMILES: CCOc1cc2ncc(C#N)c(Nc3ccc(OCc4ccccn4)c(Cl)c3)c2cc1NC(=O)/C=C/CN(C)C]

as described in Zou, J. et al., WO 2018/054348;

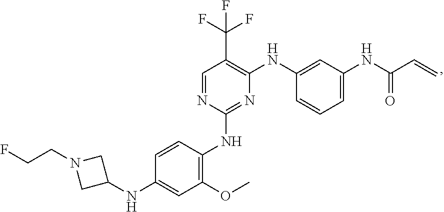 [SMILES: C=CC(=O)Nc1cccc(Nc2nc(Nc3ccc(NC4CN(CCF)C4)cc3OC)ncc2C(F)(F)F)c1]

as described in Lee, K. et al., WO 2012/064706.

In certain embodiments, the target protein ligand is an inhibitor of Mitogen-activated protein kinase kinase (MEK1/2). Inhibitors of MEK1/2 are reported in the literature. Exemplary inhibitors of ME1/2 include the following compounds:

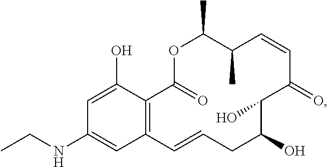 [SMILES: CCNc1cc(O)c2c(c1)/C=C/C[C@H](O)[C@H](O)C(=O)/C=C\[C@@H](C)[C@H](C)OC2=O]

as described in Narita, Y. et al., Mol Cancer Ther 2014, vol 13(4), page 823;

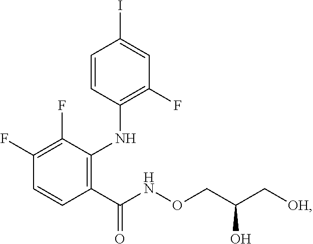 [SMILES: O=C(NOC[C@H](O)CO)c1ccc(F)c(F)c1Nc1ccc(I)cc1F]

as described in Berger, D. M. et al., Bioorg Med Chem 2008, vol 16(20), page 9202;

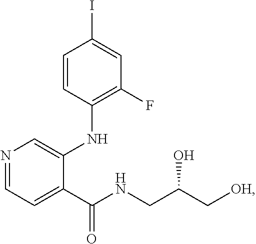 [SMILES: O=C(NC[C@H](O)CO)c1ccncc1Nc1ccc(I)cc1F]

as described in Haq, R. et al., WO 2014/138338;

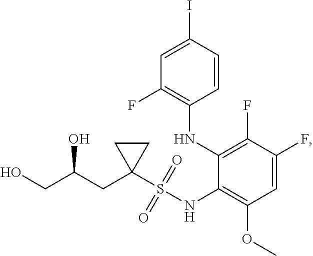 [SMILES: COc1cc(F)c(F)c(Nc2ccc(I)cc2F)c1NS(=O)(=O)C1(C[C@H](O)CO)CC1]

as described in Iverson, C. et al., Cancer Res 2009, vol 69(17), page 6839;

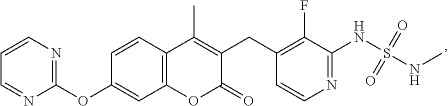 [SMILES: CNS(=O)(=O)Nc1nccc(Cc2c(C)c3ccc(Oc4ncccn4)cc3oc2=O)c1F]

as described in Aoki, T. et al., ACS Med Chem Lett 2014, vol 5(4), page 309;

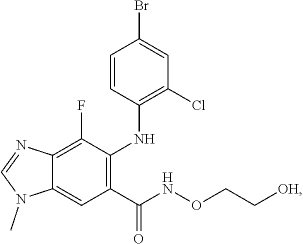 [SMILES: Cn1cnc2c(F)c(Nc3ccc(Br)cc3Cl)c(C(=O)NOCCO)cc21]

as described in Haq, R. et al., WO 2014/138338;

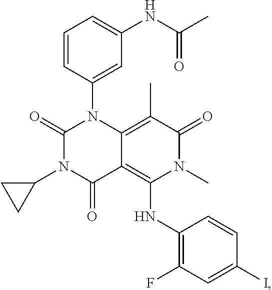 [SMILES: CC(=O)Nc1cccc(-n2c(=O)n(C3CC3)c(=O)c3c(Nc4ccc(I)cc4F)n(C)c(=O)c(C)c32)c1]

as described in Haq, R. et al., WO 2014/138338.

In certain embodiments, the target protein ligand is a binder and/or inhibitor of Fibroblast Growth Factor Receptor 1 (FGFR1). Binders and/or inhibitors of FGFR1 are reported in the literature. Exemplary binders and/or inhibitors of FGFR1 include the following compounds:

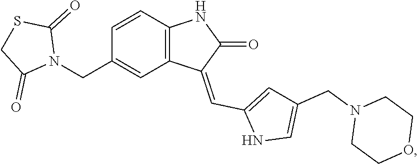 [SMILES: O=C1Nc2ccc(CN3C(=O)CSC3=O)cc2/C1=C/c1cc(CN2CCOCC2)c[nH]1]

as described in Burbridge, M. F. et al., Mol Cancer Ther 2013, vol. 12(9), page 1749;

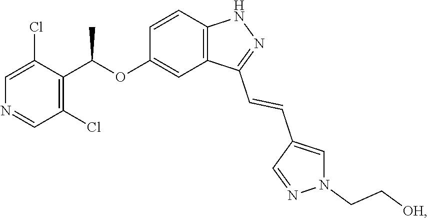 [SMILES: C[C@@H](Oc1ccc2[nH]nc(/C=C/c3cnn(CCO)c3)c2c1)c1c(Cl)cncc1Cl]

as described in Chen, D. et al., WO 2010/129509;

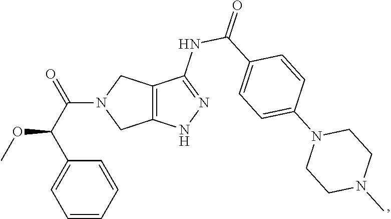

as described in Fancelli, D. et al., J Med Chem 2006, vol. 49(24), page 7247;

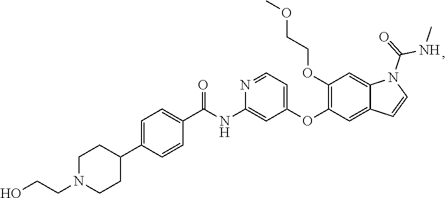 [SMILES: CNC(=O)n1ccc2cc(Oc3ccnc(NC(=O)c4ccc(C5CCN(CCO)CC5)cc4)c3)c(OCCOC)cc21]

as described in Funasaka, S. et al., WO 2014/129477;

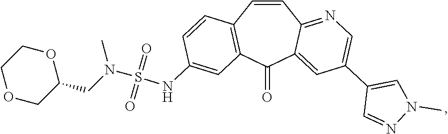 [SMILES: CN(C[C@@H]1COCCO1)S(=O)(=O)Nc1ccc2ccc3ncc(-c4cnn(C)c4)cc3c(=O)c2c1]

as described in Katz, J. D. et al., J Med Chem 2011, vol. 54(12), page 4092;

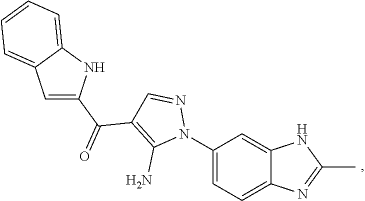 [SMILES: Cc1nc2ccc(-n3ncc(C(=O)c4cc5ccccc5[nH]4)c3N)cc2[nH]1]

as described in Nakanishi, Y. et al., Mol Cancer Ther 2014, vol. 13(11), page 2547

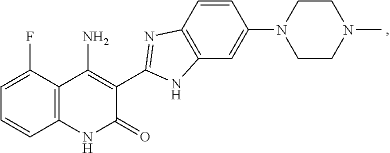 [SMILES: CN1CCN(c2ccc3nc(-c4c(N)c5c(F)cccc5[nH]c4=O)[nH]c3c2)CC1]

as described in Renhowe, P. A. et al., J Med Chem 2009, vol. 52(2), page 278;

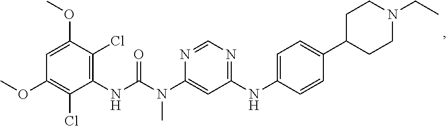 [SMILES: CCN1CCC(c2ccc(Nc3cc(N(C)C(=O)Nc4c(Cl)c(OC)cc(OC)c4Cl)ncn3)cc2)CC1]

as described in Reynolds, D. et al., WO 2015/057938;

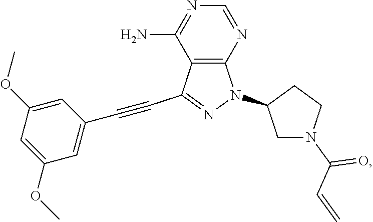 [SMILES: C=CC(=O)N1CC[C@H](n2nc(C#Cc3cc(OC)cc(OC)c3)c3c(N)ncnc32)C1]

as described in Sagara, T. et al., WO 2013/108809;

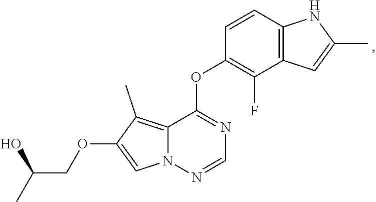 [SMILES: Cc1cc2c(F)c(Oc3ncnn4cc(OC[C@@H](C)O)c(C)c34)ccc2[nH]1]

as described in Squires, M. et al., Mol Cancer Ther 2011, vol. 10(9), page 1542;

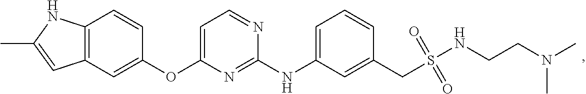

as described in Su, W.-G. et al., WO 2011/060746;

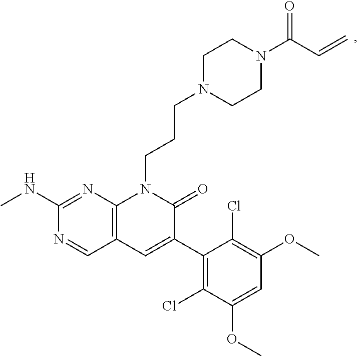 [SMILES: C=CC(=O)N1CCN(CCCn2c(=O)c(-c3c(Cl)c(OC)cc(OC)c3Cl)cc3cnc(NC)nc32)CC1]

as described in Venetsanakos, E., et al., 107th Annu Meet Am Assoc Cancer Res (AACR) (April 16-20, New Orleans) 2016, Abstract 1249;

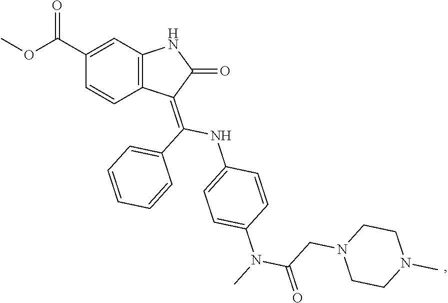

as described in Walters, I. et al., WO 2017/109513;

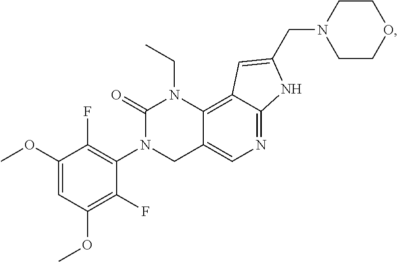 [SMILES: CCN1C(=O)N(c2c(F)c(OC)cc(OC)c2F)Cc2cnc3[nH]c(CN4CCOCC4)cc3c21]

as described in Wu, L. et al., WO 2014/007951;

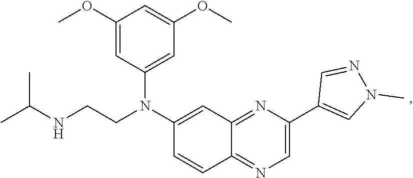 [SMILES: COc1cc(OC)cc(N(CCNC(C)C)c2ccc3ncc(-c4cnn(C)c4)nc3c2)c1]

as described in Xu, X. et al., WO 2018/153373;

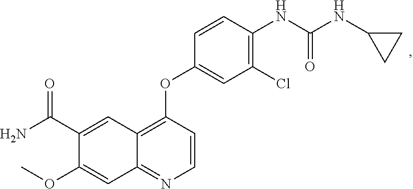 [SMILES: COc1cc2nccc(Oc3ccc(NC(=O)NC4CC4)c(Cl)c3)c2cc1C(N)=O]

as described in Zhang, Y. et al., WO 2019/062637.

In certain embodiments, the target protein ligand is a binder and/or inhibitor of Fibroblast Growth Factor Receptor 2 (FGFR2). Binders and/or inhibitors of FGFR2 are reported in the literature. Exemplary binders and/or inhibitors of FGFR2 include the following compounds:

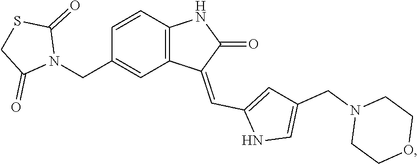 [SMILES: O=C1Nc2ccc(CN3C(=O)CSC3=O)cc2/C1=C/c1cc(CN2CCOCC2)c[nH]1]

as described in Burbridge, M. F. et al., Mol Cancer Ther 2013, vol. 12(9), page 1749;

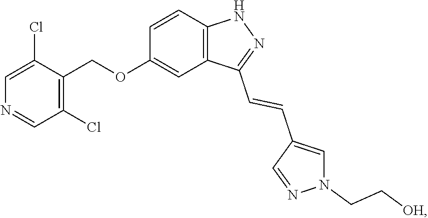 [SMILES: OCCn1cc(/C=C/c2n[nH]c3ccc(OCc4c(Cl)cncc4Cl)cc23)cn1]

as described in Chen, D. et al., WO 2010/129509;

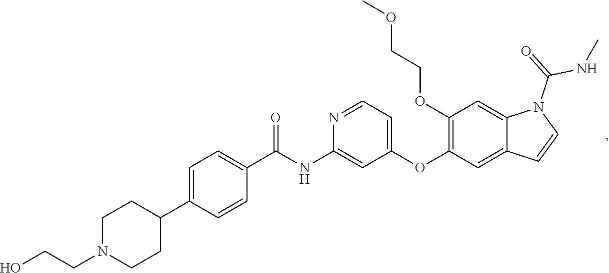 [SMILES: CNC(=O)n1ccc2cc(Oc3ccnc(NC(=O)c4ccc(C5CCN(CCO)CC5)cc4)c3)c(OCCOC)cc21]

as described in Funasaka, S. et al., WO 201, vol. 54(129477;

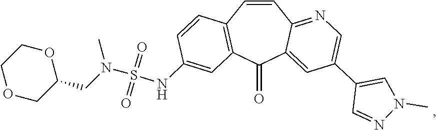 [SMILES: CN(C[C@@H]1COCCO1)S(=O)(=O)Nc1ccc2ccc3ncc(-c4cnn(C)c4)cc3c(=O)c2c1]

as described in Katz, J. D. et al., J Med Chem 2011, vol. 54(12), page 4092;

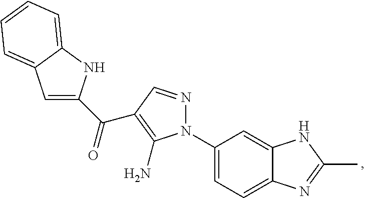 [SMILES: Cc1nc2ccc(-n3ncc(C(=O)c4cc5ccccc5[nH]4)c3N)cc2[nH]1]

as described in Nakanishi, Y. et al., Mol Cancer Ther 2014, vol. 13(11), page 2547;

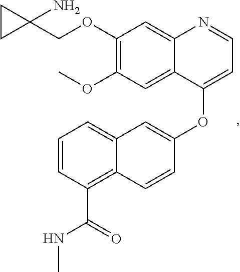 [SMILES: CNC(=O)c1cccc2cc(Oc3ccnc4cc(OCC5(N)CC5)c(OC)cc34)ccc12]

as described in Naguyen, M., et al., 106th Annu Meet Am Assoc Cancer Res (AACR) (April 18-22, Philadelphia) 2015, Abstract 784;

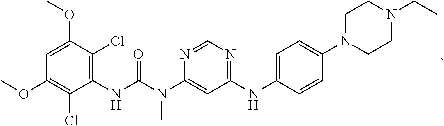 [SMILES: CCN1CCN(c2ccc(Nc3cc(N(C)C(=O)Nc4c(Cl)c(OC)cc(OC)c4Cl)ncn3)cc2)CC1]

as described in Reynolds, D. et al., WO 2015/057938;

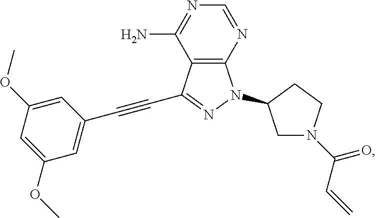 [SMILES: C=CC(=O)N1CC[C@H](n2nc(C#Cc3cc(OC)cc(OC)c3)c3c(N)ncnc32)C1]

as described in Sagara, T. et al., WO 2013/108809;

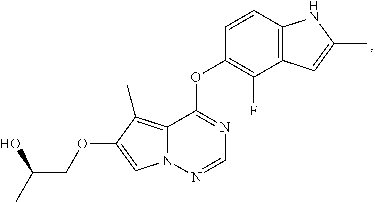 [SMILES: Cc1cc2c(F)c(Oc3ncnn4cc(OC[C@@H](C)O)c(C)c34)ccc2[nH]1]

as described in Squires, M. et al., Mol Cancer Ther 2011, vol. 10(9), page 1542;

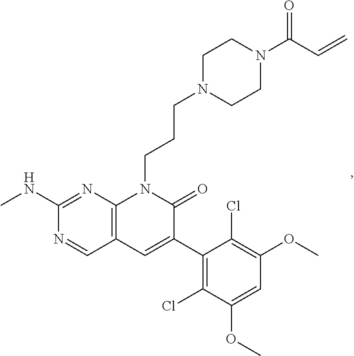 [SMILES: C=CC(=O)N1CCN(CCCn2c(=O)c(-c3c(Cl)c(OC)cc(OC)c3Cl)cc3cnc(NC)nc32)CC1]

as described in Venetsanakos, E., et al., 107th Annu Meet Am Assoc Cancer Res (AACR) (April 16-20, New Orleans) 2016, Abstract 1249;

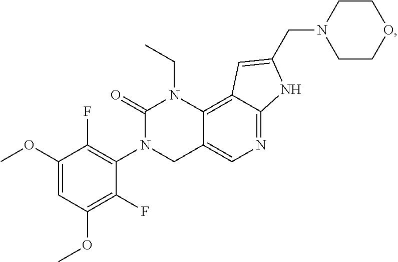 [SMILES: CCN1C(=O)N(c2c(F)c(OC)cc(OC)c2F)Cc2cnc3[nH]c(CN4CCOCC4)cc3c21]

as described in Wu, L. et al., WO 2014/007951;

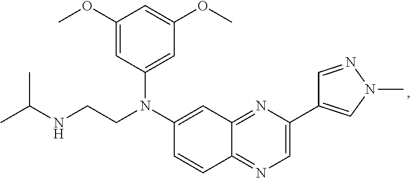 [SMILES: COc1cc(OC)cc(N(CCNC(C)C)c2ccc3ncc(-c4cnn(C)c4)nc3c2)c1]

as described in Xu, X. et al., WO 2018/153373.

In certain embodiments, the target protein ligand is a binder and/or inhibitor of Fibroblast Growth Factor Receptor 3 (FGFR3). Binders and/or inhibitors of FGFR3 are reported in the literature. Exemplary binders and/or inhibitors of FGFR3 include the following compounds:

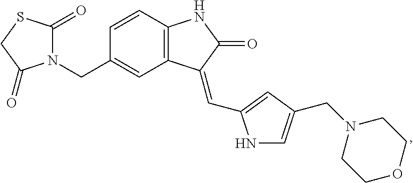 [SMILES: O=C1Nc2ccc(CN3C(=O)CSC3=O)cc2/C1=C/c1cc(CN2CCOCC2)c[nH]1]

as described in Burbridge, M. F. et al., Mol Cancer Ther 2013, vol. 12(9), page 1749;

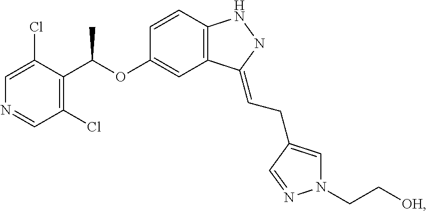 [SMILES: C[C@@H](Oc1ccc2c(c1)/C(=C/Cc1cnn(CCO)c1)NN2)c1c(Cl)cncc1Cl]

as described in Chen, D. et al., WO 2010/129509;

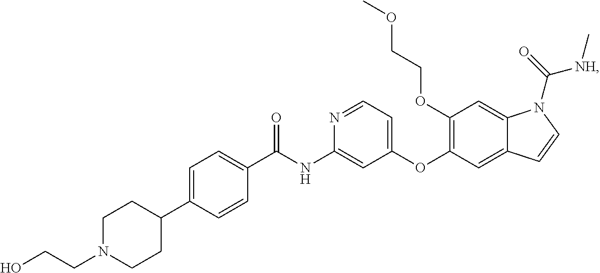 [SMILES: CNC(=O)n1ccc2cc(Oc3ccnc(NC(=O)c4ccc(C5CCN(CCO)CC5)cc4)c3)c(OCCOC)cc21]

as described in Funasaka, S. et al., WO 2014/129477:

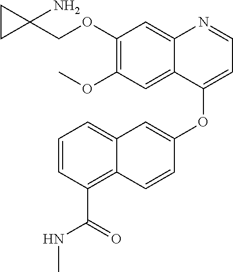 [SMILES: CNC(=O)c1cccc2cc(Oc3ccnc4cc(OCC5(N)CC5)c(OC)cc34)ccc12]

as described in Holmstroem, T. H., et al., Mol Cancer Ther 2019, vol. 18(1), page 28;

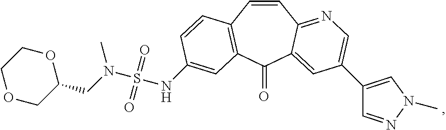 [SMILES: CN(C[C@@H]1COCCO1)S(=O)(=O)Nc1ccc2ccc3ncc(-c4cnn(C)c4)cc3c(=O)c2c1]

as described in Katz, J. D. et al., J Med Chem 2011, vol. 54(12), page 4092;

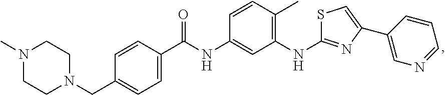 [SMILES: Cc1ccc(NC(=O)c2ccc(CN3CCN(C)CC3)cc2)cc1Nc1nc(-c2cccnc2)cs1]

as described in Moussy, A. et al., WO 2015/082496;

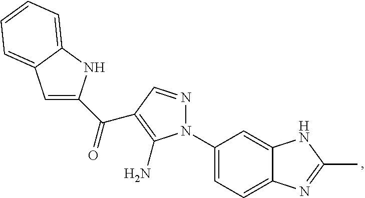 [SMILES: Cc1nc2ccc(-n3ncc(C(=O)c4cc5ccccc5[nH]4)c3N)cc2[nH]1]

as described in Nakanishi, Y. et al., Mol Cancer Ther 2014, vol. 13(11), page 2547;

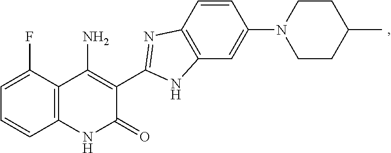 [SMILES: CC1CCN(c2ccc3nc(-c4c(N)c5c(F)cccc5[nH]c4=O)[nH]c3c2)CC1]

as described in Renhowe, P. A. et al., J Med Chem 2009, vol. 52(2), page 278;

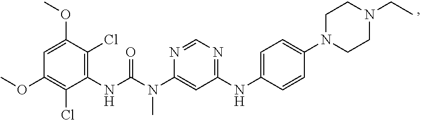 [SMILES: CCN1CCN(c2ccc(Nc3cc(N(C)C(=O)Nc4c(Cl)c(OC)cc(OC)c4Cl)ncn3)cc2)CC1]

as described in Reynolds, D. et al., WO 2015/057938;

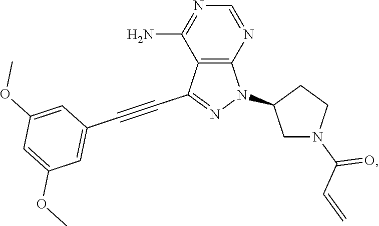 [SMILES: C=CC(=O)N1CC[C@H](n2nc(C#Cc3cc(OC)cc(OC)c3)c3c(N)ncnc32)C1]

as described in Sagara, T. et al., WO 2013/108809;

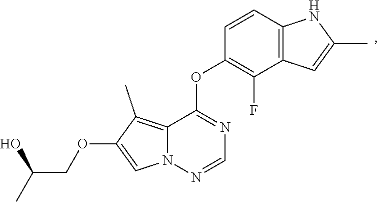 [SMILES: Cc1cc2c(F)c(Oc3ncnn4cc(OC[C@@H](C)O)c(C)c34)ccc2[nH]1]

as described in Squires, M. et al., Mol Cancer Ther 2011, vol. 10(9), page 1542;

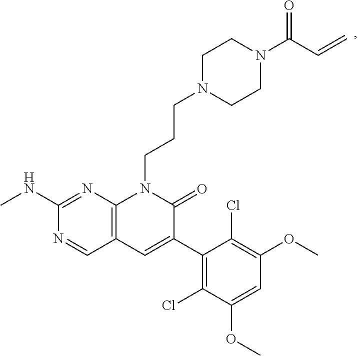 [SMILES: C=CC(=O)N1CCN(CCCn2c(=O)c(-c3c(Cl)c(OC)cc(OC)c3Cl)cc3cnc(NC)nc32)CC1]

as described in Venetsanakos, E., et al., 107th Annu Meet Am Assoc Cancer Res (AACR) (April 16-20, New Orleans) 2016, Abstract 1249;

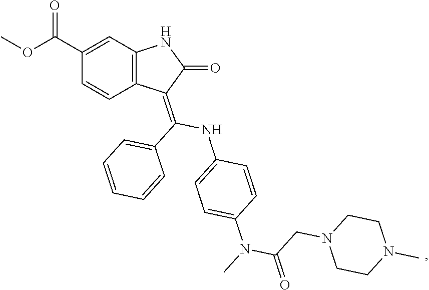

as described in Walters, I. et al., WO 2017/109513;

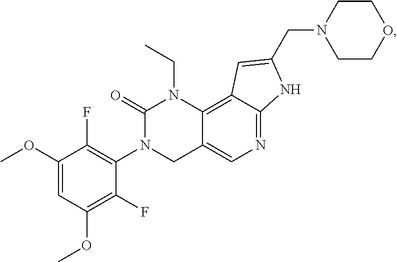 [SMILES: CCN1C(=O)N(c2c(F)c(OC)cc(OC)c2F)Cc2cnc3[nH]c(CN4CCOCC4)cc3c21]

as described in Wu, L. et al., WO 2014/007951.

In certain embodiments, the target protein ligand is a binder and/or inhibitor of Fibroblast Growth Factor Receptor 4 (FGFR4). Binders and/or inhibitors of FGFR4 are reported in the literature. Exemplary binders and/or inhibitors of FGFR4 include the following compounds:

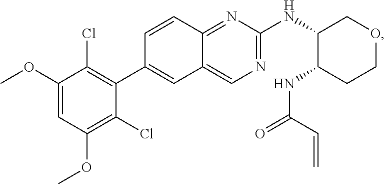 [SMILES: C=CC(=O)N[C@H]1CCOC[C@H]1Nc1ncc2cc(-c3c(Cl)c(OC)cc(OC)c3Cl)ccc2n1]

as described in Bifulco, N. Jr. et al., US2017/174652;

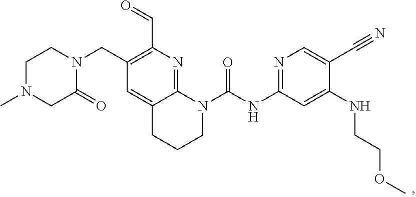

as described in Buschmann, N. et al., WO 2015/059668;

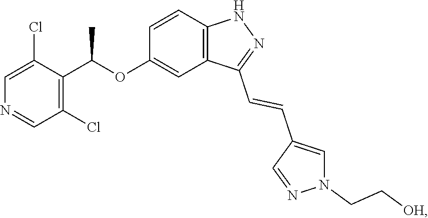 [SMILES: C[C@@H](Oc1ccc2[nH]nc(/C=C/c3cnn(CCO)c3)c2c1)c1c(Cl)cncc1Cl]

as described in Chen, D. et al., WO 2010/129509;

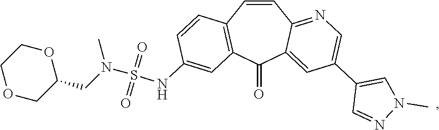 [SMILES: CN(C[C@@H]1COCCO1)S(=O)(=O)Nc1ccc2ccc3ncc(-c4cnn(C)c4)cc3c(=O)c2c1]

as described in Katz, J. D. et al., J Med Chem 2011, vol. 54(12), page 4092;

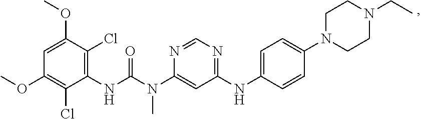 [SMILES: CCN1CCN(c2ccc(Nc3cc(N(C)C(=O)Nc4c(Cl)c(OC)cc(OC)c4Cl)ncn3)cc2)CC1]

as described in Reynolds, D. et al., WO 2015/057938;

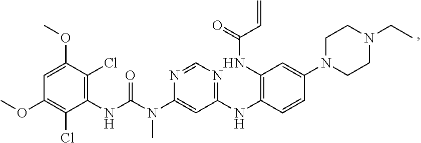 [SMILES: C=CC(=O)Nc1cc(N2CCN(CC)CC2)ccc1Nc1cc(N(C)C(=O)Nc2c(Cl)c(OC)cc(OC)c2Cl)ncn1]

as described in Reynolds, D. et al., WO 2015/057938;

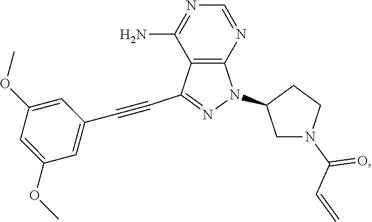 [SMILES: C=CC(=O)N1CC[C@H](n2nc(C#Cc3cc(OC)cc(OC)c3)c3c(N)ncnc32)C1]

as described in Sagara, T. et al., WO 2013/108809;

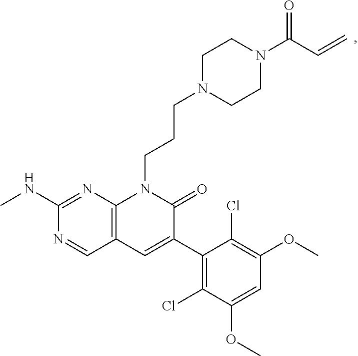 [SMILES: C=CC(=O)N1CCN(CCCn2c(=O)c(-c3c(Cl)c(OC)cc(OC)c3Cl)cc3cnc(NC)nc32)CC1]

as described in Venetsanakos, E., et al., 107th Annu Meet Am Assoc Cancer Res (AACR) (April 16-20, New Orleans) 2016, Abstract 1249;

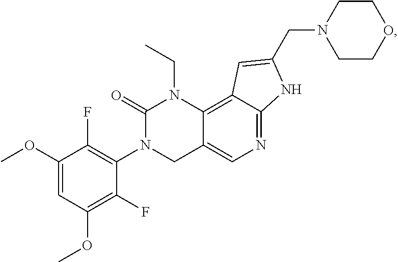 [SMILES: CCN1C(=O)N(c2c(F)c(OC)cc(OC)c2F)Cc2cnc3[nH]c(CN4CCOCC4)cc3c21]

as described in Wu, L. et al., WO 2014/007951;

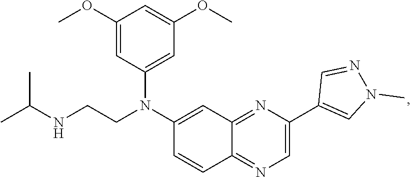 [SMILES: COc1cc(OC)cc(N(CCNC(C)C)c2ccc3ncc(-c4cnn(C)c4)nc3c2)c1]

as described in Xu, X. et al., WO 2018/153373.

In certain embodiments, the target protein ligand is an inhibitor of extracellular signal-regulated kinase 1 (ERK-1). Inhibitors of ERK-1 are reported in the literature. Exemplary inhibitors of ERK-1 include the following compounds:

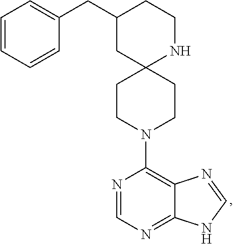 [SMILES: c1ccc(CC2CCNC3(CCN(c4ncnc5[nH]cnc45)CC3)C2)cc1]

as described in Allen, C. E. et al., in Bioorg Med Chem 2013, vol 21(18), page 5707;

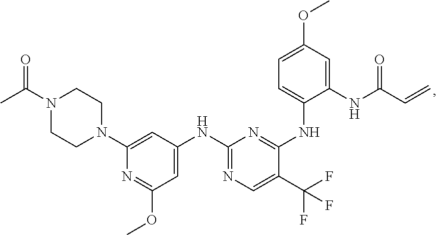 [SMILES: C=CC(=O)Nc1cc(OC)ccc1Nc1nc(Nc2cc(OC)nc(N3CCN(C(C)=O)CC3)c2)ncc1C(F)(F)F]

as described in Haq, N. et al., in WO 2014/124230;

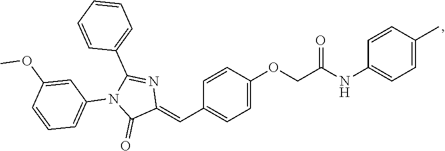

as described in Awadallah, F. M. et al., in Eur J Med Chem 2015, vol 94, page 397;

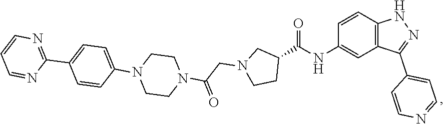 [SMILES: O=C(Nc1ccc2[nH]nc(-c3ccncc3)c2c1)[C@@H]1CCN(CC(=O)N2CCN(c3ccc(-c4ncccn4)cc3)CC2)C1]

as described in Huang, P. Q. et al., in WO 2016/161160;

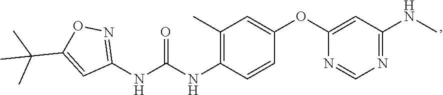 [SMILES: CNc1cc(Oc2ccc(NC(=O)Nc3cc(C(C)(C)C)on3)c(C)c2)ncn1]

as described in Chen, Y. et al., in Eur J Med Chem 2017, vol 127, page 997;

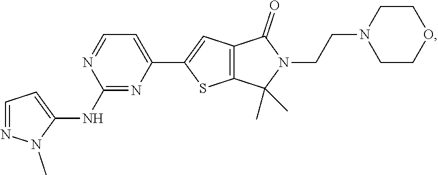 [SMILES: Cn1nccc1Nc1nccc(-c2cc3c(s2)C(C)(C)N(CCN2CCOCC2)C3=O)n1]

as described in Cortez, G. S. et al., in WO 2016/106029;

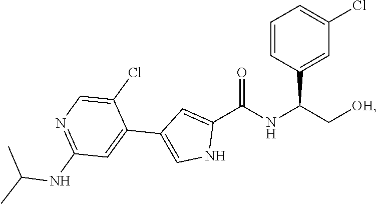 [SMILES: CC(C)Nc1cc(-c2c[nH]c(C(=O)N[C@H](CO)c3cccc(Cl)c3)c2)c(Cl)cn1]

as described in Huang, P. Q. et al., in WO 2016/161160;

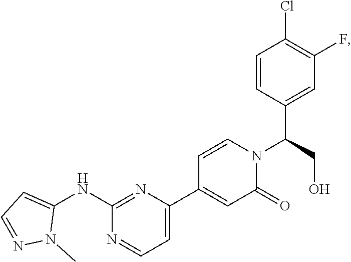 [SMILES: Cn1nccc1Nc1nccc(-c2ccn([C@H](CO)c3ccc(Cl)c(F)c3)c(=O)c2)n1]

as described in Huang, P. Q. et al., in WO 2016/161160;

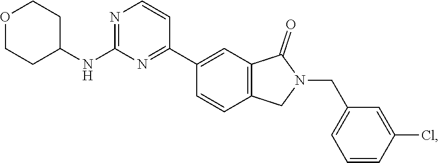 [SMILES: O=C1c2cc(-c3ccnc(NC4CCOCC4)n3)ccc2CN1Cc1cccc(Cl)c1]

as described in Ji, D. Z. et al., in Eur J Med Chem 2019, vol 164, page 334;

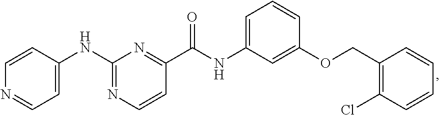 [SMILES: O=C(Nc1cccc(OCc2ccccc2Cl)c1)c1ccnc(Nc2ccncc2)n1]

as described in Kim, E. E. K. et al., in KR2012/092768;

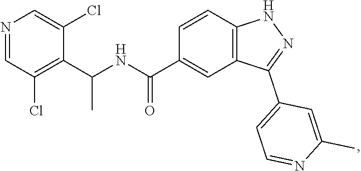 [SMILES: Cc1cc(-c2n[nH]c3ccc(C(=O)NC(C)c4c(Cl)cncc4Cl)cc23)ccn1]

as described in Li, L. et al., in Bioorg Med Chem Lett 2016, vol 26(11), page 2600;

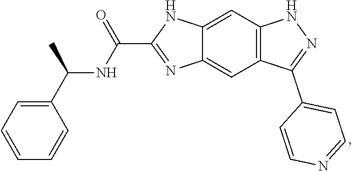 [SMILES: C[C@@H](NC(=O)c1nc2cc3c(-c4ccncc4)n[nH]c3cc2[nH]1)c1ccccc1]

as described in Liu, S. et al., in WO 2019/076336;

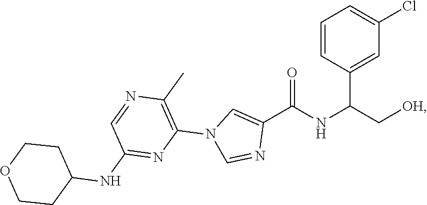 [SMILES: Cc1ncc(NC2CCOCC2)nc1-n1cnc(C(=O)NC(CO)c2cccc(Cl)c2)c1]

as described in Venkatesan, A. M. et al., in U.S. Pat. No. 9,896,445;

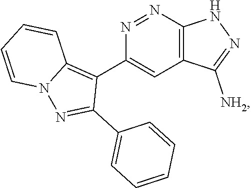 [SMILES: Nc1n[nH]c2nnc(-c3c(-c4ccccc4)nn4ccccc34)cc12]

as described in Zhang, C. et al., in J Pharmacol Exp Ther 2019, vol 370(2), page 206.

In certain embodiments, the target protein ligand is an inhibitor of extracellular signal-regulated kinase 2 (ERK-2). Inhibitors of ERK-2 are reported in the literature. Exemplary inhibitors of ERK-2 include the following compounds:

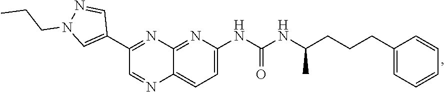 [SMILES: CCCn1cc(-c2cnc3ccc(NC(=O)N[C@H](C)CCCc4ccccc4)nc3n2)cn1]

as described in Gerlach, M. et al., in WO 2012/136691;

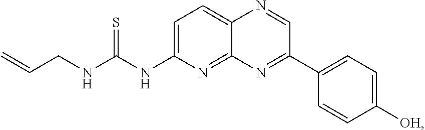 [SMILES: C=CCNC(=S)Nc1ccc2ncc(-c3ccc(O)cc3)nc2n1]

as described in Guenther, E. et al., in WO 2004/104002;

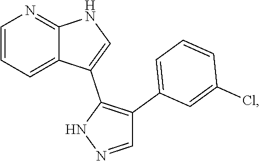 [SMILES: Clc1cccc(-c2cn[nH]c2-c2c[nH]c3ncccc23)c1]

as described in Fairfax, D. et al., in WO 2012/094313;

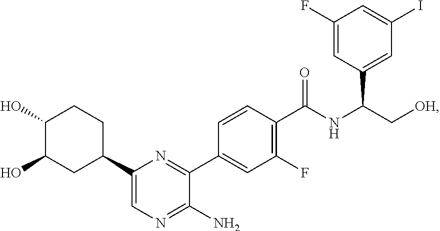 [SMILES: Nc1ncc([C@@H]2CC[C@@H](O)[C@H](O)C2)nc1-c1ccc(C(=O)N[C@H](CO)c2cc(F)cc(I)c2)c(F)c1]

as described in Bagdanoff, J. T. et al., in WO

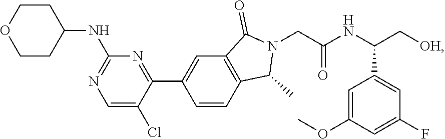 [SMILES: COc1cc(F)cc([C@@H](CO)NC(=O)CN2C(=O)c3cc(-c4nc(NC5CCOCC5)ncc4Cl)ccc3[C@H]2C)c1]

as described in Berdini, V. et al., in WO 2017/068412;

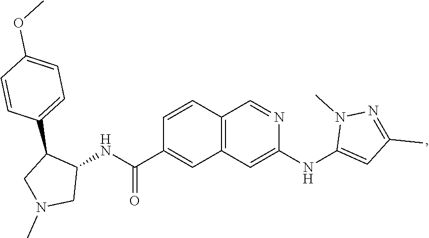 [SMILES: COc1ccc([C@@H]2CN(C)C[C@H]2NC(=O)c2ccc3cnc(Nc4cc(C)nn4C)cc3c2)cc1]

as described in Blake, J. et al., in WO 2014/036015;

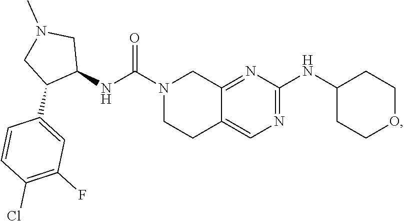 [SMILES: CN1C[C@@H](NC(=O)N2CCc3cnc(NC4CCOCC4)nc3C2)[C@H](c2ccc(Cl)c(F)c2)C1]

as described in Blake, J. F. et al., in WO 2012/118850;

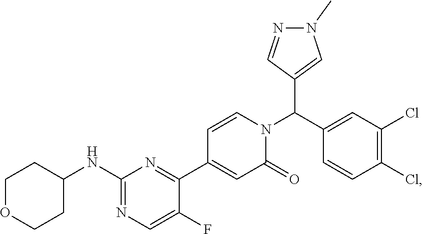 [SMILES: Cn1cc(C(c2ccc(Cl)c(Cl)c2)n2ccc(-c3nc(NC4CCOCC4)ncc3F)cc2=O)cn1]

as described in Blake, J. F. et al., in WO 2013/130976;

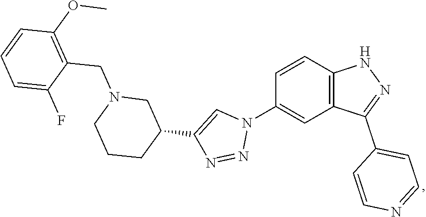 [SMILES: COc1cccc(F)c1CN1CCC[C@@H](c2cn(-c3ccc4[nH]nc(-c5ccncc5)c4c3)nn2)C1]

as described in Boga, S. B. et al., in WO 2012/058127;

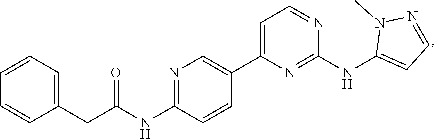 [SMILES: Cn1nccc1Nc1nccc(-c2ccc(NC(=O)Cc3ccccc3)nc2)n1]

as described in Cao, J. et al., in WO 2017/114510;

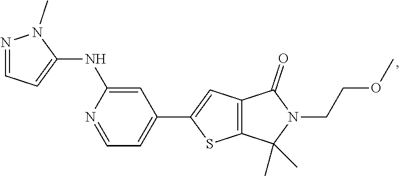 [SMILES: COCCN1C(=O)c2cc(-c3ccnc(Nc4ccnn4C)c3)sc2C1(C)C]

as described in Cortez, G. S. et al., in WO 2016/106009;

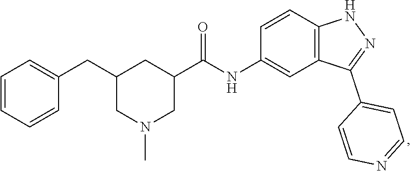 [SMILES: CN1CC(Cc2ccccc2)CC(C(=O)Nc2ccc3[nH]nc(-c4ccncc4)c3c2)C1]

as described in Deng, Y. et al., in WO 2012/030685;

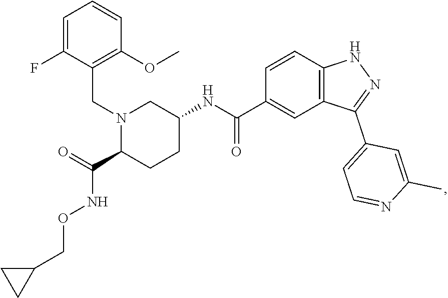 [SMILES: COc1cccc(F)c1CN1C[C@H](NC(=O)c2ccc3[nH]nc(-c4ccnc(C)c4)c3c2)CC[C@H]1C(=O)NOCC1CC1]

as described in Deng, Y. et al., in WO 2011/163330;

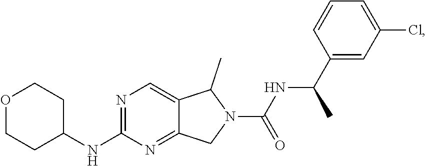 [SMILES: CC1c2cnc(NC3CCOCC3)nc2CN1C(=O)N[C@H](C)c1cccc(Cl)c1]

as described in Dillon, M. P. et al., in WO 2014/047020;

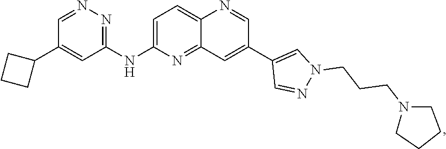 [SMILES: c1nc2ccc(Nc3cc(C4CCC4)cnn3)nc2cc1-c1cnn(CCCN2CCCC2)c1]

as described in Furuyama, H. et al., in WO 2014/109414;

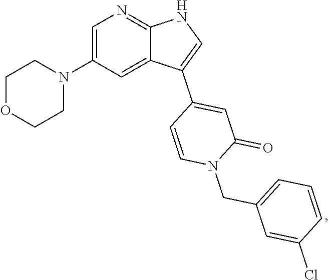 [SMILES: O=c1cc(-c2c[nH]c3ncc(N4CCOCC4)cc23)ccn1Cc1cccc(Cl)c1]

as described in Guichou, J.-F. et al., in WO 2017/085230;

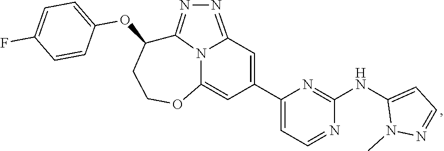 [SMILES: Cn1nccc1Nc1nccc(-c2cc3n4c(nnc4c2)[C@H](Oc2ccc(F)cc2)CCO3)n1]

as described in Kolesnikov, A. et al., in WO 2015/085007;

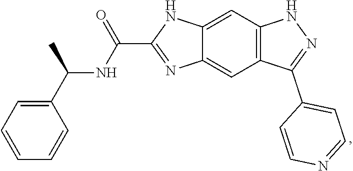 [SMILES: C[C@@H](NC(=O)c1nc2cc3c(-c4ccncc4)n[nH]c3cc2[nH]1)c1ccccc1]

as described in Liu, S. et al., in WO 2019/076336;

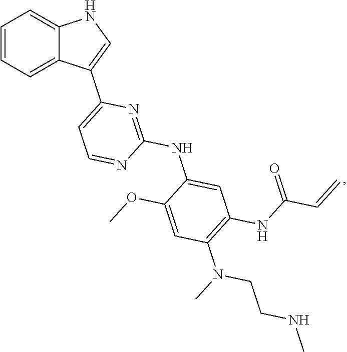 [SMILES: C=CC(=O)Nc1cc(Nc2nccc(-c3c[nH]c4ccccc34)n2)c(OC)cc1N(C)CCNC]

as described in Tang, J. et al., in CN107973783;

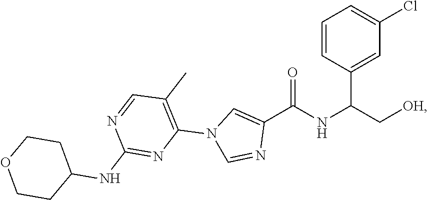 [SMILES: Cc1cnc(NC2CCOCC2)nc1-n1cnc(C(=O)NC(CO)c2cccc(Cl)c2)c1]

as described in Venkatesan, A. M. et al., in US 2016/362406;

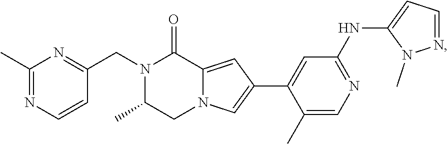 [SMILES: Cc1nccc(CN2C(=O)c3cc(-c4cc(Nc5ccnn5C)ncc4C)cn3C[C@@H]2C)n1]

as described in Ward, R. A. et al., in WO 2017/080980;

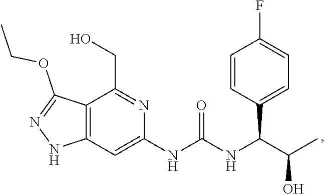 [SMILES: CCOc1n[nH]c2cc(NC(=O)N[C@@H](c3ccc(F)cc3)[C@@H](C)O)nc(CO)c12]

as described in Wilson, K. J. et al., in WO 2014/052566;

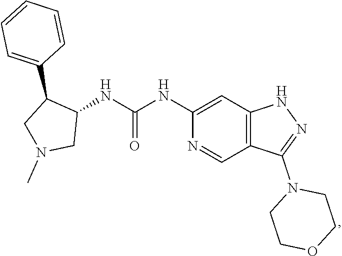 [SMILES: CN1C[C@@H](NC(=O)Nc2cc3[nH]nc(N4CCOCC4)c3cn2)[C@H](c2ccccc2)C1]

as described in Wilson, K. J. et al., in WO 2014/052563;

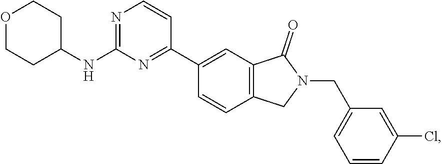 [SMILES: O=C1c2cc(-c3ccnc(NC4CCOCC4)n3)ccc2CN1Cc1cccc(Cl)c1]

as described in Xu, Y. et al., in CN109608444.

In certain embodiments, the target protein ligand is a small organic molecule, such as having a molecular weight of less than 1500 Da, 1200 Da, 1000 Da, 800 Da, 600 Da, 400 Da, 300 Da, 200 Da, 150 Da, or 100 Da.

Where the target protein ligand is depicted as a discrete compound (e.g.,

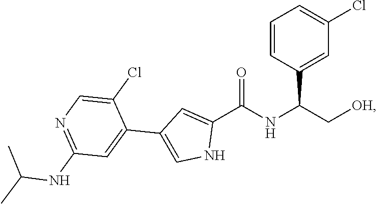 [SMILES: CC(C)Nc1cc(-c2c[nH]c(C(=O)N[C@H](CO)c3cccc(Cl)c3)c2)c(Cl)cn1]

it is understood that the target protein ligand is to be bonded to the linker via a modifiable carbon, oxygen, nitrogen, and/or sulfur atom present in the target protein ligand. Taking as an example the target protein ligand

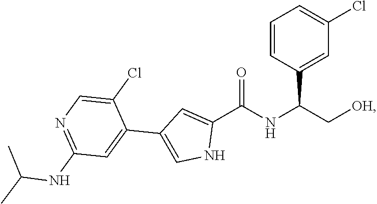 [SMILES: CC(C)Nc1cc(-c2c[nH]c(C(=O)N[C@H](CO)c3cccc(Cl)c3)c2)c(Cl)cn1]

the target protein ligand can be covalently bonded to the linker via the oxygen atom of the —OH group. The resulting linker-(target protein ligand) would have the following structure:

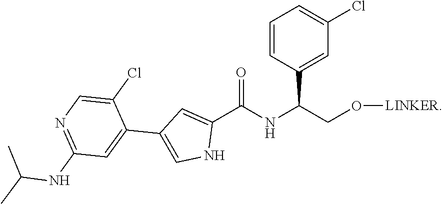

In certain embodiments, the target protein ligand is the target protein ligand component in one of the compounds set forth in Tables 1, 3, 4, 6, 7, 9, 11, 12, 14-16, or 18-20. In certain embodiments, the target protein ligand is the target protein ligand component in one of the compounds set forth in Tables 1, 3, 4, 6, 7, 9, 11, 12, 14-16, 18-20, or 22. In certain embodiments, the target protein ligand is the target protein ligand component in one of the compounds set forth in Tables 1, 3, 4, 6, 7, 9, 11, 12, 14A, 16A, or 22.

Linker

In one aspect, the compounds of the invention comprise a linker (“LINKER”). Any linker known in the art is useful within the invention. Non-limiting examples of linkers include amino acids, peptides, peptidomimetics, polyethylene glycols, polypropylene glycols, hydrocarbon-based chains (which may include alkyl chains, alkenyl chains, alkynyl chains, cycloalkyl chains, aryl chains, heteroaryl chains, heterocyclyl chains, and so on, and any combinations thereof).

Without wishing to be limited by any theory, the linker induces physical proximity between the phosphatase and the target protein. Binding of the (protein phosphatase ligand)-LINKER-(target protein ligand) to the phosphatase and the target protein leads to an increase in local concentration of the phosphatase with respect to the target protein (and, conversely, an increase in local concentration of the target protein with respect to the phosphatase), which allows for dephosphoarylation of the target protein by the phosphatase.

In certain embodiments, the linker is selected so that the compound of formula (I) [which is (protein phosphatase ligand)-LINKER-(target protein ligand), or a salt, solvate, prodrug, isotopically labelled derivative, stereoisomer, tautomer, or geometric isomer thereof, and any mixtures thereof] can simultaneously bind to the target protein (through the target protein ligand) and to the protein phosphatase (through the protein phosphatase ligand). In certain embodiments, in the compound of formula (I), the linker does not alter the binding affinity of the protein phosphatase ligand for the phosphatase and/or the binding affinity of the target protein ligand for the target protein. In certain embodiments, in the compound of formula (I), the linker does not significantly alter the binding affinity of the protein phosphatase ligand for the phosphatase and/or the binding affinity of the target protein ligand for the target protein. In certain embodiments, in the compound of formula (I), the linker enhances the binding affinity of the protein phosphatase ligand for the phosphatase and/or the binding affinity of the target protein ligand for the target protein. In some embodiments, the linker is symmetrical. In some embodiments, the linker is asymmetric.

In certain embodiments, the linker of the present invention is a bond.

In certain embodiments, the linker of the present invention has the formula:

—(CH₂)ₘ₁—X₄—(CH₂—CH₂—X₅)ₘ₂—(CH₂)ₘ₃—C(X₆)— (VI),

wherein the target protein ligand is covalently bonded to —(CH₂)ₘ₁, and the protein phosphatase ligand is covalently bonded to C(X₆)—. Alternatively, —(CH₂)ₘ₁ is covalently bonded to the protein phosphatase ligand, and C(X₆)— is covalently bonded to the target protein ligand. Each m1, m2, and m3 is independently 0, 1, 2, 3, 4, 5, 6, 7, 8, 9, or 10; each X₄, X₅, and X₆ is independently absent (a bond), O, S, or N—R²⁰, wherein each R²⁰ is independently selected from the group consisting of hydrogen, optionally substituted C₁-C₆ alkyl, optionally substituted aryl, optionally substituted heteroaryl, optionally substituted C₃-C₈ cycloalkyl, and optionally substituted C₃-C₈ cycloheteroalkyl.

In other embodiments, the linker of the present invention corresponds to formula:

—(CH₂)ₘ—O—(CH₂—H₂)(CH₂—O)—(CH)₃—C(O)— (VII),

wherein the target protein ligand is covalently bonded to —(CH₂)ₘ₁, and the protein phosphatase ligand is covalently bonded to C(O)—. Alternatively, —(CH₂)ₘ₁ is covalently bonded to the protein phosphatase ligand, and C(O)— is covalently bonded to the target protein ligand. Each m1, m2, and m3 is defined elsewhere herein.

In yet other embodiments, the linker of the present invention corresponds to formula:

—(CHR₂₁)ₘ₁—O—(CHR₂₂—CHR₂₃—O)ₘ₂—(CHR₂₄)ₘ₃—C(O)— (VIII),

wherein the protein phosphatase ligand is covalently bonded to —(CHR₂₁)ₘ₁, and the target protein ligand is covalently bonded to C(O)—. Alternatively, —(CHR₂₁)ₘ₁ is covalently bonded to the target protein ligand, and C(O)— is covalently bonded to the protein phosphatase ligand. Each m1, m2, and m3 is defined elsewhere herein; each R₂₁, R₂₂, R₂₃, and R₂₄ is independently selected from the group consisting of hydrogen, optionally substituted C₁-C₆ alkyl, optionally substituted aryl, optionally substituted heteroaryl, optionally substituted C₃-C₈ cycloalkyl, and optionally substituted C₃-C₈ cycloheteroalkyl.

In yet other embodiments, the linker of the present invention comprises a polyethylene glycol chain ranging in size from about 1 to about 12 ethylene glycol units, from about 1 to about 10 ethylene glycol units, from about 2 to about 6 ethylene glycol units, from about 2 to about 5 ethylene glycol units, or from about 2 to about 4 ethylene glycol units.

In additional embodiments, the linker group is optionally substituted (poly)ethyleneglycol having between 1 and about 100 ethylene glycol units, between about 1 and about 50 ethylene glycol units, between 1 and about 25 ethylene glycol units, between about 1 and about 10 ethylene glycol units, between 1 and about 8 ethylene glycol units, between 1 and about 6 ethylene glycol units, between 2 and about 4 ethylene glycol units, or optionally substituted alkyl groups interdispersed with optionally substituted, O, N, S, P or Si atoms. In certain embodiments, the linker is substituted with an aryl, phenyl, benzyl, alkyl, alkylene, or heterocycle group.

In yet other embodiments, the linker of the present invention corresponds to:

-(D-CON-D)ₘ₁- (IX),

wherein each D is independently a bond (absent), or —(CH₂)ₘ₁—Y—C(O)—Y—(CH₂)ₘ₁—; wherein m1 is defined elsewhere herein; Y is O, S or N—R⁴; CON is a bond (absent), an optionally substituted C₃-C₈ cycloheteroalkyl, piperazinyl or a group selected from the group consisting of the following chemical structures:

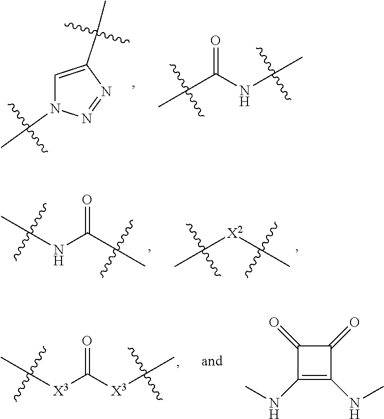

wherein X² is selected from the group consisting of O, S, NR⁴, S(O), S(O)₂, —S(O)₂O, —OS(O)₂, and OS(O)₂O;

X³ is selected from the group consisting of O, S, CHR⁴, and NR⁴; and

R⁴ is selected from the group consisting of H and a C₁-C₃ alkyl group optionally substituted with one or two hydroxyl groups.

In certain embodiments, the linker is selected from the group consisting of:
—NHCH₂CH₂(OCH₂CH₂)ₘOCH₂CH₂O—, wherein m is 0, 1, 2, 3, 4, 5, 6, 7, 8, 9, 10, 11, 12, 13, 14, 15, 16, 17, 18, 19, or 20;
—NHCH₂CH₂(OCH₂CH₂)ₘOCH₂CH₂O(CH₂)ₙ—, wherein m and n are independently selected from the group consisting of 0, 1, 2, 3, 4, 5, 6, 7, 8, 9, 10, 11, 12, 13, 14, 15, 16, 17, 18, 19, and 20; and
—(CH₂)ₙ₁(OCH₂CH₂)ₘ(CH₂)ₙ₂—, wherein m, n1, and n2 are independently selected from the group consisting of 0, 1, 2, 3, 4, 5, 6, 7, 8, 9, 10, 11, 12, 13, 14, 15, 16, 17, 18, 19, and 20.

In certain embodiments, the linker is a bivalent, saturated or unsaturated, straight or branched C₁₋₄₅ hydrocarbon chain, wherein 0-10 methylene units of the hydrocarbon are independently replaced with —O—, —S—, —N(R*)—, —OC(O)—, —C(O)O—, —S(O)—, —S(O)₂—, —N(R*)S(O)₂—, —S(O)₂N(R*)—, —N(R*)C(O)—, —C(O)N(R*)—, —OC(O)N(R*)—, —N(R*)C(O)O—, optionally substituted carbocyclyl, or optionally substituted heterocyclyl, wherein R* represents independently for each occurrence hydrogen, C₁₋₆ alkyl, or C₃₋₆ cycloalkyl.

In certain embodiments, the linker has the following formula:

—N(R)-(optionally substituted 3-20 membered heteroalkylene)ₚ-CH₂—C(O)—

wherein:
R is hydrogen or optionally substituted C₁-C₆ alkyl; and
p is 0 or 1.

In certain embodiments, the linker has the following formula:

—N(R)-(3-20 membered heteroalkylene)ₚ-CH₂—C(O)—

wherein:
the 3-20 membered heteroalkylene is optionally substituted with 1, 2, 3, or 4 substituents independently selected from halogen, C₁-C₆ haloalkyl, C₃-C₆ cycloalkyl, hydroxyl, and cyano;
R is hydrogen or optionally substituted C₁-C₆ alkyl; and
p is 0 or 1.

In certain embodiments, the linker has the following formula:

—N(R)-(3-20 membered heteroalkylene)ₚ-CH₂—C(O)—

wherein:
the 3-20 membered heteroalkylene is optionally substituted with 1, 2, or 3 substituents independently selected from halogen and C₁-C₆ haloalkyl;
R is hydrogen or C₁-C₆ alkyl; and
p is 0 or 1.

In certain embodiments, the linker has the following formula:

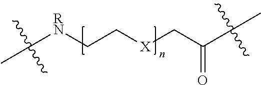

wherein:
X represents independently for each occurrence a bond, —O—, or —N(R¹)—;
R¹ represents independently for each occurrence hydrogen or optionally substituted C₁-C₆ alkyl;
R is hydrogen or optionally substituted C₁-C₆ alkyl; and
n is 0, 1, 2, 3, or 4.

In certain embodiments, the linker has the following formula:

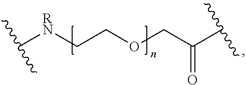

wherein R is hydrogen or optionally substituted C₁-C₆ alkyl, and n is 0, 1, 2, 3, or 4.

In some embodiments, R is hydrogen or C₁-C₆ alkyl.

In certain embodiments, the linker has the following formula:

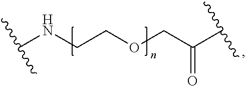

wherein n is 0, 1, 2, 3, or 4.

In some embodiments, n is 0. In some embodiments, n is 1. In some embodiments, n is 2. In some embodiments, n is 3. In some embodiments, n is 4. In some embodiments, n is 0 or 1. In some embodiments, n is 1 or 2. In some embodiments, n is 2 or 3. In some embodiments, n is 3 or 4. In some embodiments, n is 0, 1, or 2. In some embodiments, n is 1, 2, or 3. In some embodiments, n is 2, 3, or 4. In some embodiments, n is 0, 1, 2, or 3. In some embodiments, n is 1, 2, 3, or 4.

In certain embodiments, the linker has the following formula:

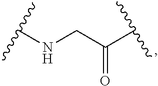 [SMILES: *NCC(*)=O]

In certain embodiments, the linker has the following formula:

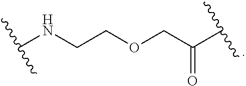 [SMILES: *NCCOCC(*)=O]

In certain embodiments, the linker has the following formula:

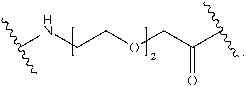

In certain embodiments, the linker has the following formula:

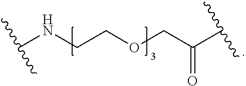

In certain embodiments, the linker has the following formula:

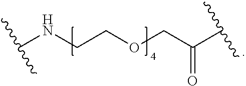

In certain embodiments, the linker has the following formula:

—C(O)-(optionally substituted C₀-C₅ alkylene)-C(O)—N(R)-(optionally substituted 3-20 membered heteroalkylene)ₚ-CH₂—C(O)—

wherein:
R is hydrogen or optionally substituted C₁-C₆ alkyl; and
p is 0 or 1.
   In certain embodiments, the linker has the following formula:

—C(O)—(C₀-C₅ alkylene)-C(O)—N(R)-(3-20 membered heteroalkylene)ₚ-CH₂—C(O)—

wherein:
the C₀-C₅ alkylene and 3-20 membered heteroalkylene are each optionally substituted with 1, 2, or 3 substituents independently selected from halogen, C₁-C₆ haloalkyl, C₃-C₆ cycloalkyl, hydroxyl, C₁-C₆ alkoxy, and cyano;
R is hydrogen or optionally substituted C₁-C₆ alkyl; and
p is 0 or 1.

In certain embodiments, the linker has the following formula:

—C(O)—(C₀-C₅ alkylene)-C(O)—N(R)-(3-20 membered heteroalkylene)ₚ-CH₂—C(O)—

wherein:

R is hydrogen or C₁-C₆ alkyl; and

p is 0 or 1.

In certain embodiments, the linker has the following formula:

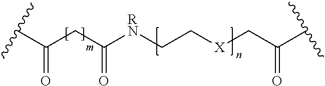

wherein:
X represents independently for each occurrence a bond, —O—, or —N(R¹)—;
R¹ represents independently for each occurrence hydrogen or optionally substituted C₁-C₆ alkyl;
R is hydrogen or optionally substituted C₁-C₆ alkyl; and
m and n are independently 0, 1, 2, 3, or 4.

In certain embodiments, the linker has the following formula:

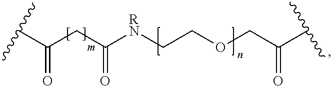

wherein R is hydrogen or optionally substituted C₁-C₆ alkyl, and m and n are independently 0, 1, 2, 3, or 4.

In some embodiments, R is hydrogen or C₁-C₆ alkyl.

In some embodiments, m is 0. In some embodiments, m is 1. In some embodiments, m is 2. In some embodiments, m is 3. In some embodiments, m is 4. In some embodiments, m is 0 or 1. In some embodiments, m is 1 or 2. In some embodiments, m is 2 or 3. In some embodiments, m is 3 or 4. In some embodiments, m is 0, 1, or 2. In some embodiments, m is 1, 2, or 3. In some embodiments, m is 2, 3, or 4. In some embodiments, m is 0, 1, 2, or 3. In some embodiments, m is 1, 2, 3, or 4.

In certain embodiments, the linker has the following formula:

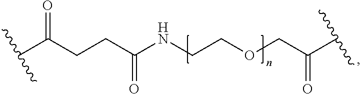

wherein n is 0, 1, 2, 3, or 4.

In some embodiments, n is 0. In some embodiments, n is 1. In some embodiments, n is 2. In some embodiments, n is 3. In some embodiments, n is 4. In some embodiments, n is 0 or 1. In some embodiments, n is 1 or 2. In some embodiments, n is 2 or 3. In some embodiments, n is 3 or 4. In some embodiments, n is 0, 1, or 2. In some embodiments, n is 1, 2, or 3. In some embodiments, n is 2, 3, or 4. In some embodiments, n is 0, 1, 2, or 3. In some embodiments, n is 1, 2, 3, or 4.

In certain embodiments, the linker has the following formula:

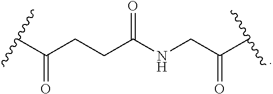 [SMILES: *C(=O)CCC(=O)NCC(*)=O]

In certain embodiments, the linker has the following formula:

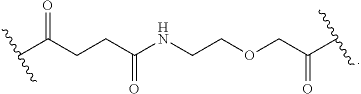 [SMILES: *C(=O)CCC(=O)NCCOCC(*)=O]

In certain embodiments, the linker has the following formula:

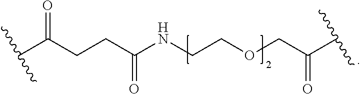

In certain embodiments, the linker has the following formula:

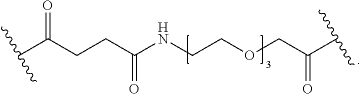

In certain embodiments, the linker has the following formula:

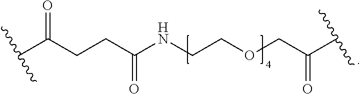

In certain embodiments, the linker has the following formula:

—CH₂-(optionally substituted C₀-C₅ alkylene)-C(O)—N(R)-(optionally substituted 3-20 membered heteroalkylene)ₚ-CH₂—C(O)—

wherein:
R is hydrogen or optionally substituted C₁-C₆ alkyl; and
p is 0 or 1.

In certain embodiments, the linker has the following formula:

—CH₂—(C₀-C₅ alkylene)-C(O)—N(R)-(3-20 membered heteroalkylene)ₚ-CH₂—C(O)—

wherein:
the C₀-C₅ alkylene and 3-20 membered heteroalkylene are each optionally substituted with 1, 2, or 3 substituents independently selected from halogen, C₁-C₆ haloalkyl, C₃-C₆ cycloalkyl, hydroxyl, C₁-C₆ alkoxy, and cyano;
R is hydrogen or optionally substituted C₁-C₆ alkyl; and
p is 0 or 1.

In certain embodiments, the linker has the following formula:

—CH₂—(C₀-C₅ alkylene)-C(O)—N(R)-(3-20 membered heteroalkylene)ₚ-CH₂—C(O)—

wherein:
R is hydrogen or C₁-C₆ alkyl; and
p is 0 or 1.

In certain embodiments, the linker has the following formula:

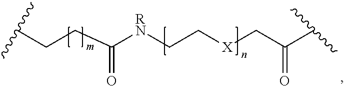

wherein:
X represents independently for each occurrence a bond, —O—, or —N(R′)—;
R¹ represents independently for each occurrence hydrogen or optionally substituted C₁-C₆ alkyl;
R is hydrogen or optionally substituted C₁-C₆ alkyl; and
m and n are independently 0, 1, 2, 3, or 4.

In certain embodiments, the linker has the following formula:

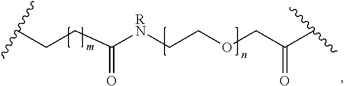

wherein R is hydrogen or optionally substituted C₁-C₆ alkyl, and m and n are independently 0, 1, 2, 3, or 4.

In some embodiments, R is hydrogen or C₁-C₆ alkyl.

In some embodiments, m is 0. In some embodiments, m is 1. In some embodiments, m is 2. In some embodiments, m is 3. In some embodiments, m is 4. In some embodiments, m is 0 or 1. In some embodiments, m is 1 or 2. In some embodiments, m is 2 or 3. In some embodiments, m is 3 or 4. In some embodiments, m is 0, 1, or 2. In some embodiments, m is 1, 2, or 3. In some embodiments, m is 2, 3, or 4. In some embodiments, m is 0, 1, 2, or 3. In some embodiments, m is 1, 2, 3, or 4.

In certain embodiments, the linker has the following formula:

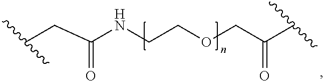

wherein n is 0, 1, 2, 3, or 4.

In some embodiments, n is 0. In some embodiments, n is 1. In some embodiments, n is 2. In some embodiments, n is 3. In some embodiments, n is 4. In some embodiments, n is 0 or 1. In some embodiments, n is 1 or 2. In some embodiments, n is 2 or 3. In some embodiments, n is 3 or 4. In some embodiments, n is 0, 1, or 2. In some embodiments, n is 1, 2, or 3. In some embodiments, n is 2, 3, or 4. In some embodiments, n is 0, 1, 2, or 3. In some embodiments, n is 1, 2, 3, or 4.

In certain embodiments, the linker has the following formula:

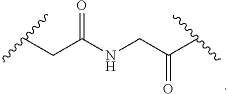 [SMILES: *CC(=O)NCC(*)=O]

In certain embodiments, the linker has the following formula:

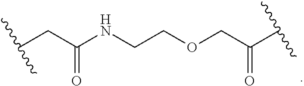 [SMILES: *CC(=O)NCCOCC(*)=O]

In certain embodiments, the linker has the following formula:

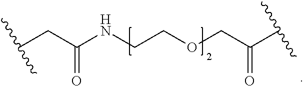

In certain embodiments, the linker has the following formula:

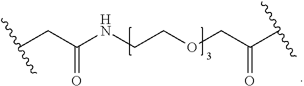

In certain embodiments, the linker has the following formula:

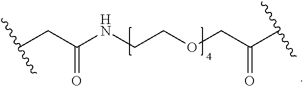

In certain embodiments, the LINKER is one of the following:

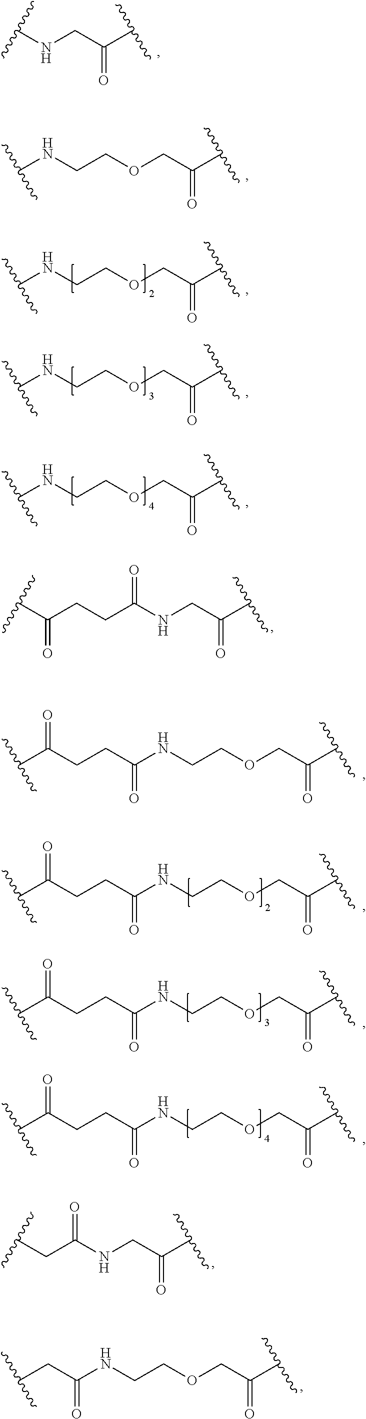

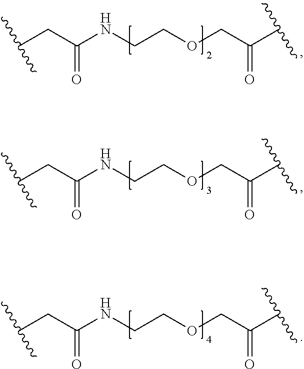

In certain embodiments, the LINKER is:

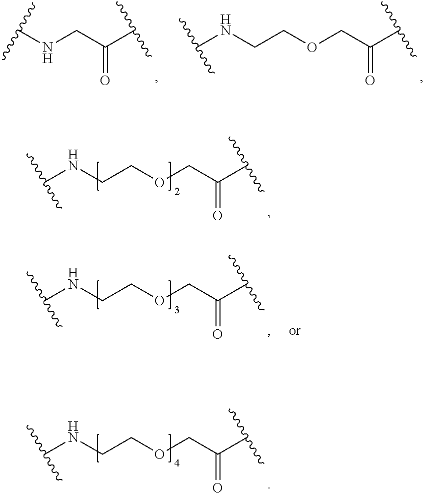

In some embodiments, the linker corresponds to formula:

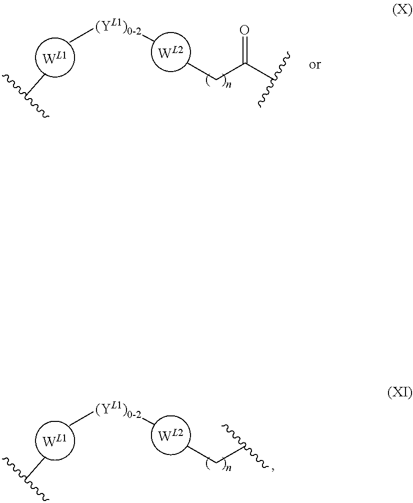

wherein:
the symbol “ [symbol] ” indicates a point of attachment to the protein phosphatase ligand or the target protein ligand;
W^{L}1 and W^{L2} are each independently a 4-8 membered ring with 0-4 heteroatoms, optionally substituted with R^{Q}; wherein each R^{Q} is independently a H, halo, OH, CN, CF₃, optionally substituted C₁-C₆ alkyl, optionally substituted C₁-C₆ alkoxy, or 2 R^{Q} groups are taken together with the atom they are attached to, form a 4-8 membered ring system containing 0-4 heteroatoms;
Y^{L1} is each independently a bond, optionally substituted C₁-C₆ alkyl, or optionally substituted 2-8 membered heteroalkyl (e.g., C₁-C₆ alkoxy; and
n is 0-10.

In some embodiments, the linker corresponds to formula:

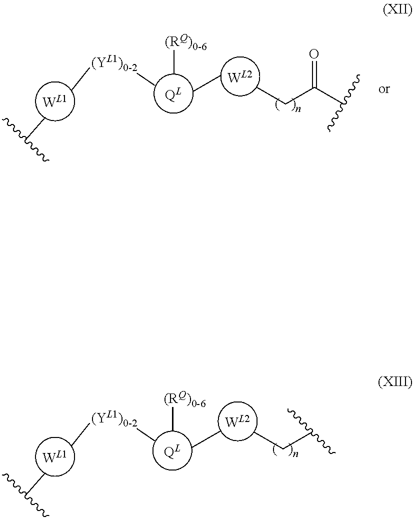

### Wherein:

the symbol “ [symbol] ” indicates a point of attachment to the protein phosphatase ligand or the target protein ligand;
W^{L1} and W^{L2} are each independently aryl, heteroaryl, cyclic, heterocyclic, C₁₋₆ alkyl, bicyclic, biaryl, biheteroaryl, or biheterocyclic, each optionally substituted with R^{Q}; wherein each R^{Q} is independently a H, halo, OH, CN, CF₃, hydroxyl, nitro, C≡CH, C₂₋₆ alkenyl, C₂₋₆ alkynyl, optionally substituted C₁-C₆ alkyl, optionally substituted C₁-C₆ alkoxy, optionally substituted O—C₁₋₃alkyl (e.g., C₁₋₃ haloalkoxyl), OH, NH₂, NR^{Y1}R^{Y2} CN, or 2 R^{Q} groups are taken together with the atom they are attached to, form a 4-8 membered ring system containing 0-4 heteroatoms;
Y^{L1} is each independently a bond, NR^{YL1}, O, S, NR^{YL2}, CR^{YL1}R^{YL2}, C═O, C═S, SO, SO₂, optionally substituted C₁-C₆ alkyl, or optionally substituted C₁-C₆ alkoxy;
Q^{L} is a 3-6 membered alicyclic or aromatic ring with 0-4 heteroatoms, optionally bridged, optionally substituted with 0-6 R^{Q}; wherein each R^{Q} is independently H, optionally substituted C₁₋₆ alkyl, optionally substituted C₁₋₆ alkoxyl, or 2 R^{Q} groups are taken together with the atom they are attached to, form a 3-8 membered ring system containing 0-2 heteroatoms);
R^{YL1}, R_{YL2} are each independently H, OH, optionally substituted C₁₋₆ alkyl, or R¹, R² together with the atom they are attached to, form a 3-8 membered ring system containing 0-2 heteroatoms); and
n is 0-10.

In some embodiments, the linker is a group comprising one or more covalently connected structural units independently selected from the group consisting of:

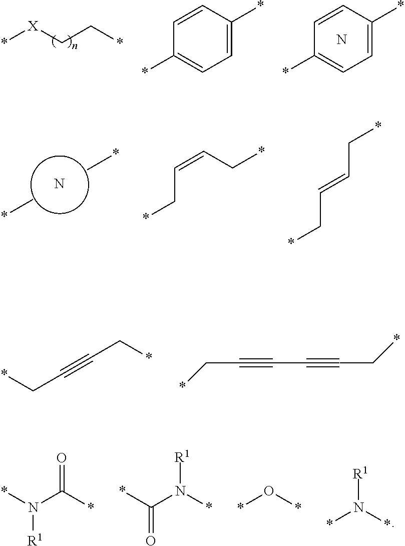

wherein:
indicates a point of attachment to another structural unit, the protein phosphatase ligand, or the target protein ligand;
X is selected from the group consisting of O, N, S, S(O) and SO₂;
n is an integer from 1 to 5;
R¹ in the linker is hydrogen or alkyl;

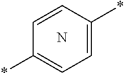

is a mono- or bicyclic aryl or heteroaryl optionally substituted with 1-3 substituents selected from alkyl, halogen, haloalkyl, hydroxy, alkoxy or cyano;

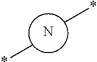

is a mono- or bicyclic cycloalkyl or a heterocycloalkyl optionally substituted with 1-3 substituents selected from alkyl, halogen, haloalkyl, hydroxy, alkoxy or cyano; and

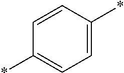 [SMILES: *c1ccc(*)cc1]

is optionally substituted with 1, 2 or 3 substituents selected from the group consisting of alkyl, halogen, haloalkyl, hydroxy, alkoxy, amino, and cyano.

In some embodiments, the linker comprises up to 10 covalently connected structural units, as described above.

In some embodiments, the linker is selected from the group consisting of:

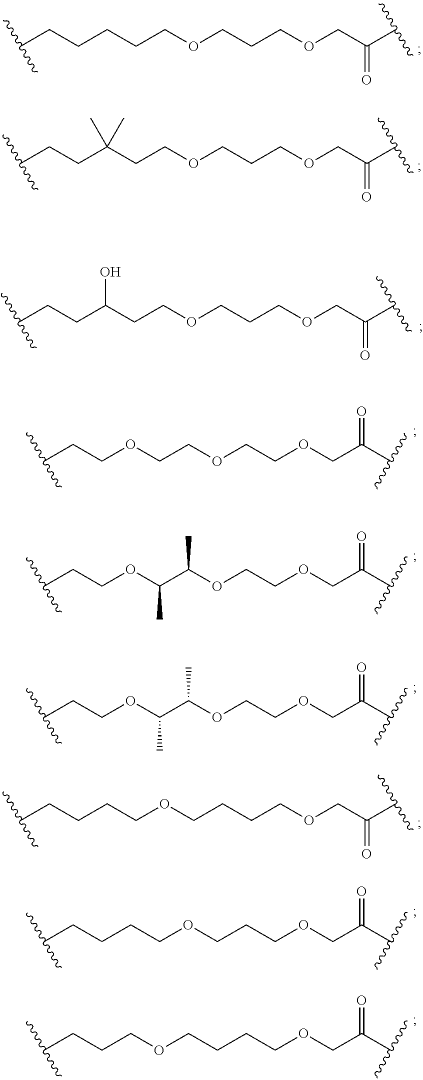

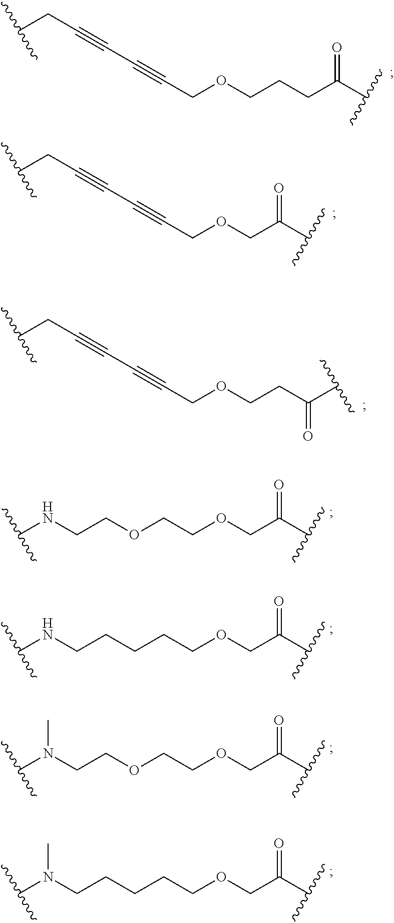

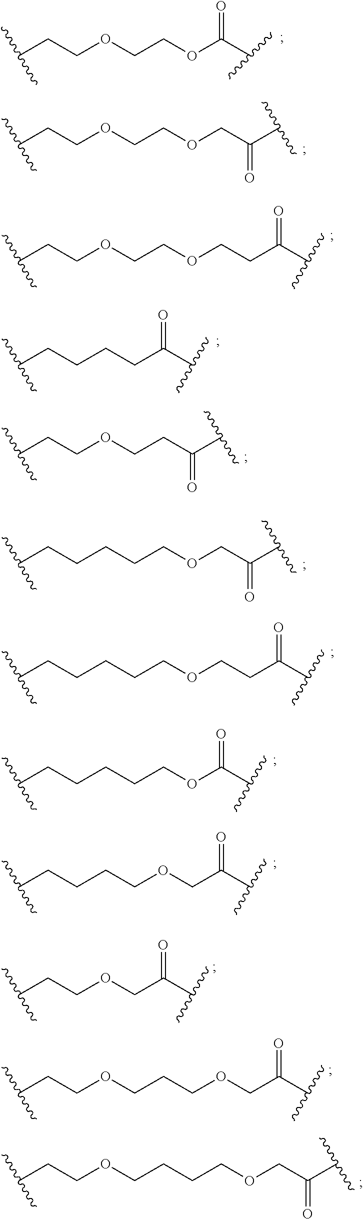

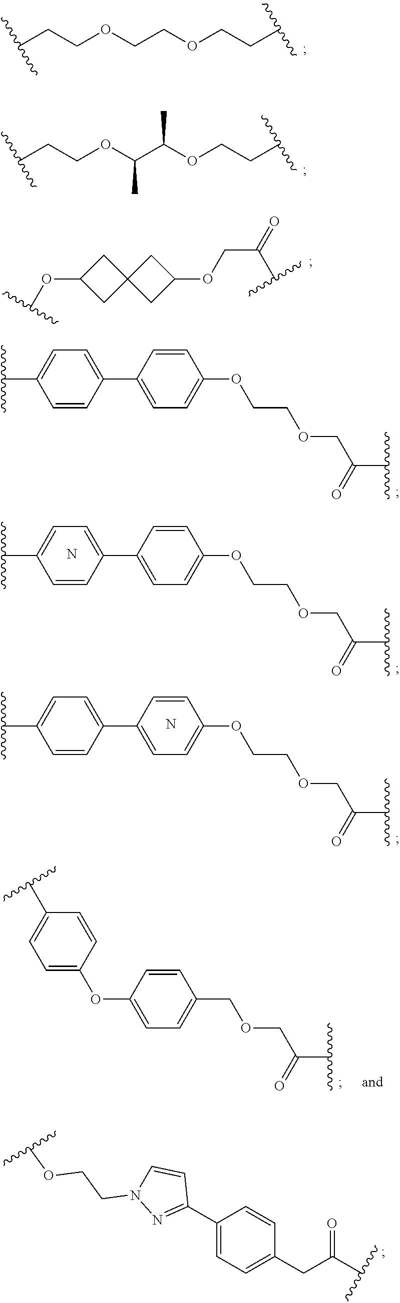

wherein the symbol “ [symbol] ” indicates a point of attachment to the protein phosphatase ligand or the target protein ligand.

In certain embodiments, the linker is -(A^{L})_{q}-, wherein:
q is an integer greater than or equal to 1 (e.g., 1, 2, 3, 4, 5, 6, 7, 8, 9 or 10);
each A^{L} is independently selected from the group consisting of a bond, CR^{L1}R^{L2}, O, S, SO, SO², NR^{L3}, SO₂NR^{L3}, SONR^{L3}, CONR^{L3}, NR^{L3}CONR^{L4}, NR^{L3}SO₂NR^{L4}, CO, CR^{L1}═CR^{L2}, C≡C, SiR^{L1}R^{L2}, P(O)R^{L1}, P(O)OR^{L1}, NR^{L3}C(═NCN)NR^{L4}, NR^{L3}C(═NCN), NR^{L3}C(═CNO₂)NR^{L4}, C₃₋₁₁cycloalkyl optionally substituted with 0-6 R^{L1} and/or R^{L2} groups, C₅₋₁₃ spirocycloalkyl optionally substituted with 0-9 R^{L} and/or R^{L2} groups, C₃₋₁₁heterocyclyl optionally substituted with 0-6 R^{L1} and/or R^{L2} groups, C₅₋₁₃ spiroheterocycloalkyl optionally substituted with 0-8 R^{L1} and/or R^{L2} groups, aryl optionally substituted with 0-6 R^{L1} and/or R^{L2} groups, heteroaryl optionally substituted with 0-6 R^{L1} and/or R^{L2} groups, where R^{L1} or R^{L2}, each independently are optionally linked to other groups to form cycloalkyl and/or heterocyclyl moiety, optionally substituted with 0-4 R^{L5} groups; and R^{L1}, R^{L2}, R^{L3}, R^{L4} and R^{L5} are, each independently, H, halo, C₁₋₈alkyl, OC₁₋₈alkyl, SC₁₋₈alkyl, NHC₁₋₈alkyl, N(C₁₋₈alkyl)₂, C₃₋₁₁cycloalkyl, aryl, heteroaryl, C₃₋₁₁heterocyclyl, OC₁₋₈cycloalkyl, SC₁₋₈cycloalkyl, NHC₁₋₈cycloalkyl, N(C₁₋₈cycloalkyl)₂, N(C₁₋₈cycloalkyl)(C₁₋₈alkyl), OH, NH₂, SH, SO₂C₁₋₈alkyl, P(O)(OC₁₋₈alkyl)(C₁₋₈alkyl), P(O)(OC₁₋₈alkyl)₂, CC—C₁₋₈alkyl, CCH, CH═CH(C₁₋₈alkyl), C(C₁₋₈alkyl)═CH(C₁₋₈alkyl), C(C₁₋₈alkyl)═C(C₁₋₈alkyl)₂, Si(OH)₃, Si(C₁₋₈alkyl)₃, Si(OH)(C₁₋₈alkyl)₂, COC₁₋₈alkyl, CO₂H, halogen, CN, CF₃, CHF₂, CH₂F, NO₂, SF₅, SO₂NHC₁₋₈alkyl, SO₂N(C₁₋₈alkyl)₂, SONHC₁₋₈alkyl, SON(C₁₋₈alkyl)₂, CONHC₁₋₈alkyl, CON(C₁₋₈alkyl)₂, N(C₁₋₈alkyl)CONH(C₁₋₈alkyl), N(C₁₋₈alkyl)CON(C₁₋₈alkyl)₂, NHCONH(C₁₋₈alkyl), NHCON(C₁₋₈alkyl)₂, NHCONH₂, N(C₁₋₈alkyl)SO₂NH(C₁₋₈alkyl), N(C₁₋₈alkyl) SO₂N(C₁₋₈alkyl)₂, NH SO₂NH(C₁₋₈alkyl), NH SO₂N(C₁_alkyl)₂, or NH SO₂NH₂.

In some embodiments, q is 1 to 2. In some embodiments, q is 1 to 5. In some embodiments, q is 1 to 10. In some embodiments, q is 1 to 20. In some embodiments, q is 1, 2, 3, 4, 5, 6, 7, 8, 9, 10, 11, 12, 13, 14, 15, 16, 17, 18, 19, or 20. In some embodiments, q is an integer from 1 to 100, 1 to 90, 1 to 80, 1 to 70, 1 to 60, 1 to 50, 1 to 40, or 1 to 30.

In some embodiments, the linker is selected from the group consisting of —NR(CH₂)ₙ-(lower alkyl)-, —NR(CH₂)ₙ-(lower alkoxyl)-, —NR(CH₂)ₙ-(lower alkoxyl)-OCH₂—, —NR(CH₂)ₙ-(lower alkoxyl)-(lower alkyl)-OCH₂—, —NR(CH₂)ₙ-(cycloalkyl)-(lower alkyl)-OCH₂—, —NR(CH₂)ₙ-(hetero cycloalkyl)-, —NR(CH₂CH₂O)ₙ-(lower alkyl)-O—CH₂—, —NR(CH₂CH₂O)ₙ-(hetero cycloalkyl)-O—CH₂—, —NR(CH₂CH₂O)ₙ-Aryl-O—CH₂—, —NR(CH₂CH₂O)ₙ-(hetero aryl)-O—CH₂—, —NR(CH₂CH₂O)ₙ-(cyclo alkyl)-O-(hetero aryl)-O—CH₂—, —NR(CH₂CH₂O)ₙ-(cyclo alkyl)-O-Aryl-O—CH₂—, —NR(CH₂CH₂O)ₙ-(lower alkyl)-NH-Aryl-O—CH₂—, —NR(CH₂CH₂O)ₙ-(lower alkyl)-O-Aryl-CH₂, —NR(CH₂CH₂O)ₙ-cycloalkyl-O-Aryl-, —NR(CH₂CH₂O)ₙ-cycloalkyl-O-(heteroaryl)1-, —NR(CH₂CH₂)ₙ-(cycloalkyl)-O-(heterocycle)-CH₂, —NR(CH₂CH₂)ₙ-(heterocycle)-(heterocycle)-CH₂, —N(R1R2)-(heterocycle)-CH₂; wherein n of the linker can be 0 to 10; R of the linker can be H, lower alkyl; and R1 and R2 of the linker can form a ring with the connecting N.

In some embodiments, the linker is selected from the group consisting of: —N(R)—(CH₂)ₘ—O(CH₂)ₙ—O(CH₂)ₒ—O(CH₂)ₚ—O(CH₂)_{q}—O(CH₂)ᵣ—OCH₂—, —O—(CH₂)ₘ—O(CH₂)ₙ—O(CH₂)ₒ—O(CH₂)ₚ—O(CH₂)O(CH₂)ᵣ—OCH₂—, —O—(CH₂)ₘ—O(CH₂)ₙ—O(CH₂)ₒ—O(CH₂)ₚ—O(CH₂)O(CH₂)ᵣ—O—; —N(R)—CH₂)ₘ(CH₂)ₙ—O(CH₂) (CH₂)ₚ—O(CH₂)O(CH₂)ᵣ—O—; —(CH₂)ₘ—O((CH₂)ₙ—O(CH₂)ₒ—O(CH₂)ₚ—O(CH₂)_{q}—O(CH₂)ᵣ—O—; —(CH₂)ₘ—O(CH₂)ₙ—O(CH₂)ₒ—O(CH₂)ₚ—O(CH₂)_{q}—O(CH₂)ᵣ—OCH₂—;

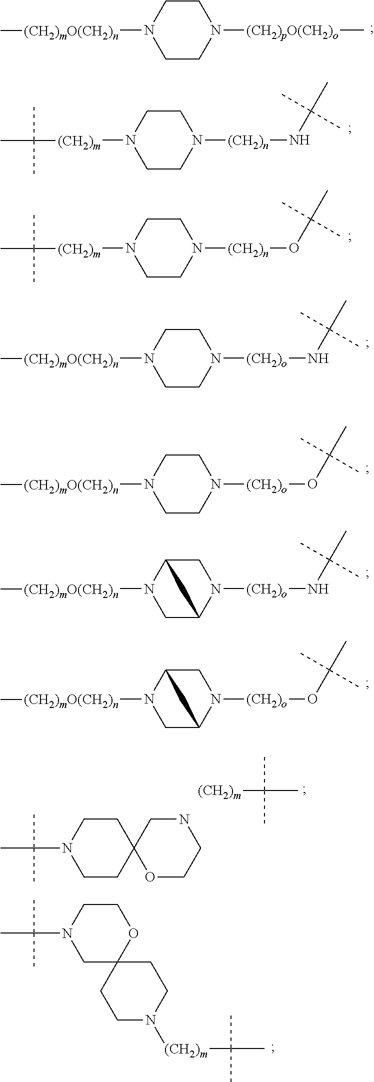

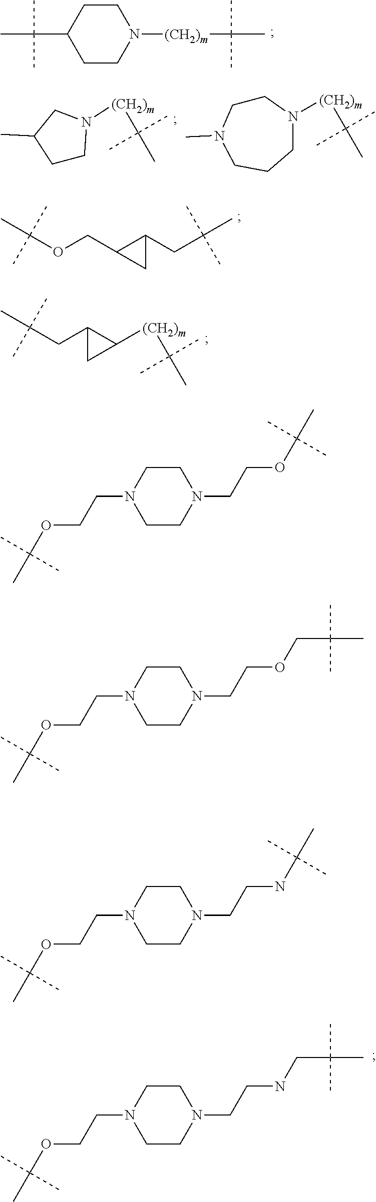

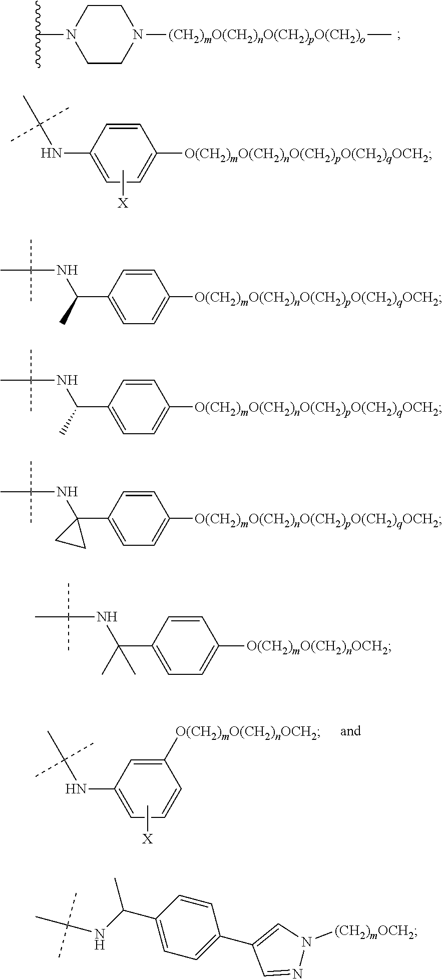

wherein
m, n, o, p, q, and r of the linker are independently 1, 2, 3, 4, 5, 6; 7, 8, 9, 10, 11, 12, 13, 14, 15, 16, 17, 18, 19, or 20;
R of the linker is H, methyl, or ethyl; and
X of the linker is H or F.

In some embodiments, the linker is selected from the group consisting of:

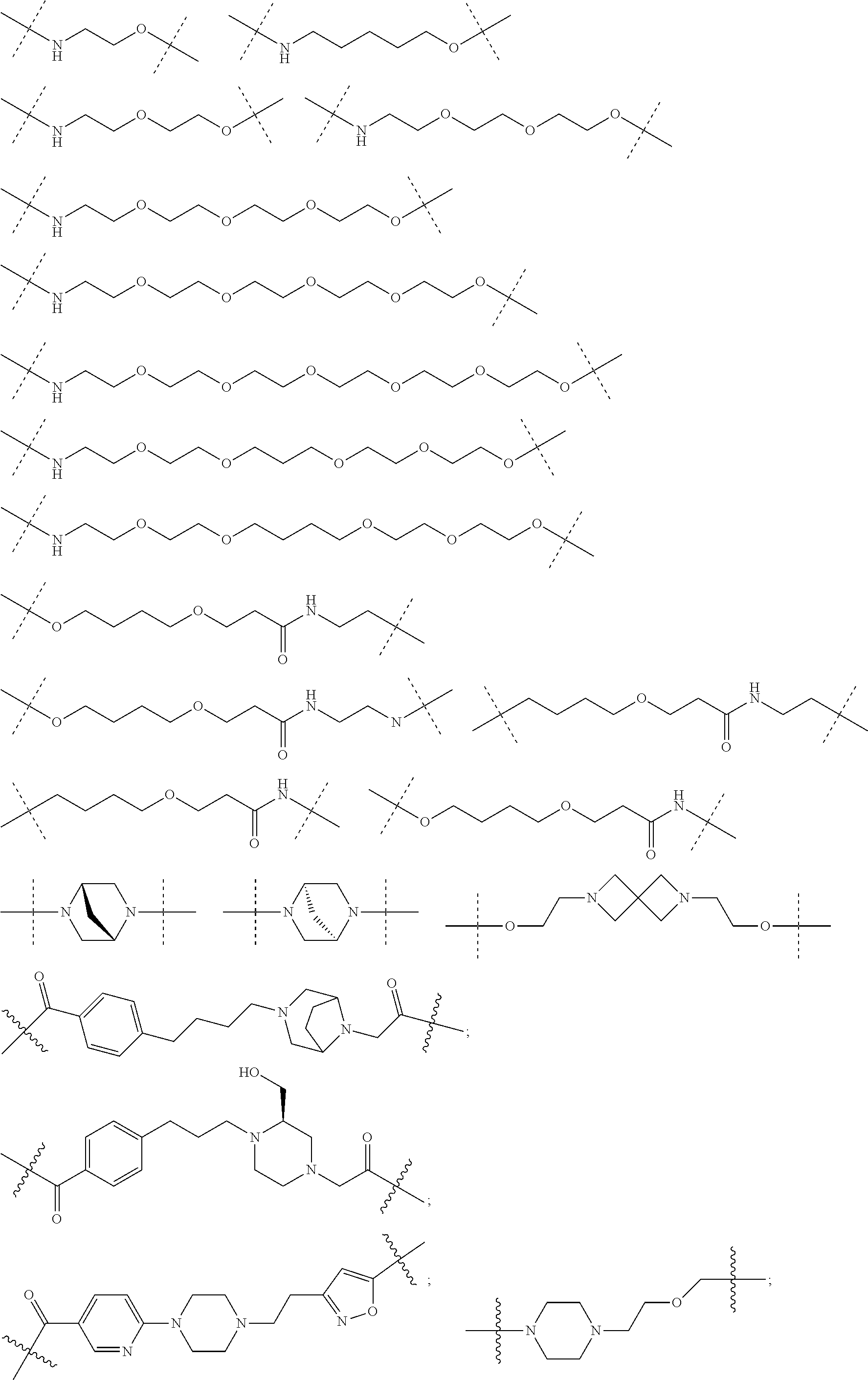

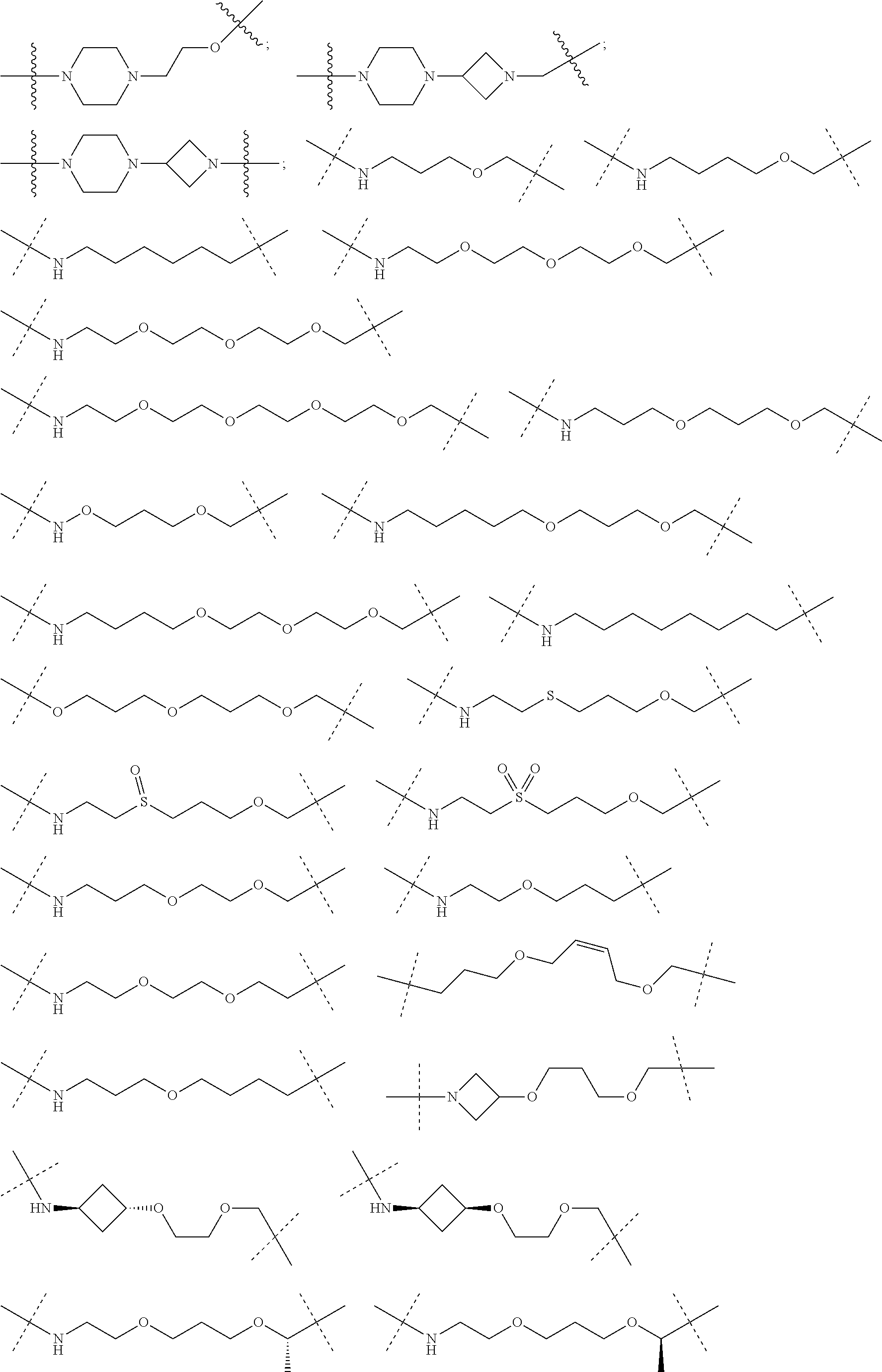

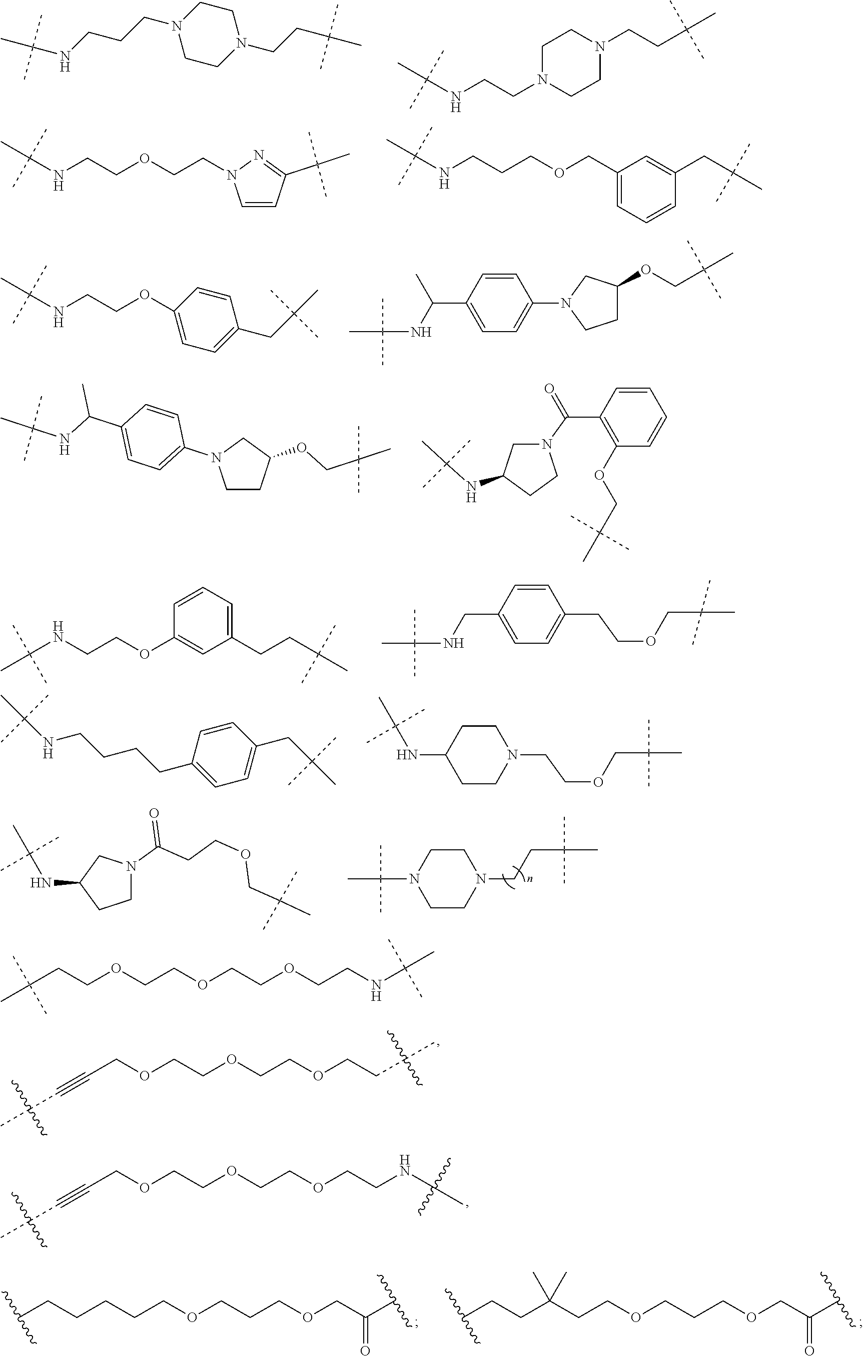

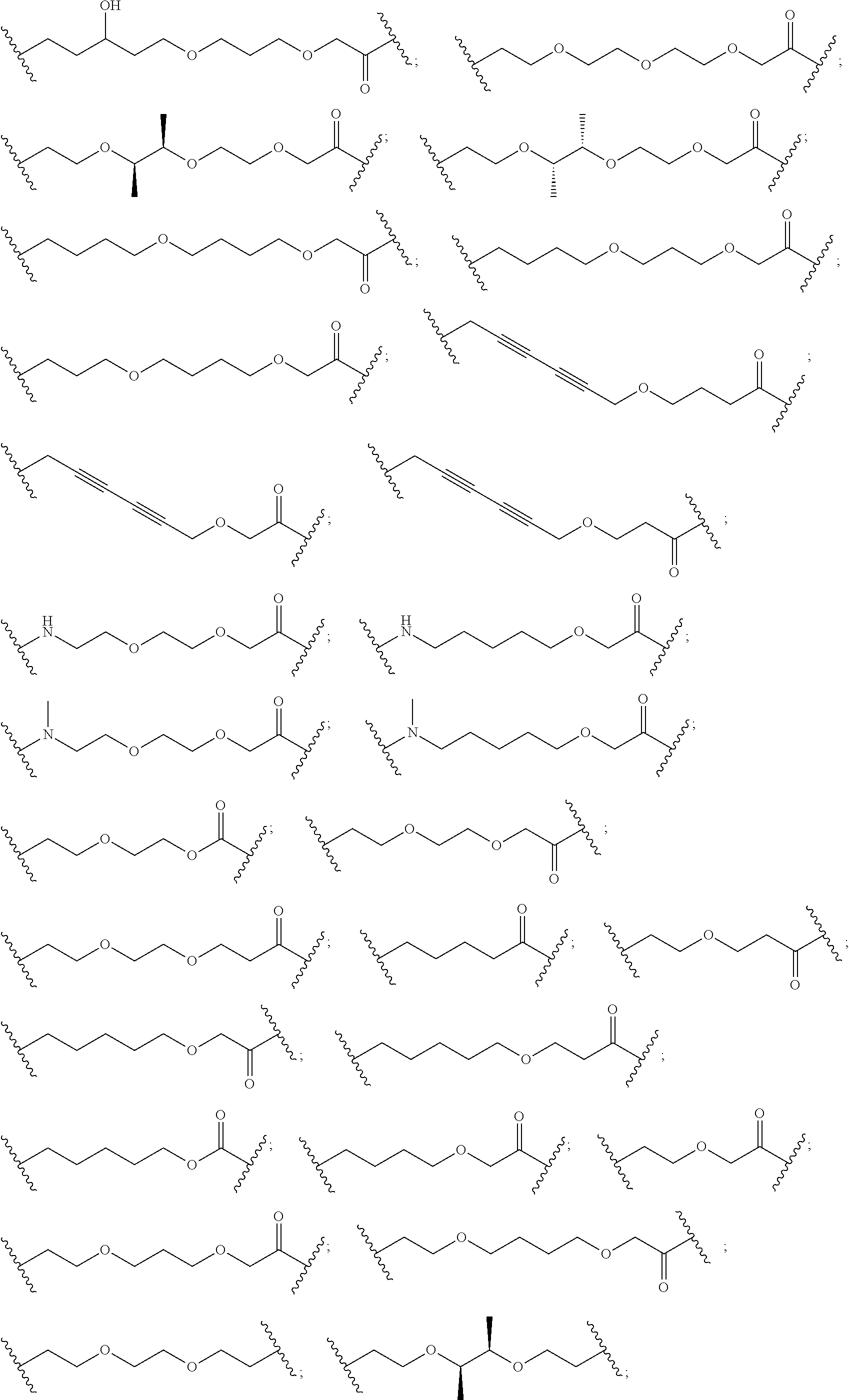

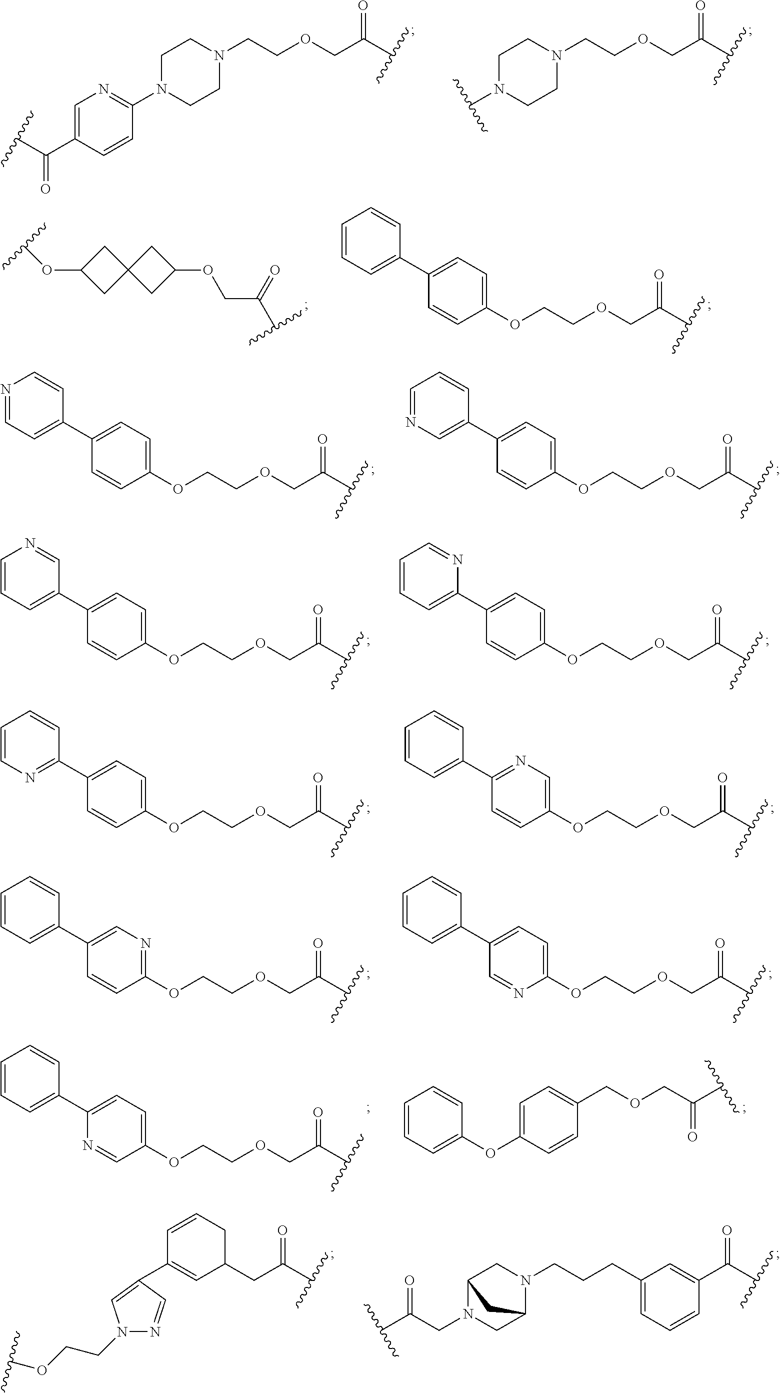

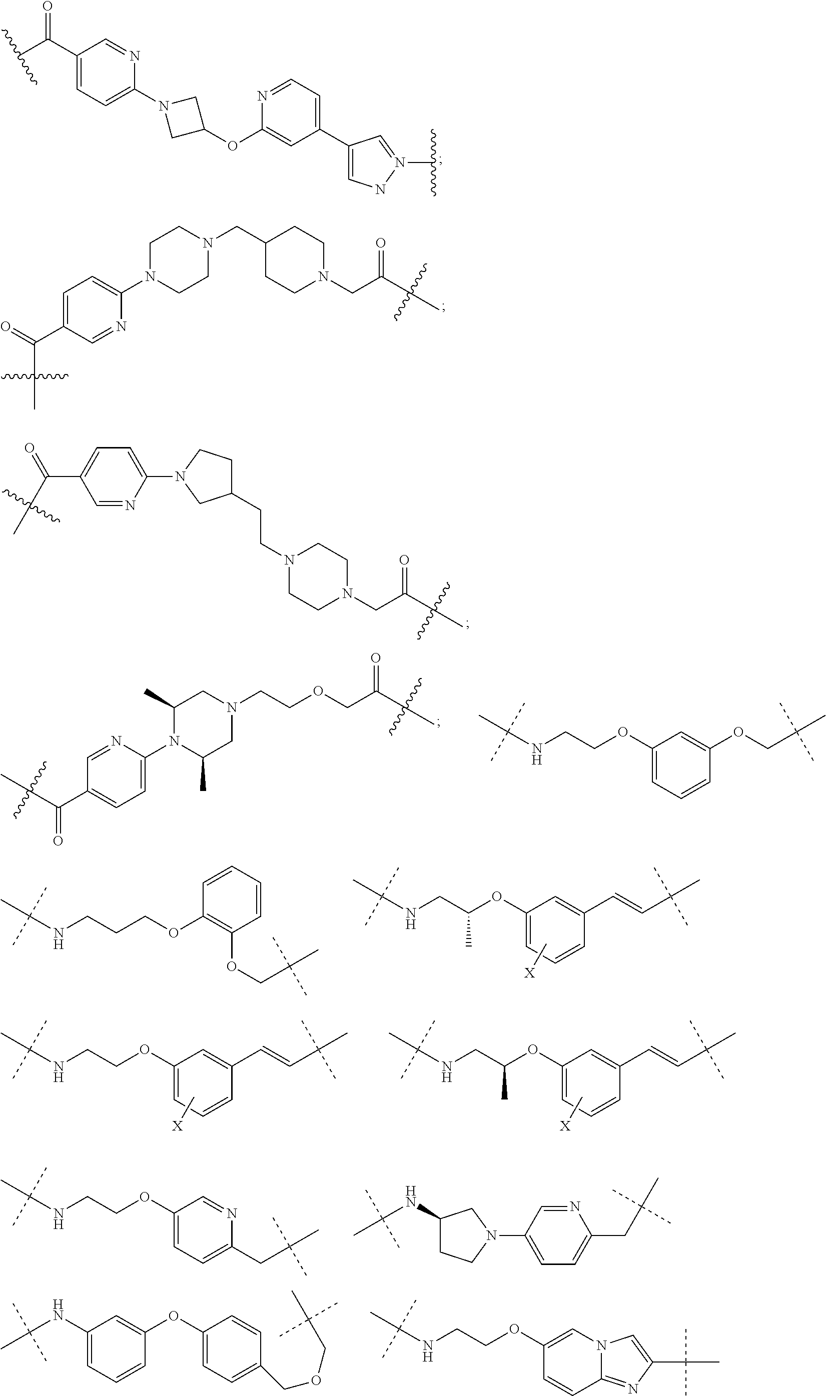

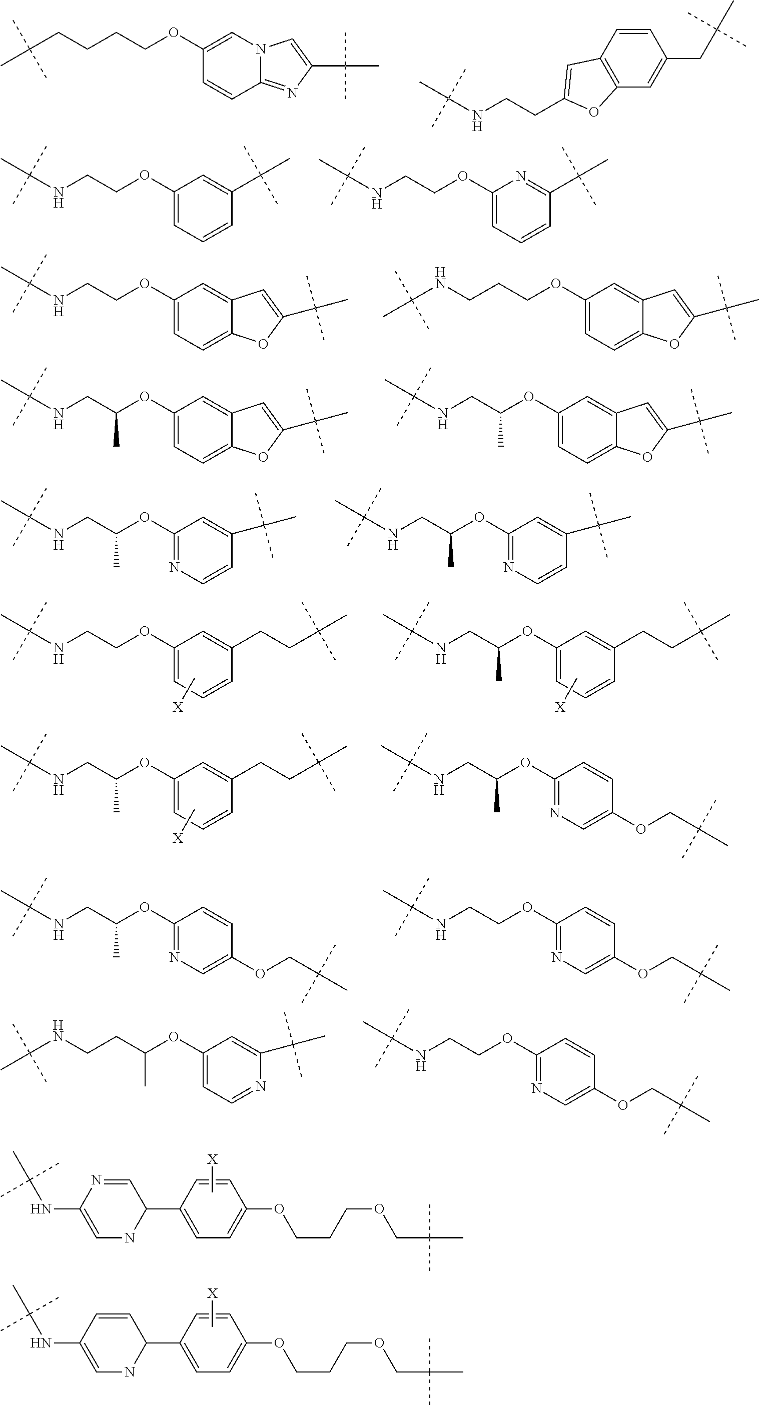

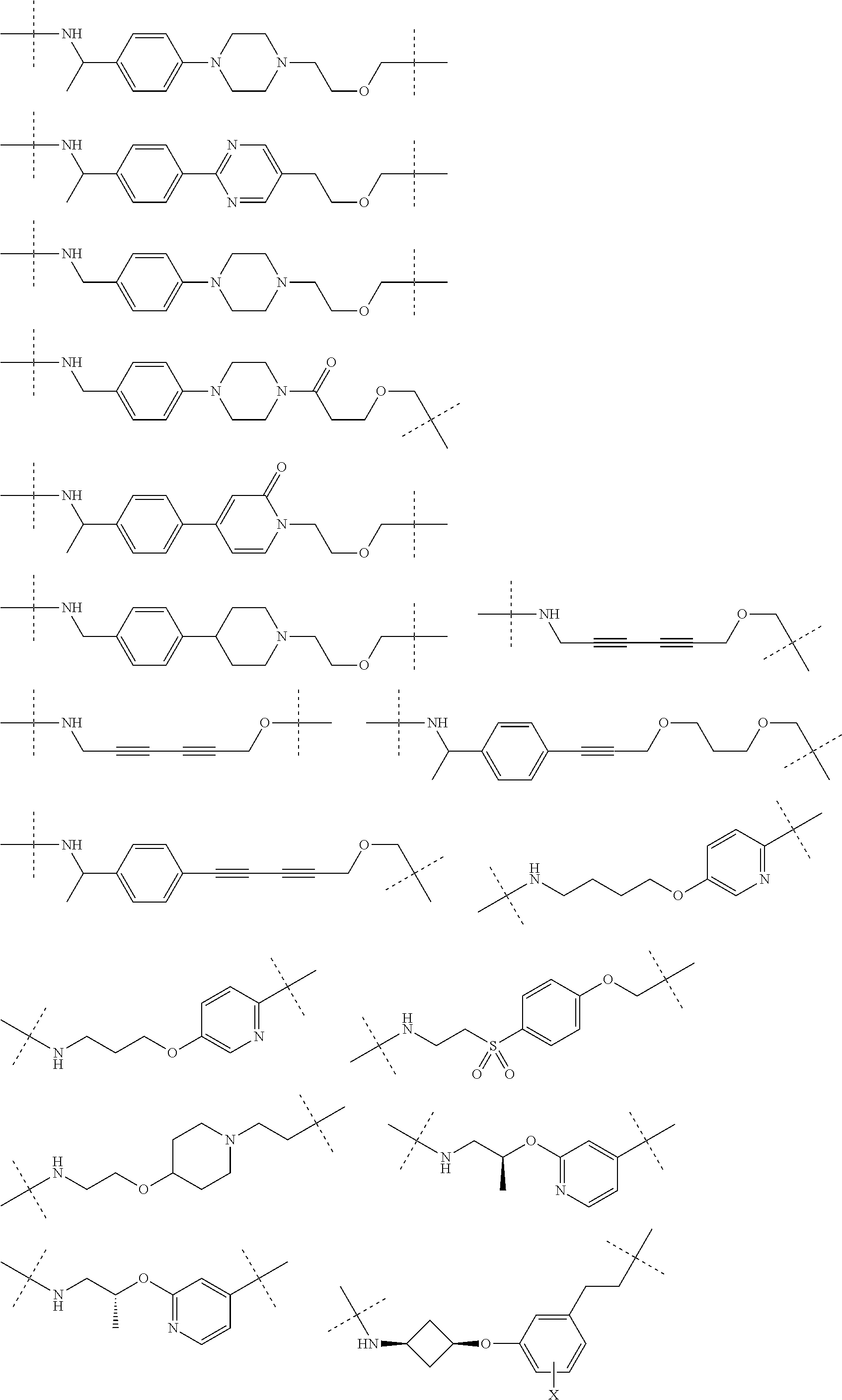

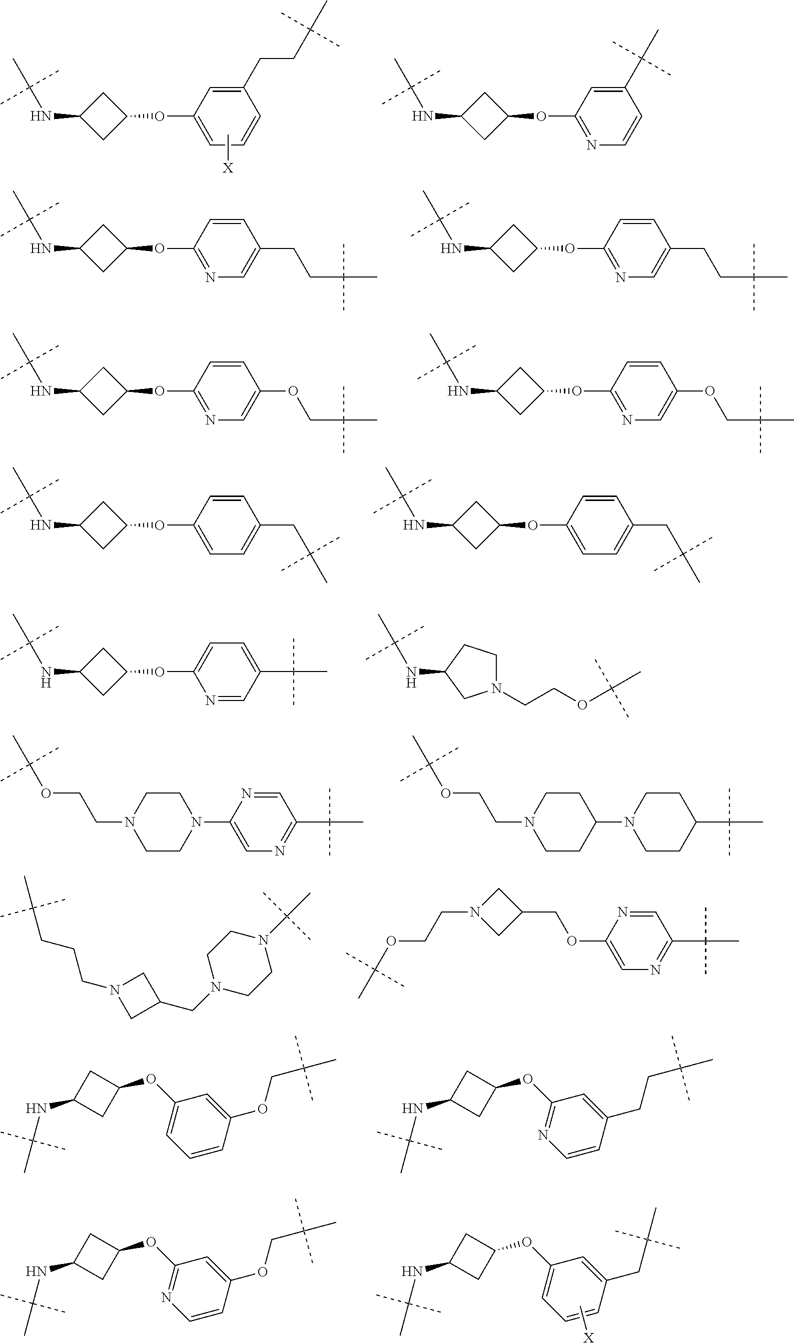

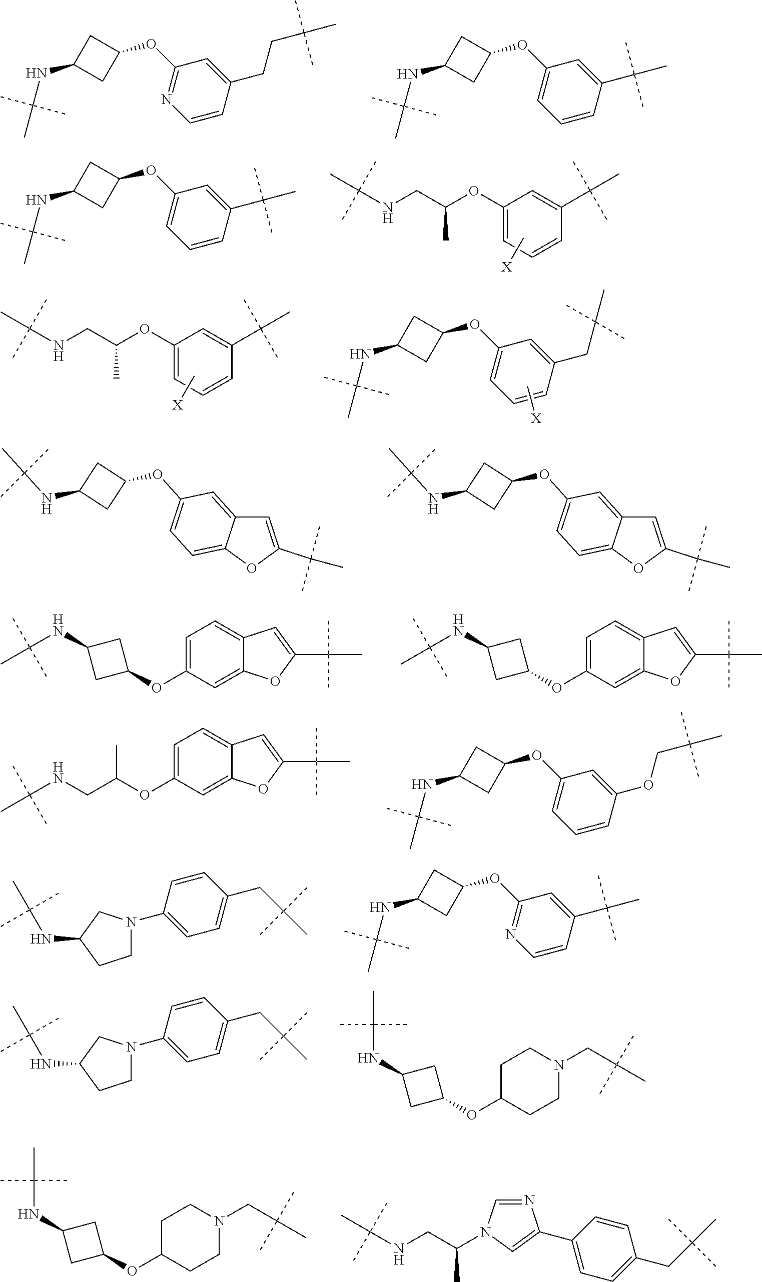

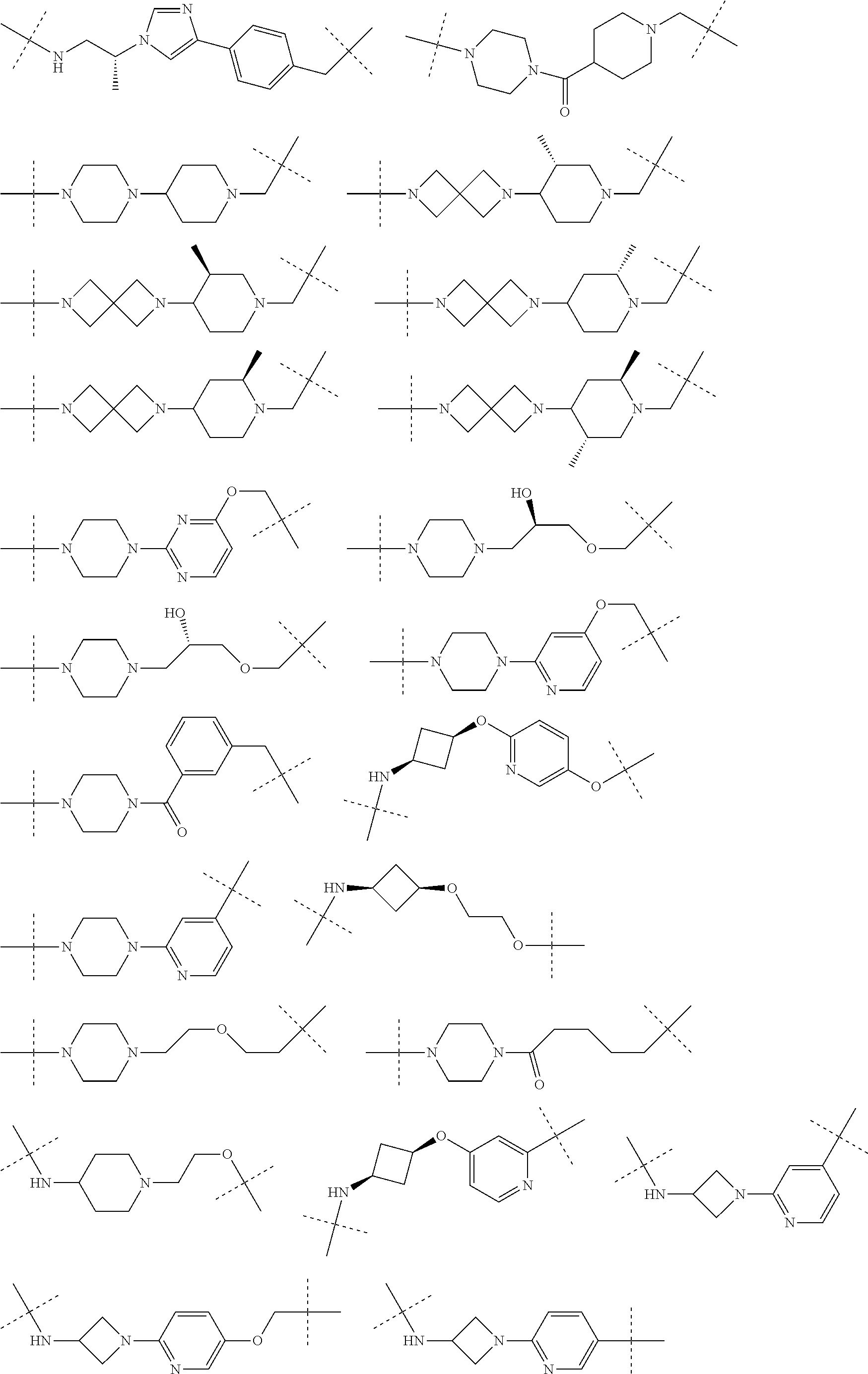

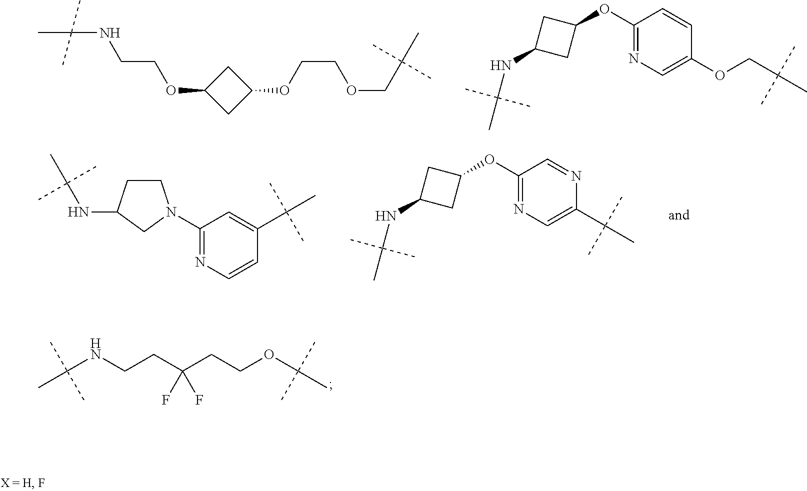

wherein n of the linker is 0, 1, 2, 3, 4, 5, 6;
X of the linker is H or F; and
each of a dashed bond and the symbol “ [symbol] ” indicate a point of attachment to the protein phosphatase ligand or the target protein ligand.

In some embodiments, n of the linker is 2, 3, 4, or 5. In some embodiments, n of the linker is 2 or 3.

In some embodiments, the linker is selected from the group consisting of:

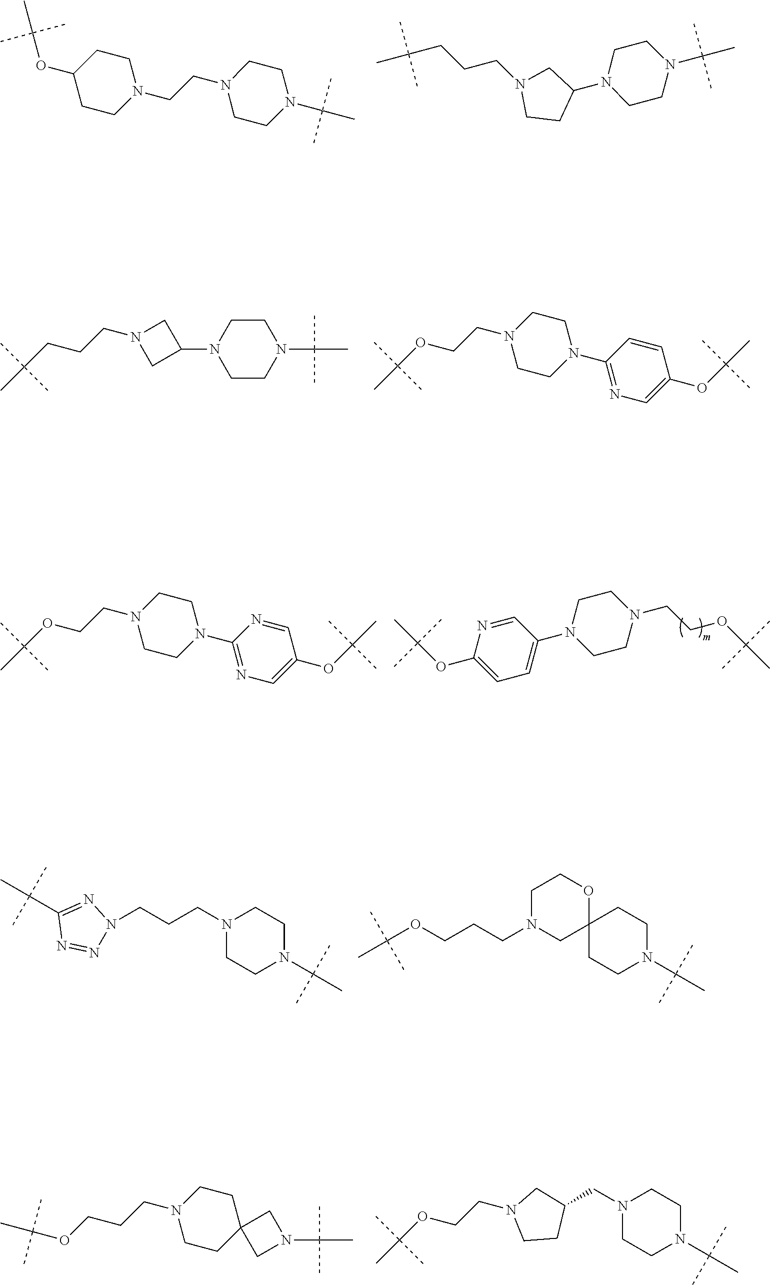

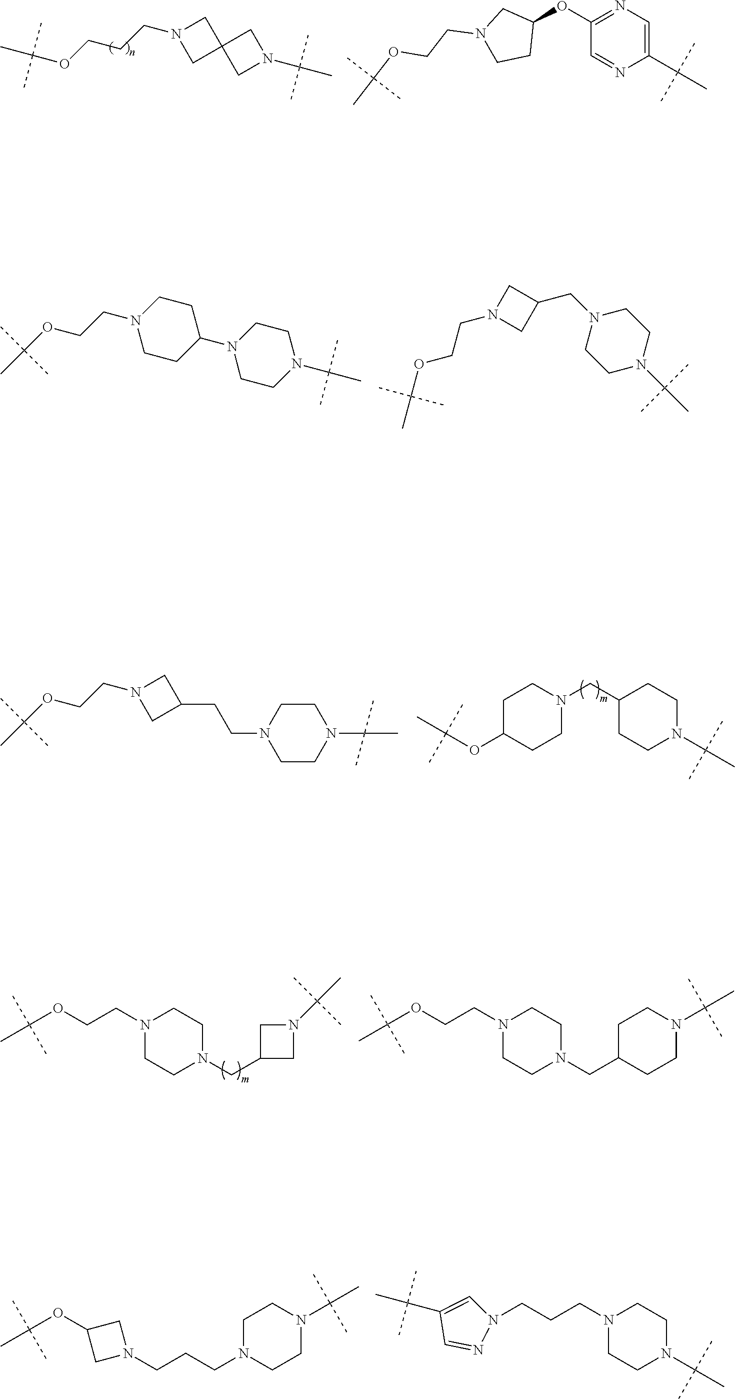

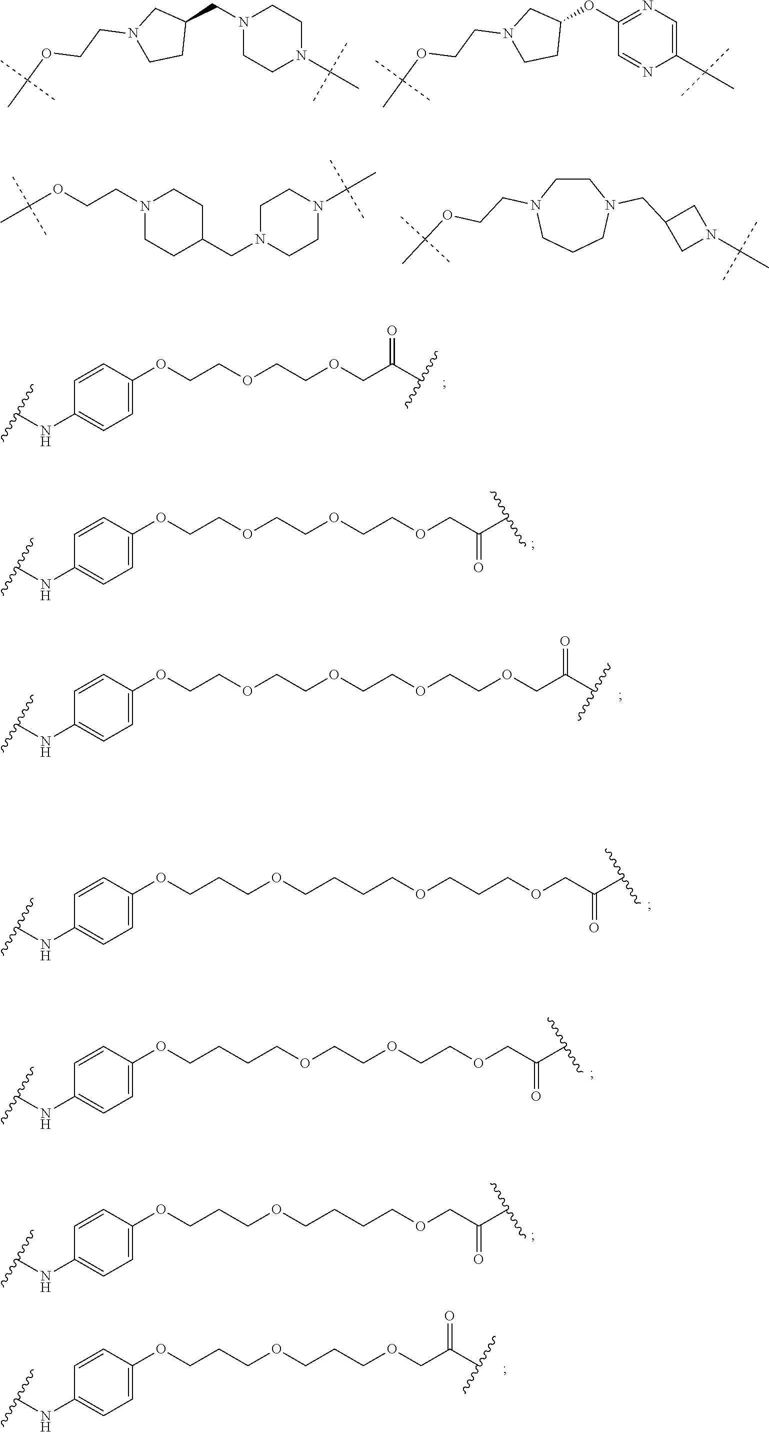

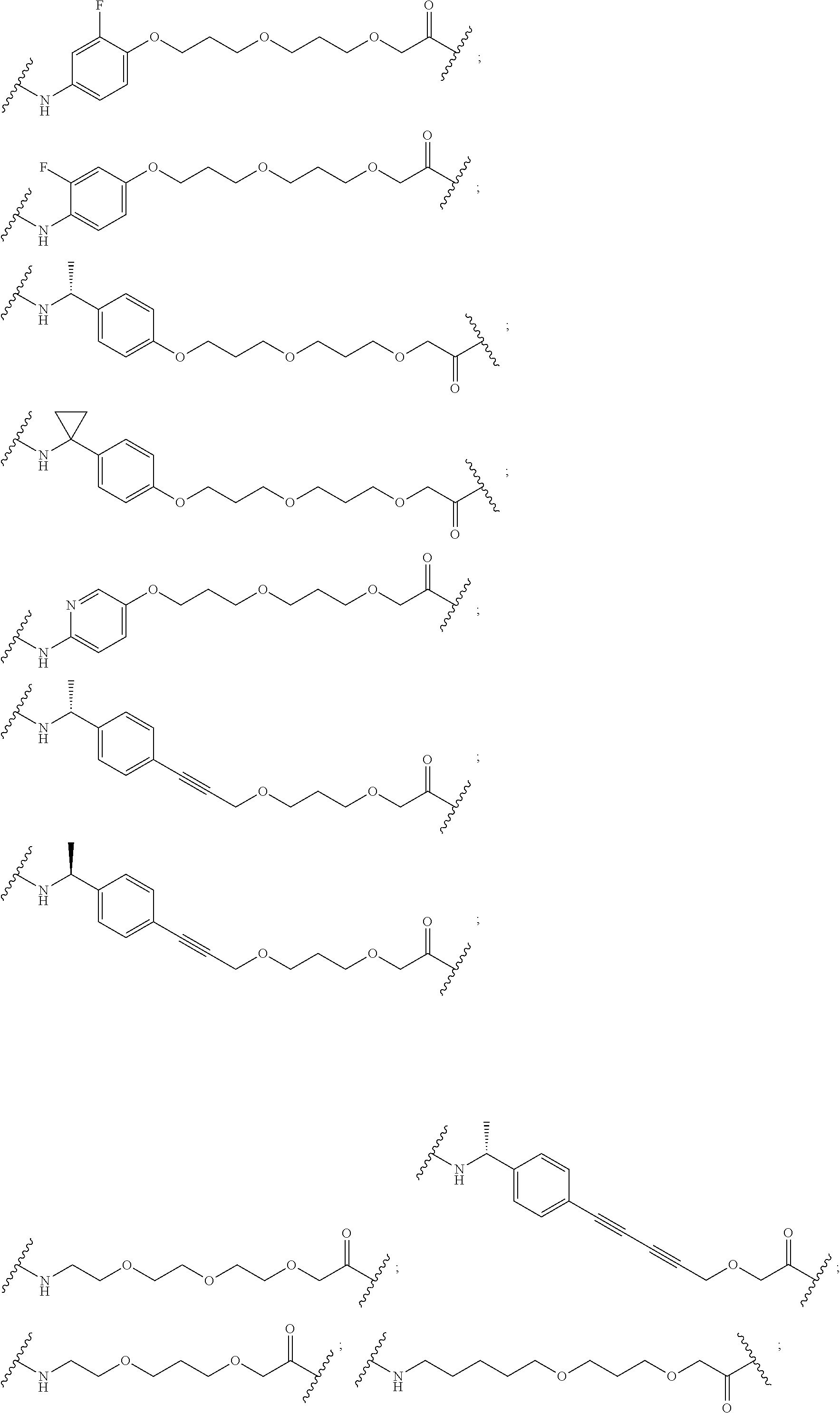

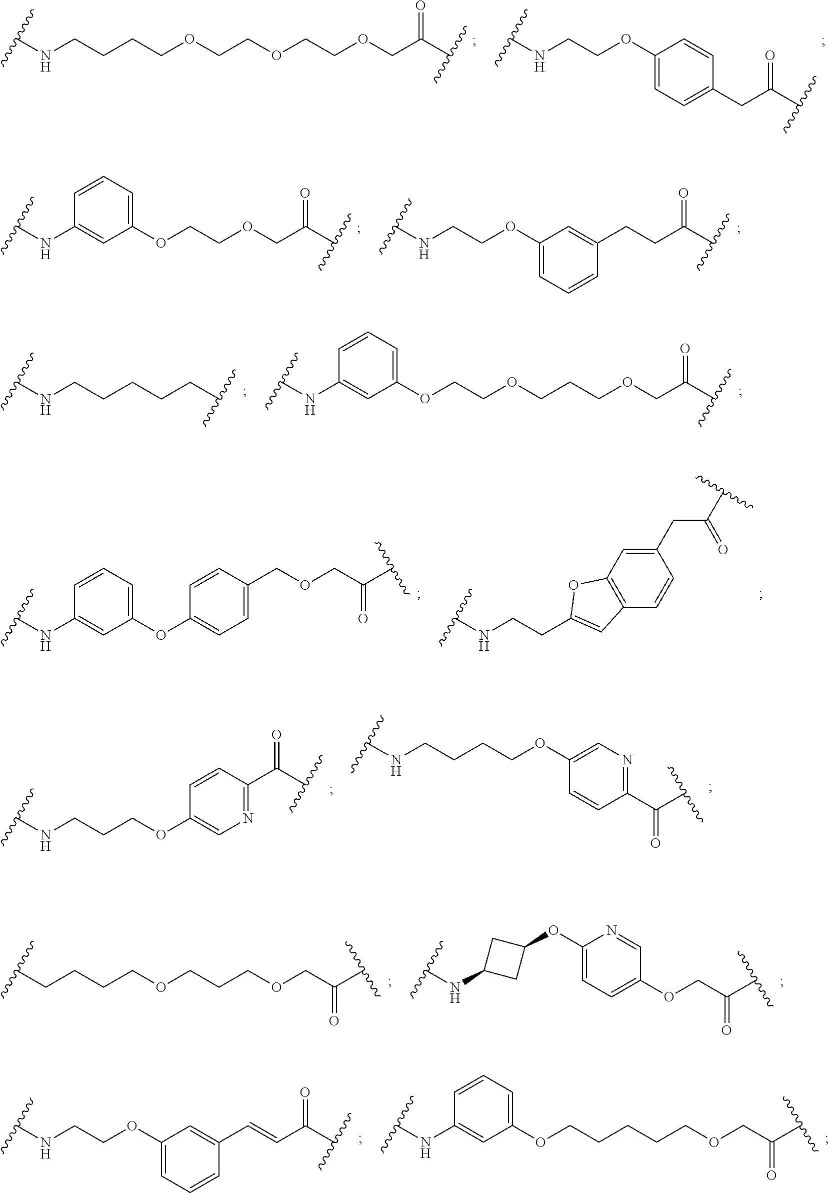

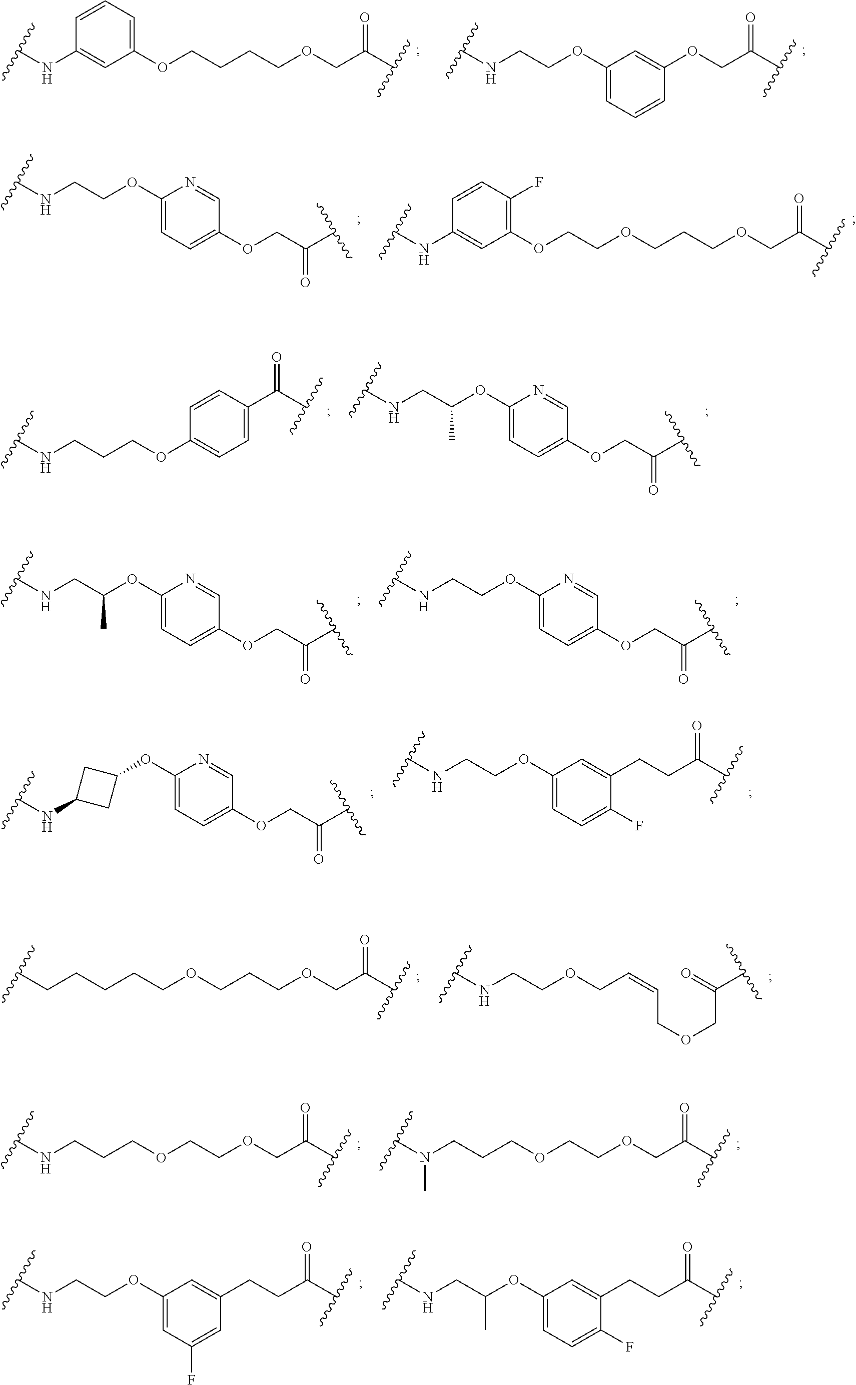

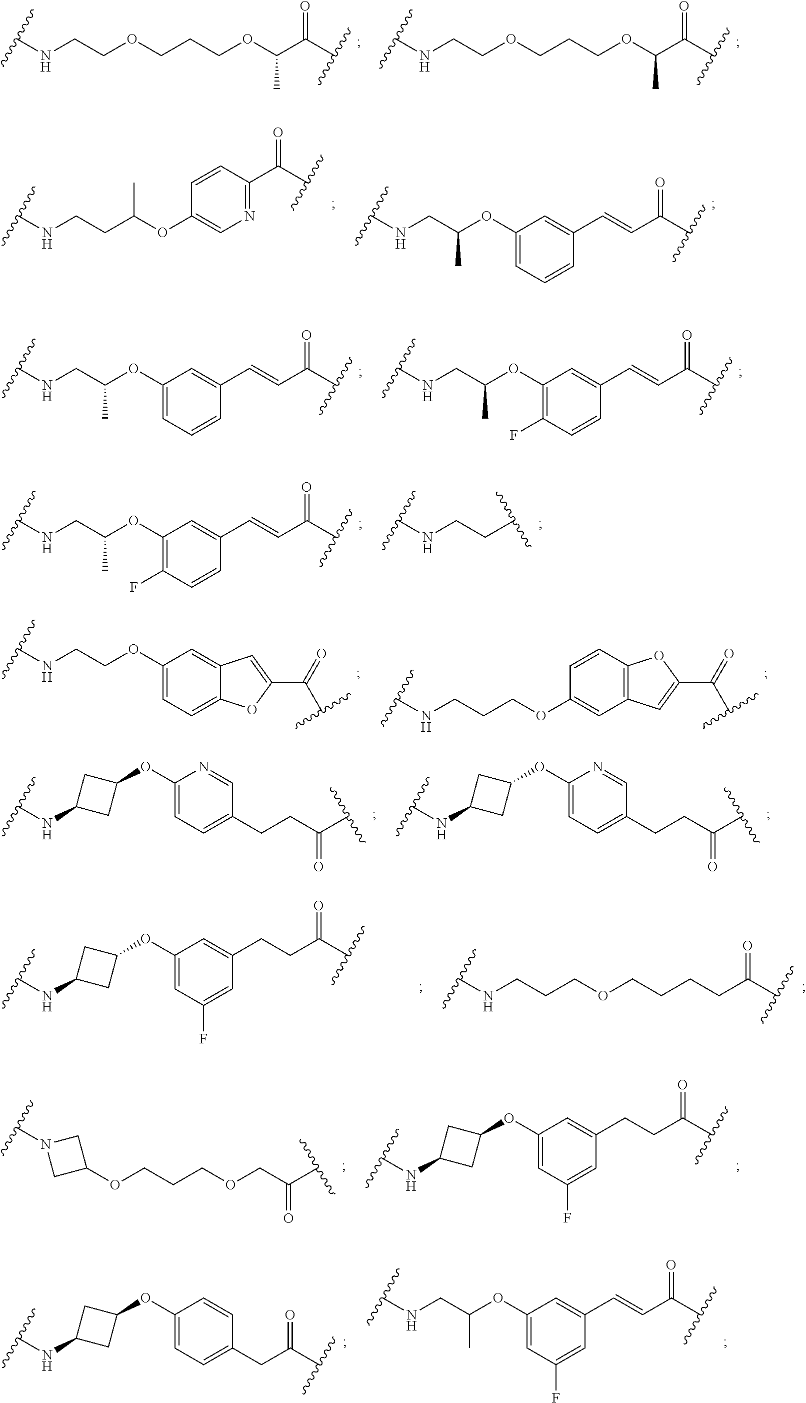

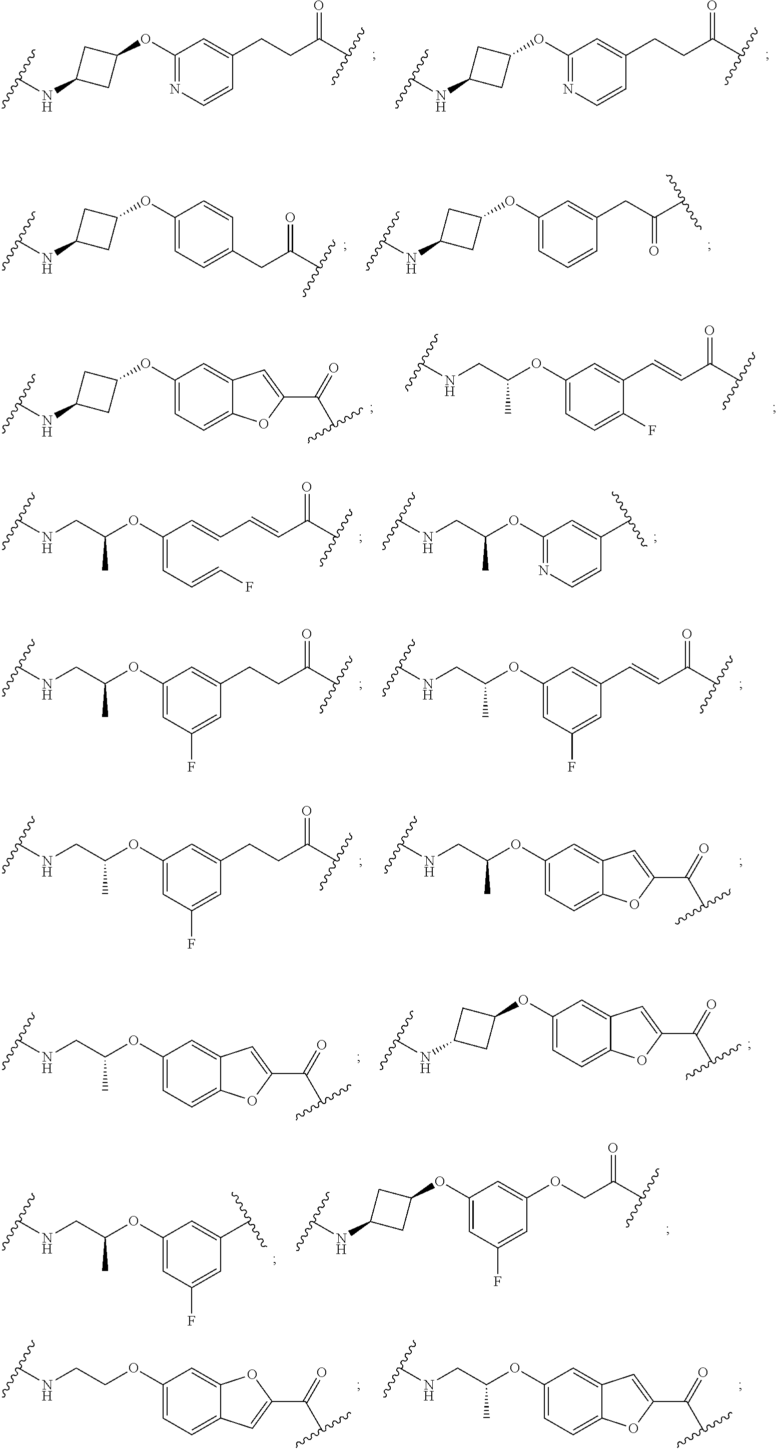

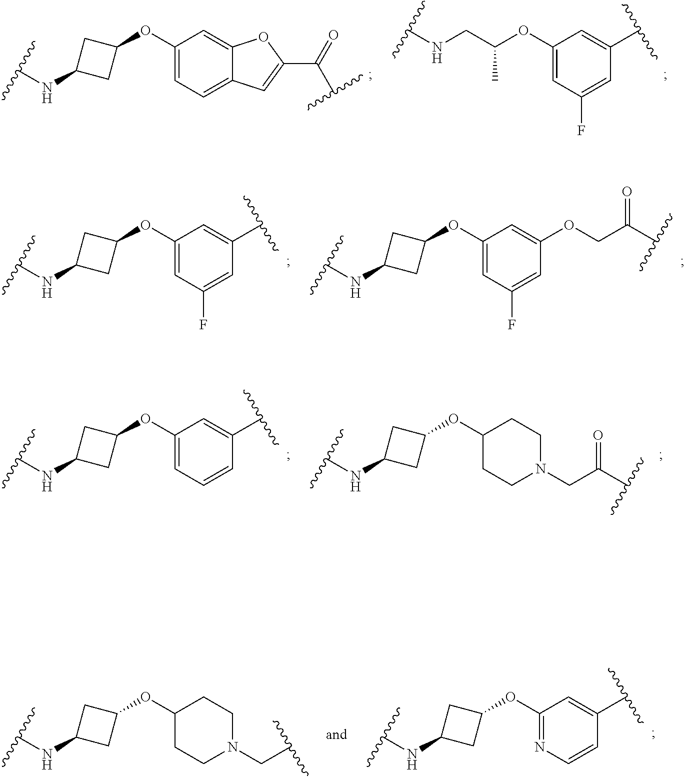

wherein each m and n in the linker is independently 0, 1, 2, 3, 4, 5, or 6;
each of a dashed bond and the symbol “ [symbol] ” indicate a point of attachment to the protein phosphatase ligand or the target protein ligand.

In certain embodiments, the linker is a linear chain with from 4 to 24 linear atoms, and the carbon atom in the linear chain can be replaced with oxygen, nitrogen, amide, fluorine, or other atom. For example, in some embodiments, the linker is one of the following:

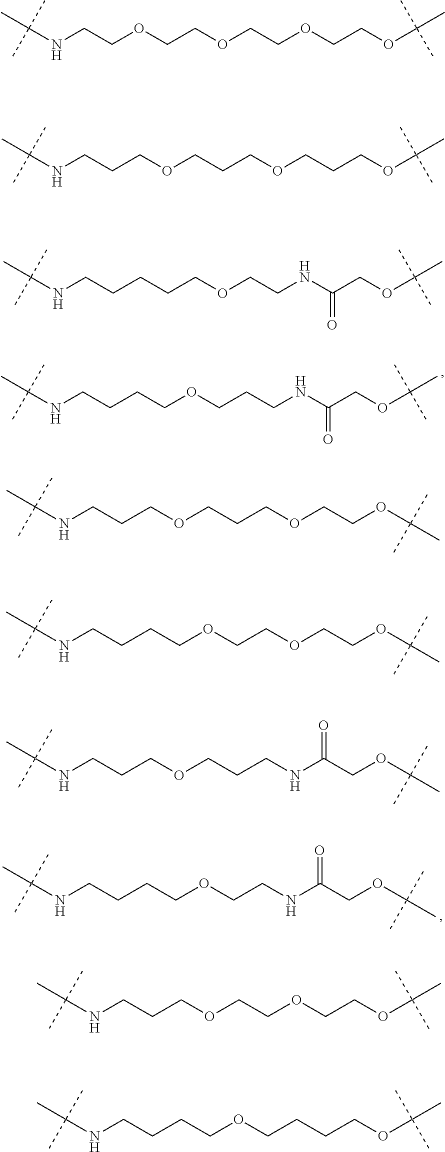

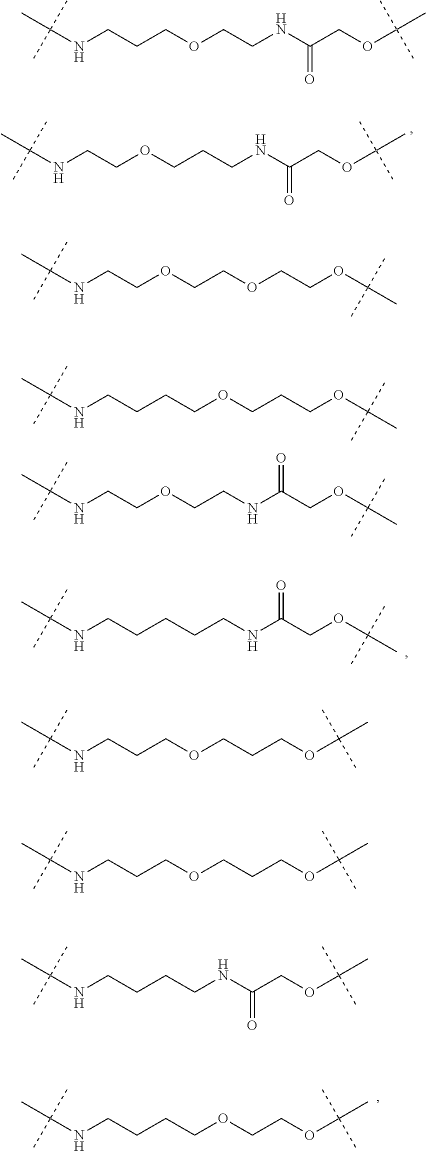

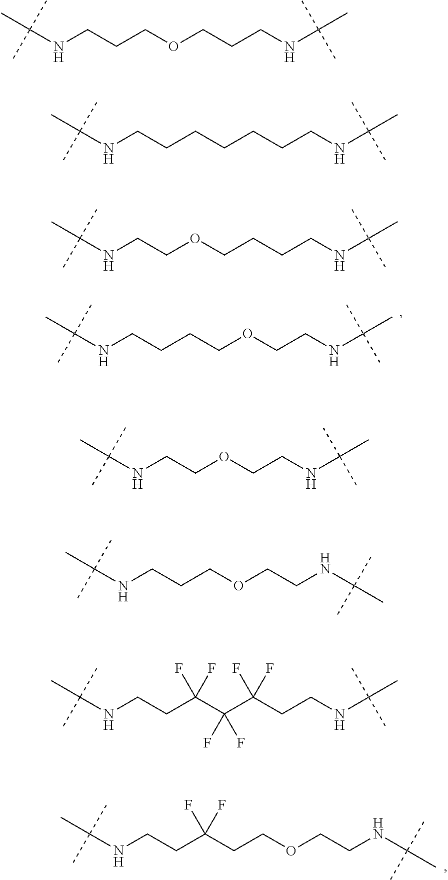

wherein a dashed bond indicates a point of attachment to the protein phosphatase ligand or the target protein ligand.

In certain embodiments, the linker includes one or more cyclic groups, such as aliphatic, aromatic, or heteroaromatic cyclic moieties. For example, in some embodiments, the linker is one of the following:

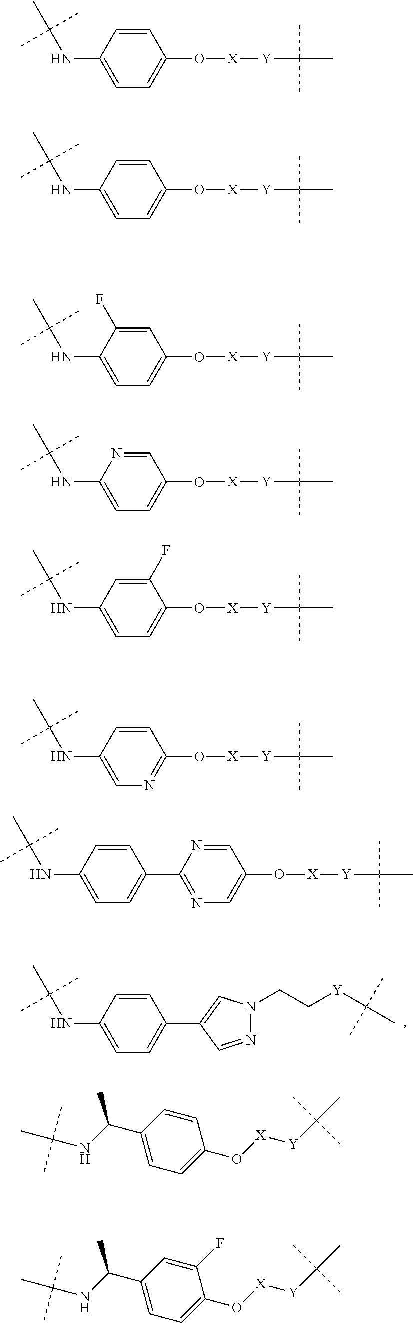

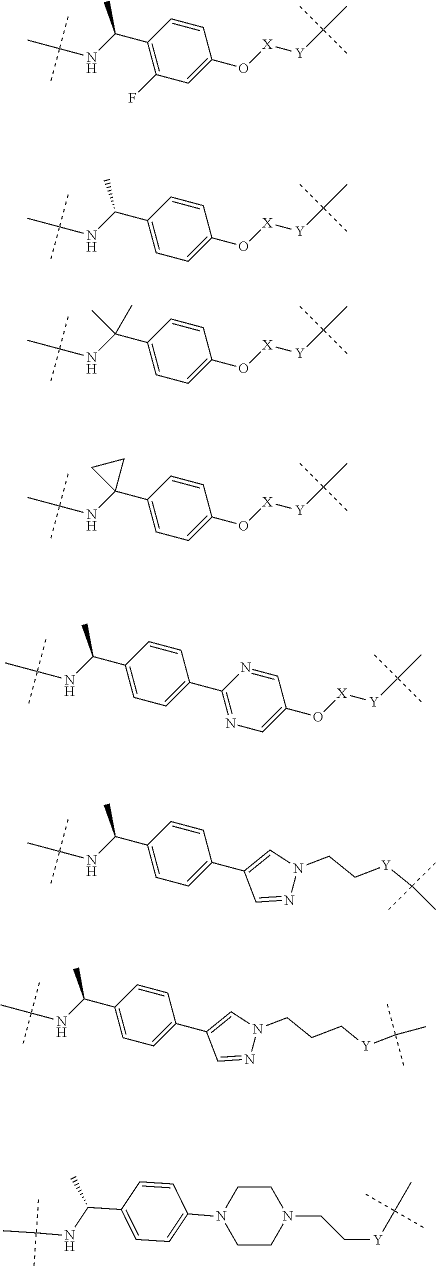

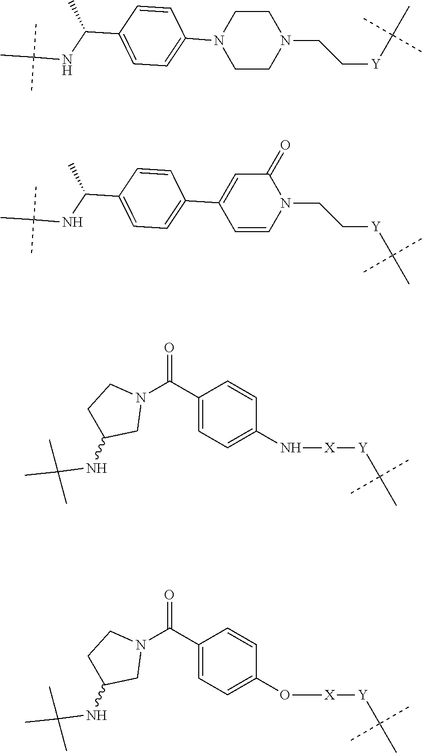

wherein:
X in the linker is a linear chain of 2 to 14 atoms, optionally including one or more heteroatoms (e.g., an C₂₋₁₄ alkylene or 2-14 membered heteroalkylene);
Y is O, N, or S(O)ₙ;
n in the linker is 0, 1, or 2; and
a dashed bond indicates a point of attachment to the protein phosphatase ligand or the target protein ligand.

In some embodiments, the linker is one of the following:

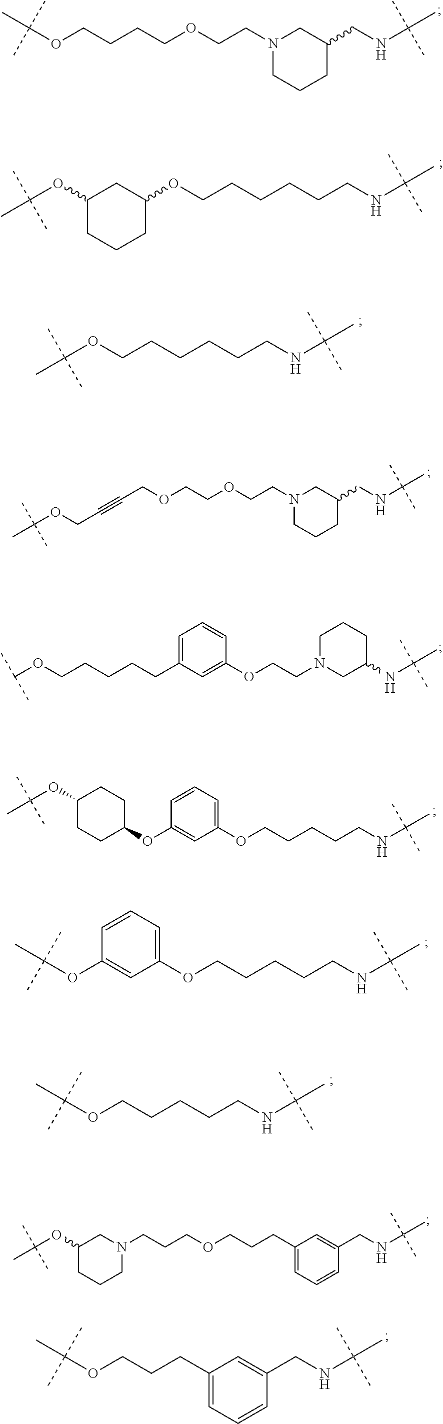

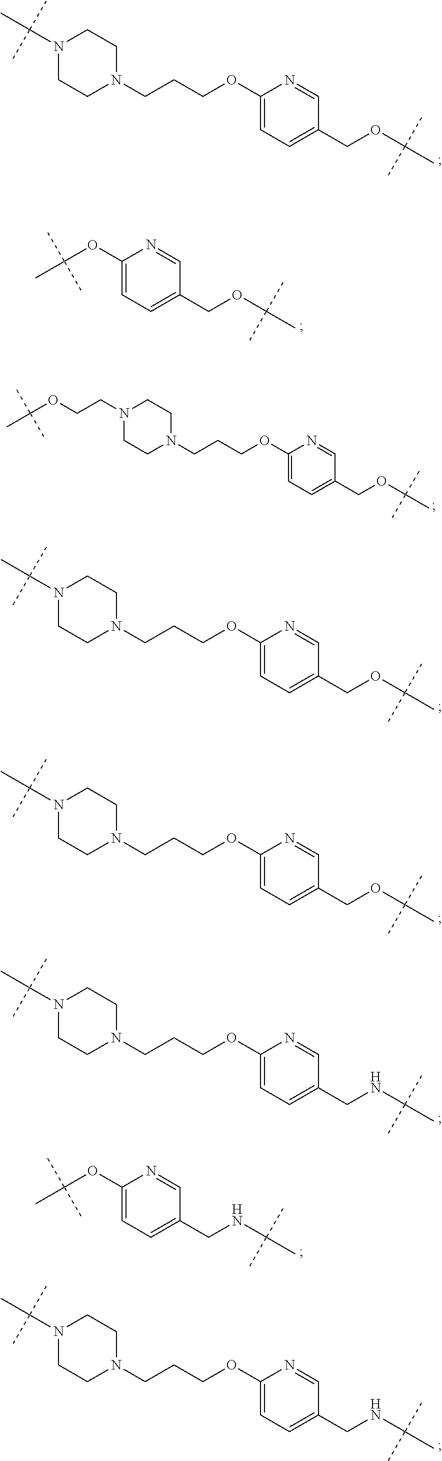

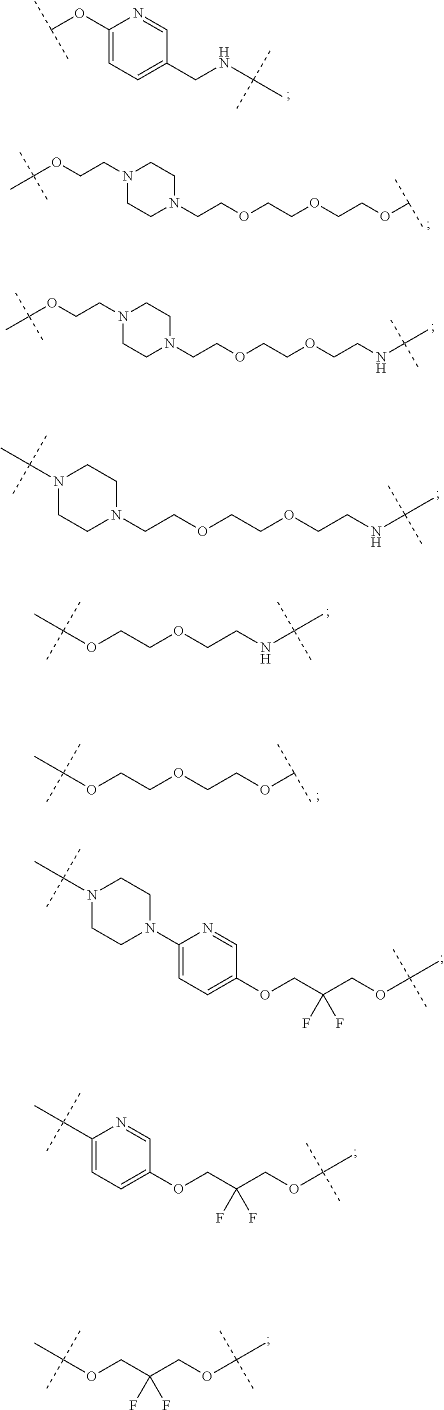

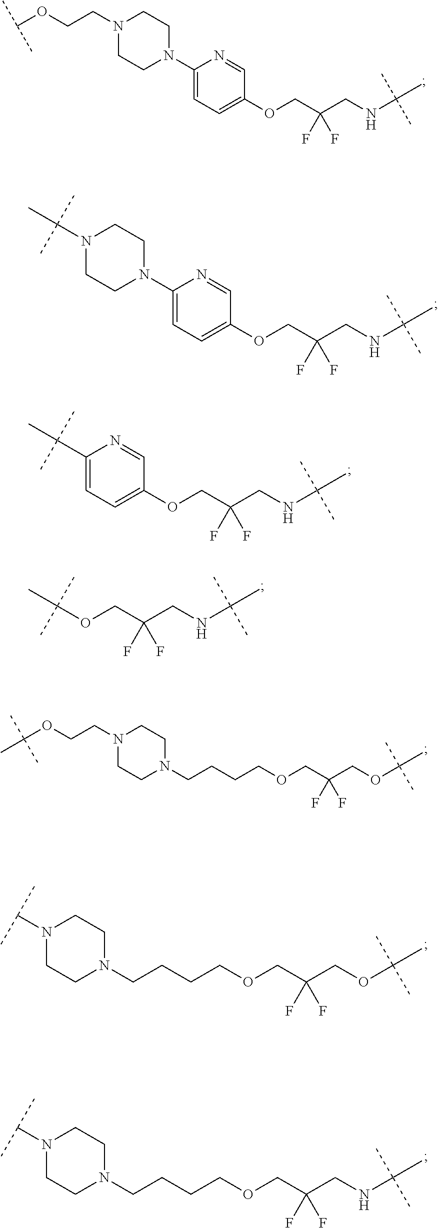

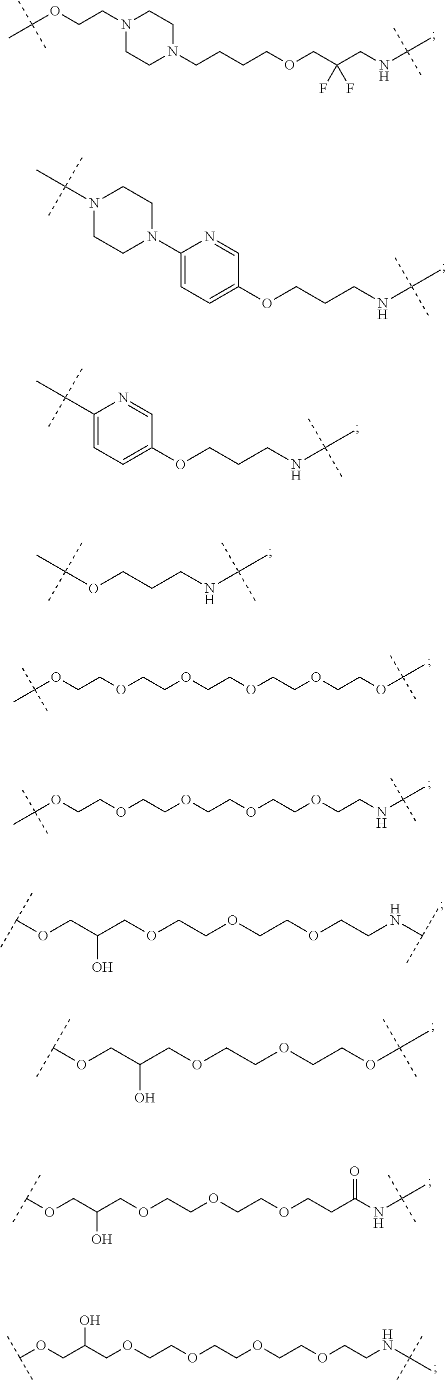

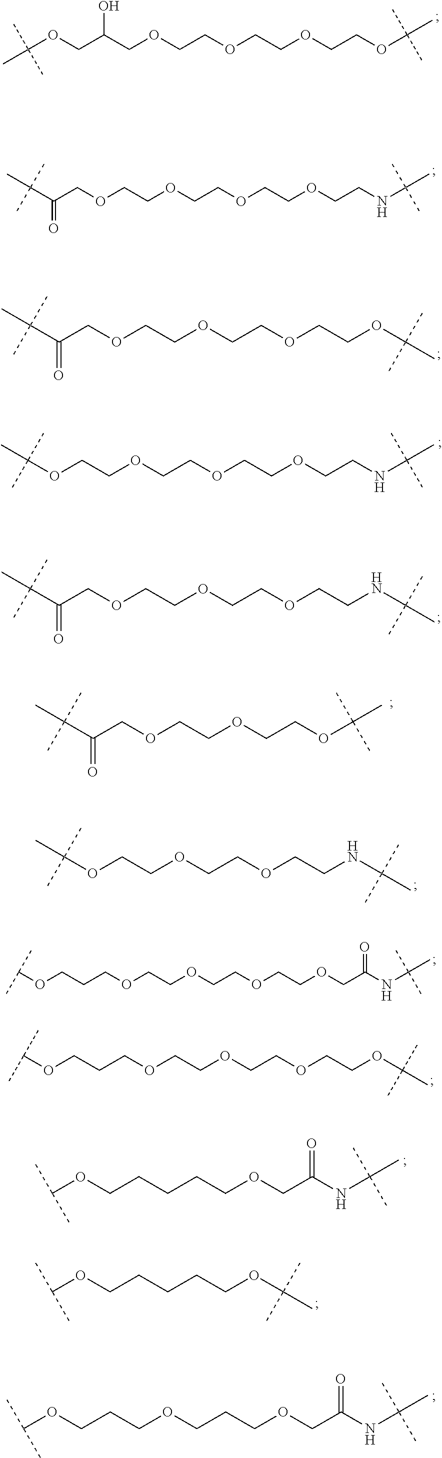

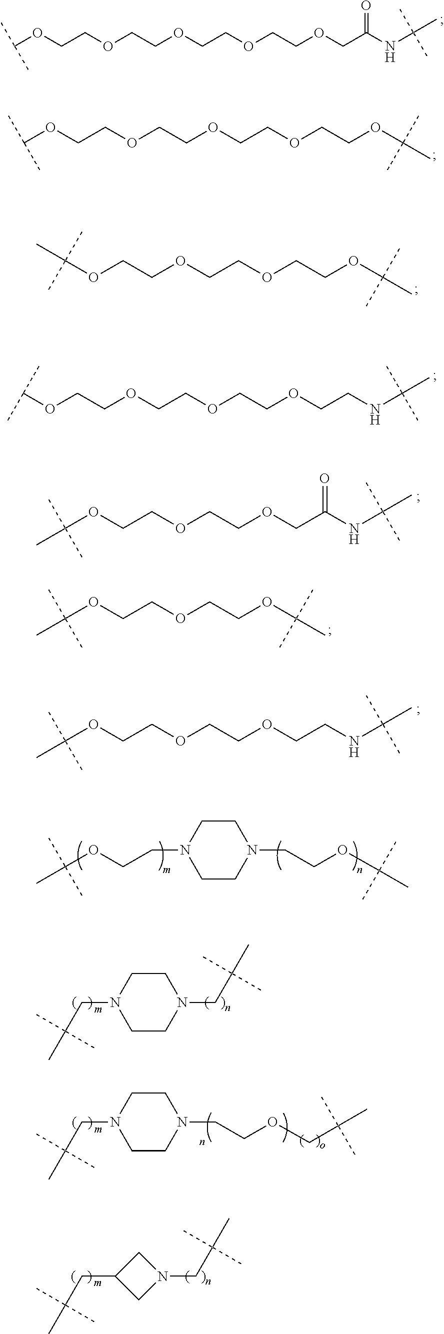

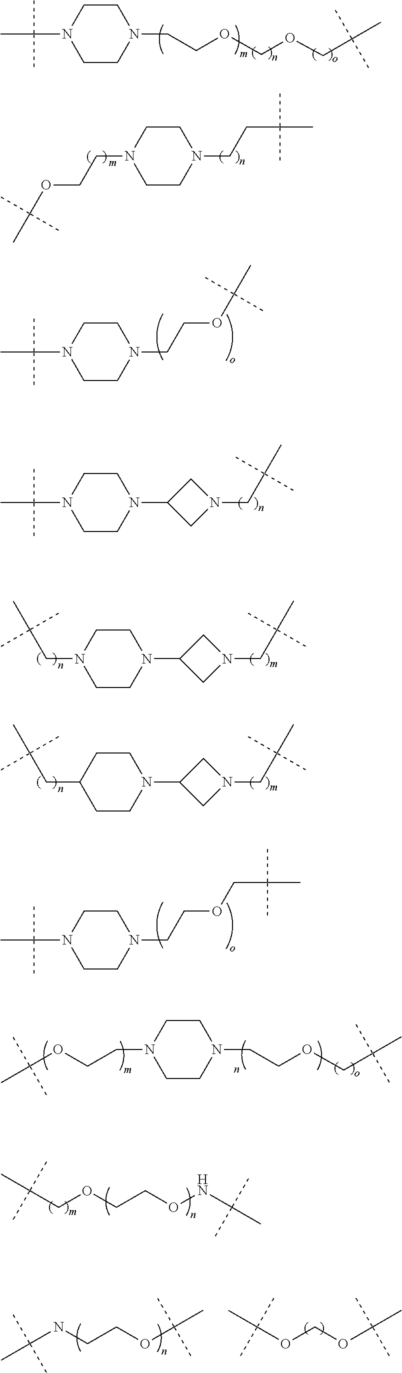

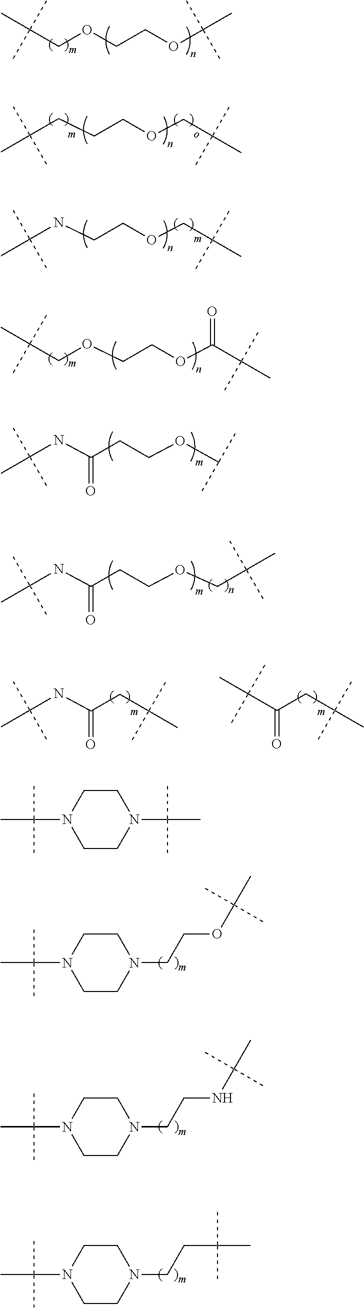

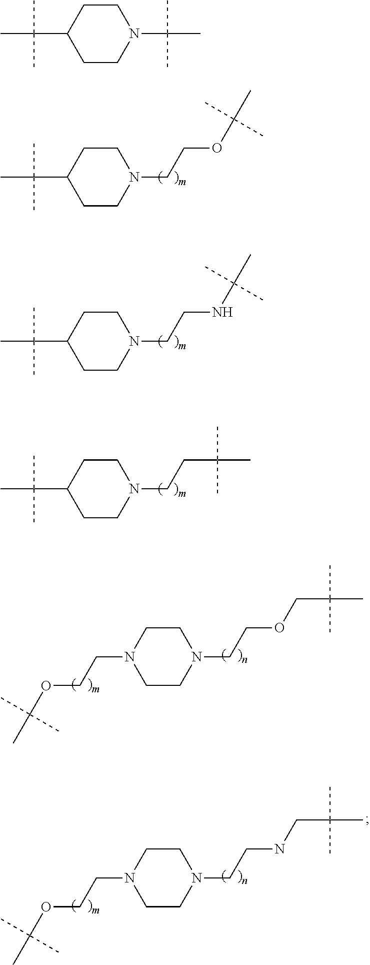

wherein each m, n, and o in the linker is independently 0, 1, 2, 3, 4, 5, 6, 7, 8, 9, 10, 11, 12, 13, 14, 15, 16, 17, 18, 19, or 20; and a dashed bond indicates a point of attachment to the protein phosphatase ligand or the target protein ligand.

In some embodiments, the linker is selected from the group consisting of:

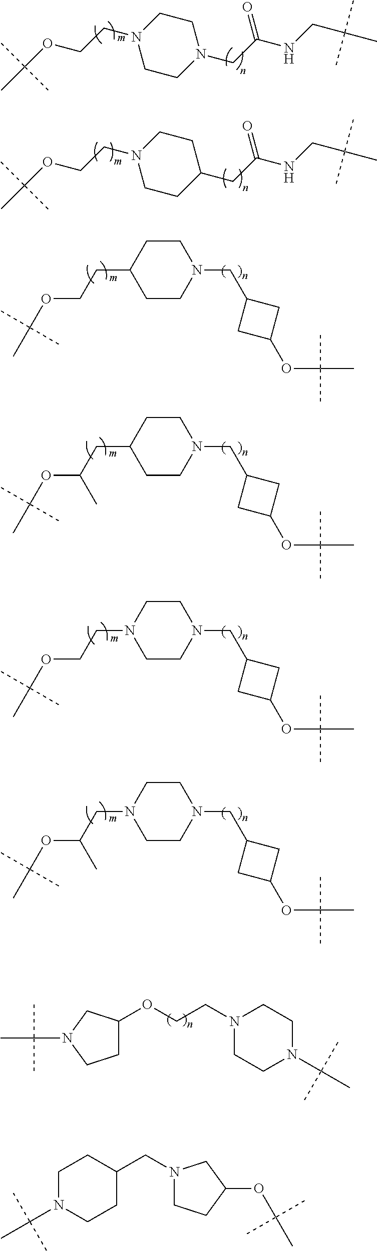

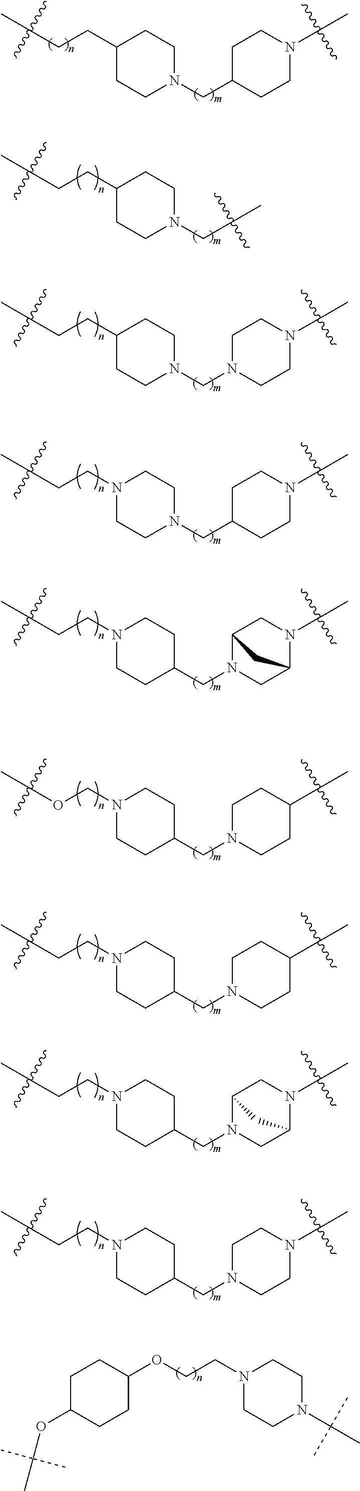

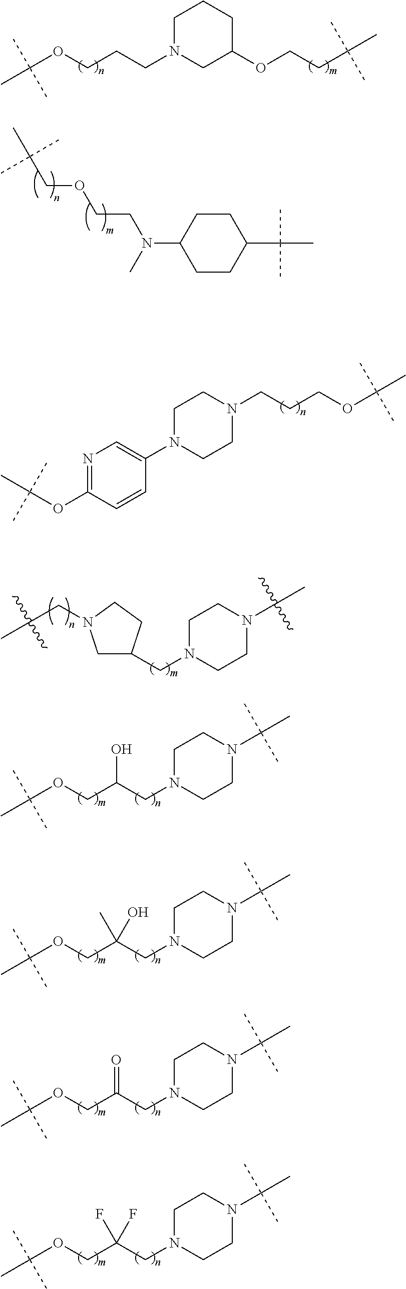

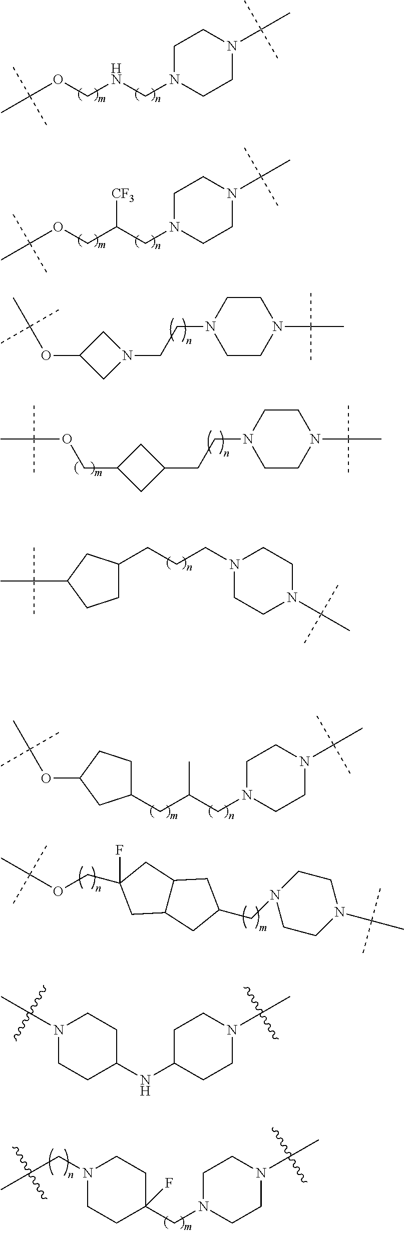

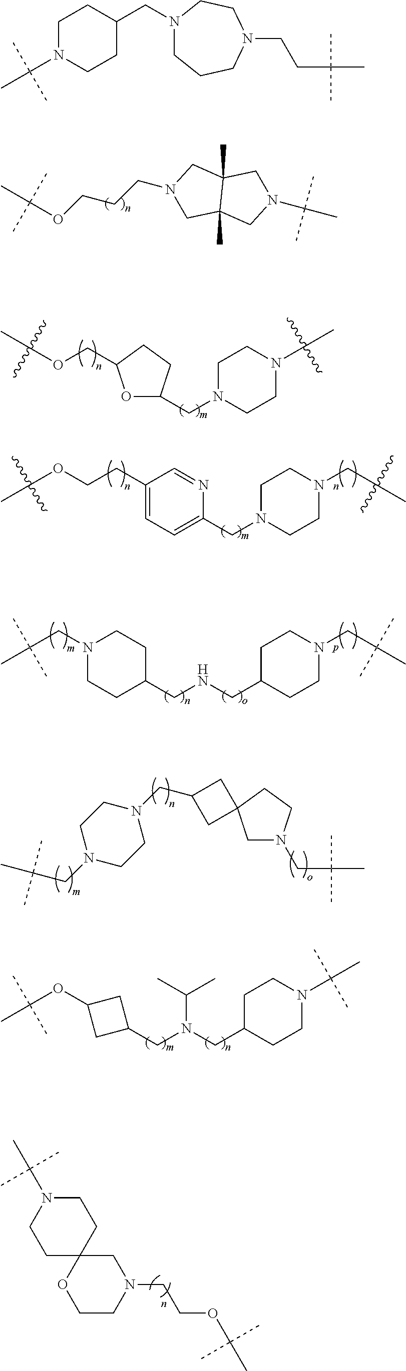

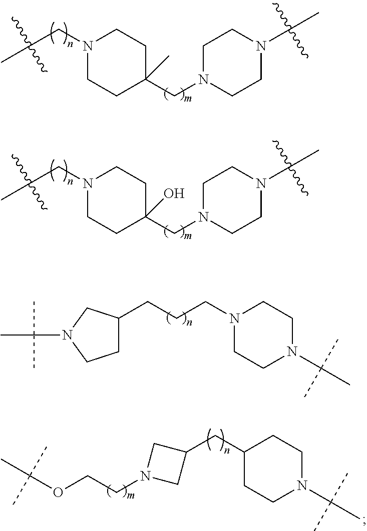

wherein each m, n, o, and p in the linker is independently 0, 1, 2, 3, 4, 5, 6, 7, 8, 9, 10, 11, 12, 13, 14, 15, 16, 17, 18, 19, or 20; and a dashed bond indicates a point of attachment to the protein phosphatase ligand or the target protein ligand.

In some embodiments, the linker is selected from the group consisting of:

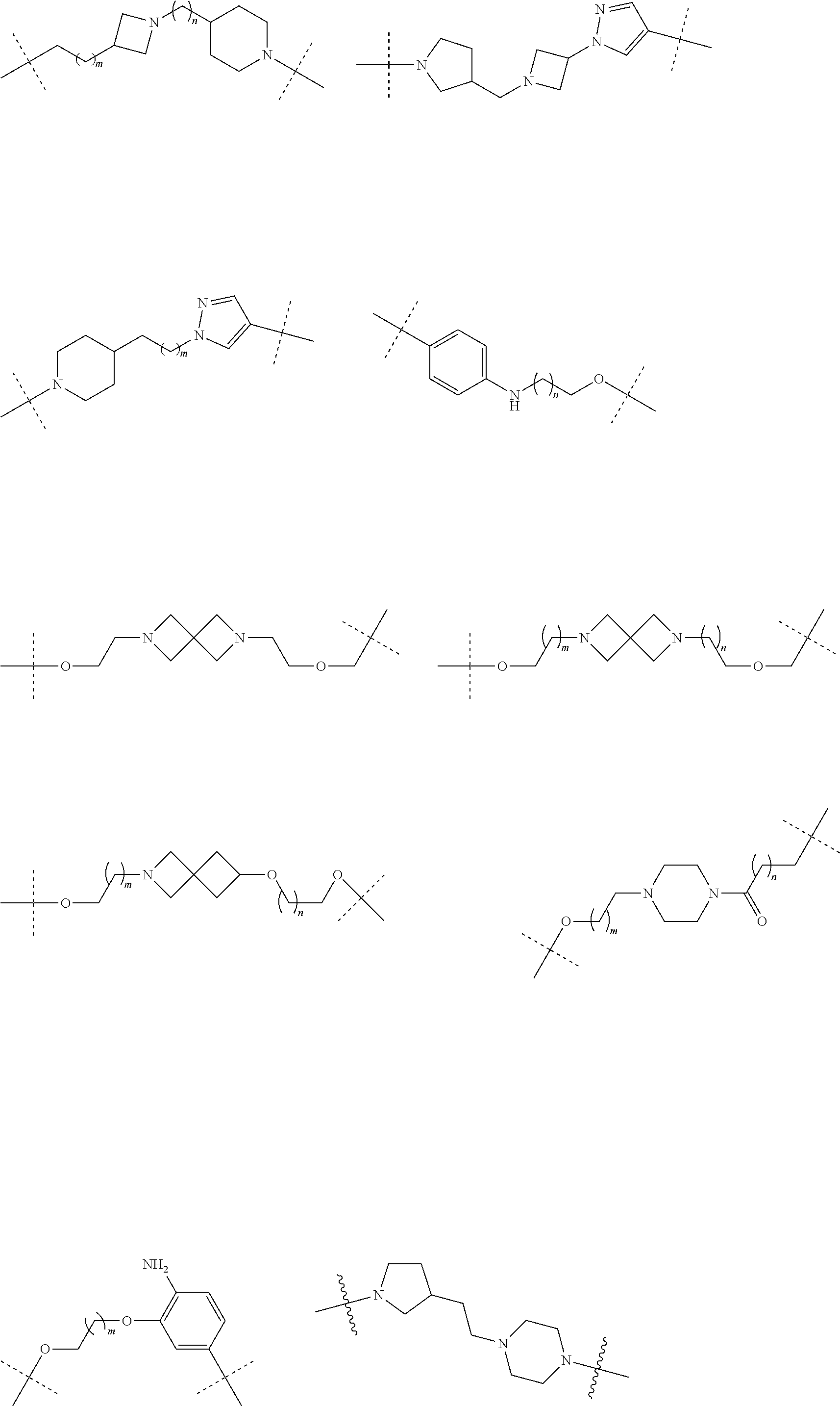

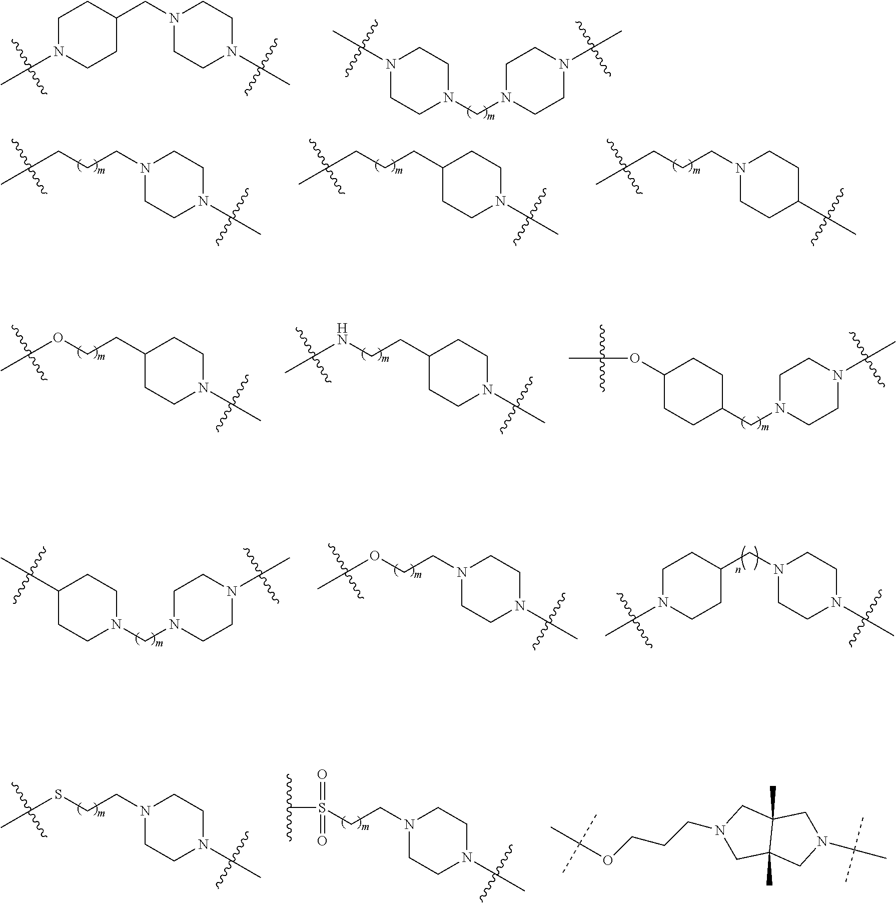

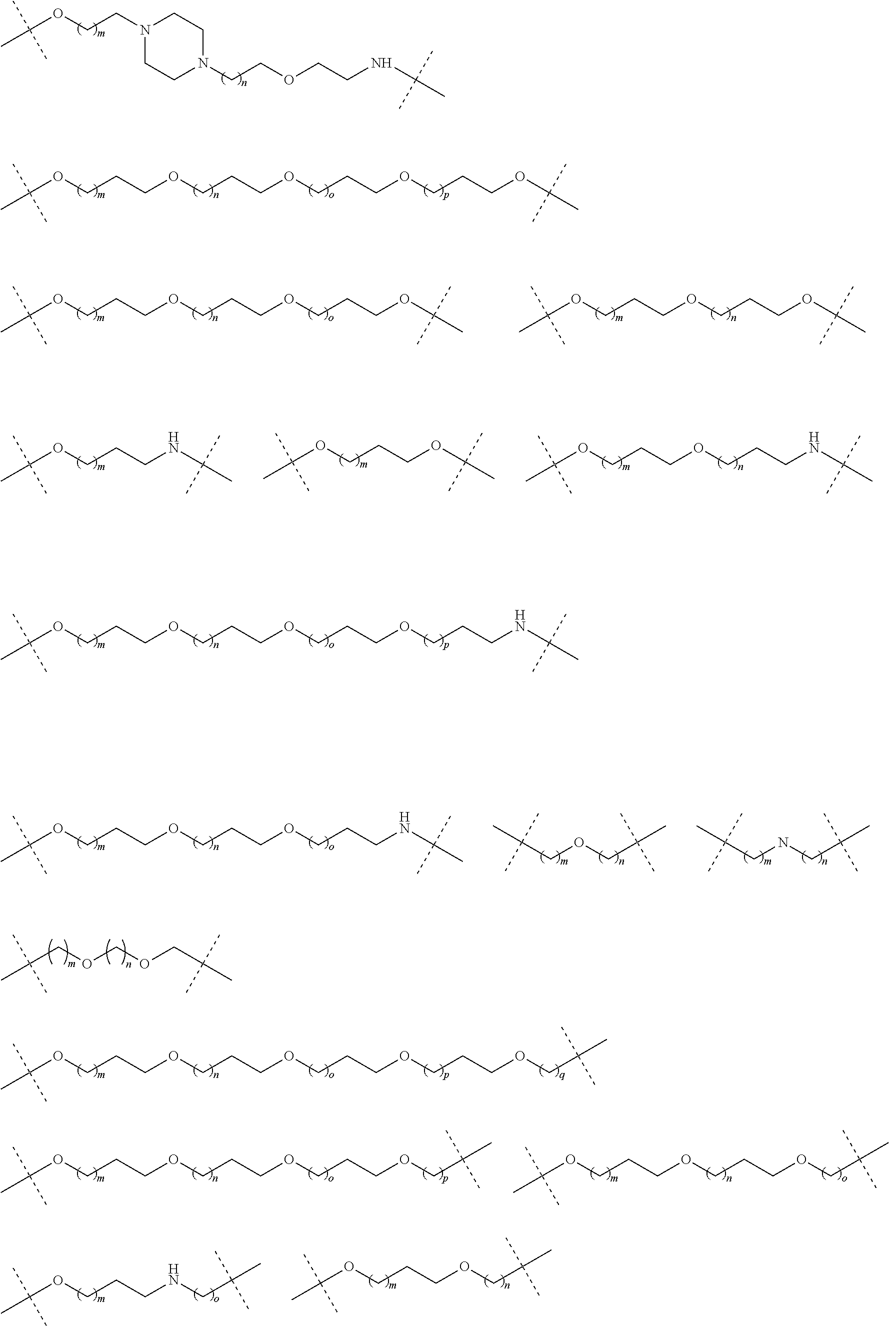

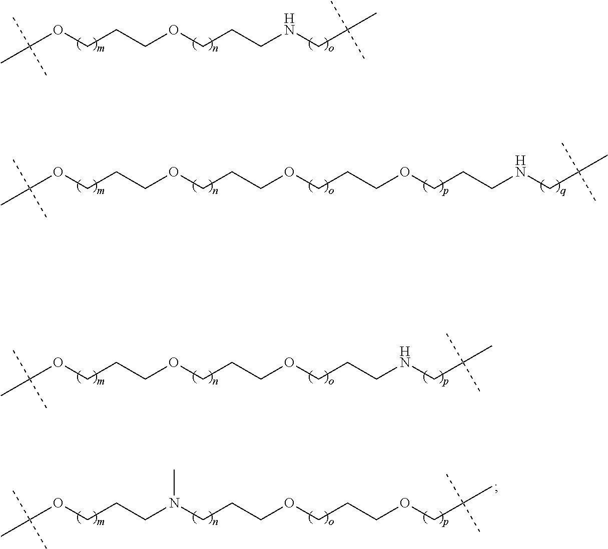

wherein each m, n, o, p, and q in the linker is independently 0, 1, 2, 3, 4, 5, 6, 7, 8, 9, 10, 11, 12, 13, 14, 15, 16, 17, 18, 19, or 20; and each of a dashed bond and the symbol “ [symbol] ” indicate a point of attachment to the protein phosphatase ligand or the target protein ligand.

In some embodiments, the linker is one of the following:

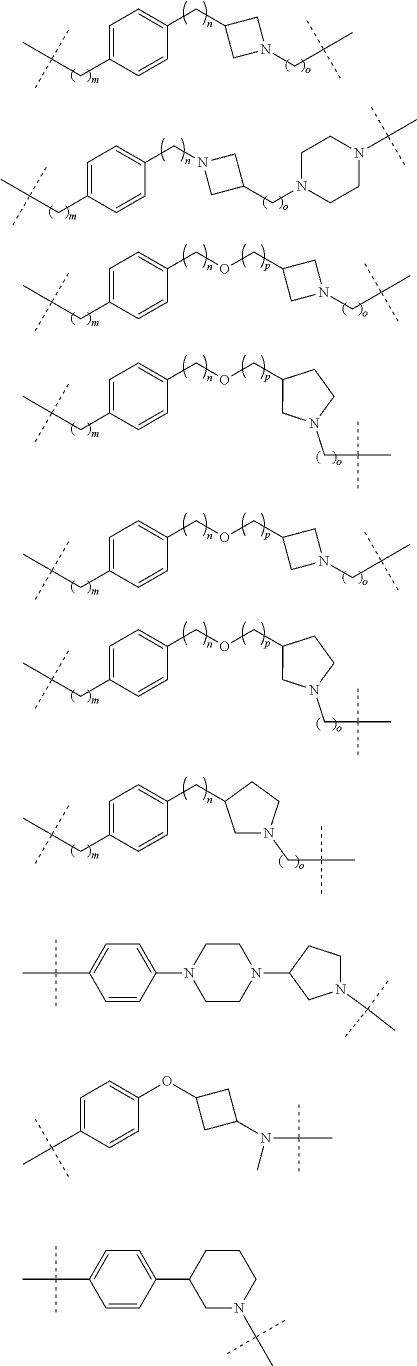

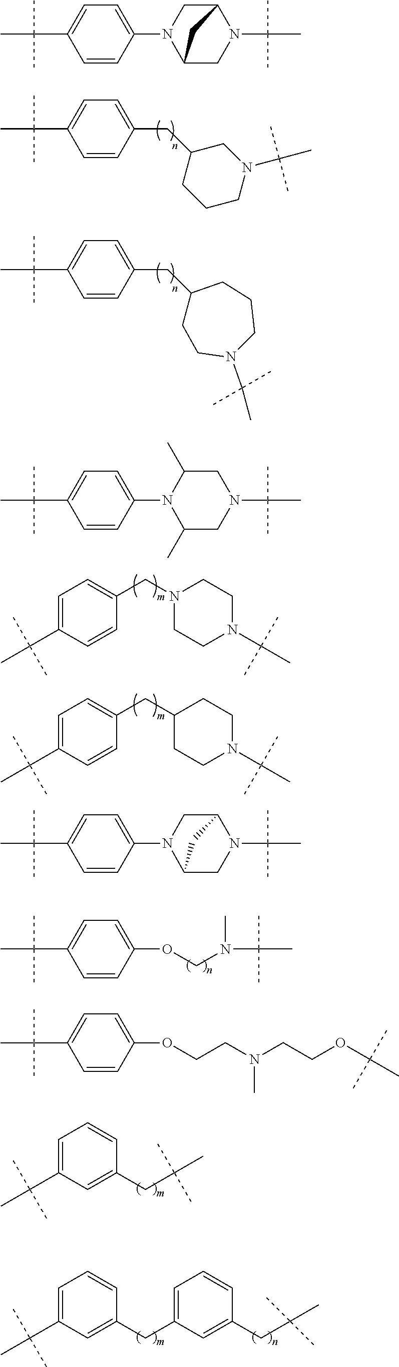

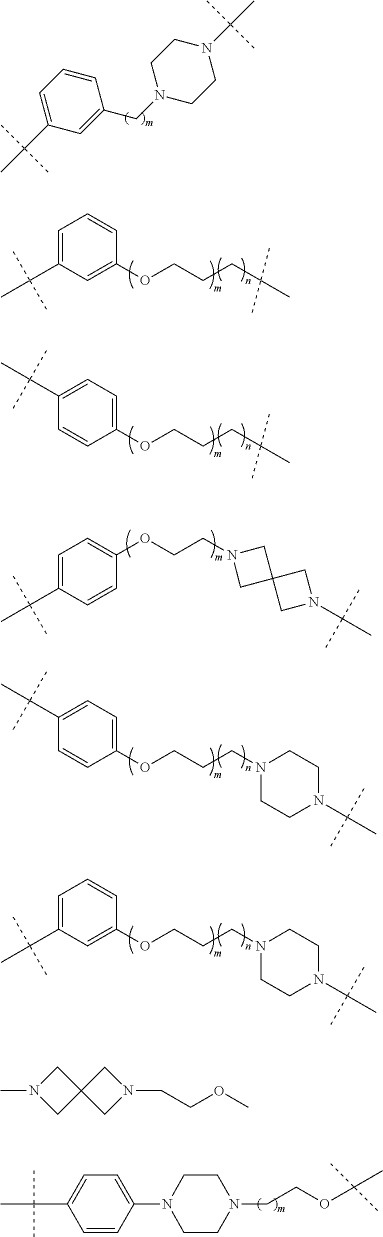

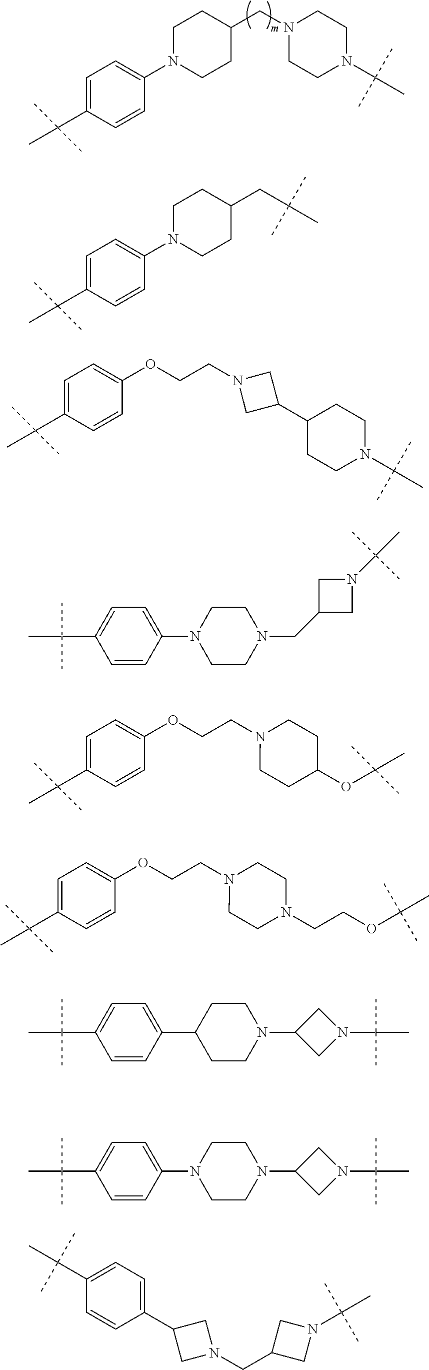

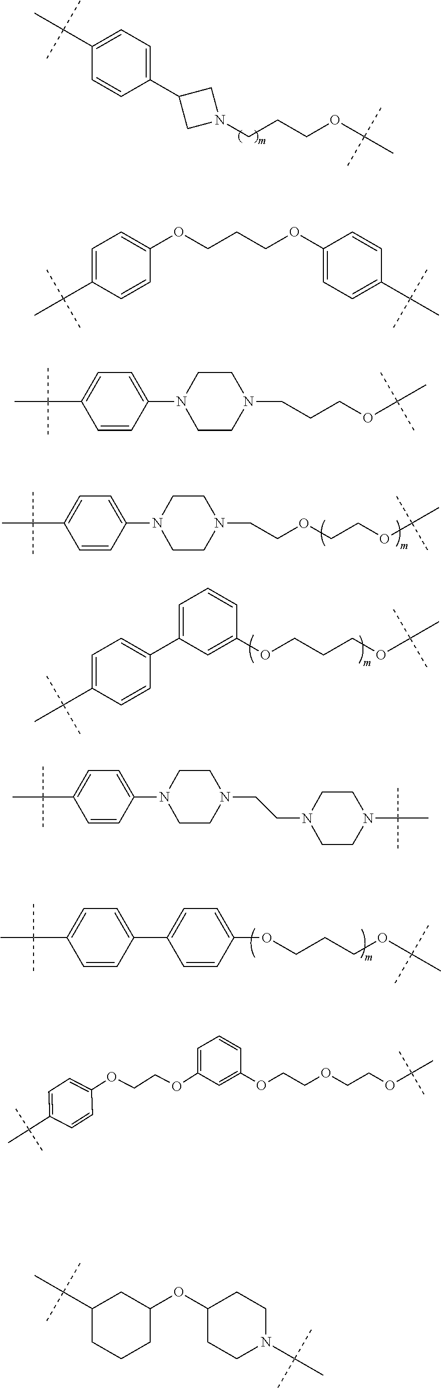

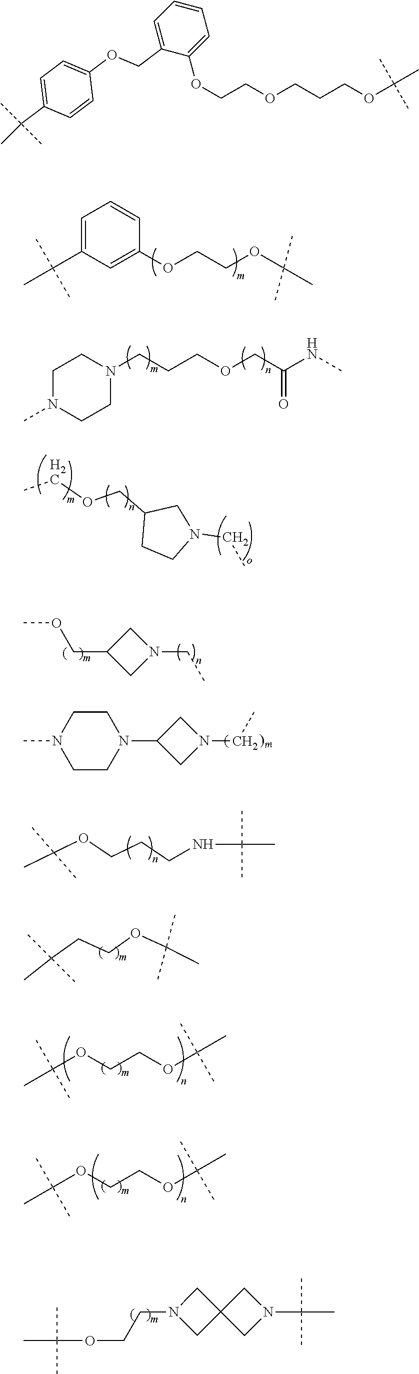

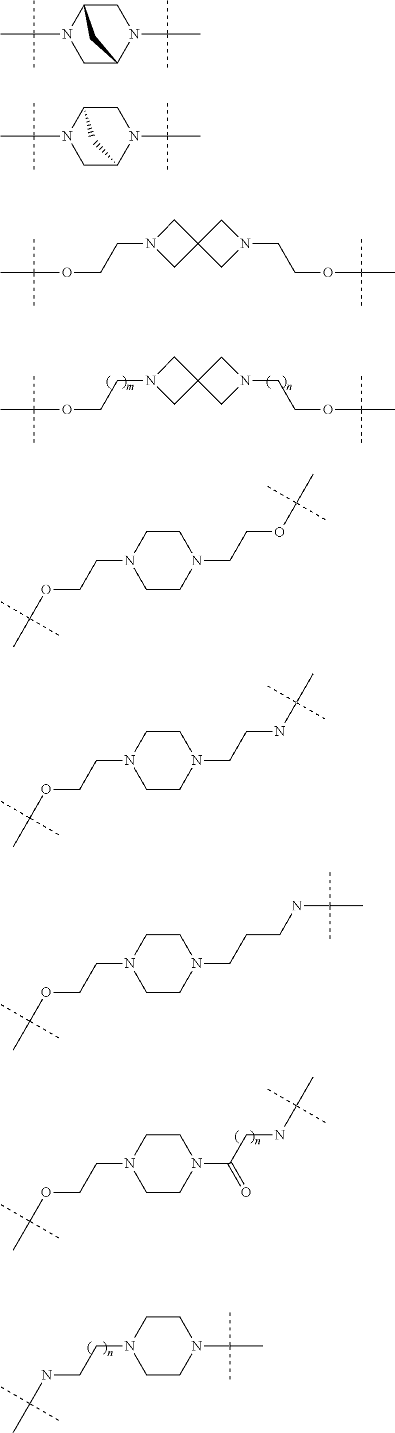

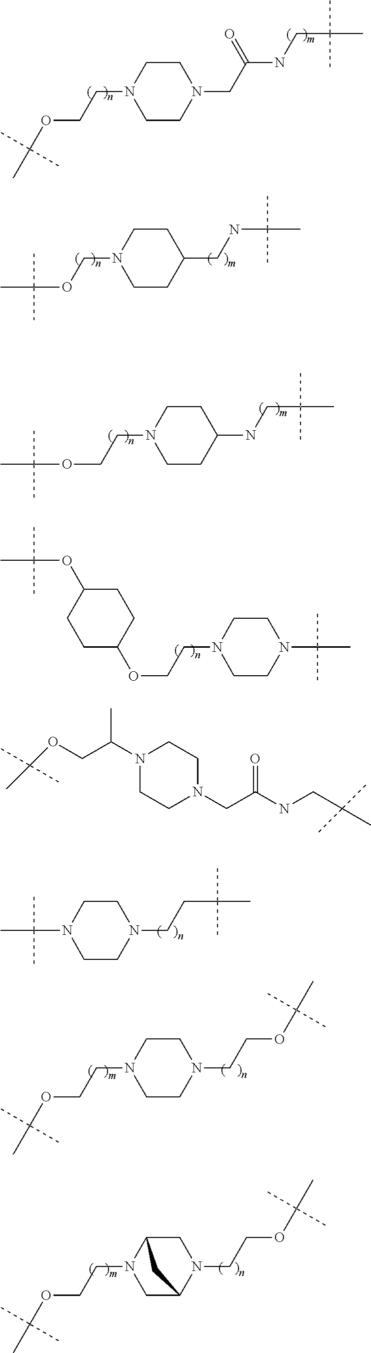

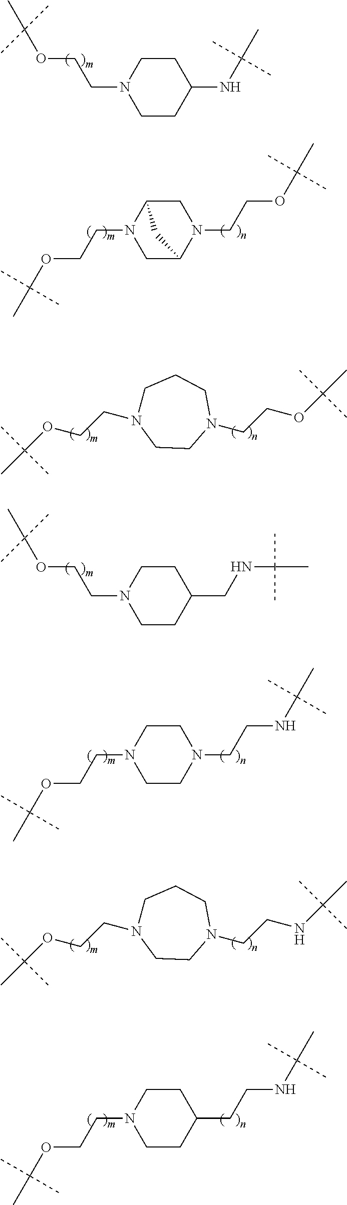

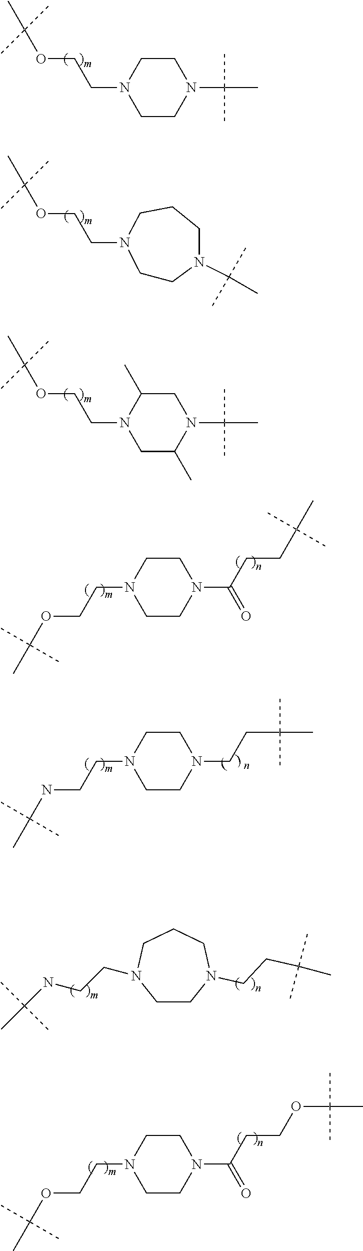

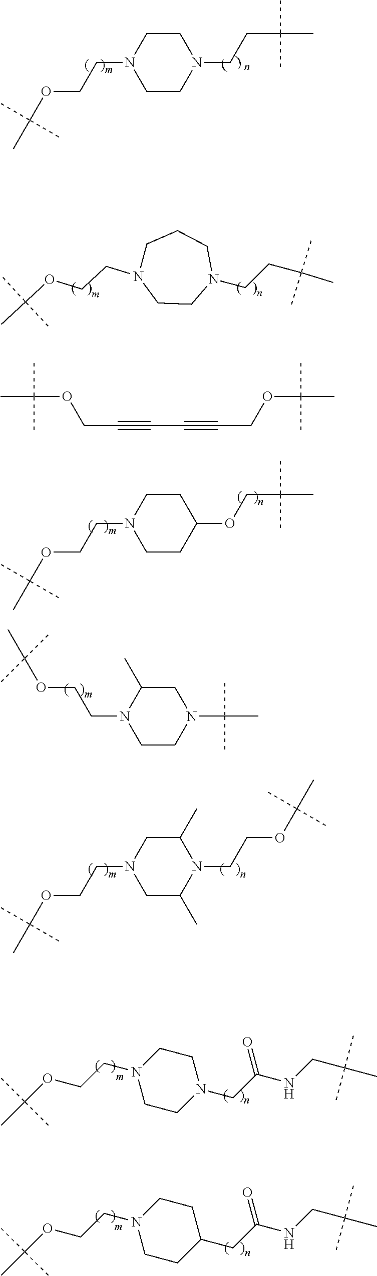

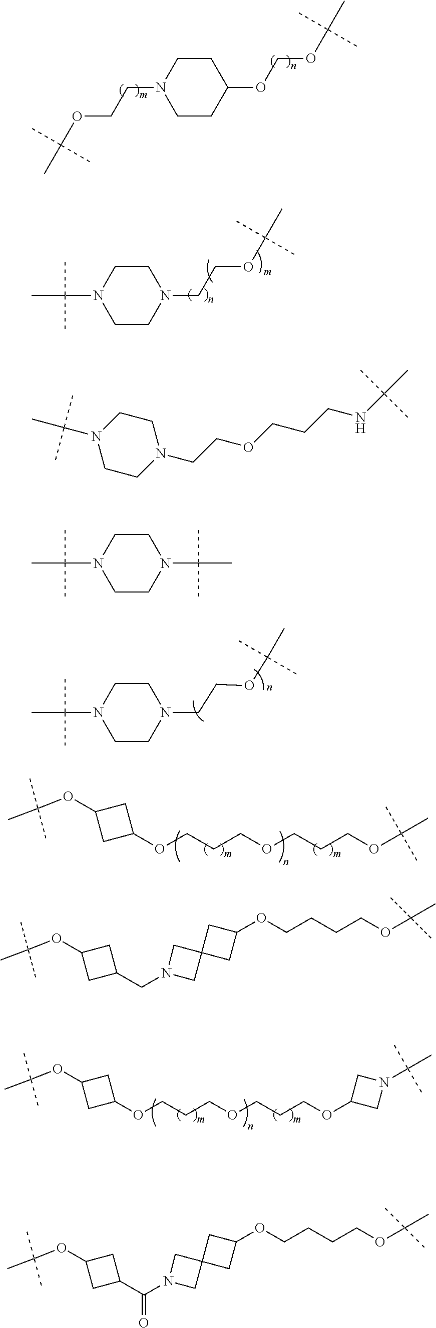

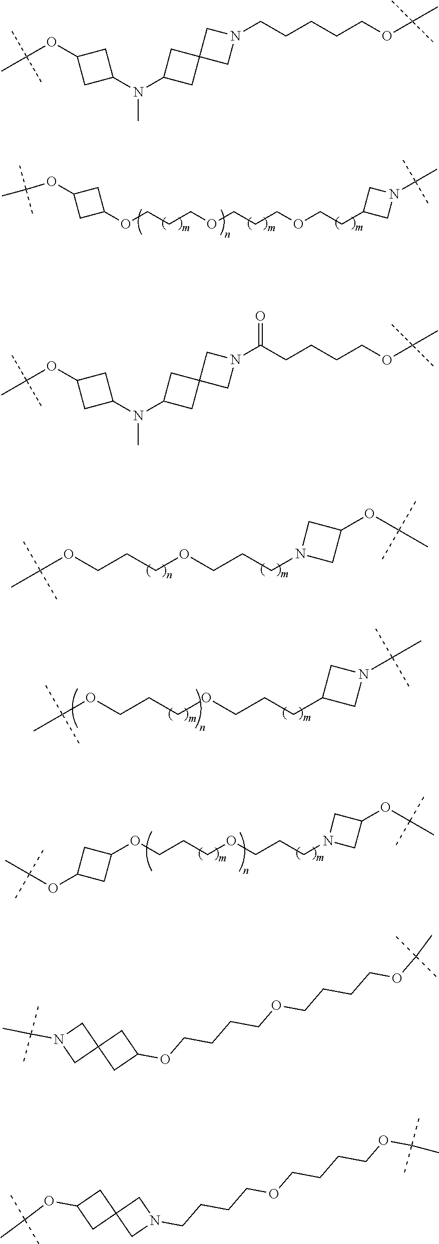

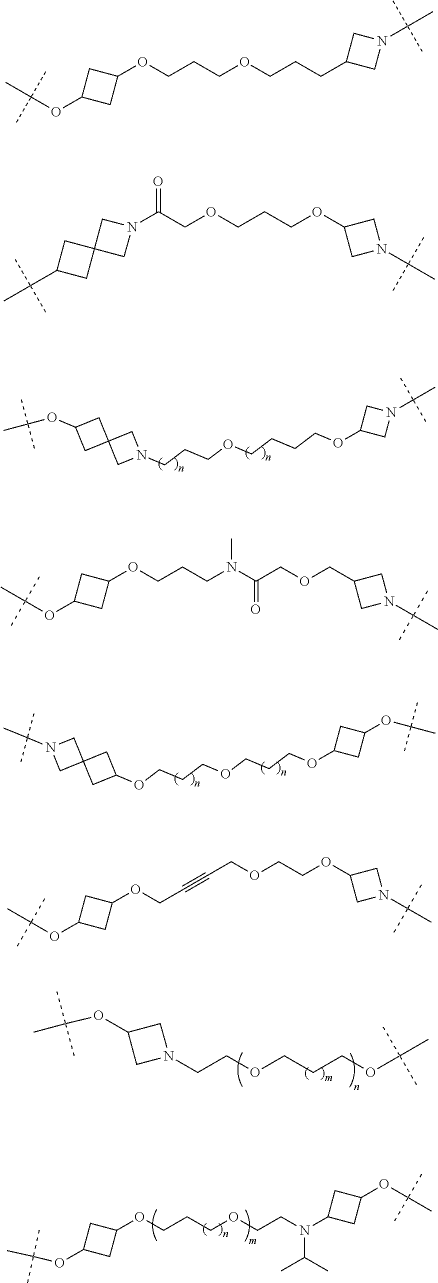

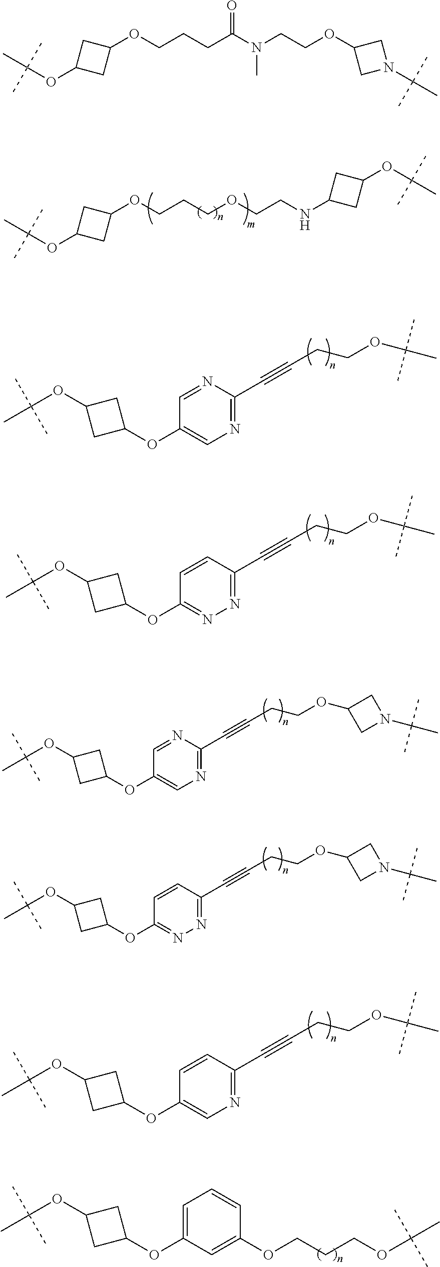

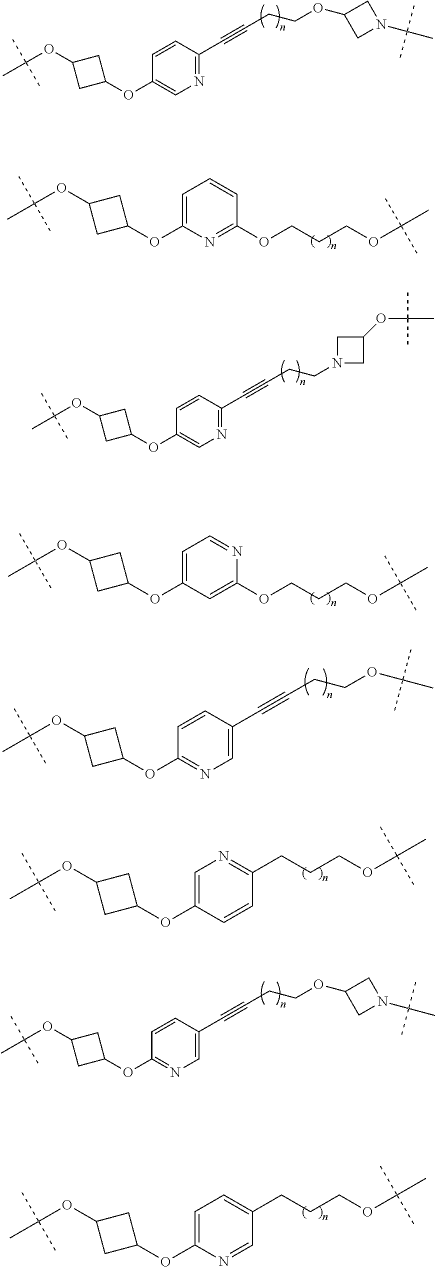

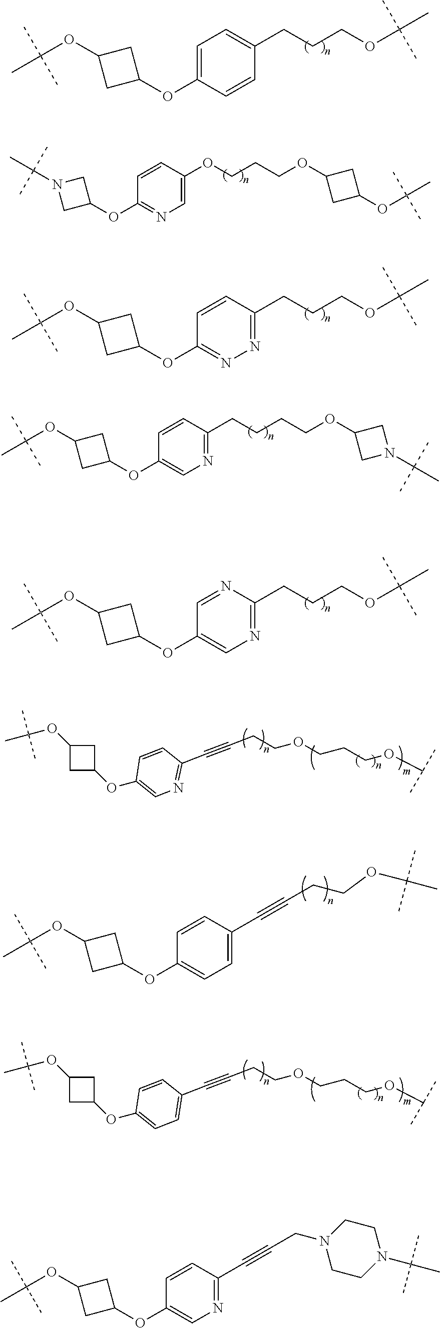

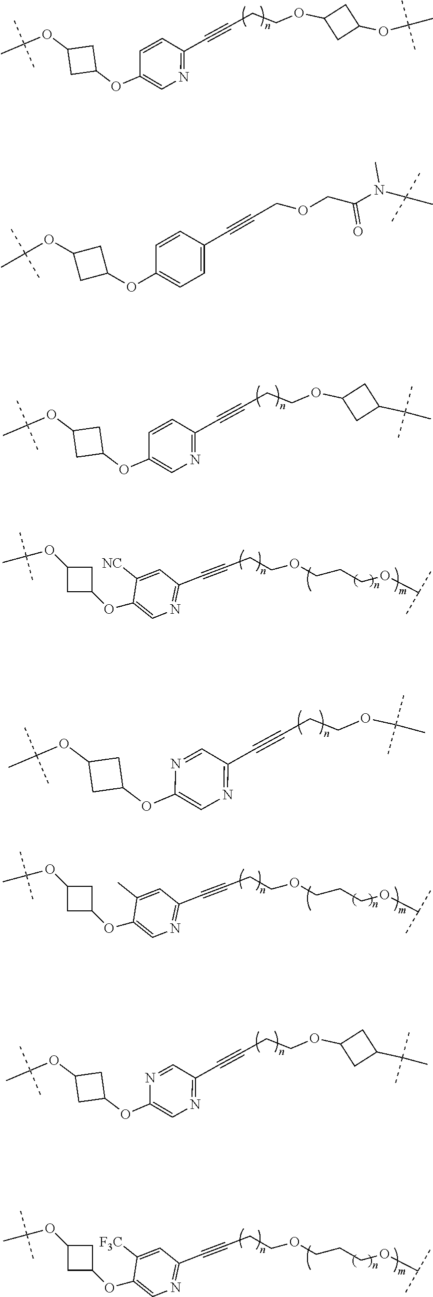

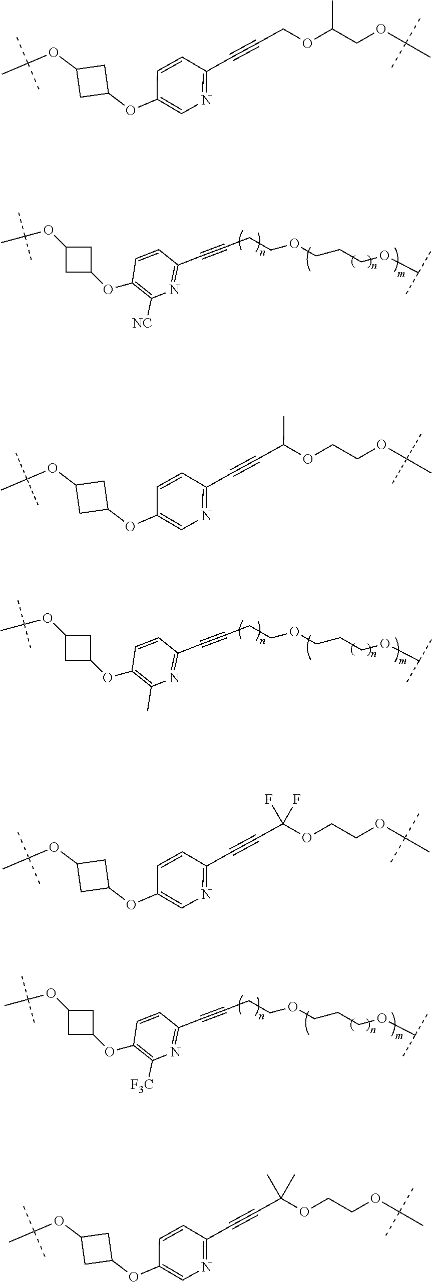

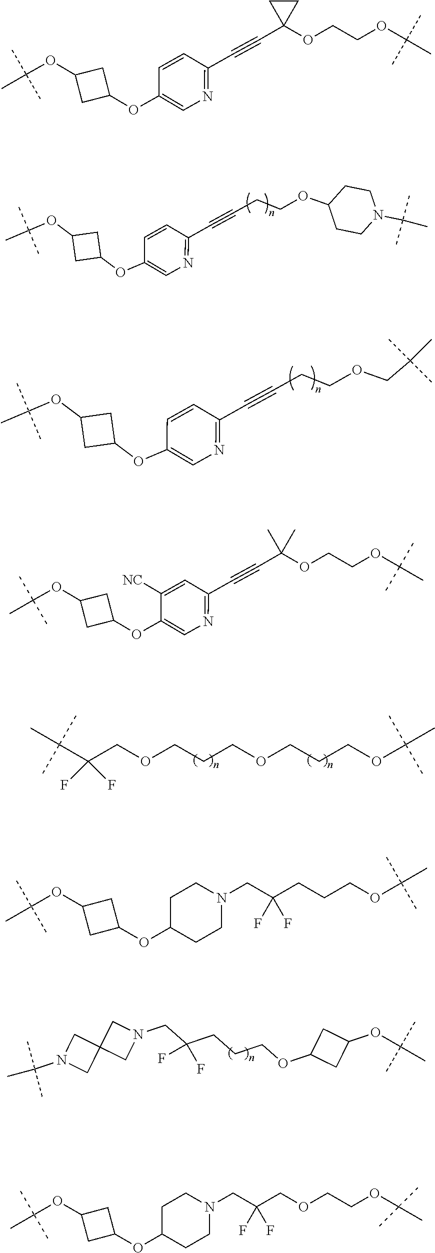

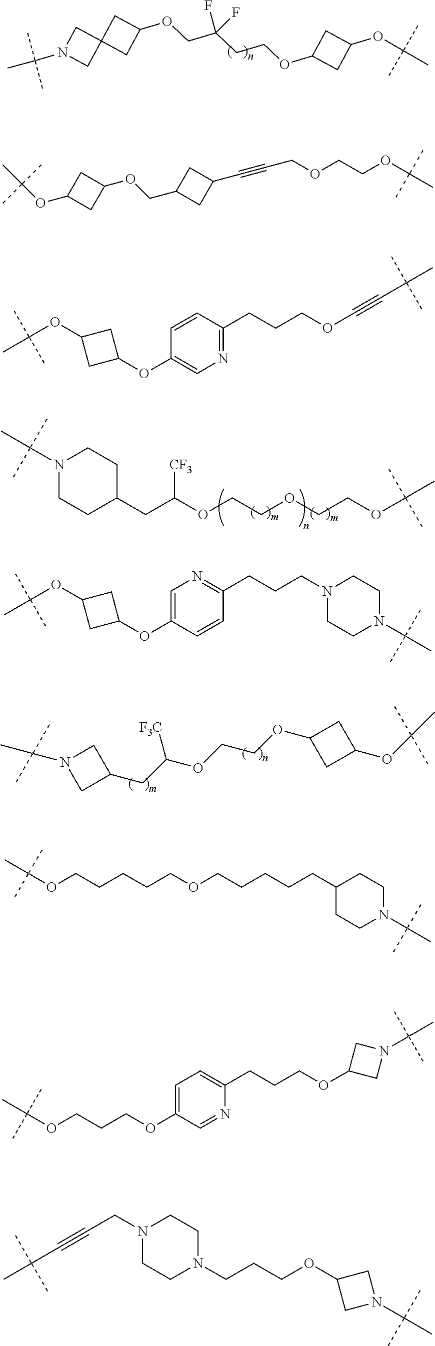

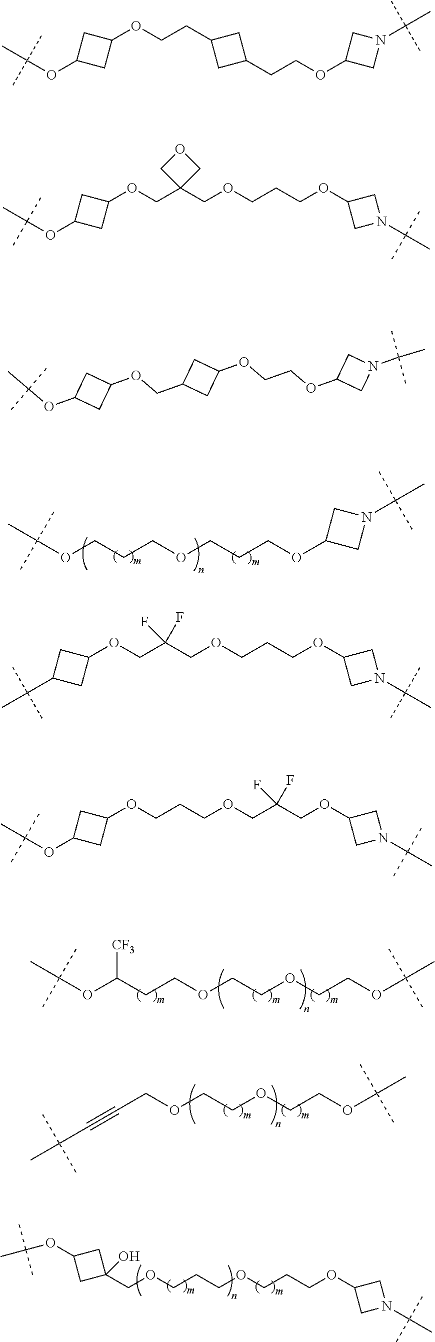

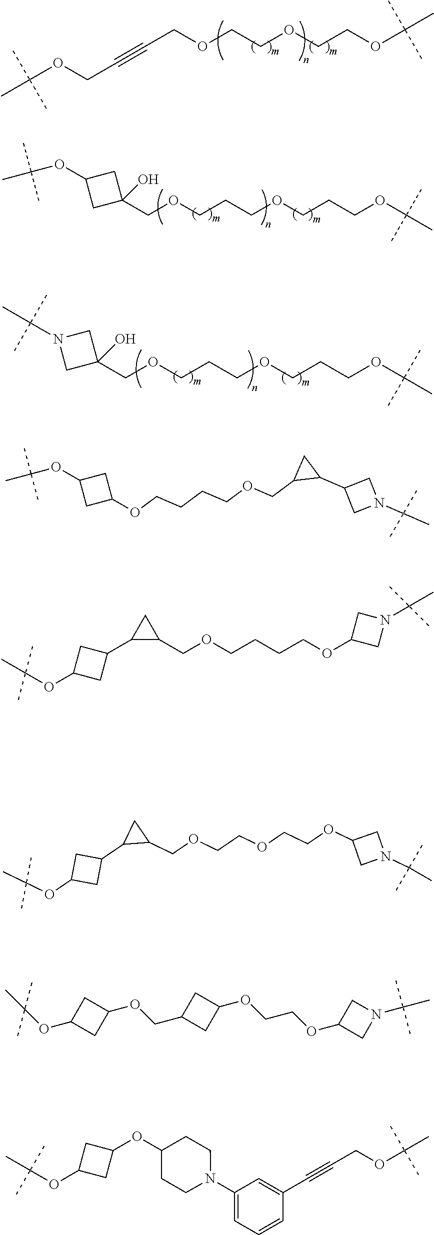

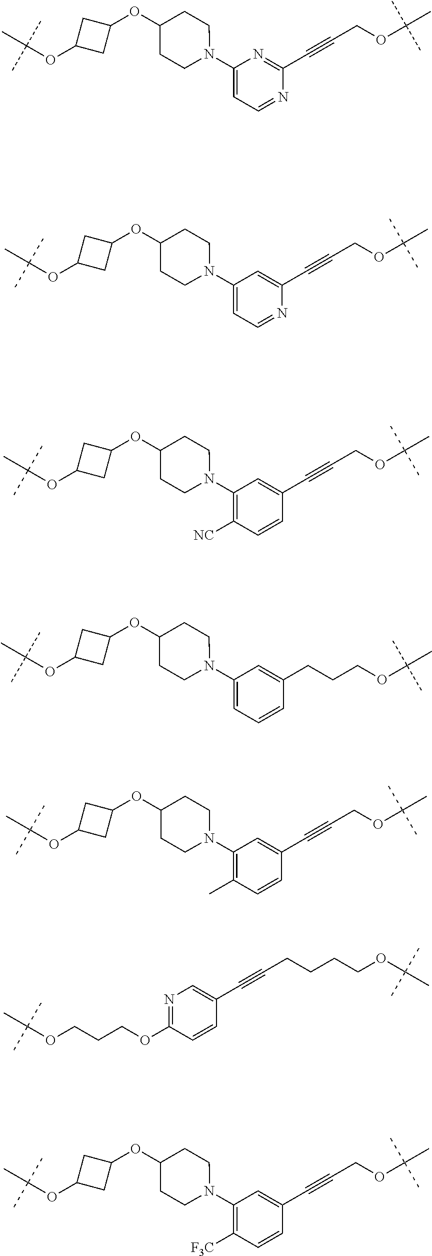

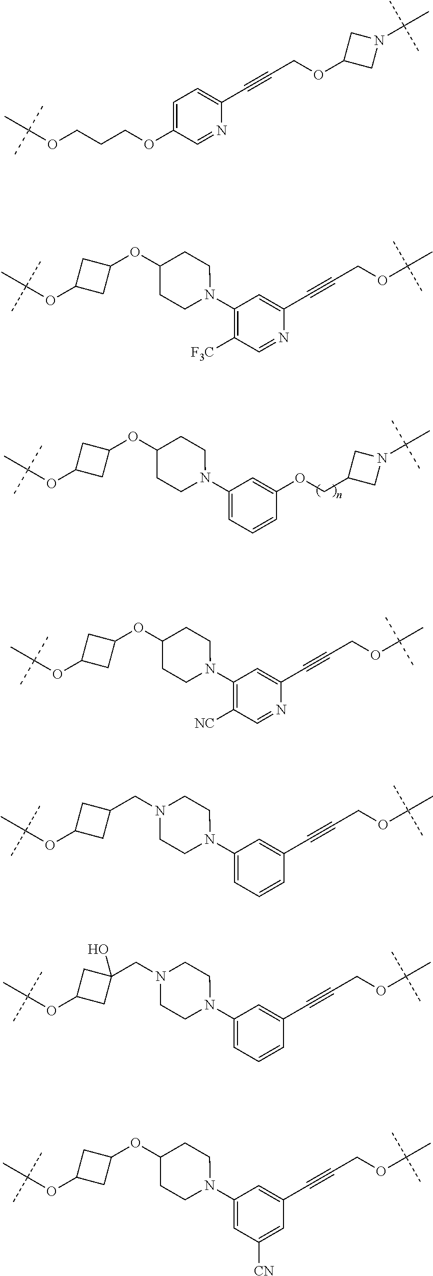

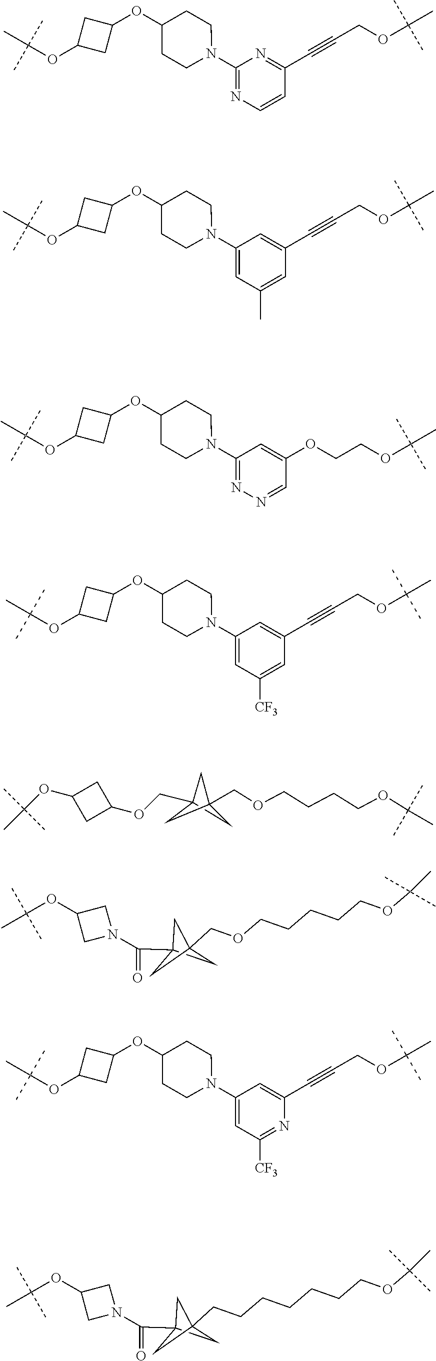

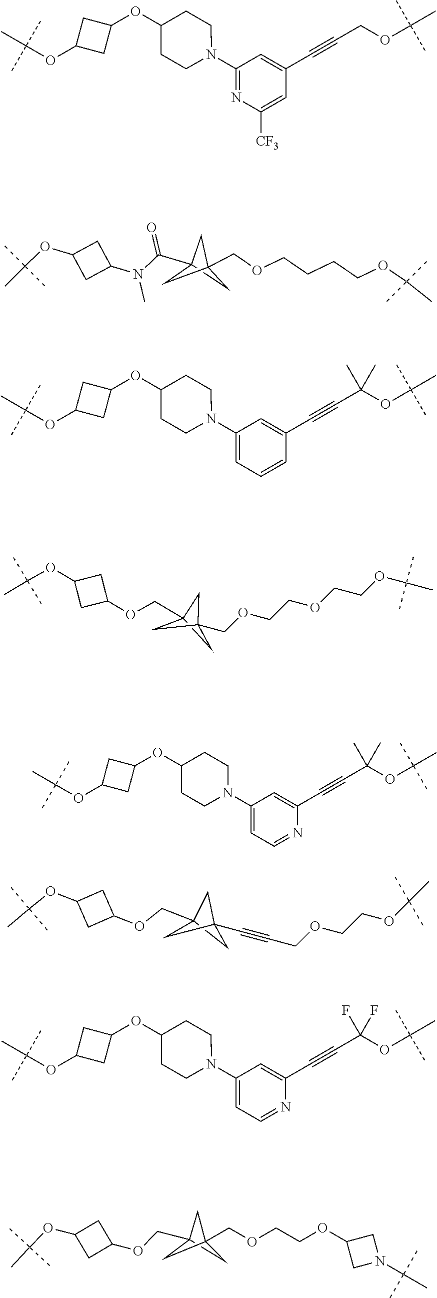

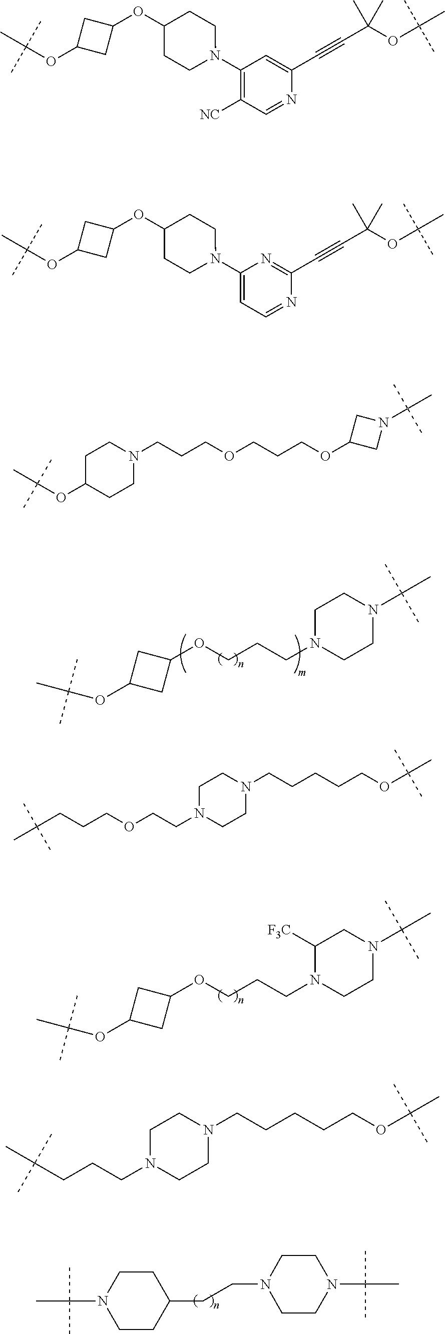

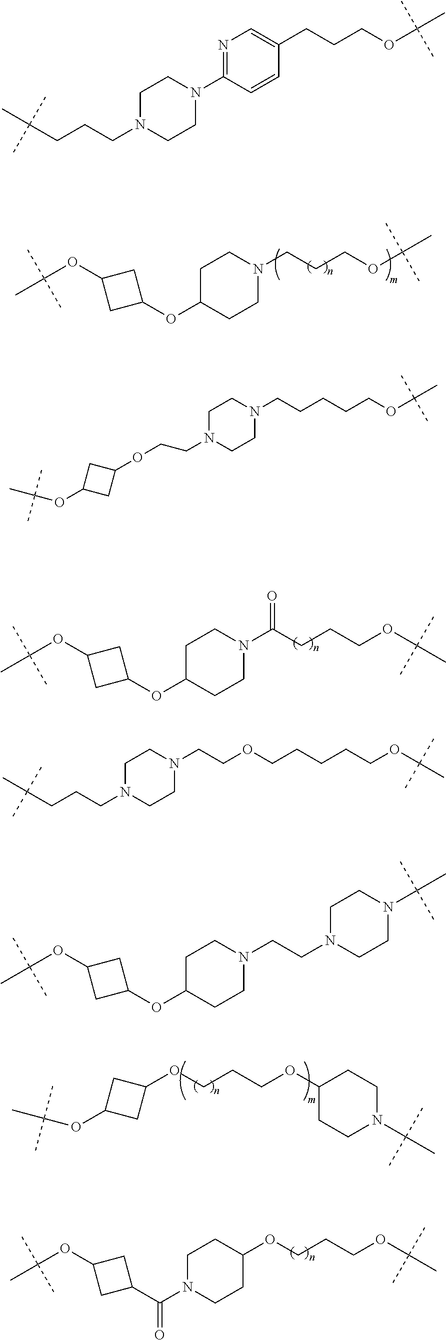

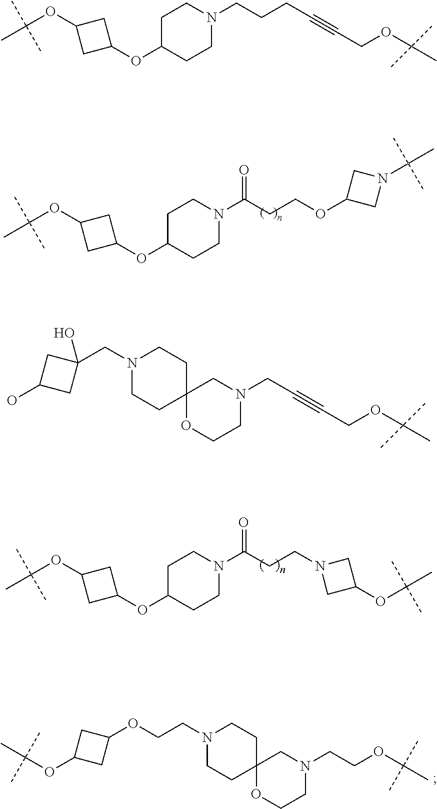

wherein each instance of m, n, o, and p in the linker is independently 0, 1, 2, 3, 4, 5, 6, 7, 8, 9, 10, 11, 12, 13, 14, 15, 16, 17, 18, 19, or 20; and a dashed bond indicates a point of attachment to the protein phosphatase ligand or the target protein ligand.

In some embodiments, the linker is one of the following:

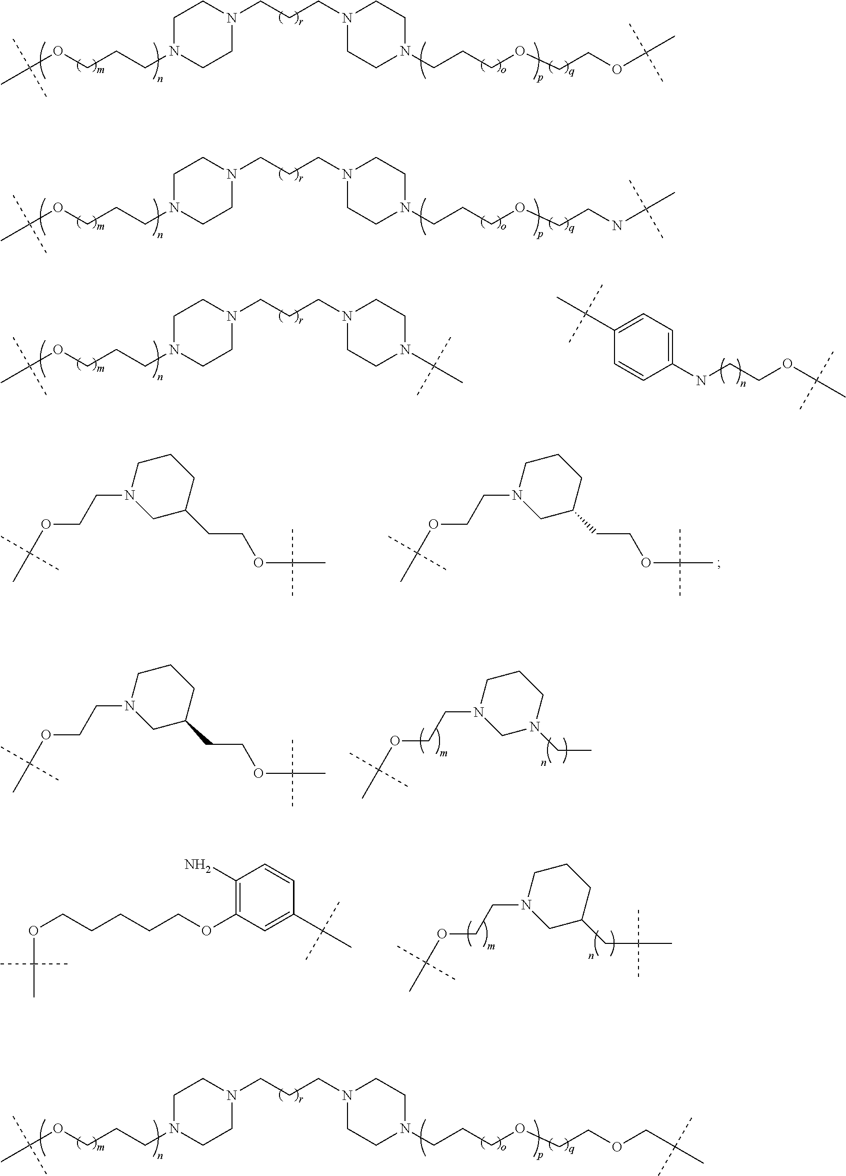

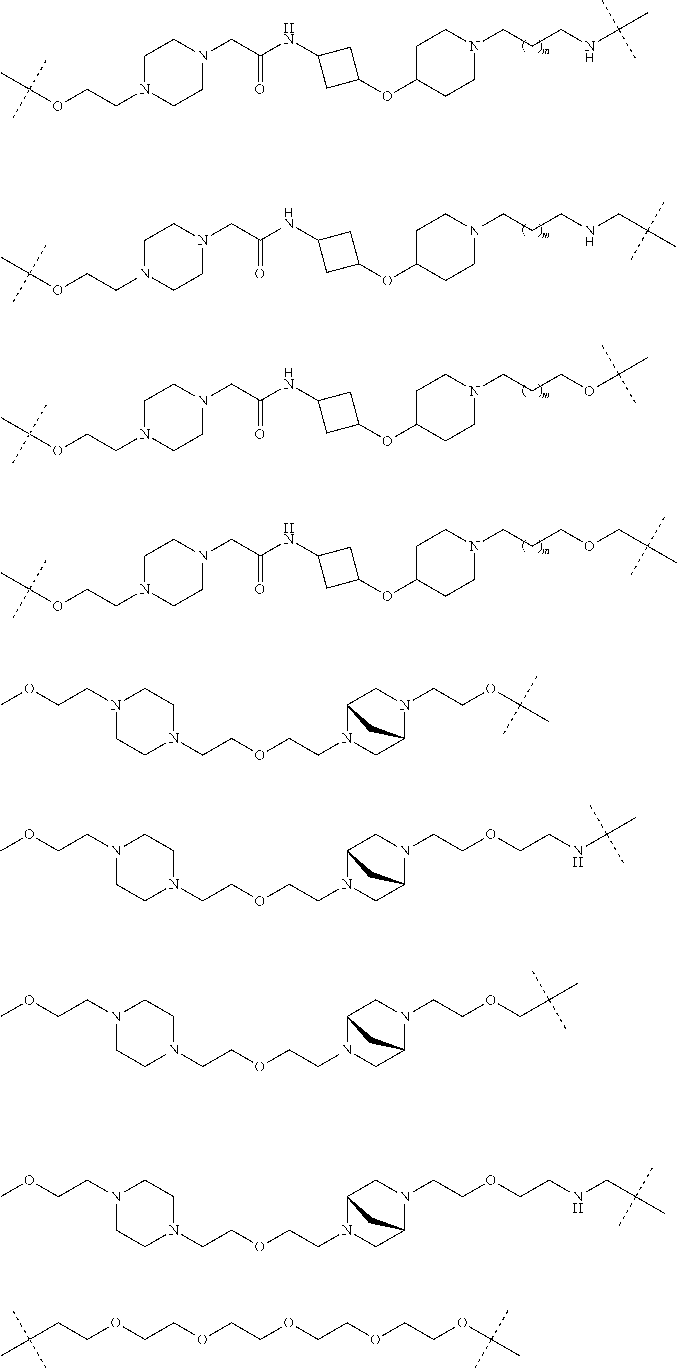

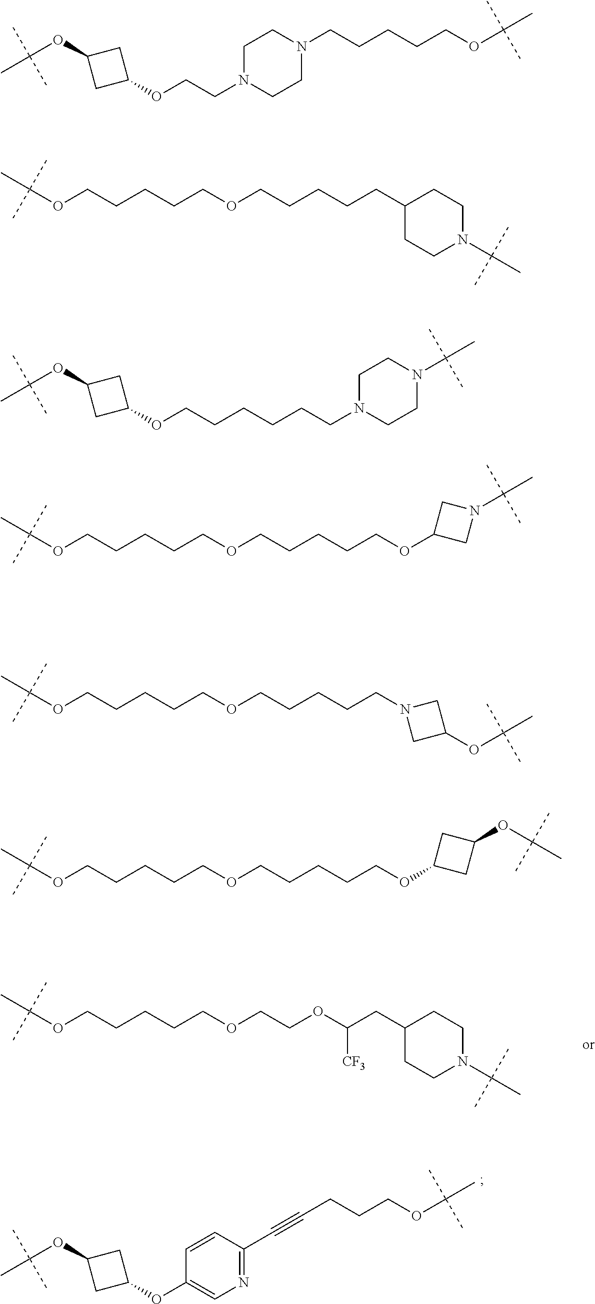

wherein each m, n, o, p, q, and r in the linker is independently 0, 1, 2, 3, 4, 5, 6, 7, 8, 9, 10, 11, 12, 13, 14, 15, 16, 17, 18, 19, or 20; and a dashed bond indicates a point of attachment to the protein phosphatase ligand or the target protein ligand.

In some embodiments, the linker is one of the following:

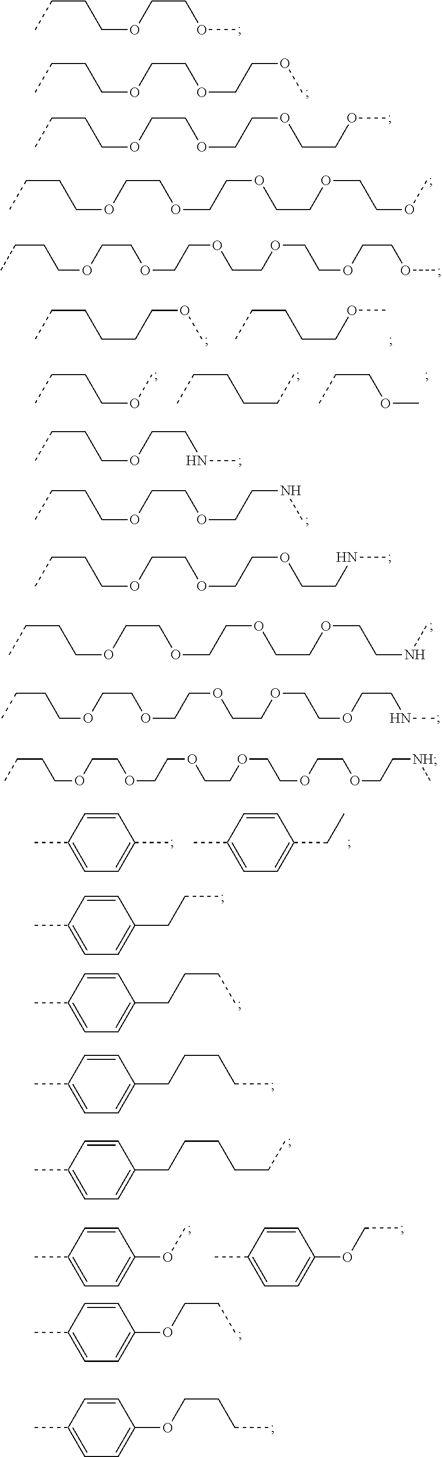

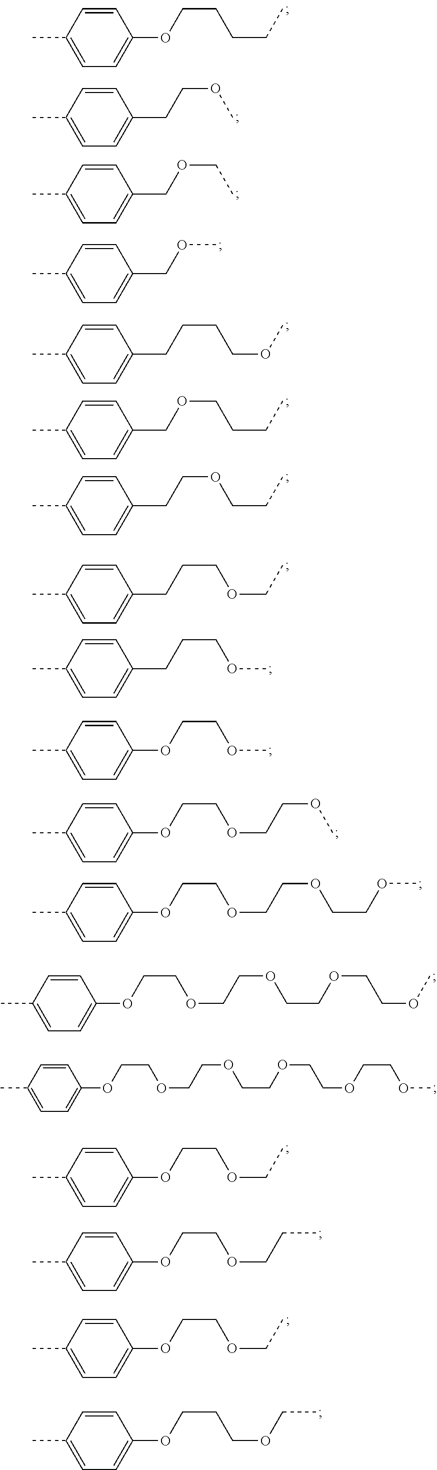

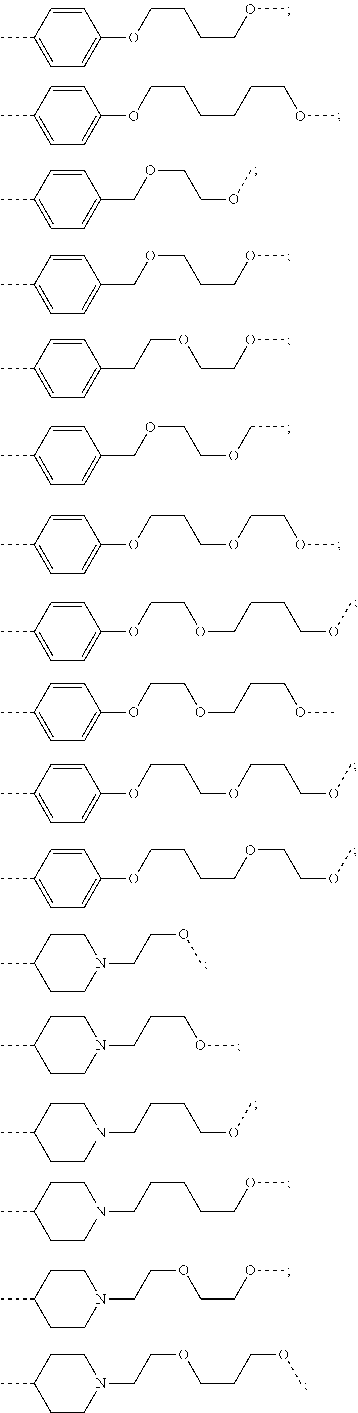

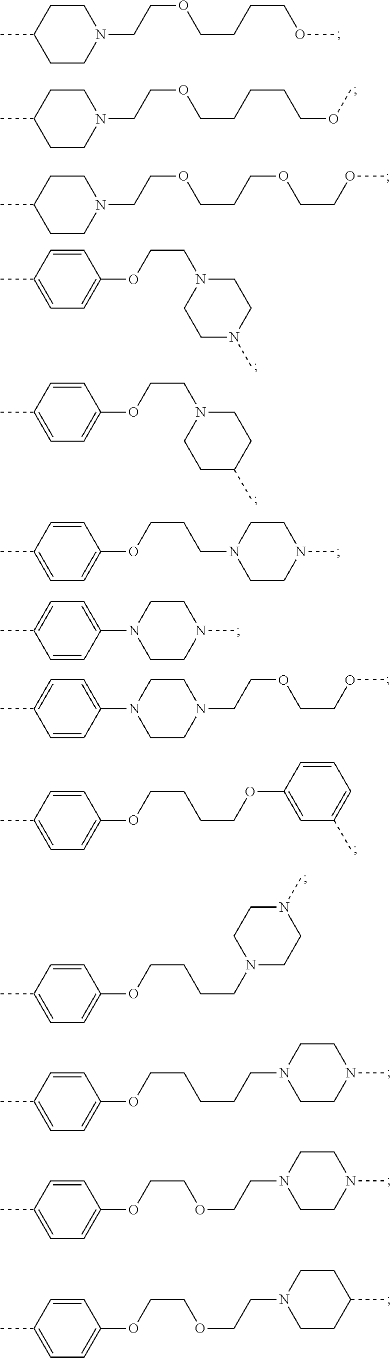

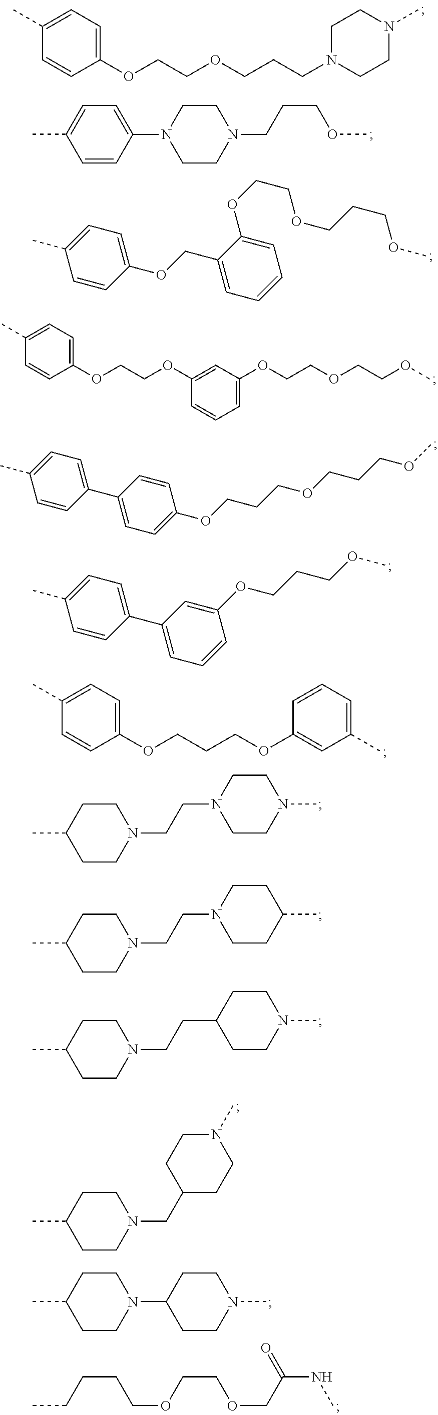

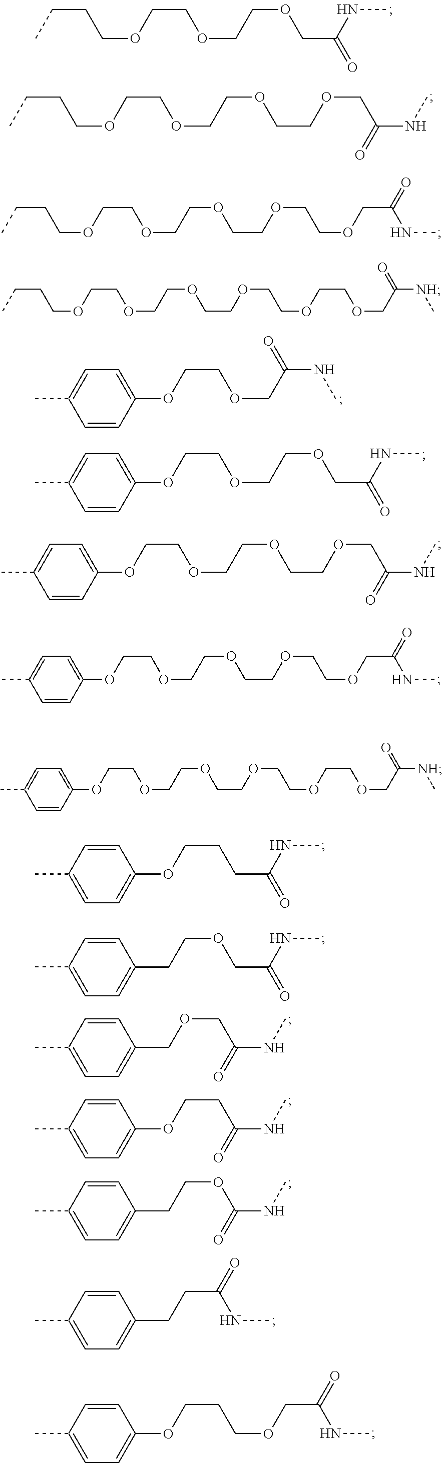

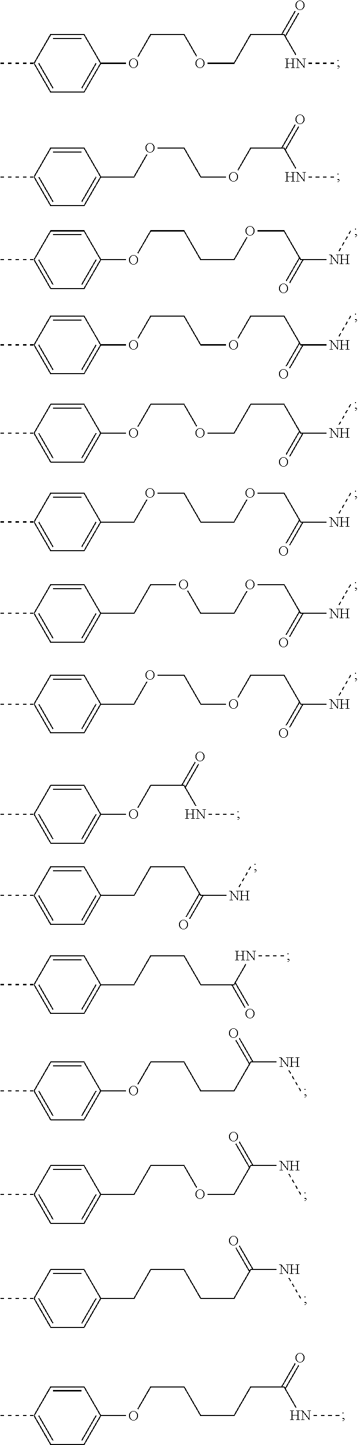

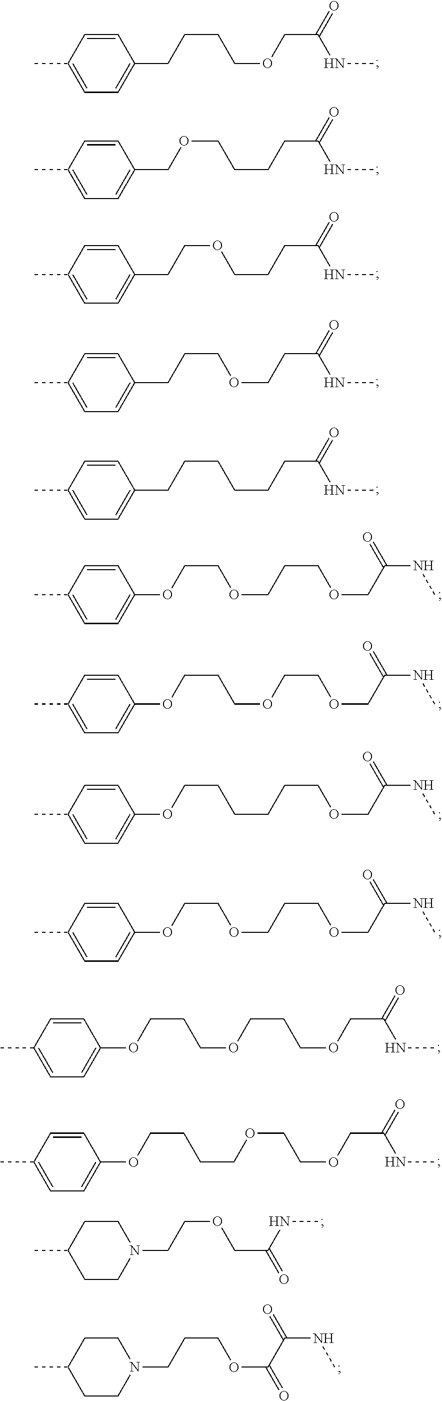

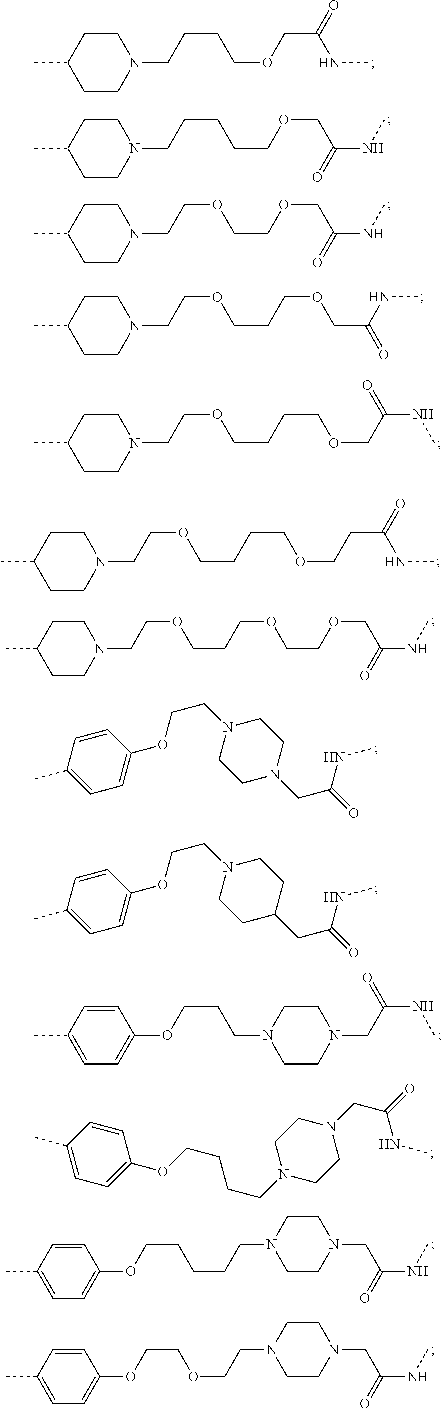

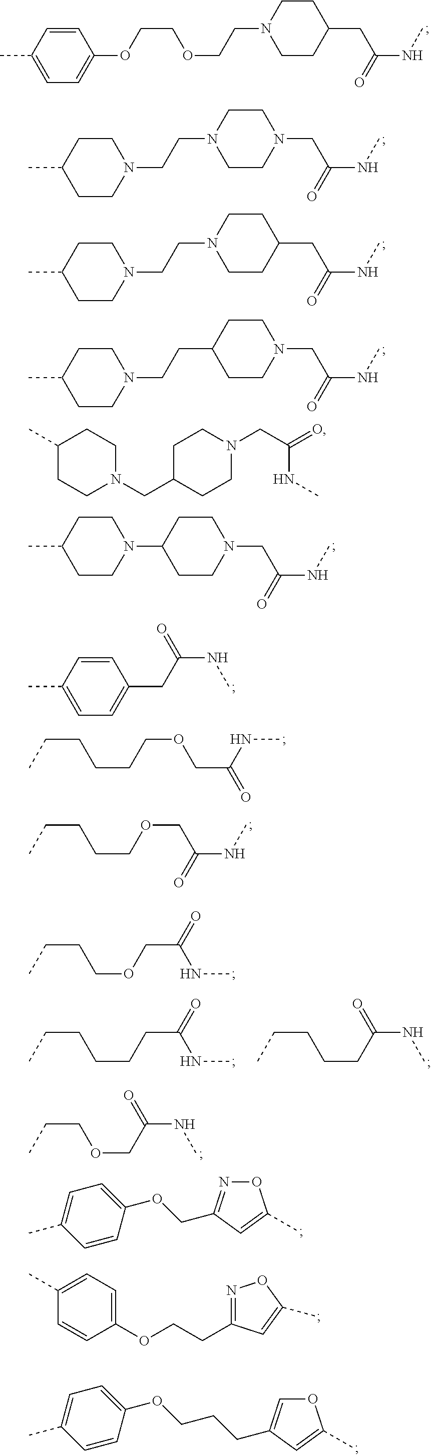

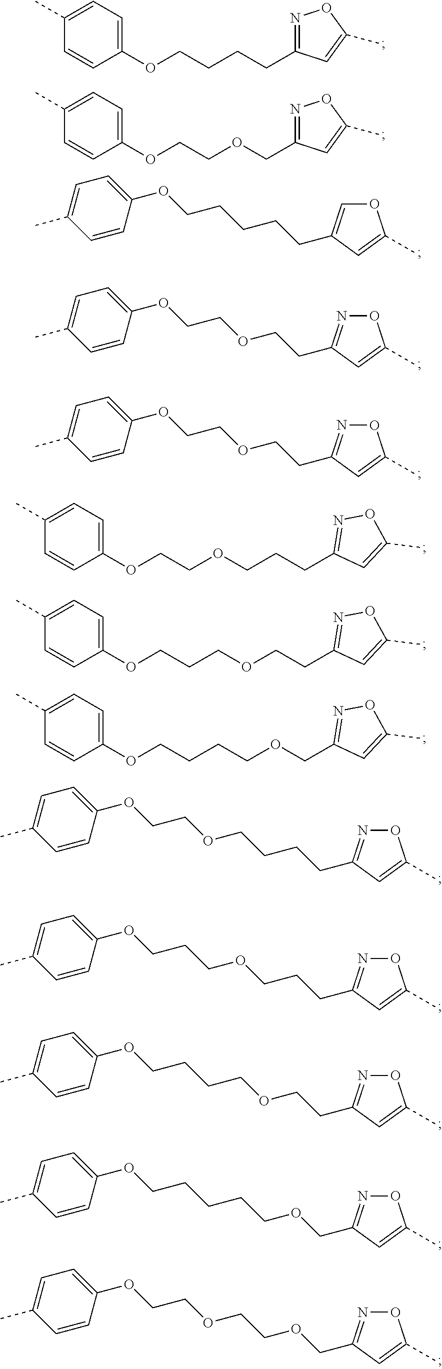

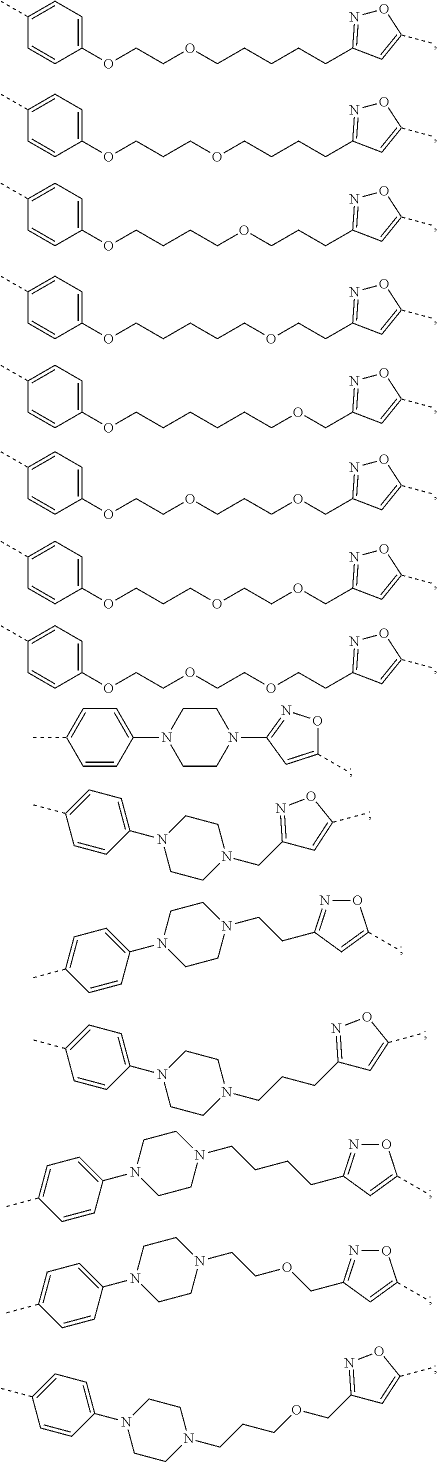

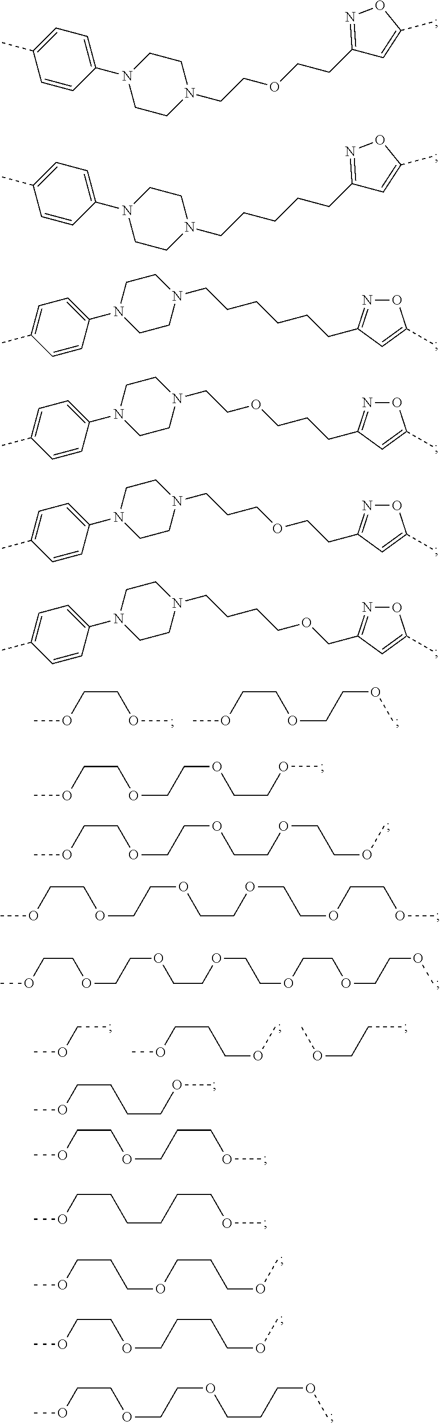

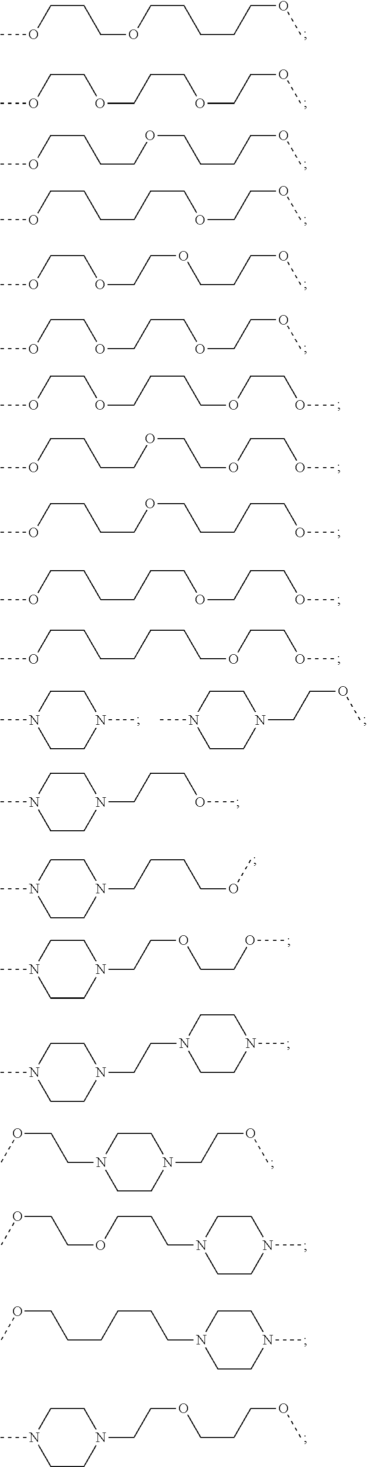

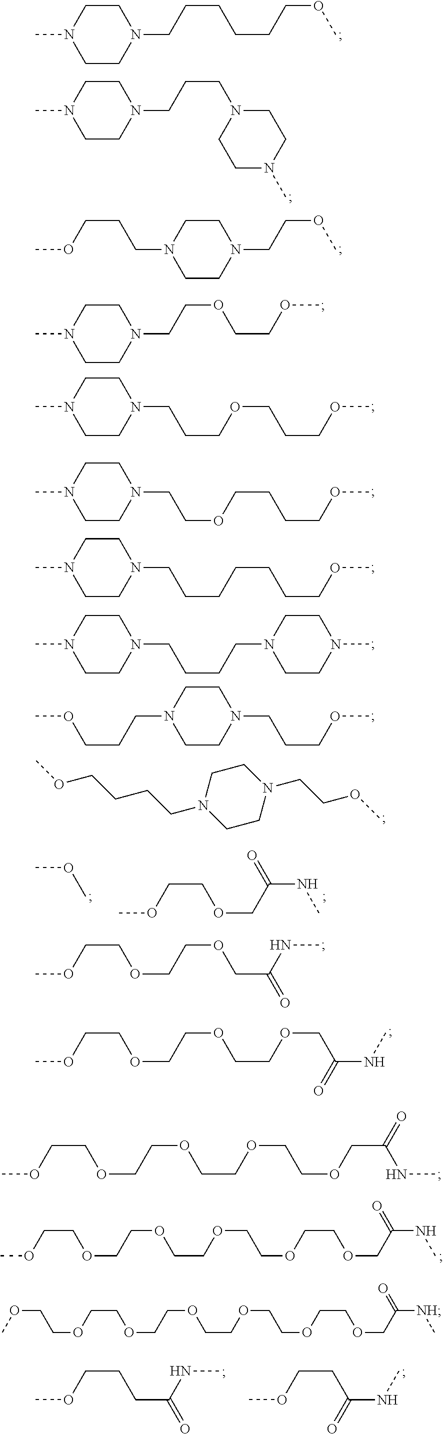

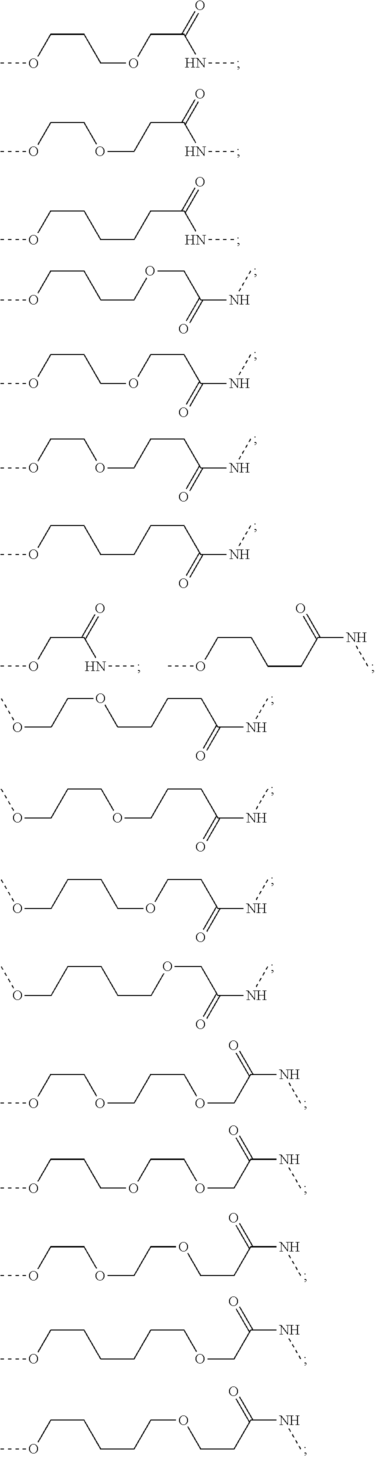

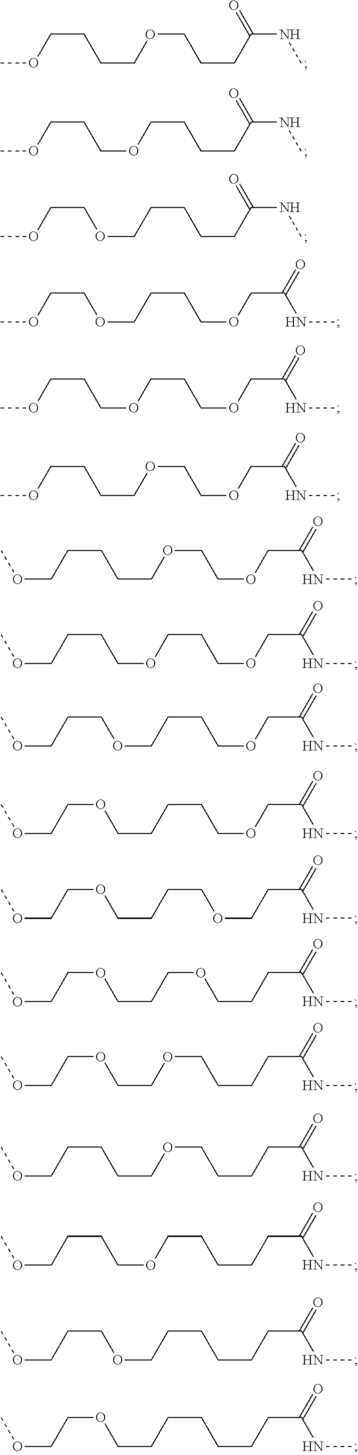

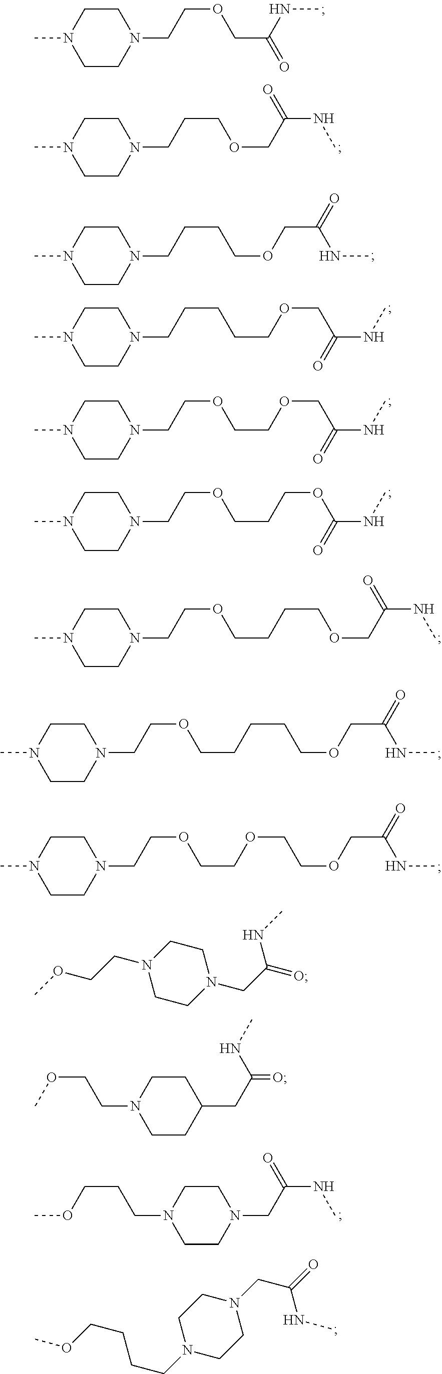

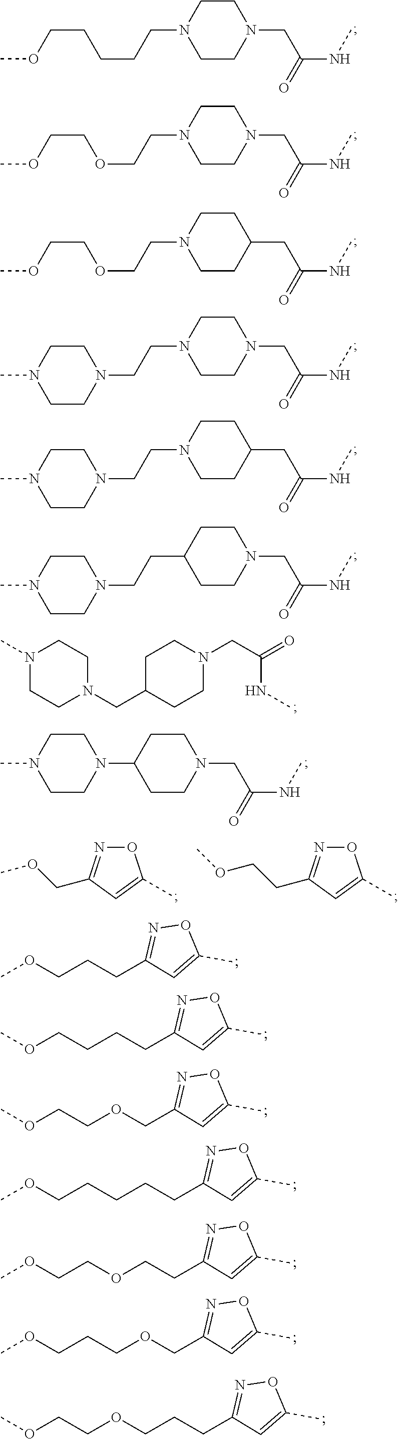

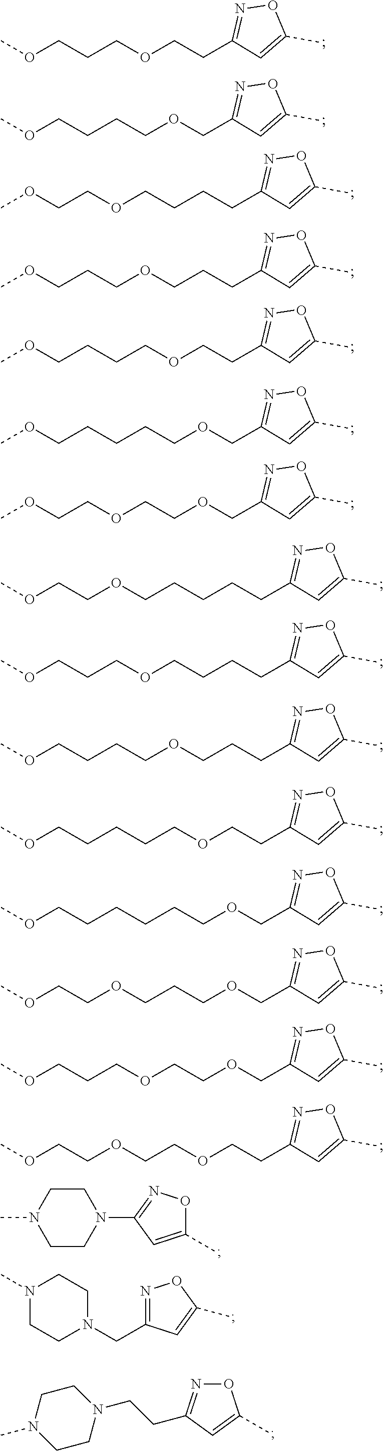

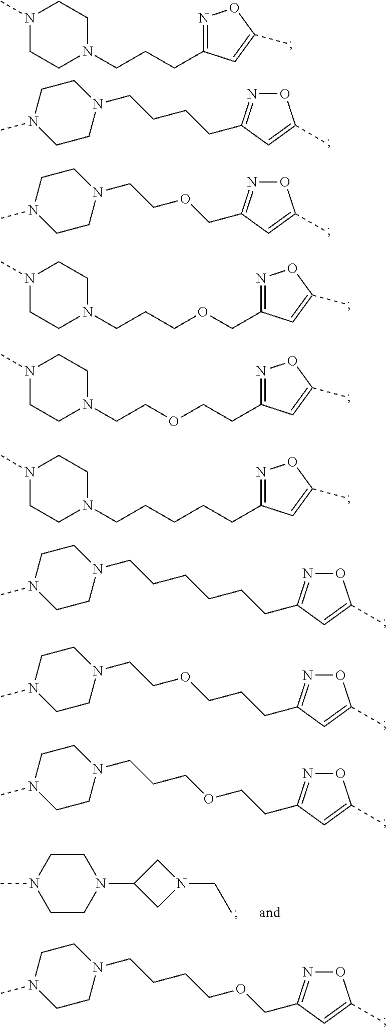

wherein a dashed bond indicates a point of attachment to the protein phosphatase ligand or the target protein ligand.

In some embodiments, the linker is one of the following:

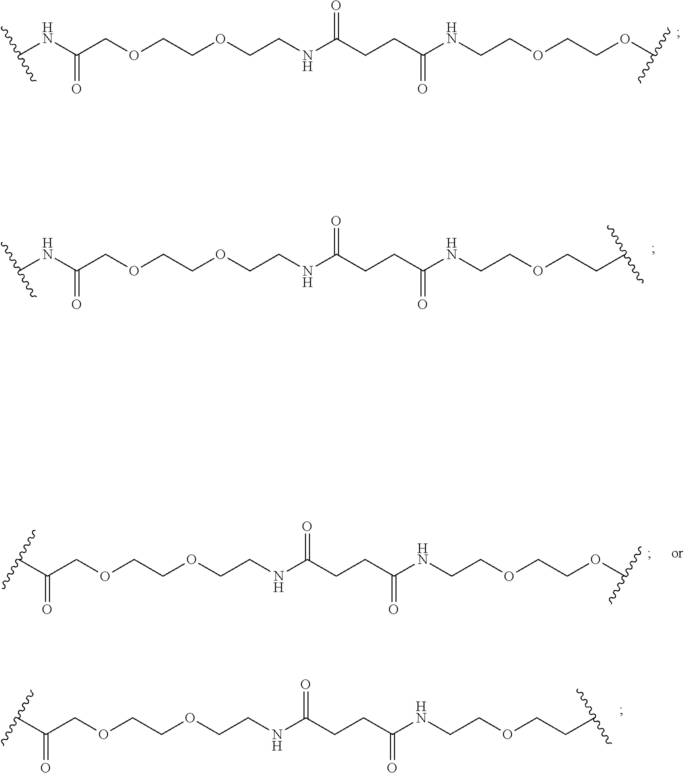 [SMILES: *NC(=O)COCCOCCNC(=O)CCC(=O)NCCOCCO* ; *CCOCCNC(=O)CCC(=O)NCCOCCOCC(=O)N* ; *OCCOCCNC(=O)CCC(=O)NCCOCCOCC(*)=O ; *CCOCCNC(=O)CCC(=O)NCCOCCOCC(*)=O]

wherein the symbol “ [symbol] v” indicates a point of attachment to the protein phosphatase ligand or the target protein ligand.

In some embodiments, the linker is selected from the group consisting of:

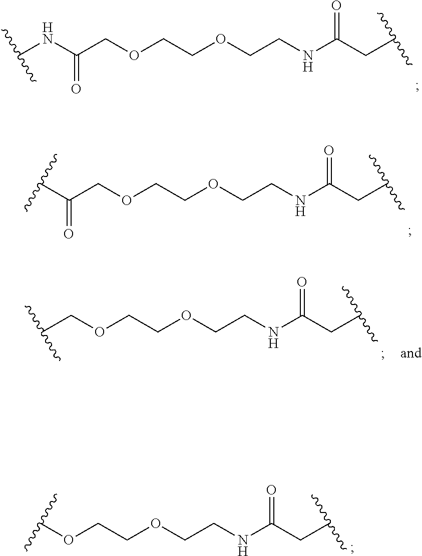 [SMILES: *CC(=O)NCCOCCOCC(=O)N* ; *CC(=O)NCCOCCOCC(*)=O ; *COCCOCCNC(=O)C* ; *CC(=O)NCCOCCO*]

wherein the symbol “ [symbol] ” indicates a point of attachment to the protein phosphatase ligand or the target protein ligand.

In some embodiments, the linker is selected from the group consisting of:

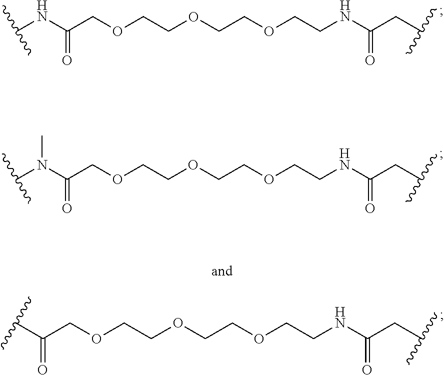 [SMILES: *CC(=O)NCCOCCOCCOCC(=O)N* ; *CC(=O)NCCOCCOCCOCC(=O)N(*)C ; *CC(=O)NCCOCCOCCOCC(*)=O]

wherein the symbol “ [symbol] ” indicates a point of attachment to the protein phosphatase ligand or the target protein ligand.

In some embodiments, the linker is selected from the group consisting of:

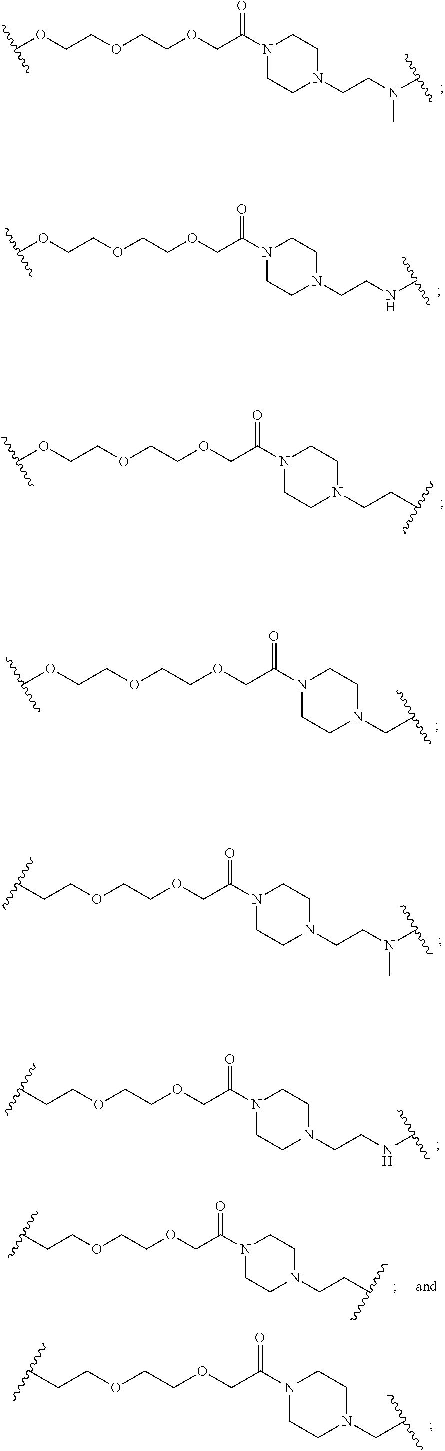

wherein the symbol “ [symbol] ” indicates a point of attachment to the protein phosphatase ligand or the target protein ligand.

The linker of the present invention can be covalently bonded to the target protein ligand and protein phosphatase ligand through any group which is appropriate and stable to the chemistry of the linker. In some embodiments, the linker of the present invention can be covalently bonded to the target protein ligand and protein phosphatase ligand, suitably through an amide, ester, thioester, keto group, carbamate (urethane), carbon, or ether. In some embodiments, the linker of the present invention can be covalently bonded to the target protein ligand and protein phosphatase ligand, suitable through an amide, ester, thioester, keto group, carbamate (urethane) or ether. The linking position can be anywhere in the target protein ligand and protein phosphatase ligand. One of ordinary skill in the art would recognize the suitable linking positions to maximize the binding affinity between the target protein ligand and the target protein, and between the protein phosphatase ligand and the protein phosphatase. In certain embodiments, the linker may be linked to an optionally substituted alkyl, alkylene, alkene or alkyne group, an aryl group or a heterocyclic group on the target protein ligand and/or protein phosphatase ligand.

In certain embodiments, the linker is the linker component in one of the compounds set forth in Tables 1, 3, 4, 6, 7, 9, 11, 12, 14-16, or 18-20. In certain embodiments, the linker is the linker component in one of the compounds set forth in Tables 1, 3, 4, 6, 7, 9, 11, 12, 14-16, 18-20, or 22. In certain embodiments, the linker is the linker component in one of the compounds set forth in Tables 1, 3, 4, 6, 7, 9, 11, 12, 14A, 16A, or 22.

Exemplary More Specific Embodiment of Compounds

In certain embodiments, the compound is represented by one of the following formulae, or a pharmaceutically acceptable salt thereof:

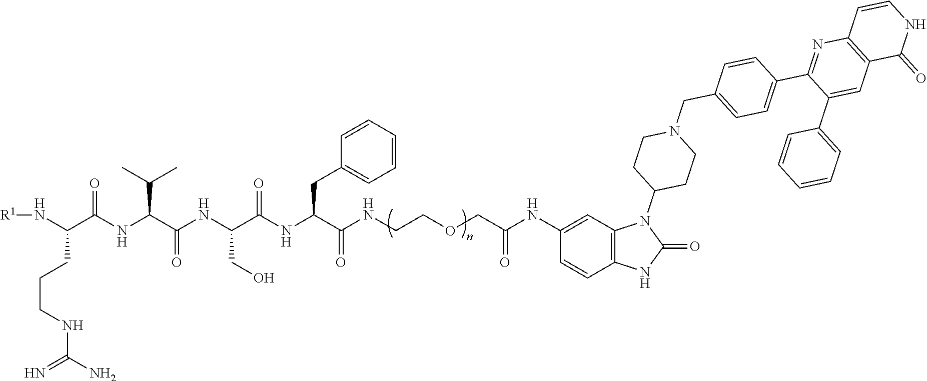

wherein R¹ is hydrogen or —C(O)CH₃, and n is 0, 1, 2, 3, or 4;

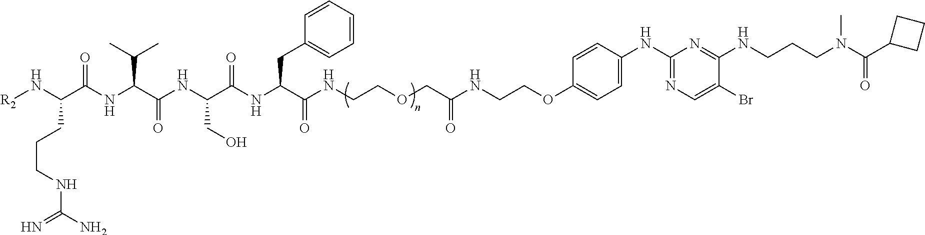

wherein R² is hydrogen, —C(O)CH₃, or —C(O)(CH₂)CH₃ and n is 0, 1, 2, 3, or 4 or

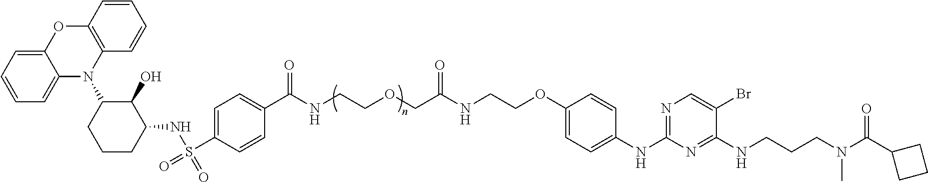

wherein n is 0, 1, 2, 3, or 4.

Preparation and General Features of Compounds

Compounds of the invention can be prepared by the general schemes and/or procedures described herein, using the synthetic method known by those skilled in the art.

The compounds of the invention may possess one or more stereocenters, and each stereocenter may exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound of the invention, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compounds of the invention may exist as tautomers. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compounds of the invention are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000, 2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from:

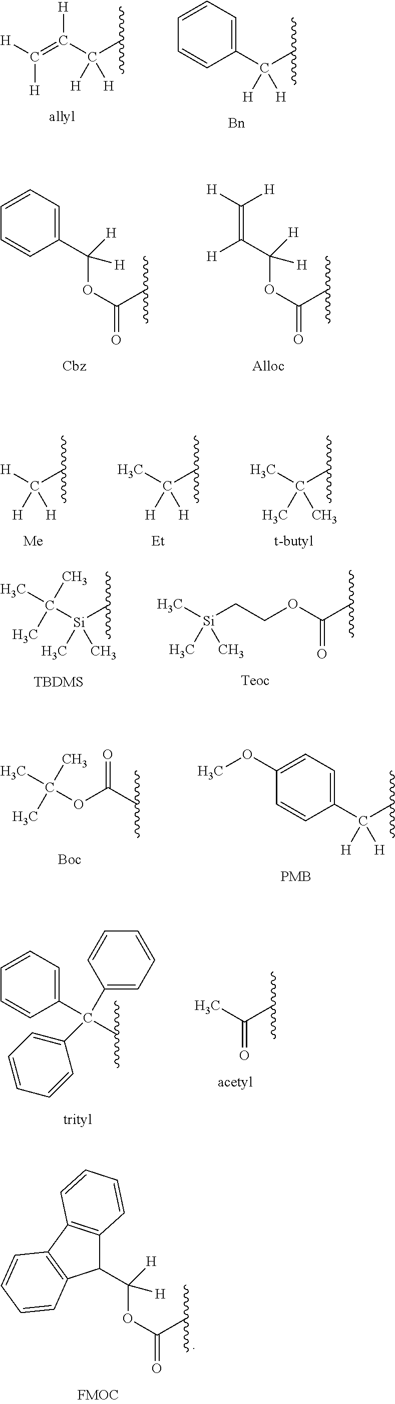

Other protecting groups, plus a detailed description of techniques applicable to the creation of protecting groups and their removal are described in Greene & Wuts, Protective Groups in Organic Synthesis, 3rd Ed., John Wiley & Sons, New York, N.Y., 1999, and Kocienski, Protective Groups, Thieme Verlag, New York, N.Y., 1994, which are incorporated herein by reference for such disclosure.

### Compositions

The invention includes a pharmaceutical composition comprising at least one compound of the invention and at least one pharmaceutically acceptable carrier. In certain embodiments, the composition is formulated for an administration route such as oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

### Methods

The invention includes a method of treating or preventing a disease associated with and/or caused by overphosphorylation, undesirable phosphorylation, and/or uncontrolled phosphorylation of a target protein in a subject. The invention further includes a method of treating or preventing a cancer associated with and/or caused by overphosphorylation, undesirable phosphorylation, and/or uncontrolled phosphorylation of a target protein in a subject. In certain embodiments, the disease comprises cancer, neurodegeneration, metabolic disease, diabetes, and/or insulin resistance.

Accordingly, one aspect of the invention provides a method of treating or preventing a disease associated with and/or caused by overphosphorylation, undesirable phosphorylation, and/or uncontrolled phosphorylation of a target protein in a subject, wherein the method comprises administering to the subject a therapeutically effective amount of at least one compound described herein. In certain embodiments, the disease or disorder comprises cancer, neurodegeneration, metabolic disease, diabetes, and/or insulin resistance. In certain embodiments, the disease or disorder is cancer.

Another aspect of the invention provides a method of treating or preventing a disease associated with and/or caused by overphosphorylation, undesirable phosphorylation, and/or uncontrolled phosphorylation of a target protein in a subject, wherein the method comprises administering to the subject in need thereof a therapeutically effective amount of at least one compound described herein. In certain embodiments, the disease or disorder comprises cancer, neurodegeneration, metabolic disease, diabetes, and/or insulin resistance. In certain embodiments, the disease or disorder is cancer.

Examples of cancers that can be treated or prevented by the present invention include but are not limited to: squamous cell cancer, lung cancer including small cell lung cancer, non-small cell lung cancer, vulval cancer, thyroid cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, hepatic carcinoma, anal carcinoma, penile carcinoma, and head and neck cancer. In certain embodiments, the cancer is at least one selected from the group consisting of ALL, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL, Philadelphia chromosome positive CML, lymphoma, leukemia, multiple myeloma myeloproliferative diseases, large B cell lymphoma, and B cell Lymphoma.

In certain embodiments, the cancer is a solid tumor or leukemia. In certain other embodiments, the cancer is colon cancer, pancreatic cancer, breast cancer, ovarian cancer, prostate cancer, squamous cell carcinoma, basal cell carcinoma, adenocarcinoma, sweat gland carcinoma, sebaceous gland carcinoma, lung cancer, leukemia, bladder cancer, stomach cancer, cervical cancer, testicular cancer, skin cancer, rectal cancer, thyroid cancer, kidney cancer, uterus cancer, esophagus cancer, liver cancer, an acoustic neuroma, oligodendroglioma, meningioma, neuroblastoma, or retinoblastoma. In certain other embodiments, the cancer is small cell lung cancer, non-small cell lung cancer, melanoma, cancer of the central nervous system tissue, brain cancer, Hodgkin's lymphoma, non-Hodgkin's lymphoma, cutaneous T-Cell lymphoma, cutaneous B-Cell lymphoma, or diffuse large B-Cell lymphoma. In certain other embodiments, the cancer is breast cancer, colon cancer, small-cell lung cancer, non-small cell lung cancer, prostate cancer, renal cancer, ovarian cancer, leukemia, melanoma, or cancer of the central nervous system tissue. In certain other embodiments, the cancer is colon cancer, small-cell lung cancer, non-small cell lung cancer, renal cancer, ovarian cancer, renal cancer, or melanoma.

In certain embodiments, the cancer is a fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, epithelial carcinoma, glioma, astrocytoma, medulloblastoma, and hemangioblastoma.

In certain embodiments, the cancer is a neuroblastoma, meningioma, hemangiopericytoma, multiple brain metastase, glioblastoma multiforms, glioblastoma, brain stem glioma, poor prognosis malignant brain tumor, malignant glioma, anaplastic astrocytoma, anaplastic oligodendroglioma, neuroendocrine tumor, rectal adeno carcinoma, Dukes C & D colorectal cancer, unresectable colorectal carcinoma, metastatic hepatocellular carcinoma, Kaposi's sarcoma, karotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, metastatic melanoma, localized melanoma, malignant mesothelioma, malignant pleural effusion mesothelioma syndrome, peritoneal carcinoma, papillary serous carcinoma, gynecologic sarcoma, soft tissue sarcoma, scelroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, Waidenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, or leiomyoma.

In certain embodiments, the cancer is bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, non-Hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In certain embodiments, the cancer is selected from hepatocellular carcinoma, ovarian cancer, ovarian epithelial cancer, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical adenoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In certain embodiments, the cancer is selected from hepatocellular carcinoma (HCC), hepatoblastoma, colon cancer, rectal cancer, ovarian cancer, ovarian epithelial cancer, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical adenoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In certain embodiments, the cancer is a solid tumor, such as a sarcoma, carcinoma, or lymphoma. In certain embodiments, the cancer is kidney cancer; hepatocellular carcinoma (HCC) or hepatoblastoma, or liver cancer; melanoma; breast cancer; colorectal carcinoma, or colorectal cancer; colon cancer; rectal cancer; anal cancer; lung cancer, such as non-small cell lung cancer (NSCLC) or small cell lung cancer (SCLC); ovarian cancer, ovarian epithelial cancer, ovarian carcinoma, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical carcinoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In certain embodiments, the cancer is selected from renal cell carcinoma, hepatocellular carcinoma (HCC), hepatoblastoma, colorectal carcinoma, colorectal cancer, colon cancer, rectal cancer, anal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, chondrosarcoma, anaplastic thyroid cancer, adrenocortical carcinoma, pancreatic cancer, pancreatic ductal carcinoma, pancreatic adenocarcinoma, glioma, brain cancer, neurofibromatosis-1 associated malignant peripheral nerve sheath tumors (MPNST), Waldenstrom's macroglobulinemia, or medulloblastoma.

In certain embodiments, the cancer is selected from hepatocellular carcinoma (HCC), hepatoblastoma, colon cancer, rectal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical carcinoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In certain embodiments, the cancer is hepatocellular carcinoma (HCC). In some embodiments, the cancer is hepatoblastoma. In some embodiments, the cancer is colon cancer. In some embodiments, the cancer is rectal cancer. In some embodiments, the cancer is ovarian cancer, or ovarian carcinoma. In some embodiments, the cancer is ovarian epithelial cancer. In some embodiments, the cancer is fallopian tube cancer. In some embodiments, the cancer is papillary serous cystadenocarcinoma. In some embodiments, the cancer is uterine papillary serous carcinoma (UPSC). In some embodiments, the cancer is hepatocholangiocarcinoma. In some embodiments, the cancer is soft tissue and bone synovial sarcoma. In some embodiments, the cancer is rhabdomyosarcoma. In some embodiments, the cancer is osteosarcoma. In some embodiments, the cancer is anaplastic thyroid cancer. In some embodiments, the cancer is adrenocortical carcinoma. In some embodiments, the cancer is pancreatic cancer, or pancreatic ductal carcinoma. In some embodiments, the cancer is pancreatic adenocarcinoma. In some embodiments, the cancer is glioma. In some embodiments, the cancer is malignant peripheral nerve sheath tumors (MPNST). In some embodiments, the cancer is neurofibromatosis-1 associated MPNST. In some embodiments, the cancer is Waldenstrom's macroglobulinemia. In some embodiments, the cancer is medulloblastoma.

In certain embodiments, the disorder is an autoimmune disease. In certain embodiments, the disorder is rheumatoid arthritis, psoriasis, Crohn's disease, inflammatory bowel disease, multiple sclerosis, systemic lupus erythematosus, Celiac Sprue, idiopathic thrombocytopenic thrombotic purpura, Sjogren's syndrome, scleroderma, ulcerative colitis, uveitis, Grave's disease, type I diabetes, polymyalgia rheumatic, alopecia, psoriasis, or vasculitis.

In certain embodiments, the disorder is a neurodegenerative disease. In certain embodiments, the disorder is Alzheimer's disease, Parkinson's disease, Lewy body disease, dementia, Huntingtin's disease, bipolar disorder, schizophrenia, an anxiety disorder, major depression, Gaucher disease, amyotrophic lateral sclerosis, olivopontocerebellar angiopathy, Batten's disease, prion, Creutzfeldt-Jakob Disease, primary progressive aphasia, Progressive Supranuclear Palsy, epilepsy, myasthenia gravis, neuropathy, or ataxia.

In certain embodiments, the disorder is a metabolic disease.

In certain embodiments, the disorder is pyruvate kinase deficiency, a glycogen storage disease, von Hippel-Lindau disease, achondrolasia, hypochondroplasia, Apert syndrome, Pfeiffer Syndrome, Crouzon syndrome, Jackson-Weiss syndrome, Muenke syndrome, polycystic kidney disease, beta-thalassemia, cystic fibrosis, Kennedy's disease, or Noonan syndrome.

The phosphorylation state of various proteins is understood to be linked to various medical disorders. Reducing the extent to which such a protein is phosphorylated can provide medical benefits. Exemplary medical disorders believed to be connected to protein phosphorylation are described below for exemplary proteins.

### Tau

Hyperphosphorylated Tau in the brain of patients with tauopathy results from an imbalance of kinase and phosphatase activity as well as mutations in the MAPT gene that encodes Tau (see, e.g., Takashima, Akihiko in “Tauopathies and tau oligomers,” Journal of Alzheimer's Disease 37.3 (2013): 565-568). Pathological phosphorylation of Tau causes it to fibrillize, disrupt intracellular trafficking, and seeds pathology in neighboring cells (see, e.g., Schneider, A., et al. in “Phosphorylation that detaches tau protein from microtubules (Ser262, Ser214) also protects it against aggregation into Alzheimer paired helical filaments,” Biochemistry 38.12 (1999): 3549-3558). The accumulation of phosphorylated Tau correlates with disease progression and dephosphorylation of Tau is believed to have therapeutic value in the treatment of tauopathy including Alzheimer's disease, frontotemporal dementia with parkinsonism-17, progressive supranuclear palsy, corticobasal degeneration, Pick's disease, globular glial tauopathy, and argyrophillic grain disease. For additional information, see, e.g., G\acute{m}ez-Isla, Teresa, et al. in “Neuronal loss correlates with but exceeds neurofibrillary tangles in Alzheimer's disease,” Annals of Neurology: Official Journal of the American Neurological Association and the Child Neurology Society 41.1 (1997): 17-24.

### InsR

Insulin receptor is phosphorylated on several residues in the activation loop of the kinase domain. Phosphorylation of tyrosine residues in the activation loop occurs in response to insulin binding and activates signaling (see, e.g., Petersen, Max C., and Gerald I. Shulman in “Mechanisms of insulin action and insulin resistance,” Physiological reviews 98.4 (2018): 2133-222). Dephosphorylation of these tyrosine residues and decreased insulin receptor activity are believed to have therapeutic benefit in the treatment of many oncology indications. Phosphorylation of a threonine residue in the activation loop of insulin receptor has been shown to inhibit insulin receptor activity. Dephosphorylation of inhibitory phosphorylation sites in insulin receptor may be beneficial in the treatment of insulin-resistance.

### IRS1/IRS2

Insulin receptor substrate 1 (IRS-1) and 2 (IRS-2) are downstream effectors of the insulin signaling pathway. The activity of IRS-1/2 is regulated by the balance of activating phosphorylation on tyrosine residues and inhibitory phosphorylation of serine and threonine residues (see, e.g., Hanger, Nancy J., et al. in “Insulin and metabolic stress stimulate multisite serine/threonine phosphorylation of insulin receptor substrate 1 and inhibit tyrosine phosphorylation,” Journal of Biological Chemistry 289.18 (2014): 12467-12484). Adipose and skeletal tissues from patients with type II diabetes mellitus demonstrate impaired insulin-stimulated phosphorylation of tyrosine residues in IRS1 as well as decreased total IRS1 protein (Gual, Philippe, Yannick Le Marchand-Brustel, and Jean-Francois Tanti in “Positive and negative regulation of insulin signaling through IRS-1 phosphorylation,” Biochimie 87.1 (2005): 99-109). Removal of repressive phosphorylation on serine and threonine residues is believed to increase insulin responsiveness and sensitivity and be of therapeutic value in the treatment of insulin-resistance.

### α-Synuclein

The abnormal accumulation of intraneuronal inclusions called Lewy bodies is the neuropathological hallmark of Parkinson's disease. These inclusions are primarily made of phosphorylated α-synuclein. In the brains of healthy patients, less than 4% of all α-synuclein is phosphorylated (see, e.g., Oueslati, Abid in “Implication of alpha-synuclein phosphorylation at S129 in synucleinopathies: what have we learned in the last decade?,” Journal of Parkinson's disease 6.1 (2016): 39-51). Phosphorylation of α-synuclein may enhance the ability of the protein to aggregate into toxic species as well as impair clearance of the protein, leading to accumulation (see, e.g., Kosten, Jonas, et al. in “Efficient modification of alpha-synuclein serine 129 by protein kinase CK1 requires phosphorylation of tyrosine 125 as a priming event,” in ACS chemical neuroscience 5.12 (2014): 1203-1208). Dephosphorylation of α-synuclein may provide therapeutic benefit by inhibiting aggregation and permitting the clearance.

### Huntington

Huntington's disease is caused by an expanded polyglutamine tract in huntington protein (Htt). Mutant Htt is prone to aggregation and the toxicity of Htt is mediated by amino terminal fragments of the protein released following proteolytic cleavage. The aggregation and cleavage of Htt is regulated by phosphorylation of Htt at multiple residues (see, e.g., Warby, Simon C., et al. in “Phosphorylation of huntingtin reduces the accumulation of its nuclear fragments,” Molecular and Cellular Neuroscience 40.2 (2009): 121-127). Dephosphorylation of the residues regulating these processes is believed to provide therapeutic benefit in the treatment of Parkinson's disease.

### MEK (e.g., a MEK1 or MEK2)

Phosphorylated MEK protein (primarily on S218 and S222) is a hallmark of hyperactive MAPK signaling pathway observed in various cancers. The hyperphosphorylated state can be a consequence of activating mutations upstream of MEK or stimulation of the pathway by mitogens or growth factors, thus leading to activation of a downstream substrate of MEK, phospho-ERK. Genetic markers of hyperactive MEK may include EGFR, Ras, PI3K and RAF mutations. Blocking and reversing Mek phosphorylation is believed to inhibit the signaling pathway.

### ERK/ERK1/ERK2

Phosphorylation of residues T202/Y204 on Erk1 and T185/Y187 on Erk2 results in hyperactive MAPK pathway signaling. This pathway can be overactive in various cancers due to upstream genetic alterations in EGFR, Ras, PI3K and RAF, as well as upon stimulation by growth factors, mitogens and cytokines. Blocking and reversing Erk phosphorylation is believed to inhibit the signaling pathway.

### K-Ras

Phosphorylated K-Ras has been shown to be activated upon phosphorylation. The phosphorylated K-Ras may activate MAPK pathway by enhancing binding and activation of RAF kinases. Thus, dephosphorylation of K-Ras is believed to inhibit MAPK signaling pathway and ameliorate disease where hyperactive MAPK is observed. For additional information, see, e.g., Barceló C, Paco N, Morell M, Alvarez-Moya B, Bota-Rabassedas N, Jaumot M, Vilardell F, Capella G, Agell N. in “Phosphorylation at Ser-181 of oncogenic KRAS is required for tumor growth,” Cancer Res. 2014 Feb. 15; 74(4):1190-9.

RAF kinases

RAF kinases are recognized disease drivers in various oncology indications. Gain-of-function mutations and amplifications are observed in clinical samples in melanoma, Langerhans cell histiocytosis, lung cancer, colorectal cancer, polycythemia vera and other neoplastic diseases. Phosphorylation of BRAF and CRAF has been shown to occur upon mitogenic stimulation, thereby activating downstream MAPK signaling. Further, certain phosphorylation events on BRAF and CRAF have been associated with inhibitory activity. It is believed that removal of these inhibitory sites could hyperactivate RAF kinase and lead to synthetic lethality with hyperactivating mutations in the pathway, such as Ras V12 mutations. For additional information, see, e.g., Varga et al. in Sci Signal. 2017 Mar. 7; 10(469

### PI3K

Phosphoinositide 3-kinases (PI3Ks) are lipid kinases implicated in cancer growth, insulin signaling, memory and metabolism. It is believed that dephosphorylation of PI3K subunits P85 and P110 inhibits its downstream signaling.

### AKT (e.g., ATK1, AKT2, or AKT3)

AKT kinase is fully activated upon dual phosphorylation on S473 (by mTORC2) and T308 (by PDK1). Various malignancies have been attributed to hyperactive AKT kinase, either upon upstream activation, or direct gain-of-function mutations in AKT. Due in part to the foregoing, it is believe that dephosphorylation of AKT will diminish disease manifestations associated with this pathway. Further, selective dephosphorylation of one site over another enables selective inhibition of disease driver activity of AKT kinases.

Rsk (e.g. Rsk1, Rsk2, Rsk3, Rsk4)

Sequential and coordinated phosphorylation of RSK isoforms occurs in the MAPK pathway. Classically, ERK has been shown to phosphorylate RSK, but numerous other kinases have also been shown to phosphorylate RSK. In turn, autophosphorylation of RSK leads to further increase in its N-terminal kinase activity. Multitude of RSK substrates have been elucidated and the phosphorylated state of these substrates are speculated to enhance cell's tumorigenic potential. Due in part to the foregoing, it is believed that dephosphorylation of RSK results in inhibition of cell proliferation in the context of hyperactive MAPK.

### Pyruvate Kinase (PKLR)

The activity of pyruvate kinase activity is tightly controlled by its phosphorylation state, whereby the enzyme is inhibited by phosphorylation and activated upon dephosphorylation. Multitude of mutations in the PKLR gene can lead to low levels of pyruvate kinase activity, ultimately leading to pyruvate kinase deficiency. Due in part to the foregoing, activation of PKLR activity upon dephosphorylation is believed to activate the enzyme and ameliorate symptoms associated with pyruvate kinase deficiency.

### FGFR1, FGFR2, FGFR3, FGFR4

Fibroblast growth factor signaling has been implicated in various cancers and skeletal development. It is believed that reversal of tyrosine phosphorylation events on FGFR will inhibit the signaling pathway, such that the downstream activation of FGFR scaffolding proteins is blocked.

### STAT3

STAT3 becomes transcriptionally active upon phosphorylation at tyrosine 705 or serine 727 residues. Hyperactive STAT3 has been implicated in various cancers and immunological indications. It is believed that STAT3 dephosphorylation will inhibit STAT3 dimerization and its transcriptional activity.

mTOR

mTOR is a central kinase involved in controlling cell growth upon inputs from various nutrient sensing pathways. The activity of mTOR is controlled by phosphorylation. Further, multiple mTOR complexes can exist where different phosphorylation sites are phosphorylated on mTOR. Due in part to the foregoing, it is believed that selective dephosphorylation of mTOR can specifically inactivate certain mTOR kinase species.

### BAD

BAD is a phosphor-protein that is inactivated upon phosphorylation. In its dephosphorylated state, BAD sequesters pro-apoptotic proteins such as Bcl-2, Bcl-xl and MCL-1. Several kinases have been shown to phosphorylate BAD, including Akt, PKA and RSK kinases. Due in part to the foregoing, dephosphorylated BAD is believed to relieve the anti-apoptotic signal and permit apoptosis to proceed in various neoplastic indications.

### GSK3

For full activity, GSK3-beta has to be phosphorylated on Serine 9 residue and GSK3-alpha on serine 21. Various kinases have been reported to be responsible for this phosphorylation event. In its active state, GSK3 can phosphorylate beta-catenin proteins, which contain a phospho-degron. Thus, active GSK3 leads to beta-catenin degradation. Due in part to the foregoing, it is believed that activation of GSK3 upon dephosphorylation will lead to degradation of beta-catenin. This is believed to be beneficial in treating various cancers, including colorectal cancers driven by beta-catenin accumulation.

### IKK

IκB kinase (IKK) is an enzyme complex that is involved in propagating the cellular response to inflammation, via the NF-kappaB signaling. In a phosphorylated state IKK is degraded and this degradation releases NF-kappaB and allows it to transclocate into the nucleus to turn on various inflammation related genes. Thus, dephosphorylation of IKK sequesters NF-kappaB in the cytosol and blocks the inflammatory response.

### BRD4

Epigenetic reader BRD4 plays a vital role in transcriptional regulation, cellular growth control, and cell-cycle progression, and it remains an important drug target for multiple malignancies. It has been demonstrated that the activated state of Brd4 in due to its hyperphosphorylation. Due in part to the foregoing, it is believed that selective dephosphorylation of Brd4 would inhibit only the cancer related functions of Brd4.

### Glycogen Synthase

GSK3 phosphorylation of glycogen synthase leads to inhibition of its enzymatic activity.

In the liver and muscle, dephosphorylated GS is active and converts UDP-glucose into UDP and glycogen. Thus, activation of GS upon dephosphorylation would enable the rapid conversion of glucose from tissues. It is believed that dephosphorylated GS provides a therapeutic benefit to patients with diabetes and hyperglycemia and glycogen storage disease type 0.

### SOS1

SOS1 phosphorylation on S1178 is observed upon feedback phosphorylation by Erk or RSK kinases. The phosphorylated state leads to decreased interaction with GRB2 and disrupts SOS1 membrane localization. Dephosphorylated SOS1 is expected to hyperactivate Ras-GTP and it has been shown that in the setting up mutated Ras, overactivation of the Ras signaling can lead to synthetic lethality.

### EGFR

EGFR is a receptor tyrosine kinase and its phosphorylation state depends on extracellular mitogenic signals. Several EGFR kinase inhibitors have demonstrated clinical utility in various cancers, including lung cancer. It is believed that dephosphorylation of EGFR will inhibit the downstream MAPK pathway and provide clinical benefit to patients whose cancer is dependent on EGFR signaling.

The efficacy of compounds described herein in treating diseases described herein may be evaluated using assays described in the literature that are predictive of efficacy in treating the disease. Exemplary assays described in the literature include in vitro cell-based assays in which test compound is applied to cancer cells, and the cancer cells are then monitored for cell death. The methods of the invention comprise administering to the subject a therapeutically effective amount of at least one compound of the invention, which is optionally formulated in a pharmaceutical composition. In certain embodiments, the method further comprises administering to the subject an additional therapeutic agent that treats or prevents the disease or disorder contemplated herein.

In certain embodiments, administering the compound of the invention to the subject allows for administering a lower dose of the additional therapeutic agent as compared to the dose of the additional therapeutic agent alone that is required to achieve similar results in treating or preventing the disease or disorder contemplated herein. For example, in certain embodiments, the compound of the invention enhances the therapeutic activity of the additional therapeutic compound, thereby allowing for a lower dose of the additional therapeutic compound to provide the same effect.

In certain embodiments, the compound of the invention and the therapeutic agent are co-administered to the subject. In other embodiments, the compound of the invention and the therapeutic agent are coformulated and co-administered to the subject.

In certain embodiments, the subject is a mammal. In other embodiments, the mammal is a human. The invention also includes a method of dephosphorylating a target protein having a phosphate group. The method comprises exposing the target protein to a compound described herein (e.g., a compound of Formula (I)), to thereby dephosphorylate the target protein. In certain embodiments, the target protein is a target protein listed in Table I-1.

The invention further provides methods of measuring dephosphorylation of phospho-proteins, as described herein.

General Protocol to Measure Dephosphorylation of Phospho-Proteins, Including Halo Tag Fusions

Dephosphorylation of phospho-proteins can be measured according to the following protocol. Cells are purchased from ATCC and cultured in media (e.g., RPMI-1640 media supplemented with 10% FBS). Vehicle and test compound treatments (25 μM, 2.5 μM and 0.25 μM) are performed in 12-well plates for 2 hours. Cells are harvested and lysed in buffer (e.g., RIPA buffer (50 mM Tris pH8, 150 mM NaCl, 1% Tx-100, 0.1% SDS and 0.5% sodium deoxycholate)) supplemented with protease and phosphatase inhibitors. Lysates are clarified at 16,000 g for 10 minutes and supernatants are separated by SDS-PAGE. Immunoblotting is performed using a standard protocol using a phospho-specific antibody. Signal intensity for bands may be imaged on LiCor Odyssey imager.

Establishment of Stable HaloTag-Fusion Cell Lines

HEK-293 cells (ATCC) are transfected with HaloTag fusion DNA constructs containing puromycin selectable marker. Stable cell polyclonal lines expressing fusion proteins are selected in 1 μg/mL of puromycin. The expression and phosphorylation status of the fusion protein may be determined using the Western Protocol with appropriate antibodies directed towards the HaloTag fusion partner.

Western Protocol to Measure Dephosphorylation of p-TBK1

Dephosphorylation of p-TBK1 may be measured using the following procotol. Panc02.13 or THP-1 cells are purchased from ATCC and cultured in RPMI-1640 media, supplemented with 10% FBS. Poly I:C agonist (Invivogen) is added to the cells 1 hr before drug treatment. Vehicle and test compound treatments (25 μM, 2.5 μM and 0.25 μM) are performed in 12-well plates for 2 hours. Cells are harvested and lysed in RIPA buffer (50 mM Tris pH8, 150 mM NaCl, 1% Tx-100, 0.1% SDS and 0.5% sodium deoxycholate) supplemented with protease and phosphatase inhibitors. Lysates are clarified at 16,000 g for 10 minutes and supernatants were separated by SDS-PAGE. Immunoblotting is performed using standard protocols, with antibodies for p-TBK1 (Cell Signaling, #5483) and total TBK1 (Cell Signaling, #38066, #51877 or #3504). The signal intensity for bands may be imaged on a LiCor Odyssey imager.

Measurement of AKT Phospho S473 and T308 Using Alpha SureFire Kit (Perkin Elmer)—

Phosphorylation of S473 and T308 in AKT may be measured using the following protocol. PC3 or HEK-293 cells are cultured as suggested by ATCC. Vehicle and test compound treatments (25 μM, 2.5 μM and 0.25 μM) are performed in 96-well plates for 2 hours. Media is aspirated and the cells are lysed in 70 μL of lysis buffer (Perkin Elmer SureFire). Plates are agitated gently for 10 minutes at 400 rpm.

### Add Acceptor Mix to Lysates:

Transfer 10 πL of lysate to white 384 well Optiplate (Perkin-Elmer #6007290)
Add certain volumes of PC3 and HEK293 lysates to appropriate wells
Add 10 μL positive control to appropriate wells
Add 5 μL of appropriate Acceptor mix to each well (prepare as suggested in SureFire protocol)
Incubate for 1 hour at RT

### Add Donor Mix:

Donor mix is prepared within 15 minutes before Acceptor mix incubation is done; the Donor mix is used within 30 minutes of mixing.
Steps performed on Donor beads should be performed under low light conditions
Vortex Donor beads (to get beads into solution) and spin down briefly to collect solution at bottom of tube
Add 5 μL of appropriate mix to appropriate wells of 384 well Optiplate and cover with an aluminum plate seal
Incubate for 1 hour at RT
Spin down plates if necessary

### Read Plate:

Optimized protocol is called Alphalisa
   excitation is 680 for donor bead
   emission is 615 and 545 for pAKT and total AKT respectively
   Mirror need is AlphaPlex Dual Tb-Eu (#2102-5900)
   Filter needed is AlphaPlex Tb (#2100-5930)
   If insufficient signal is observed, then cover plate and keep in dark at RT O/N and re-read next day.

### Combination Therapies

The compounds useful within the methods of the invention may be used in combination with one or more additional therapeutic agents useful for treating any disease or disorder contemplated herein. These additional therapeutic agents may comprise compounds that are commercially available or synthetically accessible to those skilled in the art. These additional therapeutic agents are known to treat, prevent, or reduce the symptoms, of a disease or disorder contemplated herein.

A synergistic effect may be calculated, for example, using suitable methods such as, for example, the Sigmoid-Eₘₐₓ equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

In certain embodiments, the compound is administered in combination with a second therapeutic agent having activity against cancer. In certain embodiments, the second therapeutic agent is mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma, colony stimulating factor-1, colony stimulating factor-2, denileukin diftitox, interleukin-2, and leutinizing hormone releasing factor.

In certain embodiments, the second therapeutic agent is an mTOR inhibitor, which inhibits cell proliferation, angiogenesis and glucose uptake. Approved mTOR inhibitors useful in the present invention include everolimus (Afinitor®, Novartis); temsirolimus (Torisel®, Pfizer); and sirolimus (Rapamune®, Pfizer).

In certain embodiments, the second therapeutic agent is a Poly ADP ribose polymerase (PARP) inhibitor. Approved PARP inhibitors useful in the present invention include olaparib (Lynparza®, AstraZeneca); rucaparib (Rubraca®, Clovis Oncology); and niraparib (Zejula®, Tesaro). Other PARP inhibitors being studied which may be used in the present invention include talazoparib (MDV3800/BMN 673/LT00673, Medivation/Pfizer/Biomarin); veliparib (ABT-888, AbbVie); and BGB-290 (BeiGene, Inc.).

In certain embodiments, the second therapeutic agent is a phosphatidylinositol 3 kinase (PI3K) inhibitor. Approved PI3K inhibitors useful in the present invention include idelalisib (Zydelig®, Gilead). Other PI3K inhibitors being studied which may be used in the present invention include alpelisib (BYL719, Novartis); taselisib (GDC-0032, Genentech/Roche); pictilisib (GDC-0941, Genentech/Roche); copanlisib (BAY806946, Bayer); duvelisib (formerly IPI-145, Infinity Pharmaceuticals); PQR309 (Piqur Therapeutics, Switzerland); and TGR1202 (formerly RP5230, TG Therapeutics).

In certain embodiments, the second therapeutic agent is a proteasome inhibitor. Approved proteasome inhibitors useful in the present invention include bortezomib (Velcade®, Takeda); carfilzomib (Kyprolis®, Amgen); and ixazomib (Ninlaro®, Takeda).

In certain embodiments, the second therapeutic agent is a histone deacetylase (HDAC) inhibitor. Approved HDAC inhibitors useful in the present invention include vorinostat (Zolinza®, Merck); romidepsin (Istodax®, Celgene); panobinostat (Farydak®, Novartis); and belinostat (Beleodaq®, Spectrum Pharmaceuticals). Other HDAC inhibitors being studied which may be used in the present invention include entinostat (SNDX-275, Syndax Pharmaceuticals) (NCT00866333); and chidamide (Epidaza®, HBI-8000, Chipscreen Biosciences, China).

In certain embodiments, the second therapeutic agent is a CDK inhibitor, such as a CDK 4/6 inhibitor. Approved CDK4/6 inhibitors useful in the present invention include palbociclib (Ibrance®, Pfizer); and ribociclib (Kisqali®, Novartis). Other CDK4/6 inhibitors being studied which may be used in the present invention include abemaciclib (Ly2835219, Eli Lilly); and trilaciclib (G1T28, G1 Therapeutics).

In certain embodiments, the second therapeutic agent is an indoleamine (2,3)-dioxygenase (IDO) inhibitor. IDO inhibitors being studied which may be used in the present invention include epacadostat (INCB024360, Incyte); indoximod (NLG-8189, NewLink Genetics Corporation); capmanitib (INC280, Novartis); GDC-0919 (Genentech/Roche); PF-06840003 (Pfizer); BMS:F001287 (Bristol-Myers Squibb); Phy906/KD108 (Phytoceutica); and an enzyme that breaks down kynurenine (Kynase, Kyn Therapeutics).

In certain embodiments, the second therapeutic agent is a growth factor antagonist, such as an antagonist of platelet-derived growth factor (PDGF), or epidermal growth factor (EGF) or its receptor (EGFR). Approved PDGF antagonists which may be used in the present invention include olaratumab (Lartruvo®; Eli Lilly). Approved EGFR antagonists which may be used in the present invention include cetuximab (Erbitux®, Eli Lilly); necitumumab (Portrazza®, Eli Lilly), panitumumab (Vectibix®, Amgen); and osimertinib (targeting activated EGFR, Tagrisso®, AstraZeneca).

In certain embodiments, the second therapeutic agent is an aromatase inhibitor. Approved aromatase inhibitors which may be used in the present invention include exemestane (Aromasin®, Pfizer); anastazole (Arimidex®, AstraZeneca) and letrozole (Femara®, Novartis).

In certain embodiments, the second therapeutic agent is an antagonist of the hedgehog pathway. Approved hedgehog pathway inhibitors which may be used in the present invention include sonidegib (Odomzo®, Sun Pharmaceuticals); and vismodegib (Erivedge®, Genentech), both for treatment of basal cell carcinoma.

In certain embodiments, the second therapeutic agent is a folic acid inhibitor. Approved folic acid inhibitors useful in the present invention include pemetrexed (Alimta®, Eli Lilly).

In certain embodiments, the second therapeutic agent is a CC chemokine receptor 4 (CCR4) inhibitor. CCR4 inhibitors being studied that may be useful in the present invention include mogamulizumab (Poteligeo®, Kyowa Hakko Kirin, Japan).

In certain embodiments, the second therapeutic agent is an isocitrate dehydrogenase (IDH) inhibitor. IDH inhibitors being studied which may be used in the present invention include AG120 (Celgene; NCT02677922); AG221 (Celgene, NCT02677922; NCT02577406); BAY1436032 (Bayer, NCT02746081); IDH305 (Novartis, NCT02987010).

In certain embodiments, the second therapeutic agent is an arginase inhibitor. Arginase inhibitors being studied which may be used in the present invention include AEB 1102 (pegylated recombinant arginase, Aeglea Biotherapeutics), which is being studied in Phase 1 clinical trials for acute myeloid leukemia and myelodysplastic syndrome (NCT02732184) and solid tumors (NCT02561234); and CB-1158 (Calithera Biosciences).

In certain embodiments, the second therapeutic agent is a glutaminase inhibitor. Glutaminase inhibitors being studied which may be used in the present invention include CB-839 (Calithera Biosciences).

In certain embodiments, the second therapeutic agent is an antibody that binds to tumor antigens, that is, proteins expressed on the cell surface of tumor cells. Approved antibodies that bind to tumor antigens which may be used in the present invention include rituximab (Rituxan®, Genentech/BiogenIdec); ofatumumab (anti-CD20, Arzerra®, GlaxoSmithKline); obinutuzumab (anti-CD20, Gazyva®, Genentech), ibritumomab (anti-CD20 and Yttrium-90, Zevalin®, Spectrum Pharmaceuticals); daratumumab (anti-CD38, Darzalex®, Janssen Biotech), dinutuximab (anti-glycolipid GD2, Unituxin®, United Therapeutics); trastuzumab (anti-HER2, Herceptin®, Genentech); ado-trastuzumab emtansine (anti-HER2, fused to emtansine, Kadcyla®, Genentech); and pertuzumab (anti-HER2, Perjeta®, Genentech); and brentuximab vedotin (anti-CD30-drug conjugate, Adcetris®, Seattle Genetics).

In certain embodiments, the second therapeutic agent is a topoisomerase inhibitor. Approved topoisomerase inhibitors useful in the present invention include irinotecan (Onivyde®, Merrimack Pharmaceuticals); topotecan (Hycamtin®, GlaxoSmithKline). Topoisomerase inhibitors being studied which may be used in the present invention include pixantrone (Pixuvri®, CTI Biopharma).

In certain embodiments, the second therapeutic agent is a nucleoside inhibitor, or other therapeutic that interfere with normal DNA synthesis, protein synthesis, cell replication, or will otherwise inhibit rapidly proliferating cells. Such nucleoside inhibitors or other therapeutics include trabectedin (guanidine alkylating agent, Yondelis®, Janssen Oncology), mechlorethamine (alkylating agent, Valchlor®, Aktelion Pharmaceuticals); vincristine (Oncovin®, Eli Lilly; Vincasar®, Teva Pharmaceuticals; Marqibo®, Talon Therapeutics); temozolomide (prodrug to alkylating agent 5-(3-methyltriazen-1-yl)-imidazole-4-carboxamide (MTIC) Temodar®, Merck); cytarabine injection (ara-C, antimetabolic cytidine analog, Pfizer); lomustine (alkylating agent, CeeNU®, Bristol-Myers Squibb; Gleostine®, NextSource Biotechnology); azacitidine (pyrimidine nucleoside analog of cytidine, Vidaza®, Celgene); omacetaxine mepesuccinate (cephalotaxine ester) (protein synthesis inhibitor, Synribo®; Teva Pharmaceuticals); asparaginase Erwinia chrysanthemi (enzyme for depletion of asparagine, Elspar®, Lundbeck; Erwinaze®, EUSA Pharma); eribulin mesylate (microtubule inhibitor, tubulin-based antimitotic, Halaven®, Eisai); cabazitaxel (microtubule inhibitor, tubulin-based antimitotic, Jevtana®, Sanofi-Aventis); capacetrine (thymidylate synthase inhibitor, Xeloda®, Genentech); bendamustine (bifunctional mechlorethamine derivative, believed to form interstrand DNA cross-links, Treanda®, Cephalon/Teva); ixabepilone (semi-synthetic analog of epothilone B, microtubule inhibitor, tubulin-based antimitotic, Ixempra®, Bristol-Myers Squibb); nelarabine (prodrug of deoxyguanosine analog, nucleoside metabolic inhibitor, Arranon®, Novartis); clorafabine (prodrug of ribonucleotide reductase inhibitor, competitive inhibitor of deoxycytidine, Clolar®, Sanofi-Aventis); and trifluridine and tipiracil (thymidine-based nucleoside analog and thymidine phosphorylase inhibitor, Lonsurf®, Taiho Oncology).

In certain embodiments, the second therapeutic agent is a platinum-based therapeutic, also referred to as platins. Platins cause cross-linking of DNA, such that they inhibit DNA repair and/or DNA synthesis, mostly in rapidly reproducing cells, such as cancer cells. Approved platinum-based therapeutics which may be used in the present invention include cisplatin (Platinol®, Bristol-Myers Squibb); carboplatin (Paraplatin®, Bristol-Myers Squibb; also, Teva; Pfizer); oxaliplatin (Eloxitin® Sanofi-Aventis); and nedaplatin (Aqupla®, Shionogi). Other platinum-based therapeutics which have undergone clinical testing and may be used in the present invention include picoplatin (Poniard Pharmaceuticals); and satraplatin (JM-216, Agennix).

In certain embodiments, the second therapeutic agent is a taxane compound, which causes disruption of microtubules, which are essential for cell division. Approved taxane compounds which may be used in the present invention include paclitaxel (Taxol®, Bristol-Myers Squibb), docetaxel (Taxotere®, Sanofi-Aventis; Docefrez®, Sun Pharmaceutical), albumin-bound paclitaxel (Abraxane®; Abraxis/Celgene), and cabazitaxel (Jevtana®, Sanofi-Aventis). Other taxane compounds which have undergone clinical testing and may be used in the present invention include SID530 (SK Chemicals, Co.) (NCT00931008).

In certain embodiments, the second therapeutic agent is an inhibitor of anti-apoptotic proteins, such as BCL-2. Approved anti-apoptotics which may be used in the present invention include venetoclax (Venclexta®, AbbVie/Genentech); and blinatumomab (Blincyto®, Amgen). Other therapeutic agents targeting apoptotic proteins which have undergone clinical testing and may be used in the present invention include navitoclax (ABT-263, Abbott), a BCL-2 inhibitor (NCT02079740).

In certain embodiments, the second therapeutic agent is a selective estrogen receptor modulator (SERM), which interferes with the synthesis or activity of estrogens. Approved SERMs useful in the present invention include raloxifene (Evista®, Eli Lilly).

In certain embodiments, the second therapeutic agent is an inhibitor of interaction between the two primary p53 suppressor proteins, MDMX and MDM2. Inhibitors of p53 suppression proteins being studied which may be used in the present invention include ALRN-6924 (Aileron), a stapled peptide that equipotently binds to and disrupts the interaction of MDMX and MDM2 with p53. ALRN-6924 is currently being evaluated in clinical trials for the treatment of AML, advanced myelodysplastic syndrome (MDS) and peripheral T-cell lymphoma (PTCL) (NCT02909972; NCT02264613).

In certain embodiments, the second therapeutic agent is an inhibitor of transforming growth factor-beta (TGF-beta or TGFβ). Inhibitors of TGF-beta proteins being studied which may be used in the present invention include NIS793 (Novartis), an anti-TGF-beta antibody being tested in the clinic for treatment of various cancers, including breast, lung, hepatocellular, colorectal, pancreatic, prostate and renal cancer (NCT 02947165). In some embodiments, the inhibitor of TGF-beta proteins is fresolimumab (GC 1008; Sanofi-Genzyme), which is being studied for melanoma (NCT00923169); renal cell carcinoma (NCT00356460); and non-small cell lung cancer (NCT02581787). Additionally, in some embodiments, the additional therapeutic agent is a TGF-beta trap, such as described in Connolly et al. (2012) Int'l J. Biological Sciences 8:964-978. One therapeutic compound currently in clinical trials for treatment of solid tumors is M7824 (Merck KgaA-formerly MSB0011459X), which is a bispecific, anti-PD-L1/TGFβ trap compound (NCT02699515); and (NCT02517398). M7824 is comprised of a fully human IgG1 antibody against PD-L1 fused to the extracellular domain of human TGF-beta receptor II, which functions as a TGFβ “trap.”

In certain embodiments, the second therapeutic agent is a cancer vaccine. In some embodiments, the cancer vaccine is selected from sipuleucel-T (Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (Imlygic®, BioVex/Amgen, previously known as T-VEC), a genetically modified oncolytic viral therapy approved for treatment of unresectable cutaneous, subcutaneous and nodal lesions in melanoma. In some embodiments, the additional therapeutic agent is selected from an oncolytic viral therapy such as pexastimogene devacirepvec (PexaVec/JX-594, SillaJenlformerly Jennerex Biotherapeutics), a thymidine kinase-(TK-) deficient vaccinia virus engineered to express GM-CSF, for hepatocellular carcinoma (NCT02562755) and melanoma (NCT00429312); pelareorep (Reolysin®, Oncolytics Biotech), a variant of respiratory enteric orphan virus (reovirus) which does not replicate in cells that are not RAS-activated, in numerous cancers, including colorectal cancer (NCT01622543); prostate cancer (NCT01619813); head and neck squamous cell cancer (NCT01166542); pancreatic adenocarcinoma (NCT00998322); and non-small cell lung cancer (NSCLC) (NCT 00861627); enadenotucirev (NG-348, PsiOxus, formerly known as ColoAd1), an adenovirus engineered to express a full length CD80 and an antibody fragment specific for the T-cell receptor CD3 protein, in ovarian cancer (NCT02028117); metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676); and peritoneal disease, colorectal cancer or ovarian cancer (NCT02963831); GL-ONC1 (GLV-1h68/GLV-h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260); fallopian tube cancer, ovarian cancer (NCT 02759588); or CG0070 (Cold Genesys), an adenovirus engineered to express GM-CSF, in bladder cancer (NCT02365818).

In certain embodiments, the second therapeutic agent is an immune checkpoint inhibitor selected from a PD-1 antagonist, a PD-L1 antagonist, or a CTLA-4 antagonist. In some embodiments, a compound disclosed herein or a pharmaceutically acceptable salt thereof is administered in combination with nivolumab (anti-PD-1 antibody, Opdivo®, Bristol-Myers Squibb); pembrolizumab (anti-PD-1 antibody, Keytruda®, Merck); ipilimumab (anti-CTLA-4 antibody, Yervoy®, Bristol-Myers Squibb); durvalumab (anti-PD-L1 antibody, Imfinzi®, AstraZeneca); or atezolizumab (anti-PD-L1 antibody, Tecentriq®, Genentech). Other immune checkpoint inhibitors suitable for use in the present invention include REGN2810 (Regeneron), an anti-PD-1 antibody tested in patients with basal cell carcinoma (NCT03132636); NSCLC (NCT03088540); cutaneous squamous cell carcinoma (NCT02760498); lymphoma (NCT02651662); and melanoma (NCT03002376); pidilizumab (CureTech), also known as CT-011, an antibody that binds to PD-1, in clinical trials for diffuse large B-cell lymphoma and multiple myeloma; avelumab (Bavencio®, Pfizer/Merck KGaA), also known as MSB0010718C), a fully human IgG1 anti-PD-L1 antibody, in clinical trials for non-small cell lung cancer, Merkel cell carcinoma, mesothelioma, solid tumors, renal cancer, ovarian cancer, bladder cancer, head and neck cancer, and gastric cancer; and PDR001 (Novartis), an inhibitory antibody that binds to PD-1, in clinical trials for non-small cell lung cancer, melanoma, triple negative breast cancer and advanced or metastatic solid tumors. Tremelimumab (CP-675, 206; Astrazeneca) is a fully human monoclonal antibody against CTLA-4 that has been in studied in clinical trials for a number of indications, including: mesothelioma, colorectal cancer, kidney cancer, breast cancer, lung cancer and non-small cell lung cancer, pancreatic ductal adenocarcinoma, pancreatic cancer, germ cell cancer, squamous cell cancer of the head and neck, hepatocellular carcinoma, prostate cancer, endometrial cancer, metastatic cancer in the liver, liver cancer, large B-cell lymphoma, ovarian cancer, cervical cancer, metastatic anaplastic thyroid cancer, urothelial cancer, fallopian tube cancer, multiple myeloma, bladder cancer, soft tissue sarcoma, and melanoma. AGEN-1884 (Agenus) is an anti-CTLA4 antibody that is being studied in Phase 1 clinical trials for advanced solid tumors (NCT02694822).

### Administration/Dosage/Formulations

The regimen of administration may affect what constitutes an effective amount. The therapeutic formulations may be administered to the subject either prior to or after the onset of a disease or disorder contemplated herein. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat a disease or disorder contemplated herein. An effective amount of the therapeutic compound necessary to achieve a therapeutic effect may vary according to factors such as the state of the disease or disorder in the patient; the age, sex, and weight of the patient; and the ability of the therapeutic compound to treat a disease or disorder contemplated herein. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a cancer in a patient.

In certain embodiments, the compositions of the invention are formulated using one or more pharmaceutically acceptable excipients or carriers. In certain embodiments, the pharmaceutical compositions of the invention comprise a therapeutically effective amount of a compound of the invention and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions of the invention are administered to the patient in dosages that range from one to five times per day or more. In other embodiments, the compositions of the invention are administered to the patient in range of dosages that include, but are not limited to, once every day, every two days, every three days to once a week, and once every two weeks. It is readily apparent to one skilled in the art that the frequency of administration of the various combination compositions of the invention varies from individual to individual depending on many factors including, but not limited to, age, disease or disorder to be treated, gender, overall health, and other factors. Thus, the invention should not be construed to be limited to any particular dosage regime and the precise dosage and composition to be administered to any patient is determined by the attending physical taking all other factors about the patient into account.

Compounds of the invention for administration may be in the range from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments there between.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of a disease or disorder contemplated herein.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions of the invention include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds of the invention may be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

### Parenteral Administration

For parenteral administration, the compounds of the invention may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as Ph Helv or similar alcohol.

### Additional Administration Forms

Additional dosage forms of this invention include dosage forms as described in U.S. Pat. Nos. 6,340,475; 6,488,962; 6,451,808; 5,972,389; 5,582,837; and 5,007,790. Additional dosage forms of this invention also include dosage forms as described in U.S. Patent Applications Nos. 20030147952; 20030104062; 20030104053; 20030044466; 20030039688; and 20020051820. Additional dosage forms of this invention also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer than the same amount of agent administered in bolus form.

For sustained release, the compounds may be formulated with a suitable polymer or hydrophobic material which provides sustained release properties to the compounds. As such, the compounds for use by the method of the invention may be administered in the form of micro particles, for example, by injection or in the form of wafers or discs by implantation.

In one embodiment of the invention, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound of the present invention depends on the age, sex and weight of the patient, the current medical condition of the patient and the progression of a disease or disorder contemplated herein in the patient being treated. The skilled artisan is able to determine appropriate dosages depending on these and other factors.

A suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the inhibitor of the invention is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced to a level at which the improved disease is retained. In certain embodiments, patients require intermittent treatment on a long-term basis upon any recurrence of symptoms and/or infection.

The compounds for use in the method of the invention may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

### Methods and Materials

### General Methods

All reactions were carried out under an atmosphere of dry nitrogen or argon. Glassware was oven-dried prior to use. Unless otherwise indicated, common reagents or materials were obtained from commercial sources and used without further purification. N,N-Diisopropylethylamine (DIPEA) was obtained anhydrous by distillation over potassium hydroxide. Tetrahydrofuran (THF), Dichloromethane (CH₂Cl₂), and dimethylformamide (DMF) was dried by a PURESOLV™ solvent drying system. PTLC refers to preparatory thin layer chromatographic separation. Abbreviations: HFIP (hexafluoroisopropanol), HEPES (4-(2-hydroxyethyl)-1-piperazineethanesulfonic acid. Flash column chromatography was performed using silica gel 60 (230-400 mesh). Analytical thin layer chromatography (TLC) was carried out on Merck silica gel plates with QF-254 indicator and visualized by UV or KMnO₄.

¹H and ¹³C NMR spectra were recorded on an Agilent DD₂ 500 (500 MHz ¹H; 125 MHz ¹³C) or Agilent DD₂ 600 (600 MHz ¹H; 150 MHz ¹³C) or Agilent DD₂ 400 (400 MHz ¹H; 100 MHz 13C) spectrometer at room temperature. Chemical shifts were reported in ppm relative to the residual CDCl₃ (δ 7.26 ppm ¹H; δ 77.0 ppm ¹³C), CD₃OD (δ 3.31 ppm ¹H; δ 49.00 ppm ¹³C), or d₆-DMSO (δ 2.50 ppm ¹H; δ 39.52 ppm ¹³C). NMR chemical shifts were expressed in ppm relative to internal solvent peaks, and coupling constants were measured in Hz. (bs=broad signal). In most cases, only peaks of the major rotamer are reported.

Mass spectra were obtained using Agilent 1100 series LC/MSD spectrometers.

Analytical HPLC analyses were carried out on 250×4.6 mm C-18 column using gradient conditions (10-100% B, flow rate=1.0 mL/min, 20 min) or as described in the LC-MS Method tables.

Unless indicated otherwise, preparative HPLC was carried out on 250×21.2 mm C-18 column using gradient conditions (10-100% B, flow rate=10.0 mL/min, 20 min). The eluents used were: solvent A (H₂O with 0.1% TFA) and solvent B (CH₃CN with 0.1% TFA). Final products were typically purified via reversed-phase HPLC, PTLC, or flash column chromatography.

LC-MS Method 01

| Instrument | | Agilent 1100 LC & Agilent G1956A | | |
|---|---|---|---|---|
| Software | | Agilent Chemstation Rev. B. 04.03[54] | | |
| HPLC | Column | Agilent ZORBAX 5 μm SB-Aq, 2.1*50 mm | | |
| | Mobile Phase | A: 0.0375% TFA in water (v/v) | | |
| | | B: 0.01875% TFA in Acetonitrile (v/v) | | |
| | | Time(min) | B(%) | Flow(mL/min) |
| | Gradient | 0.00 | 1 | 0.8 |
| | | 0.40 | 1 | 0.8 |
| | | 3.40 | 90 | 0.8 |
| | | 3.90 | 100 | 0.8 |
| | | 3.91 | 1 | 0.8 |
| | | 4.00 | 1 | 1.0 |
| | | 4.50 | 1 | 1.0 |

| | | |
|---|---|---|
| | Post time(min) | 0 |
| | Column Temp | 50° C. |
| | Detector | DAD |
| MS | Ionization source | ESI |
| | Drying Gas | N2 |
| | Drying Gas Flow | 10(L/min) |
| | Nebulizer Pressure | 40(psi) |
| | Drying Gas | 350° C. |
| | Temperature | |
| | Capillary Voltage | 2500(V) |
| | MS Polarity | Positive |
| | MS Mode | Scan |
| | Mass Range | 100-1500 |

LC-MS Method 10

| Instrument | | Agilent 1100 LC & Agilent G1956A | | |
|---|---|---|---|---|
| Software | | Agilent Chemstation Rev. B. 04.03[54] | | |
| HPLC | Column | Agilent ZORBAX 5 μm SB-Aq, 2.1*50 mm | | |
| | Mobile Phase | A: 0.0375% TFA in water (v/v) | | |
| | | B: 0.01875% TFA in Acetonitrile (v/v) | | |
| | | Time(min) | B(%) | Flow(mL/min) |
| | Gradient | 0.00 | 10 | 0.8 |
| | | 0.40 | 10 | 0.8 |
| | | 3.40 | 100 | 0.8 |
| | | 3.90 | 100 | 0.8 |
| | | 3.91 | 10 | 0.8 |
| | | 4.00 | 10 | 1.0 |
| | | 4.50 | 10 | 1.0 |

| | | |
|---|---|---|
| | Post time(min) | 0 |
| | Column Temp | 50° C. |
| | Detector | DAD |
| MS | Ionization source | ESI |
| | Drying Gas | N₂ |
| | Drying Gas Flow | 10(L/min) |
| | Nebulizer Pressure | 40(psi) |
| | Drying Gas | 350° C. |
| | Temperature | |
| | Capillary Voltage | 2500(V) |
| | MS Polarity | Positive |
| | MS Mode | Scan |
| | Mass Range | 100-1500 |

LC-MS Method 00

| Instrument | | Agilent 1100 LC & Agilent G1956A | | |
|---|---|---|---|---|
| Software | | Agilent Chemstation Rev. B. 04.03[54] | | |
| HPLC | Column | Agilent ZORBAX 5 μm SB-Aq, 2.1*50 mm | | |
| | Mobile Phase | A: 0.0375% TFA in water (v/v) | | |
| | | B: 0.01875% TFA in Acetonitrile (v/v) | | |
| | | Time(min) | B(%) | Flow(mL/min) |
| | Gradient | 0.00 | 0 | 0.6 |
| | | 0.40 | 0 | 0.6 |
| | | 3.40 | 80 | 0.6 |
| | | 3.90 | 100 | 0.6 |
| | | 3.91 | 0 | 0.6 |
| | | 4.00 | 0 | 1.0 |
| | | 4.50 | 0 | 1.0 |

| | | |
|---|---|---|
| | Post time(min) | 1.20 |
| | Column Temp | 50° C. |
| | Detector | DAD |
| MS | Ionization source | ESI |
| | Drying Gas | N2 |
| | Drying Gas Flow | 10(L/min) |
| | Nebulizer Pressure | 40(psi) |
| | Drying Gas | 350° C. |
| | Temperature | |
| | Capillary Voltage | 2500(V) Positive |
| | MS Polarity | Positive |
| | MS Mode | Scan |
| | Mass Range | 50-1500 |

LC-MS Method 25

| Instrument | | Agilent 1100 LC & Agilent G1956A | | |
|---|---|---|---|---|
| Software | | Agilent Chemstation Rev. B. 04.03[54] | | |
| HPLC | Column | Agilent ZORBAX 5 μm SB-Aq, 2.1*50 mm | | |
| | Mobile Phase | A: 0.0375% TFA in water (v/v) | | |
| | | B: 0.01875% TFA in Acetonitrile (v/v) | | |
| | | Time(min) | B(%) | Flow(mL/min) |
| | Gradient | 0.00 | 25 | 0.8 |
| | | 0.40 | 25 | 0.8 |
| | | 3.40 | 100 | 0.8 |
| | | 3.90 | 100 | 0.8 |
| | | 3.91 | 25 | 0.8 |
| | | 4.00 | 25 | 1.0 |
| | | 4.50 | 25 | 1.0 |

| | | |
|---|---|---|
| | Post time(min) | 0 |
| | Column Temp | 50° C. |
| | Detector | DAD |
| MS | Ionization source | ESI |
| | Drying Gas | N2 |
| | Drying Gas Flow | 10(L/min) |
| | Nebulizer Pressure | 40(psi) |
| | Drying Gas | 350° C. |
| | Temperature | |
| | Capillary Voltage | 2500(V) Positive |
| | MS Polarity | Positive |
| | MS Mode | Scan |
| | Mass Range | 100-1500 |

### Example 1

### 1,2,3,4-tetrahydroacridine-9-carboxylic acid (Compound 1)

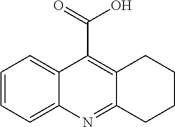 [SMILES: O=C(O)c1c2c(nc3ccccc13)CCCC2]

A solution of indoline-2,3-dione (1.00 g, 6.80 mmol) and cyclohexanone (0.667 g, 6.80 mmol) in potassium hydroxide (0.63 g, 11 mmol) in 20% aqueous ethanol (3 ml) was heated at 120° C. under microwave assisted conditions for 15 min. The reaction mixture was poured into a mixture of EtOAc:Hexane (1:1, 30 mL) and water (20 mL), then the aqueous layer was separated and the pH was adjusted to pH˜4.5 with an aqueous solution of oxalic acid (5%). The solid was collected by filtration, to give 0.77 g of product as a white solid (46% yield). ¹H NMR (400 MHz, DMSO-d₆) δ 7.92 (d, 1H), 7.80-7.63 (m, 2H), 7.63-7.43 (m, 1H), 3.05 (t, J=6.4 Hz, 2H), 2.89 (t, J=6.4 Hz, 2H), 2.06-1.75 (m, 4H). ¹³C NMR (151 MHz, DMSO-d₆) δ 168.65, 158.93, 145.54, 139.45, 129.05, 128.31, 126.51, 125.61, 124.31, 122.02, 33.30, 26.23, 22.22, 21.99. LC-MS (ESI); m/z [M+1]+; Calcd. for C₁₄H1₄NO₂, 228.1024. Found 228.23.

### 2-Chloro-N-(2-methoxyethyl)acetamide (compound 2)

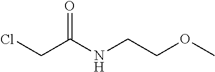

To a solution of 2-methoxyethanamine (1.03 g, 13.7 mmol) in a mixture of aqueous saturated solution of NaHCO₃ (50.0 mL) and DCM (50.0 mL) was added 2-chloroacetyl chloride (1.71 g, 15.1 mmol) dropwise at room temperature under vigorous stirring. The reaction mixture was transferred to a separation funnel, and the organic layer was separated, dried (Na₂SO₄) and evaporated under vacuum. The crude product was pure by NMR (>98% pure), 1.72 g as a yellow oil (82% yield). ¹H NMR (400 MHz, Chloroform-d) δ 6.92 (s, 1H), 4.06 (s, 2H), 3.49 (s, 4H), 3.38 (s, 3H). ¹³C NMR (151 MHz, Chloroform-d) δ 165.93, 70.70, 58.86, 42.59, 39.55. LC-MS (ESI); m/z [M+1]⁺; Calcd. for C₅H₁₁, ClNO₂, 152.0478. Found, 152.04.

### 2-((2-Methoxyethyl)amino)-2-oxoethyl 1,2,3,4-tetrahydroacridine-9-carboxylate (Compound 3)

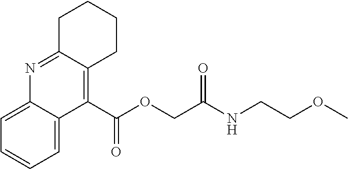 [SMILES: COCCNC(=O)COC(=O)c1c2c(nc3ccccc13)CCCC2]

To a solution of 1,2,3,4-tetrahydroacridine-9-carboxylic acid (1) (152 mg, 0.669 mmol) in DMF (5 mL) was added 2-chloro-N-(2-methoxyethyl)acetamide (2) (112 mg, 0.736 mmol), and then N,N-Diisopropylethylamine (0.175 mL, 1.00 mmol). The reaction mixture was stirred at 70° C. for 12 h (overnight), then the reaction mixture was diluted with EtOAc (30 mL), washed with brine/water (4×20 ml), dried (Na₂SO₄), and evaporated under vacuum. The crude product was purified by flash chromatography (SiO₂-12 g, gradient Hex:EtOAc, 6:4 to 100% EtOAc in 10 min) to give 173 mg of pure product as a white solid (68% yield). ¹H NMR (500 MHz, DMSO-d₆) δ 8.33 (s, 1H), 8.11 (d, J=8.3 Hz, 1H), 7.94 (d, J=8.4 Hz, 1H), 7.71 (t, J=7.4 Hz, 1H), 7.57 (t, J=7.3 Hz, 1H), 4.90 (s, 2H), 3.45-3.37 (m, 2H), 3.37-3.29 (m, 2H), 3.27 (s, 3H), 3.06 (t, J=5.5 Hz, 2H), 2.94 (t, J=5.3 Hz, 2H), 2.00-1.86 (m, 2H), 1.85-1.75 (m, 2H). ¹³C NMR (151 MHz, DMSO-d₆) δ 166.63, 166.25, 158.93, 145.59, 136.84, 129.21, 128.29, 127.13, 126.63, 124.77, 122.37, 70.52, 63.28, 63.28, 38.42, 33.33, 26.18, 22.17, 21.93. LC-MS (ESI); m/z [M+1]⁺; Calcd. C₁₉H₂₃N₂O₄, 343.1657. Found 343.16.

### 2-((2-methoxyethyl)amino)-2-oxoethyl 4-(4-hydroxybenzylidene)-1,2,3,4-tetrahydroacridine-9-carboxylate (compound 4)

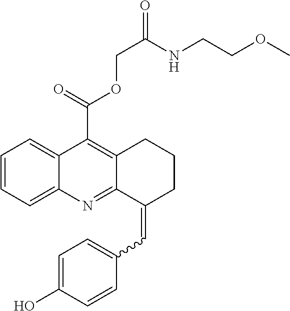 [SMILES: COCCNC(=O)COC(=O)c1c2c(nc3ccccc13)C(=Cc1ccc(O)cc1)CCC2]

To a solution of 4-hydroxybenzaldehyde (12.9 mg, 0.106 mmol) and [2-(2-methoxyethylamino)-2-oxo-ethyl]-1,2,3,4-tetrahydroacridine-9-carboxylate (3) (33.0 mg, 0.0964 mmol) in DMF (1.00 mL) was added Chlorotrimethylsilane (52.4 mg, 0.482 mmol), and the system was stirred at 100° C. for 12 h (overnight) in a sealed tube. When no more starting materials were observed by TLC (Hex:EtOAc, 1:1 and 1:9), the reaction mixture was diluted with a mixture of EtOAc (10 mL) and aqueous-saturated solution of NaHCO₃ (5 mL). The organic phase was washed with brine/water (4×5 mL), dried (Na₂SO₄), and evaporated under vacuum. The crude product was purified by PTLC (EtOAc 100%) to give 20 mg of pure product (46% yield). ¹H NMR (400 MHz, DMSO-d₆) δ 9.71 (s, 1H), 8.34 (t, J=4.6 Hz, 1H), 8.18-8.06 (m, 2H), 8.03 (d, J=8.5 Hz, 1H), 7.73 (t, J=7.6 Hz, 1H), 7.57 (t, J=7.6 Hz, 1H), 7.40 (d, J=7.8 Hz, 2H), 6.85 (d, J=7.4 Hz, 2H), 4.92 (s, 2H), 3.50-3.30 (m, 4H), 3.27 (s, 3H), 3.09-2.97 (m, 2H), 2.95 (t, J=6.3 Hz, 2H), 1.84 (p, J=5.9 Hz, 2H). ¹³C NMR (151 MHz, DMSO-d₆) δ 166.67, 166.29, 157.10, 153.92, 145.92, 136.82, 132.05, 131.49, 129.52, 129.46, 129.00, 127.77, 127.57, 126.82, 124.73, 122.54, 115.35, 70.54, 63.37, 57.95, 38.45, 27.72, 27.04, 21.93. LC-MS (ESI); m/z [M+1]⁺; Calcd. C₂₆H₂₇N₂O₅, 447.1919. Found 389.19.

### tert-Butyl (1,2,3,4-tetrahydroacridine-9-carbonyl)glycinate (compound 5)

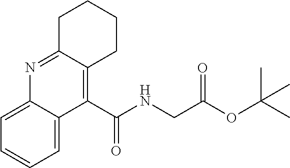 [SMILES: CC(C)(C)OC(=O)CNC(=O)c1c2c(nc3ccccc13)CCCC2]

To a solution of 1,2,3,4-tetrahydroacridine-9-carboxylic acid (1) (48.4 mg, 0.213 mmol), tert-butyl 2-aminoacetate; hydrochloride (39.3 mg, 0.234 mmol), and Triethylamine (0.148 mL, 1.06 mmol) in DMF (2 mL) was added HATU (89.1 mg, 0.234 mmol) at room temperature. The reaction was stirred at the same temperature for 12 h (overnight). The reaction mixture was diluted with EtOAc (10 mL) and washed with water/brine (1:1, 3×10 mL). The organic phase was dried (Na₂SO₄) and evaporated under vacuum. The crude product was purified by PTLC (DCM:MeOH:NH₄OH, 90:9:1) to give 48.4 mg of pure product (67%). ¹H NMR (400 MHz, DMSO-d₆) δ 9.08 (t, J=5.4 Hz, 1H), 8.06-7.79 (m, 2H), 7.68 (t, J=7.5 Hz, 1H), 7.53 (t, J=7.5 Hz, 1H), 4.01 (s, 2H), 3.05 (t, J=6.3 Hz, 2H), 2.97-2.80 (m, 2H), 2.00-1.71 (m, 4H), 1.49 (s, 9H). ¹³C NMR (101 MHz, DMSO-d₆) δ 168.75, 167.03, 158.77, 145.68, 141.38, 128.83, 128.17, 126.24, 126.02, 124.92, 123.20, 81.02, 41.65, 33.37, 27.82, 25.93, 22.43, 22.09. LC-MS (ESI); m/z [M+1]⁺; Calcd. C₂₀H₂₅N₂O₃, 341.1865. Found 341.18.

### (1,2,3,4-Tetrahydroacridine-9-carbonyl)glycine (compound 6)

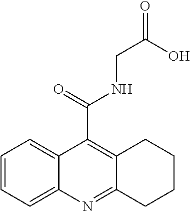 [SMILES: O=C(O)CNC(=O)c1c2c(nc3ccccc13)CCCC2]

A solution of tert-butyl (1,2,3,4-tetrahydroacridine-9-carbonyl)glycinate (5) (35.0 mg, 0.103 mmol) in a mixture of TFA (1.50 mL, 20.2 mmol) and Dichloromethane (3.00 mL) was stirred for 1.5 h (about 90% conversion by TLC). Then the solvent was removed under vacuum (20 min) and the crude product was dried under high vacuum for 20 min. The crude product was used in the next step without any further purification (quantitative yield). LC-MS (ESI); m/z: [M+H]+ Calcd. For C₁₆H₁₇N₂O₃, 285.1239. Found 285.12.

### N-(2-((2-methoxyethyl)amino)-2-oxoethyl)-1,2,3,4-tetrahydroacridine-9-carboxamide (Compound 7)

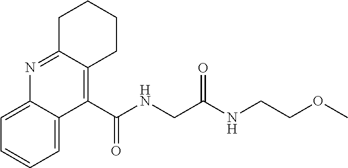 [SMILES: COCCNC(=O)CNC(=O)c1c2c(nc3ccccc13)CCCC2]

To a solution of 2-(1,2,3,4-tetrahydroacridine-9-carbonylamino)acetic acid (6) (29.2 mg, 0.103 mmol) in DMF (2 mL) was added 2-methoxyethanamine (11.6 mg, 0.154 mmol) and Triethylamine (0.0716 mL, 0.514 mmol) at room temperature. Then HATU (43.0 mg, 0.113 mmol) was added at the same temperature. The reaction was stirred at room temperature overnight (12 h). The reaction mixture was diluted with EtOAc (10 mL) and washed with water/brine (1:1, 3×10 mL), then the organic phase was dried (Na₂SO₄, and evaporated under vacuum. Crude product was purified by PTLC (DCM:MeOH:NH₄OH, 90:9:1) to give 17.1 mg of pure product (67%). ¹³C NMR (151 MHz, DMSO-d₆) δ 168.53, 166.99, 158.72, 145.70, 141.77, 128.77, 128.05, 126.17, 125.97, 125.30, 123.31, 70.70, 57.97, 41.73, 39.52, 39.38, 39.24, 39.10, 38.58, 33.41, 25.89, 22.48, 22.17. ¹H NMR (400 MHz, DMSO-d₆) δ 8.87 (t, J=6.1 Hz, 1H), 8.06 (t, J=5.6 Hz, 1H), 8.01 (d, J=8.4 Hz, 1H), 7.90 (d, J=8.5 Hz, 1H), 7.67 (t, J=7.7 Hz, 1H), 7.52 (t, J=7.7 Hz, 1H), 3.97 (bd, 2H), 3.48-3.29 (m, 4H), 3.28 (s, 3H), 3.03 (t, J=6.8 Hz, 2H), 2.97-2.79 (m, 2H), 1.99-1.86 (m, 2H), 1.86-1.62 (m, 2H). LC-MS (ESI); m/z: [M+H]⁺ Calcd. for C₁₉H₂₄N₃O₃, 342.1817. Found 342.18.

### Methyl (1,2,3,4-tetrahydroacridine-9-carbonyl)glycylglycinate (compound 8)

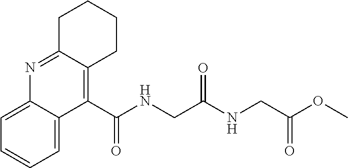 [SMILES: COC(=O)CNC(=O)CNC(=O)c1c2c(nc3ccccc13)CCCC2]

To a solution of 2-(1,2,3,4-tetrahydroacridine-9-carbonylamino)acetic acid (6) (27.4 mg, 0.0964 mmol) in DMF (2 mL) was added methyl 2-aminoacetate hydrochloride (14.5 mg, 0.116 mmol), Triethylamine (0.0672 mL, 0.482 mmol), and HATU (40.3 mg, 0.106 mmol) at room temperature. The reaction was stirred at the same temperature for 24 h. The reaction mixture was diluted with EtOAc (10 mL) and washed with water/brine (1:1, 3×10 mL), then the organic phase was separated, dried (Na₂SO₄), and evaporated under vacuum. The crude product was purified by PTLC (DCM:MeOH:NH₄OH, 90:9:1) to give 21.6 mg of pure product (63% yield). ¹H NMR (600 MHz, DMSO-d₆) δ 8.93 (t, J=5.7 Hz, 1H), 8.48 (t, J=5.4 Hz, 1H), 7.98 (d, J=8.2 Hz, 1H), 7.89 (d, J=8.4 Hz, 1H), 7.66 (t, J=7.6 Hz, 1H), 7.51 (t, J=7.5 Hz, 1H), 4.19-3.97 (m, 2H), 3.96 (d, J=5.6 Hz, 2H), 3.66 (s, 3H), 3.04 (t, J=6.1 Hz, 2H), 2.99-2.77 (m, 2H), 1.89 (q, J=6.5, 5.9 Hz, 2H), 1.81 (p, J=6.2 Hz, 2H). ¹³C NMR (151 MHz, DMSO-d₆) δ 170.78, 169.50, 167.40, 159.12, 146.08, 142.10, 129.17, 128.45, 126.59, 126.38, 125.68, 123.68, 52.19, 41.88, 41.03, 33.81, 26.28, 22.87, 22.55. LRMS (ESI); m/z: [M+H]⁺ Calcd. for C₁₉H₂₂N₃O₄, 356.1610. Found 356.26.

### N-(2-(Butylamino)-2-oxoethyl)-1,2,3,4-tetrahydroacridine-9-carboxamide (compound 9)

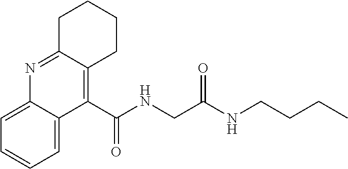 [SMILES: CCCCNC(=O)CNC(=O)c1c2c(nc3ccccc13)CCCC2]

To a solution of 2-(1,2,3,4-tetrahydroacridine-9-carbonylamino)acetic acid (6) (37.8 mg, 0.133 mmol) in DMF (2 mL) was added butan-1-amine (29.2 mg, 0.399 mmol), Triethylamine (0.0927 mL, 0.665 mmol), and HATU (55.6 mg, 0.146 mmol) at room temperature. The reaction was stirred at the same temperature for 24 hours. The reaction mixture was diluted with EtOAc (10 mL), washed with water/brine (1:1 3×10 mL), then the organic phase was separated, dried (Na₂SO₄, and evaporated under vacuum. The crude product was purified by PTLC (DCM:MeOH:NH₄OH, 90:9:1) to give 31.4 mg of pure product (67% yield). ¹H NMR (500 MHz, DMSO-d₆) δ 8.85 (t, J=5.2 Hz, 1H), 8.00 (d, J=8.3 Hz, 1H), 7.96 (t, J=5.5 Hz, 1H), 7.89 (d, J=8.4 Hz, 1H), 7.66 (t, J=7.5 Hz, 1H), 7.51 (t, J=7.5 Hz, 1H), 3.94 (bs, 2H), 3.13 (q, J=6.0 Hz, 2H), 3.04 (t, J=6.5 Hz, 2H), 2.96-2.81 (m, 2H), 2.01-1.85 (m, 2H), 1.87-1.70 (m, 2H), 1.43 (p, J=6.8 Hz, 2H), 1.38-1.25 (m, 2H), 0.89 (t, J=7.2 Hz, 3H). ¹³C NMR (126 MHz, DMSO-d₆) δ 168.18, 166.91, 158.67, 145.67, 141.75, 128.71, 128.02, 126.13, 125.90, 125.27, 123.28, 41.77, 38.30, 33.38, 31.26, 25.84, 22.44, 22.13, 19.56, 13.69. LC-MS (ESI); m/z: [M+H]⁺ Calcd. for C₂₀H₂₆N₃O₂, 340.2025. Found 340.41.

### 4-(4-Hydroxybenzylidene)-N-(2-((2-methoxyethyl)amino)-2-oxoethyl)-1,2,3,4-tetrahydroacridine-9-carboxamide (compound 10)

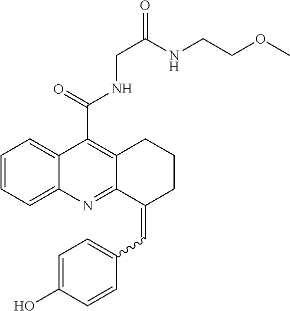 [SMILES: COCCNC(=O)CNC(=O)c1c2c(nc3ccccc13)C(=Cc1ccc(O)cc1)CCC2]

To a solution of N-[2-(2-methoxyethylamino)-2-oxo-ethyl]-2,3-dihydro-1H-cyclopenta[b]-quinoline-9-carboxamide (7) (16.0 mg, 0.0489 mmol) and 4-hydroxybenzaldehyde (7.16 mg, 0.0586 mmol) in DMF (1.00 mL) was added Chlorotrimethylsilane (26.5 mg, 0.244 mmol), and then stirred at 100° C. for 6 h in a sealed tube. By TLC approx. only 10% starting material was left (DCM:MeOH:NH₄OH, 90:9:1), at which point the reaction mixture was diluted with a mixture of EtOAc (10 mL) and aqueous saturated solution of NaHCO₃ (5 mL), the organic phase was washed with brine/water (4×5 mL), dried (Na₂SO₄), and evaporated under vacuum to give 22.2 mg of crude product. The crude product was purified by PTLC (DCM:MeOH:NH₄OH, 90:9:1) to give 8.8 mg of pure product (41% yield). ¹³C NMR (151 MHz, DMSO-d₆) δ 168.51, 166.99, 156.97, 153.79, 145.98, 141.68, 132.41, 131.42, 129.05, 128.92, 128.77, 127.88, 126.66, 126.14, 125.25, 123.47, 115.32, 70.68, 57.96, 41.75, 38.56, 27.94, 26.71, 22.11. ¹H NMR (600 MHz, DMSO-d₆) δ 9.69 (s, 1H), 8.92 (t, J=5.5 Hz, 1H), 8.15-8.05 (m, 2H), 7.98 (t, J=7.6 Hz, 2H), 7.75-7.63 (m, 1H), 7.57-7.47 (m, 1H), 7.40 (d, J=7.9 Hz, 2H), 6.84 (d, J=7.8 Hz, 2H), 4.00 (bd, J=29.1 Hz, 1H), 3.40 (t, J=5.3 Hz, 2H), 3.33-3.30 (m, 2H), 3.28 (s, 3H), 3.12-2.77 (m, 4H), 1.94-1.69 (m, 2H). LC-MS (ESI); m/z [M+1]⁺; Calcd. C₂₆H₂₈N₃O₄, 446.2079. Found 446.43.

### Methyl (4-(4-hydroxybenzylidene)-1,2,3,4-tetrahydroacridine-9-carbonyl)glycylglycinate (Compound 11)

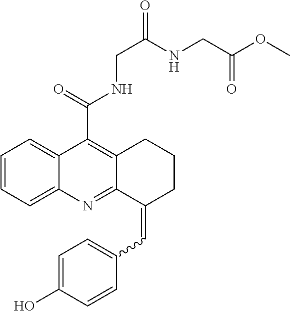 [SMILES: COC(=O)CNC(=O)CNC(=O)c1c2c(nc3ccccc13)C(=Cc1ccc(O)cc1)CCC2]

To a solution of methyl 2-[[2-(1,2,3,4-tetrahydroacridine-9-carbonylamino)acetyl]amino]acetate (8) (18.7 mg, 0.0526 mmol) and 4-hydroxybenzaldehyde (7.71 mg, 0.0631 mmol) in DMF (1.00 mL) was added Chlorotrimethylsilane (28.6 mg, 0.263 mmol). The system was then stirred at 100° C. for 28 h in a sealed tube, transferred to round bottom with 5 mL methanol. The methanol was removed under vacuum, and the residue was dried overnight under high vacuum to remove DMF. The crude product was re-dissolved in MeCN (2.00 mL), and CsF (16.0 mg, 0.105 mmol) was added and stirred for 6h at room temperature. The reaction mixture was diluted with DCM (20 mL) and aqueous saturated-solution of NaHCO₃ (10 mL), the organic layer was separated, and the aqueous layer was extracted again with DCM (20 mL). The organic extracts were combined, dried (Na₂SO₄), and evaporated under vacuum. The crude product was purified by PTLC (DCM:MeOH:NH₄OH, 90:9:1) to give 9.3 mg of pure product (39% yield). ¹H NMR (500 MHz, DMSO-d₆) δ 9.68 (s, 1H), 8.98 (t, J=5.8 Hz, 1H), 8.49 (t, J=5.7 Hz, 1H), 8.07 (s, 1H), 7.98 (d, J=8.4 Hz, 2H), 7.69 (t, J=7.7 Hz, 1H), 7.51 (t, J=7.6 Hz, 1H), 7.40 (d, J=8.4 Hz, 2H), 6.84 (d, J=8.4 Hz, 2H), 4.05 (bs, 2H), 3.97 (d, J=5.5 Hz, 2H), 3.67 (s, 3H), 3.07-2.71 (m, 4H), 1.88-1.74 (m, 2H). ¹³C NMR (151 MHz, DMSO-d₆) δ 170.36, 169.09, 167.02, 156.98, 153.79, 145.97, 141.61, 132.39, 131.42, 129.06, 128.94, 128.76, 127.88, 126.69, 126.16, 125.24, 123.45, 115.32, 51.78, 41.52, 40.62, 27.94, 26.71, 22.11. LC-MS (ESI); m/z [M+1]⁺; Calcd. C₂₆H₂₆N₃O₅, 460.1872. Found 460.44.

### N-(2-(Butylamino)-2-oxoethyl)-4-(4-hydroxybenzylidene)-1,2,3,4-tetrahydroacridine-9-carboxamide (compound 12)

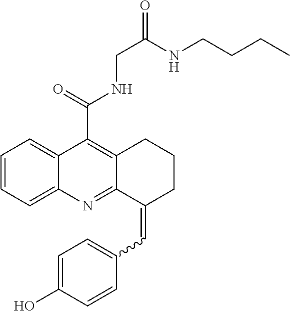 [SMILES: CCCCNC(=O)CNC(=O)c1c2c(nc3ccccc13)C(=Cc1ccc(O)cc1)CCC2]

To a solution of N-[2-(butylamino)-2-oxo-ethyl]-1,2,3,4-tetrahydroacridine-9-carboxamide (9) (26.9 mg, 0.0793 mmol) and 4-hydroxybenzaldehyde (11.6 mg, 0.0951 mmol) in DMF (1.00 mL) was added Chlorotrimethylsilane (43.0 mg, 0.396 mmol), and then the system stirred in a sealed tube for 34 h at 100° C. The reaction mixture was diluted with Ethyl Acetate (10 mL) and aqueous saturated-solution of NaHCO₃ (10 mL), and the organic layer was washed with water/brine (1:1, 10 mL). The organic extract was separated, dried (Na₂SO₄), and evaporated under vacuum. The crude product was purified by PTLC (DCM:MeOH:NH₄OH, 90:9:1) to give 12.1 mg of pure product (34% yield). LC-MS (ESI); m/z [M+1]⁺; Calcd. C₂₇H₃₀N₃O₃, 444.2287. Found 444.43.

### 1-(3-Ethylureido)-1-oxopropan-2-yl 2,3-dihydro-1H-cyclopenta[b]quinoline-9-carboxylate (Compound 13)

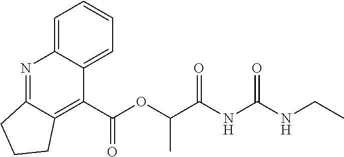 [SMILES: CCNC(=O)NC(=O)C(C)OC(=O)c1c2c(nc3ccccc13)CCC2]

To a suspension of 2,3-dihydro-1H-cyclopenta[b]quinoline-9-carboxylic acid (33.0 mg, 0.155 mmol) in DMF (2 mL) was added 2-chloro-N-(ethylcarbamoyl)propanamide (30.4 mg, 0.170 mmol) (36.9 mg, 0.206 mmol), and then N,N-Diisopropylethylamine (0.0270 mL, 0.155 mmol). The resulting solution was stirred at 4 h at 60° C. The reaction mixture was diluted with EtOAc (15 mL), washed with brine/water (1:1, 4×10 ml), dried (Na₂SO₄) and evaporated under vacuum. The crude product was purified by PTLC (DCM:MeOH:NH₄OH, 90:9:1) to give 25 mg of pure product as a white solid (41% yield). ¹H NMR (400 MHz, DMSO-d₆) δ 11.14 (bs, 1H), 8.68 (dd, J=8.5, 1.3 Hz, 1H), 8.52 (bs, 1H), 8.36 (dd, J=8.3, 1.5 Hz, 1H), 8.08 (ddd, J=8.4, 7.0, 1.4 Hz, 1H), 7.96 (ddd, J=8.3, 6.9, 1.4 Hz, 1H), 5.72 (d, J=7.4 Hz, 1H), 3.67-3.51 (m, 4H), 3.47 (t, J=7.7 Hz, 2H), 2.51 (ddt, J=12.3, 7.7, 3.3 Hz, 2H), 1.91 (d, J=6.9 Hz, 3H), 1.44 (t, J=7.2 Hz, 3H). ¹³C NMR (101 MHz, DMSO-d₆) δ 171.76, 168.08, 165.60, 152.61, 147.29, 135.62, 130.23, 128.89, 128.84, 126.79, 124.87, 122.67, 70.90, 34.02, 33.95, 30.56, 22.80, 17.06, 14.92. LC-MS (ESI); m/z [M+1]⁺; Calcd. C₁₉H₂₂N₃O₄, 356.1610. Found 356.16.

### 1-(3-ethylureido)-1-oxopropan-2-yl 3-(3,4-dimethoxybenzylidene)-2,3-dihydro-1H-cyclopenta[b]quinoline-9-carboxylate (compound 14)

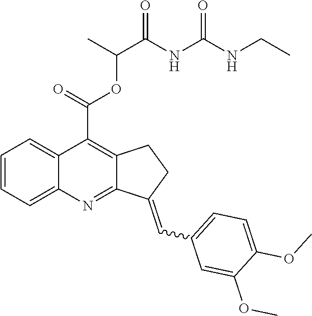 [SMILES: CCNC(=O)NC(=O)C(C)OC(=O)c1c2c(nc3ccccc13)C(=Cc1ccc(OC)c(OC)c1)CC2]

To a solution of [2-(ethylcarbamoylamino)-1-methyl-2-oxo-ethyl]-2,3-dihydro-1H-cyclopenta[b]quinoline-9-carboxylate (25.0 mg, 0.0703 mmol) and 2,3-dimethoxybenzaldehyde (14.0 mg, 0.0844 mmol) in DMF (1.00 mL) was added Chlorotrimethylsilane (38.2 mg, 0.352 mmol), and the system was stirred at 100° C. for 2 h in a sealed tube. When no more starting materials was observed by TLC (Hex:EtOAc, 1:1), the reaction mixture was diluted with a mixture of EtOAc (10 mL) and aqueous-saturated solution of NaHCO₃ (5 mL). The organic phase was separated, washed with brine/water (4×5 mL), dried (Na₂SO₄), and evaporated under vacuum. The crude product was purified by PTLC (DCM:MeOH:NH₄OH, 90:9:1) to give 25 mg of pure product (70% yield). ¹H NMR (400 MHz, DMSO-d₆) δ 10.80 (bs, 1H), 8.38 (d, J=8.4 Hz, 1H), 8.17 (bs, 1H), 8.12 (d, J=8.4 Hz, 1H), 7.99 (s, 1H), 7.77 (t, J=7.6 Hz, 1H), 7.63 (t, J=7.6 Hz, 1H), 7.28 (d, J=7.8 Hz, 1H), 7.16 (t, J=8.0 Hz, 1H), 7.07 (d, J=8.1 Hz, 1H), 5.40 (d, J=6.9 Hz, OH), 3.84 (s, 3H), 3.80 (s, 3H), 3.51-3.35 (m, 2H), 3.29-2.89 (m, 4H), 1.57 (d, J=6.9 Hz, 3H), 1.09 (t, J=7.2 Hz, 3H). ¹³C NMR (101 MHz, DMSO-d₆) δ 171.76, 165.42, 161.98, 152.62, 152.60, 148.03, 147.51, 140.90, 137.37, 131.10, 130.44, 129.43, 129.43, 127.25, 124.95, 123.98, 123.66, 120.51, 118.83, 112.80, 70.99, 60.72, 55.73, 34.04, 28.26, 28.01, 17.07, 14.92. LC-MS (ESI); m/z [M+1]⁺; Calcd. C₂₈H₃₀N₃O₆, 504.2134. Found 504.21.

### Example 2: Preparation of Compounds Ac-RVSF, Oct-RVSF, and H₂N-RVSF (Protein Phosphatase Ligands)

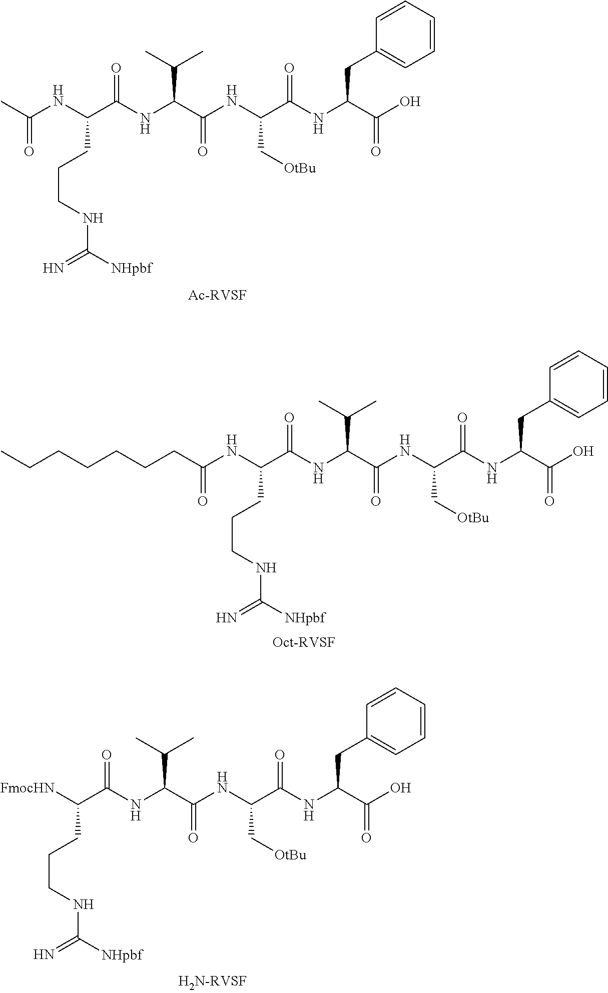

The title compounds were synthesized via solid phase peptide synthesis and resin cleavage, according to the general synthetic procedure set forth in the scheme below.

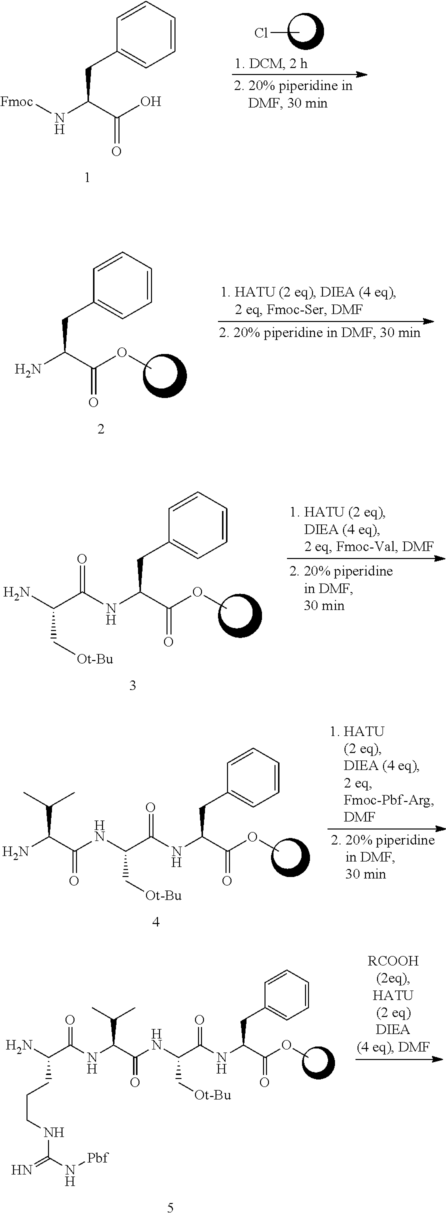

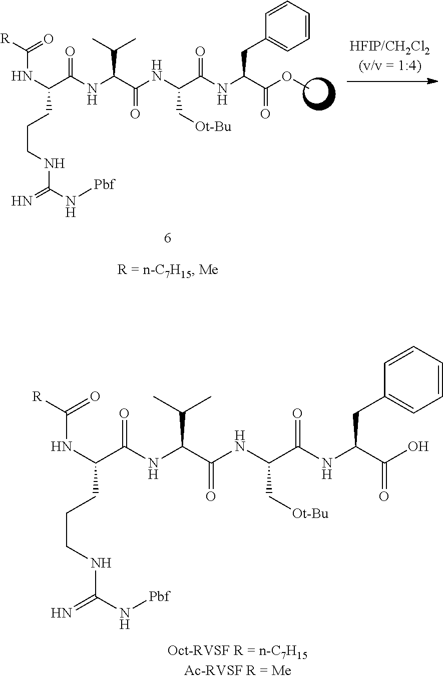

### Part I-General Procedure for Attaching First Amino Acid to Resin

A mixture of 1 (10 g, 25.8 mmol, 1.0 equiv) and resin (26 g) in DCM (100 mL) was agitated with N₂ bubbling and then DIEA (16.7 g, 130 mmol, 22.5 mL, 5 equiv) was added to the mixture. The mixture was agitated with N₂ bubbling at 25° C. for 2 h. The solvent was filtered and the resin was washed with DMF (3×30 mL).

### Part II-General Procedure for the Amide Formation and Cleavage of Fmoc on Resin

A mixture of resin (25.7 mmol, 1.0 equiv), Fmoc protected amino acid (64.2 mmol, 2.5 equiv), HATU (24.4 g, 64.2 mmol, 2.5 equiv) and DIPEA (16.6 g, 128 mmol, 22.4 mL, 5 equiv) in DMF (50 mL) was agitated with N₂ bubbling at 25° C. for 1 h. Ninhydrin coloration showed the reaction was complete. The mixture was filtered and the resin was washed with DMF (3×30 mL). Then the resin in 20% (V/V) piperidine/DMF (100 mL) was agitated with bubbling N₂ for 30 min. Then the mixture was filtered to get the crude product which was used for next step directly.
Part III-General Procedure for Cleavage of Peptide from Resin
The peptide attached resin in a HFIP solution (10 mL, 20% v/v in DCM) was agitated with bubbling N₂ at 20° C. for 1.5 h. The mixture was filtrated and the filtrate was concentrated to give the crude peptide, which was further purified by prep-HPLC (column: Phenomenex luna C18 (250*70 mm, 10 um); mobile phase: [water (0.225% FA)-ACN]; B %: 55%-90%, 22 min).

### Physical Characterization Data for Compounds Oct-RVSF, Ac-RVSF, and H₂N-RVSF:

Ac-RVSF: (2S)-2-[[(2S)-2-[[(2S)-2-[[(2S)-2-acetamido-5-[[N-[(2,2,4,5,7-pentamethyl-3H-benzofuran-6-yl)sulfonyl]carbamimidoyl]amino]pentanoyl]amino]-3-methyl-butanoyl]amino]-3-tert-butoxy-propanoyl]amino]-3-phenyl-propanoic acid. LC-MS: MS (ES⁺): RT=0.873 min, m/z=858.0 [M+H+].
Oct-RVSF: (2S,5S,8S,11S)-2-benzyl-5-(tert-butoxymethyl)-8-isopropyl-4,7,10,13-tetraoxo-11-(3-(3-((2,2,4,6,7-pentamethyl-2,3-dihydrobenzofuran-5-yl)sulfonyl)guanidino)propyl)-3,6,9,12-tetraazaicosan-1-oic acid. ¹HNMR (400 MHz, CD₃OD): δ 7.96-8.08 (m, 1H), 7.12-7.34 (m, 5H), 4.70 (dd, 1H, J=7.2, 5.50 Hz), 4.35-4.51 (m, 2H), 4.19-4.29 (m, 1H), 3.51-3.66 (m, 2H), 3.11-3.20 (m, 2H), 2.96-3.05 (m, 3H), 2.59 (s, 3H), 2.53 (s, 3H), 2.24 (t, 2H, J=7.5 Hz), 2.03-2.16 (m, 4H), 1.73-1.87 (m, 1H), 1.50-1.69 (m, 5H), 1.47 (s, 6H), 1.25-1.40 (m, 8H), 1.15 (s, 9H), 0.83-0.99 (m, 9H). LC-MS: MS (ES⁺): RT=1.08 min, m/z=942.6 [M+H⁺].
H₂N-RVSF: (5S,8S,11S,14S)-14-benzyl-11-(tert-butoxymethyl)-1-(9H-fluoren-9-yl)-8-isopropyl-3,6,9,12-tetraoxo-5-(3-(3-((2,2,4,6,7-pentamethyl-2,3-dihydrobenzofuran-5-yl)sulfonyl)guanidino)propyl)-2-oxa-4,7,10,13-tetraazapentadecan-15-oic acid. ¹HNMR (400 MHz, CD₃OD): δ 7.99 (s, 1H), 7.80 (m, 2H), 7.66 (m, 2H), 7.35-7.45 (m, 2H), 7.29-7.34 (m, 2H), 7.18-7.26 (m, 5H), 4.61-4.75 (m, 3H), 4.35-4.50 (m, 3H), 4.20-4.28 (m, 2H), 4.08-4.19 (m, 1H), 3.46-3.66 (m, 2H), 3.16 (m, 3H), 2.99-3.07 (m, 5H), 2.97 (s, 2H), 2.87 (s, 4H), 2.60 (s, 3H), 2.53 (s, 3H), 2.02-2.15 (m, 4H), 1.78 (m, 1H), 1.45-1.69 (m, 4H), 1.43 (s, 6H), 1.12 (s, 9H), 0.92 (m, 6H). LC-MS: MS (ES⁺): RT=1.992 min, m/z=297.1 [M+H⁺].

### Example 3: Preparation of Compound SMAP-Direct (Protein Phosphatase Ligand)

The title compound was prepared according to the scheme and procedures below. Compound 3a is described in WO2015/138500.

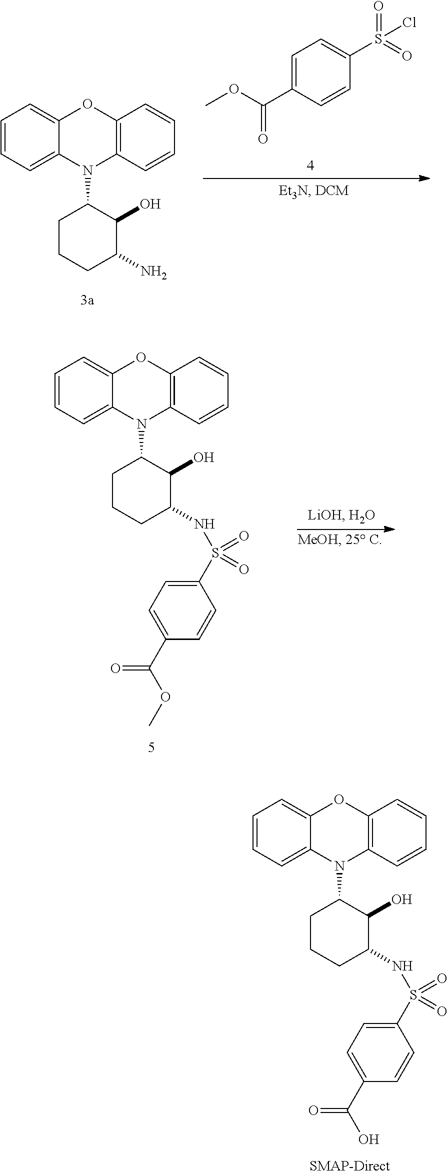

### Part I-Preparation of Compound 5

To a mixture of compound 3a (1.0 g, 3.4 mmol, 1 equiv), Et₃N (512 mg, 5.1 mmol, 1.5 equiv) in DCM (10 mL) was added compound 4 (870 mg, 3 mmol, 1.1 equiv) at 0° C. The mixture was stirred at 25° C. for 3 h under N₂ protection. The mixture was concentrated and purified by column chromatography (SiO₂, Petroleum ether/Ethyl acetate=5/1 to 3/1) to give compound 5 (1.3 g, 77% yield). ¹H NMR (400 MHz, DMSO-d₆) δ 8.09 (d, 2H, J=8.8 Hz), 7.96 (d, 2H, J=8.4 Hz), 7.77 (d, 1H, J=7.2 Hz), 6.82-6.93 (m, 4H), 6.67-6.78 (m, 4H), 4.89 (d, 1H, J=6.0 Hz), 3.79-3.93 (m, 4H), 3.41-3.55 (m, 1H), 3.04 (m, 1H), 1.73-1.95 (m, 2H), 1.49-1.69 (m, 2H), 1.19-1.41 (m, 2H).

### Part II-Preparation of Compound SMAP-Direct

A mixture of compound 5 (200 mg, 404 μmol, 1.0 equiv), LiOH (19 mg, 808 μmol, 2.0 equiv) in MeOH (1 mL) and H₂O (1 mL) was stirred at 25° C. for 2 h. The organic solvent was evaporated, and the residue was adjust to pH=5-7 by addition of 12 M HCl. The suspension was filtered and dried to give SMAP-Direct (180 mg, 92% yield) as a white solid. ¹H NMR (400 MHz, DMSO-d₆) δ 8.06 (d, 2H, J=8.4 Hz), 7.94 (d, 2H, J=8.4 Hz), 7.73 (d, 1H, J=7.6 Hz), 6.80-6.94 (m, 4H), 6.66-6.79 (m, 4H), 3.84 (m, 1H), 3.38-3.39 (m, 1H), 3.03 (m, 1H), 1.77-1.91 (m, 2H), 1.52-1.72 (m, 2H), 1.28 (m, 2H).

### Example 4: Preparation of Compound SMAP-4DiF (Protein Phosphatase Ligand)

The title compound was prepared according to the scheme and procedures below. Compound 3a is described in WO2015/138500.

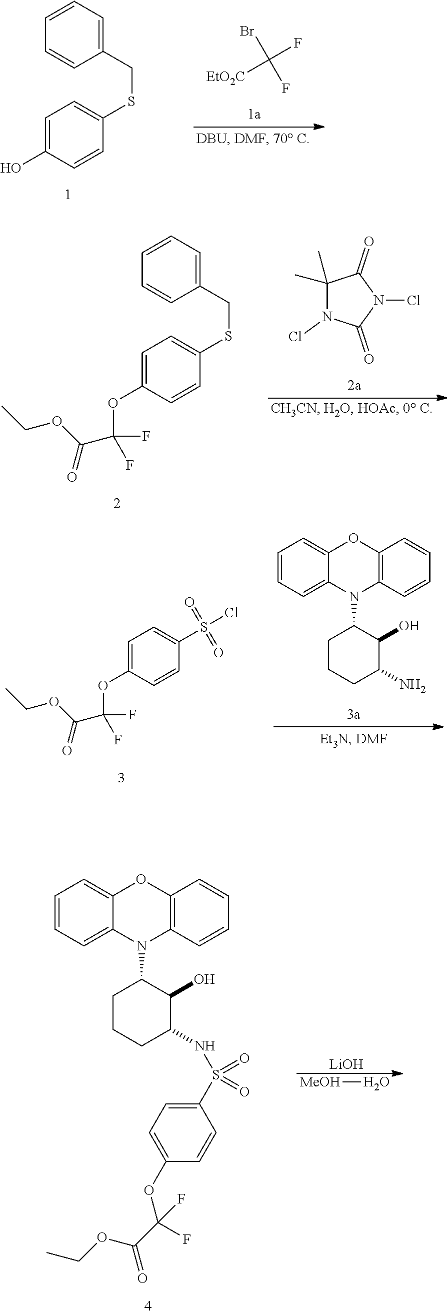

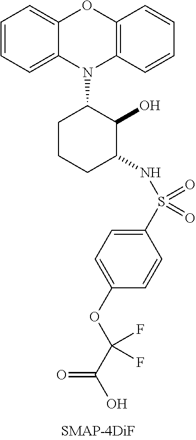 [SMILES: O=C(O)C(F)(F)Oc1ccc(S(=O)(=O)N[C@@H]2CCC[C@H](N3c4ccccc4Oc4ccccc43)[C@H]2O)cc1]

### Part I-Preparation of Compound 2

To a mixture of 4-benzylsulfanylphenol (6.5 g, 30 mmol, 19 mL, 1.0 equiv) and DBU (6.9 g, 45 mmol, 7.0 mL, 1.5 equiv) in DMF (10 mL) was added ethyl 2-bromo-2,2-difluoro-acetate (15 g, 75 mmol, 9.6 mL, 2.5 equiv) at 70° C. The mixture was stirred at 70° C. for 12 h. The mixture was poured into water (300 mL) and extracted with ethyl acetate (3×300 mL). The combined organic layers were concentrated and purified by column (Petroleum ether:Ethyl acetate=3:1) to give compound 2 (9.5 g, 93% yield) as a colorless oil. 1H NMR (400 MHz, CD₃OD): δ 7.31-7.26 (m, 7H), 7.13-7.11 (m, 2H), 4.40 (q, 2H, J=3.2 Hz), 4.11 (s, 2H), 1.37 (t, 3H, J=3.2 Hz).

### Part II-Preparation of Compound 3

To a mixture of ethyl 2-(4-(benzylthio)phenoxy)-2,2-difluoroacetate 2 (3.0 g, 8.9 mmol, 1.0 equiv) in CH₃CN (60 mL), HOAc (3 mL) and H₂O (2 mL) was added 2a (3.5 g, 18 mmol, 2.0 equiv) at 0° C. The mixture was stirred at 0° C. for 2 h. The mixture was quenched with aq. NaHCO₃ (200 mL) and extracted with ethyl acetate (3×100 mL). The combined organic layers were concentrated and purified by column (Petroleum ether:Ethyl acetate=3:1) to give compound 3 (1.9 g, 69% yield) as a brown oil. ¹H NMR (400 MHz, CD₃OD): δ 8.11-8.09 (m, 2H), 7.48-7.46 (m, 2H), 4.44 (q, 2H, J=7.2 Hz), 1.43-1.39 (t, 3H, J=7.2 Hz).

### Part III-Preparation of Compound 4

To a mixture of 3a (1.5 g, 5.1 mmol, 1.0 equiv) and DIPEA (1.9 g, 15 mmol, 2.6 mL, 3.0 equiv) in DMF (10 mL) was added compound 3 (1.8 g, 5.7 mmol, 1.2 equiv) at 20° C., and then the mixture was stirred at 20° C. for 12 h. The mixture was concentrated and purified by column (Petroleum ether:Ethyl acetate=3:1˜1:1) to give compound 4 (2.3 g, 79% yield) as a pink solid. ¹H NMR (400 MHz, CD₃OD): δ 7.97-7.95 (m, 2H), 7.37-7.35 (m, 2H), 6.94-6.72 (m, 8H), 4.35 (q, 2H, J=7.2 Hz), 3.85-3.80 (m, 1H), 3.40-3.37 (m, 1H), 3.08-3.07 (m, 1H), 1.92-1.62 (m, 4H), 1.31-1.23 (m, 5H); LC-MS: MS (ES⁺): RT=2.729 min, m/z=575.1 [M+H]⁺.

### Part IV-Preparation of SMAP-4DiF

To a mixture of 4 (300 mg, 535 μmol, 1 equiv) in MeOH (5 mL), H₂O (3 mL) was added LiOH (19 mg, 802 μmol, 1.5 equiv). Then the mixture was stirred for 3 h at 25° C. under N₂. The mixture was concentrated and purified by prep-HPLC (column: Waters X-bridge 150*25 mm*5 um; mobile phase: [water (0.05% ammonia hydroxide v/v)-ACN]; B %: 11%-41%, 10 min) to give SMAP-4DiF (260 mg, 88% yield) as a white solid. ¹H NMR (400 MHz, DMSO-d₆): δ 7.81-7.79 (m, 2H), 7.47-7.46 (m, 1H), 7.24-7.22 (m, 2H), 6.91-6.86 (m, 4H), 6.75-6.72 (m, 4H), 4.97-4.97 (m, 1H), 3.89-3.84 (m, 1H), 3.53-3.50 (m, 1H), 3.05-2.98 (m, 1H), 1.91-1.82 (m, 2H), 1.66-1.54 (m, 2H), 1.33-1.22 (m, 2H); LC-MS: MS (ES⁺): RT=1.949 min, m/z=574.1 [M+H]⁺.

### Example 5: Preparation of Compound SMAP-3DiF (Protein Phosphatase Ligand)

The title compound was prepared according to the scheme and procedures below. Compound 3a is described in WO2015/138500.

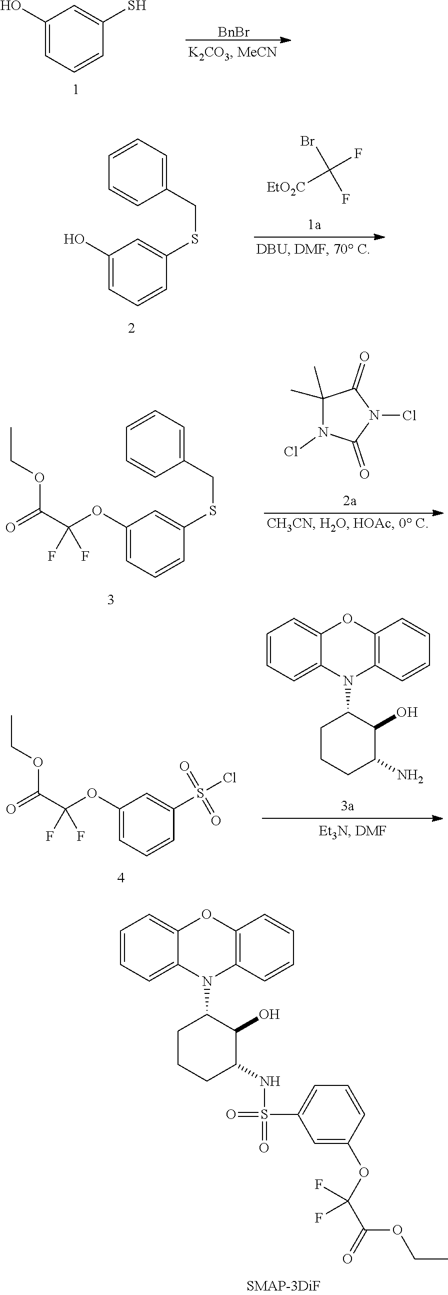

### Part I-Procedure for the Preparation of Compound 2

To a mixture of 1 (500 mg, 3.96 mmol, 1 equiv) and BnBr (711 mg, 4.16 mmol, 1.1 equiv) in MeCN (10 mL) was added K₂CO₃ (1.64 g, 11.9 mmol, 3.0 equiv). The mixture was stirred at 25° C. for 72 h. The mixture was filtered, and the filtrate was concentrated to afford 2. ¹H NMR (DMSO-d₆, 400 MHz) δ 9.52 (s, 1H), 7.37-7.22 (m, 5H), 7.17-7.13 (m, 1H), 6.76-6.74 (m, 2H), 6.71-6.65 (m, 1H), 4.19 (s, 2H); LC-MS: MS (ES⁺): m/z=217.1 [M+H]⁺.

### Part II-Procedure for the Preparation of Compound 3

To a solution of 2 (19.0 g, 87.8 mmol, 1.0 equiv) in DMF (100 mL) were added DBU (20.1 g, 132 mmol, 19.9 mL, 1.5 equiv) and 1a (44.6 g, 220 mmol, 2.5 equiv). The mixture was stirred at 70° C. for 16 h. The mixture was quenched with H₂O (200 mL) and extracted with ethyl acetate (3×500 mL). The combined organic layers were concentrated. The resulting residue was purified by silica gel column (Petroleum ether:Ethyl acetate=3:1) to afford 3 (23 g, 77% yield). ¹HNMR (CD₃OD, 400 MHz): δ 7.17-7.36 (m, 7H), 7.12 (s, 1H), 6.94-7.06 (m, 1H), 4.32-4.39 (m, 2H), 4.17 (s, 2H), 1.27-1.33 (m, 3H).

### Part III-Procedure for the Preparation of Compound 4

To a solution of 3 (3.0 g, 8.9 mmol, 1.0 equiv) in MeCN (120 mL) and HOAc (6 mL) and H₂O (4 mL) was added 2a (3.5 g, 18 mmol, 2.0 equiv). The mixture was stirred at 0° C. for 2 h and quenched with aq. NaHCO₃ (200 mL). The mixture was extracted with ethyl acetate (3×100 mL) and the combined organic layers were concentrated. The resulting residue was purified by silica gel column (Petroleum ether:Ethyl acetate=3:1) to afford 4. ¹H NMR (CDCl₃, 400 MHz) δ 7.96-7.99 (m, 1H), 7.92-7.93 (m, 1H), 7.64-7.70 (m, 1H), 7.32-7.38 (m, 1H), 4.45 (q, 2H, J=7.2 Hz), 1.42 (t, 3H, J=7.2 Hz).

### Part IV-Procedure for the Preparation of Compound SMAP-3DiF

To a solution of 4 (1 g, 3.4 mmol, 1.0 equiv) in DCM (20 mL) was added DIPEA (872 mg, 6.8 mmol, 1.2 mL, 2.0 equiv) and 3a (1.2 g, 3.7 mmol, 1.1 equiv). The mixture was stirred at 0° C. for 16 h and quenched with water (20 mL). The mixture was extracted with ethyl acetate (3×50 mL) and the combined organic layers were concentrated. The resulting residue was purified by silica gel column (Petroleum ether:Ethyl acetate=3:1) to afford SMAP-3DiF. ¹H NMR (DMSO-d₆, 400 MHz) δ 7.21-7.93 (m, 5H), 6.71-6.89 (m, 7H), 4.02 (q, 2H, J=7.1 Hz), 3.82-3.86 (m, 1H), 3.46-3.51 (m, 1H), 3.01-3.05 (m, 1H), 1.98-1.53 (m, 6H), 1.28-1.22 (m, 3H); LC-MS: MS (ES⁺): m/z=575.2 [M+H]⁺.

### Example 6: Preparation of Compound JNS 1-40 (Protein Phosphatase Ligand)

The title compound was prepared according to the scheme and procedures below.

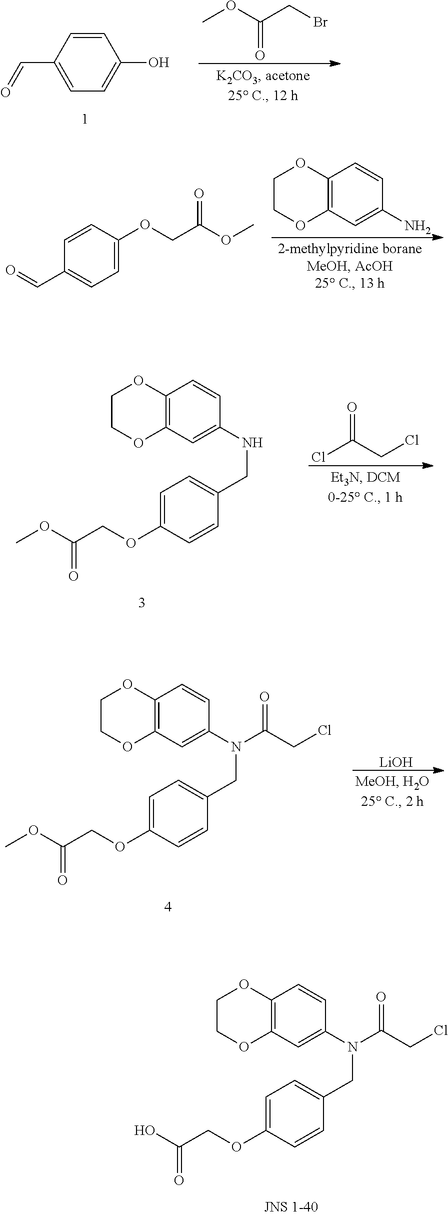

### Part I-Preparation of Compound 2 (Methyl 2-(4-Formylphenoxy)Acetate)

To a solution of 4-hydroxybenzaldehyde (5.00 g, 40.9 mmol, 1.0 equiv) in acetone (100 mL) was added K₂CO₃ (8.49 g, 61.4 mmol, 1.5 equiv) and methyl 2-bromoacetate (7.52 g, 49.1 mmol, 1.2 equiv). The mixture was stirred at 25° C. for 12 h. The reaction mixture was filtered and concentrated. The residue was diluted with water (150 mL) and extracted with ethyl acetate (3×200 mL). The combined organic layers were washed with brine (200 mL), dried over sodium sulfate, filtered and concentrated under reduced pressure to give a residue. Methyl 2-(4-formylphenoxy)acetate, 2 (10.00 g, crude) was used for next step reaction without further purification. ¹H NMR (400 MHz, CDCl₃) δ 9.89 (s, 1H), 7.76-7.90 (m, 2H), 6.92-7.03 (m, 2H), 4.72 (s, 2H), 3.81 (s, 3H); LC-MS: MS (ES⁺): m/z=195.1 [M+H⁺].

### Part II-Preparation of Compound 3 (Methyl 2-(4-(((2,3-dihydrobenzo[b][1,4]dioxin-6-yl)amino)methyl)phenoxy)acetate)

To a solution of 2 (0.10 g, 0.51 mmol, 1.0 equiv) in MeOH (2 mL) and AcOH (0.2 mL) was added 2,3-dihydro-1,4-benzodioxin-6-amine (0.08 g, 0.5 mmol, 1.0 equiv). The mixture was stirred at 25° C. for 1 h. Then 2-methylpyridine borane (0.11 g, 1.0 mmol, 2.0 equiv) was added, and the mixture was stirred for 12 h. The reaction mixture was filtered and concentrated in vacuo. The resulting residue was diluted with water (15 mL) and extracted with DCM (3×20 mL). The combined organic layers were washed with brine (20 mL), dried over sodium sulfate, filtered and concentrated under reduced pressure to give a residue. The residue was purified by prep-TLC (Petroleum ether:Ethyl acetate=1/1). Methyl 2-(4-(((2,3-dihydrobenzo[b][1,4]dioxin-6-yl)amino)methyl)phenoxy)acetate, 3 (0.14 g, 0.40 mmol, 82% yield), was obtained as a brown oil. ¹H NMR (400 MHz, CDCl₃) δ 7.31-7.26 (m, 2H), 6.85-6.90 (m, 2H), 6.72-6.67 (m, 1H), 6.21-6.16 (m, 2H), 4.64 (s, 2H), 4.24-4.22 (m, 2H), 4.20-4.18 (m, 4H), 3.82 (s, 3H); LC-MS: MS (ES⁺): m/z=330.1 [M+H⁺].

### Part III-Preparation of Compound 4 (Methyl 2-(4-((2-chloro-N-(2,3-dihydrobenzo[b][1,4]dioxin-6-yl)acetamido)methyl)phenoxy)acetate)

To a solution of 3 (0.14 g, 0.43 mmol, 1.0 equiv) in DCM (3 mL) was added triethyl amine (90 mg, 0.85 mmol, 2.0 equiv) and 2-chloroacetyl chloride (60 mg, 0.51 mmol, 1.2 equiv) at 0° C. The mixture was stirred at 25° C. for 1 h. The mixture was concentrated to give a residue. The residue was purified by prep-HPLC on a Phenomenex Synergi 150×25×10 um C18 column with HCl-modified water/acetonitrile mobile phase. Methyl 2-(4-((2-chloro-N-(2,3-dihydrobenzo[b][1,4]dioxin-6-yl)acetamido)methyl)phenoxy)acetate, 4 (0.01 g, 0.03 mmol, 8% yield, 99.2% purity) was obtained as an off-white gum. ¹H NMR (400 MHz, DMSO-d₆) δ 7.10 (d, 2H, J=8.7 Hz), 6.87-6.81 (m, 3H), 6.77 (d, 1H, J=2.3 Hz), 6.63 (dd, 1H, J=2.4, 8.6 Hz), 4.76 (s, 2H), 4.73 (s, 2H), 4.22 (s, 4H), 4.06 (s, 2H), 3.69 (s, 3H); LC-MS (Method 01): MS (ES⁺): RT=2.791 min, m/z=406.2, 408.2 [M+H⁺].

### Part IV-Preparation of JNS 1-40 (2-(4-((2-chloro-N-(2,3-dihydrobenzo[b][1,4]dioxin-6-yl)acetamido)methyl)phenoxy)acetic acid)

To a solution of 4 (0.55 g, 1.3 mmol, 1.0 equiv) in H₂O (2 mL) and MeOH (5 mL) was added LiOH•H₂O (60 mg, 1.4 mmol, 1.0 equiv). The mixture was stirred at 25° C. for 2 h. The reaction mixture was adjusted to pH=5 with HCl (1 M) and concentrated to give a residue. The residue was purified by prep-HPLC on a Phenomenex luna 150×40 mm×15 um C18 column with HCl modified water/acetonitrile mobile phase. 2-(4-((2-chloro-N-(2,3-dihydrobenzo[b][1,4]dioxin-6-yl)acetamido)methyl)phenoxy)acetic acid, JNS 1-40 (0.40 g, 1.0 mmol, 74% yield, 99.16% purity) was obtained as a white solid. ¹H NMR (400 MHz, DMSO-d₆) δ 7.09 (d, 2H, J=8.5 Hz), 6.85-6.80 (m, 3H), 6.77 (d, 1H, J=2.3 Hz), 6.63 (dd, 1H, J=2.5, 8.5 Hz), 4.73 (s, 2H), 4.60 (s, 2H), 4.22 (s, 4H), 4.05 (s, 2H); LC-MS (Method 01): MS (ES⁺): RT=2.532 min, m/z=392.0 394.0 [M+H⁺].

### Example 7: Preparation of Compounds (0-4)PEG-Chloroalkane (Linker-Target Protein Ligand)

The title compounds were prepared according to the general synthetic procedures below, as described and depicted in the scheme. Compound 1 in the following scheme is described in Singh, V.; Wang, S. L.; Kool, E. T. J. Am. Chem. Soc. 2013, 135, 6184-6191. Compound 1 in the following scheme is described in

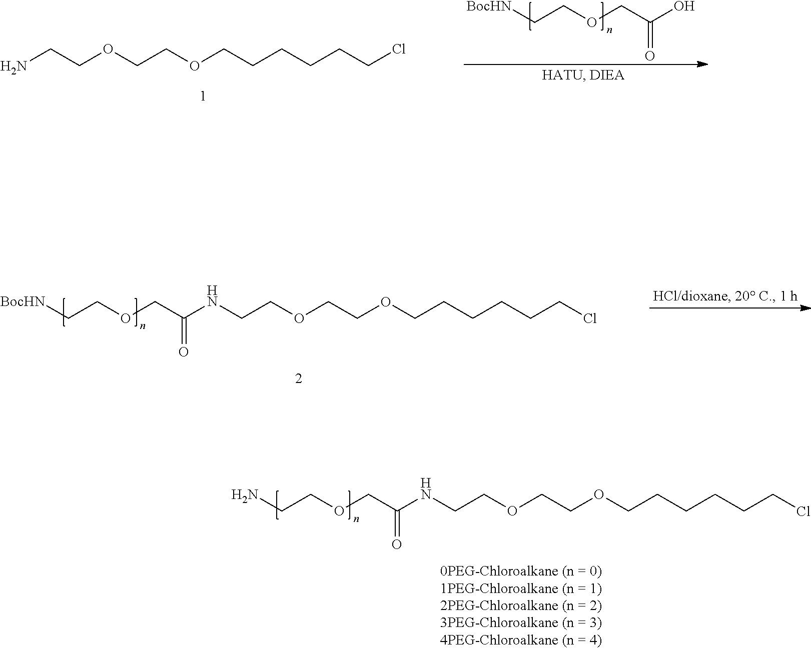

### Part I-General Procedure for Compounds 2a-e

To a solution of Compound 1 (1.15 mmol, 1.0 equiv, HCl salt), BocNH-PEG-COOH (1.27 mmol, 1.1 equiv) and DIEA (3.46 mmol, 0.6 mL, 3.0 equiv) in CH₂Cl₂ (3 mL) was added HATU (1.73 mmol, 1.5 equiv). The mixture was then stirred at 20° C. for 1 h. The reaction mixture was quenched by addition of H₂O (0.2 mL) at 0° C. and EtOAc (20 mL) was added. The organic phase was washed with 1 N HCl (3×10 mL), brine (2×10 mL), dried over Na₂SO₄, filtered and concentrated under reduced pressure. The resulting residue was purified by silica gel chromatography to give compound 2 (crude) as a yellow oil.

### Physical Characterization Data for Compounds 2a-e:

Compound 2a (tert-butyl (2-((2-(2-((6-chlorohexyl)oxy)ethoxy)ethyl)amino)-2-oxoethyl)carbamate) LC-MS: MS (ES⁺): RT=0.722 min, m/z=380.9 [M+H⁺].
Compound 2b (tert-butyl (18-chloro-5-oxo-3,9,12-trioxa-6-azaoctadecyl)carbamate)¹H NMR (400 MHz, CDCl₃): δ 7.00 (s, 1H), 5.10 (s, 1H), 3.98 (s, 2H), 3.4-3.7 (m, 16H), 3.35 (m, 2H), 2.81 (s, 2H), 1.80-1.77 (m, 2H), 1.6-1.7 (m, 4H), 1.46 (s, 9H), 1.3-1.4 (m, 2H); LC-MS: MS (ES⁺): RT=0.750 min, m/z=425.0 [M+H⁺].
Compound 2c (tert-butyl (21-chloro-8-oxo-3,6,12,15-tetraoxa-9-azahenicosyl)carbamate) LC-MS: MS (ES⁺): RT=0.746 min, m/z=469.0 [M+H⁺].
Compound 2d (tert-butyl (24-chloro-11-oxo-3,6,9,15,18-pentaoxa-12-azatetracosyl)carbamate) LC-MS: MS (ES⁺): RT=0.750 min, m/z=513.1 [M+H⁺].
Compound 2e (tert-butyl (27-chloro-14-oxo-3,6,9,12,18,21-hexaoxa-15-azaheptacosyl)carbamate) LC-MS: MS (ES⁺): RT=0.753 min, m/z=557.1 [M+H⁺].
Part II-General procedure for compounds (0-4)PEG-Chloroalkane
To a solution of compound 2 (551 μmol, 1.0 equiv) in dioxane (3 mL) was added HCl/dioxane (4 M, 5 mL). The mixture was stirred at 20° C. for 1 h. The mixture was concentrated to afford compound (0-4)PEG-Chloroalkane as a yellow solid (HCl salt), which was used for the next step directly.

### Physical Characterization Data for Compounds (0-4)PEG-Chloroalkane:

Compound 0PEG-Chloroalkane (2-amino-N-(2-(2-((6-chlorohexyl)oxy)ethoxy)ethyl) acetamide)¹H NMR (400 MHz, DMSO-d₆): δ 8.45 (s, 1H), 8.05 (s, 3H), 3.50-3.59 (m, 12H), 3.28 (m, 2H), 1.61-1.79 (m, 2H), 1.43-1.52 (m, 2H), 1.24-1.42 (m, 4H).
Compound 1PEG-Chloroalkane (2-(2-aminoethoxy)-N-(2-(2-((6-chlorohexyl)oxy)ethoxy) ethyl)acetamide)¹H NMR (400 MHz, DMSO-d₆): δ 8.11 (s, 4H), 3.91 (s, 2H), 3.59-3.69 (m, 6H), 3.42-3.50 (m, 6H), 3.23-3.31 (m, 2H), 3.01-3.02 (m, 2H), 1.65-1.79 (m, 2H), 1.23-1.58 (m, 6H).
Compound 2PEG-Chloroalkane (2-(2-(2-aminoethoxy)ethoxy)-N-(2-(2-((6-chlorohexyl)oxy) ethoxy)ethyl)acetamide)¹H NMR (400 MHz, DMSO-d₆): δ 7.96-7.71 (m, 4H), 3.90 (s, 2H), 3.60-3.63 (m, 6H), 3.57 (s, 2H), 3.42-3.51 (m, 6H), 3.37-3.38 (m, 2H), 3.24-3.29 (m, 2H), 2.95-3.02 (m, 2H), 1.67-1.74 (m, 2H), 1.28-1.52 (m, 6H).
Compound 3PEG-Chloroalkane (2-(2-(2-(2-aminoethoxy)ethoxy)ethoxy)-N-(2-(2-((6-chlorohexyl)oxy)ethoxy)ethyl)acetamide)¹H NMR (400 MHz, DMSO-d6): δ 7.91-7.69 (m, 4H), 3.90 (s, 2H), 3.58-3.65 (m, 10H), 3.37-3.53 (m, 11H), 3.24-3.30 (m, 2H), 2.91-3.05 (m, 2H), 1.63-1.80 (m, 2H), 1.43-1.54 (m, 2H), 1.25-1.43 (m, 4H).
Compound 4PEG-Chloroalkane (14-amino-N-(2-(2-((6-chlorohexyl)oxy)ethoxy)ethyl)-3,6,9,12-tetraoxatetradecan-1-amide)¹H NMR (400 MHz, DMSO-d6): δ 7.92-7.70 (m, 4H), 3.89 (s, 2H), 3.56-3.62 (m, 14H), 3.40-3.53 (m, 10H), 3.24-3.30 (m, 2H), 2.91-3.03 (m, 2H), 1.65-1.76 (m, 2H), 1.25-1.55 (m, 6H).

### Example 8: Preparation of Compounds (0-4)PEG-AKTallo (Linker-Target Protein Ligand)

The title compounds were prepared according to the general synthetic procedures below, as described and depicted in the scheme. Preparation of Ligand 2 is described in Angew. Chem. Int. Ed. 2015, 54, 10313-10316.

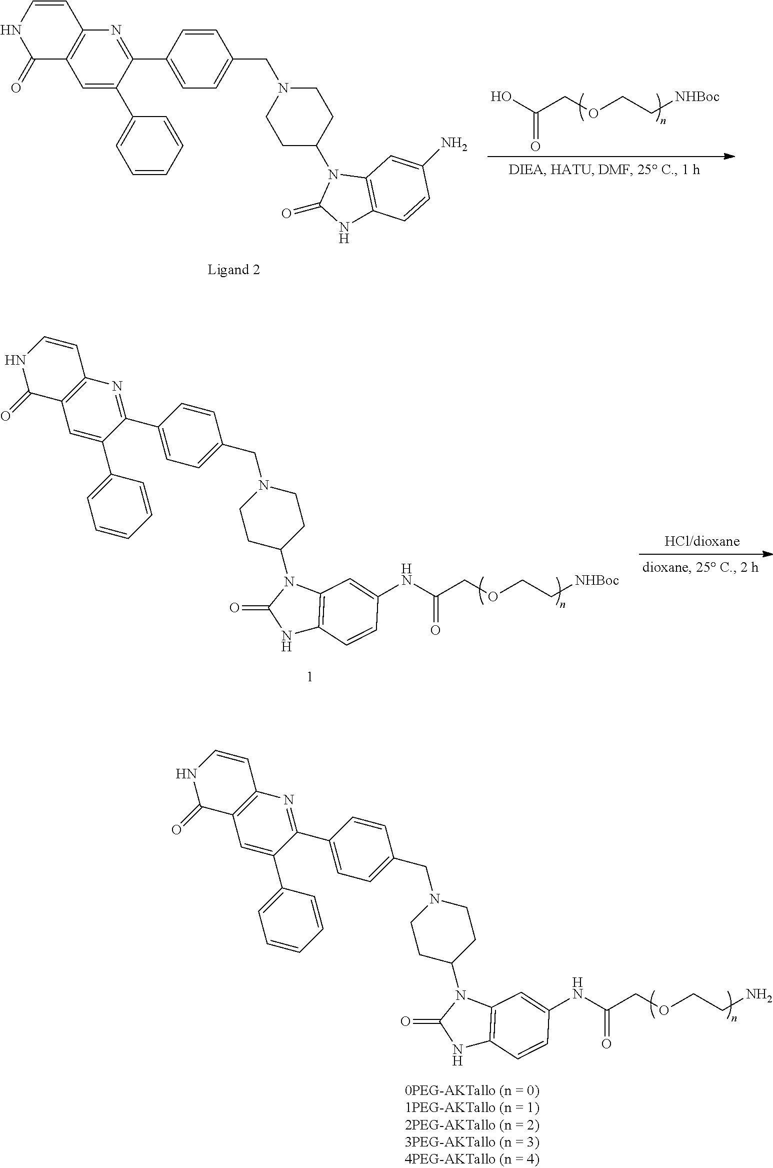

### Part I-General Procedure for Compound 1a-e

To a solution of Ligand 2 (0.5 g, 863 μmol, 1.0 equiv, HCl salt), BocNH-PEG-COOH (863 μmol, 1.0 equiv) and DIEA (223 mg, 1.73 mmol, 300 μL, 2.0 equiv) in DMF (3 mL) was added HATU (492 mg, 1.30 mmol, 1.5 equiv). The mixture was stirred at 25° C. for 1 h. The solution was purified by prep-HPLC on a Waters Xbridge C18 150*50 mm*10 μm column with acetonitrile and NH₄HCO₃ modified water as mobile phase to afford compound 1 as a white solid.
Physical characterization data for compounds 1a-e:
Compound 1a: tert-butyl (2-oxo-2-((2-oxo-3-(1-(4-(5-oxo-3-phenyl-5,6-dihydro-1,6-naphthyridin-2-yl)benzyl)piperidin-4-yl)-2,3-dihydro-1H-benzo[d]imidazol-5-yl)amino)ethyl)carbamate. LC-MS: MS (ES⁺): RT=0.826 min, m/z=700.4 [M+H⁺].
Compound 1b: tert-butyl (2-(2-oxo-2-((2-oxo-3-(1-(4-(5-oxo-3-phenyl-5,6-dihydro-1,6-naphthyridin-2-yl)benzyl)piperidin-4-yl)-2,3-dihydro-1H-benzo[d]imidazol-5-yl)amino)ethoxy)ethyl)carbamate. LC-MS: MS (ES⁺): RT=0.839 min, m/z=744.5 [M+H⁺].
Compound 1c: tert-butyl (2-(2-(2-oxo-2-((2-oxo-3-(1-(4-(5-oxo-3-phenyl-5,6-dihydro-1,6-naphthyridin-2-yl)benzyl)piperidin-4-yl)-2,3-dihydro-1H-benzo[d]imidazol-5-yl)amino)ethoxy)ethoxy)ethyl)carbamate. LC-MS: MS (ES⁺): RT=0.8396 min, m/z=788.5 [M+H⁺].
Compound 1d: tert-butyl (2-(2-(2-(2-oxo-2-((2-oxo-3-(1-(4-(5-oxo-3-phenyl-5,6-dihydro-1,6-naphthyridin-2-yl)benzyl)piperidin-4-yl)-2,3-dihydro-1H-benzo[d]imidazol-5-yl)amino)ethoxy)ethoxy)ethoxy)ethyl)carbamate. LC-MS: MS (ES⁺): RT=0.843 min, m/z=832.5 [M+H⁺].
Compound 1e: tert-butyl (14-oxo-14-((2-oxo-3-(1-(4-(5-oxo-3-phenyl-5,6-dihydro-1,6-naphthyridin-2-yl)benzyl)piperidin-4-yl)-2,3-dihydro-1H-benzo[d]imidazol-5-yl)amino)-3,6,9,12-tetraoxatetradecyl)carbamate. LC-MS: MS (ES⁺): RT=0.849 min, m/z=876.6 [M+H⁺].

### Part II-General Procedure for Compounds (0-4)PEG-AKTallo

To a solution of compound 1a-e (921 μmol, 1.0 equiv) in dioxane (15 mL) was added HCl/dioxane (4 M, 5 mL, 21.7 eq). The mixture was stirred at 25° C. for 2 h and then concentrated to afford compound 2 (HCl salt) as a yellow solid.

### Physical Characterization Data for Compounds (0-4)PEG-AKTallo:

Compound 0PEG-AKTallo (2-amino-N-(2-oxo-3-(1-(4-(5-oxo-3-phenyl-5,6-dihydro-1,6-naphthyridin-2-yl)benzyl)piperidin-4-yl)-2,3-dihydro-1H-benzo[d]imidazol-5-yl)acetamide) LC-MS: MS (ES⁺): RT=0.723 min, m/z=600.5 [M+H⁺]; ¹H NMR (CD₃OD, 400 MHz) δ 9.00 (s, 1H), 7.78 (d, 1H, J=7.5 Hz), 7.72 (d, 2H, J=8.1 Hz), 7.6-7.7 (m, 3H), 7.3-7.4 (m, 3H), 7.2-7.3 (m, 3H), 7.04 (d, 1H, J=8.4 Hz), 6.95 (d, 1H, J=7.6 Hz), 4.6-4.7 (m, 1H), 4.47 (s, 2H), 3.89 (s, 2H), 3.6-3.7 (m, 2H), 2.8-3.0 (m, 2H), 2.09 (m, 2H).
Compound 1PEG-AKTallo (2-(2-aminoethoxy)-N-(2-oxo-3-(1-(4-(5-oxo-3-phenyl-5,6-dihydro-1,6-naphthyridin-2-yl)benzyl)piperidin-4-yl)-2,3-dihydro-1H-benzo[d]imidazol-5-yl)acetamide) LC-MS: MS (ES⁺): RT=0.729 min, m/z=644.5 [M+H⁺]; ¹H NMR (CD₃OD, 400 MHz) δ 8.96 (s, 1H), 7.7-7.8 (m, 4H), 7.62 (d, 2H, J=8.1 Hz), 7.3-7.4 (m, 4H), 7.2-7.3 (m, 2H), 7.04 (d, 1H, J=8.4 Hz), 6.94 (d, 1H, J=7.5 Hz), 4.6-4.7 (m, 1H), 4.46 (s, 2H), 4.24 (s, 2H), 3.8-3.9 (m, 2H), 3.5-3.8 (m, 4H), 3.2-3.3 (m, 2H), 2.8-3.0 (m, 2H), 2.09 (m, 2H).
Compound 2PEG-AKTallo (2-(2-(2-aminoethoxy)ethoxy)-N-(2-oxo-3-(1-(4-(5-oxo-3-phenyl-5,6-dihydro-1,6-naphthyridin-2-yl)benzyl)piperidin-4-yl)-2,3-dihydro-1H-benzo[d]imidazol-5-yl)acetamide) LC-MS: MS (ES⁺): RT=0.730 min, m/z=688.5 [M+H⁺]; ¹H NMR (CD₃OD, 400 MHz) δ 8.87 (s, 1H), 7.7-7.8 (m, 4H), 7.59 (m, 2H), 7.2-7.4 (m, 6H), 7.03 (d, 1H, J=8.4 Hz), 6.94 (d, 1H, J=7.3 Hz), 4.62 (m, 1H), 4.47 (s, 2H), 4.2-4.3 (m, 2H), 3.4-4.0 (m, 10H), 3.16 (m, 2H), 2.8-3.0 (m, 2H), 2.07 (m, 2H).
Compound 3PEG-AKTallo (2-(2-(2-(2-aminoethoxy)ethoxy)ethoxy)-N-(2-oxo-3-(1-(4-(5-oxo-3-phenyl-5,6-dihydro-1,6-naphthyridin-2-yl)benzyl)piperidin-4-yl)-2,3-dihydro-1H-benzo[d]imidazol-5-yl)acetamide) LC-MS: MS (ES⁺): RT=0.743 min, m/z=732.6 [M+H⁺]; ¹H NMR (CD₃OD, 400 MHz) δ 8.96 (s, 1H), 7.75 (d, 2H, J=7.3 Hz), 7.70 (m, 2H), 7.6-7.6 (m, 2H), 7.3-7.4 (m, 3H), 7.3-7.3 (m, 2H), 7.22 (dd, 1H, J=1.3, 8.4 Hz), 7.04 (d, 1H, J=8.3 Hz), 6.94 (d, 1H, J=7.5 Hz), 4.60 (m, 1H), 4.47 (s, 2H), 4.21 (s, 2H), 3.6-3.8 (m, 14H), 3.1-3.2 (m, 2H), 2.8-3.0 (m, 2H), 2.10 (m, 2H).
Compound 4PEG-AKTallo (14-amino-N-(2-oxo-3-(1-(4-(5-oxo-3-phenyl-5,6-dihydro-1,6-naphthyridin-2-yl)benzyl)piperidin-4-yl)-2,3-dihydro-1H-benzo[d]imidazol-5-yl)-3,6,9,12-tetraoxatetradecan-1-amide) LC-MS: MS (ES⁺): RT=0.749 min, m/z=776.6 [M+H⁺]; ¹H NMR (CD₃OD, 400 MHz) δ 8.85 (s, 1H), 7.6-7.7 (m, 6H), 7.3-7.4 (m, 3H), 7.3-7.3 (m, 2H), 7.23 (dd, 1H, J=1.7, 8.4 Hz), 7.04 (d, 1H, J=8.3 Hz), 6.91 (d, 1H, J=7.5 Hz), 4.5-4.6 (m, 1H), 4.44 (s, 2H), 4.22 (s, 2H), 3.6-3.8 (m, 18H), 3.13 (m, 2H), 2.8-3.0 (m, 2H), 2.10 (m, 2H).

### Example 9: Preparation of Compounds (0-4)PEG-AKTcomp (Linker-Target Protein Ligand)

The title compounds were prepared according to the general synthetic procedures below, as described and depicted in the scheme. Preparation of compounds 1 and 4a in the scheme below are described in Blake, J. F., et al. J. Med. Chem. 2012, 55, 8110-8127.

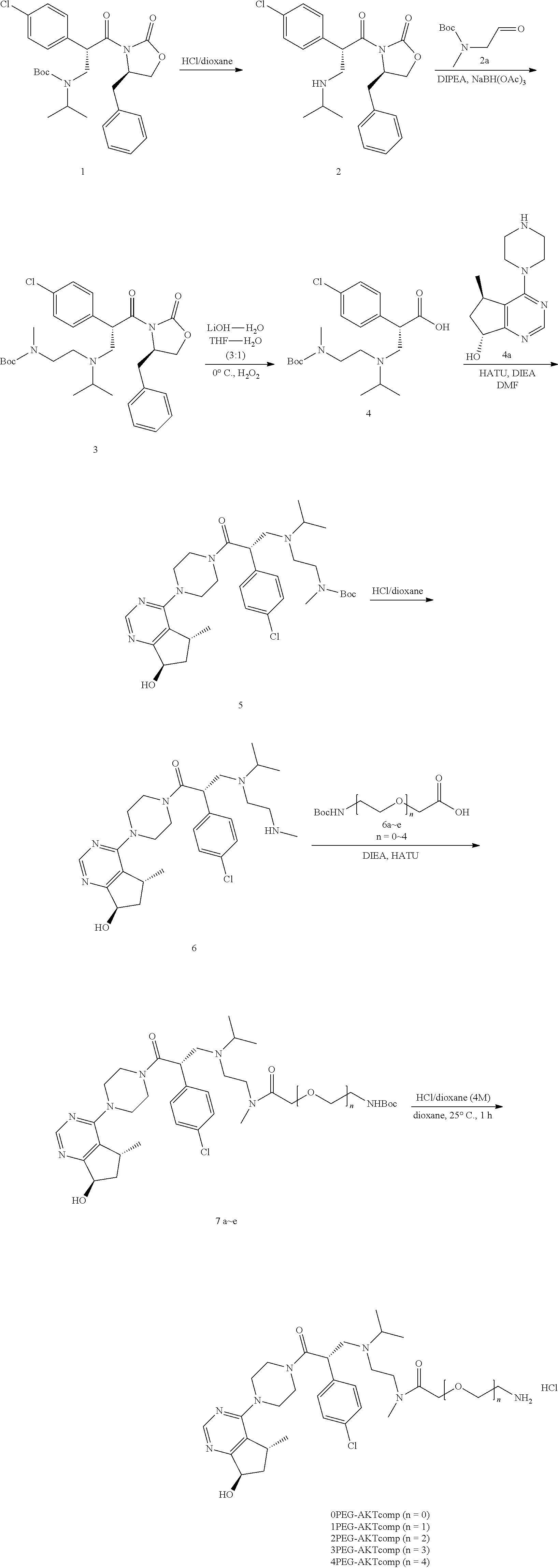

### Part I-Preparation of Compound 2

To a solution of compound 1 (16 g, 32 mmol, 1.0 equiv) in dioxane (50 mL) was added dropwise HCl/dioxane (4 M, 50 mL, 6.3 equiv). The mixture was stirred at 20° C. for 2 h. The reaction mixture was concentrated under reduced pressure to afford compound 2 (13.8 g, crude, HCl salt) as a white solid. 1H NMR (400 MHz, CDCl₃): δ 9.91 (d, 1H, J=6.0 Hz), 9.40 (s, 1H), 7.38 (d, 2H, J=8.5 Hz), 7.28-7.14 (m, 7H), 5.69 (dd, 1H, J=3.8, 9.6 Hz), 4.45-4.53 (m, 1H), 4.01 (d, 1H, J=8.9 Hz), 3.94-3.84 (m, 1H), 3.76-3.68 (m, 2H), 3.30-3.08 (m, 2H), 2.85 (dd, 1H, J=11.5, 13.6 Hz), 1.39 (d, 3H, J=6.5), 1.37 (d, 3H, J=6.5 Hz).

### Part II-Preparation of Compound 3

To a solution of compound 2 (11 g, 25 mmol, 1.0 equiv, HCl salt) in DCE (100 mL) and THF (100 mL) were added DIPEA (9.75 g, 75.5 mmol, 13.1 mL, 3.0 equiv) and compound 2a (4.4 g, 25 mmol, 1.0 equiv), the mixture was stirred for 30 min and then NaBH(OAc)₃ (6.4 g, 30 mmol, 1.2 equiv) was added. The reaction mixture was stirred at 20° C. for 12 h. The mixture was quenched by addition of water (500 mL) and then extracted with ethyl acetate (3×800 mL). The combined organic layers were washed with brine (300 mL), dried over anhydrous sodium sulfate, filtered and concentrated under reduced pressure to give a residue, which was purified via 10 micron Phenomenex Synergi Max-RP C18 250×50 mm column with FA-modified water/acetonitrile mobile phase to afford compound 3 (7.5 g, 53% yield) as a yellow oil. ¹H NMR (400 MHz, CDCl₃): δ 7.41-7.28 (m, 9H), 5.46 (s, 1H), 4.64 (s, 1H), 4.18-4.06 (m, 2H), 3.47-3.34 (m, 2H), 3.25-3.02 (m, 4H), 2.89 (s, 3H), 2.84-2.78 (m, 1H), 2.69 (s, 2H), 1.47 (s, 9H), 1.16-0.96 (m, 6H).

### Part III-Preparation of Compound 4

To a solution of LiOH.H₂O (1.1 g, 25 mmol, 2.0 equiv) in THF (150 mL) and H₂O (50 mL) was added H₂O₂(4.6 g, 37.6 mmol, 3.87 mL, 28% purity, 3 equiv) at 0° C. The solution was stirred for 0.5 h before a solution of compound 3 (7 g, 13 mmol, 1.0 equiv) in THF (50 mL) was added. The mixture was warmed to 20° C. and stirred for 12 h, and then quenched by the addition of Na₂SO₃ aqueous (10 mL) and saturated aqueous NaHCO₃ (10 mL). The mixture was evaporated in vacuo to remove THF. The aqueous solution was acidified to pH 1 with 1 N HCl and then extracted with EtOAc (2×150 mL). The combined organic layers were dried over Na₂SO₄ and concentrated in vacuo to give a residue, which was purified via a Waters XBridge BEH 10 micron 250×50 mm C18 column with ammonia hydroxide modified water/acetonitrile mobile phase to afford compound 4 (1.2 g, 23% yield) as a colorless oil. ¹H NMR (400 MHz, CDCl₃): δ 7.35 (d, 2H, J=8.4 Hz), 7.19 (d, 2H, J=7.7 Hz), 3.65-3.73 (m, 1H), 3.63-3.32 (m, 3H), 3.00-3.20 (m, 2H), 2.93 (s, 3H), 2.68-2.85 (m, 2H), 1.49 (s, 9H), 1.27 (d, 3H, J=6.7 Hz), 1.12 (d, 3H, J=6.5 Hz).

### Part IV-Preparation of Compound 5

A mixture of compound 4 (400 mg, 1.0 mmol, 1.0 equiv), compound 4a (272 mg, 1.0 mmol, 1.0 equiv, HCl salt), HATU (458 mg, 1.2 mmol, 1.2 equiv) and DIEA (778 mg, 6.0 mmol, 1.1 mL, 6.0 equiv) in DMF (3 mL) was stirred at 20° C. for 12 h under N₂ atmosphere. The reaction mixture was concentrated under reduced pressure to give a residue which was purified via a Waters XBridge BEH 10 micron 250×50 mm C18 column with ammonium bicarbonate modified water/acetonitrile mobile phase to afford compound 5 (600 mg, 97% yield) as a white solid. ¹H NMR (400 MHz, CDCl₃): δ 8.51 (s, 1H), 7.15-7.40 (m, 5H), 5.11 (t, 1H, J=7.1 Hz), 4.31 (s, 1H), 3.91-3.61 (m, 5H), 3.57-3.45 (m, 3H), 3.29-3.05 (m, 3H), 2.82 (s, 3H), 2.40-2.65 (s, 2H), 2.23-2.10 (m, 2H), 1.81 (s, 4H), 1.48 (s, 9H), 1.16 (d, 3H, J=6.9 Hz), 0.75-1.05 (m, 6H).

### Part V-Preparation of Compound 6

A mixture of compound 5 (600 mg, 975 μmol, 1.0 equiv) and HCl/dioxane (4 M, 20 mL, 82 equiv) in dioxane (10 mL) was stirred at 20° C. for 2 h under N₂ atmosphere. The reaction mixture was concentrated under reduced pressure to give a residue which was purified via a 15 micron Phenomenex luna C18 150×40 mm column with HCl modified water/actonitrile mobile phase to afford 5 (352 mg, 61% yield, 2 HCl salt) as a white solid. ¹H NMR (400 MHz, CDCl₃): δ 8.60 (s, 1H), 7.51-7.45 (m, 4H), 5.38 (t, 1H, J=7.9 Hz), 4.80 (s, 1H), 4.38-4.21 (m, 1H), 4.14-3.91 (m, 6H), 3.86-3.63 (m, 7H), 3.63-3.63 (m, 1H), 3.46 (s, 1H), 3.24 (s, 1H), 2.88 (s, 3H), 2.37-2.30 (m, 1H), 2.28-2.18 (m, 1H), 1.53 (d, 3H, J=6.4 Hz), 1.44 (d, 3H, J=5.9 Hz), 1.20 (d, 3H, J=5.6 Hz). LC-MS (Method 01): MS (ES⁺): RT=1.93 min, m/z=515.2 [M+H⁺].

### Part VI-General Procedure for the Preparation of Compounds 7a-e

To a solution of 6a (n=0) (697 μmol, 1.0 equiv), 6 (0.4 g, 680 μmol, 1.0 equiv, HCl salt) and DIEA (742 mg, 5.74 mmol, 1.0 mL, 8.4 equiv) in DMF (4 mL) was added HATU (315 mg, 828 μmol, 1.2 equiv). The mixture was then stirred at 25° C. for 12 h, and purified directly on a Waters XBridge 10 micron 150×50 mm C18 column with ammonium bicarbonate modified water/acetonitrile mobile phase to afford 7a (n=0). Compounds 7b-e (n=1, 2, 3, or 4, respectively) are prepared using compound 6b-e (n=1, 2, 3, or 4, respectively) as starting material in lieu of compound 6a.

### Physical Characterization Data for Compounds 7a-e:

Compound 7a (tert-butyl (2-((2-(((S)-2-(4-chlorophenyl)-3-(4-((5R,7R)-7-hydroxy-5-methyl-6,7-dihydro-5H-cyclopenta[d]pyrimidin-4-yl)piperazin-1-yl)-3-oxopropyl)(isopropyl)amino) ethyl)(methyl)amino)-2-oxoethyl)carbamate) LC-MS: MS (ES⁺): RT=1.006 min, m/z=672.4 [M+H⁺].
Compound 7b (tert-butyl (2-(2-((2-(((S)-2-(4-chlorophenyl)-3-(4-((5R,7R)-7-hydroxy-5-methyl-6,7-dihydro-5H-cyclopenta[d]pyrimidin-4-yl)piperazin-1-yl)-3-oxopropyl)(isopropyl)amino) ethyl)(methyl)amino)-2-oxoethoxy)ethyl)carbamate)¹H NMR (400 MHz, CDCl₃) δ 8.52 (s, 1H), 7.15-7.40 (d, 4H), 5.10 (t, 1H, J=7.21 Hz), 4.12-4.19 (m, 2H), 3.57-3.96 (m, 8H), 3.42-3.54 (m, 3H), 3.20-3.40 (m, 6H), 2.85-2.95 (m, 4H), 2.51-2.72 (m, 3H), 2.10-2.22 (m, 2H), 1.45 (s, 9H), 1.16 (dd, 3H, J=6.91, 2.14 Hz), 0.80-1.10 (m, 6H). LC-MS: MS (ES⁺): RT=1.007 m/z=716.4 [M+H⁺].
Compound 7c (tert-butyl ((S)-14-(4-chlorophenyl)-15-(4-((5R,7R)-7-hydroxy-5-methyl-6,7-dihydro-5H-cyclopenta[d]pyrimidin-4-yl)piperazin-1-yl)-12-isopropyl-9-methyl-8,15-dioxo-3,6-dioxa-9,12-diazapentadecyl)carbamate) LC-MS: MS (ES⁺): RT=0.79 min, m/z=760.5 [M+H⁺].
Compound 7d (tert-butyl ((S)-17-(4-chlorophenyl)-18-(4-((5R,7R)-7-hydroxy-5-methyl-6,7-dihydro-5H-cyclopenta[d]pyrimidin-4-yl)piperazin-1-yl)-15-isopropyl-12-methyl-11,18-dioxo-3,6,9-trioxa-12,15-diazaoctadecyl)carbamate) LC-MS: MS (ES⁺): RT=0.989 min, m/z=804.5 [M+H⁺].
Compound 7e (tert-butyl ((S)-20-(4-chlorophenyl)-21-(4-((5R,7R)-7-hydroxy-5-methyl-6,7-dihydro-5H-cyclopenta[d]pyrimidin-4-yl)piperazin-1-yl)-18-isopropyl-15-methyl-14,21-dioxo-3,6,9,12-tetraoxa-15,18-diazahenicosyl)carbamate) LC-MS: MS (ES⁺): RT=1.00 min, m/z=848.4 [M+H⁺].

### Part VII-General Procedure for the Preparation of Compounds (0-4)PEG-AKTcomp

A solution of compound 7a (n=0) (0.2 g, 1.0 equiv) in HCl/dioxane (4 M, 5.0 mL, 116 equiv) was stirred at 25° C. for 1 h, and then the mixture was concentrated to afford (O)PEG-AKTcomp (n=0). Compounds (1-4)PEG-AKTcomp (n=1, 2, 3, or 4, respectively) are prepared using compound 7b-e (n=1, 2, 3, or 4, respectively) as starting material in lieu of compound 7a.

### Physical Characterization Data for Compounds (0-4)PEG-AKTcomp:

Compound 0PEG-AKTcomp (2-amino-N-(2-(((S)-2-(4-chlorophenyl)-3-(4-((5R,7R)-7-hydroxy-5-methyl-6,7-dihydro-5H-cyclopenta[d]pyrimidin-4-yl)piperazin-1-yl)-3-oxopropyl)(isopropyl)amino)ethyl)-N-methylacetamide hydrochloride) LC-MS: MS (ES⁺): RT=0.69 min, m/z=286.9 [M/2+H⁺], 572.2 [M+H⁺].
Compound 1PEG-AKTcomp (2-(2-aminoethoxy)-N-(2-(((S)-2-(4-chlorophenyl)-3-(4-((5R,7R)-7-hydroxy-5-methyl-6,7-dihydro-5H-cyclopenta[d]pyrimidin-4-yl)piperazin-1-yl)-3-oxopropyl)(isopropyl)amino)ethyl)-N-methylacetamide hydrochloride) LC-MS: MS (ES⁺): RT=0.794 min, m/z=308.7 [M/2+H⁺], 616.3 [M+H⁺].
Compound 2PEG-AKTcomp (2-(2-(2-aminoethoxy)ethoxy)-N-(2-(((S)-2-(4-chlorophenyl)-3-(4-((5R,7R)-7-hydroxy-5-methyl-6,7-dihydro-5H-cyclopenta[d]pyrimidin-4-yl)piperazin-1-yl)-3-oxopropyl)(isopropyl)amino)ethyl)-N-methylacetamide hydrochloride) LC-MS: MS (ES⁺): RT=1.027 min, m/z=660.3 [M+H⁺].
Compound 3PEG-AKTcomp (2-(2-(2-(2-aminoethoxy)ethoxy)ethoxy)-N-(2-(((S)-2-(4-chlorophenyl)-3-(4-((5R,7R)-7-hydroxy-5-methyl-6,7-dihydro-5H-cyclopenta[d]pyrimidin-4-yl)piperazin-1-yl)-3-oxopropyl)(isopropyl)amino)ethyl)-N-methylacetamide hydrochloride)¹H NMR (400 MHz, CD₃OD): δ 8.61 (s, 1H), 7.45-7.55 (m, 4H), 5.34 (t, 1H, J=7.95 Hz), 4.99 (s, 1H), 4.34-4.40 (m, 2H), 3.86-4.06 (m, 7H), 3.66-3.62 (m, 16H), 3.38-3.58 (m, 3H), 3.10-3.22 (m, 5H), 2.29-2.36 (m, 1H), 2.16-2.26 (m, 1H), 1.55 (d, 2H, J=6.48 Hz), 1.43 (d, 2H, J=6.48 Hz), 1.36 (d, 2H, J=6.60 Hz), 1.21 (s, 3H). LC-MS: MS (ES⁺): RT=0.511 min, m/z=353.7 [M/2+H⁺], 704.2 [M+H⁺].
Compound 4PEG-AKTcomp (14-amino-N-(2-(((S)-2-(4-chlorophenyl)-3-(4-((5R,7R)-7-hydroxy-5-methyl-6,7-dihydro-5H-cyclopenta[d]pyrimidin-4-yl)piperazin-1-yl)-3-oxopropyl)(isopropyl)amino)ethyl)-N-methyl-3,6,9,12-tetraoxatetradecan-1-amide hydrochloride) LC-MS: MS (ES⁺): RT=1.130 min, m/z=748.4 [M+H⁺].

### Example 10: Preparation of Compounds (0-4)PEG-TBK1 (Linker-Target Protein Ligand)

The title compounds were prepared according to the general synthetic procedures below, as described and depicted in the scheme. Preparation of compound 1 in the scheme below is described in Crew, A. P. et al. J. Med. Chem. 2018, 61, 583-598.

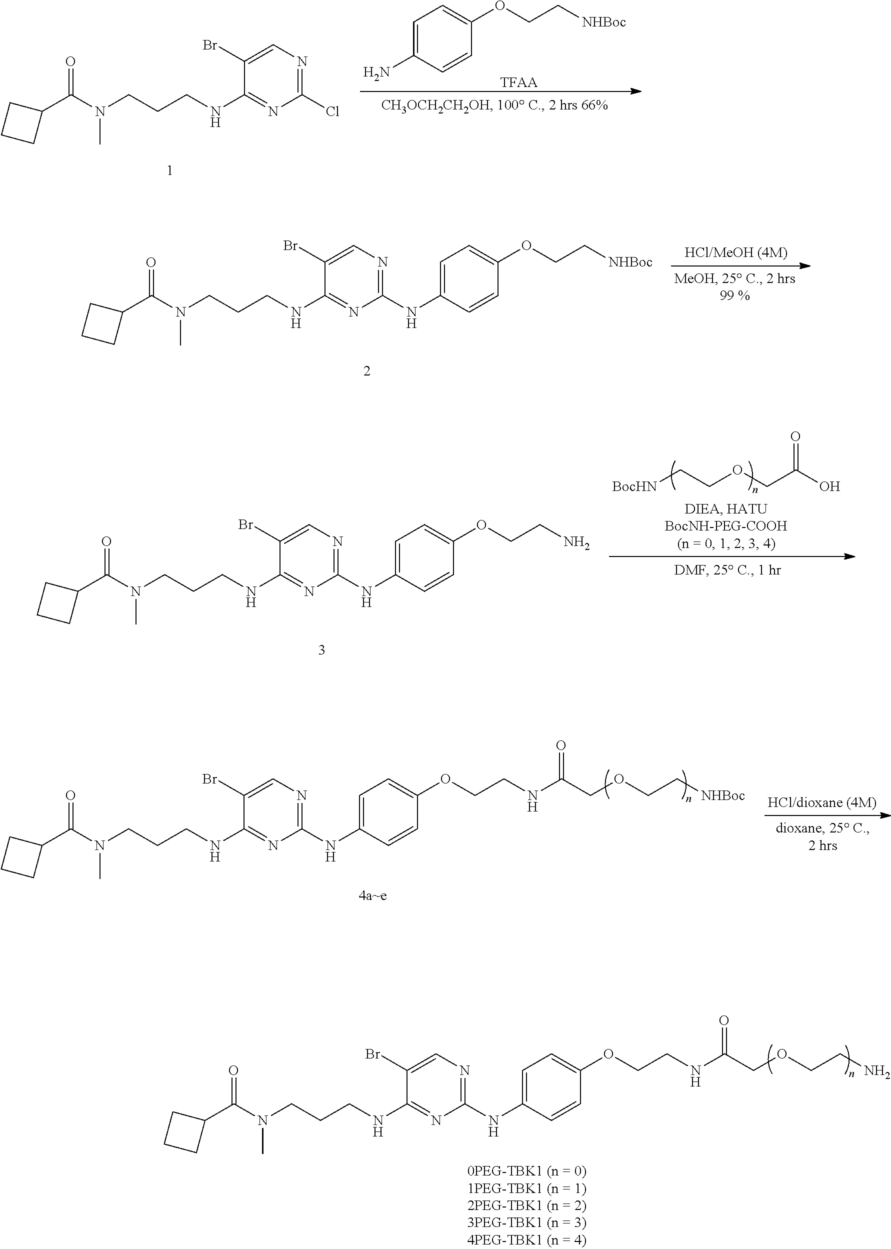

### Part I-Preparation of Compound 2 (tert-butyl N-[2-[4-[[5-bromo-4-[3-[cyclobutanecarbonyl(methyl)amino]propylamino]pyrimidin-2-yl]amino]phenoxy]ethyl]carbamate)

To a solution of 1 (2.87 g, 7.93 mmol, 1 eq), tert-butyl N-[2-(4-aminophenoxy)ethyl]carbamate (2 g, 7.93 mmol, 1 eq) in 2-methoxyethanol (20 mL) was added TFAA (166.49 mg, 792.68 μmol, 110.26 μL, 0.1 eq), and then it was stirred at 100° C. for 2 hrs. The reaction mixture was concentrated to afford crude product. The residue was purified by prep-HPLC (column: Waters X bridge BEH C18 250*50 mm*10 μm; mobile phase: [water (0.05% ammonia hydroxide v/v)-ACN]; B %: 52%-72%, 20 min) to afford compound 2 (3 g, 5.19 mmol, 65.53% yield) as a brown foam. ¹H NMR (400 MHz, CDCl₃): δ 7.90 (s, 1H), 7.40-7.51 (m, 2H), 6.82-6.93 (m, 2H), 6.66-6.81 (m, 1H), 4.95-5.07 (m, 1H), 4.01 (t, J=5.01 Hz, 2H), 3.41-3.56 (m, 6H), 3.18-3.36 (m, 1H), 2.30-2.42 (m, 2H), 2.14-2.26 (m, 2H), 1.73-2.04 (m, 4H), 1.46 (s, 9H). LCMS: MS (ESI): m/z=577.2 [M+H]⁺.

### Part II-Preparation of Compound 3 (N-[3-[[2-[4-(2-aminoethoxy)anilino]-5-bromo-pyrimidin-4-yl]amino]propyl]-N-methyl-cyclobutanecarboxamide)

To a solution of 2 (6 g, 10.39 mmol, 1 eq) in MeOH (60 mL) was added HCl/MeOH (4 M, 20 mL, 7.70 eq), and then it was stirred at 25° C. for 2 hrs. The reaction mixture was concentrated to afford crude product. Compound 3 (5.3 g, 10.31 mmol, 99.28% yield, HCl salt) was obtained as a yellow solid and used for the next step directly. ¹H NMR (400 MHz, CD₃OD): δ 7.94-8.04 (m, 1H), 7.37-7.48 (m, 2H), 7.08-7.17 (m, 2H), 4.21-4.34 (m, 2H), 3.45-3.56 (m, 2H), 3.36-3.45 (m, 4H), 2.74-2.97 (m, 3H), 1.92-2.32 (m, 5H), 1.73-1.91 (m, 3H). LCMS: MS (ESI): m/z=477.1 [M+H]⁺.

### Part III-General Procedure for the Preparation of Compounds 4a-e

To a solution of BocNH-PEG-COOH (n=0,1,2,3,4) (778.43 μmol, 1 eq), 3 (0.4 g, 778.43 μmol, 1 eq, HCl) and DIEA (27.17 mg, 210.19 μmol, 36.61 μL, 2 eq) in DMF (3 mL) was added HATU (443.97 mg, 1.17 mmol, 1.5 eq), and then it was stirred at 25° C. for 2 hrs. The reaction mixture was purified by prep-HPLC (column: Phenomenex luna C18 150*25 mm*10 μm; mobile phase: [water(0.1% TFA)-ACN]; B %: 37%-67%, 10 min) to afford compound 4a-e (n=0,1,2,3,4) as a white solid.

### Part IV-General Procedure for the Preparation of Compounds (0-4)PEG-TBK1

To a solution of 4a-e (0.2 g, 1 eq) in dioxane (3 mL) was added HCl/dioxane (4 M, 1.00 mL, 12.69 eq), and then it was stirred at 25° C. for 2 hrs. The reaction mixture was concentrated to afford crude product. (0-4)PEG-TBK1 were obtained as HCl salts and used for the next step directly.

### Example 11: Preparation of Amide Compounds from Carboxylic Acid-Containing (or Ester-Containing) Protein Phosphatase Ligands and Amine-Containing Linker-Target Protein Ligands

The amide compounds in Table 1 below were prepared from carboxylic acid-containing (or ester-containing) protein phosphatase ligands and amine-containing linker-target protein ligands according to the general synthetic procedures below, as described and depicted in the schemes.

### Part I-General Procedure for Amide Coupling of Carboxylic Acid-Containing Protein Phosphatase Ligands

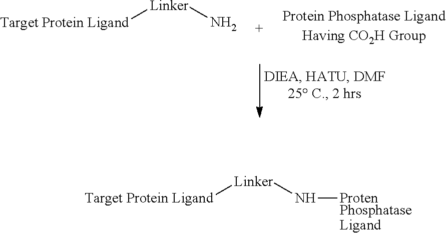

To a solution of carboxylic acid-containing protein phosphatase ligand (1 eq), amine-containing linker-target protein ligand (1 eq, HCl) and DIEA (2 eq) in DMF (2 mL for a typical of 0.1 mmole scale reaction) was added HATU (1.5 eq), and then stirred at 25° C. for 2 hrs. The reaction mixture was neutralized with HOAc, and the mixture was concentrated. The resulting residue was purified by prep-HPLC to afford the product amide compound (e.g., in the form of a TFA salt) as a white solid.

### Part II-General Procedure for Deprotection of Acid-Labile Protecting Groups

For protein phosphatase ligands that contain acid-labile protecting groups (e.g., the pbf-protected guanidine and tBu-protected hydroxyl in Ac-RVSF, Oct-RVSF, and H₂N-RVSF), the amide coupling procedure described in Part I is followed by a TFA deprotection:
To a solution of protected amide compound (1 eq, TFA salt) in TFA (2 mL) was added H₂O (0.2 mL), and then it was stirred at 25° C. for 1 hr. The reaction mixture was concentrated to afford crude product. The residue was purified by prep-HPLC (HCl condition) to afford the product amide compound (HCl salt) as a white solid.
The following scheme depicts the coupling and deprotection procedures for carboxylic acid-containing protein phosphatase ligand Ac-RVSF and amine-containing linker-target protein ligand 1PEG-TBK1 to afford Compound I-111.

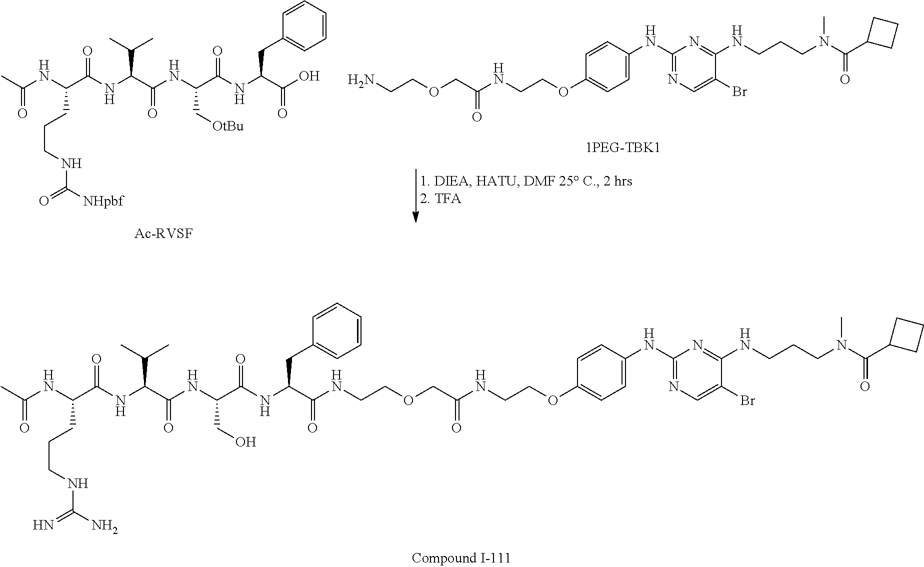

### Physical Characterization Data for Compound I-111:

N-(3-((2-((4-(((6S,9S,12 S,15 S)-6-acetamido-1-amino-15-benzyl-12-(hydroxymethyl)-1-imino-9-isopropyl-7,10,13,16,22-pentaoxo-20-oxa-2,8,11,14,17,23-hexaazapentacosan-25-yl)oxy)phenyl)amino)-5-bromopyrimidin-4-yl)amino)propyl)-N-methylcyclobutanecarboxamide. ¹H NMR (400 MHz, CD₃OD): δ 7.89-7.98 (m, 1H), 7.32-7.41 (m, 2H), 7.14-7.30 (m, 5H), 6.99-7.08 (m, 2H), 4.57 (dd, J=8.32, 6.07 Hz, 1H), 4.33-4.45 (m, 2H), 4.09-4.21 (m, 3H), 3.87-4.00 (m, 2H), 3.75-3.81 (m, 1H), 3.63-3.73 (m, 3H), 3.33-3.55 (m, 8H), 3.10-3.28 (m, 4H), 2.96 (dd, J=13.95, 8.57 Hz, 1H), 2.79-2.92 (m, 3H), 1.90-2.30 (m, 9H), 1.76-1.89 (m, 4H), 1.51-1.75 (m, 3H), 0.92 (dd, J=6.75, 3.13 Hz, 6H). LCMS: MS (ELSD): m/z=1111 [M+H]⁺.

### Part III-General Procedure for Fmoc Deprotection

For protein phosphatase ligands that contain Fmoc protecting groups (e.g. H₂N-RVSF), the Fmoc group(s) are deprotected after the coupling procedure described in Part I and before the TFA deprotection described in Part II (if applicable), using the following procedure: Piperidine (2 eq) was added to the Fmoc-protected amide compound in DMF, and the mixture was stirred for 1 hr. The reaction mixture was neutralized with HOAc, and the mixture was concentrated. The resulting residue was purified by prep-HPLC to afford free amine (TFA salt) as a white solid.
The following scheme depicts the deprotection procedure for an Fmoc-protected amide compound synthesized from protein phosphatase ligand H₂N-RVSF.

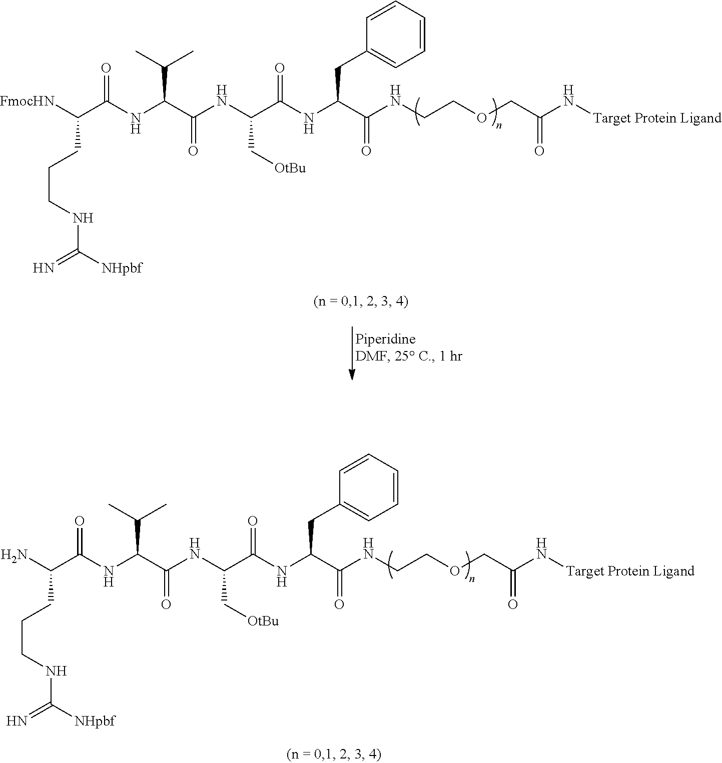

Part IV-General Procedure for Amide Formation with Ester-Containing Protein phosphatase ligands
For protein phosphatase ligands with activated ester moieties (e.g., the difluoromethylaryloxy ester in SMAP-3DiF), the following amide coupling procedure was used: A mixture of ester-containing protein phosphatase ligand (˜120 μmol, 1 equiv), amine-containing linker-target protein ligand (1 equiv) and DIPEA (2 equiv) in anhydrous DMF (1 mL) was degassed and purged with N₂ three times. The mixture was stirred at 25° C. for 3 h under N₂ atmosphere and then concentrated to give a residue. The residue was purified by prep-HPLC to afford the product amide compound.
The following scheme depicts the coupling procedure for ester-containing protein phosphatase ligand SMAP-3DiF (70 mg) and amine-containing linker-target protein ligand 0PEG-Chloroalkane, which afforded Compound I-31 (30 mg, 30% yield) after purification by prep-HPLC (column: Waters Xbridge C18 150*50 mm*10 μm; mobile phase: [water (10 mM NH₄HCO₃)-ACN]; B %: 55%-85%, 10 min).

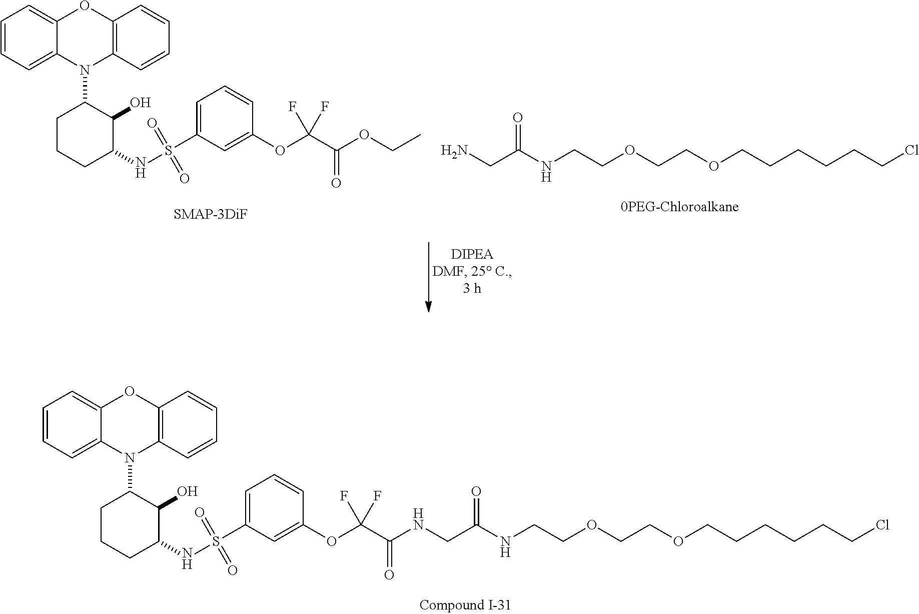

### Physical Characterization Data for Compound I-31:

N-(2-((2-(2-((6-chlorohexyl)oxy)ethoxy)ethyl)amino)-2-oxoethyl)-2,2-difluoro-2-(3-(N-((1R,2R,3 S)-2-hydroxy-3-(10H-phenoxazin-10-yl)cyclohexyl)sulfamoyl)phenoxy)acetamide. ¹H NMR (400 MHz, CDCl₃) δ 7.89 (s, 1H), 7.83-7.81 (m, 1H), 7.57-7.75 (m, 1H), 7.49-7.56 (m, 1H), 7.39-7.47 (m, 1H), 6.83-6.93 (m, 6H), 6.81-6.79 (m, 2H), 3.77-3.93 (m, 3H), 3.53-3.62 (m, 6H), 3.45-3.53 (m, 4H), 3.28-3.43 (m, 3H), 3.04-3.20 (m, 1H), 2.13-2.11 (m, 1H), 1.99-1.97 (m, 1H), 1.68-1.86 (m, 7H), 1.60-1.66 (m, 2H), 1.27-1.55 (m, 6H). LC-MS: MS (ES⁺): RT=2.72 min, m/z=809.2 [M+H⁺]; 405.2 [M/2+H⁺].

### Part V-Exemplary Amide Compounds Prepared According to the General Procedures

Product amide compounds in Table 1 below were prepared from carboxylic acid-containing (or ester-containing) protein phosphatase ligands and amine-containing linker-target protein ligands according to the procedures described in Parts I-IV herein. Chemical structures for abbreviations used in the description of product amide compounds in Table 1 are provided in Table 2 below. Protein phosphatase ligand 1H4 is commercially available (CAS RN 379726-67-7) and has the
chemical structure

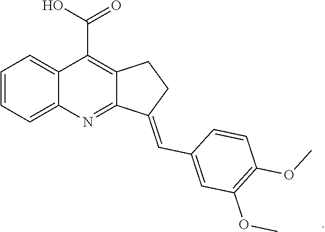 [SMILES: COc1ccc(/C=C2\CCc3c2nc2ccccc2c3C(=O)O)cc1OC]

TABLE 1
Exemplary Product Amide Compounds

| | | Protein | | | | | |
|---|---|---|---|---|---|---|---|
| | | Phosphatase | | | | | |
| | | Ligand Used | Linker-Target | | | | |
| | | to Prepare | Protein Ligand | | | | |
| Com- | | Product | Used to Prepare | | LC/MS | | |
| pound | Product Amide | Amide | Product Amide | | MH + | | |
| No. | Compound | Compound | Compound | MW | (obs.) | RT | Method |
| I-1 | (Ac-RVSF)-(0PEG- | Ac-RVSF | 0PEG- | 812 | 812 | 2.43 | 01 |
| | Chloroalkane) | | Chloroalkane | | | | |
| I-2 | (Ac-RVSF)-(1PEG- | Ac-RVSF | 1PEG- | 856 | 856 | 2.46 | 01 |
| | Chloroalkane) | | Chloroalkane | | | | |
| I-3 | (Ac-RVSF)-(2PEG- | Ac-RVSF | 2PEG- | 901 | 900 | 2.46 | 01 |
| | Chloroalkane) | | Chloroalkane | | | | |
| I-4 | (Ac-RVSF)-(3PEG- | Ac-RVSF | 3PEG- | 945 | 944 | 2.49 | 01 |
| | Chloroalkane) | | Chloroalkane | | | | |
| I-5 | (Ac-RVSF)-(4PEG- | Ac-RVSF | 4PEG- | 989 | 988 | 2.52 | 01 |
| | Chloroalkane) | | Chloroalkane | | | | |
| I-6 | (Oct-RVSF)-(0PEG- | Oct-RVSF | 0PEG- | 897 | 896 | 2.78 | 01 |
| | Chloroalkane) | | Chloroalkane | | | | |
| I-7 | (Oct-RVSF)-(1PEG- | Oct-RVSF | 1PEG- | 941 | 940 | 2.83 | 01 |
| | Chloroalkane) | | Chloroalkane | | | | |
| I-8 | (Oct-RVSF)-(2PEG- | Oct-RVSF | 2PEG- | 985 | 984 | 2.80 | 01 |
| | Chloroalkane) | | Chloroalkane | | | | |
| I-9 | (Oct-RVSF)-(3PEG- | Oct-RVSF | 3PEG- | 1029 | 515 | 2.82 | 01 |
| | Chloroalkane) | | Chloroalkane | | [M/2 + H] | | |
| I-10 | (Oct-RVSF)-(4PEG- | Oct-RVSF | 4PEG- | 1073 | 537 | 2.83 | 01 |
| | Chloroalkane) | | Chloroalkane | | [M/2 + H] | | |
| I-11 | (H₂N-RVSF)-(0PEG- | H₂N-RVSF | 0PEG- | 770 | 770 | 2.00 | 10 |
| | Chloroalkane) | | Chloroalkane | | | | |
| I-12 | (H₂N-RVSF)-(1PEG- | H₂N-RVSF | 1PEG- | 814 | 814 | 2.01 | 10 |
| | Chloroalkane) | | Chloroalkane | | | | |
| I-13 | (H₂N-RVSF)-(2PEG- | H₂N-RVSF | 2PEG- | 858 | 858 | 2.04 | 10 |
| | Chloroalkane) | | Chloroalkane | | | | |
| I-14 | (H₂N-RVSF)-(3PEG- | H₂N-RVSF | 3PEG- | 903 | 902 | 2.05 | 10 |
| | Chloroalkane) | | Chloroalkane | | | | |
| I-15 | (H₂N-RVSF)-(4PEG- | H₂N-RVSF | 4PEG- | 947 | 946 | 2.09 | 10 |
| | Chloroalkane) | | Chloroalkane | | | | |
| I-16 | (1H4)-(0PEG- | 1H4 | 0PEG- | 624 | 624 | 2.52 | 10 |
| | Chloroalkane) | | Chloroalkane | | | | |
| I-17 | (1H4)-(1PEG- | 1H4 | 1PEG- | 668 | 668 | 2.12 | 25 |
| | Chloroalkane) | | Chloroalkane | | | | |
| I-18 | (1H4)-(2PEG- | 1H4 | 2PEG- | 712 | 712 | 2.15 | 25 |
| | Chloroalkane) | | Chloroalkane | | | | |
| I-19 | (1H4)-(3PEG- | 1H4 | 3PEG- | 756 | 756 | 2.57 | 10 |
| | Chloroalkane) | | Chloroalkane | | | | |
| I-20 | (1H4)-(4PEG- | 1H4 | 4PEG- | 800 | 800 | 2.57 | 10 |
| | Chloroalkane) | | Chloroalkane | | | | |
| I-21 | (SMAP-Direct)-(0PEG- | SMAP-Direct | 0PEG- | 743 | 743 | 2.54 | 25 |
| | Chloroalkane) | | Chloroalkane | | | | |
| I-22 | (SMAP-Direct)-(1PEG- | SMAP-Direct | 1PEG- | 787 | 787 | 2.58 | 25 |
| | Chloroalkane) | | Chloroalkane | | | | |
| I-23 | (SMAP-Direct)-(2PEG- | SMAP-Direct | 2PEG- | 831 | 831 | 2.59 | 25 |
| | Chloroalkane) | | Chloroalkane | | | | |
| I-24 | (SMAP-Direct)-(3PEG- | SMAP-Direct | 3PEG- | 875 | 875 | 2.61 | 25 |
| | Chloroalkane) | | Chloroalkane | | | | |
| I-25 | (SMAP-Direct)-(4PEG- | SMAP-Direct | 4PEG- | 920 | 919 | 2.60 | 25 |
| | Chloroalkane) | | Chloroalkane | | | | |
| I-26 | (SMAP-4DiF)-(0PEG- | SMAP-4DiF | 0PEG- | 809 | 809 | 2.68 | 25 |
| | Chloroalkane) | | Chloroalkane | | | | |
| I-27 | (SMAP-4DiF)-(1PEG- | SMAP-4DiF | 1PEG- | 853 | 853 | 2.73 | 25 |
| | Chloroalkane) | | Chloroalkane | | | | |
| I-28 | (SMAP-4DiF)-(2PEG- | SMAP-4DiF | 2PEG- | 897 | 897 | 2.74 | 25 |
| | Chloroalkane) | | Chloroalkane | | | | |
| I-29 | (SMAP-4DiF)-(3PEG- | SMAP-4DiF | 3PEG- | 941 | 941 | 2.75 | 25 |
| | Chloroalkane) | | Chloroalkane | | | | |
| I-30 | (SMAP-4DiF)-(4PEG- | SMAP-4DiF | 4PEG- | 986 | 985 | 2.74 | 25 |
| | Chloroalkane) | | Chloroalkane | | | | |
| I-31 | (SMAP-3DiF)-(0PEG- | SMAP-3DiF | 0PEG- | 809 | 809 | 2.72 | 25 |
| | Chloroalkane) | | Chloroalkane | | | | |
| I-32 | (SMAP-3DiF)-(1PEG- | SMAP-3DiF | 1PEG- | 853 | 853 | 2.74 | 25 |
| | Chloroalkane) | | Chloroalkane | | | | |
| I-33 | (SMAP-3DiF)-(2PEG- | SMAP-3DiF | 2PEG- | 897 | 897 | 2.73 | 25 |
| | Chloroalkane) | | Chloroalkane | | | | |
| I-34 | (SMAP-3DiF)-(3PEG- | SMAP-3DiF | 3PEG- | 941 | 941 | 2.76 | 25 |
| | Chloroalkane) | | Chloroalkane | | | | |
| I-35 | (JNS 1-40)-(0PEG- | JNS 1-40 | 0PEG- | 655 | 654 | 2.66 | 10 |
| | Chloroalkane) | | Chloroalkane | | | | |
| I-36 | (JNS 1-40)-(1PEG- | JNS 1-40 | 1PEG- | 699 | 698 | 2.70 | 10 |
| | Chloroalkane) | | Chloroalkane | | | | |
| I-37 | (JNS 1-40)-(2PEG- | JNS 1-40 | 2PEG- | 743 | 742 | 2.70 | 10 |
| | Chloroalkane) | | Chloroalkane | | | | |
| I-38 | (JNS I-40)-(3PEG- | JNS 1-40 | 3PEG- | 787 | 786 | 2.72 | 10 |
| | Chloroalkane) | | Chloroalkane | | | | |
| I-39 | (JNS 1-40)-(4PEG- | JNS 1-40 | 4PEG- | 831 | 830 | 2.73 | 10 |
| | Chloroalkane) | | Chloroalkane | | | | |
| I-40 | (Ac-RVSF)-(0PEG-AKTallo) | Ac-RVSF | 0PEG-AKTallo | 1131 | 1131 | 2.26 | 01 |
| I-41 | (Ac-RVSF)-(1PEG-AKTallo) | Ac-RVSF | 1PEG-AKTallo | 1175 | 1176 | 2.28 | 01 |
| I-42 | (Ac-RVSF)-(2PEG-AKTallo) | Ac-RVSF | 2PEG-AKTallo | 1219 | 1220 | 2.30 | 01 |
| I-43 | (Ac-RVSF)-(3PEG-AKTallo) | Ac-RVSF | 3PEG-AKTallo | 1263 | 1264 | 2.32 | 01 |
| I-44 | (Ac-RVSF)-(4PEG-AKTallo) | Ac-RVSF | 4PEG-AKTallo | 1308 | 1308 | 2.01 | 10 |
| I-45 | (Oct-RVSF)-(0PEG-AKTallo) | Oct-RVSF | 0PEG-AKTallo | 1215 | 1215 | 2.24 | 10 |
| I-46 | (Oct-RVSF)-(1PEG-AKTallo) | Oct-RVSF | 1PEG-AKTallo | 1260 | 1259 | 2.24 | 10 |
| I-47 | (Oct-RVSF)-(2PEG-AKTallo) | Oct-RVSF | 2PEG-AKTallo | 1304 | 1305 | 2.26 | 10 |
| I-48 | (Oct-RVSF)-(3PEG-AKTallo) | Oct-RVSF | 3PEG-AKTallo | 1348 | 1347 | 2.27 | 10 |
| I-49 | (Oct-RVSF)-(4PEG-AKTallo) | Oct-RVSF | 4PEG-AKTallo | 1392 | 1392 | 2.61 | 01 |
| I-50 | (H₂N-RVSF)-(0PEG-AKTallo) | H₂N-RVSF | 0PEG-AKTallo | 1089 | 1090 | 2.17 | 01 |
| I-51 | (H₂N-RVSF)-(1PEG-AKTallo) | H₂N-RVSF | 1PEG-AKTallo | 1133 | 1133 | 2.19 | 01 |
| I-52 | (H₂N-RVSF)-(2PEG-AKTallo) | H₂N-RVSF | 2PEG-AKTallo | 1177 | 1178 | 2.22 | 01 |
| I-53 | (H₂N-RVSF)-(3PEG-AKTallo) | H₂N-RVSF | 3PEG-AKTallo | 1221 | 1221 | 2.24 | 01 |
| I-54 | (H₂N-RVSF)-(4PEG-AKTallo) | H₂N-RVSF | 4PEG-AKTallo | 1265 | 1265 | 2.25 | 01 |
| I-55 | (SMAP-Direct)-(0PEG- | SMAP-Direct | 0PEG-AKTallo | 1062 | 1063 | 2.11 | 25 |
| | AKTallo) | | | | | | |
| I-56 | (SMAP-Direct)-(1PEG- | SMAP-Direct | 1PEG-AKTallo | 1106 | 1106 | 2.53 | 10 |
| | AKTallo) | | | | | | |
| I-57 | (SMAP-Direct)-(2PEG- | SMAP-Direct | 2PEG-AKTallo | 1150 | 1150 | 2.15 | 25 |
| | AKTallo) | | | | | | |
| I-58 | (SMAP-Direct)-(3PEG- | SMAP-Direct | 3PEG-AKTallo | 1194 | 1194 | 2.17 | 25 |
| | AKTallo) | | | | | | |
| I-59 | (SMAP-Direct)-(4PEG- | SMAP-Direct | 4PEG-AKTallo | 1238 | 1237 | 2.18 | 25 |
| | AKTallo) | | | | | | |
| I-60 | (SMAP-4DiF)-(0PEG-AKTallo) | SMAP-4DiF | 0PEG-AKTallo | 1128 | 1128 | 2.24 | 25 |
| I-61 | (SMAP-4DiF)-(1PEG-AKTallo) | SMAP-4DiF | 1PEG-AKTallo | 1172 | 1172 | 2.26 | 25 |
| I-62 | (SMAP-4DiF)-(2PEG-AKTallo) | SMAP-4DiF | 2PEG-AKTallo | 1216 | 1216 | 2.27 | 25 |
| I-63 | (SMAP-4DiF)-(3PEG-AKTallo) | SMAP-4DiF | 3PEG-AKTallo | 1260 | 1259 | 2.29 | 25 |
| I-64 | (SMAP-4DiF)-(4PEG-AKTallo) | SMAP-4DiF | 4PEG-AKTallo | 1304 | 1304 | 2.31 | 25 |
| I-65 | (SMAP-3DiF)-(0PEG-AKTallo) | SMAP-3DiF | 0PEG-AKTallo | 1128 | 1130 | 2.27 | 25 |
| I-66 | (SMAP-3DiF)-(1PEG-AKTallo) | SMAP-3DiF | 1PEG-AKTallo | 1172 | 1172 | 2.27 | 25 |
| I-67 | (SMAP-3DiF)-(2PEG-AKTallo) | SMAP-3DiF | 2PEG-AKTallo | 1216 | 1218 | 2.28 | 25 |
| I-68 | (SMAP-3DiF)-(3PEG-AKTallo) | SMAP-3DiF | 3PEG-AKTallo | 1260 | 1261 | 2.30 | 25 |
| I-69 | (SMAP-3DiF)-(4PEG-AKTallo) | SMAP-3DiF | 4PEG-AKTallo | 1304 | 1304 | 2.32 | 25 |
| I-70 | (JNS 1-40)-(0PEG-AKTallo) | JNS 1-40 | 0PEG-AKTallo | 973 | 973.3 | 1.90 | 25 |
| I-71 | (JNS 1-40)-(1PEG-AKTallo) | JNS 1-40 | 1PEG-AKTallo | 1018 | 1017 | 1.91 | 25 |
| I-72 | (JNS 1-40)-(2PEG-AKTallo) | JNS 1-40 | 2PEG-AKTallo | 1062 | 1063 | 1.94 | 25 |
| I-73 | (JNS 1-40)-(3PEG- | JNS 1-40 | 3PEG-AKTallo | 1106 | 553 | 1.96 | 25 |
| | AKTallo) | | | | [M/2 + H] | | |
| I-74 | (JNS 1-40)-(4PEG- | JNS 1-40 | 4PEG-AKTallo | 1150 | 575 | 1.97 | 25 |
| | AKTallo) | | | | [M/2 + H] | | |
| I-75 | (Ac-RVSF)-(0PEG-AKTcomp) | Ac-RVSF | 0PEG-AKTcomp | 1104 | 1105 | 2.14 | 01 |
| I-76 | (Ac-RVSF)-(1PEG-AKTcomp) | Ac-RVSF | 1PEG-AKTcomp | 1148 | 1148 | 2.14 | 01 |
| I-77 | (Ac-RVSF)-(2PEG-AKTcomp) | Ac-RVSF | 2PEG-AKTcomp | 1192 | 1193 | 2.16 | 01 |
| I-78 | (Ac-RVSF)-(3PEG-AKTcomp) | Ac-RVSF | 3PEG-AKTcomp | 1236 | 1237 | 2.17 | 01 |
| I-79 | (Ac-RVSF)-(4PEG-AKTcomp) | Ac-RVSF | 4PEG-AKTcomp | 1280 | 1280 | 2.19 | 01 |
| I-80 | (Oct-RVSF)-(0PEG-AKTcomp) | Oct-RVSF | 0PEG-AKTcomp | 1188 | 1188 | 2.13 | 10 |
| I-81 | (Oct-RVSF)-(1PEG-AKTcomp) | Oct-RVSF | 1PEG-AKTcomp | 1232 | 1233 | 2.10 | 10 |
| I-82 | (Oct-RVSF)-(2PEG-AKTcomp) | Oct-RVSF | 2PEG-AKTcomp | 1276 | 1278 | 2.08 | 10 |
| I-83 | (Oct-RVSF)-(3PEG- | Oct-RVSF | 3PEG-AKTcomp | 1320 | 660 | 2.14 | 10 |
| | AKTcomp) | | | | [M/2 + H] | | |
| I-84 | (Oct-RVSF)-(4PEG- | Oct-RVSF | 4PEG-AKTcomp | 1364 | 683 | 2.15 | 10 |
| | AKTcomp) | | | | [M/2 + H] | | |
| I-85 | (H₂N-RVSF)-(0PEG-AKTcomp) | H₂N-RVSF | 0PEG-AKTcomp | 1062 | 1061 | 2.04 | 01 |
| I-86 | (H₂N-RVSF)-(1PEG-AKTcomp) | H₂N-RVSF | 1PEG-AKTcomp | 1106 | 1106 | 2.06 | 01 |
| I-87 | (H₂N-RVSF)-(2PEG-AKTcomp) | H₂N-RVSF | 2PEG-AKTcomp | 1150 | 1149 | 2.05 | 01 |
| I-88 | (H₂N-RVSF)-(3PEG-AKTcomp) | H₂N-RVSF | 3PEG-AKTcomp | 1194 | 1196 | 2.10 | 01 |
| I-89 | (H₂N-RVSF)-(4PEG-AKTcomp) | H₂N-RVSF | 4PEG-AKTcomp | 1238 | 1240 | 2.09 | 01 |
| I-90 | (SMAP-Direct)-(0PEG- | SMAP-Direct | 0PEG-AKTcomp | 1035 | 1036 | 1.92 | 25 |
| | AKTcomp) | | | | | | |
| I-91 | (SMAP-Direct)-(1PEG- | SMAP-Direct | 1PEG-AKTcomp | 1079 | 1078 | 2.69 | 01 |
| | AKTcomp) | | | | | | |
| I-92 | (SMAP-Direct)-(2PEG- | SMAP-Direct | 2PEG-AKTcomp | 1123 | 1125 | 1.94 | 25 |
| | AKTcomp) | | | | | | |
| I-93 | (SMAP-Direct)-(3PEG- | SMAP-Direct | 3PEG-AKTcomp | 1167 | 584 | 1.97 | 25 |
| | AKTcomp) | | | | [M/2 + H] | | |
| I-94 | (SMAP-Direct)-(4PEG- | SMAP-Direct | 4PEG-AKTcomp | 1211 | 606 | 1.96 | 25 |
| | AKTcomp) | | | | [M/2 + H] | | |
| I-95 | (SMAP-4DiF)-(0PEG-AKTcomp) | SMAP-4DiF | 0PEG-AKTcomp | 1101 | 1102 | 2.04 | 25 |
| I-96 | (SMAP-4DiF)-(1PEG-AKTcomp) | SMAP-4DiF | 1PEG-AKTcomp | 1145 | 1147 | 2.02 | 25 |
| I-97 | (SMAP-4DiF)-(2PEG-AKTcomp) | SMAP-4DiF | 2PEG-AKTcomp | 1189 | 1189 | 2.07 | 25 |
| I-98 | (SMAP-4DiF)-(3PEG-AKTcomp) | SMAP-4DiF | 3PEG-AKTcomp | 1233 | 1233 | 2.05 | 25 |
| I-99 | (SMAP-4DiF)-(4PEG- | SMAP-4DiF | 4PEG-AKTcomp | 1277 | 639 | 2.11 | 25 |
| | AKTcomp) | | | | [M/2 + H] | | |
| I-100 | (SMAP-3DiF)-(0PEG-AKTcomp) | SMAP-3DiF | 0PEG-AKTcomp | 1101 | 1100 | 2.73 | 30 |
| I-101 | (SMAP-3DiF)-(1PEG-AKTcomp) | SMAP-3DiF | 1PEG-AKTcomp | 1145 | 1144 | 2.09 | 25 |
| I-102 | (SMAP-3DiF)-(2PEG-AKTcomp) | SMAP-3DiF | 2PEG-AKTcomp | 1189 | 1191 | 2.07 | 25 |
| I-103 | (SMAP-3DiF)-(3PEG-AKTcomp) | SMAP-3DiF | 3PEG-AKTcomp | 1233 | 1233 | 2.10 | 25 |
| I-104 | (SMAP-3DiF)-(4PEG-AKTcomp) | SMAP-3DiF | 4PEG-AKTcomp | 1277 | 1276 | 2.11 | 25 |
| I-105 | (JNS 1-40)-(0PEG-AKTcomp) | JNS 1-40 | 0PEG-AKTcomp | 946 | 945 | 1.66 | 25 |
| I-106 | (JNS 1-40)-(1PEG-AKTcomp) | JNS 1-40 | 1PEG-AKTcomp | 990 | 989 | 1.66 | 25 |
| I-107 | (JNS 1-40)-(2PEG-AKTcomp) | JNS 1-40 | 2PEG-AKTcomp | 1034 | 1033 | 1.69 | 25 |
| I-108 | (JNS 1-40)-(3PEG-AKTcomp) | JNS 1-40 | 3PEG-AKTcomp | 1078 | 1077 | 1.72 | 25 |
| I-109 | (JNS 1-40)-(4PEG- | JNS 1-40 | 4PEG-AKTcomp | 1122 | 561 | 1.74 | 25 |
| | AKTcomp) | | | | [M/2 + H] | | |
| I-110 | (Ac-RVSF)-(0PEG-TBK1) | Ac-RVSF | 0PEG-TBK1 | 1066 | 1066 | 2.29 | 01 |
| I-111 | (Ac-RVSF)-(1PEG-TBK1) | Ac-RVSF | 1PEG-TBK1 | 1110 | 1111 | 2.30 | 01 |
| I-112 | (Ac-RVSF)-(2PEG-TBK1) | Ac-RVSF | 2PEG-TBK1 | 1154 | 1156 | 2.33 | 01 |
| I-113 | (Ac-RVSF)-(3PEG-TBK1) | Ac-RVSF | 3PEG-TBK1 | 1198 | 1199 | 2.35 | 01 |
| I-114 | (Ac-RVSF)-(4PEG-TBK1) | Ac-RVSF | 4PEG-TBK1 | 1242 | 1241 | 2.36 | 01 |
| I-115 | (Oct-RVSF)-(0PEG-TBK1) | Oct-RVSF | 0PEG-TBK1 | 1150 | 1152 | 2.26 | 10 |
| I-116 | (Oct-RVSF)-(1PEG-TBK1) | Oct-RVSF | 1PEG-TBK1 | 1194 | 1195 | 2.28 | 10 |
| I-117 | (Oct-RVSF)-(2PEG- | Oct-RVSF | 2PEG-TBK1 | 1238 | 620 | 2.31 | 10 |
| | TBK1) | | | | [M/2 + H] | | |
| I-118 | (Oct-RVSF)-(3PEG-TBK1) | Oct-RVSF | 3PEG-TBK1 | 1282 | 1284 | 2.31 | 10 |
| I-119 | (Oct-RVSF)-(4PEG-TBK1) | Oct-RVSF | 4PEG-TBK1 | 1326 | 1327 | 2.33 | 10 |
| I-120 | (H₂N-RVSF)-(0PEG-TBK1) | H₂N-RVSF | 0PEG-TBK1 | 1024 | 1025 | 2.18 | 01 |
| I-121 | (H₂N-RVSF)-(1PEG-TBK1) | H₂N-RVSF | 1PEG-TBK1 | 1068 | 1068 | 2.20 | 01 |
| I-122 | (H₂N-RVSF)-(2PEG-TBK1) | H₂N-RVSF | 2PEG-TBK1 | 1112 | 1111 | 2.23 | 01 |
| I-123 | (H₂N-RVSF)-(3PEG-TBK1) | H₂N-RVSF | 3PEG-TBK1 | 1156 | 1158 | 2.25 | 01 |
| I-124 | (H₂N-RVSF)-(4PEG-TBK1) | H₂N-RVSF | 4PEG-TBK1 | 1200 | 1199 | 2.27 | 01 |
| I-125 | (SMAP-Direct)-(0PEG- | SMAP-Direct | 0PEG-TBK1 | 997 | 996 | 2.19 | 25 |
| | TBK1) | | | | | | |
| I-126 | (SMAP-Direct)-(1PEG- | SMAP-Direct | 1PEG-TBK1 | 1041 | 1040 | 2.20 | 25 |
| | TBK1) | | | | | | |
| I-127 | (SMAP-Direct)-(2PEG- | SMAP-Direct | 2PEG-TBK1 | 1085 | 1086 | 2.22 | 25 |
| | TBK1) | | | | | | |
| I-128 | (SMAP-Direct)-(3PEG- | SMAP-Direct | 3PEG-TBK1 | 1129 | 1130 | 2.23 | 25 |
| | TBK1) | | | | | | |
| I-129 | (SMAP-Direct)-(4PEG- | SMAP-Direct | 4PEG-TBK1 | 1173 | 587 | 2.25 | 25 |
| | TBK1) | | | | [M/2 + H] | | |
| I-130 | (SMAP-4DiF)-(0PEG-TBK1) | SMAP-4DiF | 0PEG-TBK1 | 1063 | 1061 | 2.31 | 25 |
| I-131 | (SMAP-4DiF)-(1PEG-TBK1) | SMAP-4DiF | 1PEG-TBK1 | 1107 | 1106 | 2.31 | 25 |
| I-132 | (SMAP-4DiF)-(2PEG-TBK1) | SMAP-4DiF | 2PEG-TBK1 | 1151 | 1153 | 2.34 | 25 |
| I-133 | (SMAP-4DiF)-(3PEG-TBK1) | SMAP-4DiF | 3PEG-TBK1 | 1195 | 1198 | 2.34 | 25 |
| I-134 | (SMAP-4DiF)-(4PEG-TBK1) | SMAP-4DiF | 4PEG-TBK1 | 1239 | 1238 | 2.36 | 25 |
| I-135 | (SMAP-3DiF)-(0PEG-TBK1) | SMAP-3DiF | 0PEG-TBK1 | 1063 | 1065 | 2.34 | 25 |
| I-136 | (JNS 1-40)-(0PEG-TBK1) | JNS 1-40 | 0PEG-TBK1 | 908 | 909 | 1.94 | 25 |
| I-137 | (JNS 1-40)-(1PEG-TBK1) | JNS 1-40 | 1PEG-TBK1 | 952 | 953 | 1.95 | 25 |
| I-138 | (JNS 1-40)-(2PEG-TBK1) | JNS 1-40 | 2PEG-TBK1 | 996 | 997 | 1.97 | 25 |
| I-139 | (JNS 1-40)-(3PEG-TBK1) | JNS 1-40 | 3PEG-TBK1 | 1040 | 1041 | 2.00 | 25 |
| I-140 | (JNS 1-40)-(4PEG-TBK1) | JNS 1-40 | 4PEG-TBK1 | 1084 | 1085 | 2.01 | 25 |

TABLE 2
Chemical Structure for Abbreviations Used in Description of Product Amide Compounds

| Abbreviation | Chemical Structure |
|---|---|
| (Ac-RVSF)- | 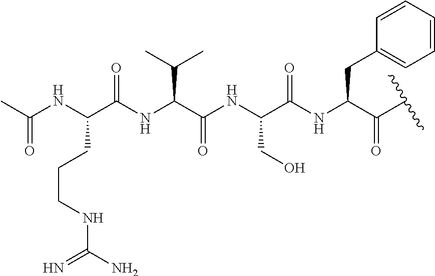 [SMILES: *C(=O)[C@H](Cc1ccccc1)NC(=O)[C@H](CO)NC(=O)[C@@H](NC(=O)[C@H](CCCNC(=N)N)NC(C)=O)C(C)C] |
| (Oct-RVSF)- | 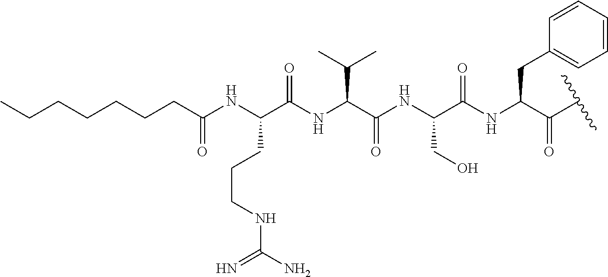 [SMILES: *C(=O)[C@H](Cc1ccccc1)NC(=O)[C@H](CO)NC(=O)[C@@H](NC(=O)[C@H](CCCNC(=N)N)NC(=O)CCCCCCC)C(C)C] |
| (H₂N-RVSF)- | 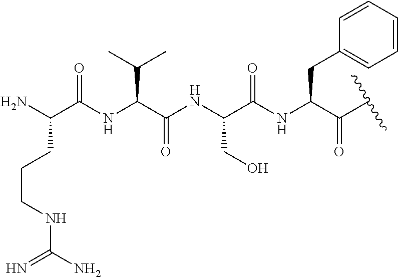 [SMILES: *C(=O)[C@H](Cc1ccccc1)NC(=O)[C@H](CO)NC(=O)[C@@H](NC(=O)[C@@H](N)CCCNC(=N)N)C(C)C] |
| (1H4)- | 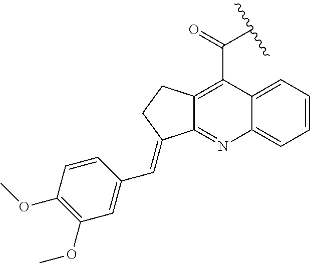 [SMILES: *C(=O)c1c2c(nc3ccccc13)/C(=C/c1ccc(OC)c(OC)c1)CC2] |
| (SMAP-Direct)- | 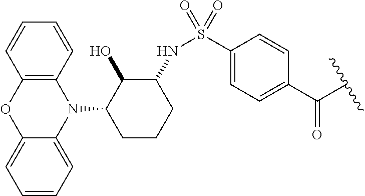 [SMILES: *C(=O)c1ccc(S(=O)(=O)N[C@@H]2CCC[C@H](N3c4ccccc4Oc4ccccc43)[C@H]2O)cc1] |
| (SMAP-4DiF)- | 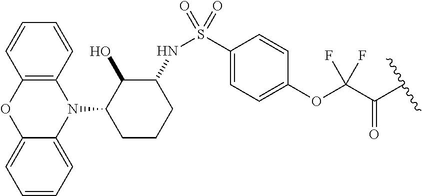 [SMILES: *C(=O)C(F)(F)Oc1ccc(S(=O)(=O)N[C@@H]2CCC[C@H](N3c4ccccc4Oc4ccccc43)[C@H]2O)cc1] |
| (SMAP-3DiF)- | 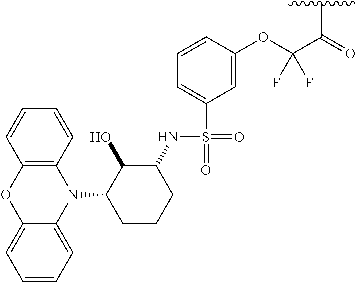 [SMILES: *C(=O)C(F)(F)Oc1cccc(S(=O)(=O)N[C@@H]2CCC[C@H](N3c4ccccc4Oc4ccccc43)[C@H]2O)c1] |
| (JNS 1-40)- | 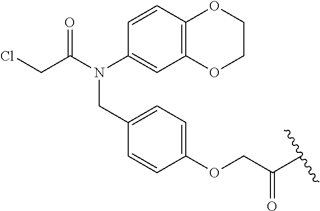 [SMILES: *C(=O)COc1ccc(CN(C(=O)CCl)c2ccc3c(c2)OCCO3)cc1] |
| -(0PEG-Chloroalkane) | 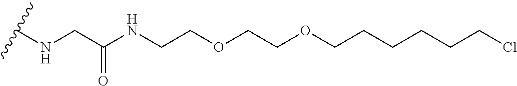 [SMILES: *NCC(=O)NCCOCCOCCCCCCCl] |
| -(1PEG-Chloroalkane) | 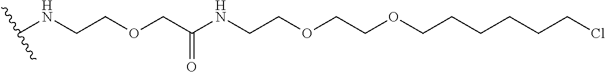 [SMILES: *NCCOCC(=O)NCCOCCOCCCCCCCl] |
| -(2PEG-Chloroalkane) | 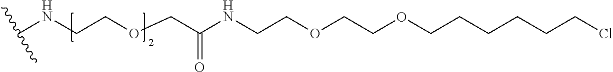 |
| -(3PEG-Chloroalkane) | 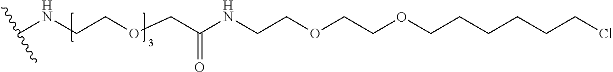 |
| -(4PEG-Chloroalkane) | 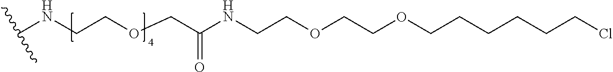 |
| -(0PEG-AKTallo) | 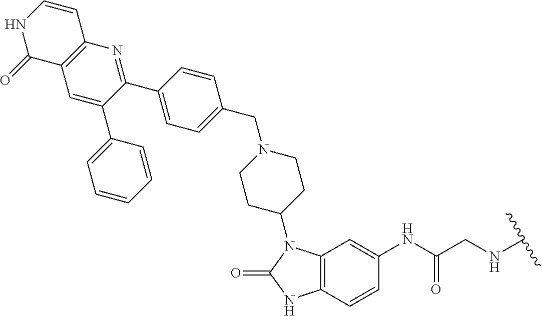 [SMILES: *NCC(=O)Nc1ccc2[nH]c(=O)n(C3CCN(Cc4ccc(-c5nc6cc[nH]c(=O)c6cc5-c5ccccc5)cc4)CC3)c2c1] |
| -(1PEG-AKTallo) | 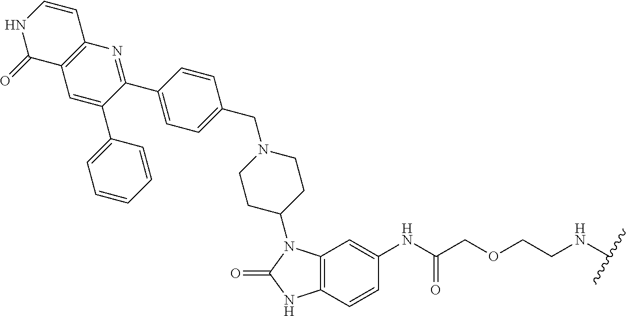 [SMILES: *NCCOCC(=O)Nc1ccc2[nH]c(=O)n(C3CCN(Cc4ccc(-c5nc6cc[nH]c(=O)c6cc5-c5ccccc5)cc4)CC3)c2c1] |
| -(2PEG-AKTallo) | 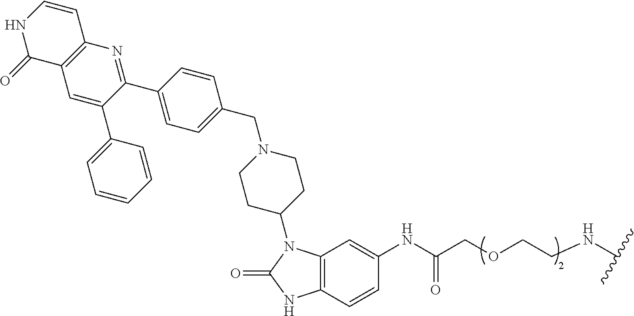 |
| -(3PEG-AKTallo) | 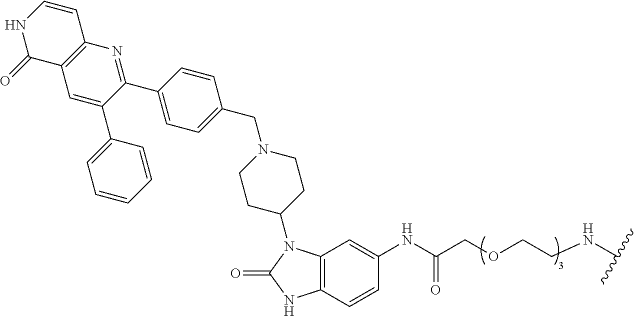 |
| -(4PEG-AKTallo) | 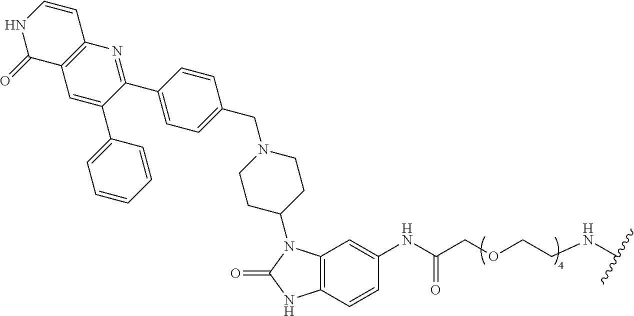 |
| -(0PEG-AKTcomp) | 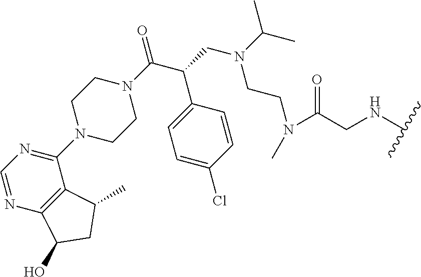 [SMILES: *NCC(=O)N(C)CCN(C[C@@H](C(=O)N1CCN(c2ncnc3c2[C@H](C)C[C@H]3O)CC1)c1ccc(Cl)cc1)C(C)C] |
| -(1PEG-AKTcomp) | 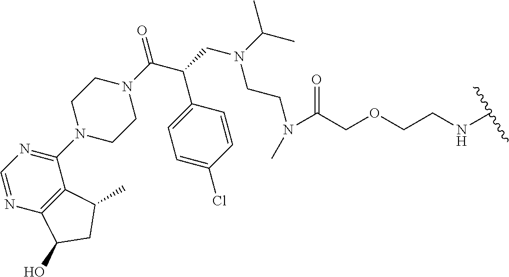 [SMILES: *NCCOCC(=O)N(C)CCN(C[C@@H](C(=O)N1CCN(c2ncnc3c2[C@H](C)C[C@H]3O)CC1)c1ccc(Cl)cc1)C(C)C] |
| -(2PEG-AKTcomp) | 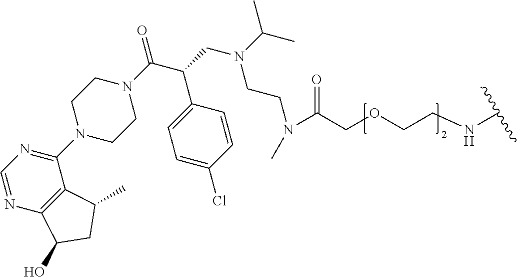 |
| -(3PEG-AKTcomp) | 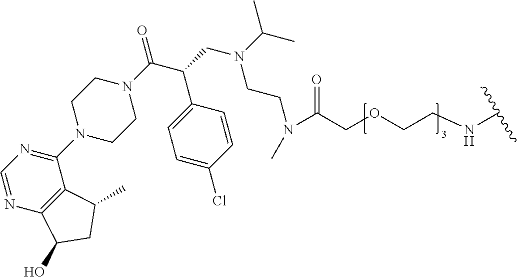 |
| -(4PEG-AKTcomp) | 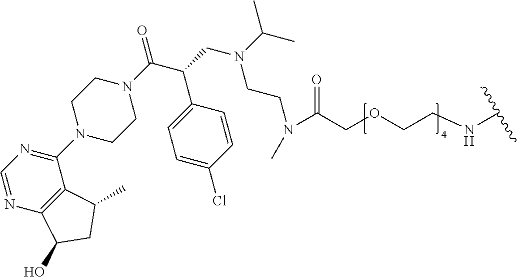 |
| -(0PEG-TBK1) | 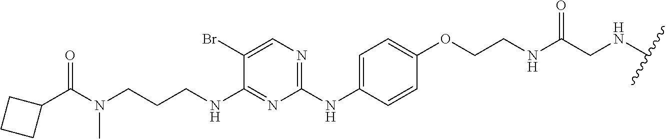 [SMILES: *NCC(=O)NCCOc1ccc(Nc2ncc(Br)c(NCCCN(C)C(=O)C3CCC3)n2)cc1] |
| -(1PEG-TBK1) | 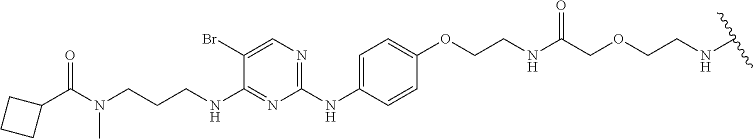 [SMILES: *NCCOCC(=O)NCCOc1ccc(Nc2ncc(Br)c(NCCCN(C)C(=O)C3CCC3)n2)cc1] |
| -(2PEG-TBK1) | 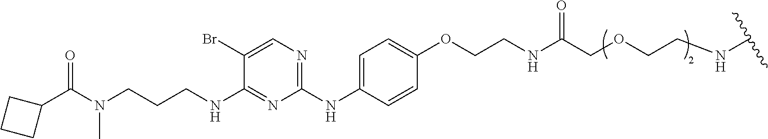 |
| -(3PEG-TBK1) | 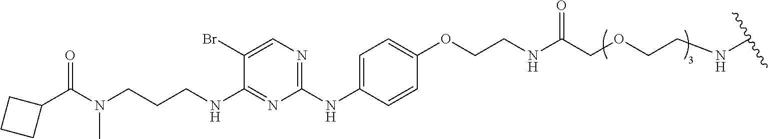 |
| -(4PEG-TBK1) | 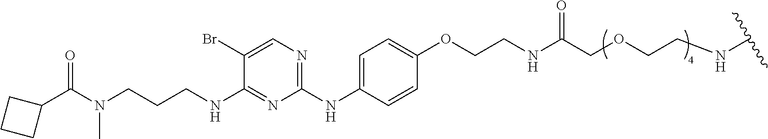 |

### Example 12: Additional Product Amide Compounds for Preparation from Ester-Containing Protein Phosphatase Ligands and Amine-Containing Linker-Target Protein Ligands

Product amide compounds in Table 3 below may be prepared from ester-containing protein phosphatase ligands and amine-containing linker-target protein ligands according to the general synthetic procedures described in Part IV of Example 11. Chemical structures for abbreviations used in the description of product amide compounds in Table 3 are provided in Table 2 above.

TABLE 3
Exemplary Compounds

| | | Protein | |
|---|---|---|---|
| | | Phosphatase | Linker-Target |
| | | Ligand | Protein Ligand |
| | | To Be | Ligand To Be |
| | | Used to | Used to |
| | | Prepare | Prepare |
| | | Product | Product |
| Compound | | Amide | Amide |
| No. | Product Amide Compound | Compound | Compound |
| I-141 | (SMAP-3DiF)- | SMAP-3DiF | 4PEG- |
| | (4PEG-Chloroalkane) | | Chloroalkane |
| I-142 | (SMAP-3DiF)-(1PEG-TBK1) | SMAP-3DiF | 1PEG-TBK1 |
| I-143 | (SMAP-3DiF)-(2PEG-TBK1) | SMAP-3DiF | 2PEG-TBK1 |
| I-144 | (SMAP-3DiF)-(3PEG-TBK1) | SMAP-3DiF | 3PEG-TBK1 |
| I-145 | (SMAP-3DiF)-(4PEG-TBK1) | SMAP-3DiF | 4PEG-TBK1 |

### Example 13: Preparation of Compound AKTallo-Succinate (Target Protein Ligand-Succinate)

The title compound was prepared according to the scheme and procedure below. Preparation of Ligand 2 is described in Angew. Chem. Int. Ed. 2015, 54, 10313-10316.

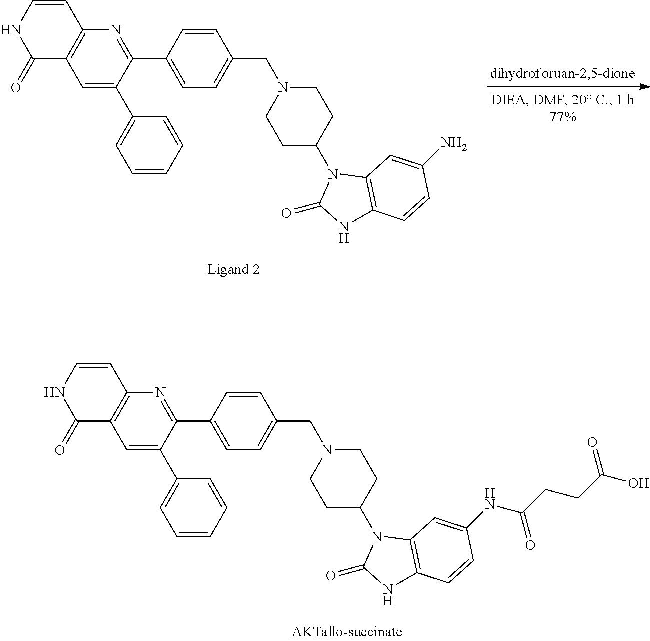 [SMILES: Nc1ccc2[nH]c(=O)n(C3CCN(Cc4ccc(-c5nc6cc[nH]c(=O)c6cc5-c5ccccc5)cc4)CC3)c2c1 ; O=C(O)CCC(=O)Nc1ccc2[nH]c(=O)n(C3CCN(Cc4ccc(-c5nc6cc[nH]c(=O)c6cc5-c5ccccc5)cc4)CC3)c2c1]

A mixture of Ligand 2 (2-[4-[[4-(6-amino-2-oxo-3H-benzimidazol-1-yl)-1-piperidyl]methyl]phenyl]-3-phenyl-6H-1,6-naphthyridin-5-one, 1.0 g, 1.7 mmol, 1.0 equiv, HCl salt), succinic anhydride (259 mg, 2.59 mmol, 1.5 equiv), DIEA (669 mg, 5.18 mmol, 0.9 mL, 3 equiv) in DMF (15 mL) was stirred at 20° C. for 1 h. The mixture was purified by prep-HPLC (column: Phenomenex luna C18 150*40 mm*15 μm; mobile phase: [water(0.1% TFA)-ACN]; B %: 10%-40%, 10 min) to afford AKTallo-succinate (4-oxo-4-[[2-oxo-3-[1-[[4-(5-oxo-3-phenyl-6H-1,6-naphthyridin-2-yl)phenyl]methyl]-4-piperidyl]-1H-benzimidazol-5-yl]amino]butanoic acid) (1.0 g, 1.3 mmol, 77% yield, TFA salt) as a yellow solid. LC-MS: MS (ES⁺): RT=0.694 min, m/z=643.1 [M+H⁺]; ¹H NMR (DMSO-d₆, 400 MHz) δ 11.59-11.57 (d, 1H, J=5.6 Hz), 10.74 (s, 1H), 9.87 (s, 1H), 8.39 (s, 1H), 8.16 (s, 1H), 7.62 (s, 1H), 7.36-7.34 (t, 1H, J=8.4 Hz), 7.32 (m, 9H), 7.26-7.24 (d, 1H, J=7.6 Hz), 6.88-6.86 (d, 1H, J=8.4 Hz), 6.68 (d, 1H, J=8.4 Hz), 4.11-4.10 (m, 1H), 3.54 (s, 2H), 2.96-2.93 (m, 2H), 2.52 (m, 4H), 2.28 (m, 2H), 2.10-2.12 (m, 2H), 1.65-1.63 (m, 2H).

### Example 14: Preparation of Compound AKTcomp-Succinate (Target Protein Ligand-Succinate)

The title compound was prepared according to the scheme and procedure below. Preparation of Compound 6 is described in Example 9, above.

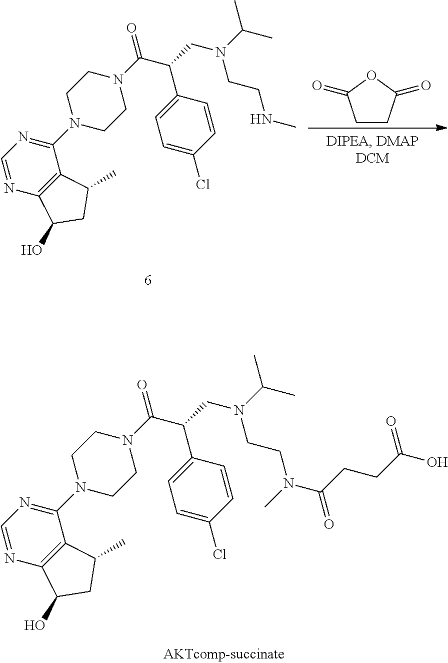 [SMILES: CNCCN(C[C@@H](C(=O)N1CCN(c2ncnc3c2[C@H](C)C[C@H]3O)CC1)c1ccc(Cl)cc1)C(C)C ; O=C1CCC(=O)O1 ; CC(C)N(CCN(C)C(=O)CCC(=O)O)C[C@@H](C(=O)N1CCN(c2ncnc3c2[C@H](C)C[C@H]3O)CC1)c1ccc(Cl)cc1]

To a solution of (S)-2-(4-chlorophenyl)-1-(4-((5R,7R)-7-hydroxy-5-methyl-6,7-dihydro-5H-cyclopenta[d]pyrimidin-4-yl)piperazin-1-yl)-3-(isopropyl(2-(methylamino)ethyl)amino)propan-1-one (Compound 6, 300 mg, 544 μmol, 1 equiv, HCl salt) and succinic anhydride (50 mg, 500 μmol, 0.92 equiv) in DCM (2 mL) were added DIPEA (223 mg, 1.72 mmol, 0.3 mL, 3.17 equiv) and DMAP (6.0 mg, 49 μmol, 0.09 equiv). The mixture was stirred at 25° C. for 3 h. The organic solvent was concentrated and the residue was purified via a Waters Xbridge 10 micron 150×50 mm C18 column with ammonia bicarbonate water/acetonitrile mobile phase to afford
AKTcomp-succinate (4-[2-[[(2S)-2-(4-chlorophenyl)-3-[4-[(5R,7R)-7-hydroxy-5-methyl-6,7-dihydro-5H-cyclopenta[d]pyrimidin-4-yl]piperazin-1-yl]-3-oxo-propyl]-isopropyl-amino]ethyl-methylamino]-4-oxo-butanoic acid) (200 mg, 60% yield) as a brown solid. ¹H NMR (400 MHz, CDCl₃): δ 8.43 (d, 1H, J=2.1 Hz), 7.37 (s, 4H), 4.83 (t, 1H, J=6.7 Hz), 4.10-4.25 (m, 1H), 2.90-3.78 (m, 14H), 2.89 (s, 3H), 2.77 (s, 1H), 2.40-2.68 (m, 6H), 1.80-2.05 (m, 2H), 1.04 (dd, 3H, J=2.2, 6.8 Hz), 0.70-1.05 (m, 6H).

### Example 15: Preparation of Compound TBK1-Succinate (Target Protein Ligand-Succinate)

The title compound was prepared according to the scheme and procedure below. Preparation of Compound 3 is described in Example 10, above.

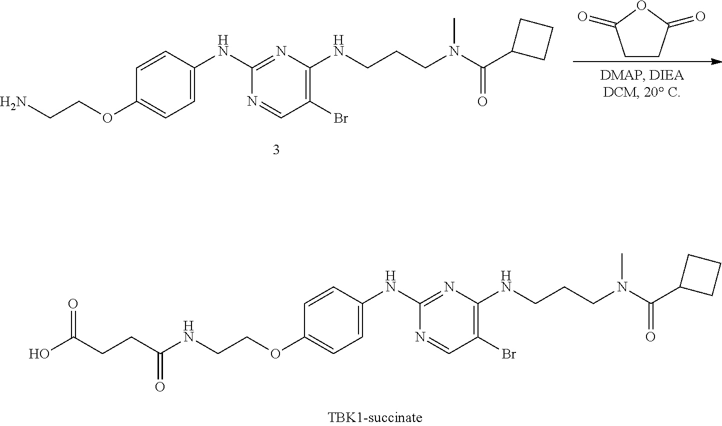

A mixture of N-(3-((2-((4-(2-aminoethoxy)phenyl)amino)-5-bromopyrimidin-4-yl)amino)propyl)-N-methylcyclobutanecarboxamide (Compound 3, 2.5 g, 4.9 mmol, 1 equiv, HCl salt), DIEA (1.8 g, 14 mmol, 2.4 mL, 2.8 equiv), succinic anhydride (880 mg, 8.79 mmol, 1.8 equiv) and DMAP (535 mg, 4.38 mmol, 0.9 equiv) in DCM (5 mL) was stirred at 20° C. for 16 h. The reaction was quenched with 1 M HCl, and the pH was adjusted to 3. The solvent was removed in vacuo, and the residue was purified by a 10 micron Phenomenex Synergi Max-RP 250×50 mm×10 um; mobile phase: [water(0.225% FA)-ACN]; B %: 20ACN % −50ACN %, 20 min to afford compound TBK1-succinate. ¹H NMR (DMSO-d₆, 400 MHz): δ 12.05 (br s, 1H), 9.03 (s, 1H), 8.09 (t, 1H, J=5.5 Hz), 7.94-8.01 (m, 1H), 7.54-7.64 (m, 2H), 6.93-7.05 (m, 1H), 6.87 (dd, 2H, J=9.0, 1.9 Hz), 3.92 (t, 2H, J=5.7 Hz), 3.30-3.48 (m, 3H), 3.18-3.24 (m, 1H), 2.84 (s, 3H), 2.40-2.45 (m, 2H), 2.32-2.38 (m, 2H), 2.07-2.18 (m, 3H), 1.65-1.98 (m, 4H).

### Example 16: Preparation of Compound Chloroalkane-Succinate (Target Protein Ligand-Succinate)

The title compound was prepared according to the scheme and procedure below.

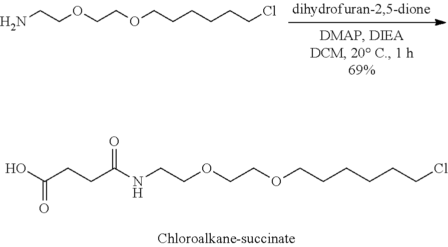 [SMILES: NCCOCCOCCCCCCCl ; O=C(O)CCC(=O)NCCOCCOCCCCCCCl]

To a mixture of 2-[2-(6-chlorohexoxy)ethoxy]ethanamine (600 mg, 2.68 mmol, 1.0 equiv, HCl salt), DIEA (970 mg, 7.51 mmol, 1.3 mL, 2.8 equiv) and DMAP (294 mg, 2.41 mmol, 0.9 equiv) in DCM (6 mL) was added succinic anhydride (483 mg, 4.83 mmol, 1.8 equiv). The reaction was stirred at 20° C. for 1 h. The reaction mixture was quenched with 1 M HCl to pH=3, and then extracted with CH₂Cl₂ (2×50 mL). The organic layers were combined, washed with brine (20 mL), dried over Na₂SO₄, filtered, and concentrated. The resulting residue was purified by prep-HPLC (10 micron Waters Xbridge 150×25 mm column with FA-modified water/acetonitrile mobile phase). Compound Chloroalkane-succinate (4-[2-[2-(6-chlorohexoxy)ethoxy]ethylamino]-4-oxo-butanoic acid) (0.6 g, 1.85 mmol, 69.10% yield) was obtained as a colorless oil. ¹H NMR (400 MHz, CDCl₃): δ 6.44 (brs, 1H), 3.61-3.65 (s, 4H), 3.51-3.57 (m, 5H), 3.44-3.51 (m, 3H), 2.65-2.72 (m, 2H), 2.51-2.56 (m, 2H), 1.74-1.82 (m, 2H), 1.64 (m, 2H), 1.34-1.52 (m, 4H).

### Example 17: Preparation of Compounds (0-2)PEG-RVSF-NMe and (0-2)PEG-RVSF-NOct (Linker-Protein Phosphatase Ligand)

The title compounds were prepared according to the general synthetic procedures below, as described and depicted in the scheme. Preparation of Compound 1 in the scheme below is described in Example 2, above.

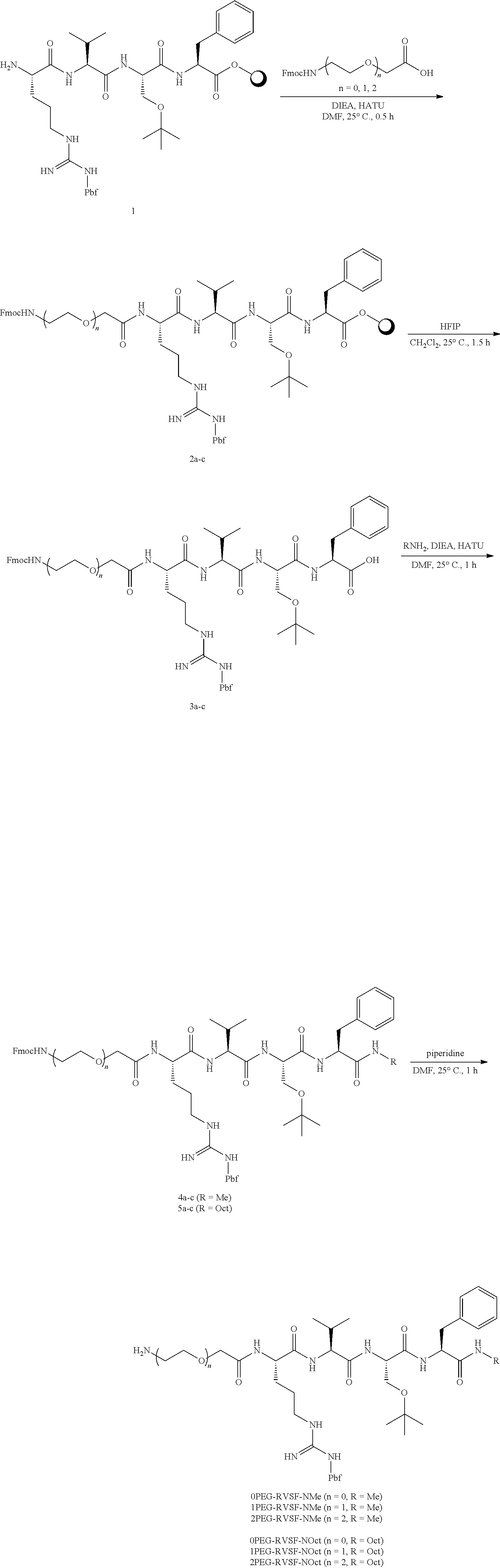

### Part I-Preparation of Compounds 3a-c

To resin-bound compound 1 was added a DMF (20 mL) solution of FmocNH-PEG-COOH (15 mmol, 1.5 equiv), DIEA (30 mmol, 4.0 mL, 3.0 equiv), and HATU (11 mmol, 4.2 g, 1.5 equiv), and the solution was bubbled with N₂ for 0.5 h. The reaction mixture was filtered, and the resin was washed with DMF (3×40 mL), MeOH (3×40 mL) followed by CH₂Cl₂ (3×40 mL) to afford compounds 2a-c on the resin. 1,1,1,3,3,3-hexafluoropropan-2-ol (11 g, 67 mmol, 3.0 mL, 10 equiv) in CH₂Cl₂ (30 mL) was added to the resin and the solution was bubbled with N₂ at 25° C. for 1.5 h. The reaction mixture was filtered, and the filtrate was concentrated to afford compounds 3a-c as a white solid, which was used directly in the next step without further purification.

### Physical Characterization Data for Compounds 3a-c:

Compound 3a ((8S,11S,14S,17S)-17-benzyl-14-(tert-butoxymethyl)-1-(9H-fluoren-9-yl)-11-isopropyl-3,6,9,12,15-pentaoxo-8-(3-(3-((2,2,4,6,7-pentamethyl-2,3-dihydrobenzofuran-5-yl)sulfonyl)guanidino)propyl)-2-oxa-4,7,10,13,16-pentaazaoctadecan-18-oic acid) LC-MS: MS (ES⁺): RT=1.024 min, m/z=1095.3 [M+H⁺].
Compound 3b ((11 S,14S,17S,20S)-20-benzyl-17-(tert-butoxymethyl)-1-(9H-fluoren-9-yl)-14-isopropyl-3,9,12,15,18-pentaoxo-11-(3-(3-((2,2,4,6,7-pentamethyl-2,3-dihydrobenzofuran-5-yl)sulfonyl)guanidino)propyl)-2,7-dioxa-4,10,13,16,19-pentaazahenicosan-21-oic acid) LC-MS: MS (ES⁺): RT=0.975 min, m/z=1139.6 [M+H⁺].
Compound 3c ((14S,17S,20S,23 S)-23-benzyl-20-(tert-butoxymethyl)-1-(9H-fluoren-9-yl)-17-isopropyl-3,12,15,18,21-pentaoxo-14-(3-(3-((2,2,4,6,7-pentamethyl-2,3-dihydrobenzofuran-5-yl)sulfonyl)guanidino)propyl)-2,7,10-trioxa-4,13,16,19,22-pentaazatetracosan-24-oic acid) LC-MS: MS (ES⁺): RT=0.980 min, m/z=1183.7 [M+H⁺].

### Part II-General Procedure for the Synthesis of Compounds (0-2)PEG-RVSF-NMe

To a solution of compound 3a-c (2.0 mmol, 1.0 equiv), methylamine (4.0 mmol, 2.0 equiv) in DMF (10 mL) was added HATU (4.0 mmol, 1.5 g, 2.0 equiv) and DIEA (6.0 mmol, 3.0 equiv). The mixture was stirred at 25° C. for 1 h. Piperidine (20 mmol, 2.0 mL, 10 equiv) was added, and the reaction mixture was stirred at 25° C. for another 1 h. The reaction mixture was neutralized by HOAc, and then purified by prep-HPLC on a Waters Xbridge BEH C18 250*50 mm*10 μm column with acetonitrile and ammonia hydroxide modified water as a mobile phase to afford compounds (0-2)PEG-RVSF-NMe as a white solid.

### Physical Characterization Data for Compounds (0-2)PEG-RVSF-NMe:

Compound 0PEG-RVSF-NMe ((S)-2-(2-aminoacetamido)-N-((S)-1-(((S)-3-(tert-butoxy)-1-(((S)-1-(methylamino)-1-oxo-3-phenylpropan-2-yl)amino)-1-oxopropan-2-yl)amino)-3-methyl-1-oxobutan-2-yl)-5-(3-((2,2,4,6,7-pentamethyl-2,3-dihydrobenzofuran-5-yl)sulfonyl)guanidino)pentanamide) LC-MS: MS (ES⁺): RT=0.797 min, m/z=886.2 [M+H⁺]; ¹H NMR (CD₃OD, 400 MHz) δ 7.1-7.3 (m, 5H), 4.5-4.7 (m, 2H), 4.47 (dd, 1H, J=5.7, 8.2 Hz), 4.40 (t, 1H, J=5.7 Hz), 4.1-4.2 (m, 1H), 3.66 (s, 2H), 3.6-3.6 (m, 1H), 3.51 (dd, 1H, J=6.4, 9.1 Hz), 3.0-3.3 (m, 3H), 2.99 (s, 2H), 2.9-3.0 (m, 1H), 2.69 (d, 1H, J=3.8 Hz), 2.66 (s, 2H), 2.57 (s, 3H), 2.51 (s, 3H), 2.0-2.1 (m, 4H), 1.8-1.9 (m, 1H), 1.6-1.7 (m, 1H), 1.5-1.6 (m, 2H), 1.45 (s, 6H), 1.14 (s, 9H), 0.9-1.0 (m, 6H).
Compound 1PEG-RVSF-NMe ((S)-2-(2-(2-aminoethoxy)acetamido)-N-((S)-1-(((S)-3-(tert-butoxy)-1-(((S)-1-(methylamino)-1-oxo-3-phenylpropan-2-yl)amino)-1-oxopropan-2-yl)amino)-3-methyl-1-oxobutan-2-yl)-5-(3-((2,2,4,6,7-pentamethyl-2,3-dihydrobenzofuran-5-yl)sulfonyl)guanidino)pentanamide) LC-MS: MS (ES⁺): RT=0.793 min, m/z=930.3 [M+H⁺]; ¹H NMR (CD₃OD, 400 MHz) δ 7.1-7.3 (m, 5H), 4.58 (m, 1H), 4.52 (dd, 1H, J=5.9, 8.2 Hz), 4.41 (t, 1H, J=5.7 Hz), 4.21 (d, 1H, J=6.8 Hz), 4.0-4.2 (m, 2H), 3.7-3.8 (m, 2H), 3.6-3.6 (m, 1H), 3.5-3.5 (m, 1H), 3.0-3.3 (m, 5H), 2.99 (s, 2H), 2.9-3.0 (m, 1H), 2.6-2.7 (s, 3H), 2.57 (s, 3H), 2.51 (s, 3H), 2.0-2.1 (m, 4H), 1.8-1.9 (m, 1H), 1.6-1.8 (m, 1H), 1.5-1.6 (m, 2H), 1.45 (s, 6H), 1.14 (s, 9H), 0.9-1.0 (m, 6H).
Compound 2PEG-RVSF-NMe ((S)-2-(2-(2-(2-aminoethoxy)ethoxy)acetamido)-N-((S)-1-(((S)-3-(tert-butoxy)-1-(((S)-1-(methylamino)-1-oxo-3-phenylpropan-2-yl)amino)-1-oxopropan-2-yl)amino)-3-methyl-1-oxobutan-2-yl)-5-(3-((2,2,4,6,7-pentamethyl-2,3-dihydrobenzofuran-5-yl)sulfonyl)guanidino)pentanamide) LC-MS: MS (ES⁺): RT=0.795 min, m/z=974.4 [M+H⁺]; ¹H NMR (CD₃OD, 400 MHz) δ 7.1-7.3 (m, 5H), 4.58 (m, 2H), 4.39 (t, 1H, J=5.6 Hz), 4.1-4.2 (m, 1H), 4.0-4.1 (m, 2H), 3.7-3.8 (m, 6H), 3.6-3.6 (m, 1H), 3.5-3.6 (m, 1H), 3.0-3.3 (m, 5H), 2.99 (s, 2H), 2.9-3.0 (m, 1H), 2.66 (s, 3H), 2.57 (s, 3H), 2.51 (s, 3H), 2.0-2.2 (m, 4H), 1.8-1.9 (m, 1H), 1.66 (m, 1H), 1.5-1.6 (m, 2H), 1.45 (s, 6H), 1.14 (s, 9H), 0.9-1.0 (m, 6H).

### Part III-General Procedure for the Synthesis of Compounds (0-2)PEG-RVSF-NOct

To a solution of compound 3a-c (2.0 mmol, 1.0 equiv), octylamine (4.0 mmol, 2.0 equiv) in DMF (10 mL) was added HATU (4.0 mmol, 1.5 g, 2.0 equiv) and DIEA (6.0 mmol, 3.0 equiv). The mixture was stirred at 25° C. for 1 h. Piperidine (20 mmol, 2.0 mL, 10 equiv) was added, and the reaction mixture was stirred at 25° C. for another 1 h. The reaction mixture was neutralized by HOAc, and then purified by prep-HPLC on a Waters Xbridge BEH C18 250*50 mm*10 μm column with acetonitrile and ammonia hydroxide modified water as a mobile phase to afford compounds (0-2)PEG-RVSF-NOct as a white solid.

### Physical Characterization Data for Compounds (0-2)PEG-RVSF-NOct:

Compound 0PEG-RVSF-NOct ((S)-2-(2-aminoacetamido)-N-((S)-1-(((S)-3-(tert-butoxy)-1-(((S)-1-(octylamino)-1-oxo-3-phenylpropan-2-yl)amino)-1-oxopropan-2-yl)amino)-3-methyl-1-oxobutan-2-yl)-5-(3-((2,2,4,6,7-pentamethyl-2,3-dihydrobenzofuran-5-yl)sulfonyl)guanidino)pentanamide) LC-MS: MS (ES⁺): RT=0.901 min, m/z=984.4 [M+H⁺]; ¹H NMR (CD₃OD, 400 MHz) δ 7.1-7.3 (m, 5H), 4.5-4.7 (m, 2H), 4.3-4.5 (m, 2H), 4.20 (d, 1H, J=6.8 Hz), 3.6-3.7 (m, 3H), 3.5-3.6 (m, 1H), 2.9-3.2 (m, 9H), 2.57 (s, 3H), 2.51 (s, 3H), 2.07 (s, 3H), 1.8-1.9 (m, 1H), 1.6-1.7 (m, 1H), 1.5-1.6 (m, 2H), 1.45 (s, 6H), 1.2-1.4 (m, 10H), 1.14 (s, 9H), 0.9-1.0 (m, 9H).
Compound 1PEG-RVSF-NOct ((S)-2-(2-(2-aminoethoxy)acetamido)-N-((S)-1-(((S)-3-(tert-butoxy)-1-(((S)-1-(octylamino)-1-oxo-3-phenylpropan-2-yl)amino)-1-oxopropan-2-yl)amino)-3-methyl-1-oxobutan-2-yl)-5-(3-((2,2,4,6,7-pentamethyl-2,3-dihydrobenzofuran-5-yl)sulfonyl)guanidino)pentanamide) LC-MS: MS (ES⁺): RT=0.906 min, m/z=1028.4 [M+H⁺]; ¹H NMR (CD₃OD, 400 MHz) δ 7.1-7.3 (m, 5H), 4.5-4.7 (m, 3H), 4.41 (q, 1H, J=4.9 Hz), 4.1-4.3 (m, 1H), 4.10 (d, 1H, J=7.1 Hz), 4.05 (d, 1H, J=3.2 Hz), 3.7-3.8 (m, 4H), 3.6-3.6 (m, 1H), 3.53 (m, 1H), 3.1-3.2 (m, 5H), 2.9-3.1 (m, 5H), 2.57 (s, 3H), 2.51 (s, 3H), 2.0-2.2 (m, 4H), 1.8-1.9 (m, 1H), 1.68 (td, 1H, J=7.2, 14.5 Hz), 1.55 (m, 2H), 1.45 (s, 6H), 1.2-1.4 (m, 10H), 1.14 (s, 9H), 0.8-1.0 (m, 9H).
Compound 2PEG-RVSF-NOct ((S)-2-(2-(2-(2-aminoethoxy)ethoxy)acetamido)-N-((S)-1-(((S)-3-(tert-butoxy)-1-(((S)-1-(octylamino)-1-oxo-3-phenylpropan-2-yl)amino)-1-oxopropan-2-yl)amino)-3-methyl-1-oxobutan-2-yl)-5-(3-((2,2,4,6,7-pentamethyl-2,3-dihydrobenzofuran-5-yl)sulfonyl)guanidino)pentanamide) LC-MS: MS (ES⁺): RT=0.906 min, m/z=1072.3 [M+H⁺]; ¹H NMR (CD₃OD, 400 MHz) δ 7.1-7.3 (m, 5H), 4.59 (m, 2H), 4.40 (m, 1H), 4.19 (d, 1H, J=6.7 Hz), 4.0-4.1 (m, 2H), 3.7-3.8 (m, 6H), 3.5-3.6 (m, 3H), 2.9-3.3 (m, 11H), 2.57 (s, 3H), 2.51 (s, 3H), 2.0-2.2 (m, 4H), 1.8-1.9 (m, 1H), 1.6-1.7 (m, 1H), 1.5-1.6 (m, 2H), 1.45 (s, 6H), 1.2-1.4 (m, 10OH), 1.14 (s, 9H), 0.8-1.0 (m, 9H).

### Example 18: Preparation of Compounds (3-4)PEG-RVSF-NMe and (3-4)PEG-RVSF-NOct (Linker-Protein Phosphatase Ligand)

The title compounds were prepared according to the general synthetic procedures below, as described and depicted in the scheme. Preparation of H₂N-RVSF in the scheme below is described in Example 2, above.

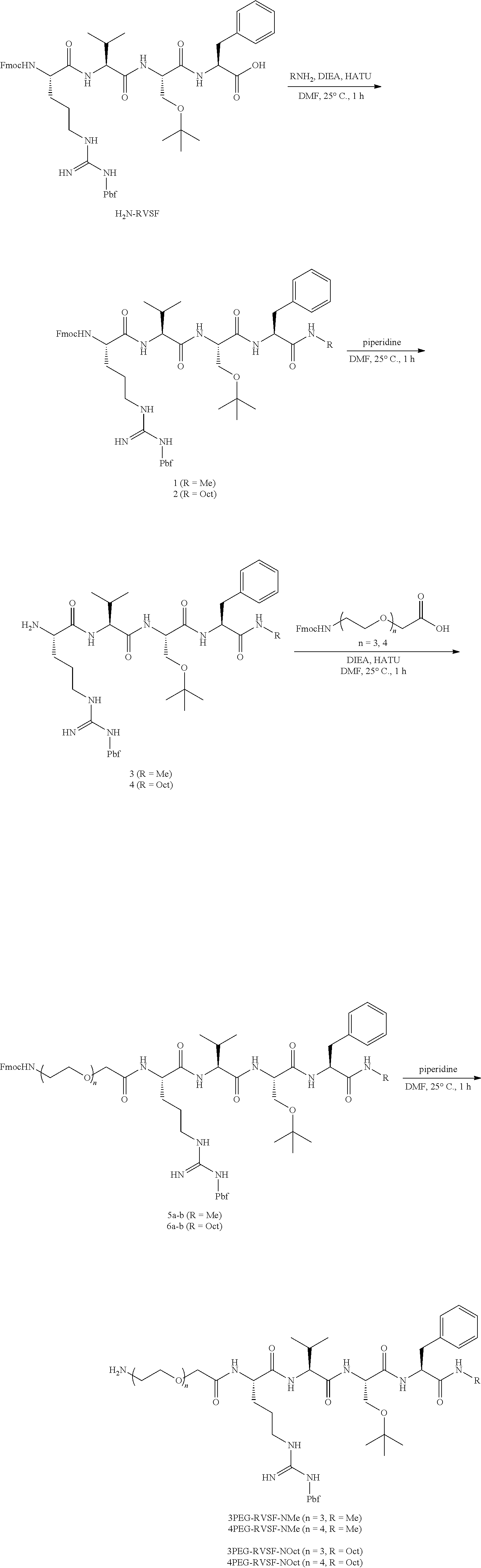

### Part I-General Procedure for the Synthesis of Compound 3 and 4

To a solution of H₂N-RVSF (2.3 g, 2.0 mmol, 1.0 equiv), RNH₂ (4.0 mmol, 2.0 equiv) (RNH₂=methylamine and octylamine) in DMF (8 mL) was added DIEA (1.0 g, 8.1 mmol, 1.4 mL, 4.0 equiv) and HATU (1.6 g, 4.0 mmol, 2.0 equiv). The mixture was stirred at 25° C. for 1 h. Piperidine (1.7 g, 20 mmol, 2.0 mL, 10 equiv) was added and stirred at 25° C. for 1 h. The reaction mixture was neutralized by HOAc, and then it was purified by prep-HPLC on a Phenomenex Synergi Max-RP 250*50 mm*10 μm column with actonitrile and TFA-modified water as mobile phase to afford compound 3 or 4 (TFA salt) as a white solid.

### Physical Characterization Data for Compounds 3 and 4:

Compound 3 (R=Me, (S)-2-amino-N-((S)-1-(((S)-3-(tert-butoxy)-1-(((S)-1-(methylamino)-1-oxo-3-phenylpropan-2-yl)amino)-1-oxopropan-2-yl)amino)-3-methyl-1-oxobutan-2-yl)-5-(3-((2,2,4,6,7-pentamethyl-2,3-dihydrobenzofuran-5-yl)sulfonyl)guanidino)pentanamide) LC-MS: MS (ES⁺): RT=0.857 min, m/z=829.5 [M+H⁺].
Compound 4 (R=Oct, (S)-2-amino-N-((S)-1-(((S)-3-(tert-butoxy)-1-(((S)-1-(octylamino)-1-oxo-3-phenylpropan-2-yl)amino)-1-oxopropan-2-yl)amino)-3-methyl-1-oxobutan-2-yl)-5-(3-((2,2,4,6,7-pentamethyl-2,3-dihydrobenzofuran-5-yl)sulfonyl)guanidino)pentanamide) LC-MS: MS (ES⁺): RT=0.984 min, m/z=927.6 [M+H⁺].

### Part II-General Procedure for the Synthesis of Compounds (3-4)PEG-RVSF-NMe and (3-4)PEG-RVSF-NOct

To a solution of compound 3 or 4 (2.2 mmol, 1.0 equiv), FmocNH-PEG-COOH (3.3 mmol, 1.5 equiv) in DMF (10 mL) was added DIEA (1.1 g, 8.6 mmol, 1.5 mL, 4.0 equiv) and HATU (4.3 mmol, 2.0 equiv). The mixture was stirred at 25° C. for 1 h. Piperidine (1.8 g, 22 mmol, 2.1 mL, 10.0 equiv) was added and the mixture was stirred at 25° C. for another 1 h. The reaction mixture was neutralized by HOAc, and then it was purified by prep-HPLC on a Phenomenex Synergi Max-RP 250*50 mm*10 m column with actonitrile and TFA modified water as mobile phase to afford compound 3-4-PEG-RVSF-NMe (TFA salt) as a white solid.

### Physical Characterization Data for Compounds (3-4)PEG-RVSF-NMe and (3-4)PEG-RVSF-NOct:

Compound 3PEG-RVSF-NMe ((S)-2-(2-(2-(2-(2-aminoethoxy)ethoxy)ethoxy)acetamido)-N-((S)-1-(((S)-3-(tert-butoxy)-1-(((S)-1-(methylamino)-1-oxo-3-phenylpropan-2-yl)amino)-1-oxopropan-2-yl)amino)-3-methyl-1-oxobutan-2-yl)-5-(3-((2,2,4,6,7-pentamethyl-2,3-dihydrobenzofuran-5-yl)sulfonyl)guanidino)pentanamide) LC-MS: MS (ES⁺): RT=0.799 min, m/z=1018.3 [M+H⁺]; ¹H NMR (CD₃OD, 400 MHz) δ 7.1-7.3 (m, 5H), 4.5-4.7 (m, 3H), 4.39 (t, 1H, J=5.6 Hz), 4.17 (d, 1H, J=6.8 Hz), 4.05 (s, 2H), 3.7-3.8 (m, 10H), 3.6-3.6 (m, 1H), 3.5-3.6 (m, 1H), 3.0-3.2 (m, 5H), 2.9-3.0 (m, 3H), 2.66 (s, 3H), 2.57 (s, 3H), 2.51 (s, 3H), 2.0-2.2 (m, 4H), 1.8-1.9 (m, 2H), 1.6-1.7 (m, 1H), 1.55 (m, 2H), 1.45 (s, 6H), 1.14 (s, 9H), 0.94 (m, 6H).
Compound 4PEG-RVSF-NMe (14-amino-N-((4S,7S,10OS,13S)-4-benzyl-7-(tert-butoxymethyl)-18-imino-10-isopropyl-3,6,9,12-tetraoxo-18-(2,2,4,6,7-pentamethyl-2,3-dihydrobenzofuran-5-sulfonamido)-2,5,8,11,17-pentaazaoctadecan-13-yl)-3,6,9,12-tetraoxatetradecan-1-amide) LC-MS: MS (ES⁺): RT=0.798 min, m/z=1062.4 [M+H⁺]; ¹H NMR (CD₃OD, 400 MHz) δ 7.1-7.3 (m, 5H), 4.4-4.6 (m, 3H), 4.39 (t, 1H, J=5.6 Hz), 4.18 (d, 1H, J=6.8 Hz), 4.07 (s, 2H), 3.83 (d, 1H, J=5.5 Hz), 3.7-3.7 (m, 13H), 3.51 (m, 2H), 3.1-3.2 (m, 4H), 2.9-3.0 (m, 4H), 2.6-2.7 (s, 3H), 2.57 (s, 3H), 2.51 (s, 3H), 2.0-2.2 (m, 4H), 1.8-1.9 (m, 1H), 1.7-1.7 (m, 1H), 1.5-1.6 (m, 2H), 1.45 (s, 6H), 1.14 (s, 9H), 0.93 (m, 6H).
Compound 3PEG-RVSF-NOct ((S)-2-(2-(2-(2-(2-aminoethoxy)ethoxy)ethoxy)acetamido)-N-((S)-1-(((S)-3-(tert-butoxy)-1-(((S)-1-(octylamino)-1-oxo-3-phenylpropan-2-yl)amino)-1-oxopropan-2-yl)amino)-3-methyl-1-oxobutan-2-yl)-5-(3-((2,2,4,6,7-pentamethyl-2,3-dihydrobenzofuran-5-yl)sulfonyl)guanidino)pentanamide) LC-MS: MS (ES⁺): RT=0.912 min, m/z=1116.4 [M+H⁺]; ¹H NMR (CD₃OD, 400 MHz) δ 7.1-7.3 (m, 5H), 4.5-4.7 (m, 2H), 4.40 (t, 1H, J=5.4 Hz), 4.17 (m, 1H), 4.05 (s, 2H), 3.82 (d, 1H, J=5.5 Hz), 3.7-3.7 (m, 9H), 3.60 (m, 1H), 3.5-3.6 (m, 1H), 3.4-3.5 (m, 1H), 2.9-3.2 (m, 10H), 2.57 (s, 2H), 2.51 (s, 3H), 2.0-2.2 (m, 4H), 1.8-1.9 (m, 2H), 1.7-1.7 (m, 1H), 1.55 (m, 2H), 1.44 (s, 6H), 1.3-1.4 (m, 10H), 1.1-1.2 (m, 9H), 0.8-1.0 (m, 9H).
Compound 4PEG-RVSF-NOct (14-amino-N-((6S,9S,12S,15S)-15-benzyl-12-(tert-butoxymethyl)-1-imino-9-isopropyl-7,10,3,16-tetraoxo-1-(2,2,4,6,7-pentamethyl-2,3-dihydrobenzofuran-5-sulfonamido)-2,8,11,14,17-pentaazapentacosan-6-yl)-3,6,9,12-tetraoxatetradecan-1-amide) LC-MS: MS (ES⁺): RT=0.905 min, m/z=1160.4 [M+H⁺]; ¹H NMR (CD₃OD, 400 MHz) δ 7.1-7.3 (m, 5H), 4.60 (m, 1H), 4.51 (m, 1H), 4.40 (m, 1H), 4.19 (m, 1H), 4.08 (s, 2H), 3.6-3.8 (m, 15H), 3.5-3.6 (m, 1H), 3.3-3.3 (m, 1H), 2.9-3.2 (m, 10H), 2.57 (s, 3H), 2.51 (s, 3H), 2.0-2.2 (m, 4H), 1.8-1.9 (m, 1H), 1.6-1.7 (m, 1H), 1.56 (m, 2H, J=6.8 Hz), 1.45 (s, 6H), 1.2-1.4 (m, 12H), 1.14 (s, 9H), 0.8-1.0 (m, 9H).

### Example 19: Preparation of Compounds (0-4)PEG-RVSF-OH (Linker-Protein Phosphatase Ligand)

The title compounds were prepared according to the general synthetic procedures below, as described and depicted in the scheme. Preparation of Compound H₂N-RVSF in the scheme below is described in Example 2, above.

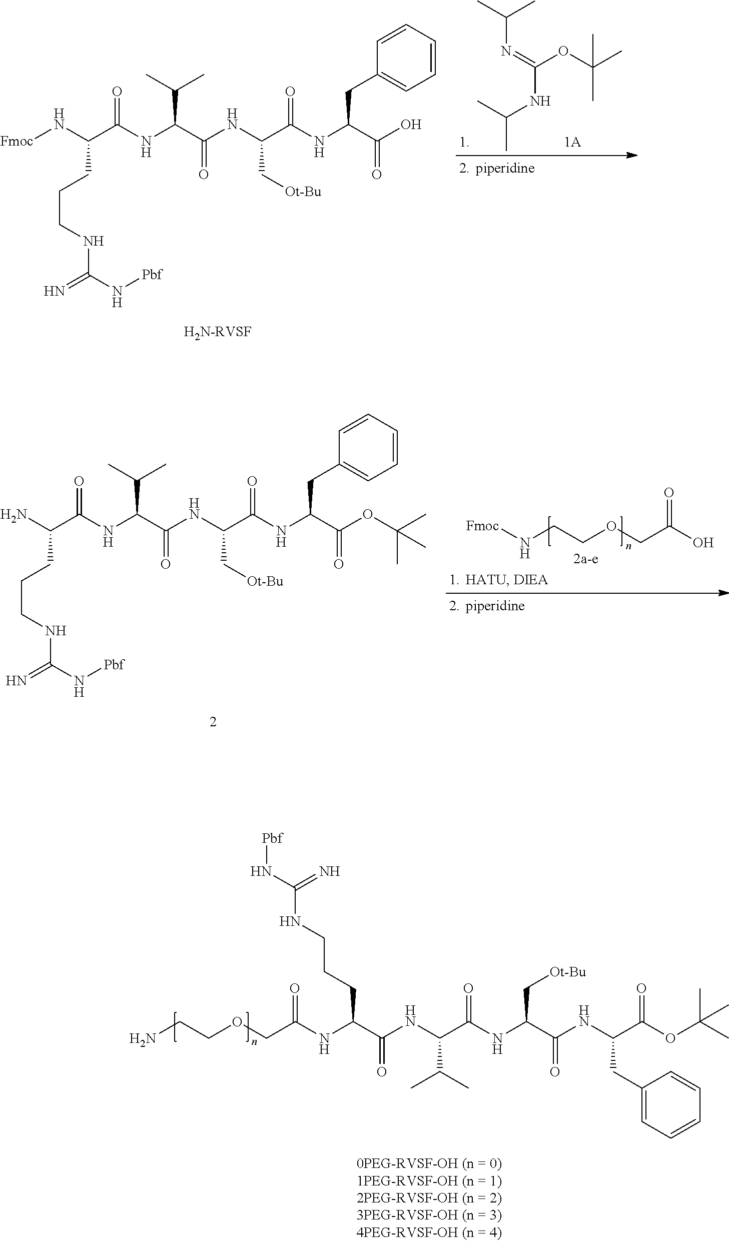

### Part I-Preparation of Compound 2

A mixture of compound H₂N-RVSF (12 g, 12 mmol, 1.0 equiv) and compound 1A (4.6 g, 23 mmol, 2.0 equiv) in DCM (150 mL) was stirred at 25° C. for 24 h, and then piperidine (8.6 g, 101 mmol, 8.8 equiv) was added, and the resulting mixture was stirred at 25° C. for another 0.5 h. The organic solvent was removed and the residue was purified on a 10 micron Phenomenex luna 250×50 mm column with HCl modified water/acetonitrile mobile phase to afford compound 2. LC-MS: MS (ES⁺): RT=0.784 min, m/z=872.3 [M+H⁺].

### Part II-Preparation of Compounds (0-4)PEG-RVSF-OH

To a solution of compound 2 (880 μmol, 1.0 equiv, HCl salt), compound 2a-e (n=0,1,2,3,4) (968 μmol, 1.1 equiv) and DIEA (1.32 mmol, 1.5 equiv) in DMF (10 mL) was added HATU (1.06 mmol, 1.2 equiv) at 0° C. The mixture was stirred at 25° C. for 12 h, and then piperidine (8.10 mmol, 9.2 equiv) was added. The mixture was stirred at 25° C. for 0.5 h. The solvent was removed, and then the residue was purified on a 15 micron Phenomenex luna 150×40 mm column with TFA modified water/acetonitrile mobile phase to afford compound (0-4)PEG-RVSF-OH.

### Physical Characterization Data for Compounds (0-4)PEG-RVSF-OH:

Compound 0PEG-RVSF-OH ((6S,9S,12S,15 S)-tert-butyl6-(2-aminoacetamido)-15-benzyl-12-(tert-butoxymethyl)-1-imino-9-isopropyl-7,10,13-trioxo-1-(2,2,4,6,7-pentamethyl-2,3-dihydrobenzofuran-5-sulfonamido)-2,8,11,14-tetraazahexadecan-16-oate)¹HNMR (400 MHz, CD₃OD): δ 7.12-7.36 (m, 5H), 4.44-4.63 (m, 3H), 4.20-4.30 (m, 1H), 3.69-3.82 (m, 2H), 3.55-3.66 (m, 2H), 3.12-3.23 (m, 2H), 2.97-3.10 (m, 4H), 2.46-2.65 (m, 6H), 2.03-2.20 (m, 4H), 1.79-1.91 (m, 1H), 1.66-1.74 (m, 1H), 1.57-1.64 (m, 2H), 1.47 (s, 6H), 1.31-1.43 (m, 9H), 1.12-1.24 (m, 9H), 0.96 (dd, 6H, J=6.8, 3.5 Hz). LC-MS: MS (ES⁺): RT=1.399 min, m/z=929.3 [M+H⁺].
Compound 1PEG-RVSF-OH ((2S,5S,8S,11 S)-tert-butyl 17-amino-2-benzyl-5-(tert-butoxymethyl)-8-isopropyl-4,7,10,13-tetraoxo-11-(3-(3-((2,2,4,6,7-pentamethyl-2,3-dihydrobenzofuran-5-yl)sulfonyl)guanidino)propyl)-15-oxa-3,6,9,12-tetraazaheptadecan-1-oate) ¹HNMR (400 MHz, CD₃OD): δ 7.16-7.34 (m, 5H), 4.45-4.65 (m, 3H), 4.26 (d, 1H, J=6.9 Hz), 4.06-4.18 (m, 2H), 3.71-3.84 (m, 2H), 3.55-3.65 (m, 2H), 3.13-3.27 (m, 4H), 2.97-3.10 (m, 4H), 2.45-2.64 (m, 6H), 2.04-2.18 (m, 4H), 1.82-1.96 (m, 1H), 1.69-1.77 (m, 1H), 1.55-1.64 (m, 2H), 1.47 (s, 6H), 1.39 (s, 9H), 1.12-1.22 (m, 9H), 0.96 (dd, 6H, J=6.8, 3.0 Hz). LC-MS: MS (ES⁺): RT=1.395 min, m/z=973.3 [M+H⁺].
Compound 2PEG-RVSF-OH ((2S,5S,8S,11 S)-tert-butyl20-amino-2-benzyl-5-(tert-butoxymethyl)-8-isopropyl-4,7,10,13-tetraoxo-11-(3-(3-((2,2,4,6,7-pentamethyl-2,3-dihydrobenzofuran-5-yl)sulfonyl)guanidino)propyl)-15,18-dioxa-3,6,9,12-tetraazaicosan-1-oate) LC-MS: MS (ES⁺): RT=1.392 min, m/z=1017.3 [M+H⁺].
Compound 3PEG-RVSF-OH ((2S,5S,8S,11S)-tert-butyl23-amino-2-benzyl-5-(tert-butoxymethyl)-8-isopropyl-4,7,10,13-tetraoxo-11-(3-(3-((2,2,4,6,7-pentamethyl-2,3-dihydrobenzofuran-5-yl)sulfonyl)guanidino)propyl)-15,18,21-trioxa-3,6,9,12-tetraazatricosan-1-oate)¹HNMR (400 MHz, CD₃OD): δ 7.12-7.39 (m, 5H), 4.54-4.62 (m, 2H), 4.44-4.50 (m, 1H), 4.19-4.26 (m, 1H), 4.00-4.14 (m, 2H), 3.51-3.85 (m, 12H), 3.11-3.31 (m, 4H), 2.98-3.09 (m, 4H), 2.47-2.64 (m, 6H), 2.02-2.22 (m, 4H), 1.80-1.95 (m, 1H), 1.68-7.72 (m, 1H), 1.57-1.59 (m, 2H), 1.47 (s, 6H), 1.39 (s, 9H), 1.12-1.21 (m, 9H), 0.96 (dd, 6H, J=6.8, 2.9 Hz). LC-MS: MS (ES⁺): RT=0.914 min, m/z=1061.5 [M+H⁺].
Compound 4PEG-RVSF-OH ((2S,5S,8S,11 S)-tert-butyl26-amino-2-benzyl-5-(tert-butoxymethyl)-8-isopropyl-4,7,10,13-tetraoxo-11-(3-(3-((2,2,4,6,7-pentamethyl-2,3-dihydrobenzofuran-5-yl)sulfonyl)guanidino)propyl)-15,18,21,24-tetraoxa-3,6,9,12-tetraazahexacosan-1-oate)¹HNMR (400 MHz, CD₃OD): δ 7.16-7.34 (m, 5H), 4.05-4.65 (m, 6H), 3.46-3.82 (m, 13H), 3.12-3.32 (m, 6H), 2.98-3.08 (m, 4H), 2.50-2.63 (m, 6H), 2.04-2.18 (m, 4H), 1.79-1.92 (m, 1H), 1.65-1.75 (m, 1H), 1.57-1.59 (m, 2H), 1.45-1.50 (m, 1H), 1.47 (s, 6H), 1.39 (s, 9H), 1.13-1.22 (m, 9H), 0.91-1.03 (m, 6H). LC-MS: MS (ES⁺): RT=0.914 min, m/z=1105.5 [M+H⁺].

### Example 20: Preparation of Amide Compounds from Carboxylic Acid-Containing Target Protein Ligand-Succinates and Amine-Containing Linker-Protein Phosphatase Ligands

Product amide compounds in Table 4 below were prepared from carboxylic acid-containing target protein ligand-succinates and amine-containing linker-protein phosphatase ligands according to the general synthetic procedures below, as described and depicted in the schemes.

### Part I-General Procedure for Amide Coupling and Deprotection

A mixture of carboxylic acid-containing target protein ligand-succinate (˜30 μmol, 1.0 equiv), amine-containing linker-protein phosphatase ligand (˜1.1 equiv), HATU (˜1.2 equiv) and DIEA (˜3.0 equiv) in DMF (0.2 mL) was stirred at 20° C. for 0.5 h. The mixture was purified by prep-HPLC to give the protected amide compound.
A mixture of protected amide compound (˜50 μmol, 1.0 equiv) in H₂O (0.1 mL) and TFA (2 mL) was stirred at 20° C. for 0.5 h. The mixture was concentrated to give a residue. The residue was purified by prep-HPLC to afford the product amide compound as a solid.

### Preparation of Compound I-146

The following scheme depicts the coupling procedure for carboxylic acid-containing target protein ligand-succinate AKTallo-succinate and amine-containing linker-protein phosphatase ligand 0PEG-RVSF-NMe to afford protected amide compound 1, and its subsequent deprotection to afford Compound I-146, according to the procedures described above.

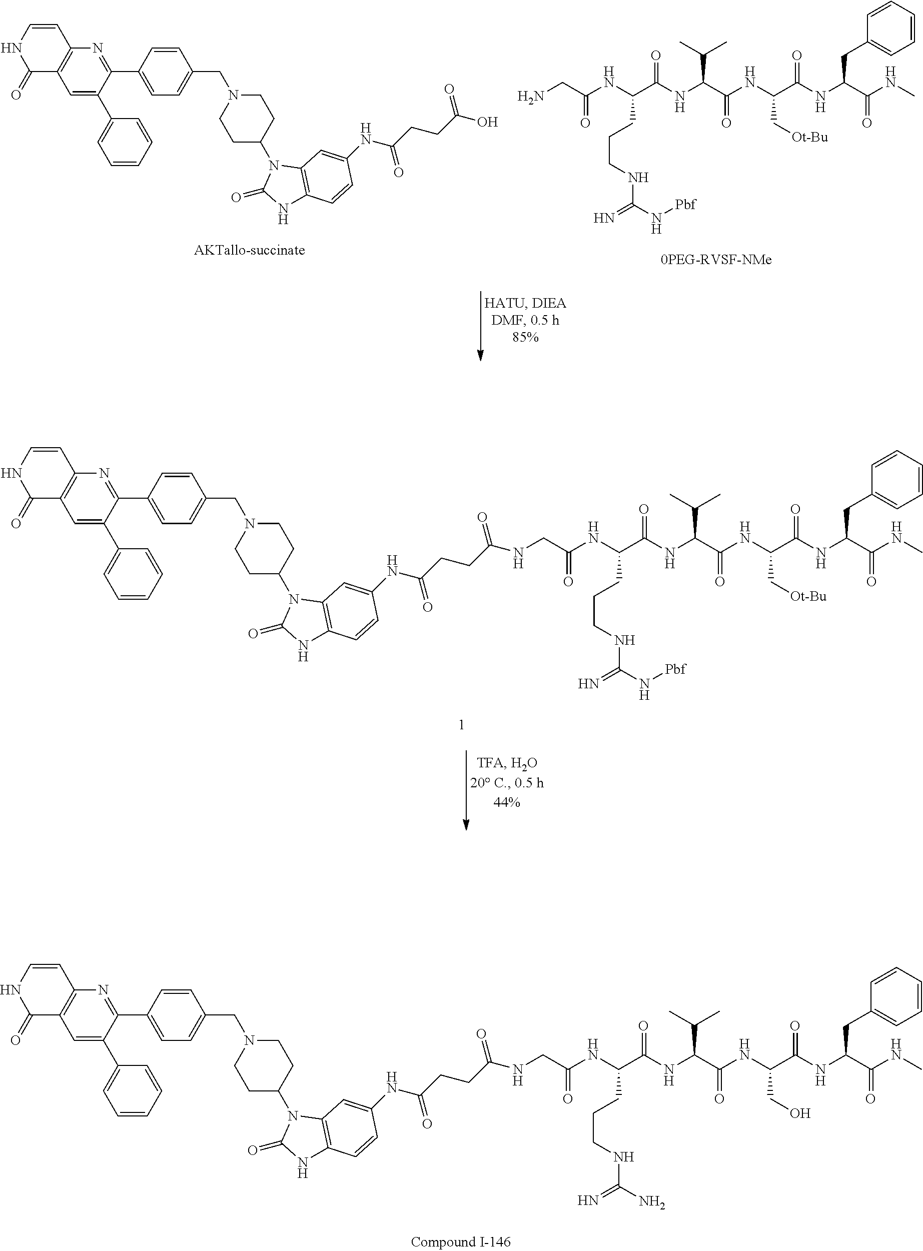

### Physical Characterization Data for Compound 1 and Compound I-146:

Compound 1 (40 mg, 85% yield) was isolated as a yellow solid following purification by prep-HPLC (column: Phenomenex luna C18 150*25 mm*10 μm; mobile phase: [water(0.1% TFA)-ACN]; B %: 30%-60%, 10 min). N-[2-[[(1S)-1-[[(1S)-1-[[(1S)-2-[[(1S)-1-benzyl-2-(methylamino)-2-oxo-ethyl]amino]-1-(tert-butoxymethyl)-2-oxo-ethyl]carbamoyl]-2-methyl-propyl]carbamoyl]-4-[[N-[(2,2,4,6,7-pentamethyl-3H-benzofuran-5-yl)sulfonyl]carbamimidoyl]amino]butyl]amino]-2-oxo-ethyl]-N′-[2-oxo-3-[1-[[4-(5-oxo-3-phenyl-6H-1,6-naphthyridin-2-yl)phenyl]methyl]-4-piperidyl]-1H-benzimidazol-5-yl]butanediamide. LC-MS: MS (ES⁺): RT=0.910 min, m/z=1511.8 [M+H⁺].
Compound I-146 (29 mg, 44% yield, 99.78% purity) was isolated as the HCl salt as a yellow solid following purification by prep-HPLC (column: Phenomenex Synergi C18 150*25*10 μm; mobile phase: [water(0.05% HCl)-ACN]; B %: 17%-37%, 9 min). N-[2-[[(1S)-1-[[(1S)-1-[[(1S)-2-[[(1S)-1-benzyl-2-(methylamino)-2-oxo-ethyl]amino]-1-(hydroxymethyl)-2-oxo-ethyl]carbamoyl]-2-methyl-propyl]carbamoyl]-4-guanidino-butyl]amino]-2-oxo-ethyl]-N′-[2-oxo-3-[1-[[4-(5-oxo-3-phenyl-6H-1,6-naphthyridin-2-yl)phenyl]methyl]-4-piperidyl]-1H-benzimidazol-5-yl]butanediamide. ¹H NMR (400 MHz, CD₃OD): δ 8.97 (s, 1H), 7.75 (d, 1H, J=7.5 Hz), 7.67 (s, 3H), 7.61-7.65 (m, 2H), 7.33-7.39 (m, 3H), 7.27-7.32 (m, 2H), 7.1-7.25 (m, 5H), 7.04-7.09 (m, 1H), 6.99-7.03 (m, 1H), 6.94 (d, 1H, J=7.5 Hz), 4.52 (m, 2H), 4.45 (s, 2H), 4.30-4.40 (m, 2H), 4.06-4.11 (m, 1H), 3.83-3.97 (m, 2H), 3.75-3.81 (m, 1H), 3.68-3.74 (m, 1H), 3.57-3.65 (m, 2H), 3.15 (m, 1H), 3.03 (m, 2H), 2.74-2.97 (m, 5H), 2.58-2.68 (m, 5H), 2.02-2.16 (m, 3H), 1.69-1.88 (m, 2H), 1.52-1.63 (m, 2H), 0.90 (m, 6H); LC-MS: MS (ES⁺): RT=2.265 min, m/z=1202.4 [M+H⁺].
Part II-General Procedure for Preparing RVSF-OH-Containing Product Amide Compounds from Resin-Bound, Amine-Containing (0-2)PEG-RVSF Compounds
Certain RVSF-OH-containing product amide compounds were prepared from resin-bound, amine-containing (0-2)PEG-RVSF compounds (described in Example 17) according to the general synthetic procedures below, as described and depicted in the schemes.
A mixture of compound (0-2)PEG-RVSF-Resin (˜1.6 mmol, 1 equiv), DIEA (˜5 equiv), carboxylic acid-containing target protein ligand-succinate (˜1.1 equiv) and HATU (˜2.5 equiv) in DMF (5 mL) was agitated with N₂ bubbling at 20° C. for 2 h. The solvent was filtered, and the solid was washed with DMF (3×3 mL) and MeOH (3×3 mL). The crude solid in HFIP (10 mL, 20% v\v in DCM) was agitated with bubbling N₂ at 20° C. for 1.5 h. The reaction mixture was filtered and the combined filtrate was concentrated. The resulting residue was purified by prep-HPLC to afford the protected amide compound.
To a solution of the protected amide compound (˜60 μmol) in DCM (300 μL) were added TFA (1.5 mL) and H₂O (300 μL). The mixture was stirred at 20° C. for 6 h and then concentrated. The resulting residue was purified by prep-HPLC to afford the product amide compound (as the HCl salt or free base).

### Preparation of Compound I-179 and Compound I-148

The following scheme depicts the coupling procedure for 1PEG-RVSF-Resin and Chloroalkane-succinate to afford protected amide compound 1, and its subsequent deprotection to afford Compound I-179, according to the procedures described above.

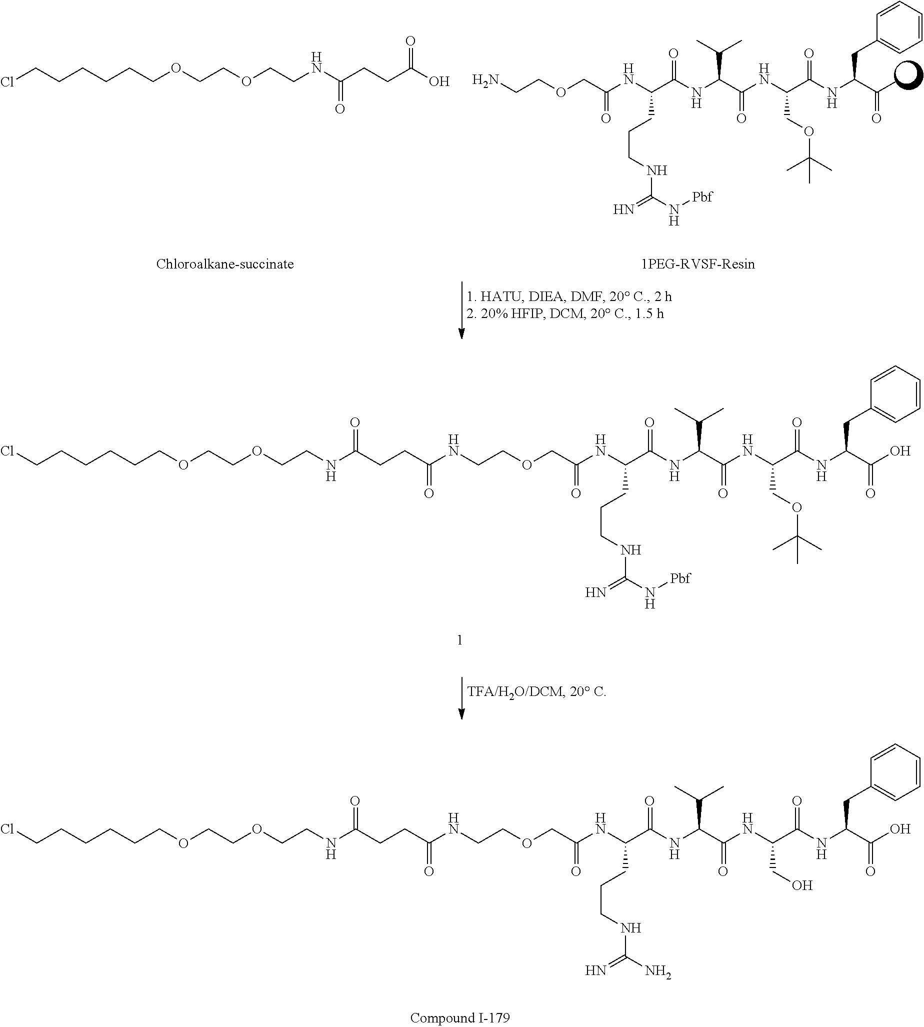

### Physical Characterization Data for Compound I-179:

Compound I-179 ((2S,5S,8S,11S)-2-benzyl-35-chloro-11-(3-guanidinopropyl)-5-(hydroxymethyl)-8-isopropyl-4,7,10,13,19,22-hexaoxo-15,26,29-trioxa-3,6,9,12,18,23-hexaazapentatriacontan-1-oic acid) H NMR (400 MHz, DMSO-d₆) δ 7.06-7.22 (m, 5H), 4.30 (dd, J=8.44, 6.11 Hz, 1H), 4.18 (t, J=5.75 Hz, 1H), 4.01-4.10 (m, 2H), 3.87 (s, 2H), 3.53-3.61 (m, 4H), 3.30-3.50 (m, 10H), 3.12-3.28 (m, 4H), 3.03-3.11 (m, 1H), 2.86-3.02 (m, 3H), 2.25-2.33 (m, 4H), 1.87-1.98 (m, 1H), 1.72-1.85 (m, 1H), 1.60-1.71 (m, 2H), 1.40-1.56 (m, 3H), 1.18-1.39 (m, 6H), 0.78 (dd, J=6.66, 3.73 Hz, 6H). LC-MS: MS (ES⁺): RT=2.15 min, m/z=457.6 [M/2+H⁺], 914.3 [M+H⁺].

The following scheme depicts the coupling procedure for 0PEG-RVSF-Resin and AKTallo-succinate to afford protected amide compound 1, and its subsequent deprotection to afford Compound I-148, according to the procedures described above.

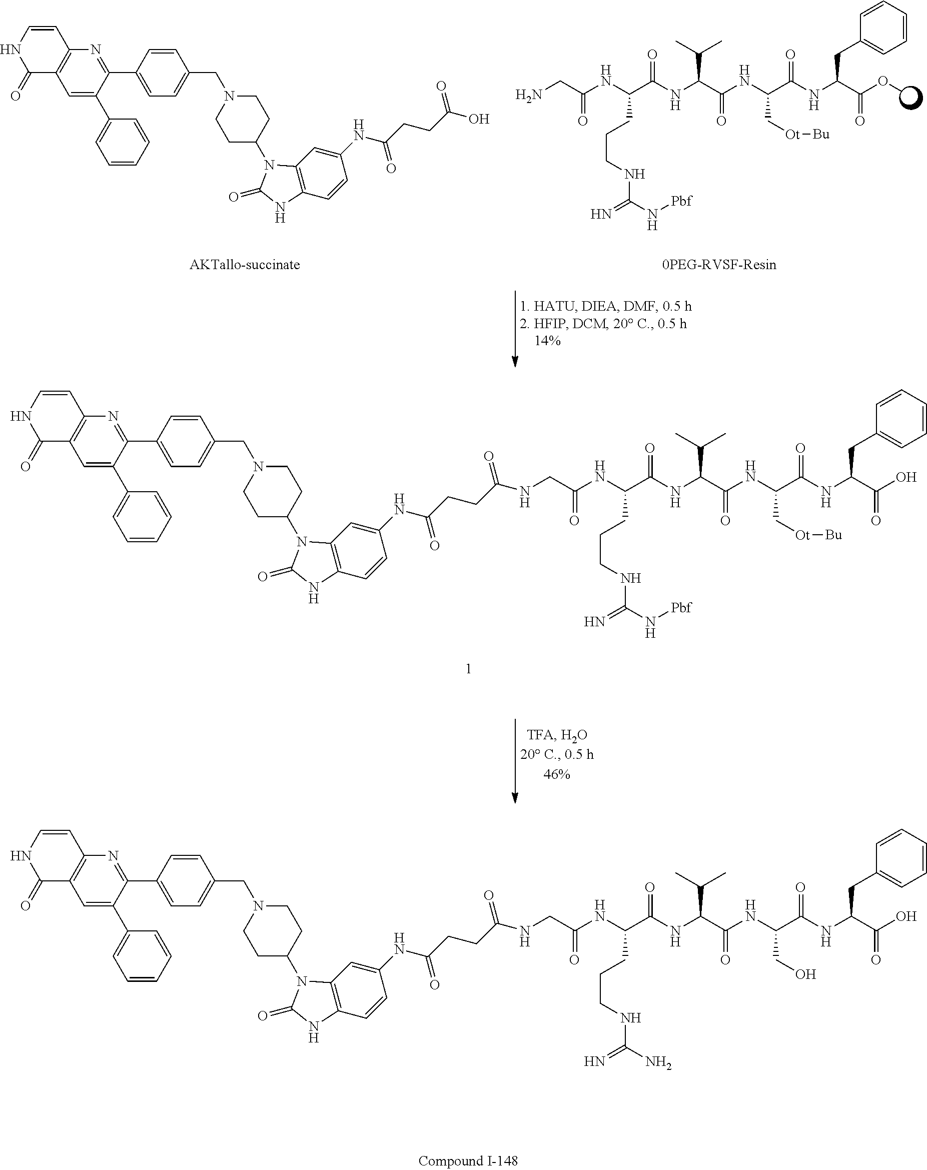

Physical Characterization Data for Compound 1 and Compound I-148:

Compound 1 (32 mg, 14% yield) was isolated as a white solid following purification by prep-HPLC (column: UniSil 3-100 C18 Ultra (150*25 mm*3 um); mobile phase: [water (0.225% FA)-ACN]; B %: 25%-55%, 10 min). (2S)-2-[[(2S)-3-tert-butoxy-2-[[(2S)-3-methyl-2-[[(2S)-2-[[2-[[4-oxo-4-[[2-oxo-3-[1-[[4-(5-oxo-3-phenyl-6H-1,6-naphthyridin-2-yl)phenyl]methyl]-4-piperidyl]-1H-benzimidazol-5-yl]amino]butanoyl]amino]acetyl]amino]-5-[[N-[(2,2,4,6,7-pentamethyl-3H-benzofuran-5-yl)sulfonyl]carbamimidoyl]amino]pentanoyl]amino]butanoyl]amino]propanoyl]amino]-3-phenyl-propanoic acid. LC-MS: MS (ES⁺): RT=0.807 min, m/z=1498.8 [M+H⁺].
Compound I-148 (12 mg, 46% yield, 98.34% purity, HCl salt) was isolated as a yellow solid following purification by prep-HPLC (column: Phenomenex Synergi C18 15025103μm; mobile phase: [water(0.05% HCl)-ACN]; B %: 18%-38%, 9 min). (2S)-2-[[(2S)-2-[[(2S)-2-[[(2S)-5-guanidino-2-[[2-[[4-oxo-4-[[2-oxo-3-[1-[[4-(5-oxo-3-phenyl-6H-1,6-naphthyridin-2-yl)phenyl]methyl]-4-piperidyl]-1H-benzimidazol-5-yl]amino]butanoyl]amino]acetyl]amino]pentanoyl]amino]-3-methyl-butanoyl]amino]-3-hydroxy-propanoyl]amino]-3-phenyl-propanoic acid. ¹H NMR (400 MHz, CD₃OD): δ 8.89 (s, 1H), 7.56-7.75 (m, 6H), 7.32-7.39 (m, 3H), 7.15-7.30 (m, 7H), 6.97-7.09 (m, 2H), 6.93 (d, 1H, J=7.46 Hz), 4.60-4.70 (m, 1H), 4.50-4.67 (m, 1H), 4.31-4.48 (m, 4H), 4.18 (d, 1H, J=6.5 Hz), 3.82-3.96 (m, 2H), 3.76 (d, 2H, J=5.4 Hz), 3.62 (m, 2H), 3.35 (m, 1H), 3.11-3.21 (m, 1H), 2.94-3.08 (m, 3H), 2.72-2.91 (m, 4H), 2.55-2.69 (m, 2H), 1.99-2.19 (m, 3H), 1.66-1.87 (m, 2H), 1.47-1.63 (m, 2H), 0.90 (d, 6H, J=6.6 Hz); LC-MS: MS (ES⁺): RT=1.945 min, m/z=1189.4 [M+H⁺].

Part III-General Procedure for Preparing RVSF-NMe- and RVSF-NOct-Containing Product Amide Compounds from Resin-Bound, Amine-Containing (0-2)PEG-RVSF Compounds

Certain RVSF-NMe- and RVSF-NOct-containing product amide compounds were prepared from resin-bound, amine-containing (0-2)PEG-RVSF compounds via the protected amide compound (described in Part II, above) according to the general synthetic procedures below, as described and depicted in the schemes.
To a solution of the protected amide compound (˜60 μmol, 1.0 equiv), methylamine or octylamine (5.0 equiv) in DMF (3 mL) were added HATU (1.0 equiv) and DIEA (9.0 equiv). The mixture was stirred at 20° C. for 12 h. The organic solvent was concentrated to give a residue.
The residue was purified by prep-HPLC (a 5 micron Waters Xbridge 150×25 mm column with NH₄HCO₃ modified water/acetonitrile mobile phase).
To a solution of the purified intermediate (˜60 μmol) in DCM (300 μL) were added TFA (1.5 mL) and H₂O (300 μL). The mixture was stirred at 20° C. for 6 h and then concentrated. The resulting residue was purified by prep-HPLC to afford the product amide compound (HCl salt or free base).

### Preparation of Compound I-177

The following scheme depicts the procedure for converting chloroalkane compound 1 (from Part II, above) to Compound I-177, according to the procedures described above.

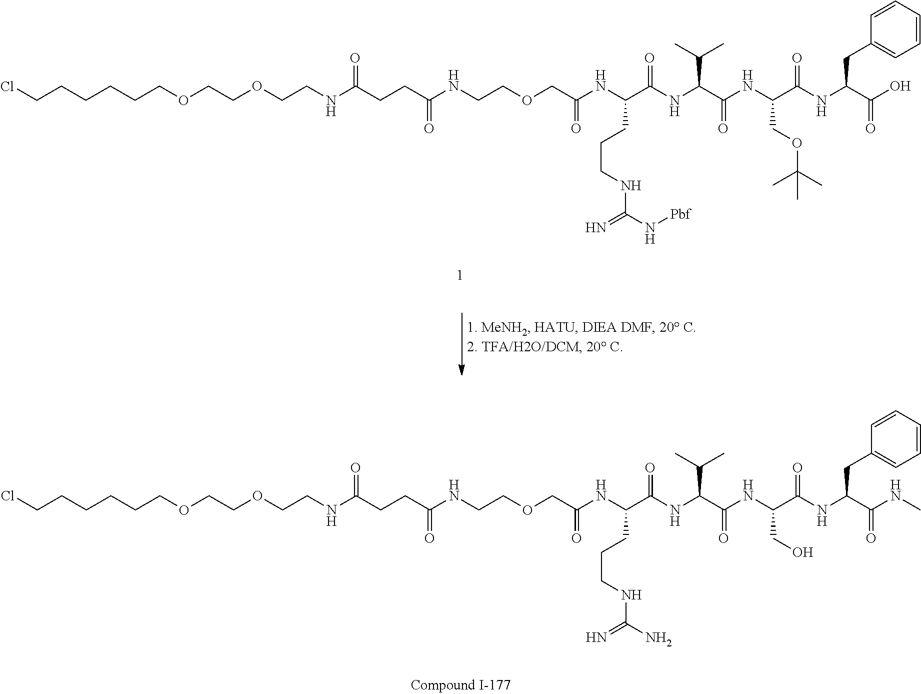

### Physical Characterization Data for Compound I-177:

Compound I-177 HN NH₂ Compound I-177 (N1-((4S,7S,10 OS, 13 S)-4-benzyl-13-(3-guanidinopropyl)-7-(hydroxymethyl)-10-isopropyl-3,6,9,12,15-pentaoxo-17-oxa-2,5,8,11,14-pentaazanonadecan-19-yl)-N4-(2-(2-((6-chlorohexyl)oxy)ethoxy)ethyl)succinamide)¹H NMR (400 MHz, DMSO-d₆) δ 7.18-7.24 (m, 2H), 7.12-7.17 (m, 3H), 4.35 (dd, J=9.05, 5.01 Hz, 2H), 4.23 (t, J=6.11 Hz, 1H), 4.07 (d, J=6.72 Hz, 1H), 3.88 (s, 2H), 3.52-3.59 (m, 3H), 3.41-3.52 (m, 8H), 3.30-3.40 (m, 5H), 3.19-3.25 (m, 2H), 3.15 (t, J=5.69 Hz, 2H), 2.94-3.05 (m, 3H), 2.78 (dd, J=13.82, 9.17 Hz, 1H), 2.30 (s, 3H), 1.92 (dq, J=13.65, 6.94 Hz, 1H), 1.12-1.76 (m, 14H), 0.75 (d, J=6.72 Hz, 6H). LC-MS: MS (ES⁺): RT=2.46 min, m/z=927.3, 929.3 [M+H⁺].

### Part IV-Exemplary Amide Compounds Prepared According to the General Procedures

The product amide compounds described in Table 4 below were prepared from carboxylic acid-containing target protein ligand-succinates and amine-containing linker-protein phosphatase ligands according to the procedures described in Parts I-III. Chemical structures for abbreviations used in the description of product amide compounds in Table 4 are provided in Table 5 below.

TABLE 4
Exemplary Product Amide Compounds

| | | | Linker-Protein | | | | |
|---|---|---|---|---|---|---|---|
| | | Target Protein | Phosphatase | | | | |
| | | Ligand-Succinate | Ligand Used to | | | | |
| Com- | | Used to Prepare | Prepare Product | | LC/MS | | |
| pound | Product Amide | Product Amide | Amide | | MH + | | |
| No. | Compound | Compound | Compound | MW | (obs) | RT | Method |
| I-146 | (AKTallo-succinate)- | AKTallo-succinate | 0PEG-RVSF-NMe | 1202.4 | 1202 | 2.27 | 01 |
| | (0PEG-RVSF-NMe) | | | | | | |
| I-147 | (AKTallo-succinate)- | AKTallo-succinate | 0PEG-RVSF-NOct | 1300.6 | 1300 | 2.33 | 10 |
| | (0PEG-RVSF-NOct) | | | | | | |
| I-148 | (AKTallo-succinate)- | AKTallo-succinate | 0PEG-RVSF-OH | 1189.3 | 1189 | 1.95 | 10 |
| | (0PEG-RVSF-OH) | | | | | | |
| I-149 | (AKTallo-succinate)- | AKTallo-succinate | 1PEG-RVSF-NMe | 1246.4 | 1248 | 1.95 | 10 |
| | (1PEG-RVSF-NMe) | | | | | | |
| I-150 | (AKTallo-succinate)- | AKTallo-succinate | 1PEG-RVSF-NOct | 1344.6 | 1346 | 2.32 | 10 |
| | (1PEG-RVSF-NOct) | | | | | | |
| I-151 | (AKTallo-succinate)- | AKTallo-succinate | 1PEG-RVSF-OH | 1233.4 | 1234 | 2.29 | 01 |
| | (1PEG-RVSF-OH) | | | | | | |
| I-152 | (AKTallo-succinate)- | AKTallo-succinate | 2PEG-RVSF-NMe | 1290.5 | 1291 | 1.97 | 10 |
| | (2PEG-RVSF-NMe) | | | | | | |
| I-153 | (AKTallo-succinate)- | AKTallo-succinate | 2PEG-RVSF-NOct | 1388.7 | 1389 | 2.32 | 10 |
| | (2PEG-RVSF-NOct) | | | | | | |
| I-154 | (AKTallo-succinate)- | AKTallo-succinate | 2PEG-RVSF-OH | 1277.4 | 1278 | 1.97 | 10 |
| | (2PEG-RVSF-OH) | | | | | | |
| I-155 | (AKTallo-succinate)- | AKTallo-succinate | 3PEG-RVSF-NMe | 1334.5 | 1335 | 2.31 | 01 |
| | (3PEG-RVSF-NMe) | | | | | | |
| I-156 | (AKTallo-succinate)- | AKTallo-succinate | 3PEG-RVSF-NOct | 1432.7 | 1432 | 2.34 | 10 |
| | (3PEG-RVSF-NOct) | | | | | | |
| I-157 | (AKTallo-succinate)- | AKTallo-succinate | 4PEG-RVSF-NMe | 1378.6 | 1378 | 2.00 | 10 |
| | (4PEG-RVSF-NMe) | | | | | | |
| I-158 | (AKTallo-succinate)- | AKTallo-succinate | 4PEG-RVSF-NOct | 1476.8 | 739 | 2.35 | 10 |
| | (4PEG-RVSF-NOct) | | | | [M/2 + H] | | |
| I-159 | (AKTcomp-succinate)- | AKTcomp- | 0PEG-RVSF-NMe | 1174.8 | 1174 | 2.12 | 01 |
| | (0PEG-RVSF-NMe) | succinate | | | | | |
| I-160 | (AKTcomp-succinate)- | AKTcomp- | 0PEG-RVSF-NOct | 1273.0 | 1275 | 2.20 | 10 |
| | (0PEG-RVSF-NOct) | succinate | | | | | |
| I-161 | (AKTcomp-succinate)- | AKTcomp- | 0PEG-RVSF-OH | 1161.8 | 1163 | 1.82 | 10 |
| | (0PEG-RVSF-OH) | succinate | | | | | |
| I-162 | (AKTcomp-succinate)- | AKTcomp- | 1PEG-RVSF-NMe | 1218.9 | 1221 | 2.15 | 01 |
| | (1PEG-RVSF-NMe) | succinate | | | | | |
| I-163 | (AKTcomp-succinate)- | AKTcomp- | 1PEG-RVSF-NOct | 1317.1 | 1317 | 2.21 | 10 |
| | (1PEG-RVSF-NOct) | succinate | | | | | |
| I-164 | (AKTcomp-succinate)- | AKTcomp- | 1PEG-RVSF-OH | 1205.8 | 1207 | 1.83 | 10 |
| | (1PEG-RVSF-OH) | succinate | | | | | |
| I-165 | (AKTcomp-succinate)- | AKTcomp- | 2PEG-RVSF-NMe | 1262.9 | 1266 | 1.81 | 10 |
| | (2PEG-RVSF-NMe) | succinate | | | | | |
| I-166 | (AKTcomp-succinate)- | AKTcomp- | 2PEG-RVSF-NOct | 1361.1 | 1361 | 2.22 | 10 |
| | (2PEG-RVSF-NOct) | succinate | | | | | |
| I-167 | (AKTcomp-succinate)- | AKTcomp- | 2PEG-RVSF-OH | 1249.9 | 1252 | 1.84 | 10 |
| | (2PEG-RVSF-OH) | succinate | | | | | |
| I-168 | (AKTcomp-succinate)- | AKTcomp- | 3PEG-RVSF-NMe | 1307.0 | 1307 | 1.85 | 01 |
| | (3PEG-RVSF-NMe) | succinate | | | | | |
| I-169 | (AKTcomp-succinate)- | AKTcomp- | 3PEG-RVSF-NOct | 1405.2 | 1405 | 2.24 | 10 |
| | (3PEG-RVSF-NOct) | succinate | | | | | |
| I-170 | (AKTcomp-succinate)- | AKTcomp- | 3PEG-RVSF-OH | 1293.9 | 1297 | 1.85 | 10 |
| | (3PEG-RVSF-OH) | succinate | | | | | |
| I-171 | (AKTcomp-succinate)- | AKTcomp- | 4PEG-RVSF-NMe | 1351.0 | 1351 | 1.86 | 10 |
| | (4PEG-RVSF-NMe) | succinate | | | | | |
| I-172 | (AKTcomp-succinate)- | AKTcomp- | 4PEG-RVSF-NOct | 1449.2 | 1450 | 2.20 | 10 |
| | (4PEG-RVSF-NOct) | succinate | | | | | |
| I-173 | (AKTcomp-succinate)- | AKTcomp- | 4PEG-RVSF-OH | 1338.0 | 669 | 1.88 | 10 |
| | (4PEG-RVSF-OH) | succinate | | | [M/2 + H] | | |
| I-174 | (Chloroalkane-succinate)- | Chloroalkane- | 0PEG-RVSF-NMe | 883.5 | 883 | 2.11 | 10 |
| | (0PEG-RVSF-NMe) | succinate | | | | | |
| I-175 | (Chloroalkane-succinate)- | Chloroalkane- | 0PEG-RVSF-NOct | 981.7 | 981 | 2.89 | 01 |
| | (0PEG-RVSF-NOct) | succinate | | | | | |
| I-176 | (Chloroalkane-succinate)- | Chloroalkane- | 0PEG-RVSF-OH | 870.4 | 870 | 2.13 | 10 |
| | (0PEG-RVSF-OH) | succinate | | | | | |
| I-177 | (Chloroalkane-succinate)- | Chloroalkane- | 1PEG-RVSF-NMe | 927.5 | 927 | 2.46 | 01 |
| | (1PEG-RVSF-NMe) | succinate | | | | | |
| I-178 | (Chloroalkane-succinate)- | Chloroalkane- | 1PEG-RVSF-NOct | 1025.7 | 1026 | 2.89 | 01 |
| | (1PEG-RVSF-NOct) | succinate | | | | | |
| I-179 | (Chloroalkane-succinate)- | Chloroalkane- | 1PEG-RVSF-OH | 914.5 | 914 | 2.15 | 10 |
| | (1PEG-RVSF-OH) | succinate | | | | | |
| I-180 | (Chloroalkane-succinate)- | Chloroalkane- | 2PEG-RVSF-NMe | 971.6 | 972 | 2.13 | 10 |
| | (2PEG-RVSF-NMe) | succinate | | | | | |
| I-181 | (Chloroalkane-succinate)- | Chloroalkane- | 2PEG-RVSF-NOct | 1069.8 | 1070 | 2.89 | 01 |
| | (2PEG-RVSF-NOct) | succinate | | | | | |
| I-182 | (Chloroalkane-succinate)- | Chloroalkane- | 2PEG-RVSF-OH | 958.5 | 958 | 2.94 | 00 |
| | (2PEG-RVSF-OH) | succinate | | | | | |
| I-183 | (Chloroalkane-succinate)- | Chloroalkane- | 3PEG-RVSF-NMe | 1015.6 | 1019 | 2.15 | 10 |
| | (3PEG-RVSF-NMe) | succinate | | | | | |
| I-184 | (Chloroalkane-succinate)- | Chloroalkane- | 3PEG-RVSF-NOct | 1113.8 | 1114 | 2.90 | 01 |
| | (3PEG-RVSF-NOct) | succinate | | | | | |
| I-185 | (Chloroalkane-succinate)- | Chloroalkane- | 3PEG-RVSF-OH | 1002.6 | 1002 | 2.49 | 01 |
| | (3PEG-RVSF-OH) | succinate | | | | | |
| I-186 | (Chloroalkane-succinate)- | Chloroalkane- | 4PEG-RVSF-NMe | 1059.7 | 1063 | 2.15 | 10 |
| | (4PEG-RVSF-NMe) | succinate | | | | | |
| I-187 | (Chloroalkane-succinate)- | Chloroalkane- | 4PEG-RVSF-NOct | 1157.9 | 1161 | 2.88 | 01 |
| | (4PEG-RVSF-NOct) | succinate | | | | | |
| I-188 | (Chloroalkane-succinate)- | Chloroalkane- | 4PEG-RVSF-OH | 1046.6 | 1047 | 2.95 | 3 |
| | (4PEG-RVSF-OH) | succinate | | | | | |
| I-189 | (TBK1-succinate)-(0PEG- | TBK1-succinate | 0PEG-RVSF-NMe | 1137.1 | 1137 | 2.27 | 01 |
| | RVSF-NMe) | | | | | | |
| I-190 | (TBK1-succinate)-(0PEG- | TBK1-succinate | 0PEG-RVSF-NOct | 1235.3 | 1236 | 1.93 | 25 |
| | RVSF-NOct) | | | | | | |
| I-191 | (TBK1-succinate)-(0PEG- | TBK1-succinate | 0PEG-RVSF-OH | 1124.1 | 563 | 2.29 | 01 |
| | RVSF-OH) | | | | [M/2 + H] | | |
| I-192 | (TBK1-succinate)-(1PEG- | TBK1-succinate | 1PEG-RVSF-NMe | 1181.2 | 1180 | 2.29 | 01 |
| | RVSF-NMe) | | | | | | |
| I-193 | (TBK1-succinate)-(1PEG- | TBK1-succinate | 1PEG-RVSF- | 1279.4 | 641 | 1.92 | 25 |
| | RVSF-NOct) | | NOct | | [M/2 + H] | | |
| I-194 | (TBK1-succinate)-(1PEG- | TBK1-succinate | 1PEG-RVSF-OH | 1168.1 | 1167 | 2.30 | 01 |
| | RVSF-OH) | | | | | | |
| I-195 | (TBK1-succinate)-(2PEG- | TBK1-succinate | 2PEG-RVSF-NMe | 1225.2 | 1224 | 2.30 | 01 |
| | RVSF-NMe) | | | | | | |
| I-196 | (TBK1-succinate)-(2PEG- | TBK1-succinate | 2PEG-RVSF- | 1323.4 | 663 | 2.38 | 10 |
| | RVSF-NOct) | | NOct | | [M/2 + H] | | |
| I-197 | (TBK1-succinate)-(2PEG- | TBK1-succinate | 2PEG-RVSF-OH | 1212.2 | 1213 | 2.32 | 01 |
| | RVSF-OH) | | | | | | |
| I-198 | (TBK1-succinate)-(3PEG- | TBK1-succinate | 3PEG-RVSF-NMe | 1269.3 | 1269 | 2.30 | 01 |
| | RVSF-NMe) | | | | | | |
| I-199 | (TBK1-succinate)-(3PEG- | TBK1-succinate | 3PEG-RVSF- | 1367.5 | 685 | 2.35 | 10 |
| | RVSF-NOct) | | NOct | | [M/2 + H] | | |
| I-200 | (TBK1-succinate)-(3PEG- | TBK1-succinate | 3PEG-RVSF-OH | 1256.3 | 1256 | 1.98 | 10 |
| | RVSF-OH) | | | | | | |
| I-201 | (TBK1-succinate)-(4PEG- | TBK1-succinate | 4PEG-RVSF-NMe | 1313.3 | 1313 | 2.31 | 01 |
| | RVSF-NMe) | | | | | | |
| I-202 | (TBK1-succinate)-(4PEG- | TBK1-succinate | 4PEG-RVSF-NOct | 1411.5 | 1414 | 2.38 | 10 |
| | RVSF-NOct) | | | | | | |
| I-203 | (TBK1-succinate)-(4PEG- | TBK1-succinate | 4PEG-RVSF-OH | 1300.3 | 1299 | 1.99 | 10 |
| | RVSF-OH) | | | | | | |

TABLE 5
Chemical Structures Corresponding to Abbreviations Used in the Description of the Product Amide Compounds

| Abbreviation | Chemical Structure |
|---|---|
| (AKTallo-succinate)- | 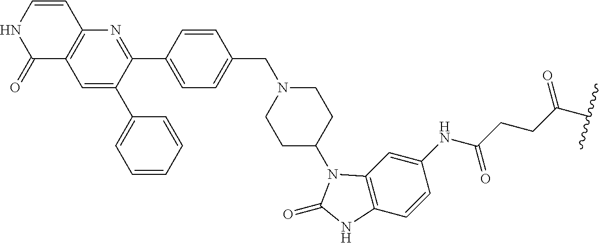 [SMILES: *C(=O)CCC(=O)Nc1ccc2[nH]c(=O)n(C3CCN(Cc4ccc(-c5nc6cc[nH]c(=O)c6cc5-c5ccccc5)cc4)CC3)c2c1] |
| (AKTcomp-succinate)- | 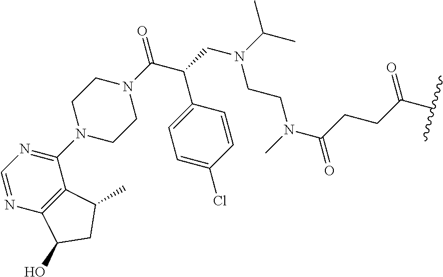 [SMILES: *C(=O)CCC(=O)N(C)CCN(C[C@@H](C(=O)N1CCN(c2ncnc3c2[C@H](C)C[C@H]3O)CC1)c1ccc(Cl)cc1)C(C)C] |
| (Chloroalkane-succinate)- | 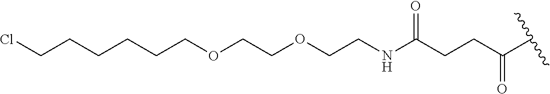 [SMILES: *C(=O)CCC(=O)NCCOCCOCCCCCCCl] |
| (TBK1-succinate)- | 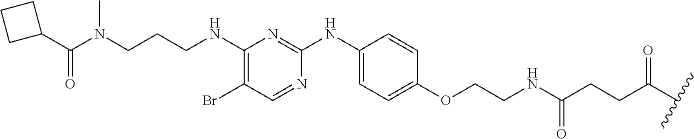 [SMILES: *C(=O)CCC(=O)NCCOc1ccc(Nc2ncc(Br)c(NCCCN(C)C(=O)C3CCC3)n2)cc1] |
| -(0PEG-RVSF-NMe) | 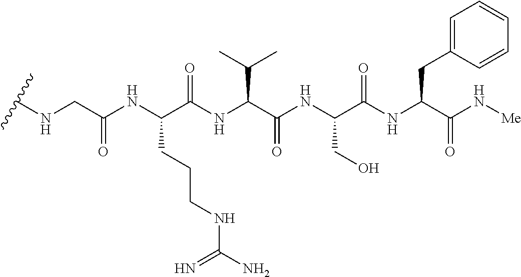 [SMILES: *NCC(=O)N[C@@H](CCCNC(=N)N)C(=O)N[C@H](C(=O)N[C@@H](CO)C(=O)N[C@@H](Cc1ccccc1)C(=O)NC)C(C)C] |
| -(1PEG-RVSF-NMe) | 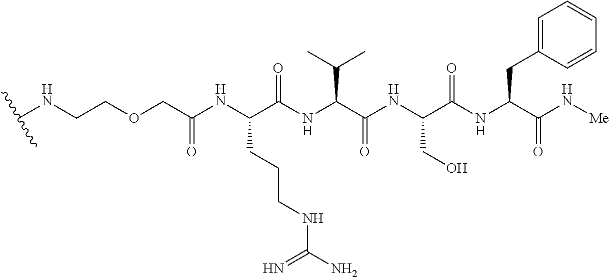 [SMILES: *NCCOCC(=O)N[C@@H](CCCNC(=N)N)C(=O)N[C@H](C(=O)N[C@@H](CO)C(=O)N[C@@H](Cc1ccccc1)C(=O)NC)C(C)C] |
| -(2PEG-RVSF-NMe) | 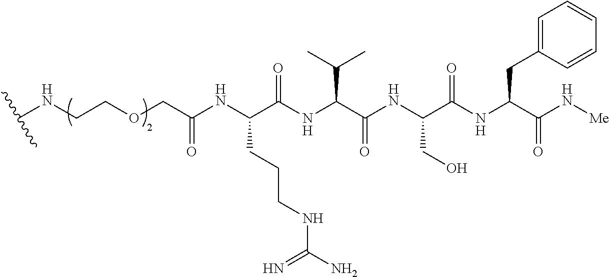 |
| -(3PEG-RVSF-NMe) | 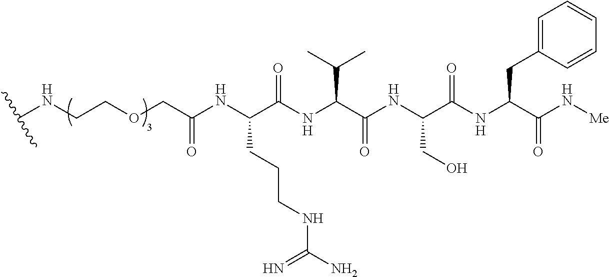 |
| -(4PEG-RVSF-NMe) | 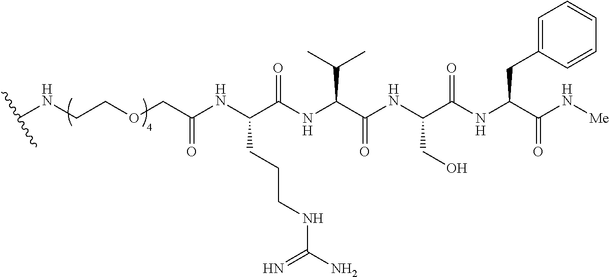 |
| -(0PEG-RVSF-NOct) | 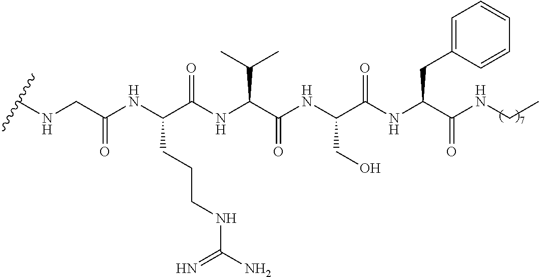 |
| -(1PEG-RVSF-NOct) | 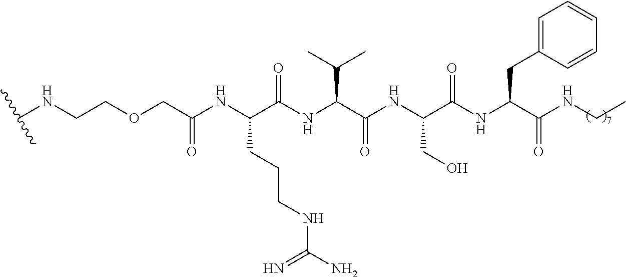 |
| -(2PEG-RVSF-NOct) | 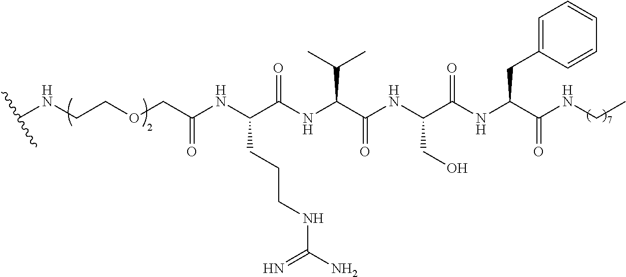 |
| -(3PEG-RVSF-NOct) | 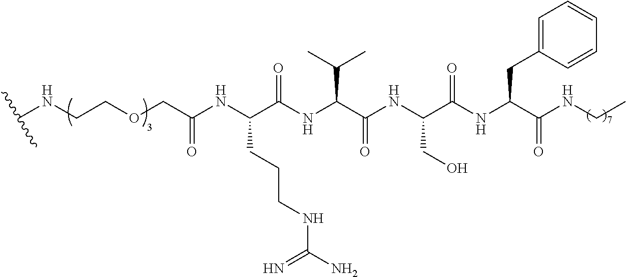 |
| -(4PEG-RVSF-NOct) | 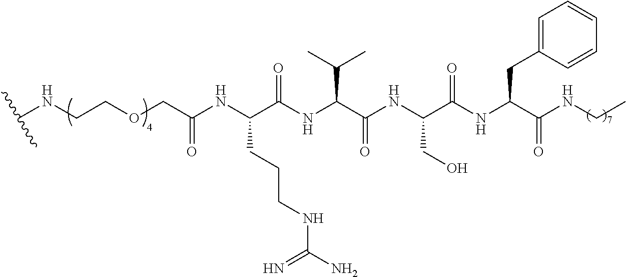 |
| -(0PEG-RVSF-OH) | 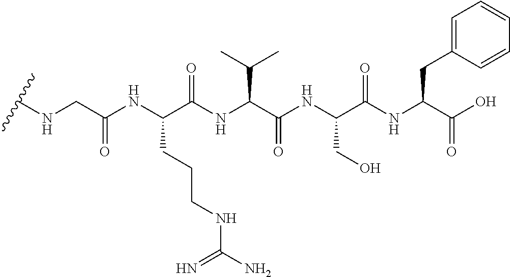 [SMILES: *NCC(=O)N[C@@H](CCCNC(=N)N)C(=O)N[C@H](C(=O)N[C@@H](CO)C(=O)N[C@@H](Cc1ccccc1)C(=O)O)C(C)C] |
| -(1PEG-RVSF-OH) | 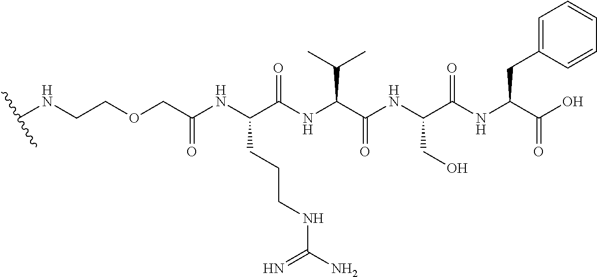 [SMILES: *NCCOCC(=O)N[C@@H](CCCNC(=N)N)C(=O)N[C@H](C(=O)N[C@@H](CO)C(=O)N[C@@H](Cc1ccccc1)C(=O)O)C(C)C] |
| -(2PEG-RVSF-OH) | 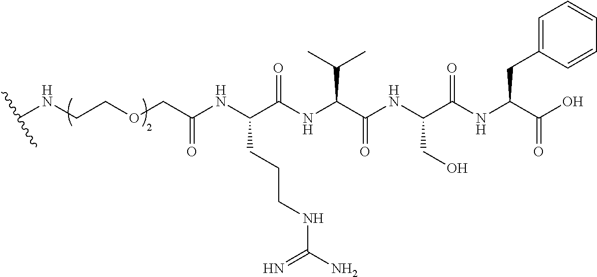 |
| -(3PEG-RVSF-OH) | 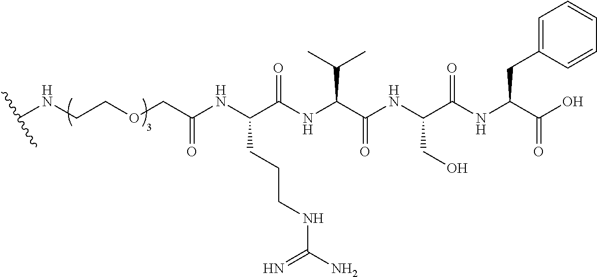 |
| -(4PEG-RVSF-OH) | 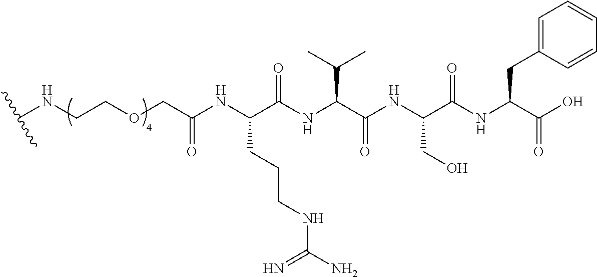 |

### Example 21: Preparation of Product Amide Compounds from Carboxylic Acid-Containing Target Protein Ligands and Amine-Containing Linker-Protein Phosphatase Ligands

The product amide compounds in Table 6 below were prepared from carboxylic acid-containing target protein ligands and amine-containing linker-protein phosphatase ligands according to the general synthetic procedures described in Example 20. Target protein ligand BRD4 is commercially available (CAS RN 202592-23-2) and has the chemical structure

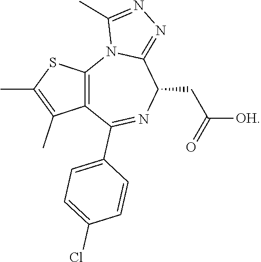 [SMILES: Cc1sc2c(c1C)C(c1ccc(Cl)cc1)=N[C@@H](CC(=O)O)c1nnc(C)n1-2]

Chemical structures for abbreviations used in the description of product amide compounds in Table 6 are depicted in Table 5 above. The chemical structure for abbreviation “(BRD4)-” used in the description of the product amide compounds in Table 6 is

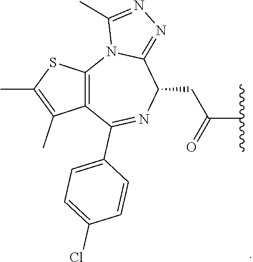 [SMILES: *C(=O)C[C@@H]1N=C(c2ccc(Cl)cc2)c2c(sc(C)c2C)-n2c(C)nnc21]

TABLE 6
Exemplary Product Amide Compounds

| | | | Linker-Protein | | | | |
|---|---|---|---|---|---|---|---|
| | | Target Protein | Phosphatase | | | | |
| | | Ligand | Ligand Used to | | | | |
| Com- | | Used to Prepare | Prepare Product | | LC/MS | | |
| pound | Product Amide | Product Amide | Amide | | MH + | | |
| No. | Compound | Compound | Compound | MW | (obs) | RT | Method |
| I-204 | (BRD4)-(0PEG-RVSF-NMe) | BRD4 | 0PEG-RVSF-NMe | 961 | 960 | 2.25 | 10 |
| I-205 | (BRD4)-(1PEG-RVSF-NMe) | BRD4 | 1PEG-RVSF-NMe | 1005 | 1004 | 2.25 | 10 |
| I-206 | (BRD4)-(2PEG-RVSF-NMe) | BRD4 | 2PEG-RVSF-NMe | 1049 | 1051 | 2.27 | 10 |
| I-207 | (BRD4)-(3PEG-RVSF-NMe) | BRD4 | 3PEG-RVSF-NMe | 1093 | 1095 | 2.28 | 10 |
| I-208 | (BRD4)-(4PEG-RVSF-NMe) | BRD4 | 4PEG-RVSF-NMe | 1137 | 1137 | 2.29 | 10 |
| I-209 | (BRD4)-(0PEG-RVSF-NOct) | BRD4 | 0PEG-RVSF-NOct | 1059 | 1059 | 2.68 | 10 |
| I-210 | (BRD4)-(1PEG-RVSF-NOct) | BRD4 | 1PEG-RVSF-NOct | 1103 | 1104 | 2.67 | 10 |
| I-211 | (BRD4)-(2PEG-RVSF-NOct) | BRD4 | 2PEG-RVSF-NOct | 1147 | 1148 | 2.68 | 10 |
| I-212 | (BRD4)-(3PEG-RVSF-NOct) | BRD4 | 3PEG-RVSF-NOct | 1191 | 1194 | 2.68 | 10 |
| I-213 | (BRD4)-(4PEG-RVSF-NOct) | BRD4 | 4PEG-RVSF-NOct | 1235 | 1234 | 2.69 | 10 |
| I-214 | (BRD4)-(0PEG-RVSF-OH) | BRD4 | 0PEG-RVSF-OH | 948 | 947 | 2.25 | 10 |
| I-215 | (BRD4)-(1PEG-RVSF-OH) | BRD4 | 1PEG-RVSF-OH | 992 | 992 | 2.26 | 10 |
| I-216 | (BRD4)-(2PEG-RVSF-OH) | BRD4 | 2PEG-RVSF-OH | 1036 | 1039 | 2.27 | 10 |
| I-217 | (BRD4)-(3PEG-RVSF-OH) | BRD4 | 3PEG-RVSF-OH | 1080 | 1082 | 2.28 | 10 |
| I-218 | (BRD4)-(4PEG-RVSF-OH) | BRD4 | 4PEG-RVSF-OH | 1124 | 1125 | 2.29 | 10 |

### Example 22: Preparation of Compounds (0-4)PEG-BRD4 (Linker-Target Protein Ligand)

The title compounds were prepared according to the general synthetic procedures below, as described and depicted in the scheme. Target protein ligand BRD4 is commercially available (CAS RN 202592-23-2).

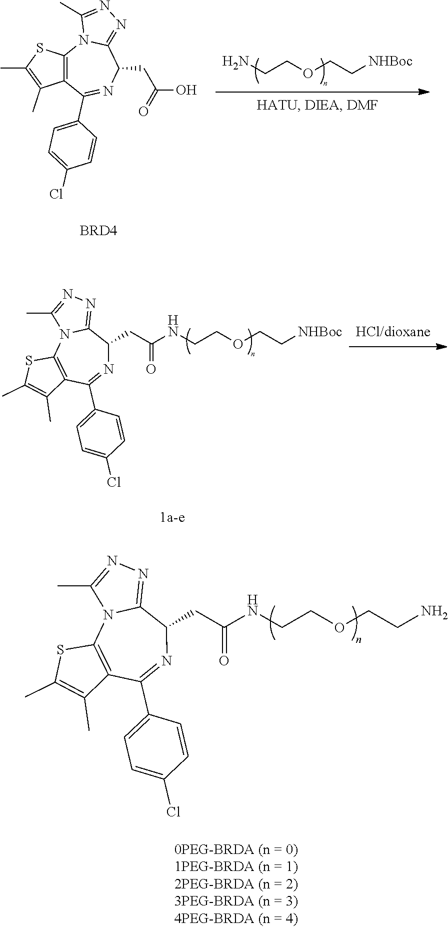

### Part I-General Procedure for the Preparation of Compounds 1a-e

To a solution of compound BRD4 (0.50 g, 1.3 mmol, 1.0 equiv) and Boc-PEG-NH₂ (1.3 mmol, 1.0 equiv) in DMF (3 mL) was added DIEA (322 mg, 2.49 mmol, 435 μL, 2.0 equiv) and HATU (570 mg, 1.50 mmol, 1.2 equiv). The mixture was stirred at 25° C. for 1 h. The reaction mixture was purified by prep-HPLC on a Waters Xbridge C18 150*50 mm*10 μm column with acetonitrile and NH₄HCO₃-modified water as mobile phase to afford compounds 1a-e as white solids.
Physical characterization data for compounds 1a-e:
Compound 1a ((S)-tert-butyl (2-(2-(4-(4-chlorophenyl)-2,3,9-trimethyl-6H-thieno[3,2-f][1,2,4]triazolo[4,3-a][1,4]diazepin-6-yl)acetamido)ethyl)carbamate) LC-MS: MS (ES⁺): RT=0.955 min, m/z=543.4 [M+H⁺].
Compound 1b ((S)-tert-butyl (2-(2-(2-(4-(4-chlorophenyl)-2,3,9-trimethyl-6H-thieno[3,2-f][1,2,4]triazolo[4,3-a][1,4]diazepin-6-yl)acetamido)ethoxy)ethyl)carbamate) LC-MS: MS (ES⁺): RT=0.961 min, m/z=587.4 [M+H⁺].
Compound 1c ((S)-tert-butyl (2-(2-(2-(2-(4-(4-chlorophenyl)-2,3,9-trimethyl-6H-thieno[3,2-f][1,2,4]triazolo[4,3-a][1,4]diazepin-6-yl)acetamido)ethoxy)ethoxy)ethyl)carbamate) LC-MS: MS (ES⁺): RT=0.957 min, m/z=631.4 [M+H⁺].
Compound 1d ((S)-tert-butyl (1-(4-(4-chlorophenyl)-2,3,9-trimethyl-6H-thieno[3,2-f][1,2,4]triazolo[4,3-a][1,4]diazepin-6-yl)-2-oxo-6,9,12-trioxa-3-azatetradecan-14-yl)carbamate) LC-MS: MS (ES⁺): RT=0.957 min, m/z=675.4 [M+H⁺].
Compound 1e ((S)-tert-butyl (1-(4-(4-chlorophenyl)-2,3,9-trimethyl-6H-thieno[3,2-f][1,2,4]triazolo[4,3-a][1,4]diazepin-6-yl)-2-oxo-6,9,12,15-tetraoxa-3-azaheptadecan-17-yl)carbamate) LC-MS: MS (ES⁺): RT=0.963 min, m/z=719.5 [M+H⁺].

### Part II-General Procedure for the Preparation of Compounds (0-4)PEG-BRD4

To a solution of compound 1a-e (0.9 mmol, 1.0 equiv) in dioxane (15 mL) was added HCl/dioxane (4 M, 5 mL, 21.7 equiv). The mixture was stirred at 25° C. for 2 h and then concentrated to afford compound (0-4)PEG-BRD4 (HCl salt) as a yellow solid.

### Physical Characterization Data for Compounds (0-4)PEG-BRD4:

Compound 0PEG-BRD4 ((S)-N-(2-aminoethyl)-2-(4-(4-chlorophenyl)-2,3,9-trimethyl-6H-thieno[3,2-f][1,2,4]triazolo[4,3-a][1,4]diazepin-6-yl)acetamide) LC-MS: MS (ES⁺): RT=0.702 min, m/z=443.2 [M+H⁺]; ¹H NMR (CD₃OD, 400 MHz) δ 7.7-7.9 (m, 1H), 7.5-7.7 (m, 3H), 4.9-5.0 (m, 1H), 3.4-3.6 (m, 3H), 3.0-3.2 (m, 3H), 2.91 (s, 2H), 2.5-2.35 (m, 4H), 1.7-2.0 (m, 3H).
Compound 1PEG-BRD4 ((S)-N-(2-(2-aminoethoxy)ethyl)-2-(4-(4-chlorophenyl)-2,3,9-trimethyl-6H-thieno[3,2-f][1,2,4]triazolo[4,3-a][1,4]diazepin-6-yl)acetamide) LC-MS: MS (ES⁺): RT=0.710 min, m/z=487.2 [M+H⁺]; ¹H NMR (CD₃OD, 400 MHz) δ 7.7-7.9 (m, 1H), 7.5-7.6 (m, 3H), 4.9-5.0 (m, 1H), 3.4-3.8 (m, 7H), 3.0-3.3 (m, 3H), 2.91 (s, 2H), 2.3-2.6 (m, 4H), 1.7-2.0 (m, 3H).
Compound 2PEG-BRD4 ((S)-N-(2-(2-(2-aminoethoxy)ethoxy)ethyl)-2-(4-(4-chlorophenyl)-2,3,9-trimethyl-6H-thieno[3,2-f][1,2,4]triazolo[4,3-a][1,4]diazepin-6-yl)acetamide) LC-MS: MS (ES⁺): RT=0.721 min, m/z=531.1 [M+H⁺]; ¹H NMR (CD₃OD, 400 MHz) δ 7.7-7.9 (m, 1H), 7.5-7.6 (m, 3H), 4.9-4.9 (m, 1H), 3.4-3.8 (m, 11H), 3.14 (brd, 3H), 2.90 (s, 2H), 2.3-2.6 (m, 4H), 1.7-2.0 (m, 3H).
Compound 3PEG-BRD4 ((S)-N-(2-(2-(2-(2-aminoethoxy)ethoxy)ethoxy)ethyl)-2-(4-(4-chlorophenyl)-2,3,9-trimethyl-6H-thieno[3,2-f][1,2,4]triazolo[4,3-a][1,4]diazepin-6-yl)acetamide) LC-MS: MS (ES⁺): RT=0.734 min, m/z=575.1 [M+H⁺]; ¹H NMR (CD₃OD, 400 MHz) δ 7.8-7.9 (m, 1H), 7.5-7.7 (m, 3H), 4.9-5.0 (m, 1H), 3.4-3.8 (m, 15H), 3.16 (m, 3H), 2.94 (s, 2H), 2.3-2.6 (m, 4H), 1.7-2.0 (m, 3H).
Compound 4PEG-BRD4 ((S)-N-(14-amino-3,6,9,12-tetraoxatetradecyl)-2-(4-(4-chlorophenyl)-2,3,9-trimethyl-6H-thieno[3,2-f][1,2,4]triazolo[4,3-a][1,4]diazepin-6-yl)acetamide) LC-MS: MS (ES⁺): RT=0.731 min, m/z=619.1 [M+H⁺]; ¹H NMR (CD₃OD, 400 MHz) δ 7.7-7.9 (m, 1H), 7.5-7.6 (m, 3H), 5.0-5.0 (m, 1H), 3.4-3.8 (m, 19H), 3.0-3.2 (m, 3H), 2.94 (s, 2H), 2.3-2.6 (m, 4H), 1.7-2.0 (m, 3H).

### Example 23: Preparation of Product Amide Compounds from Carboxylic Acid-Containing Protein Phosphatase Ligands and Amine-Containing Linker-Target Protein Ligands

The product amide compounds in Table 7 below were prepared from carboxylic acid-containing protein phosphatase ligands and amine-containing linker-target protein ligands according to the general synthetic procedures described in Example 11. Chemical structures for abbreviations used in the description of product amide compounds in Table 7 are provided in Tables 2 and 8.

TABLE 7
Exemplary Product Amide Compounds

| | | Protein | Linker-Target | | | | |
|---|---|---|---|---|---|---|---|
| | | Phosphatase | Protein | | | | |
| | | Ligand | Ligand Used to | | | | |
| Com- | | Used to Prepare | Prepare Product | | LC/MS | | |
| pound | Product Amide | Product Amide | Amide | | MH + | | |
| No. | Compound | Compound | Compound | MW | (obs) | RT | Method |
| I-219 | (Ac-RVSF)-(0PEG-BRD4) | Ac-RVSF | 0PEG-BRD4 | 975 | 974 | 2.23 | 10 |
| I-220 | (Ac-RVSF)-(1PEG-BRD4) | Ac-RVSF | 1PEG-BRD4 | 1019 | 1021 | 2.23 | 10 |
| I-221 | (Ac-RVSF)-(2PEG-BRD4) | Ac-RVSF | 2PEG-BRD4 | 1063 | 1065 | 2.25 | 10 |
| I-222 | (Ac-RVSF)-(3PEG-BRD4) | Ac-RVSF | 3PEG-BRD4 | 1107 | 1110 | 2.09 | 10 |
| I-223 | (Ac-RVSF)-(4PEG-BRD4) | Ac-RVSF | 4PEG-BRD4 | 1151 | 1153 | 2.26 | 10 |
| I-224 | (Oct-RVSF)-(0PEG- | Oct-RVSF | 0PEG-BRD4 | 1059 | 1062 | 2.56 | 10 |
| | BRD4) | | | | | | |
| I-225 | (Oct-RVSF)-(1PEG- | Oct-RVSF | 1PEG-BRD4 | 1103 | 1102 | 2.56 | 10 |
| | BRD4) | | | | | | |
| I-226 | (Oct-RVSF)-(2PEG- | Oct-RVSF | 2PEG-BRD4 | 1147 | 1149 | 2.58 | 10 |
| | BRD4) | | | | | | |
| I-227 | (Oct-RVSF)-(3PEG- | Oct-RVSF | 3PEG-BRD4 | 1191 | 1190 | 2.57 | 10 |
| | BRD4) | | | | | | |
| I-228 | (Oct-RVSF)-(4PEG- | Oct-RVSF | 4PEG-BRD4 | 1235 | 1235 | 2.57 | 10 |
| | BRD4) | | | | | | |
| I-229 | (H₂N-RVSF)-(0PEG-BRD4) | H₂N-RVSF | 0PEG-BRD4 | 933 | 932 | 2.1 | 10 |
| I-230 | (H₂N-RVSF)-(1PEG-BRD4) | H₂N-RVSF | 1PEG-BRD4 | 977 | 976 | 2.11 | 10 |
| I-231 | (H₂N-RVSF)-(2PEG-BRD4) | H₂N-RVSF | 2PEG-BRD4 | 1021 | 1020 | 2.14 | 10 |
| I-232 | (H₂N-RVSF)-(3PEG-BRD4) | H₂N-RVSF | 3PEG-BRD4 | 1065 | 1066 | 2.16 | 10 |
| I-233 | (H₂N-RVSF)-(4PEG-BRD4) | H₂N-RVSF | 4PEG-BRD4 | 1109 | 1108 | 2.17 | 10 |
| I-234 | (SMAP-Direct)-(0PEG-BRD4) | SMAP-Direct | 0PEG-BRD4 | 905 | 907 | 2.62 | 25 |
| I-235 | (SMAP-Direct)-(1PEG-BRD4) | SMAP-Direct | 1PEG-BRD4 | 950 | 950 | 2.63 | 25 |
| I-236 | (SMAP-Direct)-(2PEG-BRD4) | SMAP-Direct | 2PEG-BRD4 | 994 | 993 | 2.62 | 25 |
| I-237 | (SMAP-Direct)-(3PEG-BRD4) | SMAP-Direct | 3PEG-BRD4 | 1038 | 1037 | 2.62 | 25 |
| I-238 | (SMAP-Direct)-(4PEG-BRD4) | SMAP-Direct | 4PEG-BRD4 | 1082 | 1083 | 2.62 | 25 |
| I-239 | (SMAP-4DiF)-(0PEG-BRD4) | SMAP-4DiF | 0PEG-BRD4 | 971 | 972 | 2.73 | 25 |
| I-240 | (SMAP-4DiF)-(1PEG-BRD4) | SMAP-4DiF | 1PEG-BRD4 | 1016 | 1015 | 2.75 | 25 |
| I-241 | (SMAP-4DiF)-(2PEG-BRD4) | SMAP-4DiF | 2PEG-BRD4 | 1060 | 1059 | 2.74 | 25 |
| I-242 | (SMAP-4DiF)-(3PEG-BRD4) | SMAP-4DiF | 3PEG-BRD4 | 1104 | 1103 | 2.74 | 25 |
| I-243 | (SMAP-4DiF)-(4PEG-BRD4) | SMAP-4DiF | 4PEG-BRD4 | 1148 | 1148 | 2.74 | 25 |
| I-244 | (JNS 1-40)-(0PEG-BRD4) | JNS 1-40 | 0PEG-BRD4 | 817 | 816 | 2.69 | 10 |
| I-245 | (JNS 1-40)-(1PEG- | JNS 1-40 | 1PEG-BRD4 | 861 | 860 | 2.71 | 10 |
| | BRD4) | | | | | | |
| I-246 | (JNS 1-40)-(2PEG- | JNS 1-40 | 2PEG-BRD4 | 905 | 904 | 2.72 | 10 |
| | BRD4) | | | | | | |
| I-247 | (JNS 1-40)-(3PEG- | JNS 1-40 | 3PEG-BRD4 | 949 | 950 | 2.72 | 10 |
| | BRD4) | | | | | | |
| I-248 | (JNS 1-40)-(4PEG- | JNS 1-40 | 4PEG-BRD4 | 993 | 992 | 2.74 | 10 |
| | BRD4) | | | | | | |
| I-249 | (SMAP-3DiF)- | SMAP-3DiF | 0PEG-BRD4 | 971 | 971 | 2.75 | 25 |
| | (0PEG-BRD4) | | | | | | |
| I-250 | (SMAP-3DiF)- | SMAP-3DiF | 1PEG-BRD4 | 1016 | 1015 | 2.76 | 25 |
| | (1PEG-BRD4) | | | | | | |
| I-251 | (SMAP-3DiF)- | SMAP-3DiF | 2PEG-BRD4 | 1060 | 1062 | 2.77 | 25 |
| | (2PEG-BRD4) | | | | | | |
| I-252 | (SMAP-3DiF)- | SMAP-3DiF | 3PEG-BRD4 | 1104 | 1105 | 2.77 | 25 |
| | (3PEG-BRD4) | | | | | | |
| I-253 | (SMAP-3DiF)- | SMAP-3DiF | 4PEG-BRD4 | 1148 | 574 | 2.77 | 25 |
| | (4PEG-BRD4) | | | | [M/2 + H] | | |

TABLE 8
Chemical Structures Corresponding to Abbreviations Used in the Description of the Product
Amide Compounds

| Abbreviation | Chemical Structure |
|---|---|
| -(0PEG-BRD4) | 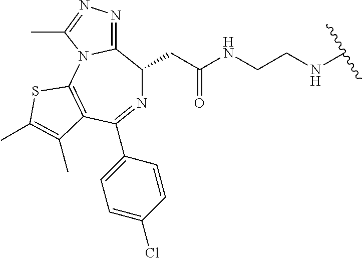 [SMILES: *NCCNC(=O)C[C@@H]1N=C(c2ccc(Cl)cc2)c2c(sc(C)c2C)-n2c(C)nnc21] |
| -(1PEG-BRD4) | 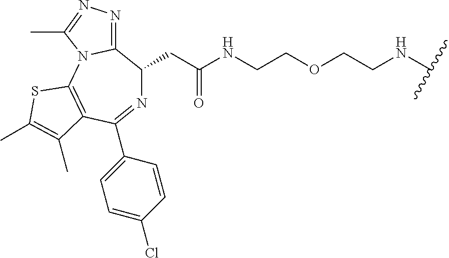 [SMILES: *NCCOCCNC(=O)C[C@@H]1N=C(c2ccc(Cl)cc2)c2c(sc(C)c2C)-n2c(C)nnc21] |
| -(2PEG-BRD4) | 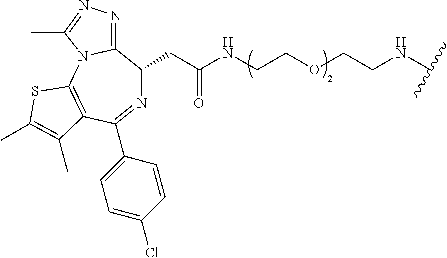 |
| -(3PEG-BRD4) | 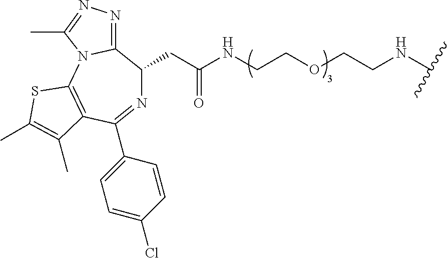 |
| -(4PEG-BRD4) | 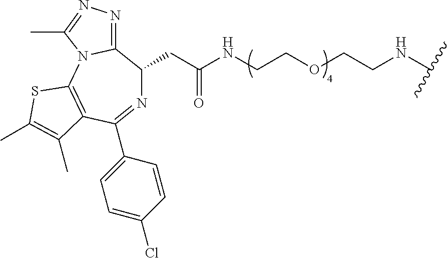 |

### Example 24: Preparation of Compounds Ac-CRVS(MeF)—OH, Ac-CRVS(MeF)-NMe, Ac-CRVSA-OH, and Ac-CRVSA-NMe (Protein Phosphatase Ligands)

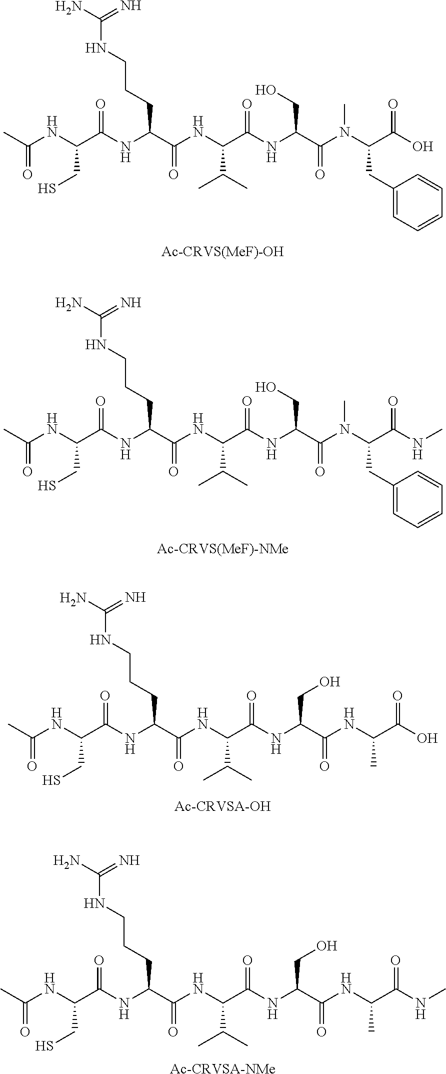

### Part I-General Procedure for Solid-Phase Peptide Synthesis: Protected Compound Ac-CRVS(MeF)—OH

The title compounds were synthesized via solid phase peptide synthesis and resin cleavage, according to the general synthetic procedures described in Example 2, above, and depicted in the scheme below for representative compound Ac-CRVS(MeF)—OH.

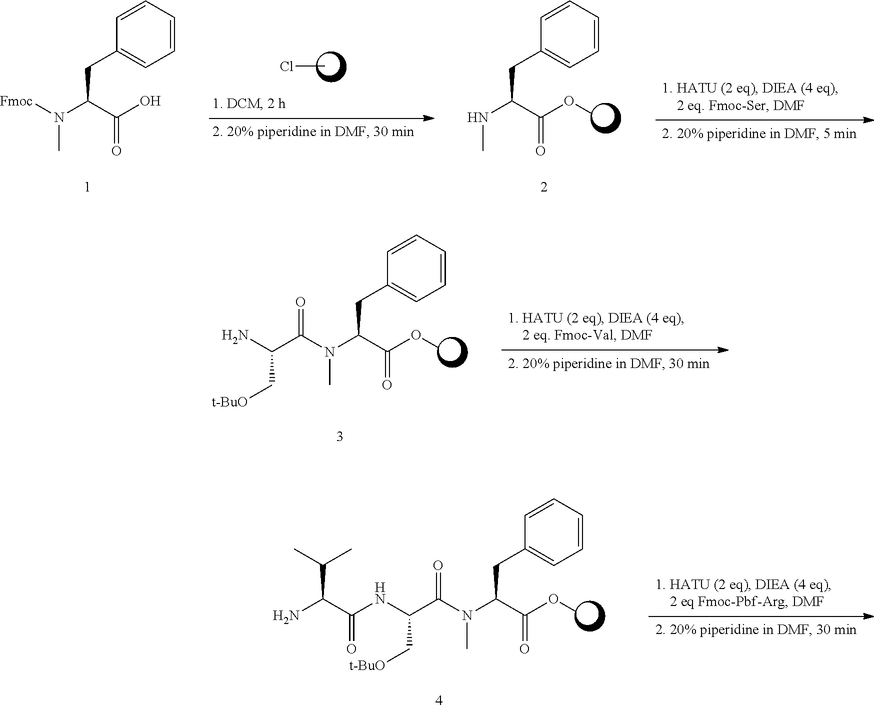

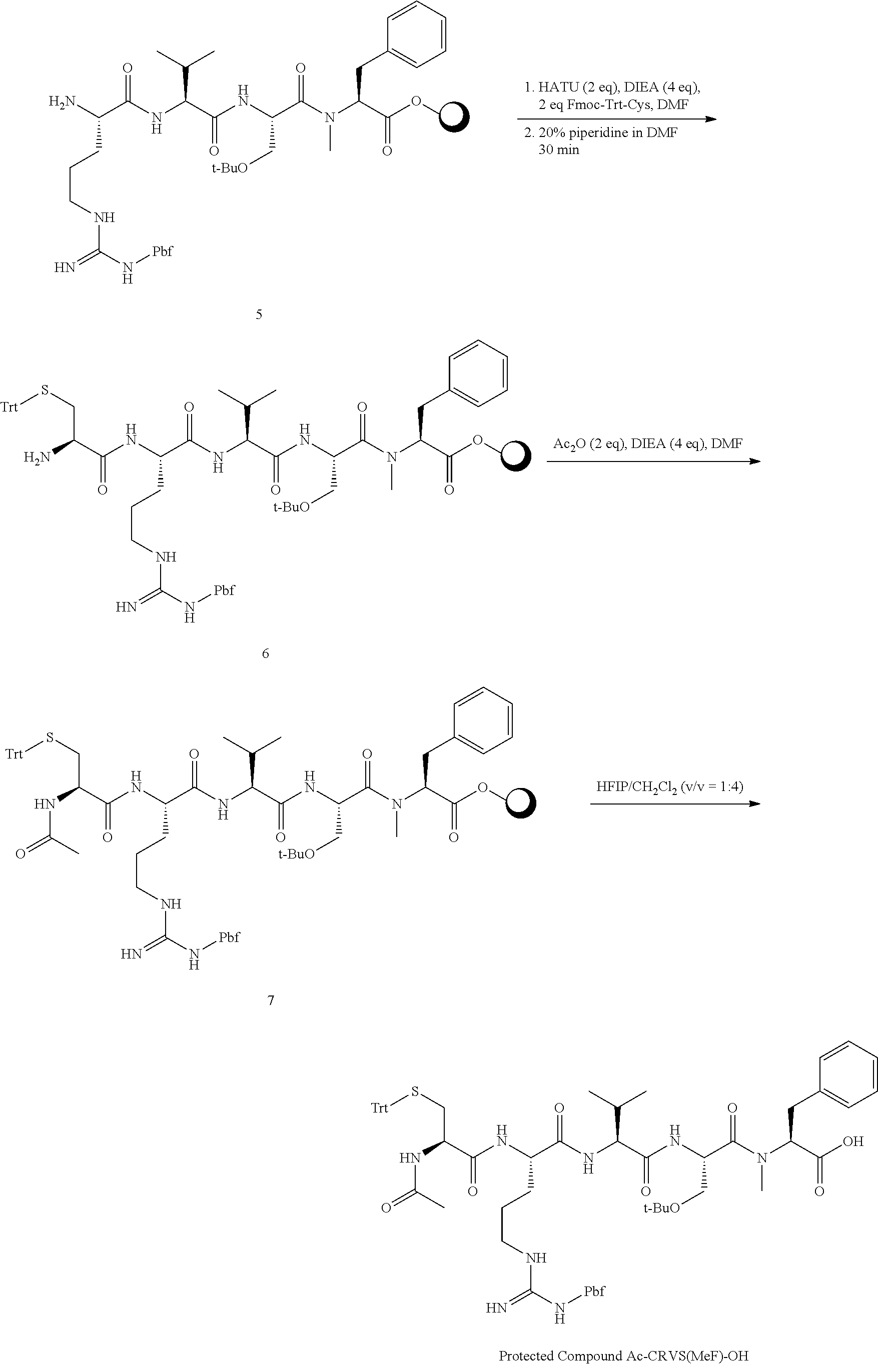

### Physical Characterization Data for Protected Compound AcCRVS(MeF)—OH:

(4R,7S,10S, 13S,16S)-4-acetamido-16-benzyl-13-(tert-butoxymethyl)-10-isopropyl-15-methyl-5,8,11,14-tetraoxo-7-(3-(3-((2,2,4,6,7-pentamethyl-2,3-dihydrobenzofuran-5-yl)sulfonyl)guanidino)propyl)-1,1,1-triphenyl-2-thia-6,9,12,15-tetraazaheptadecan-17-oic acid. 1HNMR (400 MHz, CD₃OD): δ 7.39-7.41 (m, 6H), 7.29-7.32 (m, 6H), 7.20-7.26 (m, 8H), 5.05-5.09 (m, 1H), 4.91-4.95 (m, 2H), 4.64-4.69 (m, 4H), 4.35-4.39 (m, 1H), 4.14-4.16 (m, 1H), 3.53-3.58 (m, 1H), 3.42-3.45 (m, 1H), 3.37 (s, 1H), 3.04-3.16 (m, 4H), 3.00-3.04 (m, 4H), 2.99 (s, 1H), 2.88-2.89 (m, 3H), 2.58 (s, 3H), 2.52 (s, 3H), 2.07 (s, 3H), 1.94 (s, 3H), 1.76-1.82 (m, 1H), 1.61-1.66 (m, 1H), 1.46 (s, 3H), 1.16 (s, 9H), 0.79-0.82 (m, 6H). LC-MS: MS (ES⁺): RT=1.050 min, m/z=1217.7 [M+H⁺].

### Part II-Preparation of Compound AcCRVS(MeF)-NMe

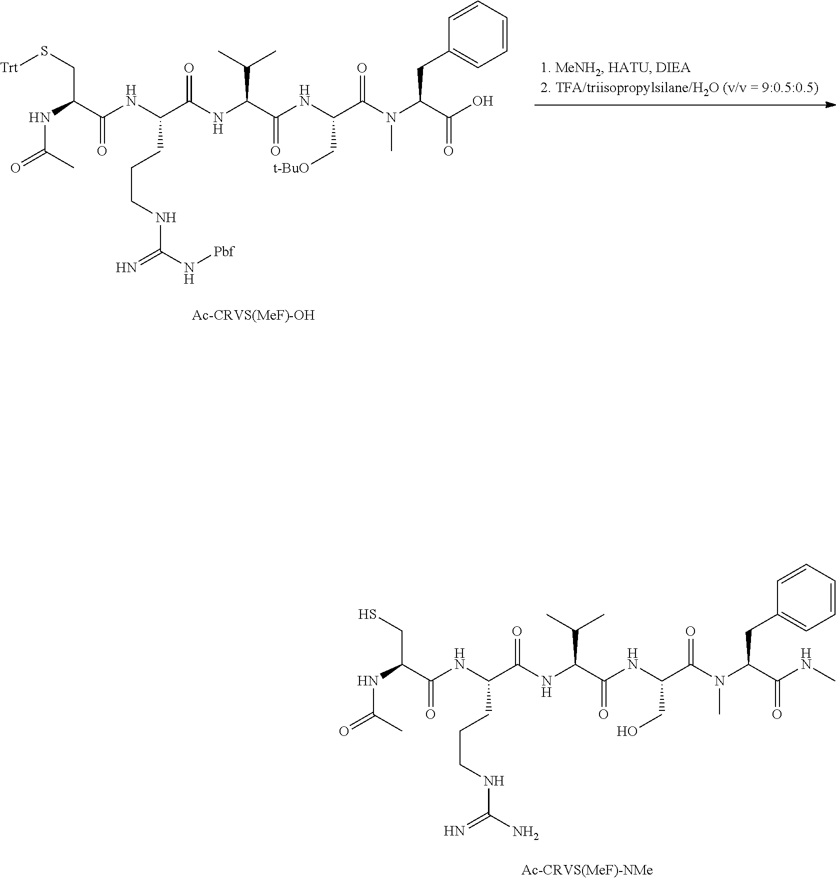

To a solution of protected compound Ac-CRVS(MeF)—OH (1.7 g, 1.4 mmol, 1.0 equiv) and MeNH₂ (283 mg, 4.2 mmol, 3.0 equiv, HCl salt) in DMF (10 mL) were added HATU (637 mg, 1.7 mmol, 1.2 equiv) and DIEA (541 mg, 4.2 mmol, 3.0 equiv). The mixture was stirred at 25° C. for 16 h and then 50 mL water was added to the mixture to quench the reaction. The mixture was extracted with ethyl acetate (3×100 mL). The combined organic layers were concentrated to afford the protected compound AcCRVS(MeF)-NMe which was used directly for the next step. LC-MS: MS (ES⁺): RT=1.195 min, m/z=1130.5 [M+H⁺].
A mixture of protected compound AcCRVS(MeF)-NMe (1.7 g, 138 mmol), triisopropylsilane (0.5 mL) and water (0.5 mL) in TFA (9 mL) was stirred at 20° C. for 1.5 h. The mixture was added dropwise to 2-methoxy-2-methylpropane (20 mL), and the resulting suspension was filtered. The filter cake was washed with 2-methoxy-2-methylpropane (3×5 mL). The solid was dissolved in DMSO (2 mL) and purified by prep-HPLC (column: 3_Phenomenex Luna C18 75*30 mm*3 um; mobile phase: [water(0.05% HCl)-ACN]; B %: 14%-34%, 6.5 min) to afford compound AcCRVS(MeF)-NMe ((S)-2-((R)-2-acetamido-3-mercaptopropanamido)-5-guanidino-N-((S)-1-(((S)-3-hydroxy-1-(methyl((S)-1-(methylamino)-1-oxo-3-phenylpropan-2-yl)amino)-1-oxopropan-2-yl)amino)-3-methyl-1-oxobutan-2-yl)pentanamide). 1HNMR (400 MHz, DMSO-d₆): δ 8.37-8.39 (m, 1H), 8.31-8.33 (m, 1H), 8.08-8.15 (m, 1H), 7.60-7.62 (m, 1H), 7.58-7.60 (m, 2H), 7.14-7.28 (m, 5H), 5.04-5.09 (m, 1H), 4.72-4.75 (m, 1H), 4.53-4.55 (m, 1H), 4.31-4.33 (m, 1H), 4.16-4.19 (m, 1H), 3.05-3.23 (m, 5H), 2.70-2.93 (m, 4H), 2.50-2.64 (m, 5H), 1.38-1.63 (m, 8H), 0.71-0.81 (m, 6H). LC-MS: MS (ES⁺): RT=0.664 min, m/z=679.6 [M+H⁺].

### Part III-Preparation of Compounds AcCRVSA-OH and AcCRVSA-NMe

Compounds AcCRVSA-OH and AcCRVSA-NMe were prepared by analogy to the procedures described in Part I and II.

### Physical Characterization Data for Compounds AcCRVSA-OH and AcCRVSA-NMe:

Compound AcCRVSA-OH ((2S,5S,8S,11S,14R)-11-(3-guanidinopropyl)-5-(hydroxymethyl)-8-isopropyl-14-(mercaptomethyl)-2-methyl-4,7,10,13,16-pentaoxo-3,6,9,12,15-pentaazaheptadecan-1-oic acid)¹HNMR (400 MHz, DMSO-d₆+D₂O): δ 4.18-4.37 (m, 5H), 3.55-3.60 (m, 2H), 3.06-3.10 (m, 2H), 2.65-2.75 (m, 2H), 1.95-2.05 (m, 1H), 1.87 (s, 3H), 1.40-1.69 (m, 4H), 1.25-1.27 (m, 3H), 0.80-0.86 (m, 6H). LC-MS: MS (ES⁺): RT=2.068 min, m/z=577.2 [M+H⁺].
Compound AcCRVSA-NMe ((S)-2-((R)-2-acetamido-3-mercaptopropanamido)-5-guanidino-N-((S)-1-(((S)-3-hydroxy-1-(((S)-1-(methylamino)-1-oxopropan-2-yl)amino)-1-oxopropan-2-yl)amino)-3-methyl-1-oxobutan-2-yl)pentanamide)¹HNMR (400 MHz, DMSO-d₆+D₂O): δ 4.17-4.38 (m, 5H), 3.60-3.62 (m, 1H), 3.07-3.10 (m, 2H), 2.65-2.75 (m, 2H), 2.50-2.55 (m, 3H), 2.33-2.35 (m, 1H), 1.95-2.05 (m, 1H), 1.87 (s, 3H), 1.40-1.69 (m, 4H), 1.19-1.22 (m, 3H), 0.80-0.86 (m, 6H). LC-MS: MS (ES⁺): RT=2.054 min, m/z=590.3 [M+H⁺].

### Example 25: Preparation of Compound Iodo-2PEG-AKTallo (Linker-Target Protein Ligand)

The title compound was prepared according to the procedure below, as described and depicted in the scheme. Preparation of compound 2PEG-AKTallo is described in Example 8.

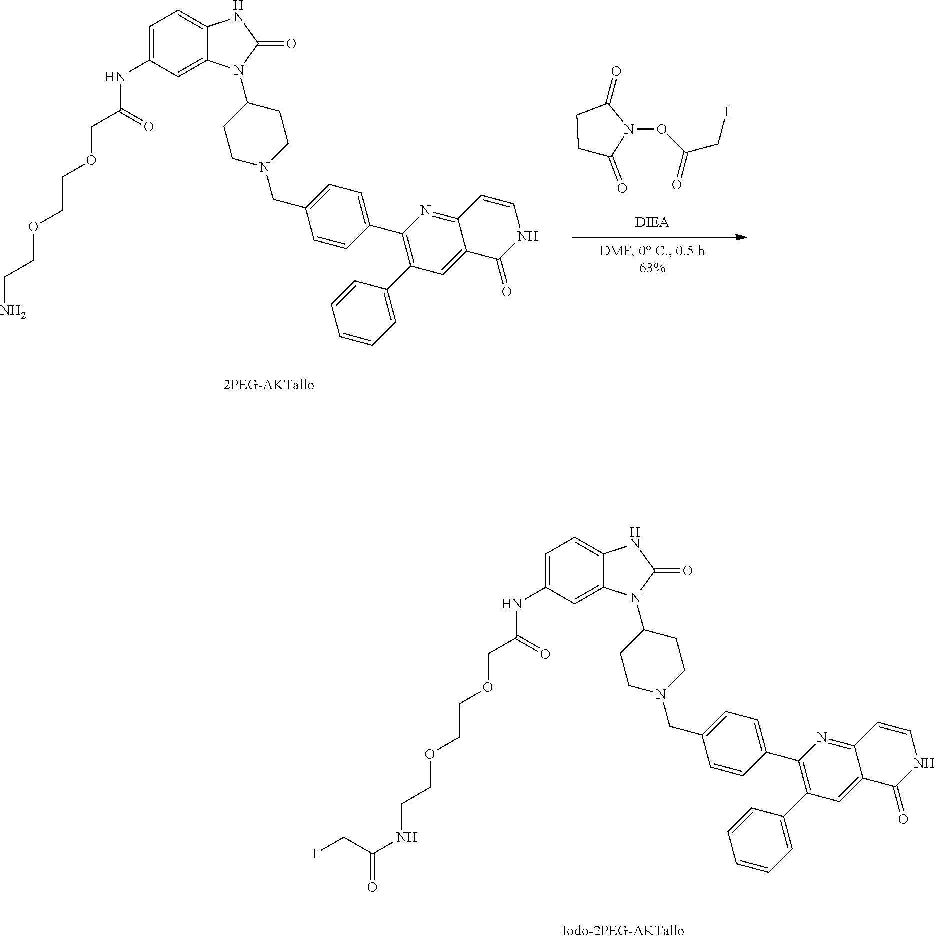 [SMILES: NCCOCCOCC(=O)Nc1ccc2[nH]c(=O)n(C3CCN(Cc4ccc(-c5nc6cc[nH]c(=O)c6cc5-c5ccccc5)cc4)CC3)c2c1 ; O=C(CI)ON1C(=O)CCC1=O ; O=C(CI)NCCOCCOCC(=O)Nc1ccc2[nH]c(=O)n(C3CCN(Cc4ccc(-c5nc6cc[nH]c(=O)c6cc5-c5ccccc5)cc4)CC3)c2c1]

### Part I-Preparation of Compound Iodo-2PEG-AKTallo

To a solution of 2PEG-AKTallo (0.5 g, 624 μmol, 1.0 equiv, TFA salt) and DIEA (161 mg, 1.25 mmol, 217 μL, 2.0 equiv) in DMF (5 mL) was added (2,5-dioxopyrrolidin-1-yl) 2-iodoacetate (194.14 mg, 686 μmol, 1.1 equiv) at 0° C. The mixture was stirred at 0° C. for 0.5 h. To the reaction mixture at 0° C. was added TFA to pH=5. The solution was purified by prep-HPLC on a 150*50 mm*3 um Unisil 3-100 C18 column with TFA modified water to afford Iodo-2PEG-AKTallo (0.38 g, 392 μmol, 63% yield, TFA salt) as a yellow solid. 2-[2-[2-[(2-iodoacetyl)amino]ethoxy]ethoxy]-N-[2-oxo-3-[1-[[4-(5-oxo-3-phenyl-6H-1,6-naphthyridin-2-yl)phenyl]methyl]-4-piperidyl]-1H-benzimidazol-5-yl]acetamide. ¹H NMR (400 MHz, CD₃OD): δ 8.64 (s, 1H), 7.68 (s, 1H), 7.53-7.56 (m, 5H), 7.31-7.32 (m, 3H), 7.26 (m, 2H), 7.06 (m, 1H), 7.0-7.1 (m, 1H), 6.8-6.9 (m, 1H, J=7.6 Hz), 4.53-4.55 (m, 1H), 4.41 (s, 2H), 4.17 (s, 2H), 3.74-3.79 (m, 2H), 3.70-3.74 (m, 1H), 3.60-3.66 (m, 6H), 3.38-3.39 (m, 2H), 3.31 (m, 1H), 2.80-2.86 (m, 2H), 2.11-2.14 (m, 2H); LC-MS: MS (ES⁺): RT=0.782 min, m/z=856.2 [M+H⁺].

### Example 26: Preparation of Thioether Compounds from Thiol-Containing Protein Phosphatase Ligands and Halo-Containing Linker-Target Protein Ligands

The thioether compounds in Table 9 below were prepared from thiol-containing protein phosphatase ligands and halo-containing linker-target protein ligands according to the general synthetic procedures below, as described and depicted in the scheme.

### Part I-General Procedure for Thioether Formation

To a mixture of halo-containing linker-target protein ligand (˜60 μmol, 1.0 equiv, TFA salt) in a HEPES buffer (1 M, 6.20 mL, pH=8) was added thiol-containing protein phosphatase ligand (˜1.0 equiv, HCl salt) in DMSO (1 mL) at 0° C. The suspension was stirred at 20° C. for 12 h. The mixture was acidified by adding TFA to pH=4-5 at 0° C. At this point, the mixture became a clear solution, and was directly purified by prep-HPLC to afford the product thioether compound.
The following scheme depicts the thioether formation procedure for thiol-containing protein phosphatase ligand Ac-RVSA-OH and halo-containing linker-target protein ligand Iodo-2PEG-AKTallo to afford Compound I-255.

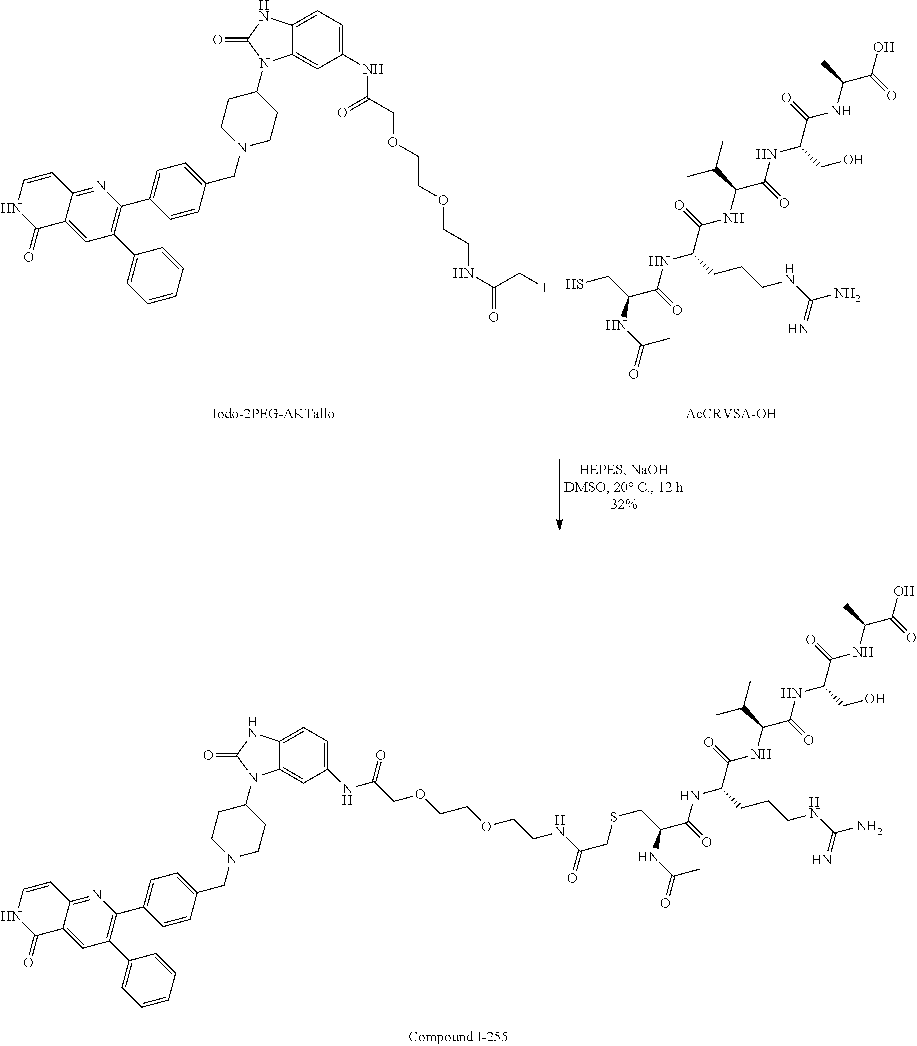

Physical characterization data for Compound I-255:
Compound I-255 (29 mg, 20 μmol, 32% yield, 90.39% purity, HCl salt) was isolated as a yellow solid following purification by prep-HPLC (column: 3_Phenomenex Luna C18 75*30 mm*3 um; mobile phase: [water(0.05% HCl)-ACN]; B %: 12%-32%; 6.5 min). (2S)-2-[[(2S)-2-[[(2S)-2-[[(2S)-2-[[(2R)-2-acetamido-3-[2-oxo-2-[2-[2-[2-oxo-2-[[2-oxo-3-[1-[[4-(5-oxo-3-phenyl-6H-1,6-naphthyridin-2-yl)phenyl]methyl]-4-piperidyl]-1H-benzimidazol-5-yl]amino]ethoxy]ethoxy]ethylamino]ethyl]sulfanyl-propanoyl]amino]-5-guanidino-pentanoyl]amino]-3-methyl-butanoyl]amino]-3-hydroxy-propanoyl]amino]propanoic acid. ¹H NMR (400 MHz, CD₃OD): δ 9.17 (s, 1H), 7.90-7.92 (d, 1H, J=7.2 Hz), 7.79-7.77 (m, 2H), 7.68-7.71 (m, 3H), 7.37-7.39 (m, 3H), 7.31-7.32 (m, 3H), 7.04-7.06 (m, 2H), 4.51 (m, 1H), 4.46 (m, 6H), 4.19-4.22 (m, 3H), 3.77-3.80 (m, 6H), 3.65 (m, 4H), 3.44 (m, 2H), 3.32 (m, 2H), 3.30-3.31 (m, 2H), 3.21-3.25 (m, 2H), 2.86-2.88 (m, 4H), 2.13-2.0.9 (m, 3H), 1.99 (m, 4H), 1.67 (m, 3H), 1.40-1.41 (m, 2H, J=7.2 Hz), 0.93-0.95 (m, 6H); LC-MS: MS (ES⁺): RT=1.854 min, m/z=1304.6 [M+H⁺].
Part II-Exemplary product thioether compounds prepared according to the general Procedure
The product thioether compounds described in Table 9 below were prepared from thiol-containing protein phosphatase ligands and halo-containing linker-target protein ligands according to the procedure described in Part I. Chemical structures for abbreviations used in the description of product thioether compounds in Table 9 are provided in Table 10 below.

TABLE 9
Exemplary Product Thioether Compounds

| | | Protein | Linker-Target | | | | |
|---|---|---|---|---|---|---|---|
| | | Phosphatase | Protein | | | | |
| | | Ligand | Ligand Used to | | | | |
| Com- | | Used to Prepare | Prepare Product | | LC/MS | | |
| pound | Product Thioeter | Product Thioeter | Thioeter | | MH + | | |
| No. | Compound | Compound | Compound | MW | (obs) | RT | Method |
| I-254 | (AcCRVS(MeF)- | AcCRVS(MeF)- | Iodo-2PEG- | 1408 | 1407 | 2.03 | 10 |
| | NMe)-(Iodo-2PEG- | NMe | AKTallo | | | | |
| | AKTallo) | | | | | | |
| I-255 | (AcCRVSA-OH)- | AcCRVSA-OH | Iodo-2PEG- | 1305 | 1305 | 1.85 | 10 |
| | (Iodo-2PEG-AKTallo) | | AKTallo | | | | |
| I-256 | (AcCRVSA-NMe)- | AcCRVSA-NMe | Iodo-2PEG- | 1318 | 1317 | 1.85 | 10 |
| | (Iodo-2PEG-AKTallo) | | AKTallo | | | | |
| I-257 | (AcCRVS(MeF)- | AcCRVS(MeF)- | Iodo-3PEG- | 1424 | 1425 | 1.90 | 10 |
| | NMe)-(Iodo-3PEG- | NMe | AKTcomp | | | | |
| | AKTcomp) | | | | | | |
| I-258 | (AcCRVSA-NMe)- | AcCRVSA-NMe | (Iodo-3PEG- | 1334 | 1334 | 1.72 | 10 |
| | Iodo-3PEG- | | AKTcomp) | | | | |
| | AKTcomp | | | | | | |

TABLE 10
Chemical Structures for Abbreviations Used in the Description of Product Thioether Compounds

| Abbreviation | Chemical Structure |
|---|---|
| (AcCRVS(MeF)- OH)- | 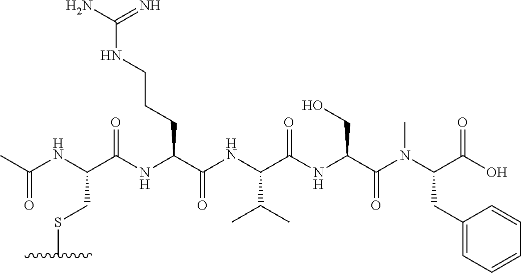 [SMILES: *SC[C@H](NC(C)=O)C(=O)N[C@@H](CCCNC(=N)N)C(=O)N[C@H](C(=O)N[C@@H](CO)C(=O)N(C)[C@@H](Cc1ccccc1)C(=O)O)C(C)C] |
| (AcCRVS(MeF)- NMe)- | 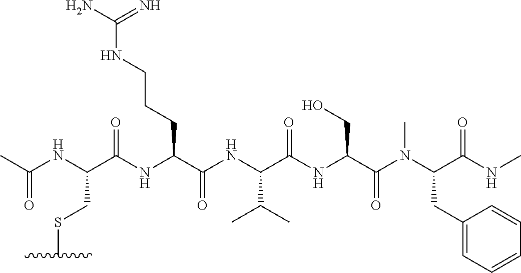 [SMILES: *SC[C@H](NC(C)=O)C(=O)N[C@@H](CCCNC(=N)N)C(=O)N[C@H](C(=O)N[C@@H](CO)C(=O)N(C)[C@@H](Cc1ccccc1)C(=O)NC)C(C)C] |
| (AcCRVSA-OH)- | 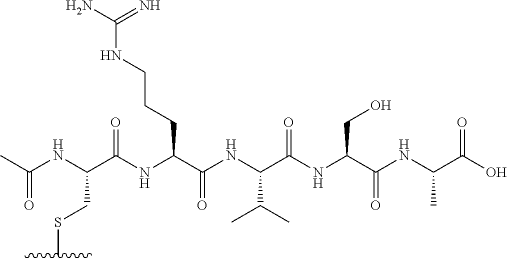 [SMILES: *SC[C@H](NC(C)=O)C(=O)N[C@@H](CCCNC(=N)N)C(=O)N[C@H](C(=O)N[C@@H](CO)C(=O)N[C@@H](C)C(=O)O)C(C)C] |
| (AcCRVSA- NMe)- | 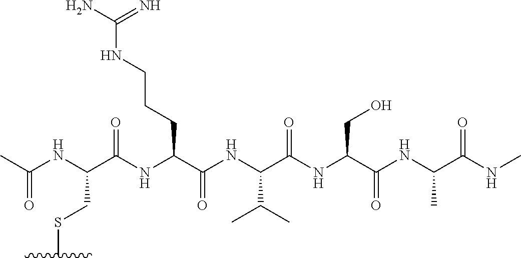 [SMILES: *SC[C@H](NC(C)=O)C(=O)N[C@@H](CCCNC(=N)N)C(=O)N[C@H](C(=O)N[C@@H](CO)C(=O)N[C@@H](C)C(=O)NC)C(C)C] |
| -(Iodo-2PEG- AKTallo) | 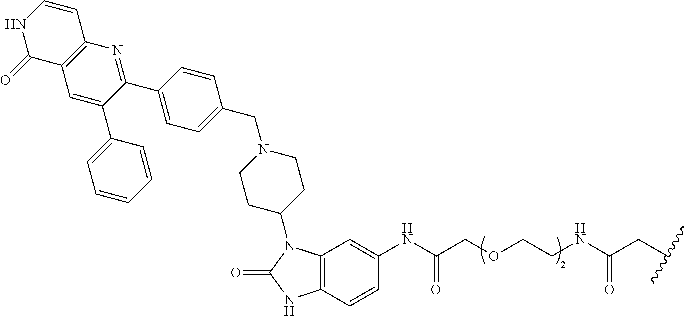 |
| -(Iodo-3PEG- AKTcomp) | 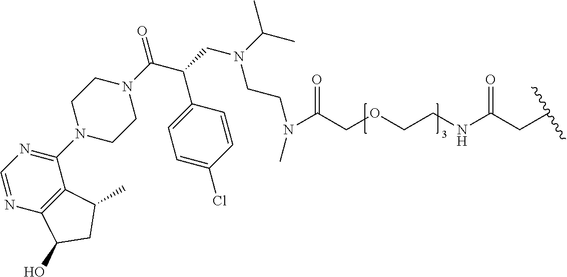 |

### Example 27: Additional Thioether Compounds for Preparation from Thiol-Containing Protein Phosphatase Ligands and Halo-Containing Linker-Target Protein Ligands

The thioether compounds in Table 11 below may be prepared from thiol-containing protein phosphatase ligands and halo-containing linker-target protein ligands according to the general synthetic procedures described in Example 26. Chemical structures for abbreviations used in the description of product thioether compounds in Table 11 are provided in Table 10.

TABLE 11
Exemplary Product Thioether Compounds

| | | Protein Phosphatase | Linker-Target Protein |
|---|---|---|---|
| | | Ligand To Be Used for | Ligand To Be Used for |
| Compound | | Preparation of Product | Preparation of Product |
| No. | Product Thioether Compound | Thioether Compound | Thioether Compound |
| I-259 | (AcCRVS(MeF)-OH)-(Iodo-2PEG-AKTallo) | AcCRVS(MeF)-OH | Iodo-2PEG-AKTallo |
| I-260 | (AcCRVS(MeF)-OH)-(Iodo-3PEG-AKTcomp) | AcCRVS(MeF)-OH | Iodo-3PEG-AKTcomp |
| I-261 | (AcCRVSA-OH)-(Iodo-3PEG-AKTcomp) | AcCRVSA-OH | Iodo-3PEG-AKTcomp |

### Example 28: Preparation of Additional Product Amide Compounds from Carboxylic Acid-Containing Protein Phosphatase Ligands and Amine-Containing Linker-Target Protein Ligands

Product amide compounds in Table 12 were prepared from carboxylic acid-containing protein phosphatase ligands and amine-containing linker-target protein ligands according to the general synthetic procedures described in Examples 2-11. Chemical structures for abbreviations used in the description of product amide compounds in Table 12 are depicted in Tables 2 and 13 herein.

TABLE 12
Exemplary Product Amide Compounds

| | | Protein | Linker-Target | | | | |
|---|---|---|---|---|---|---|---|
| | | Phosphatase | Protein | | | | |
| | | Ligand | Ligand Used to | | | | |
| Com- | | Used to Prepare | Prepare Product | | LC/MS | | |
| pound | Product Amide | Product Amide | Amide | | MH + | | |
| No. | Compound | Compound | Compound | MW | (obs.) | RT | Method |
| I-262 | (Ac-KRVHF)-(0PEG- | Ac-KRVHF | 0PEG- | 990.63 | 990.5 | 1.88 | 10 |
| | Chloroalkane) | | Chloroalkane | | | | |
| I-263 | (Ac-KRVHF)-(1PEG- | Ac-KRVHF | 1PEG- | 1034.7 | 1034.4 | 1.91 | 10 |
| | Chloroalkane) | | Chloroalkane | | | | |
| I-264 | (Ac-KRVHF)-(2PEG- | Ac-KRVHF | 2PEG- | 1078.7 | 1078.5 | 1.94 | 10 |
| | Chloroalkane) | | Chloroalkane | | | | |
| I-265 | (Ac-KRVHF)-(3PEG- | Ac-KRVHF | 3PEG- | 1122.8 | 1122.5 | 1.96 | 10 |
| | Chloroalkane) | | Chloroalkane | | | | |
| I-266 | (Ac-KRVHF)-(4PEG- | Ac-KRVHF | 4PEG- | 1166.8 | 1166.6 | 1.98 | 10 |
| | Chloroalkane) | | Chloroalkane | | | | |
| | | | 0PEG- | | | | |
| I-267 | (Oct-KRVHF)- | Oct-KRVHF | | 1074.8 | 1074.6 | 2.46 | 01 |
| | (0PEG-Chloroalkane) | | Chloroalkane | | | | |
| I-268 | (Oct-KRVHF)- | Oct-KRVHF | 1PEG- | 1118.8 | 1118.6 | 2.48 | 01 |
| | (1PEG-Chloroalkane) | | Chloroalkane | | | | |
| I-269 | (Oct-KRVHF)- | Oct-KRVHF | 2PEG- | 1162.9 | 1163.6 | 2.48 | 01 |
| | (2PEG-Chloroalkane) | | Chloroalkane | | | | |
| I-270 | (Oct-KRVHF)- | Oct-KRVHF | 3PEG- | 1207 | 1207.7 | 2.50 | 01 |
| | (3PEG-Chloroalkane) | | Chloroalkane | | | | |
| I-271 | (Oct-KRVHF)- | Oct-KRVHF | 4PEG- | 1251 | 625.9 | 2.50 | 01 |
| | (4PEG-Chloroalkane) | | Chloroalkane | | [M/2 + H] | | |

TABLE 13
Chemical Structures for Abbreviations Used in the Description of Product Amide Compounds

| Abbreviation | Chemical Structure |
|---|---|
| (Ac-KRVHF)- | 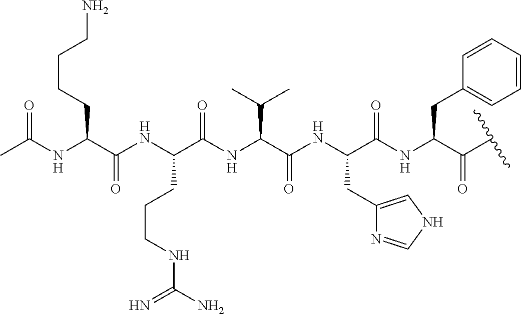 [SMILES: *C(=O)[C@H](Cc1ccccc1)NC(=O)[C@H](Cc1c[nH]cn1)NC(=O)[C@@H](NC(=O)[C@H](CCCNC(=N)N)NC(=O)[C@H](CCCCN)NC(C)=O)C(C)C] |
| (Oct-KRVHF)- | 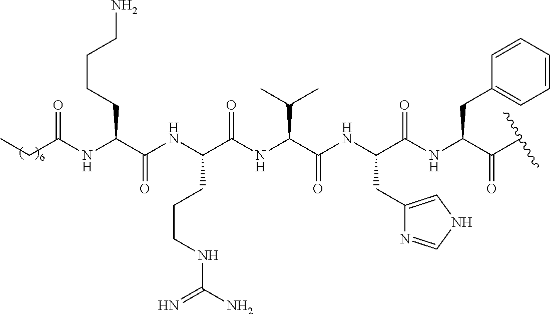 |

Example 29: Additional Product Amide Compounds for Preparation from Carboxylic Acid-Containing Protein Phosphatase Ligands and Amine-Containing Linker-Target Protein Ligands

Product amide compounds in Tables 14-16 may be prepared from carboxylic acid-containing protein phosphatase ligands and amine-containing linker-target protein ligands according to the general synthetic procedures described in Examples 2-11 and 22. Chemical structures for abbreviations used in the description of product amide compounds in Tables 14-16 are provided in Tables 2, 8, 13, and 17.

TABLE 14
Exemplary Product Amide Compounds

| | | Protein | |
|---|---|---|---|
| | | Phosphatase | |
| | | Ligand | Linker-Target |
| | | To Be | Protein Ligand |
| | | Used in | To Be Used in |
| | | Preparation | Preparation |
| Com- | | of Product | of Product |
| pound | | Amide | Amide |
| No. | Product Amide Compound | Compound | Compound |
| I-272 | (Ac-KRVHF)-(0PEG-AKTallo) | Ac-KRVHF | 0PEG-AKTallo |
| I-273 | (Ac-KRVHF)-(1PEG-AKTallo) | Ac-KRVHF | 1PEG-AKTallo |
| I-274 | (Ac-KRVHF)-(2PEG-AKTallo) | Ac-KRVHF | 2PEG-AKTallo |
| I-275 | (Ac-KRVHF)-(3PEG-AKTallo) | Ac-KRVHF | 3PEG-AKTallo |
| I-276 | (Ac-KRVHF)-(4PEG-AKTallo) | Ac-KRVHF | 4PEG-AKTallo |
| I-277 | (Oct-KRVHF)-(0PEG-AKTallo) | Oct-KRHF | 0PEG-AKTallo |
| I-278 | (Oct-KRVHF)-(1PEG-AKTallo) | Oct-KRHF | 1PEG-AKTallo |
| I-279 | (Oct-KRVHF)-(2PEG-AKTallo) | Oct-KRHF | 2PEG-AKTallo |
| I-280 | (Oct-KRVHF)-(3PEG-AKTallo) | Oct-KRVHF | 3PEG-AKTallo |
| I-281 | (Oct-KRVHF)-(4PEG-AKTallo) | Oct-KRHF | 4PEG-AKTallo |
| I-282 | (Ac-KRVHF)-(0PEG-AKTcomp) | Ac-KRVHF | 0PEG-AKTcomp |
| I-283 | (Ac-KRVHF)-(1PEG-AKTcomp) | Ac-KRVHF | 1PEG-AKTcomp |
| I-284 | (Ac-KRVHF)-(2PEG-AKTcomp) | Ac-KRVHF | 2PEG-AKTcomp |
| I-285 | (Ac-KRVHF)-(3PEG-AKTcomp) | Ac-KRVHF | 3PEG-AKTcomp |
| I-286 | (Ac-KRVHF)-(4PEG-AKTcomp) | Ac-KRVF | 4PEG-AKTcomp |
| I-287 | (Oct-KRVHF)-(0PEG-AKTcomp) | Oct-KRVHF | 0PEG-AKTcomp |
| I-288 | (Oct-KRVHF)-(1PEG-AKTcomp) | Oct-KRVHF | 1PEG-AKTcomp |
| I-289 | (Oct-KRVHF)-(2PEG-AKTcomp) | Oct-KRVHF | 2PEG-AKTcomp |
| I-290 | (Oct-KRVHF)-(3PEG-AKTcomp) | Oct-KRVHF | 3PEG-AKTcomp |
| I-291 | (Oct-KRVHF)-(4PEG-AKTcomp) | Oct-KRVHF | 4PEG-AKTcomp |
| I-292 | (Ac-KRVHF)-(0PEG-TBK1) | Ac-KRVHF | 0PEG-TBK1 |
| I-293 | (Ac-KRVHF)-(1PEG-TBK1) | Ac-KRVHF | 1PEG-TBK1 |
| I-294 | (Ac-KRVHF)-(2PEG-TBK1) | Ac-KRVHF | 2PEG-TBK1 |
| I-295 | (Ac-KRVHF)-(3PEG-TBK1) | Ac-KRVHF | 3PEG-TBK1 |
| I-296 | (Ac-KRVHF)-(4PEG-TBK1) | Ac-KRVHF | 4PEG-TBK1 |
| I-297 | (Oct-KRVHF)-(0PEG-TBK1) | Oct-KRVHF | 0PEG-TBK1 |
| I-298 | (Oct-KRVHF)-(1PEG-TBK1) | Oct-KRVHF | 1PEG-TBK1 |
| I-299 | (Oct-KRVHF)-(2PEG-TBK1) | Oct-KRVHF | 2PEG-TBK1 |
| I-300 | (Oct-KRVHF)-(3PEG-TBK1) | Oct-KRVHF | 3PEG-TBK1 |
| I-301 | (Oct-KRVHF)-(4PEG-TBK1) | Oct-KRVHF | 4PEG-TBK1 |
| I-302 | (Ac-KRVHF)-(0PEG-BRD4) | Ac-KRVHF | 0PEG-BRD4 |
| I-303 | (Ac-KRVHF)-(1PEG-BRD4) | Ac-KRVHF | 1PEG-BRD4 |
| I-304 | (Ac-KRVHF)-(2PEG-BRD4) | Ac-KRVHF | 2PEG-BRD4 |
| I-305 | (Ac-KRVHF)-(3PEG-BRD4) | Ac-KRVHF | 3PEG-BRD4 |
| I-306 | (Ac-KRVHF)-(4PEG-BRD4) | Ac-KRVHF | 4PEG-BRD4 |
| I-307 | (Oct-KRVHF)-(0PEG-BRD4) | Oct-KRVHF | 0PEG-BRD4 |
| I-308 | (Oct-KRVHF)-(1PEG-BRD4) | Oct-KRVHF | 1PEG-BRD4 |
| I-309 | (Oct-KRVHF)-(2PEG-BRD4) | Oct-KRVHF | 2PEG-BRD4 |
| I-310 | (Oct-KRVHF)-(3PEG-BRD4) | Oct-KRVHF | 3PEG-BRD4 |
| I-311 | (Oct-KRVHF)-(4PEG-BRD4) | Oct-KRVHF | 4PEG-BRD4 |

TABLE 15
Exemplary Product Amide Compounds

| | | Protein | |
|---|---|---|---|
| | | Phosphatase | |
| | | Ligand To Be | |
| | | Used in | |
| | | Preparation | Linker-Target Protein |
| | | of Product | Ligand To Be Used in |
| Compound | | Amide | Preparation of Product |
| No. | Product Amide Compound | Compound | Amide Compound |
| I-312 | (Ac-KRVSF)-(0PEG-Chloroalkane) | Ac-KRVSF | 0PEG-Chloroalkane |
| I-313 | (Ac-KRVSF)-(1PEG-Chloroalkane) | Ac-KRVSF | 1PEG -Chloroalkane |
| I-314 | (Ac-KRVSF)-(2PEG-Chloroalkane) | Ac-KRVSF | 2PEG-Chloroalkane |
| I-315 | (Ac-KRVSF)-(3PEG-Chloroalkane) | Ac-KRVSF | 3PEG-Chloroalkane |
| I-316 | (Ac-KRVSF)-(4PEG-Chloroalkane) | Ac-KRVSF | 4PEG-Chloroalkane |
| I-317 | (Oct-KRVSF)-(0PEG-Chloroalkane) | Oct-KRVSF | 0PEG-Chloroalkane |
| I-318 | (Oct-KRVSF)-(1PEG-Chloroalkane) | Oct-KRVSF | 1PEG-Chloroalkane |
| I-319 | (Oct-KRVSF)-(2PEG-Chloroalkane) | Oct-KRVSF | 2PEG-Chloroalkane |
| I-320 | (Oct-KRVSF)-(3PEG-Chloroalkane) | Oct-KRVSF | 3PEG-Chloroalkane |
| I-321 | (Oct-KRVSF)-(4PEG-Chloroalkane) | Oct-KRVSF | 4PEG-Chloroalkane |
| I-322 | (Ac-KRVSF)-(0PEG-AKTallo) | Ac-KRVSF | 0PEG-AKTallo |
| I-323 | (Ac-KRVSF)-(1PEG-AKTallo) | Ac-KRVSF | 1PEG-AKTallo |
| I-324 | (Ac-KRVSF)-(2PEG-AKTallo) | Ac-KRVSF | 2PEG-AKTallo |
| I-325 | (Ac-KRVSF)-(3PEG-AKTallo) | Ac-KRVSF | 3PEG-AKTallo |
| I-326 | (Ac-KRVSF)-(4PEG-AKTallo) | Ac-KRVSF | 4PEG-AKTallo |
| I-327 | (Oct-KRVSF)-(0PEG-AKTallo) | Oct-KRVSF | 0PEG-AKTallo |
| I-328 | (Oct-KRVSF)-(1PEG-AKTallo) | Oct-KRVSF | 1PEG-AKTallo |
| I-329 | (Oct-KRVSF)-(2PEG-AKTallo) | Oct-KRVSF | 2PEG-AKTallo |
| I-330 | (Oct-KRVSF)-(3PEG-AKTallo) | Oct-KRVSF | 3PEG-AKTallo |
| I-331 | (Oct-KRVSF)-(4PEG-AKTallo) | Oct-KRVSF | 4PEG-AKTallo |
| I-332 | (Ac-KRVSF)-(0PEG-AKTcomp) | Ac-KRVSF | 0PEG-AKTcomp |
| I-333 | (Ac-KRVSF)-(1PEG-AKTcomp) | Ac-KRVSF | 1PEG-AKTcomp |
| I-334 | (Ac-KRVSF)-(2PEG-AKTcomp) | Ac-KRVSF | 2PEG-AKTcomp |
| I-335 | (Ac-KRVSF)-(3PEG-AKTcomp) | Ac-KRVSF | 3PEG-AKTcomp |
| I-336 | (Ac-KRVSF)-(4PEG-AKTcomp) | Ac-KRVSF | 4PEG-AKTcomp |
| I-337 | (Oct-KRVSF)-(0PEG-AKTcomp) | Oct-KRVSF | 0PEG-AKTcomp |
| I-338 | (Oct-KRVSF)-(1PEG-AKTcomp) | Oct-KRVSF | 1PEG-AKTcomp |
| I-339 | (Oct-KRVSF)-(2PEG-AKTcomp) | Oct-KRVSF | 2PEG-AKTcomp |
| I-340 | (Oct-KRVSF)-(3PEG-AKTcomp) | Oct-KRVSF | 3PEG-AKTcomp |
| I-341 | (Oct-KRVSF)-(4PEG-AKTcomp) | Oct-KRVSF | 4PEG-AKTcomp |
| I-342 | (Ac-KRVSF)-(0PEG-TBK1) | Ac-KRVSF | 0PEG-TBK1 |
| I-343 | (Ac-KRVSF)-(1PEG-TBK1) | Ac-KRVSF | 1PEG-TBK1 |
| I-344 | (Ac-KRVSF)-(2PEG-TBK1) | Ac-KRVSF | 2PEG-TBK1 |
| I-345 | (Ac-KRVSF)-(3PEG-TBK1) | Ac-KRVSF | 3PEG-TBK1 |
| I-346 | (Ac-KRVSF)-(4PEG-TBK1) | Ac-KRVSF | 4PEG-TBK1 |
| I-347 | (Oct-KRVSF)-(0PEG-TBK1) | Oct-KRVSF | 0PEG-TBK1 |
| I-348 | (Oct-KRVSF)-(1PEG-TBK1) | Oct-KRVSF | 1PEG-TBK1 |
| I-349 | (Oct-KRVSF)-(2PEG-TBK1) | Oct-KRVSF | 2PEG-TBK1 |
| I-350 | (Oct-KRVSF)-(3PEG-TBK1) | Oct-KRVSF | 3PEG-TBK1 |
| I-351 | (Oct-KRVSF)-(4PEG-TBK1) | Oct-KRVSF | 4PEG-TBK1 |
| I-352 | (Ac-KRVSF)-(0PEG-BRD4) | Ac-KRVSF | 0PEG-BRD4 |
| I-353 | (Ac-KRVSF)-(1PEG-BRD4) | Ac-KRVSF | 1PEG-BRD4 |
| I-354 | (Ac-KRVSF)-(2PEG-BRD4) | Ac-KRVSF | 2PEG-BRD4 |
| I-355 | (Ac-KRVSF)-(3PEG-BRD4) | Ac-KRVSF | 3PEG-BRD4 |
| I-356 | (Ac-KRVSF)-(4PEG-BRD4) | Ac-KRVSF | 4PEG-BRD4 |
| I-357 | (Oct-KRVSF)-(0PEG-BRD4) | Oct-KRVSF | 0PEG-BRD4 |
| I-358 | (Oct-KRVSF)-(1PEG-BRD4) | Oct-KRVSF | 1PEG-BRD4 |
| I-359 | (Oct-KRVSF)-(2PEG-BRD4) | Oct-KRVSF | 2PEG-BRD4 |
| I-360 | (Oct-KRVSF)-(3PEG-BRD4) | Oct-KRVSF | 3PEG-BRD4 |
| I-361 | (Oct-KRVSF)-(4PEG-BRD4) | Oct-KRVSF | 4PEG-BRD4 |

TABLE 16
Exemplary Product Amide Compounds

| | | Protein | |
|---|---|---|---|
| | | Phosphatase | |
| | | Ligand To Be | Linker-Target |
| | | Used In | Protein Ligand |
| | | Preparation | To Be Used In |
| | | of Product | Preparation of |
| Compound | | Amide | Product Amide |
| No. | Product Amide Compound | Compound | Compound |
| I-362 | (Ac-KSVTW)-(0PEG-Chloroalkane) | Ac-KSVTW | 0PEG-Chloroalkane |
| I-363 | (Ac-KSVTW)-(1PEG-Chloroalkane) | Ac-KSVTW | 1PEG-Chloroalkane |
| I-364 | (Ac-KSVTW)-(2PEG-Chloroalkane) | Ac-KSVTW | 2PEG-Chloroalkane |
| I-365 | (Ac-KSVTW)-(3PEG-Chloroalkane) | Ac-KSVTW | 3PEG-Chloroalkane |
| I-366 | (Ac-KSVTW)-(4PEG-Chloroalkane) | Ac-KSVTW | 4PEG-Chloroalkane |
| I-367 | (Oct-KSVTW)-(0PEG-Chloroalkane) | Oct-KSVTW | 0PEG-Chloroalkane |
| I-368 | (Oct-KSVTW)-(1PEG-Chloroalkane) | Oct-KSVTW | 1PEG-Chloroalkane |
| I-369 | (Oct-KSVTW)-(2PEG-Chloroalkane) | Oct-KSVTW | 2PEG-Chloroalkane |
| I-370 | (Oct-KSVTW)-(3PEG-Chloroalkane) | Oct-KSVTW | 3PEG-Chloroalkane |
| I-371 | (Oct-KSVTW)-(4PEG-Chloroalkane) | Oct-KSVTW | 4PEG-Chloroalkane |
| I-372 | (Ac-KSVTW)-(0PEG-AKTallo) | Ac-KSVTW | 0PEG-AKTallo |
| I-373 | (Ac-KSVTW)-(1PEG-AKTallo) | Ac-KSVTW | 1PEG-AKTallo |
| I-374 | (Ac-KSVTW)-(2PEG-AKTallo) | Ac-KSVTW | 2PEG-AKTallo |
| I-375 | (Ac-KSVTW)-(3PEG-AKTallo) | Ac-KSVTW | 3PEG-AKTallo |
| I-376 | (Ac-KSVTW)-(4PEG-AKTallo) | Ac-KSVTW | 4PEG-AKTallo |
| I-377 | (Oct-KSVTW)-(0PEG-AKTallo) | Oct-KSVTW | 0PEG-AKTallo |
| I-378 | (Oct-KSVTW)-(1PEG-AKTallo) | Oct-KSVTW | 1PEG-AKTallo |
| I-379 | (Oct-KSVTW)-(2PEG-AKTallo) | Oct-KSVTW | 2PEG-AKTallo |
| I-380 | (Oct-KSVTW)-(3PEG-AKTallo) | Oct-KSVTW | 3PEG-AKTallo |
| I-381 | (Oct-KSVTW)-(4PEG-AKTallo) | Oct-KSVTW | 4PEG-AKTallo |
| I-382 | (Ac-KSVTW)-(0PEG-AKTcomp) | Ac-KSVTW | 0PEG-AKTcomp |
| I-383 | (Ac-KSVTW)-(1PEG-AKTcomp) | Ac-KSVTW | 1PEG-AKTcomp |
| I-384 | (Ac-KSVTW)-(2PEG-AKTcomp) | Ac-KSVTW | 2PEG-AKTcomp |
| I-385 | (Ac-KSVTW)-(3PEG-AKTcomp) | Ac-KSVTW | 3PEG-AKTcomp |
| I-386 | (Ac-KSVTW)-(4PEG-AKTcomp) | Ac-KSVTW | 4PEG-AKTcomp |
| I-387 | (Oct-KSVTW)-(0PEG-AKTcomp) | Oct-KSVTW | 0PEG-AKTcomp |
| I-388 | (Oct-KSVTW)-(1PEG-AKTcomp) | Oct-KSVTW | 1PEG-AKTcomp |
| I-389 | (Oct-KSVTW)-(2PEG-AKTcomp) | Oct-KSVTW | 2PEG-AKTcomp |
| I-390 | (Oct-KSVTW)-(3PEG-AKTcomp) | Oct-KSVTW | 3PEG-AKTcomp |
| I-391 | (Oct-KSVTW)-(4PEG-AKTcomp) | Oct-KSVTW | 4PEG-AKTcomp |
| I-392 | (Ac-KSVTW)-(0PEG-TBK1) | Ac-KSVTW | 0PEG-TBK1 |
| I-393 | (Ac-KSVTW)-(1PEG-TBK1) | Ac-KSVTW | 1PEG-TBK1 |
| I-394 | (Ac-KSVTW)-(2PEG-TBK1) | Ac-KSVTW | 2PEG-TBK1 |
| I-395 | (Ac-KSVTW)-(3PEG-TBK1) | Ac-KSVTW | 3PEG-TBK1 |
| I-396 | (Ac-KSVTW)-(4PEG-TBK1) | Ac-KSVTW | 4PEG-TBK1 |
| I-397 | (Oct-KSVTW)-(0PEG-TBK1) | Oct-KSVTW | 0PEG-TBK1 |
| I-398 | (Oct-KSVTW)-(1PEG-TBK1) | Oct-KSVTW | 1PEG-TBK1 |
| I-399 | (Oct-KSVTW)-(2PEG-TBK1) | Oct-KSVTW | 2PEG-TBK1 |
| I-400 | (Oct-KSVTW)-(3PEG-TBK1) | Oct-KSVTW | 3PEG-TBK1 |
| I-401 | (Oct-KSVTW)-(4PEG-TBK1) | Oct-KSVTW | 4PEG-TBK1 |
| I-402 | (Ac-KSVTW)-(0PEG-BRD4) | Ac-KSVTW | 0PEG-BRD4 |
| I-403 | (Ac-KSVTW)-(1PEG-BRD4) | Ac-KSVTW | 1PEG-BRD4 |
| I-404 | (Ac-KSVTW)-(2PEG-BRD4) | Ac-KSVTW | 2PEG-BRD4 |
| I-405 | (Ac-KSVTW)-(3PEG-BRD4) | Ac-KSVTW | 3PEG-BRD4 |
| I-406 | (Ac-KSVTW)-(4PEG-BRD4) | Ac-KSVTW | 4PEG-BRD4 |
| I-407 | (0ct-KSVTW)-(0PEG-BRD4) | Oct-KSVTW | 0PEG-BRD4 |
| I-408 | (Oct-KSVTW)-(1PEG-BRD4) | Oct-KSVTW | 1PEG-BRD4 |
| I-409 | (0ct-KSVTW)-(2PEG-BRD4) | Oct-KSVTW | 2PEG-BRD4 |
| I-410 | (Oct-KSVTW)-(3PEG-BRD4) | Oct-KSVTW | 3PEG-BRD4 |
| I-411 | (Oct-KSVTW)-(4PEG-BRD4) | Oct-KSVTW | 4PEG-BRD4 |

TABLE 17
Chemical Structures for Abbreviations Used in the Description of Product Amide Compounds

| Abbreviation | Chemical Structure |
|---|---|
| (Ac-KRVSF)- | 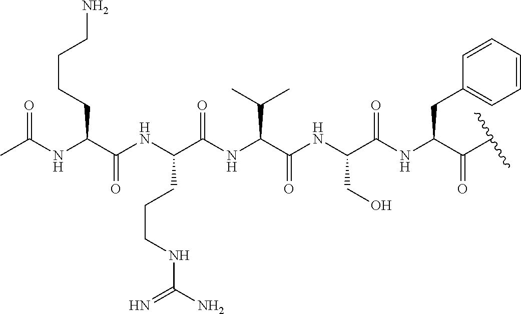 [SMILES: *C(=O)[C@H](Cc1ccccc1)NC(=O)[C@H](CO)NC(=O)[C@@H](NC(=O)[C@H](CCCNC(=N)N)NC(=O)[C@H](CCCCN)NC(C)=O)C(C)C] |
| (Oct-KRVSF)- | 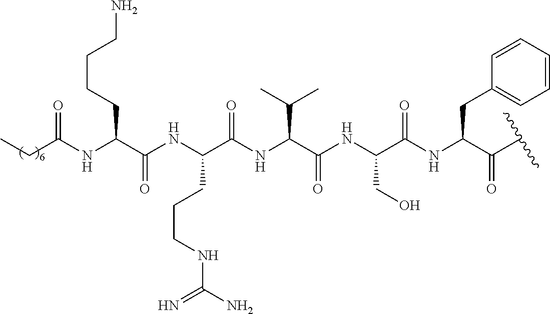 |
| (Ac-KSVTW)- | 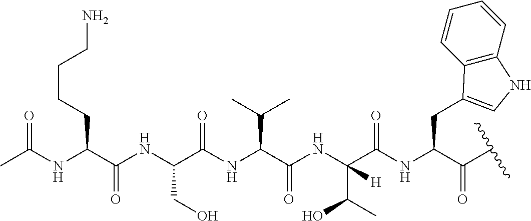 [SMILES: *C(=O)[C@H](Cc1c[nH]c2ccccc12)NC(=O)[C@@H](NC(=O)[C@@H](NC(=O)[C@H](CO)NC(=O)[C@H](CCCCN)NC(C)=O)C(C)C)[C@@H](C)O] |
| (Oct-KSVTW)- | 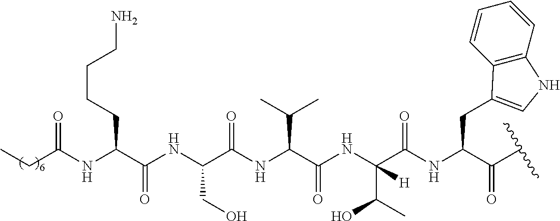 |

### Example 30: Preparation of Additional Product Amide Compounds from Carboxylic Acid-Containing Protein Phosphatase Ligands and Amine-Containing Linker-Target Protein Ligands

The product amide compounds in Table 14A and Table 16A below were prepared from carboxylic acid-containing protein phosphatase ligands and amine-containing linker-target protein ligands according to the general synthetic procedures described in Examples 2-11 and 22. Chemical structures for abbreviations used in the description of product amide compounds in Table 14A and Table 16A are provided in Tables 2, 8, 13, and 17.

TABLE 14A
Exemplary Product Amide Compounds

| | | Protein | Linker-Target | | | | |
|---|---|---|---|---|---|---|---|
| | | Phosphatase | Protein | | | | |
| | | Ligand | Ligand Used to | | | | |
| Com- | | Used to Prepare | Prepare Product | | LC/MS | | |
| pound | Product Amide | Product Amide | Amide | | MH + | | |
| No. | Compound | Compound | Compound | MW | (obs) | RT | Method |
| I-272 | (Ac-KRVHF)-(0PEG- | Ac-KRVHF | 0PEG-AKTallo | 1309.5 | 1310.4 | 1.79 | 10 |
| | AKTallo) | | | | | | |
| I-273 | (Ac-KRVHF)-(1PEG- | Ac-KRVHF | 1PEG-AKTallo | 1353.6 | 1353.5 | 1.81 | 10 |
| | AKTallo) | | | | | | |
| I-274 | (Ac-KRVHF)-(2PEG- | Ac-KRVHF | 2PEG-AKTallo | 1397.6 | 1397.5 | 1.83 | 10 |
| | AKTallo) | | | | | | |
| I-275 | (Ac-KRVHF)-(3PEG- | Ac-KRVHF | 3PEG-AKTallo | 1441.7 | 1441.8 | 1.85 | 10 |
| | AKTallo) | | | | | | |
| I-276 | (Ac-KRVHF)-(4PEG- | Ac-KRVHF | 4PEG-AKTallo | 1485.7 | 1485.6 | 1.87 | 10 |
| | AKTallo) | | | | | | |
| I-282 | (Ac-KRVHF)-(0PEG- | Ac-KRVHF | 0PEG-AKTcomp | 1282.0 | 1282.6 | 1.68 | 10 |
| | AKTcomp) | | | | | | |
| I-283 | (Ac-KRVHF)-(1PEG- | Ac-KRVHF | 1PEG-AKTcomp | 1326.0 | 1325.6 | 1.67 | 10 |
| | AKTcomp) | | | | | | |
| I-284 | (Ac-KRVHF)-(2PEG- | Ac-KRVHF | 2PEG-AKTcomp | 1370.1 | 685.3 | 1.69 | 10 |
| | AKTcomp) | | | | [M/2 + H] | | |
| I-285 | (Ac-KRVHF)-(3PEG- | Ac-KRVHF | 3PEG-AKTcomp | 1412.8 | 1414.6 | 1.71 | 10 |
| | AKTcomp) | | | | | | |
| I-286 | (Ac-KRVHF)-(4PEG- | Ac-KRVHF | 4PEG-AKTcomp | 1458.2 | 729.4 | 1.74 | 10 |
| | AKTcomp) | | | | [M/2 + H] | | |
| I-292 | (Ac-KRVHF)-(0PEG- | Ac-KRVHF | 0PEG-TBK1 | 1244.3 | 1243.5 | 2.11 | 01 |
| | TBK1) | | | | | | |
| I-293 | (Ac-KRVHF)-(1PEG- | Ac-KRVHF | 1PEG-TBK1 | 1288.3 | 1287.6 | 2.13 | 01 |
| | TBK1) | | | | | | |
| I-294 | (Ac-KRVHF)-(2PEG- | Ac-KRVHF | 2PEG-TBK1 | 1332.4 | 1333.4 | 2.15 | 01 |
| | TBK1) | | | | | | |
| I-295 | (Ac-KRVHF)-(3PEG- | Ac-KRVHF | 3PEG-TBK1 | 1332.4 | 1333.4 | 2.15 | 01 |
| | TBK1) | | | | | | |
| I-296 | (Ac-KRVHF)-(4PEG- | Ac-KRVHF | 4PEG-TBK1 | 1420.5 | 1421.7 | 2.62 | 00 |
| | TBK1) | | | | | | |

TABLE 16A
Exemplary Product Amide Compounds

| | | Protein | Linker-Target | | | | |
|---|---|---|---|---|---|---|---|
| | | Phosphatase | Protein | | | | |
| | | Ligand | Ligand Used to | | | | |
| Com- | | Used to Prepare | Prepare Product | | LC/MS | | |
| pound | Product Amide | Product Amide | Amide | | MH + | | |
| No. | Compound | Compound | Compound | MW | (obs) | RT | Method |
| 1-362 | (Ac-KSVTW)-(0PEG- | Ac-KSVTW | 0PEG-Chloroalkane | 924.52 | 924.4 | 2.11 | 10 |
| | Chloroalkane) | | | | | | |
| 1-363 | (Ac-KSVTW)-(1PEG- | Ac-KSVTW | 1PEG- | 968.58 | 968.4 | 2.10 | 10 |
| | Chloroalkane) | | Chloroalkane | | | | |
| 1-364 | (Ac-KSVTW)-(2PEG- | Ac-KSVTW | 2PEG-Chloroalkane | 1012.6 | 1012.3 | 2.15 | 10 |
| | Chloroalkane) | | | | | | |
| 1-365 | (Ac-KSVTW)-(3PEG- | Ac-KSVTW | 3PEG-Chloroalkane | 1056.7 | 1056.4 | 2.17 | 10 |
| | Chloroalkane) | | | | | | |
| 1-366 | (Ac-KSVTW)-(4PEG- | Ac-KSVTW | 4PEG-Chloroalkane | 1100.7 | 1100.5 | 2.50 | 01 |
| | Chloroalkane) | | | | | | |
| 1-367 | (Oct-KSVTW)-(0PEG- | Oct-KSVTW | 0PEG-Chloroalkane | 1007.6 | 1008.4 | 2.76 | 01 |
| | Chloroalkane) | | | | | | |
| 1-368 | (Oct-KSVTW)-(1PEG- | Oct-KSVTW | 1PEG- | 1052.7 | 1052.5 | 2.76 | 01 |
| | Chloroalkane) | | Chloroalkane | | | | |
| 1-369 | (Oct-KSVTW)-(2PEG- | Oct-KSVTW | 2PEG-Chloroalkane | 1096.8 | 1096.5 | 2.77 | 01 |
| | Chloroalkane) | | | | | | |
| I-370 | (Oct-KSVTW)-(3PEG- | Oct-KSVTW | 3PEG-Chloroalkane | 1140.8 | 1142.6 | 2.78 | 01 |
| | Chloroalkane) | | | | | | |
| 1-371 | (Oct-KSVTW)-(4PEG- | Oct-KSVTW | 4PEG-Chloroalkane | 1184.9 | 1184.6 | 2.79 | 01 |
| | Chloroalkane) | | | | | | |

### Example 31: Additional Product Amide Compounds for Preparation from Carboxylic Acid-Containing Target Protein Ligand-Succinates and Amine-Containing Linker-Protein Phosphatase Ligands

Product amide compounds in Tables 18-20 below may be prepared from carboxylic acid-containing target protein ligand-succinates (or target-protein ligands, in the case of compound BRD4) and amine-containing linker-protein phosphatase ligands according to the general synthetic procedures described in Examples 2-24. Chemical structures for abbreviations used in the description of product amide compounds in Tables 18-20 below are provided in Table 5 and Table 21.

TABLE 18
Exemplary Product Amide Compounds

| | | Target Protein | |
|---|---|---|---|
| | | Ligand-Succinate | Linker-Protein |
| | | or Target Protein | Phosphatase Ligand |
| | | Ligand For Use In | For Use In |
| Compound | | Preparing Product | Preparing Product |
| No. | Product Amide Compound | Amide Compound | Amide Compound |
| I-412 | (AKTallo-succinate)-(0PEG-KRVSF-NMe) | AKTallo-succinate | 0PEG-KRVSF-NMe |
| I-413 | (AKTallo-succinate)-(0PEG-KRVSF-NOct) | AKTallo-succinate | 0PEG-KRVSF-NOct |
| I-414 | (AKTallo-succinate)-(1PEG-KRVSF-NMe) | AKTallo-succinate | 1PEG-KRVSF-NMe |
| I-415 | (AKTallo-succinate)-(1PEG-KRVSF-NOct) | AKTallo-succinate | 1PEG-KRVSF-NOct |
| I-416 | (AKTallo-succinate)-(2PEG-KRVSF-NMe) | AKTallo-succinate | 2PEG-KRVSF-NMe |
| I-417 | (AKTallo-succinate)-(2PEG-KRVSF-NOct) | AKTallo-succinate | 2PEG-KRVSF-NOct |
| I-418 | (AKTallo-succinate)-(3PEG-KRVSF-NMe) | AKTallo-succinate | 3PEG-KRVSF-NMe |
| I-419 | (AKTallo-succinate)-(3PEG-KRVSF-NOct) | AKTallo-succinate | 3PEG-KRVSF-NOct |
| I-420 | (AKTallo-succinate)-(4PEG-KRVSF-NMe) | AKTallo-succinate | 4PEG-KRVSF-NMe |
| I-421 | (AKTallo-succinate)-(4PEG-KRVSF-NOct) | AKTallo-succinate | 4PEG-KRVSF-NOct |
| I-422 | (AKTcomp-succinate)-(0PEG-KRVSF-NMe) | AKTcomp-succinate | 0PEG-KRVSF-NMe |
| I-423 | (AKTcomp-succinate)-(0PEG-KRVSF-NOct) | AKTcomp-succinate | 0PEG-KRVSF-NOct |
| I-424 | (AKTcomp-succinate)-(1PEG-KRVSF-NMe) | AKTcomp-succinate | 1PEG-KRVSF-NMe |
| I-425 | (AKTcomp-succinate)-(1PEG-KRVSF-NOct) | AKTcomp-succinate | 1PEG-KRVSF-NOct |
| I-426 | (AKTcomp-succinate)-(2PEG-KRVSF-NMe) | AKTcomp-succinate | 2PEG-KRVSF-NMe |
| I-427 | (AKTcomp-succinate)-(2PEG-KRVSF-NOct) | AKTcomp-succinate | 2PEG-KRVSF-NOct |
| I-428 | (AKTcomp-succinate)-(3PEG-KRVSF-NMe) | AKTcomp-succinate | 3PEG-KRVSF-NMe |
| I-429 | (AKTcomp-succinate)-(3PEG-KRVSF-NOct) | AKTcomp-succinate | 3PEG-KRVSF-NOct |
| I-430 | (AKTcomp-succinate)-(4PEG-KRVSF-NMe) | AKTcomp-succinate | 4PEG-KRVSF-NMe |
| I-431 | (AKTcomp-succinate)-(4PEG-KRVSF-NOct) | AKTcomp-succinate | 4PEG-KRVSF-NOct |
| I-432 | (Chloroalkane-succinate)-(0PEG-KRVSF-NMe) | Chloroalkane-succinate | 0PEG-KRVSF-NMe |
| I-433 | (Chloroalkane-succinate)-(0PEG-KRVSF-NOct) | Chloroalkane-succinate | 0PEG-KRVSF-NOct |
| I-434 | (Chloroalkane-succinate)-(1PEG-KRVSF-NMe) | Chloroalkane-succinate | 1PEG-KRVSF-NMe |
| I-435 | (Chloroalkane-succinate)-(1PEG-KRVSF-NOct) | Chloroalkane-succinate | 1PEG-KRVSF-NOct |
| I-436 | (Chloroalkane-succinate)-(2PEG-KRVSF-NMe) | Chloroalkane-succinate | 2PEG-KRVSF-NMe |
| I-437 | (Chloroalkane-succinate)-(2PEG-KRVSF-NOct) | Chloroalkane-succinate | 2PEG-KRVSF-NOct |
| I-438 | (Chloroalkane-succinate)-(3PEG-KRVSF-NMe) | Chloroalkane-succinate | 3PEG-KRVSF-NMe |
| I-439 | (Chloroalkane-succinate)-(3PEG-KRVSF-NOct) | Chloroalkane-succinate | 3PEG-KRVSF-NOct |
| I-440 | (Chloroalkane-succinate)-(4PEG-KRVSF-NMe) | Chloroalkane-succinate | 4PEG-KRVSF-NMe |
| I-441 | (Chloroalkane-succinate)-(4PEG-KRVSF-NOct) | Chloroalkane-succinate | 4PEG-KRVSF-NOct |
| I-442 | (TBK1-succinate)-(0PEG-KRVSF-NMe) | TBK1-succinate | 0PEG-KRVSF-NMe |
| I-443 | (TBK1-succinate)-(0PEG-KRVSF-NOct) | TBK1-succinate | 0PEG-KRVSF-NOct |
| I-444 | (TBK1-succinate)-(1PEG-KRVSF-NMe) | TBK1-succinate | 1PEG-KRVSF-NMe |
| I-445 | (TBK1-succinate)-(1PEG-KRVSF-NOct) | TBK1-succinate | 1PEG-KRVSF-NOct |
| I-446 | (TBK1-succinate)-(2PEG-KRVSF-NMe) | TBK1-succinate | 2PEG-KRVSF-NMe |
| I-447 | (TBK1-succinate)-(2PEG-KRVSF-NOct) | TBK1-succinate | 2PEG-KRVSF-NOct |
| I-448 | (TBK1-succinate)-(3PEG-KRVSF-NMe) | TBK1-succinate | 3PEG-KRVSF-NMe |
| I-449 | (TBK1-succinate)-(3PEG-KRVSF-NOct) | TBK1-succinate | 3PEG-KRVSF-NOct |
| I-450 | (TBK1-succinate)-(4PEG-KRVSF-NMe) | TBK1-succinate | 4PEG-KRVSF-NMe |
| I-451 | (TBK1-succinate)-(4PEG-KRVSF-NOct) | TBK1-succinate | 4PEG-KRVSF-NOct |
| I-452 | (BRD4)-(0PEG-KRVSF-NMe) | BRD4 | 0PEG-KRVSF-NMe |
| I-453 | (BRD4)-(0PEG-KRVSF-NOct) | BRD4 | 0PEG-KRVSF-NOct |
| I-454 | (BRD4)-(1PEG-KRVSF-NMe) | BRD4 | 1PEG-KRVSF-NMe |
| I-455 | (BRD4)-(1PEG-KRVSF-NOct) | BRD4 | 1PEG-KRVSF-NOct |
| I-456 | (BRD4)-(2PEG-KRVSF-NMe) | BRD4 | 2PEG-KRVSF-NMe |
| I-457 | (BRD4)-(2PEG-KRVSF-NOct) | BRD4 | 2PEG-KRVSF-NOct |
| I-458 | (BRD4)-(3PEG-KRVSF-NMe) | BRD4 | 3PEG-KRVSF-NMe |
| I-459 | (BRD4)-(3PEG-KRVSF-NOct) | BRD4 | 3PEG-KRVSF-NOct |
| I-460 | (BRD4)-(4PEG-KRVSF-NMe) | BRD4 | 4PEG-KRVSF-NMe |
| I-461 | (BRD4)-(4PEG-KRVSF-NOct) | BRD4 | 4PEG-KRVSF-NOct |

TABLE 19
Exemplary Product Amide Compounds

| | | Target Protein | |
|---|---|---|---|
| | | Ligand-Succinate or | Linker-Protein |
| | | Target Protein Ligand | Phosphatase Ligand |
| | | Ligand For Use In | Ligand For Use In |
| Compound | | Preparing Product | Preparing Product |
| No. | Product Amide Compound | Amide Compound | Amide Compound |
| I-462 | (AKTallo-succinate)-(0PEG-KRVHF-NMe) | AKTallo-succinate | 0PEG-KRVHF-NMe |
| I-463 | (AKTallo-succinate)-(0PEG-KRVHF-NOct) | AKTallo-succinate | 0PEG-KRVHF-NOct |
| I-464 | (AKTallo-succinate)-(1PEG-KRVHF-NMe) | AKTallo-succinate | 1PEG-KRVHF-NMe |
| I-465 | (AKTallo-succinate)-(1PEG-KRVHF-NOct) | AKTallo-succinate | 1PEG-KRVHF-NOct |
| I-466 | (AKTallo-succinate)-(2PEG-KRVHF-NMe) | AKTallo-succinate | 2PEG-KRVHF-NMe |
| I-467 | (AKTallo-succinate)-(2PEG-KRVHF-NOct) | AKTallo-succinate | 2PEG-KRVHF-NOct |
| I-468 | (AKTallo-succinate)-(3PEG-KRVHF-NMe) | AKTallo-succinate | 3PEG-KRVHF-NMe |
| I-469 | (AKTallo-succinate)-(3PEG-KRVHF-NOct) | AKTallo-succinate | 3PEG-KRVHF-NOct |
| I-470 | (AKTallo-succinate)-(4PEG-KRVHF-NMe) | AKTallo-succinate | 4PEG-KRVHF-NMe |
| I-471 | (AKTallo-succinate)-(4PEG-KRVHF-NOct) | AKTallo-succinate | 4PEG-KRVHF-NOct |
| I-472 | (AKTcomp-succinate)-(0PEG-KRVHF-NMe) | AKTcomp-succinate | 0PEG-KRVHF-NMe |
| I-473 | (AKTcomp-succinate)-(0PEG-KRVHF-NOct) | AKTcomp-succinate | 0PEG-KRVHF-NOct |
| I-474 | (AKTcomp-succinate)-(1PEG-KRVHF-NMe) | AKTcomp-succinate | 1PEG-KRVHF-NMe |
| I-475 | (AKTcomp-succinate)-(1PEG-KRVHF-NOct) | AKTcomp-succinate | 1PEG-KRVHF-NOct |
| I-476 | (AKTcomp-succinate)-(2PEG-KRVHF-NMe) | AKTcomp-succinate | 2PEG-KRVHF-NMe |
| I-477 | (AKTcomp-succinate)-(2PEG-KRVHF-NOct) | AKTcomp-succinate | 2PEG-KRVHF-NOct |
| I-478 | (AKTcomp-succinate)-(3PEG-KRVHF-NMe) | AKTcomp-succinate | 3PEG-KRVHF-NMe |
| I-479 | (AKTcomp-succinate)-(3PEG-KRVHF-NOct) | AKTcomp-succinate | 3PEG-KRVHF-NOct |
| I-480 | (AKTcomp-succinate)-(4PEG-KRVHF-NMe) | AKTcomp-succinate | 4PEG-KRVHF-NM |
| I-481 | (AKTcomp-succinate)-(4PEG-KRVHF-NOct) | AKTcomp-succinate | 4PEG-KRVHF-NOct |
| I-482 | (Chloroalkane-succinate)-(0PEG-KRVHF-NMe) | Chloroalkane-succinate | 0PEG-KRVHF-NMe |
| I-483 | (Chloroalkane-succinate)-(0PEG-KRVHF-NOct) | Chloroalkane-succinate | 0PEG-KRVHF-NOct |
| I-484 | (Chloroalkane-succinate)-(1PEG-KRVHF-NMe) | Chloroalkane-succinate | 1PEG-KRVHF-NMe |
| I-485 | (Chloroalkane-succinate)-(1PEG-KRVHF-NOct) | Chloroalkane-succinate | 1PEG-KRVHF-NOct |
| I-486 | (Chloroalkane-succinate)-(2PEG-KRVHF-NMe) | Chloroalkane-succinate | 2PEG-KRVHF-NMe |
| I-487 | (Chloroalkane-succinate)-(2PEG-KRVHF-NOct) | Chloroalkane-succinate | 2PEG-KRVHF-NOct |
| I-488 | (Chloroalkane-succinate)-(3PEG-KRVHF-NMe) | Chloroalkane-succinate | 3PEG-KRVHF-NMe |
| I-489 | (Chloroalkane-succinate)-(3PEG-KRVHF-NOct) | Chloroalkane-succinate | 3PEG-KRVHF-NOct |
| I-490 | (Chloroalkane-succinate)-(4PEG-KRVHF-NMe) | Chloroalkane-succinate | 4PEG-KRVHF-NMe |
| I-491 | (Chloroalkane-succinate)-(4PEG-KRVHF-NOct) | Chloroalkane-succinate | 4PEG-KRVHF-NOct |
| I-492 | (TBK1-succinate)-(0PEG-KRVHF-NMe) | TBK1-succinate | 0PEG-KRVHF-NMe |
| I-493 | (TBK1-succinate)-(0PEG-KRVHF-NOct) | TBK1-succinate | 0PEG-KRVHF-NOct |
| I-494 | (TBK1-succinate)-(1PEG-KRVHF-NMe) | TBK1-succinate | 1PEG-KRVHF-NMe |
| I-495 | (TBK1-succinate)-(1PEG-KRVHF-NOct) | TBK1-succinate | 1PEG-KRVHF-NOct |
| I-496 | (TBK1-succinate)-(2PEG-KRVHF-NMe) | TBK1-succinate | 2PEG-KRVHF-NMe |
| I-497 | (TBK1-succinate)-(2PEG-KRVHF-NOct) | TBK1-succinate | 2PEG-KRVHF-NOct |
| I-498 | (TBK1-succinate)-(3PEG-KRVHF-NMe) | TBK1-succinate | 3PEG-KRVHF-NMe |
| I-499 | (TBK1-succinate)-(3PEG-KRVHF-NOct) | TBK1-succinate | 3PEG-KRVHF-NOct |
| I-500 | (TBK1-succinate)-(4PEG-KRVHF-NMe) | TBK1-succinate | 4PEG-KRVHF-NMe |
| I-501 | (TBK1-succinate)-(4PEG-KRVHF-NOct) | TBK1-succinate | 4PEG-KRVHF-NOct |
| I-502 | (BRD4)-(0PEG-KRVHF-NMe) | BRD4 | 0PEG-KRVHF-NMe |
| I-503 | (BRD4)-(0PEG-KRVHF-NOct) | BRD4 | 0PEG-KRVHF-NOct |
| I-504 | (BRD4)-(1PEG-KRVHF-NMe) | BRD4 | 1PEG-KRVHF-NMe |
| I-505 | (BRD4)-(1PEG-KRVHF-NOct) | BRD4 | 1PEG-KRVHF-NOct |
| I-506 | (BRD4)-(2PEG-KRVHF-NMe) | BRD4 | 2PEG-KRVHF-NMe |
| I-507 | (BRD4)-(2PEG-KRVHF-NOct) | BRD4 | 2PEG-KRVHF-NOct |
| I-508 | (BRD4)-(3PEG-KRVHF-NMe) | BRD4 | 3PEG-KRVHF-NMe |
| I-509 | (BRD4)-(3PEG-KRVHF-NOct) | BRD4 | 3PEG-KRVHF-NOct |
| I-510 | (BRD4)-(4PEG-KRVHF-NMe) | BRD4 | 4PEG-KRVHF-NMe |
| I-511 | (BRD4)-(4PEG-KRVHF-NOct) | BRD4 | 4PEG-KRVHF-NOct |

TABLE 20
Exemplary Product Amide Compounds

| | | Target Protein | |
|---|---|---|---|
| | | Ligand-Succinate or | Linker-Protein |
| | | Target Protein Ligand | Phosphatase Ligand |
| | | Ligand For Use In | Ligand For Use In |
| Compound | | Preparing Product | Preparing Product |
| No. | Product Amide Compound | Amide Compound | Amide Compound |
| I-512 | (AKTallo-succinate)-(0PEG-KSVTW-NMe) | AKTallo-succinate | 0PEG-KSVTW-NMe |
| I-513 | (AKTallo-succinate)-(0PEG-KSVTW-NOct) | AKTallo-succinate | 0PEG-KSVTW-NOct |
| I-514 | (AKTallo-succinate)-(1PEG-KSVTW-NMe) | AKTallo-succinate | 1PEG-KSVTW-NMe |
| I-515 | (AKTallo-succinate)-(1PEG-KSVTW-NOct) | AKTallo-succinate | 1PEG-KSVTW-NOct |
| I-516 | (AKTallo-succinate)-(2PEG-KSVTW-NMe) | AKTallo-succinate | 2PEG-KSVTW-NMe |
| I-517 | (AKTallo-succinate)-(2PEG-KSVTW-NOct) | AKTallo-succinate | 2PEG-KSVTW-NOct |
| I-518 | (AKTallo-succinate)-(3PEG-KSVTW-NMe) | AKTallo-succinate | 3PEG-KSVTW-NMe |
| I-519 | (AKTallo-succinate)-(3PEG-KSVTW-NOct) | AKTallo-succinate | 3PEG-KSVTW-NOct |
| I-520 | (AKTallo-succinate)-(4PEG-KSVTW-NMe) | AKTallo-succinate | 4PEG-KSVTW-NMe |
| I-521 | (AKTallo-succinate)-(4PEG-KSVTW-NOct) | AKTallo-succinate | 4PEG-KSVTW-NOct |
| I-522 | (AKTcomp-succinate)-(0PEG-KSVTW-NMe) | AKTcomp-succinate | 0PEG-KSVTW-NMe |
| I-523 | (AKTcomp-succinate)-(0PEG-KSVTW-NOct) | AKTcomp-succinate | 0PEG-KSVTW-NOct |
| I-524 | (AKTcomp-succinate)-(1PEG-KSVTW-NMe) | AKTcomp-succinate | 1PEG-KSVTW-NMe |
| I-525 | (AKTcomp-succinate)-(1PEG-KSVTW-NOct) | AKTcomp-succinate | 1PEG-KSVTW-NOct |
| I-526 | (AKTcomp-succinate)-(2PEG-KSVTW-NMe) | AKTcomp-succinate | 2PEG-KSVTW-NMe |
| I-527 | (AKTcomp-succinate)-(2PEG-KSVTW-NOct) | AKTcomp-succinate | 2PEG-KSVTW-NOct |
| I-528 | (AKTcomp-succinate)-(3PEG-KSVTW-NMe) | AKTcomp-succinate | 3PEG-KSVTW-NMe |
| I-529 | (AKTcomp-succinate)-(3PEG-KSVTW-NOct) | AKTcomp-succinate | 3PEG-KSVTW-NOct |
| I-530 | (AKTcomp-succinate)-(4PEG-KSVTW-NMe) | AKTcomp-succinate | 4PEG-KSVTW-NMe |
| I-531 | (AKTcomp-succinate)-(4PEG-KSVTW-NOct) | AKTcomp-succinate | 4PEG-KSVTW-NOct |
| I-532 | (Chloroalkane-succinate)-(0PEG-KSVTW-NMe) | Chloroalkane-succinate | 0PEG-KSVTW-NMe |
| I-533 | (Chloroalkane-succinate)-(0PEG-KSVTW-NOct) | Chloroalkane-succinate | 0PEG-KSVTW-NOct |
| I-534 | (Chloroalkane-succinate)-(1PEG-KSVTW-NMe) | Chloroalkane-succinate | 1PEG-KSVTW-NMe |
| I-535 | (Chloroalkane-succinate)-(1PEG-KSVTW-NOct) | Chloroalkane-succinate | 1PEG-KSVTW-NOct |
| I-536 | (Chloroalkane-succinate)-(2PEG-KSVTW-NMe) | Chloroalkane-succinate | 2PEG-KSVTW-NMe |
| I-537 | (Chloroalkane-succinate)-(2PEG-KSVTW-NOct) | Chloroalkane-succinate | 2PEG-KSVTW-NOct |
| I-538 | (Chloroalkane-succinate)-(3PEG-KSVTW-NMe) | Chloroalkane-succinate | 3PEG-KSVTW-NMe |
| I-539 | (Chloroalkane-succinate)-(3PEG-KSVTW-NOct) | Chloroalkane-succinate | 3PEG-KSVTW-NOct |
| I-540 | (Chloroalkane-succinate)-(4PEG-KSVTW-NMe) | Chloroalkane-succinate | 4PEG-KSVTW-NMe |
| I-541 | (Chloroalkane-succinate)-(4PEG-KSVTW-NOct) | Chloroalkane-succinate | 4PEG-KSVTW-NOct |
| I-542 | (TBK1-succinate)-(0PEG-KSVTW-NMe) | TBK1-succinate | 0PEG-KSVTW-NMe |
| I-543 | (TBK1-succinate)-(0PEG-KSVTW-NOct) | TBK1-succinate | 0PEG-KSVTW-NOct |
| I-544 | (TBK1-succinate)-(1PEG-KSVTW-NMe) | TBK1-succinate | 1PEG-KSVTW-NMe |
| I-545 | (TBK1-succinate)-(1PEG-KSVTW-NOct) | TBK1-succinate | 1PEG-KSVTW-NOct |
| I-546 | (TBK1-succinate)-(2PEG-KSVTW-NMe) | TBK1-succinate | 2PEG-KSVTW-NMe |
| I-547 | (TBK1-succinate)-(2PEG-KSVTW-NOct) | TBK1-succinate | 2PEG-KSVTW-NOct |
| I-548 | (TBK1-succinate)-(3PEG-KSVTW-NMe) | TBK1-succinate | 3PEG-KSVTW-NMe |
| I-549 | (TBK1-succinate)-(3PEG-KSVTW-NOct) | TBK1-succinate | 3PEG-KSVTW-NOct |
| I-550 | (TBK1-succinate)-(4PEG-KSVTW-NMe) | TBK1-succinate | 4PEG-KSVTW-NMe |
| I-551 | (TBK1-succinate)-(4PEG-KSVTW-NOct) | TBK1-succinate | 4PEG-KSVTW-NOct |
| I-552 | (BRD4)-(0PEG-KSVTW-NMe) | BRD4 | 0PEG-KSVTW-NMe |
| I-553 | (BRD4)-(0PEG-KSVTW-NOct) | BRD4 | 0PEG-KSVTW-NOct |
| I-554 | (BRD4)-(1PEG-KSVTW-NMe) | BRD4 | 1PEG-KSVTW-NMe |
| I-555 | (BRD4)-(1PEG-KSVTW-NOct) | BRD4 | 1PEG-KSVTW-NOct |
| I-556 | (BRD4)-(2PEG-KSVTW-NMe) | BRD4 | 2PEG-KSVTW-NMe |
| I-557 | (BRD4)-(2PEG-KSVTW-NOct) | BRD4 | 2PEG-KSVTW-NOct |
| I-558 | (BRD4)-(3PEG-KSVTW-NMe) | BRD4 | 3PEG-KSVTW-NMe |
| I-559 | (BRD4)-(3PEG-KSVTW-NOct) | BRD4 | 3PEG-KSVTW-NOct |
| I-560 | (BRD4)-(4PEG-KSVTW-NMe) | BRD4 | 4PEG-KSVTW-NMe |
| I-561 | (BRD4)-(4PEG-KSVTW-NOct) | BRD4 | 4PEG-KSVTW-NOct |

TABLE 21
Chemical Structures for Abbreviations Used in the Description of Product Amide Compounds

| Abbreviation | Chemical Structure |
|---|---|
| (BRD4)- | 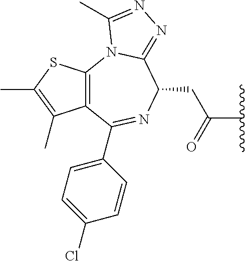 [SMILES: *C(=O)C[C@@H]1N=C(c2ccc(Cl)cc2)c2c(sc(C)c2C)-n2c(C)nnc21] |
| -(0PEG-KRVSF-NMe) | 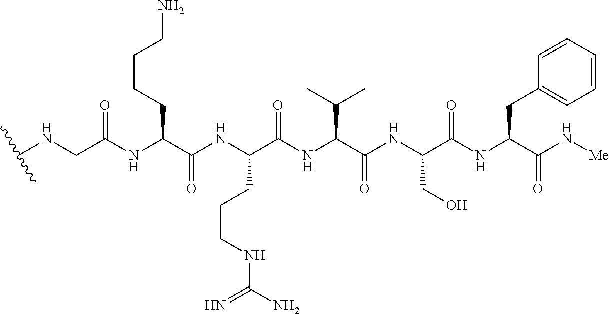 [SMILES: *NCC(=O)N[C@@H](CCCCN)C(=O)N[C@@H](CCCNC(=N)N)C(=O)N[C@H](C(=O)N[C@@H](CO)C(=O)N[C@@H](Cc1ccccc1)C(=O)NC)C(C)C] |
| -(1PEG-KRVSF-NMe) | 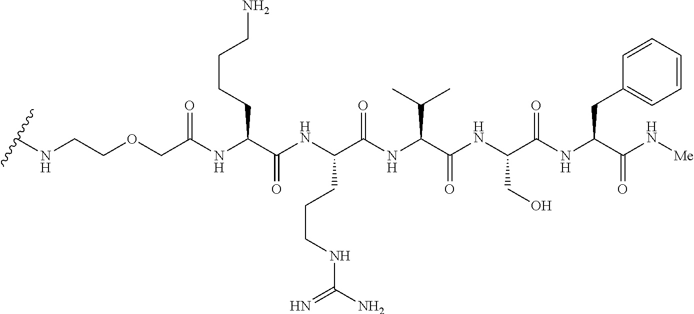 [SMILES: *NCCOCC(=O)N[C@@H](CCCCN)C(=O)N[C@@H](CCCNC(=N)N)C(=O)N[C@H](C(=O)N[C@@H](CO)C(=O)N[C@@H](Cc1ccccc1)C(=O)NC)C(C)C] |
| -(2PEG-KRVSF-NMe) | 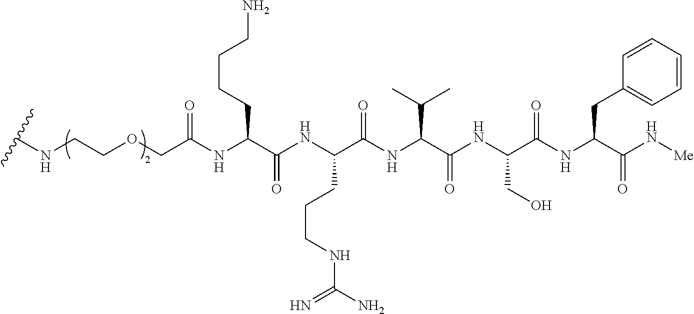 |
| -(3PEG-KRVSF-NMe) | 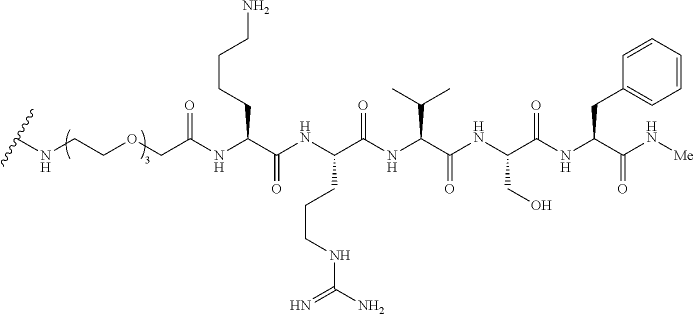 |
| -(4PEG-KRVSF-NMe) | 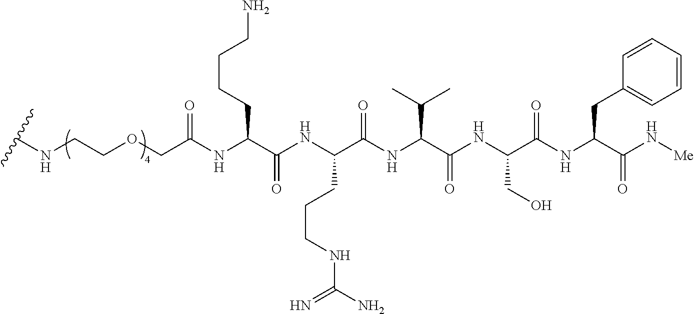 |
| -(0PEG-KRVSF-NOct) | 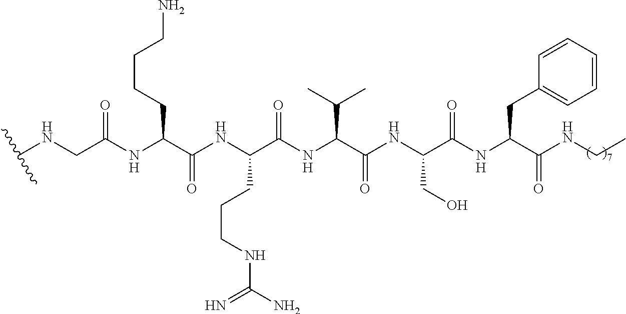 |
| -(1PEG-KRVSF-NOct) | 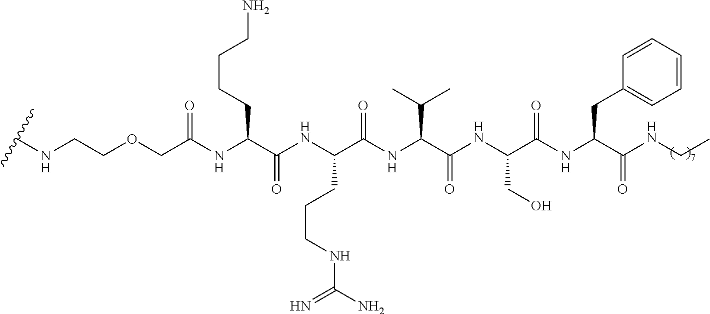 |
| -(2PEG-KRVSF-NOct) | 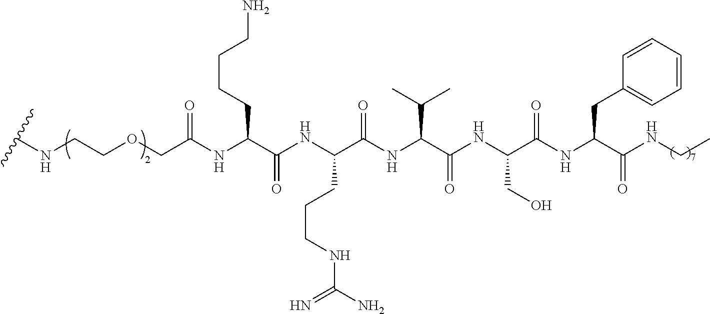 |
| -(3PEG-KRVSF-NOct) | 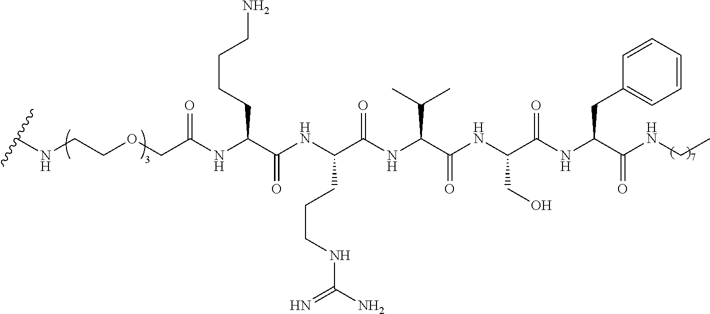 |
| -(4PEG-KRVSF-NOct) | 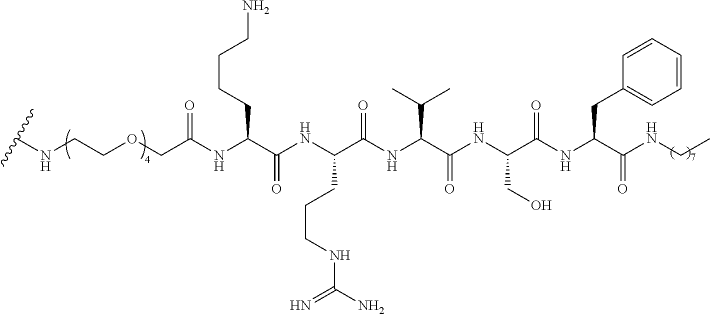 |
| -(0PEG-KRVHF-NMe) | 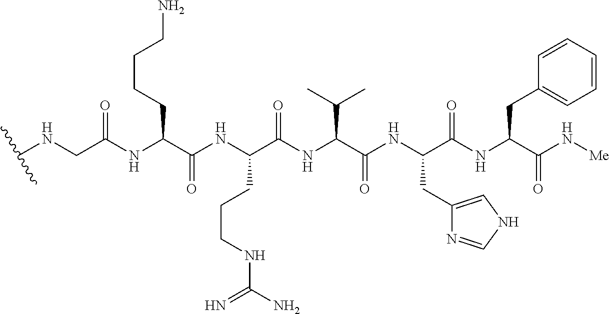 [SMILES: *NCC(=O)N[C@@H](CCCCN)C(=O)N[C@@H](CCCNC(=N)N)C(=O)N[C@H](C(=O)N[C@@H](Cc1c[nH]cn1)C(=O)N[C@@H](Cc1ccccc1)C(=O)NC)C(C)C] |
| -(1PEG-KRVHF-NMe) | 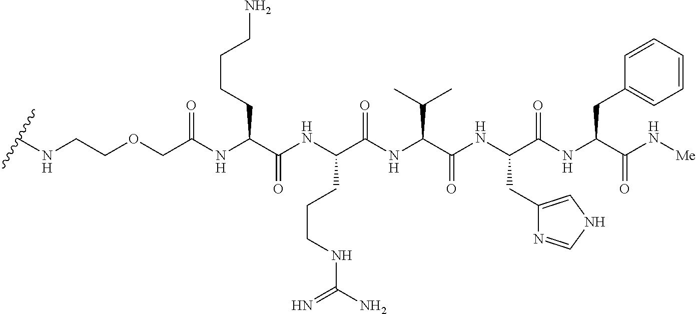 [SMILES: *NCCOCC(=O)N[C@@H](CCCCN)C(=O)N[C@@H](CCCNC(=N)N)C(=O)N[C@H](C(=O)N[C@@H](Cc1c[nH]cn1)C(=O)N[C@@H](Cc1ccccc1)C(=O)NC)C(C)C] |
| -(2PEG-KRVHF-NMe) | 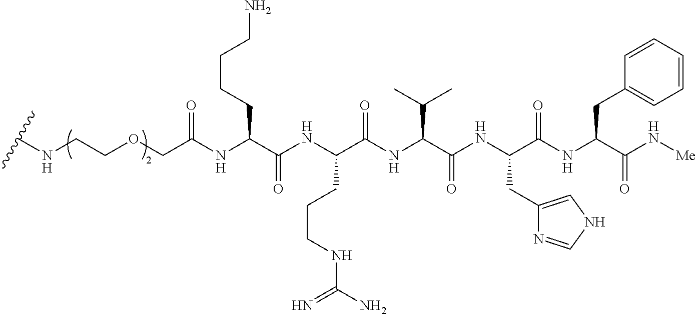 |
| -(3PEG-KRVHF-NMe) | 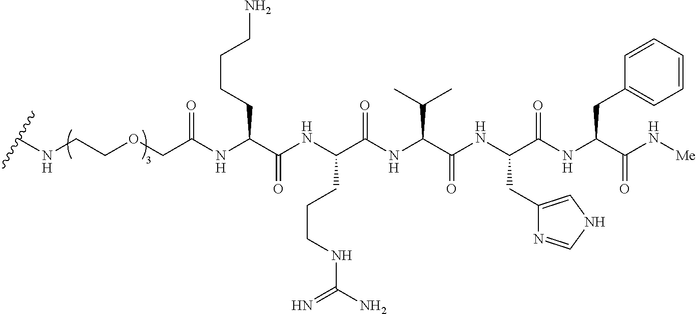 |
| -(4PEG-KRVHF-NMe) | 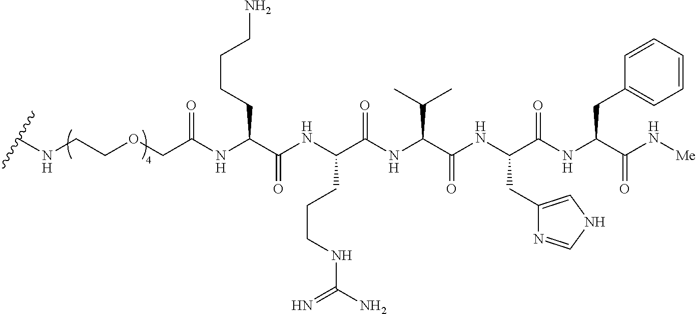 |
| -(0PEG-KRVHF-NOct) | 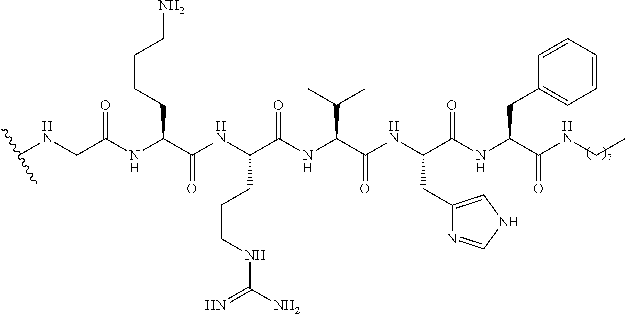 |
| -(1PEG-KRVHF-NOct) | 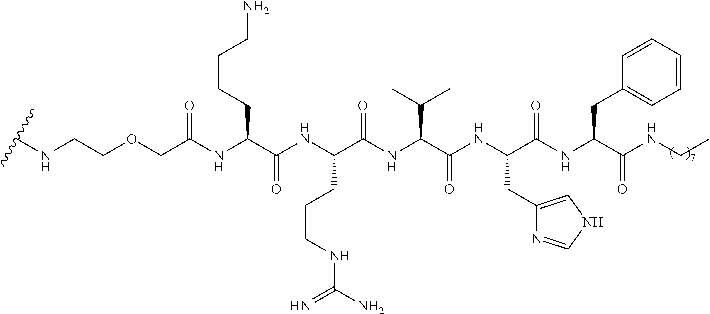 |
| -(2PEG-KRVHF-NOct) | 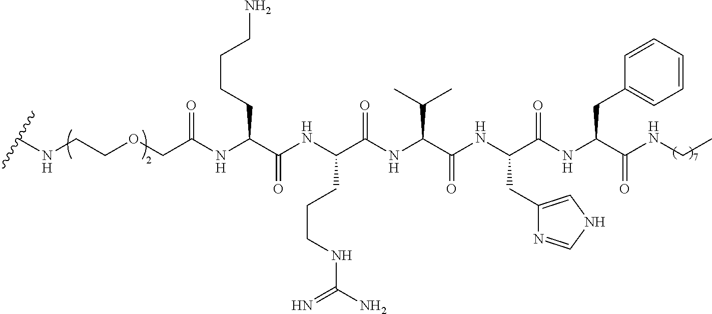 |
| -(3PEG-KRVHF-NOct) | 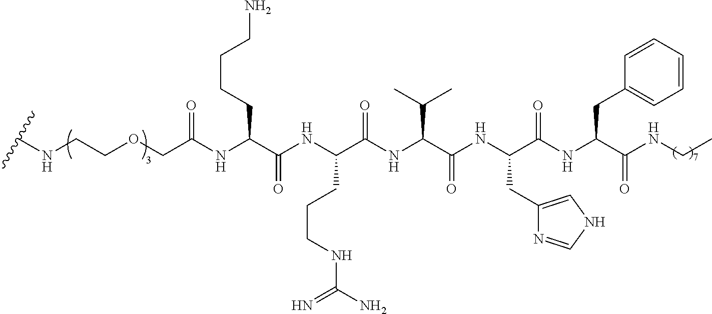 |
| -(4PEG-KRVHF-NOct) | 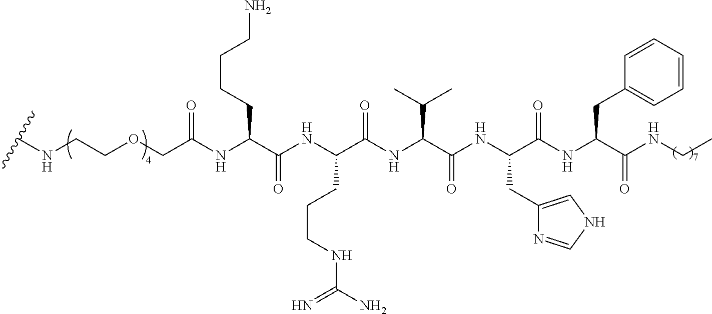 |
| -(0PEG-KSVTW-NMe) | 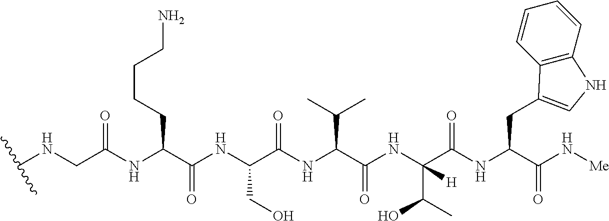 [SMILES: *NCC(=O)N[C@@H](CCCCN)C(=O)N[C@@H](CO)C(=O)N[C@H](C(=O)N[C@H](C(=O)N[C@@H](Cc1c[nH]c2ccccc12)C(=O)NC)[C@@H](C)O)C(C)C] |
| -(1PEG-KSVTW-NMe) | 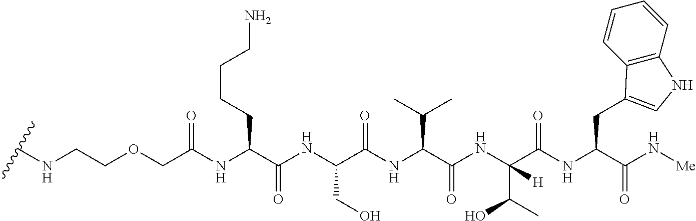 [SMILES: *NCCOCC(=O)N[C@@H](CCCCN)C(=O)N[C@@H](CO)C(=O)N[C@H](C(=O)N[C@H](C(=O)N[C@@H](Cc1c[nH]c2ccccc12)C(=O)NC)[C@@H](C)O)C(C)C] |
| -(2PEG-KSVTW-NMe) | 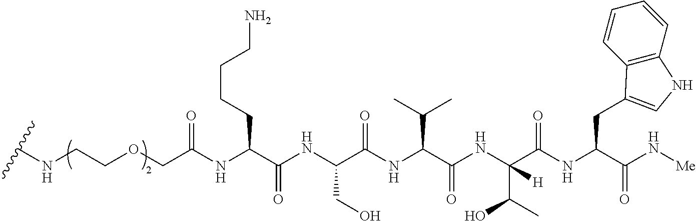 |
| -(3PEG-KSVTW-NMe) | 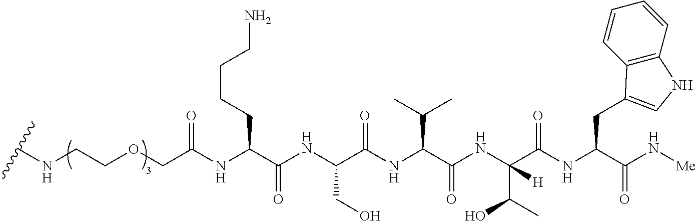 |
| -(4PEG-KSVTW-NMe) | 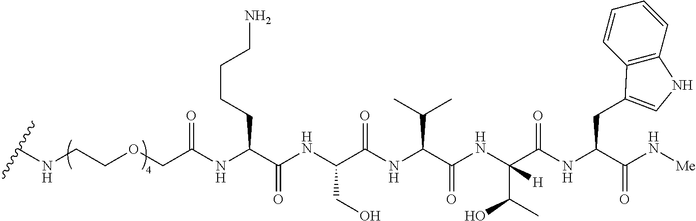 |
| -(0PEG-KSVTW-NOct) | 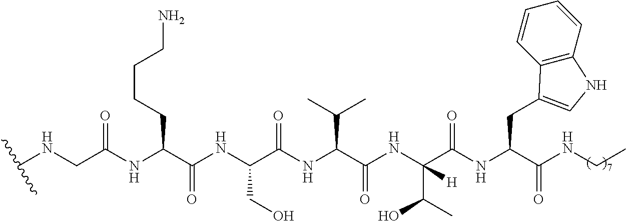 |
| -(1PEG-KSVTW-NOct) | 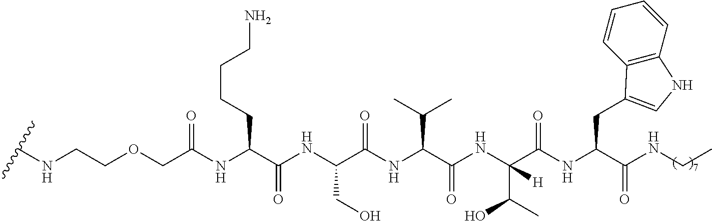 |
| -(2PEG-KSVTW-NOct) | 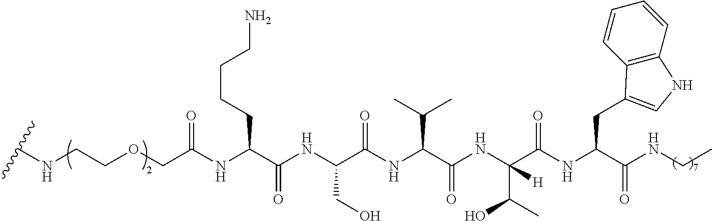 |
| -(3PEG-KSVTW-NOct) | 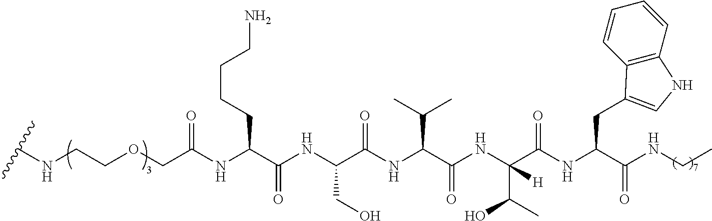 |
| -(4PEG-KSVTW-NOct) | 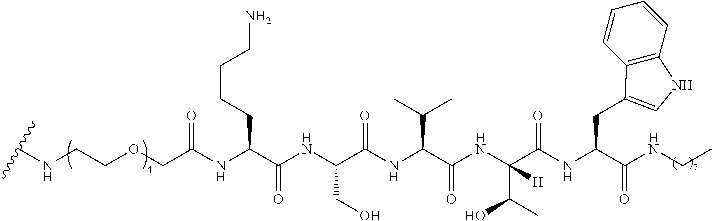 |

### Example 32: Preparation of Compounds TBK1-Diglycolate-(0-4)PEG (Target Protein Ligand-Diglycolate Linker)

The title compounds were prepared as described below. Preparation of Compound 3 in the scheme below is described in Example 10, above.

### Part I-Preparation of Compound TBK1-Diglycolate-0PEG

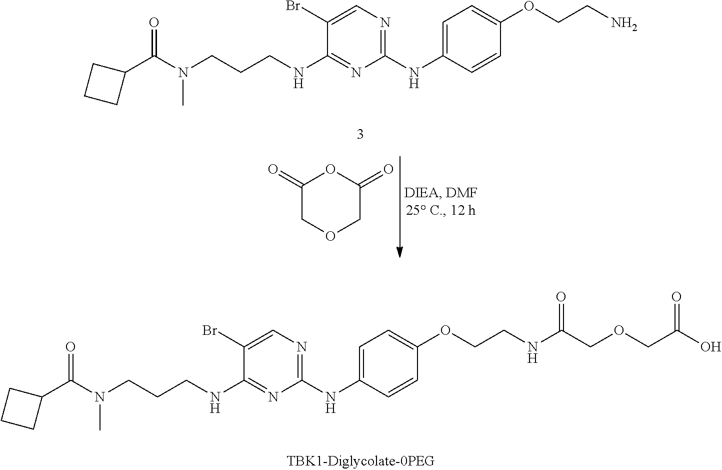 [SMILES: CN(CCCNc1nc(Nc2ccc(OCCN)cc2)ncc1Br)C(=O)C1CCC1 ; O=C1COCC(=O)O1 ; CN(CCCNc1nc(Nc2ccc(OCCNC(=O)COCC(=O)O)cc2)ncc1Br)C(=O)C1CCC1]

To a solution of N-(3-((2-((4-(2-aminoethoxy)phenyl)amino)-5-bromopyrimidin-4-yl)amino)propyl)-N-methylcyclobutanecarboxamide (Compound 3, 1.1 g, 2.2 mmol, 1 equiv, HCl salt) and DIEA (692 mg, 5.35 mmol, 2.5 equiv) in DMF (3 mL) was added 1,4-dioxane-2,6-dione (298 mg, 2.6 mmol, 1.2 equiv), and the mixture was stirred at 25° C. for 12 h. The mixture was neutralized with TFA, and the product was purified by prep-HPLC on a Phenomenex Luna C18 150×40 mm×15 um column using TFA modified water as mobile phase to afford compound TBK1-Diglycolate-0PEG (0.5 g, 39% yield). ¹H NMR (CD₃OD, 400 MHz) δ 7.90-7.93 (m, 1H), 7.36-7.42 (m, 2H), 6.98-7.05 (m, 2H), 4.18 (s, 2H), 4.05-4.15 (m, 4H), 3.64-3.68 (m, 2H), 3.47-3.49 (m, 2H), 3.38-3.41 (m, 2H), 3.25-3.31 (m, 1H), 2.83-2.92 (m, 3H), 2.20-2.25 (m, 3H), 1.75-1.85 (m, 5H). LC-MS: MS (ES⁺): RT=0.657 min, m/z=595.2 [M+H⁺].

### Part II-Preparation of Compound TBK1-Diglycolate-1PEG

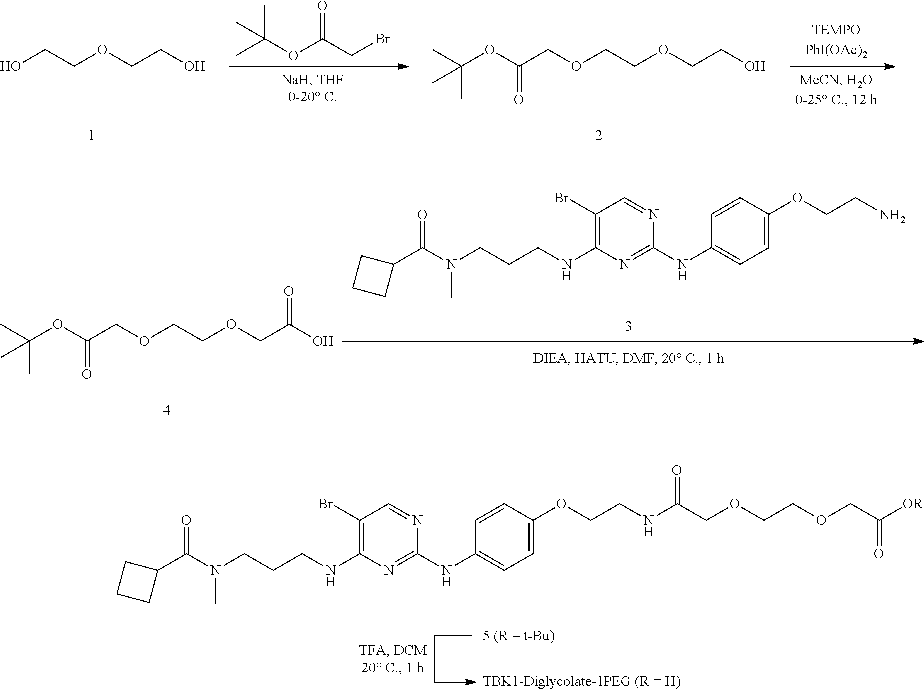

To a solution of compound 1 (80 g, 754 mmol, 72 mL, 1 equiv) in THF (800 mL) was added NaH (22.6 g, 565 mmol, 60% purity, 0.75 equiv) at 0° C. and the mixture was stirred at 20° C. for 1 h. tButyl 2-bromoacetate (147 g, 754 mmol, 111 mL, 1 equiv) was added at 0° C. and the mixture was stirred at 20° C. for another 1 h. The mixture was quenched with aq.
NH₄Cl (1000 mL) and extracted with EtOAc (3×1000 mL). The combined organic layers were washed with brine (500 mL), dried over anhydrous sodium sulfate, filtered, and concentrated. The residue was purified by column chromatography (SiO₂, Petroleum ether:Ethyl acetate=10:1 to 1:1) to afford compound 2 (23.4 g, 14% yield) as a colorless oil. ¹H NMR (CDCl₃, 400 MHz) δ 4.02 (s, 2H), 3.69-3.76 (m, 6H), 3.59-3.65 (m, 2H), 1.48 (s, 9H).
To a solution of compound 2 (5 g, 22.7 mmol, 1 equiv) in CH₃CN (20 mL) and H₂O (20 mL) were added TEMPO (785 mg, 5.0 mmol, 0.22 equiv) and (diacetoxyiodo)benzene (16 g, 50 mmol, 2.2 equiv) at 0° C., and then the mixture was stirred at 25° C. for 12 h. The mixture was diluted with water (200 mL) and extracted with EtOAc (3×300 mL). The combined organic layers were washed with brine (200 mL), dried over anhydrous sodium sulfate, filtered, and concentrated. The residue was purified by column chromatography (SiO₂, Petroleum ether:Ethyl acetate=20:1 to 1:1) to afford compound 4 (2.2 g, 41% yield) as a yellow oil. ¹H NMR (CDCl₃, 400 MHz) δ 8.40-8.30 (m, 1H), 4.18-4.23 (m, 2H), 4.00-4.06 (m, 2H), 3.81-3.71 (m, 4H), 1.48 (s, 9H).
To a solution of compound 3 (1 g, 2.0 mmol, 1 equiv, HCl salt) and compound 4 (456 mg, 2.0 mmol, 1 equiv) in DMF (10 mL) were added DIEA (754 mg, 6.0 mmol, 3 equiv) and HATU (888 mg, 2.4 mmol, 1.2 equiv). The mixture was stirred at 20° C. for 1 h and then concentrated. The residue was purified by prep-HPLC (column: Waters Xbridge C18 150*50 mm*10 um; mobile phase: [water (10 mM NH₄HCO₃)-ACN]; B %: 37%-67%, 11.5 min) to afford compound 5 (850 mg, 63% yield) as a yellow oil. ¹H NMR (CD₃OD, 400 MHz) δ 7.94-7.83 (m, 1H), 7.53-7.48 (m, 2H), 6.95-6.89 (m, 2H), 4.09 (t, J=5.63 Hz, 2H), 4.04 (s, 4H), 3.71 (s, 4H), 3.68-3.60 (m, 2H), 3.39-3.53 (m, 4H), 3.29-3.25 (m, 1H), 2.93 (s, 2H), 2.87 (s, 1H), 2.30-2.18 (m, 3H), 2.05-1.77 (m, 5H), 1.48 (s, 9H).
To a solution of compound 5 (850 mg, 1.2 mmol, 1 equiv) in DCM (6 mL) was added TFA (15.4 g, 135 mmol, 110 equiv). The mixture was stirred at 20° C. for 1 h and then concentrated to afford compound TBK1-Diglycolate-1PEG (780 mg, crude) as a yellow oil.

### Part III-Preparation of Compounds TBK1-Diglycolate-(2-4)PEG

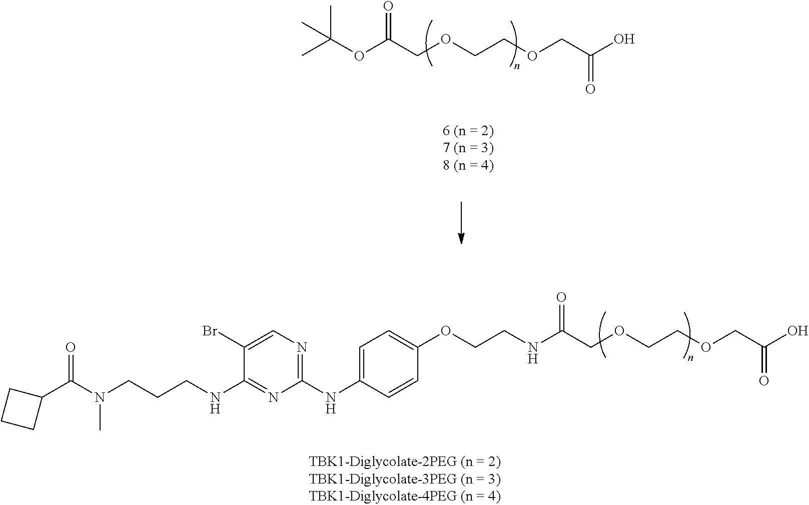

Compounds TBK1-Diglycolate-(2-4)PEG were prepared from Compound 6, 7, or 8, each of which can be prepared according to known procedures, and Compound 3, using similar procedures as described in Part II for synthesis of TBK1-Diglycolate-1PEG from Compounds 3 and 4.

### Example 33: Preparation of Protected Compound KRVHF-NMe (Protein Phosphatase Ligand)

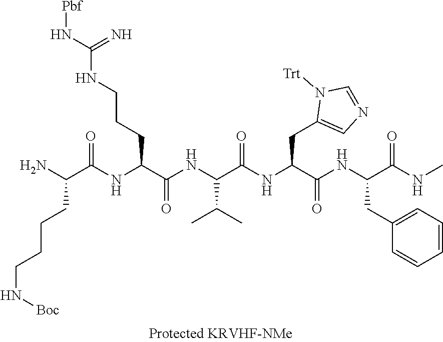

### Part I-Solid-Phase Synthesis of Protected Compound Fmoc-KRVHF-OH

Protected compound Fmoc-KRVHF-OH was synthesized via solid phase peptide synthesis and resin cleavage, according to the general synthetic procedures described in Example 2, above, and depicted in the scheme below. Protected compound Fmoc-KRVHF-OH was characterized by LC-MS: MS (ES⁺): RT=1.060 min, m/z=1503.7 [M+H⁺].

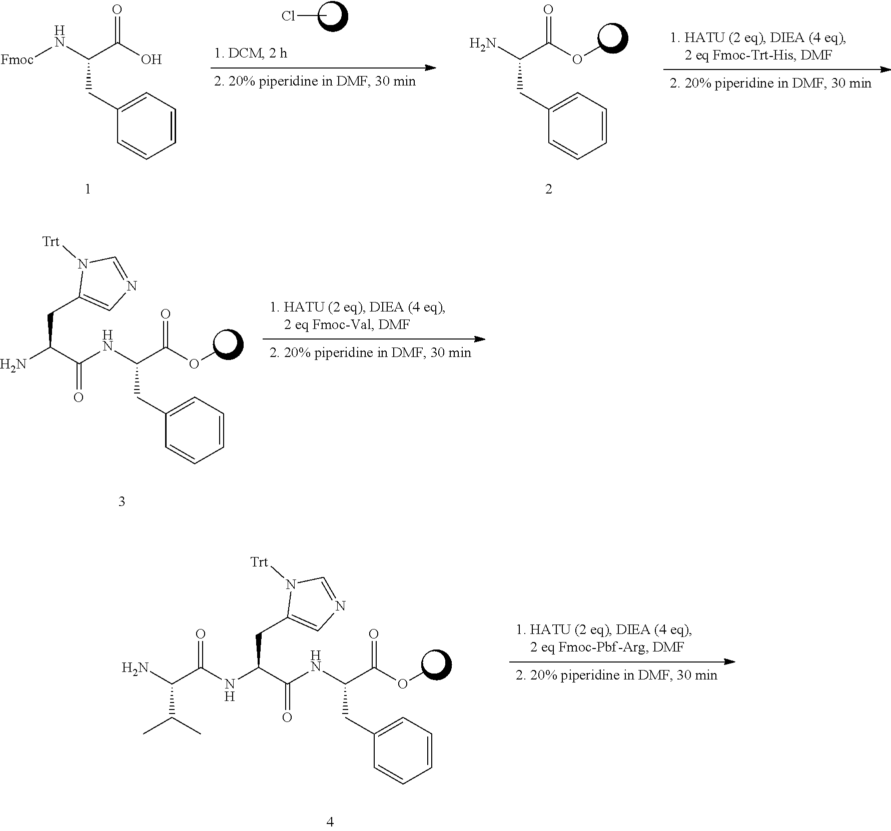

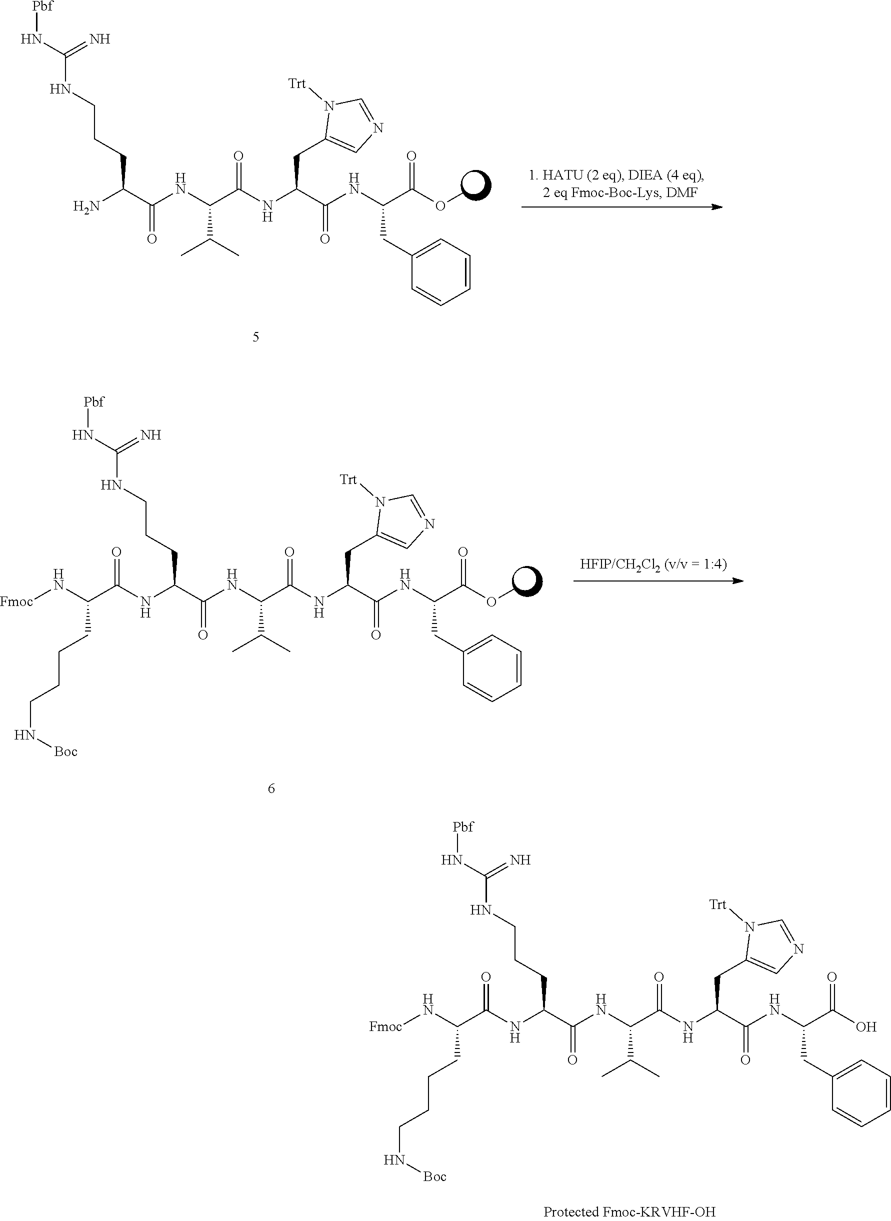

### Part II-Preparation of Protected Compound KRVHF-NMe

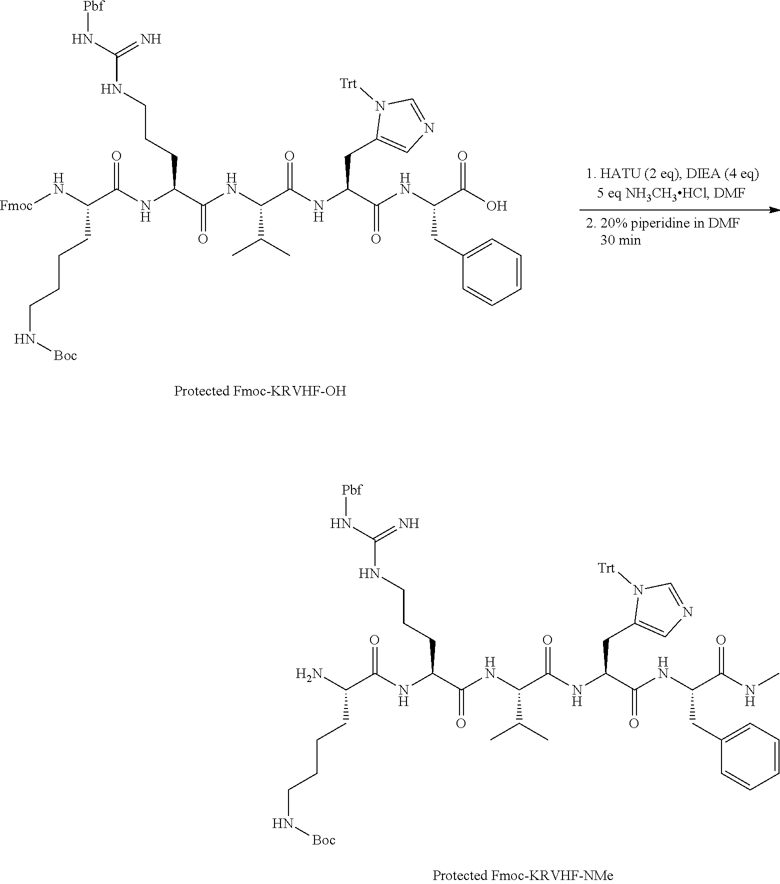

To a solution of protected compound Fmoc-KRVHF-OH (5.0 g, 3.3 mmol, 1.0 equiv), methanamine (1.1 g, 16.6 mmol, 5.0 equiv, HCl salt), and DIEA (860 mg, 6.7 mmol, 1.2 mL, 2.0 equiv) in DMF (20 mL) was added HATU (1.5 g, 4.0 mmol, 1.2 equiv). The mixture was stirred at 20° C. for 12 h. Then piperidine (3.5 g, 40.5 mmol, 4.0 mL, 12.3 equiv) was added and the mixture was stirred at 20° C. for 0.5 h. The solvent was removed and the residue was purified by prep-HPLC (column: Phenomenex Luna C18 250×50 mm×10 um; mobile phase: [water(0.1% TFA)-ACN]; B %: 30%-60%, 20 min) to give protected compound KRVHF-NMe (1.5 g, 35% yield) as a white solid. LC-MS: MS (ES⁺): RT=0.876 min, m/z=1293.6 [M+H⁺].

### Example 34: Preparation of Product Amide Compounds from Carboxylic Acid-Containing Target Protein Ligand-Diglycolate Linkers and Amine-Containing Protein Phosphatase Ligands

Product amide compounds in Table 22 below were prepared from carboxylic acid-containing target protein ligand-diglycolate linkers and amine-containing protein phosphatase ligands according to the general synthetic procedures below, as described and depicted in the schemes.

### Part I-General Procedure for Amide Coupling and Deprotection

To a solution of carboxylic acid-containing target protein ligand-diglycolate linker (˜135 μmol, 1.0 equiv) and amine-containing protein phosphatase ligand (˜1.0 equiv) in DMF (˜1 mL) was added HATU (˜1.2 equiv) and DIEA (˜3.0 equiv). The mixture was stirred at 25° C. for 1 h. The mixture was filtered and purified by prep-HPLC to give the protected amide compound.
A mixture of protected amide compound (˜100 μmol, 1.0 equiv) in H₂O (0.05 mL) and TFA (2 mL) was stirred at 25° C. for 2 h. The mixture was concentrated to give a residue. The residue was purified by prep-HPLC to afford the product amide compound.

### Preparation of Compound I-562

The following scheme depicts the coupling procedure for carboxylic acid-containing target protein ligand-diglycolate linker TBK1-Diglycolate-0PEG and protected amine-containing protein phosphatase ligand KRVHF-NMe to afford protected amide compound 1, and its subsequent deprotection to afford Compound I-562, according to the procedures described above.

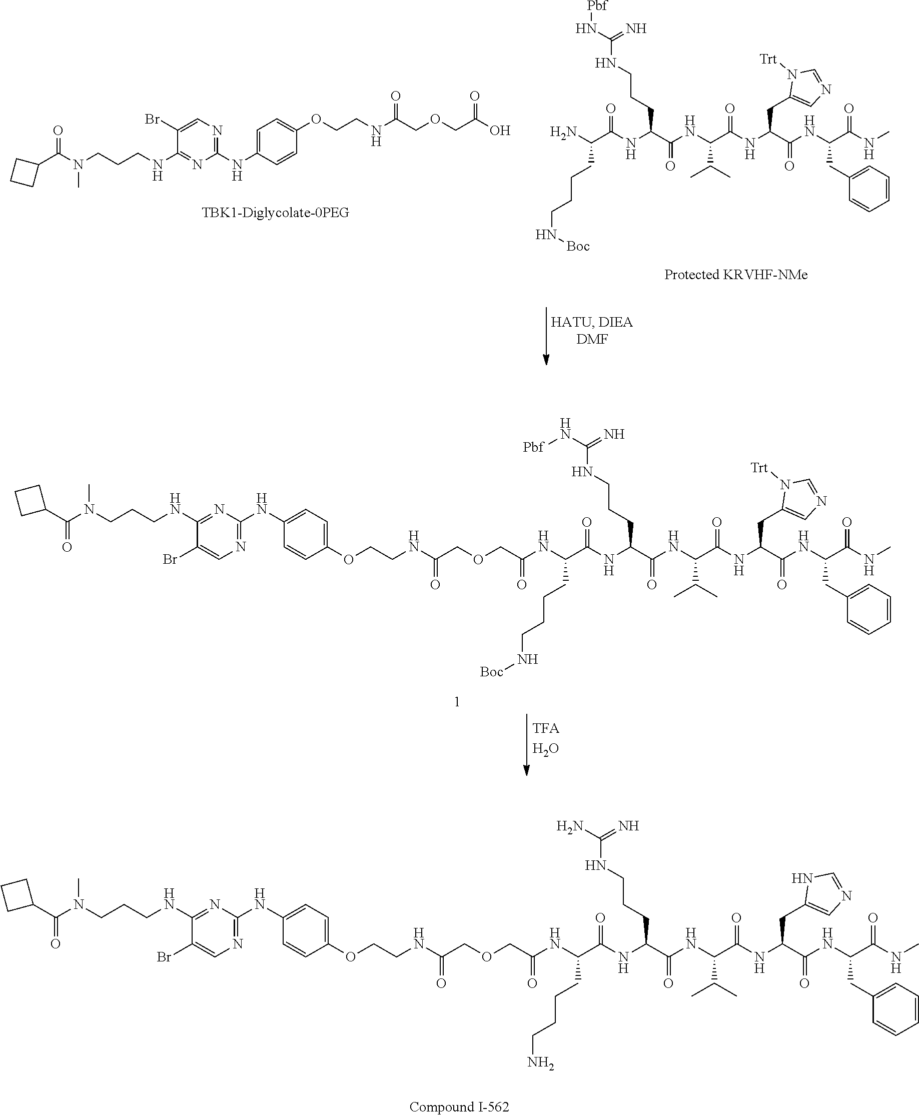

### Physical Characterization Data for Compound 1 and Compound I-562:

Compound 1 (200 mg, 79% yield) was isolated as a gray solid following purification by prep-HPLC (column: Waters Xbridge 150*25 mm*5 um; mobile phase: [water(10 mM NH₄HCO₃)-ACN]; B %: 61%-91%, 9 min). LC-MS: MS (ES⁺): RT=1.193 min, m/z=1869.7 [M+H⁺].
Compound I-562 (29 mg, 19% yield) was isolated as the HCl salt as a white solid following purification by prep-HPLC (column: Phenomenex Luna C18 150*25 mm*10 μm; mobile phase: [water(0.05% HCl)-ACN]; B %: 1%-35%, 10 min). ¹H NMR (CD₃OD, 400 MHz) δ 8.84 (s, 1H), 7.90-8.10 (m, 1H), 7.30-7.40 (m, 3H), 7.20-7.30 (m, 5H), 7.03-7.06 (m, 2H), 4.65-4.70 (m, 1H), 4.40-4.50 (m, 3H), 4.10-4.20 (m, 6H), 4.06-4.09 (m, 1H), 3.65-3.69 (m, 2H), 3.50-3.60 (m, 2H), 3.41-3.45 (m, 2H), 3.00-3.30 (m, 6H), 2.90-3.00 (m, 5H), 2.82 (s, 1H), 2.68 (s, 3H), 1.50-2.30 (m, 19H), 0.80-1.00 (m, 6H); LC-MS: MS (ES⁺): RT=2.103 min, m/z=637.3 [M/2+H⁺], 1272.7 [M+H⁺].

### Preparation of Compound I-563

Compound I-563 was prepared from protein ligand-diglycolate linker TBK1-Diglycolate-1PEG and protected amine-containing protein phosphatase ligand KRVHF-NMe according to the general procedures described above.

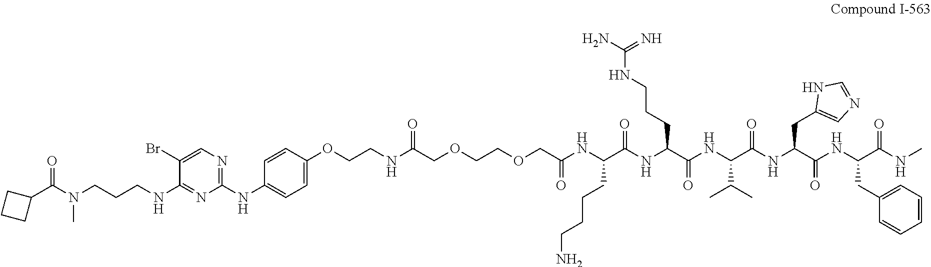 [SMILES: CNC(=O)[C@H](Cc1ccccc1)NC(=O)[C@H](Cc1cnc[nH]1)NC(=O)[C@@H](NC(=O)[C@H](CCCNC(=N)N)NC(=O)[C@H](CCCCN)NC(=O)COCCOCC(=O)NCCOc1ccc(Nc2ncc(Br)c(NCCCN(C)C(=O)C3CCC3)n2)cc1)C(C)C]

### Physical Characterization Data for Compound I-563:

Compound I-563 (9 mg, 19% yield) was isolated as the HCl salt as a white solid following purification by prep-HPLC (column: Phenomenex Luna C18 150*25 mm*10 μm; mobile phase: [water(0.1% HCl)-ACN]; B %: 4%-34%, 10 min). ¹H NMR (CD₃OD, 400 MHz) δ 8.86 (s, 1H), 8.07-7.89 (m, 1H), 7.43-7.35 (m, 3H), 7.31-7.22 (m, 5H), 7.07-7.05 (m, 2H), 4.71 (s, 1H), 4.55-4.42 (m, 3H), 4.16-4.08 (m, 7H), 3.80-3.67 (m, 6H), 3.58-3.40 (m, 4H), 3.26-3.18 (m, 4H), 3.13-3.05 (m, 1H), 3.00-2.93 (m, 5H), 2.84 (s, 1H), 2.70 (s, 3H), 2.28-2.20 (m, 3H), 2.12-1.42 (m, 17H), 0.96-0.87 (m, 6H); LC-MS: MS (ES⁺): RT=2.54 min, m/z=660.3 [M/2+H⁺], 1319.7 [M+H⁺].

### Part II-Exemplary Amide Compounds Prepared According to the General Procedures

The product amide compounds described in Table 22 below were prepared from carboxylic acid-containing target protein ligand-diglycolate linkers and amine-containing protein phosphatase ligands according to the procedures described in Part I. Chemical structures for abbreviations used in the description of product amide compounds in Table 22 are provided in Table 23 below.

TABLE 22
Exemplary Product Amide Compounds

| | | | Protected Protein | | | | |
|---|---|---|---|---|---|---|---|
| | | Target Protein | Phosphatase | | | | |
| | | Ligand-Diglycolate | Ligand Used to | | | | |
| Com- | | Linker Used to Prepare | Prepare Product | | LC/MS | | |
| pound | Product Amide | Product Amide | Amide | | MH + | | |
| No. | Compound | Compound | Compound | MW | (obs) | RT | Method |
| I-562 | (TBK1-Diglycolate-0PEG)- | TBK1- | KRVHF-NMe | 1274.3 | 1274.3 | 2.10 | 01 |
| | (KRVHF-NMe) | Diglycolate-0PEG | | | | | |
| I-563 | (TBK1-Diglycolate-1PEG)- | TBK1- | KRVHF-NMe | 1318.4 | 1320.6 | 2.54 | 00 |
| | (KRVHF-NMe) | Diglycolate-1PEG | | | | | |
| I-564 | (TBK1-Diglycolate-2PEG)- | TBK1- | KRVHF-NMe | 1362.4 | 1363.6 | 2.58 | 00 |
| | (KRVHF-NMe) | Diglycolate-2PEG | | | | | |
| I-565 | (TBK1-Diglycolate-3PEG)- | TBK1- | KRVHF-NMe | 1406.5 | 704.3 | 2.60 | 00 |
| | (KRVHF-NMe) | Diglycolate-3PEG | | | (M/2 + H) | | |
| I-566 | (TBK1-Diglycolate-4PEG)- | TBK1- | KRVHF-NMe | 1450.5 | 726.3 | 2.62 | 00 |
| | (KRVHF-NMe) | Diglycolate-4PEG | | | (M/2 + H) | | |

TABLE 23
Chemical Structures Corresponding to Abbreviations Used in the Description of the Product Amide Compounds

| Abbreviation | Chemical Structure |
|---|---|
| (TBK1- Diglycolate- 0PEG)- | 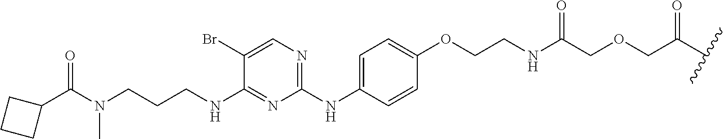 [SMILES: *C(=O)COCC(=O)NCCOc1ccc(Nc2ncc(Br)c(NCCCN(C)C(=O)C3CCC3)n2)cc1] |
| (TBK1- Diglycolate- 1PEG)- | 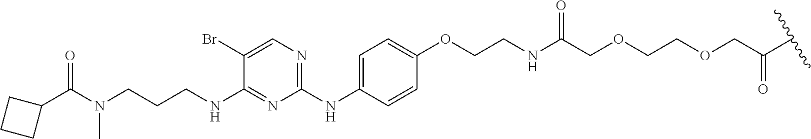 [SMILES: *C(=O)COCCOCC(=O)NCCOc1ccc(Nc2ncc(Br)c(NCCCN(C)C(=O)C3CCC3)n2)cc1] |
| (TBK1- Diglycolate- 2PEG)- | 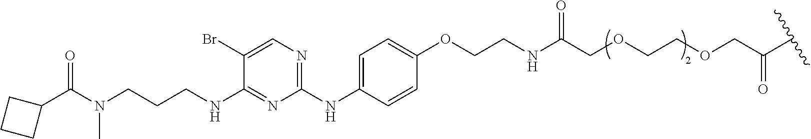 |
| (TBK1- Diglycolate- 3PEG)- | 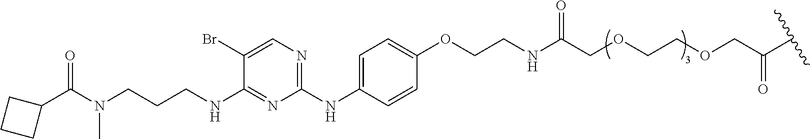 |
| (TBK1- Diglycolate- 4PEG)- | 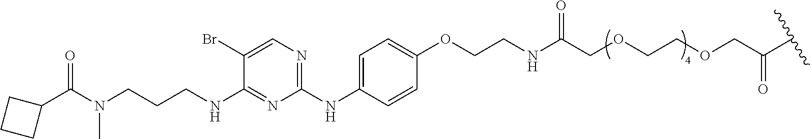 |
| -(KRVHF- NMe) | 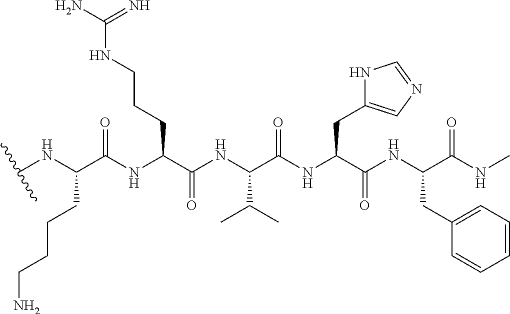 [SMILES: *N[C@@H](CCCCN)C(=O)N[C@@H](CCCNC(=N)N)C(=O)N[C@H](C(=O)N[C@@H](Cc1cnc[nH]1)C(=O)N[C@@H](Cc1ccccc1)C(=O)NC)C(C)C] |

### Example 35: Biological Assay for Dephosphorylation of pAKT Serine-473 and Threonine-308

Exemplary compounds from the above Examples were tested for ability to cause dephosphorylation of pAKT Serine-473 and Threonine-308. Experimental procedures and results are provided below.

### Part I-Experimental Procedure for Western Blot Assay

Dephosphorylation of p-TBK1 was measured according to the following procotol: panc02.13 or THP-1 cells were purchased from ATCC and cultured in RPMI-1640 media, supplemented with 10% FBS. Poly I:C agonist (Invivogen) was added to the cells 1 hr before treatment with test compound. Vehicle and test compound treatments (25 μM, and 2.5 μM) were performed in 12-well plates for 2 hours. Cells were harvested and lysed in RIPA buffer (50 mM Tris pH8, 150 mM NaCl, 1% Tx-100, 0.1% SDS and 0.5% sodium deoxycholate) supplemented with protease and phosphatase inhibitors. Lysates were clarified at 16,000 g for 10 minutes and supernatants were separated by SDS-PAGE. Immunoblotting was performed using standard protocols, with antibodies for p-TBK1 (Cell Signaling, #5483) and total TBK1 (Cell Signaling, #38066, #51877 or #3504). The signal intensity for bands was imaged on a LiCor Odyssey imager.

### Part II-Experimental Procedure for Surefire Assay

Phosphorylation of S473 and T308 in AKT was measured using the following protocol. PC3 or HEK-293 cells were cultured as suggested by ATCC. Vehicle and test compound treatments (25 μM, and 2.5 μM) were performed in 96-well plates for 2 hours. Media was aspirated and the cells were lysed in 70 μL of lysis buffer (Perkin Elmer SureFire). Plates were agitated gently for 10 minutes at 400 rpm.

### Add Acceptor Mix to Lysates:

Transfer 10 μL of lysate to white 384 well Optiplate (Perkin-Elmer #6007290)
Add certain volumes of PC3 and HEK293 lysates to appropriate wells
Add 10 μL positive control to appropriate wells
Add 5 μL of appropriate Acceptor mix to each well (prepare as suggested in SureFire protocol)
Incubate for 1 hour at RT

### Add Donor Mix:

Donor mix was prepared within 15 minutes before Acceptor mix incubation was done; the Donor mix was used within 30 minutes of mixing.
Steps performed on Donor beads were to be performed under low light conditions
Vortex Donor beads (to get beads into solution) and spin down briefly to collect solution at bottom of tube
Add 5 μL of appropriate mix to appropriate wells of 384 well Optiplate and cover with an aluminum plate seal
Incubate for 1 hour at RT
Spin down plates if necessary

### Read Plate:

Optimized protocol is called Alphalisa
excitation is 680 for donor bead
emission is 615 and 545 for pAKT and total AKT respectively
Mirror need is AlphaPlex Dual Tb-Eu (#2102-5900)
Filter needed is AlphaPlex Tb (#2100-5930)
If insufficient signal was observed, then plate was covered and kept in dark at RT O/N and re-read next day.

### Part III-Results

Experimental results are provided in the table below. The symbol “++++” indicates less than 25%. The symbol “+++” indicates a number in the range of 25% to 50%. The symbol “++” indicates a number in the range of greater than 50% to 75%. The symbol “+” indicates a number in the range of greater than 75% to 99%. The symbol “*” indicates a number greater than 99%. The symbol “N/A” indicates that no data was available.

TABLE 24
Results of Assays for Dephosphorylation of pAKT Serine-473
and Threonine 308

| | Western Blot Assay | | SureFire Assay | | SureFire Assay | |
|---|---|---|---|---|---|---|
| | (% pAKT S473) | | (% pAKT S473) | | (% pAKT T308) | |
| | 2.5 μM | 25 μM | 2.5 μM | 25 μM | 2.5 μM | 25 μM |
| Com- | Test | Test | Test | Test | Test | Test |
| pound | Com- | Com- | Com- | Com- | Com- | Com- |
| No. | pound | pound | pound | pound | pound | pound |
| I-40 | + | ++ | + | ++++ | N/A | ++++ |
| I-41 | ++ | ++++ | +++ | ++++ | N/A | ++++ |
| I-42 | ++ | ++++ | ++++ | ++++ | N/A | ++++ |
| I-43 | ++ | +++ | +++ | ++++ | N/A | ++++ |
| I-44 | ++ | ++++ | ++++ | ++++ | N/A | ++++ |
| I-45 | * | + | N/A | +++ | N/A | +++ |
| I-46 | * | + | N/A | +++ | N/A | +++ |
| I-47 | * | + | N/A | +++ | N/A | +++ |
| I-48 | * | ++ | N/A | ++++ | N/A | ++++ |
| I-49 | + | ++ | N/A | ++++ | N/A | ++++ |
| I-50 | ++ | ++ | ++ | +++ | N/A | +++ |
| I-51 | +++ | ++++ | +++ | ++++ | N/A | ++++ |
| I-52 | +++ | ++++ | ++++ | ++++ | N/A | ++++ |
| I-53 | +++ | ++++ | ++++ | ++++ | N/A | ++++ |
| I-54 | +++ | ++++ | ++++ | ++++ | N/A | ++++ |
| I-146 | ++ | ++ | N/A | +++ | N/A | +++ |
| I-149 | + | +++ | N/A | +++ | N/A | +++ |
| I-152 | + | ++ | N/A | +++ | N/A | +++ |
| I-155 | + | +++ | N/A | ++++ | N/A | ++++ |
| I-157 | + | ++ | N/A | +++ | N/A | +++ |
| I-147 | + | + | N/A | +++ | N/A | +++ |
| I-156 | + | +++ | N/A | +++ | N/A | +++ |
| I-158 | + | +++ | N/A | ++++ | N/A | ++++ |
| I-150 | * | ++ | N/A | ++ | N/A | ++ |
| I-153 | ++ | +++ | N/A | +++ | N/A | +++ |
| I-148 | + | ++ | N/A | ++ | N/A | +++ |
| I-151 | ++ | +++ | N/A | +++ | N/A | +++ |
| I-154 | + | ++ | N/A | ++ | N/A | ++ |
| I-76 | + | ++ | N/A | + | N/A | N/A |
| I-77 | + | + | N/A | + | N/A | N/A |
| I-78 | + | + | N/A | ++ | N/A | N/A |
| I-75 | + | + | N/A | + | N/A | N/A |
| I-79 | ++ | + | N/A | + | N/A | N/A |
| I-81 | * | + | N/A | * | N/A | N/A |
| I-82 | * | + | N/A | * | N/A | N/A |
| I-83 | + | + | N/A | * | N/A | N/A |
| I-80 | * | + | N/A | + | N/A | N/A |
| I-84 | + | + | N/A | * | N/A | N/A |
| I-86 | + | + | N/A | + | N/A | N/A |
| I-87 | + | + | N/A | + | N/A | N/A |
| I-89 | + | ++ | N/A | + | N/A | N/A |
| I-88 | + | + | N/A | + | N/A | N/A |
| I-159 | + | ++ | N/A | +++ | N/A | N/A |
| I-162 | + | ++ | N/A | ++ | N/A | N/A |
| I-171 | ++ | + | N/A | ++ | N/A | N/A |
| I-165 | + | + | N/A | ++ | N/A | N/A |
| I-168 | + | + | N/A | ++ | N/A | N/A |
| I-160 | + | * | N/A | + | N/A | N/A |
| I-163 | + | * | N/A | * | N/A | N/A |
| I-166 | + | N/A | N/A | * | N/A | N/A |
| I-169 | + | * | N/A | + | N/A | N/A |
| I-172 | + | + | N/A | + | N/A | N/A |
| I-170 | + | + | N/A | ++ | N/A | N/A |
| I-161 | + | + | N/A | ++ | N/A | N/A |
| I-164 | ++ | + | N/A | ++ | N/A | N/A |
| I-167 | ++ | + | N/A | ++ | N/A | N/A |
| I-173 | ++ | ++ | N/A | +++ | N/A | N/A |
| I-55 | + | + | N/A | + | N/A | ++ |
| I-56 | + | ++ | N/A | ++ | N/A | +++ |
| I-57 | + | ++ | N/A | ++ | N/A | ++ |
| I-58 | * | * | N/A | * | N/A | * |
| I-59 | + | ++ | N/A | +++ | N/A | +++ |
| I-63 | + | ++ | N/A | +++ | N/A | +++ |
| I-64 | + | ++ | N/A | +++ | N/A | +++ |
| I-61 | + | ++ | N/A | ++ | N/A | +++ |
| I-62 | + | ++ | N/A | ++ | N/A | ++ |
| I-60 | + | + | N/A | + | N/A | + |
| I-69 | N/A | N/A | ++ | +++ | +++ | ++ |
| I-65 | N/A | N/A | ++ | ++ | + | + |
| I-66 | N/A | N/A | ++ | ++ | * | * |
| I-67 | N/A | N/A | ++ | +++ | ++ | +++ |
| I-68 | N/A | N/A | +++ | ++++ | ++ | +++ |
| I-70 | + | + | N/A | + | N/A | + |
| I-71 | + | +++ | N/A | +++ | N/A | ++++ |
| I-72 | + | ++ | N/A | ++ | N/A | ++ |
| I-73 | + | ++ | N/A | +++ | N/A | +++ |
| I-74 | + | ++ | N/A | +++ | N/A | +++ |
| I-90 | + | + | N/A | ++ | N/A | N/A |
| I-92 | + | ++ | N/A | +++ | N/A | N/A |
| I-94 | * | + | N/A | +++ | N/A | N/A |
| I-93 | + | + | N/A | ++ | N/A | N/A |
| I-91 | + | + | N/A | ++ | N/A | N/A |
| I-97 | + | + | N/A | ++ | N/A | N/A |
| I-99 | + | * | N/A | ++ | N/A | N/A |
| I-96 | * | + | N/A | +++ | N/A | N/A |
| I-98 | + | + | N/A | +++ | N/A | N/A |
| I-95 | + | + | N/A | +++ | N/A | N/A |
| I-102 | N/A | N/A | N/A | +++ | N/A | N/A |
| I-103 | N/A | N/A | N/A | +++ | N/A | N/A |
| I-100 | N/A | N/A | N/A | ++ | N/A | N/A |
| I-104 | N/A | N/A | N/A | ++ | N/A | N/A |
| I-101 | N/A | N/A | N/A | +++ | N/A | N/A |
| I-105 | * | + | N/A | +++ | N/A | N/A |
| I-106 | + | ++ | N/A | ++++ | N/A | N/A |
| I-107 | + | ++ | N/A | ++ | N/A | N/A |
| I-108 | * | ++ | N/A | +++ | N/A | N/A |
| I-109 | + | ++ | N/A | +++ | N/A | N/A |

### Example 36: Biological Assay for Dephosphorylation of pTBK1 Serine172

Exemplary compounds from the above Examples were tested for ability to cause dephosphorylation of pTBK1 Serine172. Experimental procedures and results are provided below.

### Part I-Experimental Procedure

200,000 THP1 cells (ATCC TIB-202) were seeded into each well of a 96 well plate in RPMI culture medium (Gibco A10491) supplemented with 10% fetal bovine serum (Gibco A3160402), 100 unites/mL penicillin-streptomycin (Gibco 15140122), and 100 nM phorbol 12-myristate 13-acetate (PMA). Following 2 days of differentiation, culture medium was replaced with serum-free RPMI medium containing the indicated concentration of test compound and incubated for 1 hour at 37° C. Following incubation, the medium was replaced with RPMI containing the indicated concentration of test compound and 1 μg/mL lipopolysaccharide 0111:B4 (LPS) (Sigma L4391) and incubated for 1 hr at 37° C. After treatment, medium was removed and 50 μL of Alpha Surefire Ultra Lysis buffer (Perkin Elmer ALSO-LB-100 mL) was added to each well. Plates were then placed on a plate shaker at 400 RPM for 30 minutes. For detection, 16 μL of lysate was added to 4 uL of working antibody solution per the Cisbio protocol for homogeneous time resolved fluorescence (HTRF) detection of phospho TBK1 Serine 172 (Cisbio 64TBKPEG). Plates were incubated overnight at room temperature.
Fluorescence signal was detected using an EnVision 2105 plate reader (Perkin Elmer). The Europium conjugated anti-phospho TBK1 serine 172 antibody wavelength excited at 320 nm, direct emission of the europium conjugated antibody was detected with a 615 nm filter and the FRET signal to a d2 conjugated anti-TBK1 acceptor antibody was detected at 665 nm. FRET signal at 665 nm was normalized to incident light signal at 615 nm and multiplied by 10,000 per the Cisbio protocol. Basal signal from vehicle-treated cells was subtracted from signal measured in all other treatments. This background-subtracted signal was then normalized as a percent of the average signal of cells treated with LPS alone.

### Part II-Results

Experimental results are provided in the table below. The symbol “++++” indicates less than 25%. The symbol “+++” indicates a number in the range of 25% to 50%. The symbol “++” indicates a number in the range of greater than 50% to 75%. The symbol “+” indicates a number in the range of greater than 75% to 99%. The symbol “*” indicates a number greater than 99%.

The symbol “N/A” indicates that no data was available.

TABLE 25
Results of Assays for Dephosphorylation of pTBK Serine172

| | pTBK1 S172 | |
|---|---|---|
| | (% of LPS-induced signal) | |
| Compound | 2.5 μM Test | 25 μM Test |
| No. | Compound | Compound |
| I-110 | * | +++ |
| I-111 | * | ++ |
| I-112 | + | +++ |
| I-113 | + | +++ |
| I-114 | * | +++ |
| I-115 | + | ++++ |
| I-116 | * | +++ |
| I-117 | + | ++++ |
| I-118 | * | +++ |
| I-119 | * | +++ |
| I-120 | + | +++ |
| I-121 | * | ++ |
| I-122 | * | ++++ |
| I-123 | * | ++++ |
| I-124 | * | ++ |
| I-194 | * | * |
| I-191 | * | * |
| I-197 | * | ++ |
| I-192 | * | + |
| I-195 | * | ++ |
| I-198 | * | ++ |
| I-201 | * | ++ |
| I-190 | * | + |
| I-193 | + | ++ |
| I-196 | * | ++ |
| I-200 | * | * |
| I-203 | + | * |
| I-125 | ++ | ++ |
| I-126 | ++ | ++ |
| I-127 | ++ | +++ |
| I-128 | +++ | ++++ |
| I-129 | +++ | +++ |
| I-130 | + | * |
| I-131 | * | * |
| I-133 | * | * |
| I-132 | + | * |
| I-136 | ++++ | ++++ |
| I-138 | ++++ | ++++ |
| I-139 | ++++ | ++++ |
| I-140 | ++++ | ++++ |
| I-137 | ++++ | ++++ |
| I-134 | * | * |
| I-135 | * | * |

### Example 37: Biological Assay for Dephosphorylation of pTBK1 Serine172

Exemplary compounds from the above Examples were tested at multiple concentrations for ability to cause dephosphorylation of pTBK1 Serine172 in THP1 cells. Determination of percent dephosphorylation of pTBK1 Serine172 in THP1 cells was determined based on procedures described in Example 36, except using different concentrations of test compound. Based on the percent dephosphorylation observed at the various concentrations of test compound, a DP₅₀ value was determined (DP₅₀=concentration at which 50% dephosphorylation is achieved).
Results for exemplary compounds are provided in Table 26 below.

TABLE 26
Concentration-response Data for In-vitro Dephosphorylation of pTBK1 Serine172 in THP1 Cells

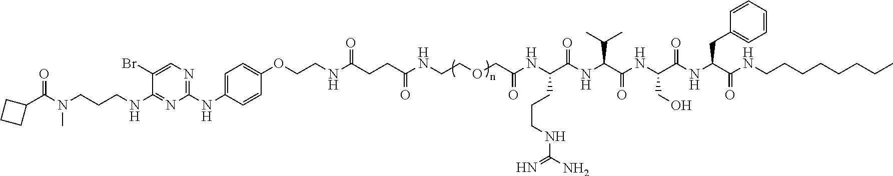

| Compound No. | Linker Length (n) | pTBK1 S172 DP₅₀ (μM) |
|---|---|---|
| I-190 | 0 | >225 |
| I-193 | 1 | >225 |
| I-196 | 2 | <65 |
| I-199 | 3 | <65 |
| I-202 | 4 | >225 |

### Enumerated Embodiments

The following exemplary embodiments are provided, the numbering of which is not to be construed as designating levels of importance.

Embodiment 1 provides a compound of formula (I), or a salt, solvate, prodrug, isotopically labelled derivative, stereoisomer, tautomer, or geometric isomer thereof, and any mixtures thereof:

(protein phosphatase ligand)-LINKER-(target protein ligand) (I); wherein the protein phosphatase ligand binds to a protein phosphatase, such that the protein phosphatase ligand does not significantly inhibit the phosphatase activity of the protein phosphatase; wherein the target protein ligand binds to a target protein; wherein the LINKER is selected such that it allows for the compound to bind simultaneously to the protein phosphatase and the target protein; wherein, when the compound is simultaneously bound to the protein phosphatase and the target protein, the protein phosphatase is capable of dephosphorylating the target protein.

Embodiment 2 provides the compound of Embodiment 1, wherein the protein phosphatase comprises at least one selected from the group consisting of protein phosphatase 1 (PP1), protein phosphatase 2A (PP2A), protein phosphatase 2B (PP2B), protein phosphatase 2C (PP2C), any of PTPRA through PTPRZ, and dual specific phosphatases DUSP1 through DUSP27.

Embodiment 3 provides the compound of Embodiment 1, wherein the protein phosphatase comprises at least one selected from the group consisting of CDC25A, CDC25B, CDC25C, ACP1, and Eya1 through Eya4.

Embodiment 4 provides the compound of any of Embodiments 1-2, wherein the protein phosphatase is PP1 and wherein the protein phosphatase ligand comprises the amino acid sequence Arg Val Xaa Phe (SEQ ID NO:1).

Embodiment 5 provides the compound of any of Embodiments 1-2 and 4, wherein the protein phosphatase ligand comprises the amino acid sequence selected from the group consisting of RRKRPKRKRKNARVTF(Xaa)EAAEII (SEQ ID NO:2) and RRKRPKRKRKNARVTFFEAAEII (SEQ ID NO:3).

Embodiment 6 provides the compound of any of Embodiments 1-2, wherein the protein phosphatase is PP2 and wherein the protein phosphatase ligand comprises the amino acid sequence Leu Ser Pro Ile Xaa Glu (SEQ ID NO:4).

Embodiment 7 provides the compound of any of Embodiments 1-2 and 6, wherein the protein phosphatase ligand comprises the amino acid sequence GLLSPIPERRRRRRRR (SEQ ID NO:5).

Embodiment 8 provides the compound of any of Embodiments 1-3, which comprises a compound of formula (II), or a salt, solvate, prodrug, isotopically labelled derivative, stereoisomer, tautomer, and/or geometric isomer thereof, and any mixtures thereof:

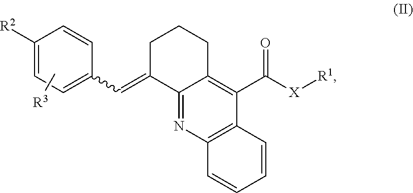

wherein: X is selected from the group consisting of a bond, —O—, —NH—, and —N(C₁-C₆ alkyl)-; one selected from the group consisting of R¹, R², and R³ is -LINKER-(target protein ligand); and the other two are independently selected from the group consisting of optionally substituted C₁-C₆ alkyl, —OH, optionally substituted C₁-C₆ alkoxy, —NH₂, —NH(optionally substituted C₁-C₆ alkyl), and —N(optionally substituted C₁-C₆ alkyl)(optionally substituted C₁-C₆ alkyl).

Embodiment 9 provides the compound of any of Embodiments 1-3, which comprises a compound of formula (III), or a salt, solvate, prodrug, isotopically labelled derivative, stereoisomer, tautomer, and/or geometric isomer thereof, and any mixtures thereof:

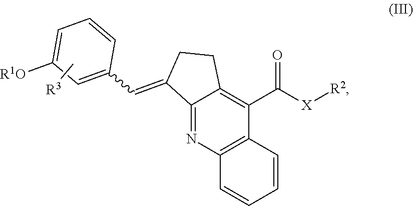

wherein: X is selected from the group consisting of a bond, —O—, —NH—, and —N(C₁-C₆ alkyl)-; one of the following applies: (i) R³ is -LINKER-(target protein ligand), and R¹ and R² are independently selected from the group consisting of H, optionally substituted C₁-C₆ alkyl, and optionally substituted C₃-C₈ cycloalkyl; (ii) one selected from the group consisting of R¹ and R² is -LINKER-(target protein ligand), and the other is selected from the group consisting of H, optionally substituted C₁-C₆ alkyl, and optionally substituted C₃-C₈ cycloalkyl; and R³ is selected from the group consisting of optionally substituted C₁-C₆ alkyl, —OH, optionally substituted C₁-C₆ alkoxy, —NH₂, —NH(optionally substituted C₁-C₆ alkyl), and —N(optionally substituted C₁-C₆ alkyl)(optionally substituted C₁-C₆ alkyl).

Embodiment 10 provides the compound of any of Embodiments 1-3, which comprises a compound of formula (IV), or a salt, solvate, prodrug, isotopically labelled derivative, stereoisomer, tautomer, and/or geometric isomer thereof, and any mixtures thereof:

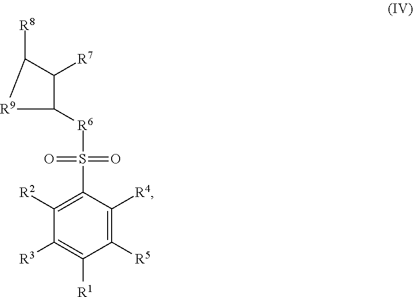

wherein: R¹ is selected from the group consisting of H, C₁-C₆ haloalkyl, C₁-C₆ haloalkoxy, and -LINKER-(target protein ligand); each one of R², R³, R⁴, and R⁵ is independently selected from the group consisting of H and -LINKER-(target protein ligand); R⁶ is selected from the group consisting of —CH₂—, —CH(LINKER-target protein ligand)-, —NH—, and —N(LINKER-target protein ligand)-; R⁷ is selected from the group consisting of H and OH; R⁸ is selected from the group consisting of

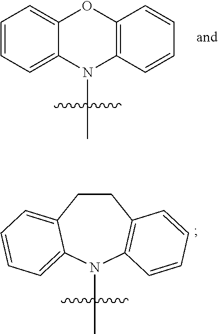 [SMILES: *N1c2ccccc2Oc2ccccc21 ; *N1c2ccccc2CCc2ccccc21]

R⁹ is selected from the group consisting of null (absent), —CH₂—, —CH₂CH₂—, and —CH₂CH₂CH₂—; with the proviso that only one of R¹-R⁶ comprises -LINKER-(target protein ligand).

Embodiment 11 provides the compound of any of Embodiments 1-3, which comprises a compound of formula (V), or a salt, solvate, prodrug, isotopically labelled derivative, stereoisomer, tautomer, and/or geometric isomer thereof, and any mixtures thereof:

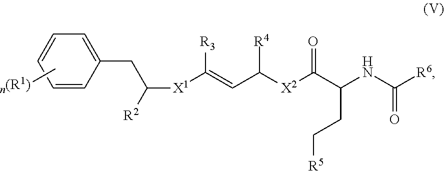

wherein: each occurrence of R¹, R², R³, R⁴, R⁵, and R⁶ is independently selected from the group consisting of H, -LINKER-(target protein ligand), halogen, NO₂, C₁-C₆ alkyl, C₁-C₆ haloalkyl, C₃-C₈ cycloalkyl, C₂-C₆ alkenyl, C₂-C₆ alkynyl, C₁-C₆ alkoxy, optionally substituted aryl, and optionally substituted heteroaryl, wherein the alkyl, cycloalkyl, alkenyl, or alkynyl is optionally independently substituted with at least one selected from the group consisting of hydroxyl-OR′, NR′R′, amide, —C(═O)OR′, guanidino, —SR′, halogen, C₁-C₆ alkyl, C₃-C₈ cycloalkyl, C₂-C₆ alkenyl, C₁-C₆ alkyl, alkoxy, and heteroaryl, wherein each occurrence of R′ is independently H or C₁-C₆ alkyl; n is 0, 1, 2, 3, 4, or 5; X¹ and X² are independently selected from group consisting of —NH—, —O—, C₁-C₆ alkylene, C₃-C₈ cycloalkyene, C₂-C₆ alkenylene, C₂-C₆ alkynylene, C₁-C₆ alkoxydiyl, optionally substituted arylene, and optionally substituted heteroarylene, wherein the alkylene, cycloalkylene, alkenylene, or alkynylene is optionally independently substituted with at least one selected from the group consisting of hydroxyl-OR′, NR′R′, amide, —C(═O)OR′, guanidino, —SR′, halogen, C₁-C₆ alkyl, C₃-C₈ cycloalkyl, C₂-C₆ alkenyl, C₁-C₆ alkyl, alkoxy, and heteroaryl, wherein each occurrence of R′ is independently H or C₁-C₆ alkyl; with the proviso that only one of R¹-R⁶ comprises -LINKER-(target protein ligand).

Embodiment 12 provides the compound of any of Embodiments 1-11, wherein the compound is a compound of formula (I) or a salt thereof.

Embodiment 13 provides a compound of formula (I-A), or a salt or solvate thereof:
(protein phosphatase ligand)-LINKER-(target protein ligand) (I-A);
   wherein: the protein phosphatase ligand binds to a protein phosphatase; the target protein ligand binds to a target protein; the LINKER is a bond or a group that allows the compound to bind to the protein phosphatase and the target protein.

Embodiment 14 provides the compound of Embodiment 13, wherein the protein phosphatase ligand does not significantly inhibit phosphatase activity of the protein phosphatase.

Embodiment 15 provides the compound of any of Embodiments 1-14, wherein the protein phosphatase ligand binds to protein phosphatase 1 (PP1).

Embodiment 16 provides the compound of any of Embodiments 1-15, wherein the protein phosphatase ligand binds protein phosphatase 2A (PP2A), protein phosphatase 2B (PP2B), or protein phosphatase 2C (PP2C).

Embodiment 17 provides the compound of any of Embodiments 1-16, wherein the protein phosphatase comprises at least one selected from the group consisting of CDC25A, CDC25B, CDC25C, ACP1, and Eya1 through Eya4.

Embodiment 18 provides the compound of any of Embodiments 1-17, wherein the protein phosphatase ligand component of Formula (I) has the following formula:

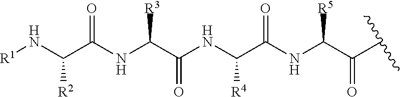

wherein: R¹ is hydrogen or an optionally substituted group selected from —C(═O)(C₁-C₈ alkyl), —C(═O)(C₃-C₈ cycloalkyl), —C(═O)(C₀-C₃ alkylene)-aryl, and C₁-C₈ alkyl; R² is optionally substituted —(C₁-C₈ alkylene)-N(H)—C(═NH)NH₂; R³ is optionally substituted C₁-C₈ alkyl; R⁴ is optionally substituted C₁-C₈ hydroxyalkyl; and R⁵ is optionally substituted —(C₀-C₃ alkylene)-aryl or optionally substituted —(C₀-C₃ alkylene)-heteroaryl.

Embodiment 19 provides the compound of any of Embodiments 1-17, wherein the protein phosphatase ligand component of Formula (I) has the following formula:

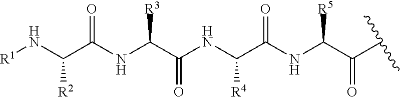

wherein: R¹ is hydrogen or —C(═O)(C₁-C₈ alkyl); R is —(C₁-C₈ alkylene)-N(H)—C(═NH)NH₂; R³ is C₁-C₈ alkyl; R⁴ is C₁-C₈ hydroxyalkyl; and R⁵ is —(C₀-C₃ alkylene)-aryl.

Embodiment 20 provides the compound of any of Embodiments 1-17, wherein the protein phosphatase ligand component of Formula (I) has the following formula:

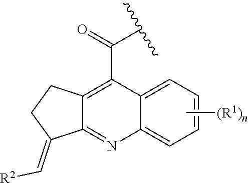

wherein: R¹ represents independently for each occurrence halogen, C₁-C₆ alkyl, C₁-C₆ haloalkyl, C₃-C₆ cycloalkyl, hydroxyl, C₁-C₆ alkoxy, or cyano; R² is optionally substituted —(C₀-C₃ alkylene)-aryl or optionally substituted —(C₀-C₃ alkylene)-heteroaryl; and n is 0, 1, 2, or 3.

Embodiment 21 provides the compound of any of Embodiments 1-17, wherein the protein phosphatase ligand component of Formula (I) has the following formula:

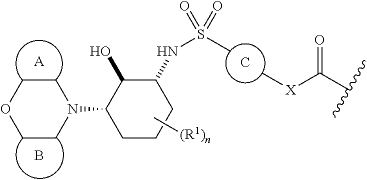

wherein: each of A and B is independently an optionally substituted 6-membered carbocyclic aromatic ring or an optionally substituted 5-6 membered heteroaromatic ring; C is an optionally substituted phenylene or an optionally substituted 5-6 membered heteroarylene; X is a bond, —O—, —N(R²)—, or optionally substituted 2-5 membered heteroalkylene; R¹ represents independently for each occurrence halogen, C₁-C₆ alkyl, C₁-C₆ haloalkyl, C₃-C₆ cycloalkyl, hydroxyl, C₁-C₆ alkoxy, or cyano; R² is hydrogen or optionally substituted C₁-C₆ alkyl; and n is 0, 1, 2, or 3.

Embodiment 22 provides the compound of any of Embodiments 1-17, wherein the protein phosphatase ligand component of Formula (I) has the following formula:

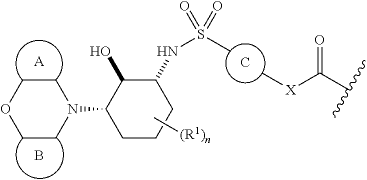

wherein: each of A and B is a 6-membered carbocyclic aromatic ring; C is phenylene; X is —N(R²)—; R¹ represents independently for each occurrence halogen, C₁-C₆ alkyl, or C₁-C₆ haloalkyl; R² is hydrogen or C₁-C₆ alkyl; and n is 0, 1, or 2.

Embodiment 23 provides the compound of any of Embodiments 1-22 wherein the protein phosphatase ligand component of Formula (I) is one of the following:

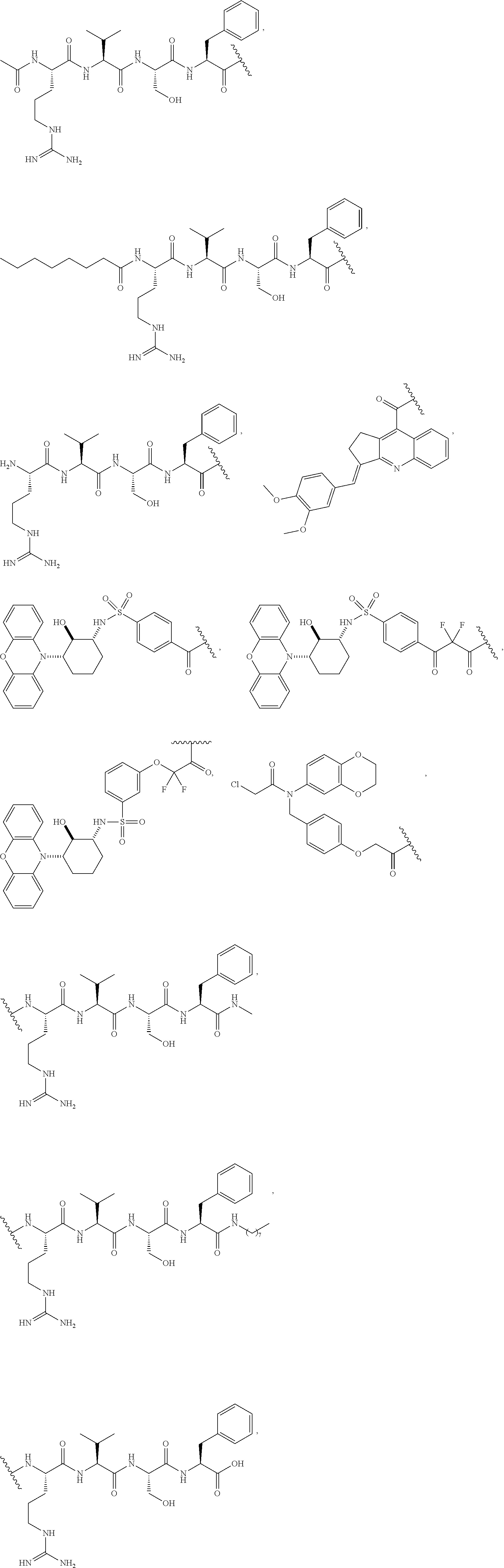

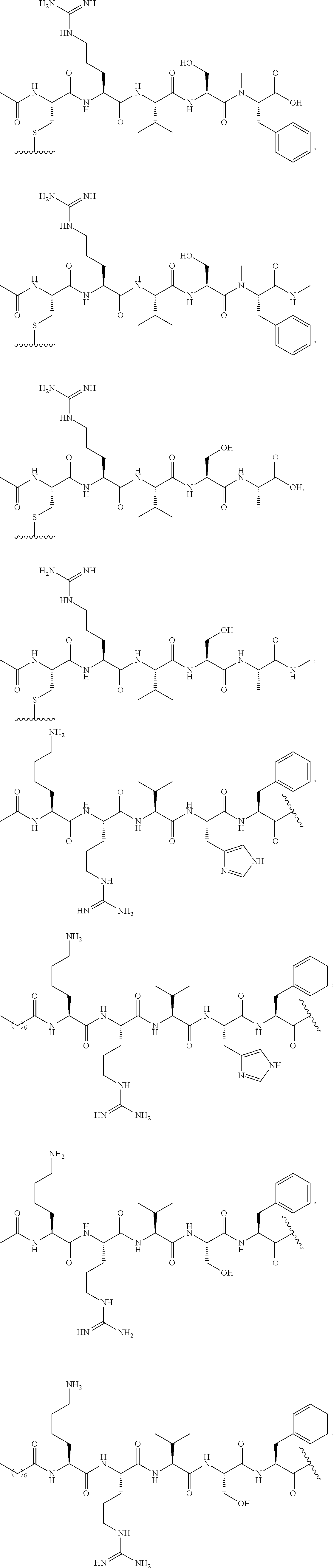

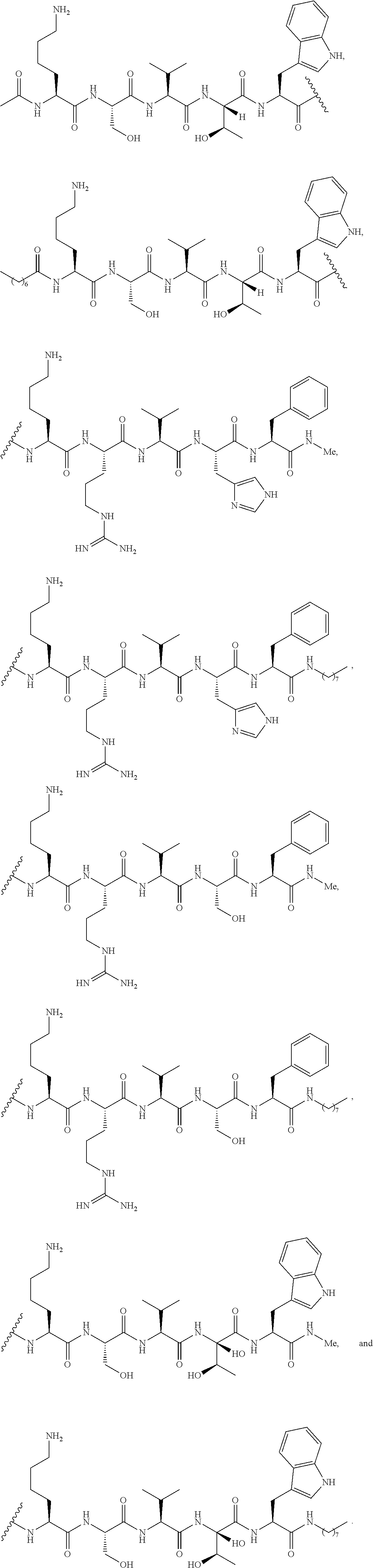

Embodiment 24 provides the compound of any of Embodiments 1-23, wherein the protein phosphatase ligand component of Formula (I) is one of the following:

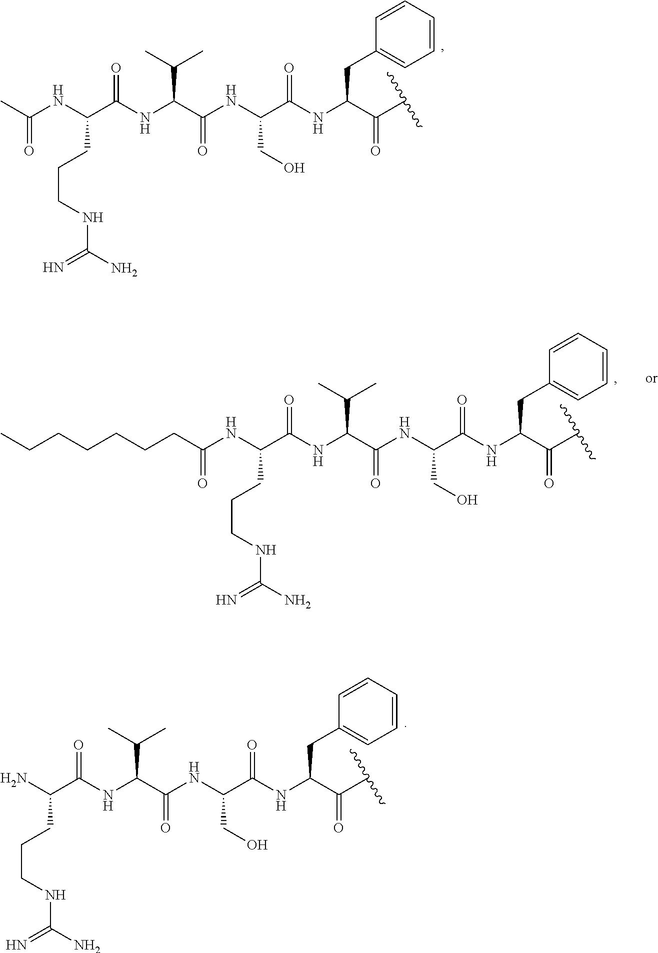 [SMILES: *C(=O)[C@H](Cc1ccccc1)NC(=O)[C@H](CO)NC(=O)[C@@H](NC(=O)[C@H](CCCNC(=N)N)NC(C)=O)C(C)C ; *C(=O)[C@H](Cc1ccccc1)NC(=O)[C@H](CO)NC(=O)[C@@H](NC(=O)[C@H](CCCNC(=N)N)NC(=O)CCCCCCC)C(C)C ; *C(=O)[C@H](Cc1ccccc1)NC(=O)[C@H](CO)NC(=O)[C@@H](NC(=O)[C@@H](N)CCCNC(=N)N)C(C)C]

Embodiment 25 provides the compound of any of Embodiments 1-24, wherein the target protein is involved in at least one biological role selected from the group consisting of cell proliferation, inflammation, and survival.

Embodiment 26 provides the compound of any of Embodiments 1-24, wherein the target protein is involved in the Tau aggregation pathway.

Embodiment 27 provides the compound of any of Embodiments 1-24, wherein the target protein is involved in the insulin signaling pathway.

Embodiment 28 provides the compound of any of Embodiments 1-27, wherein the target protein ligand binds to a protein listed in Table I-1.

Embodiment 29 provides the compound of any of Embodiments 1-28, wherein the target protein ligand binds to RAS, RAF, MEK, ERK, PI3K, AKT, A-RAF, B-RAF, C-RAF, ERK1, ERK2, RSK1, RSK2, PIM1, PKA, PKCI, PKCE, PRKD1, PKC, p38, BIM, NOXA, PUMA, BAD, BAK, BOK, TAU, CDK5, AMPK, GSK3beta, CK1, MARKs, Dyrk-1A, FYN, ABL, SYK, insulin receptor (IR), IRS1, mTOR, FoxO1, JNK, c-JUN, IKKβ, or NFkB.

Embodiment 30 provides the compound of any of Embodiments 1-29, wherein the target protein ligand binds to GSK-3beta, MDM2, MEK1, MEK2, TBK1, AKT1, AKT2, AKT3, RSK1, RSK3, RSK2, RSK4, SOS1, IRS1, Pyruvate kinase PKM, BAD, TAU, alpha-synuclein, STAT3, YAP, EGFR, BRAF, CRAF, PDK1, mTOR, KRAS, GYS1/2, HER2, Huntingtin, VHL, ITK, FGFR1, FGFR2, FGFR3, FGFR4, ERK-1, ERK-2, pyruvate kinase PKLR, or Brd4.

Embodiment 31 provides the compound of any of Embodiments 1-30, wherein the target protein ligand component of Formula (I) has the formula-(optionally substituted 3-10 membered heteroalkylene)-(optionally substituted C₁-C₁₀ alkylene)-Cl.

Embodiment 32 provides the compound of any of Embodiments 1-31, wherein the target protein ligand component of Formula (I) has the following formula:

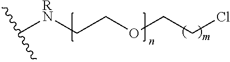

wherein: R is hydrogen or C₁-C₆ alkyl; m is 1-10; and n is 0, 1, 2, 3, or 4.

Embodiment 33 provides the compound of any of Embodiments 1-30, wherein the target protein ligand component of Formula (I) has the following formula:

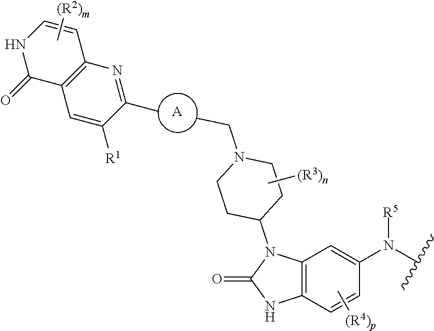

wherein: A is an optionally substituted phenylene or an optionally substituted 5-6 membered heteroarylene; R¹ is aryl, heteroaryl, or C₃-C₈ cycloalkyl, each of which is optionally substituted; R², R³, and R⁴ each represent independently for each occurrence halogen, C₁-C₆ alkyl, C₁-C₆ haloalkyl, C₃-C₆ cycloalkyl, hydroxyl, C₁-C₆ alkoxy, or cyano; R⁵ is hydrogen or optionally substituted C₁-C₆ alkyl; and m, n, and p are independently 0, 1, or 2.

Embodiment 34 provides the compound of any of Embodiments 1-30, wherein the target protein ligand component of Formula (I) is one of the following

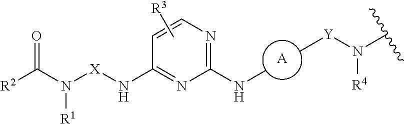

wherein: A is an optionally substituted phenylene or an optionally substituted 5-6 membered heteroarylene; R¹ and R⁴ are independently hydrogen or optionally substituted C₁-C₆ alkyl; R² is C₃-C₈ cycloalkyl, phenyl, or 5-6 membered heteroaryl, each of which is optionally substituted; R³ is halogen, C₁-C₆ alkyl, C₁-C₆ haloalkyl, C₃-C₆ cycloalkyl, hydroxyl, C₁-C₆ alkoxy, or cyano; X is optionally substituted C₂-C₆ alkylene; and Y is optionally substituted 3-6 membered heteroalkylene.

Embodiment 35 provides the compound of any of Embodiments 1-30, wherein the target protein ligand component of Formula (I) is one of the following

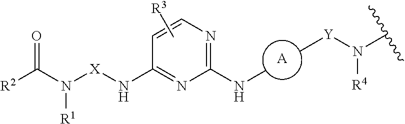

wherein: A is phenylene; R¹ and R⁴ are independently hydrogen or C₁-C₆ alkyl; R² is C₃-C₈ cycloalkyl; R³ is halogen, C₁-C₆ alkyl, C₁-C₆ haloalkyl, or cyano; X is C₂-C₆ alkylene; and Y is 3-6 membered heteroalkylene.

Embodiment 36 provides the compound of any of Embodiments 1-35, wherein the target protein ligand component of Formula (I) is one of the following:

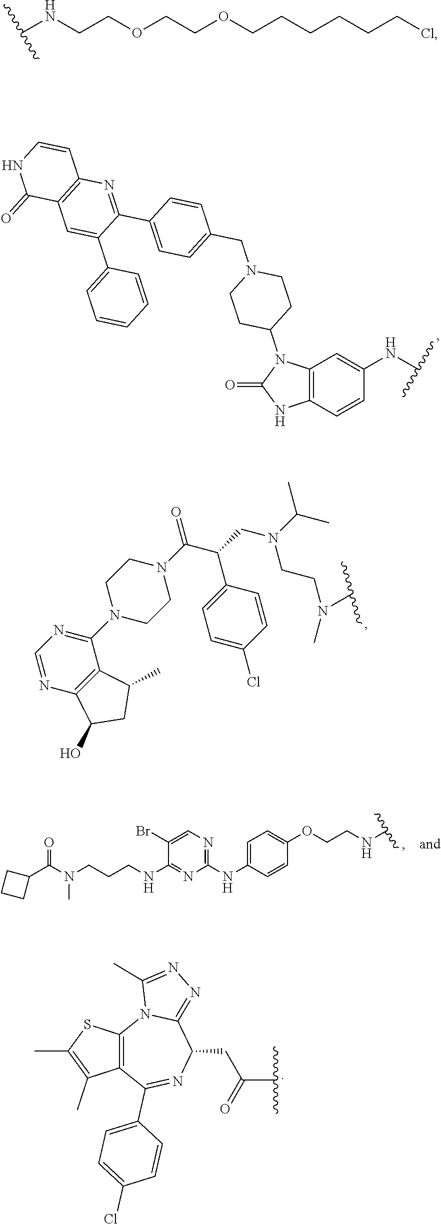 [SMILES: *NCCOCCOCCCCCCCl ; *Nc1ccc2[nH]c(=O)n(C3CCN(Cc4ccc(-c5nc6cc[nH]c(=O)c6cc5-c5ccccc5)cc4)CC3)c2c1 ; *N(C)CCN(C[C@@H](C(=O)N1CCN(c2ncnc3c2[C@H](C)C[C@H]3O)CC1)c1ccc(Cl)cc1)C(C)C ; *NCCOc1ccc(Nc2ncc(Br)c(NCCCN(C)C(=O)C3CCC3)n2)cc1 ; *C(=O)C[C@@H]1N=C(c2ccc(Cl)cc2)c2c(sc(C)c2C)-n2c(C)nnc21]

Embodiment 37 provides the compound of any of Embodiments 1-30, 33, and 36, wherein the target protein ligand component of Formula (I) is:

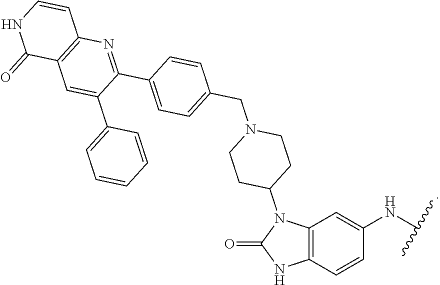 [SMILES: *Nc1ccc2[nH]c(=O)n(C3CCN(Cc4ccc(-c5nc6cc[nH]c(=O)c6cc5-c5ccccc5)cc4)CC3)c2c1]

Embodiment 38 provides the compound of any of Embodiments 1-30 and 34-36, wherein the target protein ligand component of Formula (I) is:

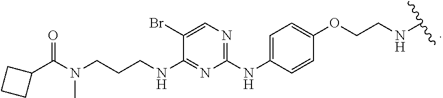 [SMILES: *NCCOc1ccc(Nc2ncc(Br)c(NCCCN(C)C(=O)C3CCC3)n2)cc1]

Embodiment 39 provides the compound of any of Embodiments 1-38, wherein the LINKER is a bond.

Embodiment 40 provides the compound of any of Embodiments 1-38, wherein the LINKER is a bivalent, saturated or unsaturated, straight or branched C₁₋₄₅ hydrocarbon chain, wherein 0-10 methylene units of the hydrocarbon are independently replaced with —O—, —S—, —N(R*)—, —OC(O)—, —C(O)O—, —S(O)—, —S(O)₂—, —N(R*)S(O)₂—, —S(O)₂N(R*)—, —N(R*)C(O)—, —C(O)N(R*)—, —OC(O)N(R*)—, —N(R*)C(O)O—, optionally substituted carbocyclyl, or optionally substituted heterocyclyl, wherein R* represents independently for each occurrence hydrogen, C₁₋₆ alkyl, or C₃₋₆ cycloalkyl.

Embodiment 41 provides the compound of any of Embodiments 1-38, wherein the LINKER has the formula —N(R)-(optionally substituted 3-20 membered heteroalkylene)ₚ-CH₂—C(O)— wherein R is hydrogen or optionally substituted C₁-C₆ alkyl, and p is 0 or 1.

Embodiment 42 provides the compound of any of Embodiments 1-38, wherein the LINKER has the formula —C(O)-(optionally substituted C₀-C₅ alkylene)-C(O)—N(R)-(optionally substituted 3-20 membered heteroalkylene)ₚ-CH₂—C(O)— wherein R is hydrogen or optionally substituted C₁-C₆ alkyl, and p is 0 or 1.

Embodiment 43 provides the compound of any of Embodiments 1-38, wherein the LINKER has the formula —CH₂-(optionally substituted C₀-C₅ alkylene)-C(O)—N(R)-(optionally substituted 3-20 membered heteroalkylene)ₚ-CH₂—C(O)— wherein R is hydrogen or optionally substituted C₁-C₆ alkyl, and p is 0 or 1.

Embodiment 44 provides the compound of any of Embodiments 1-43, wherein the LINKER is one of the following:

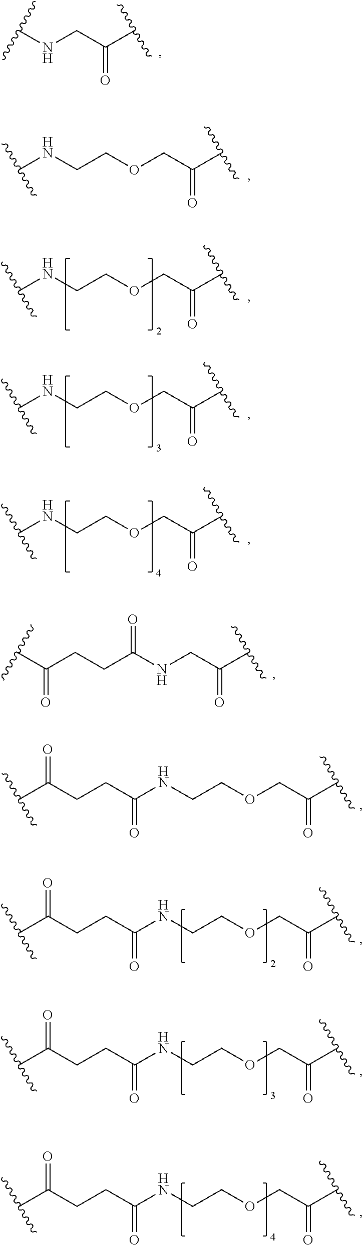

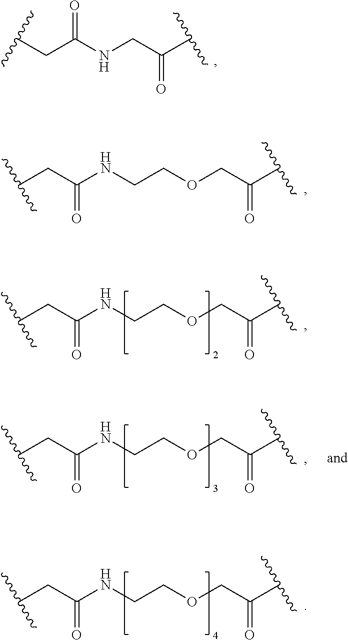

Embodiment 45 provides the compound of any of Embodiments 1-44, wherein the LINKER is:

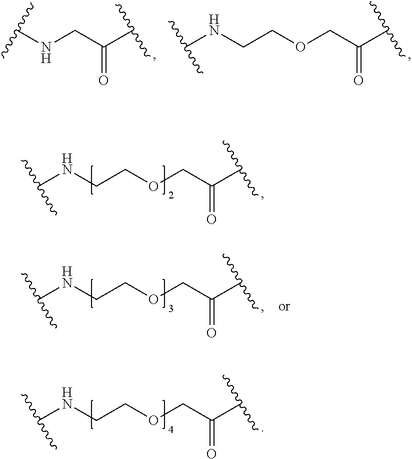

Embodiment 46 provides the compound of any of Embodiments 1-38, wherein the LINKER has the formula:

—(CH₂)ₘ₁—X₄—(CH₂—CH₂—X₅)ₘ₂—(CH₂)ₘ₃—C(X₆)— (VI),

wherein: (i) the target protein ligand is covalently bonded to —(CH₂)ₘ₁, and the protein phosphatase ligand is covalently bonded to C(X₆)—, or (ii) —(CH₂)ₘ₁ is covalently bonded to the protein phosphatase ligand, and C(X₆)— is covalently bonded to the target protein ligand; each m1, m2, and m3 is independently 0, 1, 2, 3, 4, 5, 6, 7, 8, 9, or 10; each X₄, X₅, and X₆ is independently absent (a bond), O, S, or N—R²⁰, wherein each R²⁰ is independently selected from the group consisting of hydrogen, optionally substituted C₁-C₆ alkyl, optionally substituted aryl, optionally substituted heteroaryl, optionally substituted C₃-C₈ cycloalkyl, and optionally substituted C₃-C₈ cycloheteroalkyl.

Embodiment 47 provides the compound of any of Embodiments 1-38 and 46, wherein the LINKER has the formula:

—(CH₂)ₘ₁—O—(CH₂—CH₂—O)ₘ₂—(CH₂)ₘ₃—C(O)— (VII).

Embodiment 48 provides the compound of any of Embodiments 1-38 and 46-47, wherein the LINKER has the formula:

—(CHR₂₁)ₘ₁—O—(CHR₂₂—CHR₂₃—O)ₘ₂—(CHR₂₄)ₘ₃—C(O)— (VIII),

wherein: (i) the target protein ligand is covalently bonded to —(CHR₂₁)ₘ₁, and the protein phosphatase ligand is covalently bonded to C(O)—, or (ii) —(CHR₂₁)ₘ₁ is covalently bonded to the protein phosphatase ligand, and C(O)— is covalently bonded to the target protein ligand; each m1, m2, and m3 is independently 0, 1, 2, 3, 4, 5, 6, 7, 8, 9, or 10; each R₂₁, R₂₂, R₂₃, and R₂₄ is independently selected from the group consisting of hydrogen, optionally substituted C₁-C₆ alkyl, optionally substituted aryl, optionally substituted heteroaryl, optionally substituted C₃-C₈ cycloalkyl, and optionally substituted C₃-C₈ cycloheteroalkyl.

Embodiment 49 provides the compound of any of Embodiments 1-38, wherein the LINKER comprises a polyethylene glycol chain ranging in size from about 1 to about 12 ethylene glycol units.

Embodiment 50 provides the compound of any of Embodiments 1-38, wherein the LINKER has the formula:

-(D-CON-D)ₘ₁- (IX),

wherein each D is independently a bond (absent) or —(CH₂)ₘ₁—Y—C(O)—Y—(CH₂)ₘ₁—; wherein m1 is 0, 1, 2, 3, 4, 5, 6, 7, 8, 9, or 10; wherein Y is O, S or N—R⁴; CON is a bond (absent), an optionally substituted C₃-C₈ cycloheteroalkyl, piperazinyl or a group selected from the group consisting of the following chemical structures:

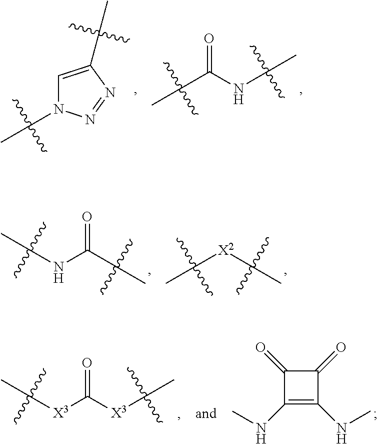

wherein X² is selected from the group consisting of O, S, NR⁴, S(O), S(O)₂, —S(O)₂O, —OS(O)₂, and OS(O)₂O; wherein X³ is selected from the group consisting of O, S, CHR⁴, and NR⁴; and wherein R⁴ is selected from the group consisting of H and a C₁-C₃ alkyl group optionally substituted with one or two hydroxyl groups.

Embodiment 51 provides the compound of any of Embodiments 1-50, wherein the LINKER is selected from the group consisting of: —NHCH₂CH₂(OCH₂CH₂)ₘOCH₂CH₂O—, wherein m is 0, 1, 2, 3, 4, 5, 6, 7, 8, 9, 10, 11, 12, 13, 14, 15, 16, 17, 18, 19, or 20; —NHCH₂CH₂(OCH₂CH₂)ₘOCH₂CH₂O(CH₂)ₙ—, wherein m and n are independently selected from the group consisting of 0, 1, 2, 3, 4, 5, 6, 7, 8, 9, 10, 11, 12, 13, 14, 15, 16, 17, 18, 19, and 20; and —(CH₂)ₙ₁(OCH₂CH₂)ₘ(CH₂)ₙ₂—, wherein m, n1, and n2 are independently selected from the group consisting of 0, 1, 2, 3, 4, 5, 6, 7, 8, 9, 10, 11, 12, 13, 14, 15, 16, 17, 18, 19, and 20.

Embodiment 52 provides the compound of any of Embodiments 1-51, wherein the (protein phosphatase ligand)-LINKER-group is selected from the group consisting of:

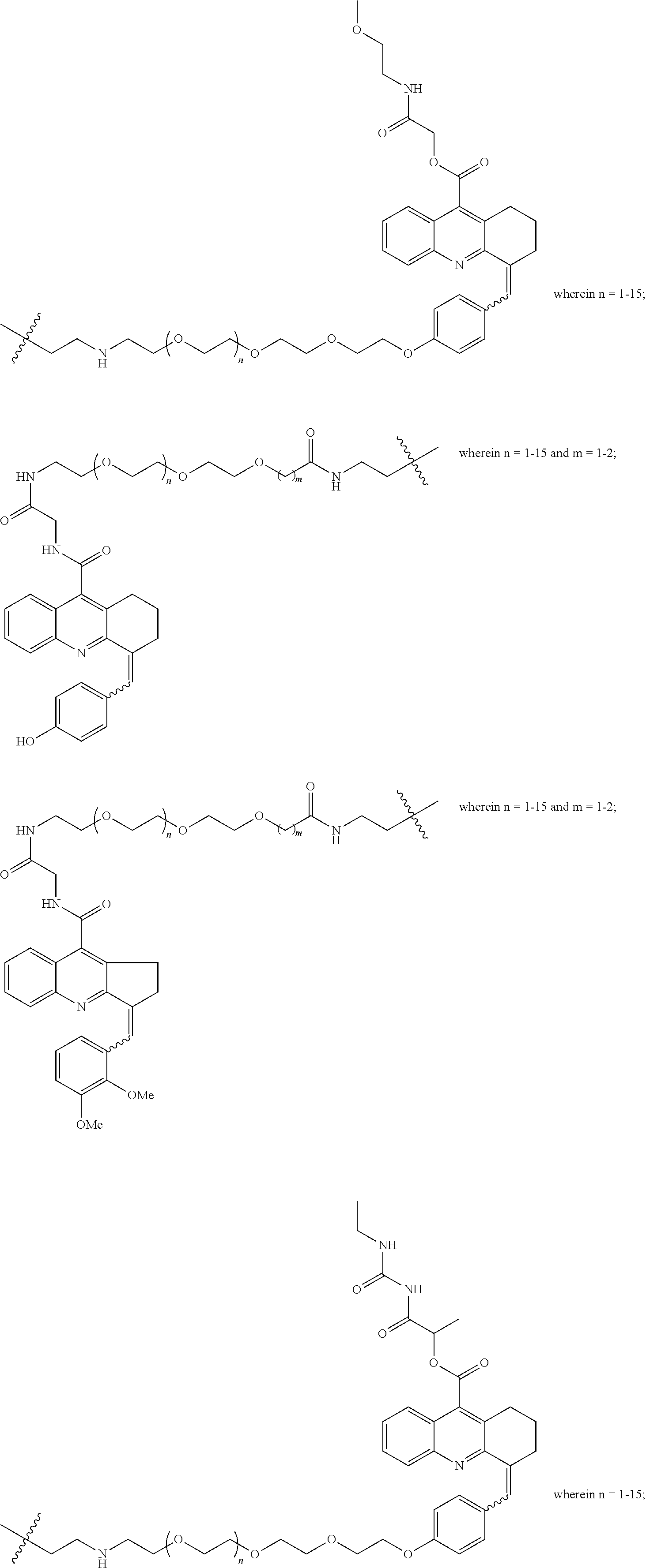

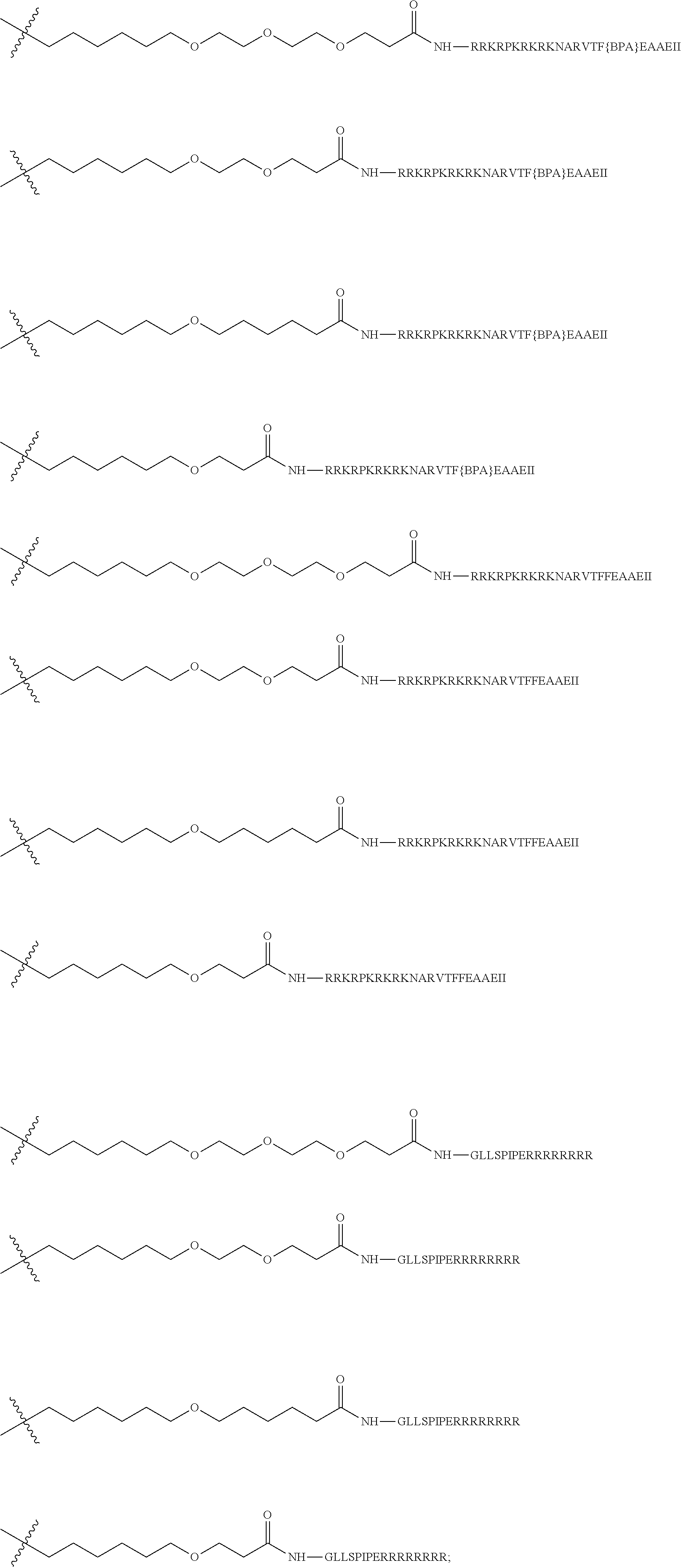

wherein BPA is L-4-benzoylphenyalanine.

Embodiment 53 provides a compound represented by one of the following formulae, or a pharmaceutically acceptable salt thereof:

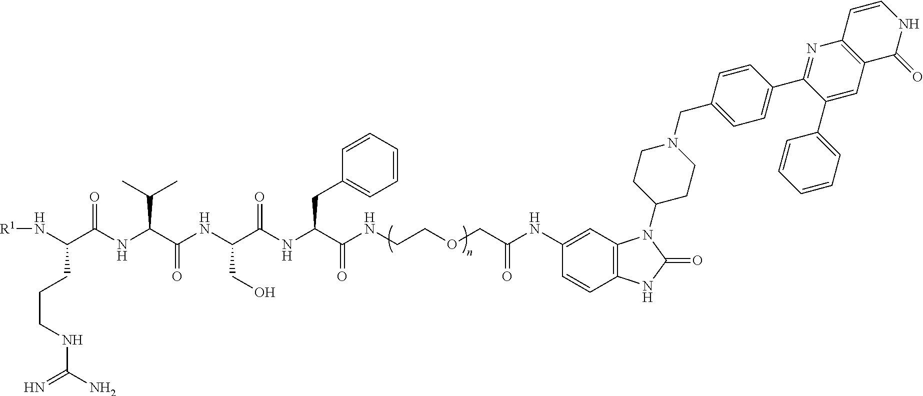

wherein R¹ is hydrogen or —C(O)CH₃, and n is 0, 1, 2, 3, or 4;

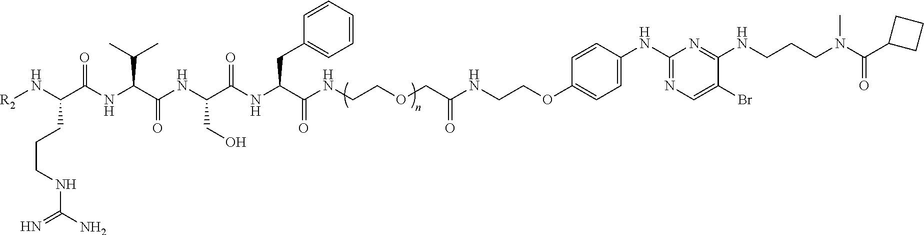

wherein R² is hydrogen, —C(O)CH₃, or —C(O)(CH₂)₆CH₃; and n is 0, 1, 2, 3, or 4; or

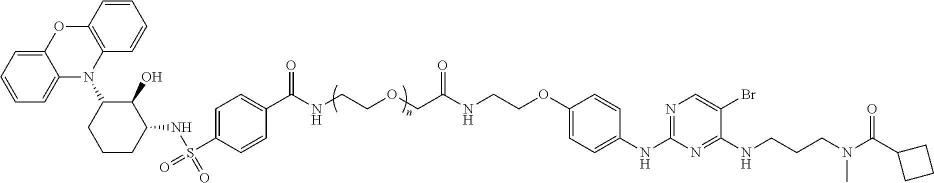

wherein n is 0, 1, 2, 3, or 4.

Embodiment 54 provides a compound in any one of Tables 1, 3, 4, 6, 7, 9, 11, 12, 14-16, or 18-20 herein, or a pharmaceutically acceptable salt thereof.

Embodiment 55 provides a compound in any one of Tables 24, 25, or 26 herein, or a pharmaceutically acceptable salt thereof.

Embodiment 56 provides a pharmaceutical composition comprising at least one compound of any one of Embodiments 1-55 and at least one pharmaceutically acceptable carrier.

Embodiment 57 provides the pharmaceutical composition of Embodiment 56, further comprising at least one additional therapeutic compound that treats or prevents a disease associated with and/or caused by overphosphorylation, undesirable phosphorylation, and/or uncontrolled phosphorylation of a target protein in a subject.

Embodiment 58 provides the pharmaceutical composition of Embodiment 57, wherein the disease comprises cancer, neurodegeneration, metabolic disease, diabetes, and/or insulin resistance.

Embodiment 59 provides a method of treating or preventing a disease associated with and/or caused by overphosphorylation, undesirable phosphorylation, and/or uncontrolled phosphorylation of a target protein in a subject, the method comprising administering to the subject a therapeutically effective amount of at least one compound of any one of Embodiments 1-55 and/or at least one pharmaceutical composition of any one of Embodiments 56-58.

Embodiment 60 provides the method of Embodiment 59, wherein the disease comprises cancer, neurodegeneration, metabolic disease, diabetes, and/or insulin resistance.

Embodiment 61 provides the method of any one of Embodiments 59-60, wherein the disease is cancer.

Embodiment 62 provides the method of any one of Embodiments 59-61, wherein the compound is administered to the subject by at least one route selected from the group consisting of nasal, inhalational, topical, oral, buccal, rectal, pleural, peritoneal, vaginal, intramuscular, subcutaneous, transdermal, epidural, intrathecal and intravenous routes.

Embodiment 63 provides the method of any one of Embodiments 59-62, wherein the subject is a human.

Embodiment 64 provides a method of dephosphorylating a target protein having a phosphate group, comprising exposing or contacting the target protein to a compound of any one of Embodiments 1-55 and/or at least one pharmaceutical composition of any one of Embodiments 56-58, to thereby dephosphorylate the target protein.

Embodiment 65 provides the method of claim 64, wherein the target protein is a target protein listed in Table I-1.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A compound of formula (I), or a salt, solvate, prodrug, isotopically labelled derivative, stereoisomer, tautomer, or geometric isomer thereof, and any mixtures thereof:
(protein phosphatase ligand)-LINKER-(target protein ligand) (I);
wherein the protein phosphatase ligand binds to a protein phosphatase, such that the protein phosphatase ligand does not significantly inhibit the phosphatase activity of the protein phosphatase;
wherein the target protein ligand binds to a target protein;
wherein the LINKER is selected such that it allows for the compound to bind simultaneously to the protein phosphatase and the target protein;
wherein, when the compound is simultaneously bound to the protein phosphatase and the target protein, the protein phosphatase is capable of dephosphorylating the target protein.

2. The compound of claim 1, wherein the protein phosphatase comprises at least one selected from the group consisting of protein phosphatase 1 (PP1), protein phosphatase 2A (PP2A), protein phosphatase 2B (PP2B), protein phosphatase 2C (PP2C), any of PTPRA through PTPRZ, and dual specific phosphatases DUSP1 through DUSP27.

3. (canceled)

4. The compound of claim 2, wherein the protein phosphatase is PP1 and wherein the protein phosphatase ligand comprises the amino acid sequence Arg Val Xaa Phe (SEQ ID NO: 1).

5. (canceled)

6. The compound of claim 2, wherein the protein phosphatase is PP2 and wherein the protein phosphatase ligand comprises the amino acid sequence Leu Ser Pro Ile Xaa Glu (SEQ ID NO:4).

7-9. (canceled)

10. The compound of claim 1, which comprises a compound of formula (IV), or a salt, solvate, prodrug, isotopically labelled derivative, stereoisomer, tautomer, or geometric isomer thereof, and any mixtures thereof: 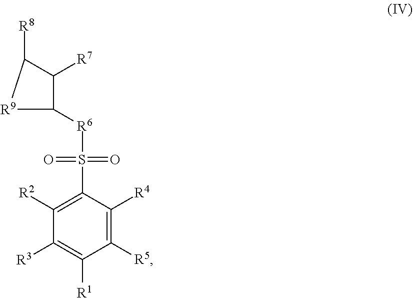
wherein:
R¹ is selected from the group consisting of H, C₁-C₆ haloalkyl, C₁-C₆ haloalkoxy, and -LINKER-(target protein ligand);
each one of R², R³, R⁴, and R⁵ is independently selected from the group consisting of H and -LINKER-(target protein ligand);
R⁶ is selected from the group consisting of —CH₂—, —CH(LINKER-target protein ligand)-, —NH—, and —N(LINKER-target protein ligand)-;
R⁷ is selected from the group consisting of H and OH;
R⁸ is selected from the group consisting of 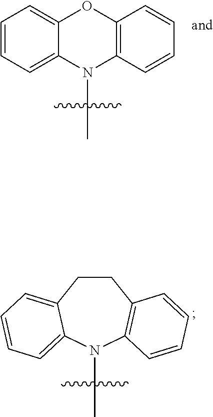 [SMILES: *N1c2ccccc2Oc2ccccc21 ; *N1c2ccccc2CCc2ccccc21]
R⁹ is selected from the group consisting of null (absent), —CH₂—, —CH₂CH₂—, and —CH₂CH₂CH₂—;
with the proviso that only one of R¹-R⁶ comprises -LINKER-(target protein ligand).

11-12. (canceled)

13. A compound of formula I-A, or a salt or solvate thereof:
(protein phosphatase ligand)-LINKER-(target protein ligand) (I-A);
wherein:
the protein phosphatase ligand binds to a protein phosphatase;
the target protein ligand binds to a target protein;
the LINKER is a bond or a group that allows the compound to bind to the protein phosphatase and the target protein.

14. The compound of claim 13, wherein the protein phosphatase ligand does not significantly inhibit phosphatase activity of the protein phosphatase.

15. The compound of claim 13, wherein the protein phosphatase ligand binds to protein phosphatase 1 (PP1).

16. The compound of claim 13, wherein the protein phosphatase ligand binds protein phosphatase 2A (PP2A), protein phosphatase 2B (PP2B), or protein phosphatase 2C (PP2C).

17. The compound of claim 13, wherein the protein phosphatase comprises at least one selected from the group consisting of CDC25A, CDC25B, CDC25C, ACP1, and Eya1 through Eya4.

18. The compound of claim 13, wherein the protein phosphatase ligand component of Formula I has the following formula: 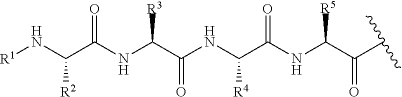
wherein:
R¹ is hydrogen or an optionally substituted group selected from —C(═O)(C₁-C₈ alkyl), —C(═O)(C₃-C₈ cycloalkyl), —C(═O)(C₀-C₃ alkylene)-aryl, and C₁-C₈ alkyl;
R² is optionally substituted —(C₁-C₈ alkylene)-N(H)—C(═NH)NH₂;
R³ is optionally substituted C₁-C₈ alkyl;
R⁴ is optionally substituted C₁-C₈ hydroxyalkyl; and
R⁵ is optionally substituted —(C₀-C₃ alkylene)-aryl or optionally substituted —(C₀-C₃ alkylene)-heteroaryl.

19. The compound of claim 13, wherein the protein phosphatase ligand component of Formula I has the following formula: 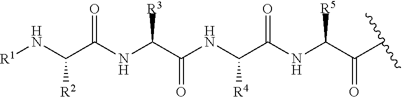
wherein:
R¹ is hydrogen or —C(═O)(C₁-C₈ alkyl);
R² is —(C₁-C₈ alkylene)-N(H)—C(═NH)NH₂;
R³ is C_{1-C} alkyl;
R⁴ is C₁-C₈ hydroxyalkyl; and
R⁵ is —(C₀-C₃ alkylene)-aryl.

20. (canceled)

21. The compound of claim 13, wherein the protein phosphatase ligand component of Formula I has the following formula: 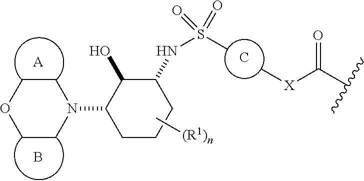
wherein:
each of A and B is independently an optionally substituted 6-membered carbocyclic aromatic ring or an optionally substituted 5-6 membered heteroaromatic ring;
C is an optionally substituted phenylene or an optionally substituted 5-6 membered heteroarylene;
X is a bond, —O—, —N(R²)—, or optionally substituted 2-5 membered heteroalkylene;
R¹ is independently for each occurrence halogen, C₁-C₆ alkyl, C₁-C₆ haloalkyl, C₃-C₆ cycloalkyl, hydroxyl, C₁-C₆ alkoxy, or cyano;
R² is hydrogen or optionally substituted C₁-C₆ alkyl; and
n is 0, 1, 2, or 3.

22. The compound of claim 13, wherein the protein phosphatase ligand component of Formula I has the following formula: 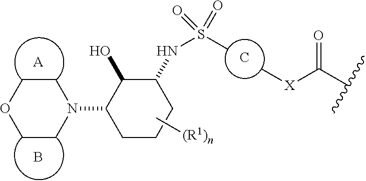
wherein:
each of A and B is a 6-membered carbocyclic aromatic ring;
C is phenylene;
X is —N(R²)—;
R¹ is independently for each occurrence halogen, C₁-C₆ alkyl, or C₁-C₆ haloalkyl;
R² is hydrogen or C₁-C₆ alkyl; and
n is 0, 1, or 2.

23. The compound of claim 13, wherein the protein phosphatase ligand component of Formula I is one of the following:

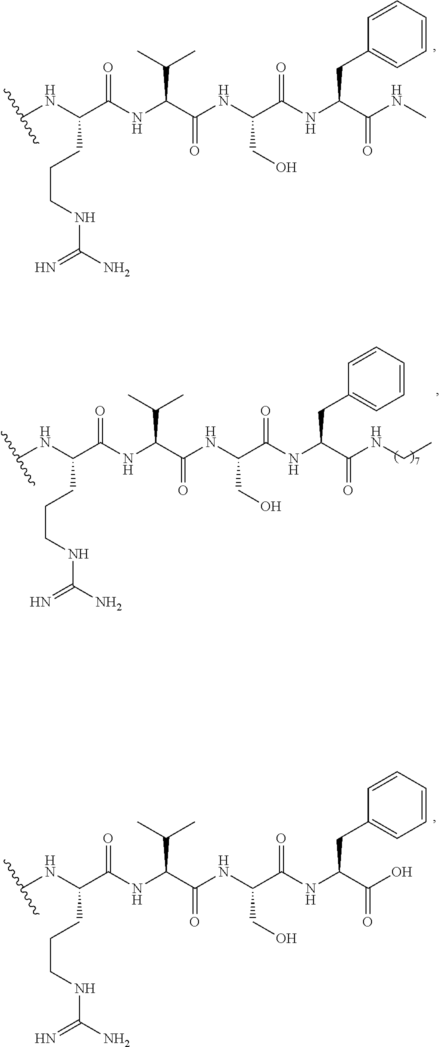

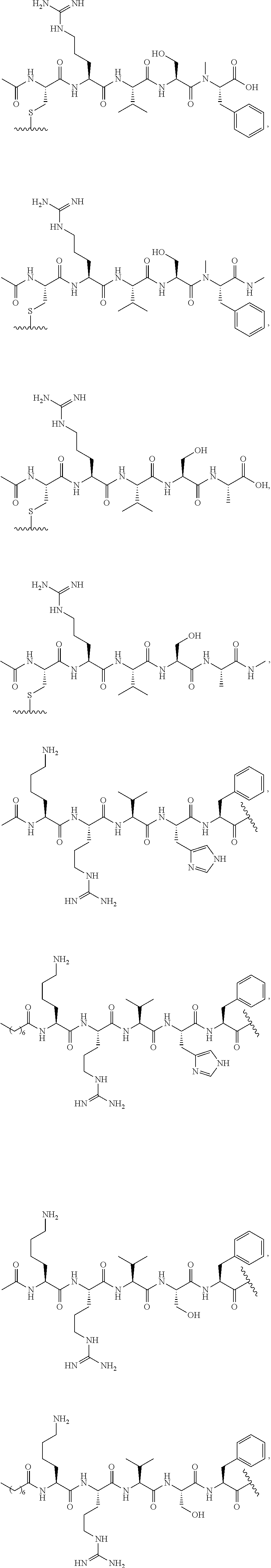

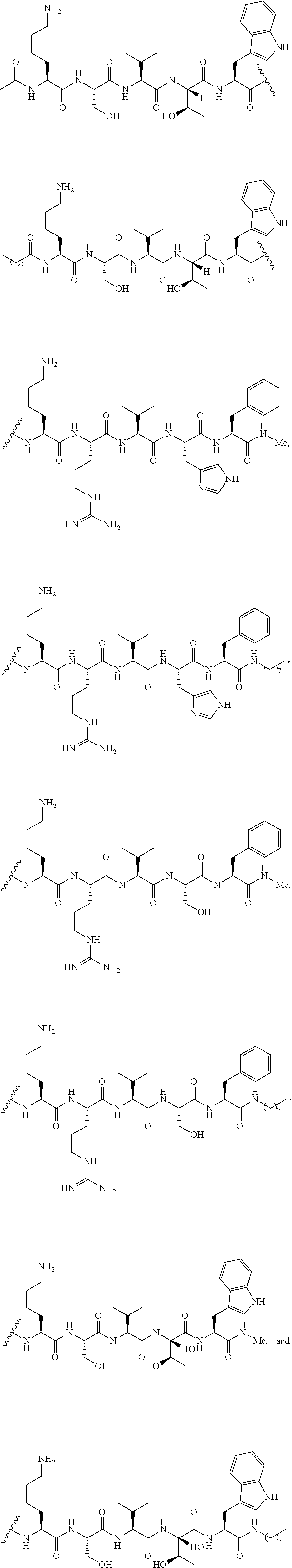

24. The compound of claim 13, wherein the protein phosphatase ligand component of Formula I is one of the following:

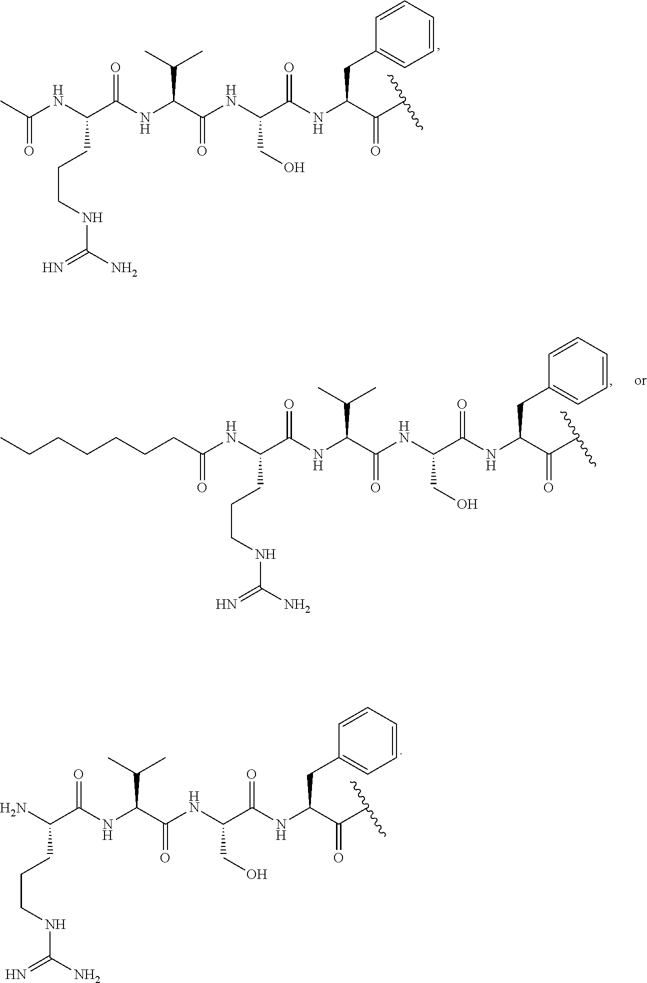 [SMILES: *C(=O)[C@H](Cc1ccccc1)NC(=O)[C@H](CO)NC(=O)[C@@H](NC(=O)[C@H](CCCNC(=N)N)NC(C)=O)C(C)C ; *C(=O)[C@H](Cc1ccccc1)NC(=O)[C@H](CO)NC(=O)[C@@H](NC(=O)[C@H](CCCNC(=N)N)NC(=O)CCCCCCC)C(C)C ; *C(=O)[C@H](Cc1ccccc1)NC(=O)[C@H](CO)NC(=O)[C@@H](NC(=O)[C@@H](N)CCCNC(=N)N)C(C)C]

25. The compound of claim 13, wherein the target protein is involved in at least one biological role selected from the group consisting of cell proliferation, inflammation, and survival.

26. The compound of claim 13, wherein the target protein is involved in the Tau aggregation pathway.

27. (canceled)

28. The compound of claim 13, wherein the target protein ligand binds to a protein listed in Table I-1.

29. The compound of claim 13, wherein the target protein ligand binds to RAS, RAF, MEK, ERK, PI3K, AKT, A-RAF, B-RAF, C-RAF, ERK1, ERK2, RSK1, RSK2, PIM1, PKA, PKCI, PKCE, PRKD1, PKC, p38, BIM, NOXA, PUMA, BAD, BAK, BOK, TAU, CDK5, AMPK, GSK3beta, CK1, MARKs, Dyrk-1A, FYN, ABL, SYK, insulin receptor (IR), IRS1, mTOR, FoxO1, JNK, c-JUN, IKKβ, or NFkB.

30. The compound of claim 13, wherein the target protein ligand binds to GSK-3beta, MDM2, MEK1, MEK2, TBK1, AKT1, AKT2, AKT3, RSK1, RSK3, RSK2, RSK4, SOS1, IRS1, Pyruvate kinase PKM, BAD, TAU, alpha-synuclein, STAT3, YAP, EGFR, BRAF, CRAF, PDK1, mTOR, KRAS, GYS1/2, HER2, Huntingtin, VHL, ITK, FGFR1, FGFR2, FGFR3, FGFR4, ERK-1, ERK-2, pyruvate kinase PKLR, or Brd4.

31-32. (canceled)

33. The compound of claim 13, wherein the target protein ligand component of Formula I has the following formula: 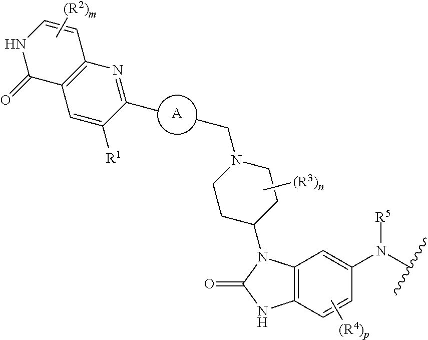
wherein:
A is an optionally substituted phenylene or an optionally substituted 5-6 membered heteroarylene;
R¹ is aryl, heteroaryl, or C₃-C₅ cycloalkyl, each of which is optionally substituted;
each of R², R³, and R⁴ is independently for each occurrence halogen, C₁-C₆ alkyl, C₁-C₆ haloalkyl, C₃-C₆ cycloalkyl, hydroxyl, C₁-C₆ alkoxy, or cyano;
R⁵ is hydrogen or optionally substituted C₁-C₆ alkyl; and
each of m, n, and p is independently 0, 1, or 2.

34. The compound of claim 13, wherein the target protein ligand component of Formula I is one of the following 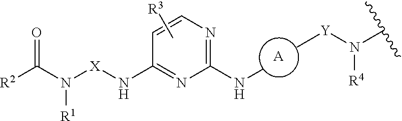
wherein:
A is an optionally substituted phenylene or an optionally substituted 5-6 membered heteroarylene;
R¹ and R⁴ are independently hydrogen or optionally substituted C₁-C₆ alkyl;
R² is C₃-C₈ cycloalkyl, phenyl, or 5-6 membered heteroaryl, each of which is optionally substituted;
R³ is halogen, C₁-C₆ alkyl, C₁-C₆ haloalkyl, C₃-C₆ cycloalkyl, hydroxyl, C₁-C₆ alkoxy, or cyano;
X is optionally substituted C₂-C₆ alkylene; and
Y is optionally substituted 3-6 membered heteroalkylene.

35. The compound of claim 13, wherein the target protein ligand component of Formula I is one of the following 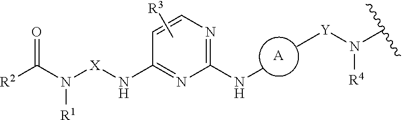
wherein:
A is phenylene;
R¹ and R⁴ are independently hydrogen or C₁-C₆ alkyl;
R² is C₃-C₈ cycloalkyl;
R³ is halogen, C₁-C₆ alkyl, C₁-C₆ haloalkyl, or cyano;
X is C₂-C₆ alkylene; and
Y is 3-6 membered heteroalkylene.

36. (canceled)

37. The compound of claim 13, wherein the target protein ligand component of Formula I is:

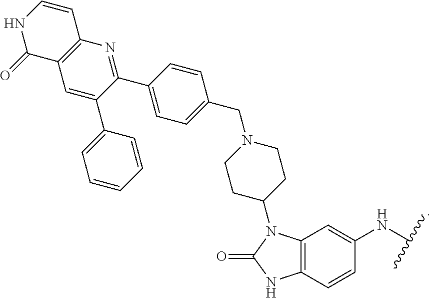 [SMILES: *Nc1ccc2[nH]c(=O)n(C3CCN(Cc4ccc(-c5nc6cc[nH]c(=O)c6cc5-c5ccccc5)cc4)CC3)c2c1]

38. The compound of claim 13, wherein the target protein ligand component of Formula I is:

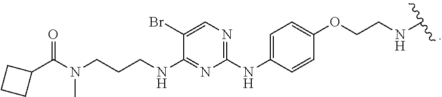 [SMILES: *NCCOc1ccc(Nc2ncc(Br)c(NCCCN(C)C(=O)C3CCC3)n2)cc1]

39. (canceled)

40. The compound of claim 13, wherein the LINKER is a bivalent, saturated or unsaturated, straight or branched C₁₋₄₅ hydrocarbon chain, wherein 0-10 methylene units of the hydrocarbon are independently replaced with —O—, —S—, —N(R*)—, —OC(O)—, —C(O)O—, —S(O)—, —S(O)₂—, —N(R*)S(O)₂—, —S(O)₂N(R*)—, —N(R*)C(O)—, —C(O)N(R*)—, —OC(O)N(R*)—, —N(R*)C(O)O—, optionally substituted carbocyclyl, or optionally substituted heterocyclyl, wherein R* is independently for each occurrence hydrogen, C₁₆ alkyl, or C₃₋₆ cycloalkyl.

41. The compound of claim 13, wherein the LINKER has the formula —N(R)-(optionally substituted 3-20 membered heteroalkylene)ₚ-CH₂—C(O)— wherein R is hydrogen or optionally substituted C₁-C₆ alkyl, and p is 0 or 1.

42-43. (canceled)

44. The compound of claim 13, wherein the LINKER is one of the following:

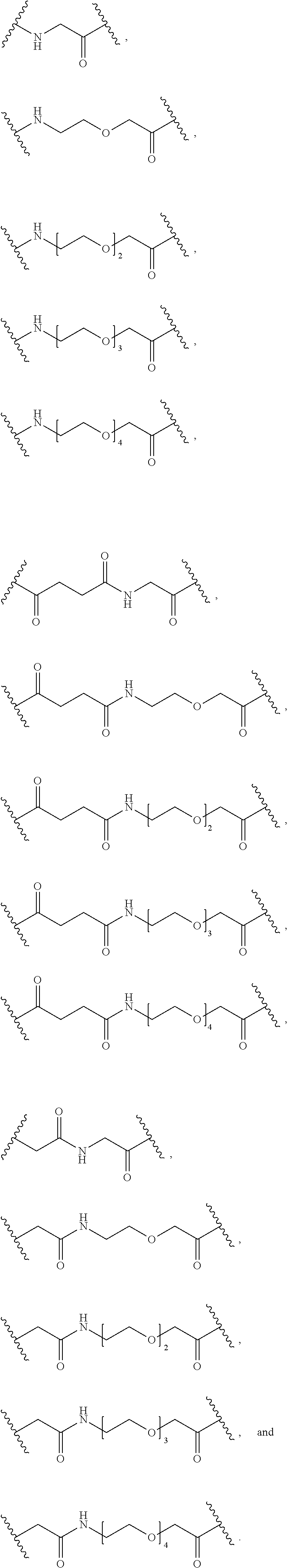

45. The compound of claim 13, wherein the LINKER is

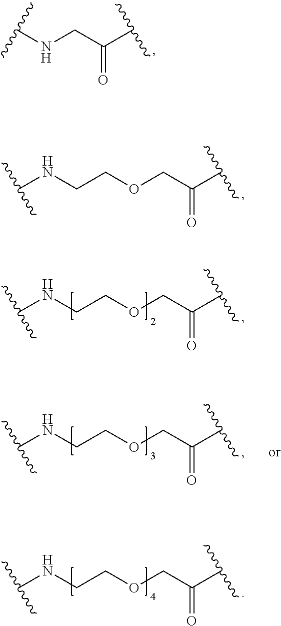

46. The compound of claim 1, wherein the LINKER has the formula:
—(CH₂)ₘ₁—X₄—(CH₂—CH₂—X₅)ₘ₂—(CH₂)ₘ₃—C(X₆)— (VI),
wherein:
(i) the target protein ligand is covalently bonded to —(CH₂)ₘ₁, and the protein phosphatase ligand is covalently bonded to C(X₆)—, or
(ii) —(CH₂)ₘ₁ is covalently bonded to the protein phosphatase ligand, and C(X₆)— is covalently bonded to the target protein ligand;
each m1, m2, and m3 is independently 0, 1, 2, 3, 4, 5, 6, 7, 8, 9, or 10;
each X₄, X₅, and X₆ is independently absent (a bond), O, S, or N—R²⁰,
wherein each R²⁰ is independently selected from the group consisting of hydrogen,
optionally substituted C₁-C₆ alkyl, optionally substituted aryl, optionally substituted heteroaryl, optionally substituted C₃-C₈ cycloalkyl, and optionally substituted C₃-C₈ cycloheteroalkyl.

47-51. (canceled)

52. The compound of claim 13, wherein the (protein phosphatase ligand)-LINKER-group is selected from the group consisting of: 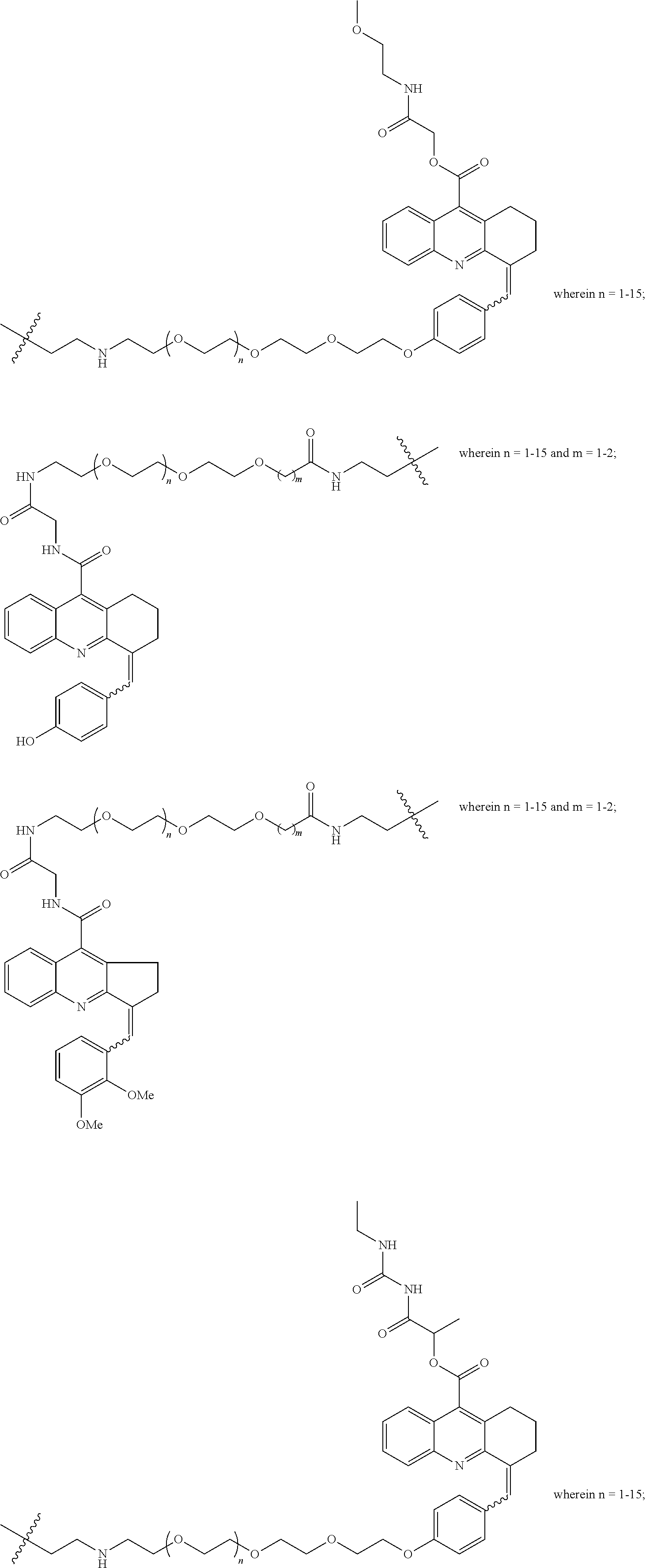 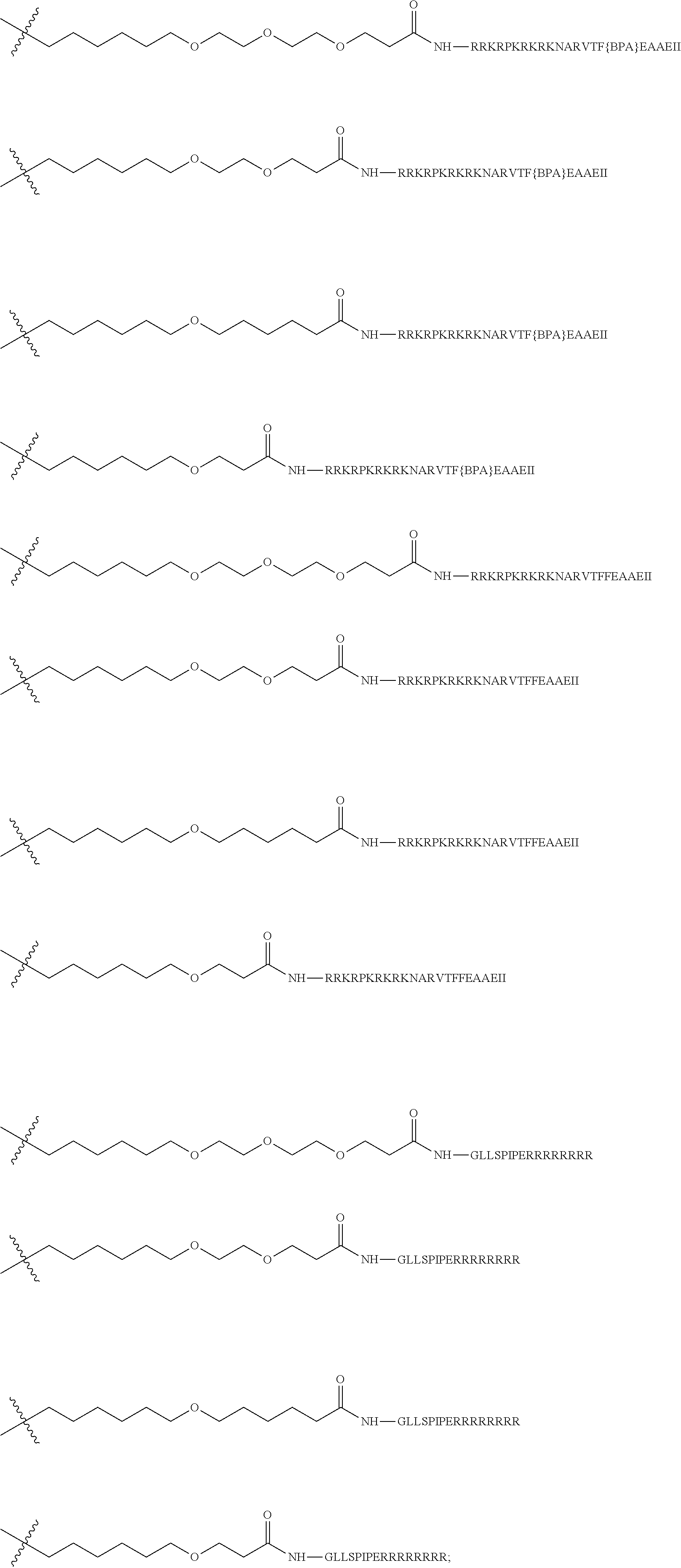
wherein BPA is L-4-benzoylphenyalanine.

53. A compound represented by one of the following formulae, or a pharmaceutically acceptable salt thereof: 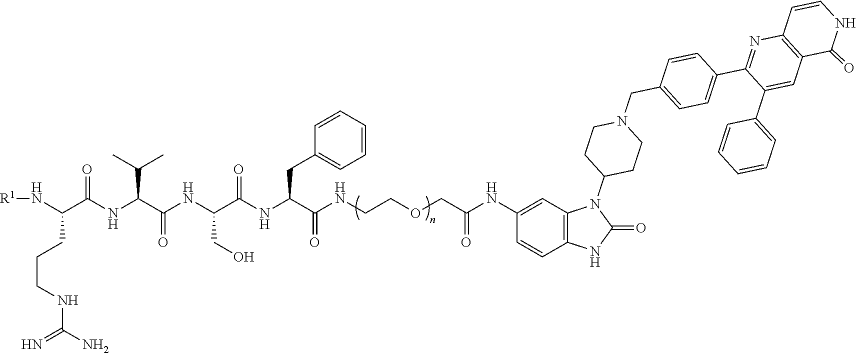
wherein R¹ is hydrogen or —C(O)CH₃; and n is 0, 1, 2, 3, or 4; 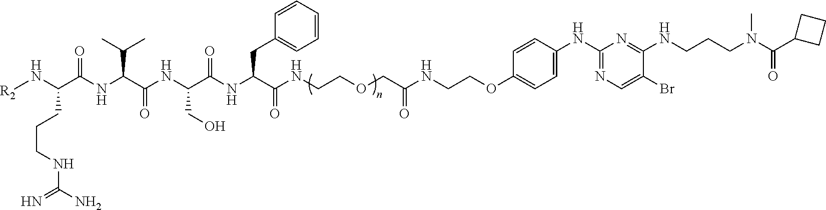
wherein R² is hydrogen, —C(O)CH₃, or —C(O)(CH₂)₆CH₃; and n is 0, 1, 2, 3, or 4; or 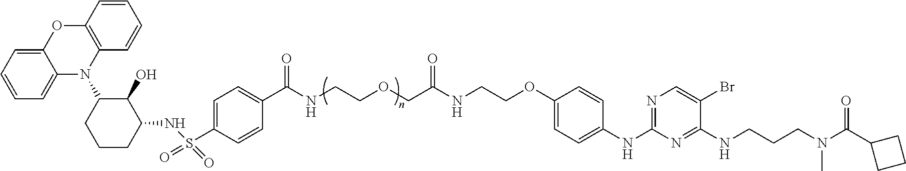
wherein n is 0, 1, 2, 3, or 4.

54. The compound of claim 13, wherein the compound is a compound in any one of Tables 1, 3, 4, 6, 7, 9, 11, 12, 14-16, or 18-20 herein, or a pharmaceutically acceptable salt thereof.

55. The compound of claim 13, wherein the compound is a compound in any one of Tables 24, 25, or 26 herein, or a pharmaceutically acceptable salt thereof.

56. A pharmaceutical composition comprising at least one compound of claim 1 and at least one pharmaceutically acceptable carrier.

57-58. (canceled)

59. A method of treating or preventing a disease associated with or caused by overphosphorylation, undesirable phosphorylation, or uncontrolled phosphorylation of a target protein in a subject, the method comprising administering to the subject a therapeutically effective amount of at least one compound of claim 1.

60. (canceled)

61. The method of claim 59, wherein the disease is cancer.

62. (canceled)

63. The method of claim 61, wherein the subject is a human.

64. A method of dephosphorylating a target protein having a phosphate group, comprising exposing or contacting the target protein to a compound of claim 1, to thereby dephosphorylate the target protein.

65. (canceled)


SEQUENCE LISTING (7 sequences)

SEQ ID NO: 1 - protein, 4 aa, Artificial Sequence
  Chemically synthesized
  MOD_RES (3)..(3): any natural or non-natural amino acid
RVXF

SEQ ID NO: 2 - protein, 23 aa, Artificial Sequence
  Chemically synthesized
  MOD_RES (17)..(17): L-4-benzoylphenylalanine
RRKRPKRKRKNARVTFXEAAEII

SEQ ID NO: 3 - protein, 23 aa, Artificial Sequence
  Chemically synthesized
RRKRPKRKRKNARVTFFEAAEII

SEQ ID NO: 4 - protein, 6 aa, Artificial Sequence
  Chemically synthesized
  MOD_RES (5)..(5): any natural or non-natural amino acid
LSPIXE

SEQ ID NO: 5 - protein, 16 aa, Artificial Sequence
  Chemically synthesized
GLLSPIPERRRRRRRR

SEQ ID NO: 6 - protein, 23 aa, Artificial Sequence
  Chemically synthesized
  MOD_RES (17)..(17): L-4-benzoylphenylalanine
RRKRPKRKRKNARATAXEAAEII

SEQ ID NO: 7 - protein, 16 aa, Artificial Sequence
  Chemically synthesized
GLASPAPERRRRRRRR